(19) United States
(12) Patent Application Publication
(10) Pub. No.: US 2008/0070268 A1
(43) Pub. Date: Mar. 20, 2008

(54) Differential expression profiling analysis of cell culture phenotypes and the uses thereof

(75) Inventors: Karin Anderson, Georgetown, MA (US); Niall Barron, Shankill (IE); Timothy Charlebois, Andover, MA (US); Martin Clynes, Clontarf (IE); Dana L. Di Nino, North Andover, MA (US); Padraig Doolan, Swords (IE); Patrick Gammell, Naas (IE); Kathleen Kopycinski, Somerville, MA (US); Mark Leonard, Manchester, NH (US); Kevin M. McCarthy, North Andover, MA (US); Paula Meleady, Ratoath (IE); Mark Melville, Melrose, MA (US); Martin S. Sinacore, Andover, MA (US)

Correspondence Address: Kirkpatrick & Lockhart Preston Gates Ellis LLP;(FORMERLY KIRKPATRICK & LOCKHART NICHOLSON GRAHAM), STATE STREET FINANCIAL CENTER, One Lincoln Street, BOSTON, MA, 02111-2950, US

(73) Assignees: Wyeth, Madison, NJ (US); Dublin City University, Dublin (IE)

(21) Appl. No.: 11/788,872

(22) Filed: Apr. 21, 2007

Related U.S. Application Data
(60) Provisional application No. 60/897,412, filed on Jan. 25, 2007.
(60) Provisional application No. 60/794,299, filed on Apr. 21, 2006.

(51) Int. Cl. C07H 21/04; C07K 16/00; C12N 5/04; C12Q 1/20

(52) U.S. Cl. 435/29; 435/325; 435/375; 530/350; 536/22.1

(57) ABSTRACT

The present invention provides methods for systematically identifying genes and proteins and related pathways that maximize protein expression and secretion by expression profiling analysis. The present invention further provides methods for manipulating the identified genes and proteins to engineer improved cell lines.

### CROSS-REFERENCE TO RELATED APPLICATIONS

This application claims priority to and the benefit of U.S. Provisional Patent Application No. 60/794,299, filed on Apr. 21, 2006, and U.S. Provisional Patent Application No. 60/897,412, filed on Jan. 25, 2007, the entire contents of both of which are incorporated by reference herein.

### REFERENCE TO SEQUENCE LISTING

This application includes as part of the originally filed subject matter two compact discs, labeled “Copy 1” and “Copy 2,” each disc containing a Sequence Listing. The machine format of each compact disc is IBM-PC and the operating system of each compact disc is MS-Windows. Each of the compact discs includes a single text file, which is named “WYE-060.ST25.txt” (1,423 KB, created Apr. 20, 2007). The contents of the compact discs labeled “Copy 1” and “Copy 2” are hereby incorporated by reference herein in their entireties.

### FIELD OF THE INVENTION

The present invention relates to methods for identifying genes and proteins that are involved in conferring a particular cell phenotype by differential expression profiling analysis and the use of the genes and proteins in the optimization of cell line culture conditions and transgene expression.

### BACKGROUND OF THE INVENTION

Fundamental to the present-day study of biology is the ability to optimally culture and maintain cell lines. Cell lines not only provide an in vitro model for the study of biological systems and diseases, but are also used to produce organic reagents. Of particular importance is the use of genetically engineered prokaryotic or eukaryotic cell lines to generate mass quantities of recombinant proteins. A recombinant protein may be used in a biological study, or as a therapeutic compound for treating a particular ailment or disease.

The production of recombinant proteins for biopharmaceutical application typically requires vast numbers of cells and/or particular cell culture conditions that influence cell growth and/or expression. In some cases, production of recombinant proteins benefits from the introduction of chemical inducing agents (such as sodium butyrate or valeric acid) to the cell culture medium. Identifying the genes and related genetic pathways that respond to the culture conditions (or particular agents) that increase transgene expression may elucidate potential targets that can be manipulated to increase recombinant protein production and/or influence cell growth.

Research into optimizing recombinant protein production has been primarily devoted to examining gene regulation, cellular responses, cellular metabolism, and pathways activated in response to unfolded proteins. Currently, there is no available method that allows for the simultaneous monitoring of transgene expression and identification of the genetic pathways involved in transgene expression. For example, currently available methods for detecting transgene expression include those that measure only the presence and amount of known proteins (e.g., Western blot analysis, enzyme-linked immunosorbent assay, and fluorescence-activated cell sorting), or the presence and amount of known messenger RNA (mRNA) transcripts (e.g., Northern blot analysis and reverse transcription-polymerase chain reaction). These and similar methods are not only limited in the number of known proteins and/or mRNA transcripts that can be detected at one time, but they also require that the investigator know or “guess” what genes are involved in transgene expression prior to experimentation (so that the appropriate antibodies or oligonucleotide probes are used). Another limitation inherent in blot analyses and similar protocols is that proteins or mRNA that are the same size cannot be distinguished. Considering the vast number of genes contained within a single genome, identification of even a minority of genes involved in a genetic pathway using the methods described above is costly and time-consuming. Additionally, the requirement that the investigator have some idea regarding which genes are involved does not allow for the identification of genes and related pathways that were either previously undiscovered or unknown to be involved in the regulation of transgene expression.

Therefore, there is a need in the field of cell line engineering for a more systematic approach to identify genes and proteins (including previously undiscovered genes and proteins) and related genetic pathways that are involved (directly or indirectly) with a particular cell culture phenotype, e.g., increased and efficient transgene expression. Discovery of these genes and/or related pathways will provide new targets that can be manipulated to improve the yield and quality of recombinant proteins and influence cell growth.

### SUMMARY OF THE INVENTION

The present invention solves these problems by providing differential expression profiling analysis of industrially relevant cell line phenotypes through the use of nucleic acid microarray and proteomics analysis methods. In particular, the present invention provides methods for systematically identifying genes and proteins and related pathways that maximize protein expression and secretion by expression profiling analysis. The present invention further provides methods for manipulating the identified genes and proteins to engineer improved cell lines.

Thus, in one aspect, the present invention features a method for identifying proteins regulating or indicative of a cell culture phenotype in a cell line. The method includes generating a protein expression profile of a sample derived from a test cell line; comparing the protein expression profile to a control profile derived from a control cell line; and identifying one or more differentially expressed proteins based on the comparison, wherein the test cell line has a cell culture phenotype distinct from that of the control cell line, and the one or more differentially expressed proteins are capable of regulating or indicating the cell culture phenotype. In a preferred embodiment, the cell line is a Chinese hamster ovary (CHO) cell line. In another embodiment, the protein expression profile is generated by fluorescent two-dimensional differential in-gel electrophoresis.

In some embodiments, the cell culture phenotype is a cell growth rate, a cellular productivity (such as a maximum cellular productivity or a sustained high cellular productivity), a peak cell density, a sustained cell viability, a rate of ammonia production or consumption, or a rate of lactate production or consumption. In one embodiment, the cell culture phenotype is a maximum cellular productivity. In another embodiment, the cell culture phenotype is a sustained cell viability. In yet another embodiment, the cell culture phenotype is a peak cell density. In still another embodiment, the cell culture phenotype is a cell growth rate.

The present invention provides a method for improving a cell line by modulating, i.e., up-regulating or down-regulating, one or more proteins identified according to the method described above. As used herein, “up-regulating” includes providing an exogenous nucleic acid (e.g., an over-expression construct) encoding a protein of interest or a variant retaining its activity (such as, for example, a mammalian homolog thereof, such as a primate or rodent homolog) or providing a factor or a molecule indirectly enhancing the protein or gene activity or expression level. As used herein, “down-regulating” includes knocking-out the gene encoding a protein of interest, providing an RNA interference construct, or providing an inhibitor or other factors indirectly inhibiting the protein or gene activity or expression level. In one particular embodiment, the present invention provides a method for improving a cell line by down-regulating one or more proteins identified according to the method described above by RNA interference.

In particular, the present invention provides a method for improving cellular productivity of a cell line including modulating, i.e., up-regulating or down-regulating, one or more proteins identified according to the method described above. In one embodiment, the present invention provides a method for improving cellular productivity of a cell line including modulating, i.e., up-regulating or down-regulating, one or more genes or proteins selected from Tables 2, 3, 9, 10, 11, and 12.

In one embodiment, the present invention provides a method for improving the cell growth rate of a cell line including modulating, i.e., up-regulating or down-regulating, one or more proteins identified according to the method described above. In particular, the present invention provides a method for improving the cell growth rate of a cell line including modulating, i.e., up-regulating or down-regulating, one or more genes or proteins selected from Tables 4, 5, 6, 13, 14, 27 and 28.

In another embodiment, the present invention provides a method for increasing the peak cell density of a cell line including modulating, i.e., up-regulating or down-regulating, one or more proteins identified according to the method described above. In particular, the present invention provides a method for increasing the peak cell density of a cell line including modulating, i.e., up-regulating or down-regulating, one or more genes or proteins selected from Tables 8, 15, 16, and 17.

In another embodiment, the present invention provides a method for increasing the sustained cell viability of a cell line including modulating, i.e., up-regulating or down-regulating, one or more proteins identified according to the method described above. In particular, the present invention provides a method for increasing the sustained cell viability of a cell line including modulating, i.e., up-regulating or down-regulating, one or more genes or proteins selected from Tables 7, 18 and 19.

In another embodiment, the present invention provides a method for regulating the lactate production or consumption of a cell line including modulating, i.e., up-regulating or down-regulating, one or more proteins identified according to the method described above. In particular, the present invention provides a method for regulating the lactate production or consumption of a cell line including modulating, i.e., up-regulating or down-regulating, one or more genes or proteins selected from Tables 7, 18 and 19.

In yet another embodiment, the present invention provides a method for improving a cell line by modulating, i.e., up-regulating or down-regulating, one or more genes or proteins identified according to the method described above. In particular, the present invention provides a method for improving a cell line by modulating, i.e., up-regulating or down-regulating, one or more genes or proteins selected from Tables 20, 24, 25 and 26.

In another aspect, the present invention provides a method for improving a cell line by modulating, i.e., up-regulating or down-regulating, at least two genes or proteins, wherein a first gene or protein affects a first cell culture phenotype and a second gene or protein affects a second, different cell culture phenotype, wherein the cell culture phenotypes are selected from the group consisting of a cell growth rate, a cellular productivity, a peak cell density, a sustained cell viability, a rate of ammonia production or consumption, or a rate of lactate production or consumption. In one embodiment, the method further including up-regulating or down-regulating a third gene or protein affecting a third cell culture phenotype different from the first and second cell culture phenotypes.

In yet another aspect, the present invention provides a method of assessing a cell culture phenotype of a cell line. The method including detecting, in a sample from the cell culture, an expression level of a protein identified according to any of the methods described above; and comparing the expression level to a reference level, wherein the comparison is indicative of the cell culture phenotype.

Alternatively, the present invention provides a method of assessing a cell culture phenotype of a cell line. The method including detecting, in a sample from the cell culture, one or more markers indicative of the cell culture phenotype, wherein the markers are selected from the group consisting of peptides selected from FIGS. 7 through 138, or genes or proteins selected from Tables 1 through 20 and Tables 24 through 30.

In another aspect, the present invention provides an engineered cell line with an improved cell culture phenotype containing a population of engineered cells, each of which comprises an engineered construct up-regulating or down-regulating one or more proteins identified according to various methods as described above. In particular, the present invention provides an engineered cell line with an improved cellular productivity containing a population of engineered cells, each of which comprises an engineered construct up-regulating or down-regulating one or more genes or proteins selected from Tables 2, 3, and 9 through 12. In some embodiments, the engineered construct is an over-expression construct. In other embodiments, the engineered construct is an interfering RNA construct.

In other embodiments, the present invention provides an engineered cell line with an improved cell growth rate including a population of engineered cells, each of which includes an engineered construct up-regulating or down-regulating one or more genes or proteins selected from Tables 4, 5, 6, 13, 14, 27 and 28. In some embodiments, the engineered construct is an over-expression construct. In other embodiments, the engineered construct is an interfering RNA construct.

In other embodiments, the present invention provides an engineered cell line with an improved peak cell density containing a population of engineered cells, each of which includes an engineered construct up-regulating or down-regulating one or more genes or proteins selected from Tables 8, 15, 16, and 17. In some embodiments, the engineered construct is an over-expression construct. In other embodiments, the engineered construct is an interfering RNA construct.

In other embodiments, the present invention provides an engineered cell line with an improved sustained cell viability containing a population of engineered cells, each of which comprising an engineered construct up-regulating or down-regulating one or more genes or proteins selected from Tables 18 and 26. In some embodiments, the engineered construct is an over-expression construct. In other embodiments, the engineered construct is an interfering RNA construct.

In other embodiments, the present invention provides an engineered cell line with regulated lactate production or consumption containing a population of engineered cells, each of which comprising an engineered construct up-regulating or down-regulating one or more genes or proteins selected from Tables 29 and 30. In some embodiments, the engineered construct is an over-expression construct. In other embodiments, the engineered construct is an interfering RNA construct.

In some embodiments, the present invention provides an improved cell line containing a population of engineered cells, each of which comprising an engineered construct up-regulating or down-regulating one or more genes or proteins selected from Table 20, 24, 25 and 26. In some embodiments, the engineered construct is an over-expression construct. In other embodiments, the engineered construct is an interfering RNA construct.

In yet another aspect, the invention provides a method for expression of a protein of interest using engineered cell lines as described above. The method includes the steps of introducing into an engineered cell line according to any one of the embodiments described above a nucleic acid encoding the protein of interest; and harvesting the protein of interest.

In still another aspect, the invention also provides isolated genes or proteins, or polynucleotides or polypeptides that are of previously undiscovered genes or proteins, and/or are involved with regulating or indicative of cell culture phenotypes of interest. In particular, the invention provides an isolated or recombinant nucleic acid containing a sequence selected from Tables 9, 13, and 15, complements thereof, and subsequences thereof. The present invention also provides an isolated or recombinant protein containing a sequence selected from Tables 2 and 3, or fragments thereof. The invention also provides genetically engineered expression vectors, host cells, and transgenic animals comprising the nucleic acid molecules or proteins of the invention. The invention additionally provides inhibitory polynucleotides, e.g., antisense and RNA interference (RNAi) molecules, to the nucleic acid molecules of the invention or the nucleic acid encoding the proteins of the invention.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

FIG. 1 is a flowchart of an exemplary method for identifying genes and proteins of the invention.

FIG. 2 illustrates an exemplary matrix of CHO lines and cellular phenotypes.

FIG. 3 depicts an exemplary phenotypic comparison between test cell lines and control cell lines for a “high cell growth rate” phenotype.

FIG. 4 illustrates a method of protein expression profiling.

FIGS. 5 and 6 depict the Cy3 and Cy5 staining patterns on an exemplary gel and provide graphical depictions of the relative abundance of selected proteins. In FIG. 5, a protein that appears to be 5-fold upregulated in the Cy5-labeled test cell extract is outlined. In FIG. 6, a protein that appears to be 4-fold downregulated in the Cy5-labeled test cell extract is outlined.

FIGS. 7 through 138 illustrate sequence data and analysis for individual, differentially-expressed proteins.

FIGS. 139 and 140 schematically depict an unsupervised Pearson Clustering Analysis.

FIG. 141 depicts an exemplary method of data analysis using pairwise differences.

FIG. 142 depicts an exemplary method of data analysis that does not rely on pairwise differences.

FIGS. 143-146 depict exemplary evaluations of identified genes in the 3C7 cell line.

FIGS. 147 and 148 illustrate a 24 well format for assessing the impact of over-expression of identified genes on cellular growth and productivity.

Exemplary results of over-expression of identified genes on cellular growth and productivity are illustrated in FIGS. 149-151.

### DETAILED DESCRIPTION OF THE INVENTION

The present invention provides systematic methods for identifying genes and proteins that influence cell culture phenotypes of interest. The methods of the invention are based on differential expression profiling analysis of industrially relevant cell culture phenotypes through integrated use of DNA microarray and proteomics analysis. Specifically, the method includes generating a gene or protein expression profile of a sample derived from a test cell line; comparing the gene or protein expression profile to a control profile derived from a control cell line which has a cell culture phenotype distinct from that of the test cell line; and identifying one or more differentially expressed genes or proteins based on the comparison. As used herein, the test cell line and the control cell line can be different cell lines with different genetic background or same cell line grown under different cell culture conditions.

The one or more differentially expressed genes or proteins are candidate genes or proteins that regulate or are indicative of the cell culture phenotype of interest. The identified genes and proteins can be further confirmed and validated. The identified genes or proteins may also be manipulated to improve the cell culture phenotype of interest. Therefore, the present invention represents a significant advance in cell engineering for rational designing of improved cell lines and cell culture conditions.

Various aspects of the invention are described in further detail in the following subsections. The use of subsections is not meant to limit the invention. Each subsection may apply to any aspect of the invention. In this application, the use of “or” means “and/or” unless stated otherwise.

Cell Lines and Cell Culture Phenotypes

The present invention contemplates differential expression profiling analysis and optimization of cell lines derived from a variety of organisms, including, but not limited to, bacteria, plants, fungi, and animals (the latter including, but not limited to, insects and mammals). For example, the present invention may be applied to Escherichia coli, Spodoptera frugiperda, Nicotiana sp., Zea mays, Lemna sp., Saccharomyces sp., Pichia sp., Schizosaccharomyces sp., mammalian cells, including, but not limited to, COS cells, CHO cells, 293 cells, A431 cells, 3T3 cells, CV-1 cells, HeLa cells, L cells, BHK21 cells, HL-60 cells, U937 cells, HEK cells, PerC6 cells, Jurkat cells, normal diploid cells, cell strains derived from in vitro culture of primary tissue, and primary explants. The list of organisms and cell lines are meant only to provide nonlimiting examples.

In particular, the present invention contemplates differential expression profiling analysis of industrially relevant cell lines, such as, for example, CHO cells. CHO cells are a primary host for therapeutic protein production, such as, for example, monoclonal antibody production, receptor productions, and Fc fusion proteins because CHO cells provide fidelity of folding, processing, and glycosylation. CHO cells are also compatible with deep-tank, serum-free culture and have excellent safety records.

The present invention permits an understanding of pathways, genes and proteins that influence desired cell culture phenotypes or characteristics, for example, cell phenotypes that enable highly productive fed-batch processes. Such desired cell phenotypes include, but are not limited to, high cell growth rate, high peak cell density, sustained high cell viability, high maximum cellular productivity, sustained high cellular productivity, low ammonium production, and low lactate production. Desired phenotypes or characteristics may be inherent properties of established cell lines that have certain genomic backgrounds. Desired phenotypes or characteristics may also be conferred to cells by growing the cells in different conditions, e.g., temperatures, cell densities, the use of agents such as sodium butyrate, to be in different kinetic phases of growth (e.g., lag phase, exponential growth phase, stationary phase or death phase), and/or to become serum-independent, etc. During the period in which these phenotypes are induced, and/or after these phenotypes are achieved, a pool of target nucleic acid or protein samples can be prepared from the cells and analyzed with the oligonucleotide array to determine and identify which genes demonstrate altered expression in response to a particular stimulus (e.g., temperature, sodium butyrate), and therefore are potentially involved in conferring the desired phenotype or characteristic.

Preparation of Pool of Target Nucleic Acids

In order to conduct gene expression profiling analysis, a pool of target nucleic acids are prepared from a sample derived from a cell line. Any biological sample may be used as a source of target nucleic acids. The pool of target nucleic acids can be total RNA, or any nucleic acid derived therefrom, including each of the single strands of cDNA made by reverse transcription of the mRNA, or RNA transcribed from the double-stranded cDNA intermediate. Methods of isolating target nucleic acids for analysis with an oligonucleotide array or other probes, such as phenol-chloroform extraction, ethanol precipitation, magnetic bead separation, or silica-gel affinity purification, are well known to one of skill in the art.

For example, various methods are available for isolating or enriching RNA. These methods include, but are not limited to, RNeasy kits (provided by Qiagen), MasterPure kits (provided by Epicentre Technologies), charge-switch technology (see, e.g., U.S. Published patent application Nos. 2003/0054395 and 2003/0130499), and TRIZOL (provided by Gibco BRL). The RNA isolation protocols provided by Affymetrix can also be employed in the present invention. See, e.g., GeneChip® EXPRESSION ANALYSIS TECHNICAL MANUAL (701021 rev. 3, Affymetrix, Inc. 2002).

Preferably, the pool of target nucleic acids (i.e., mRNA or nucleic acids derived therefrom) should reflect the transcription of gene coding regions. In one example, mRNA is enriched by removing rRNA. Different methods are available for eliminating or reducing the amount of rRNA in a sample. For instance, rRNA can be removed by enzyme digestions. According to the latter method, rRNAs are first amplified using reverse transcriptase and specific primers to produce cDNA. The rRNA is allowed to anneal with the cDNA. The sample is then treated with RNAase H, which specifically digests RNA within an RNA:DNA hybrid.

Target nucleic acids may be amplified before incubation with an oligonucleotide array or other probes. Suitable amplification methods, including, but not limited to, reverse transcription-polymerase chain reaction, ligase chain reaction, self-sustained sequence replication, and in vitro transcription, are well known in the art. It should be noted that oligonucleotide probes are chosen to be complementary to target nucleic acids. Therefore, if an antisense pool of target nucleic acids is provided (as is often the case when target nucleic acids are amplified by in vitro transcription), the oligonucleotide probes should correspond with subsequences of the sense complement. Conversely, if the pool of target nucleic acids is sense, the oligonucleotide array should be complementary (i.e., antisense) to them. Finally, if target nucleic acids are double-stranded, oligonucleotide probes can be sense or antisense.

The present invention involves detecting the hybridization intensity between target nucleic acids and complementary oligonucleotide probes. To accomplish this, target nucleic acids may be attached directly or indirectly with appropriate and detectable labels. Direct labels are detectable labels that are directly attached to or incorporated into target nucleic acids. Indirect labels are attached to polynucleotides after hybridization, often by attaching to a binding moiety that was attached to the target nucleic acids prior to hybridization. Such direct and indirect labels are well known in the art. In a preferred embodiment of the invention, target nucleic acids are detected using the biotin-streptavidin-PE coupling system, where biotin is incorporated into target nucleic acids and hybridization is detected by the binding of streptavidin-PE to biotin.

Target nucleic acids may be labeled before, during or after incubation with an oligonucleotide array. Preferably, the target nucleic acids are labeled before incubation. Labels may be incorporated during the amplification step by using nucleotides that are already labeled (e.g., biotin-coupled dUTP or dCTP) in the reaction. Alternatively, a label may be added directly to the original nucleic acid sample (e.g., mRNA, cDNA) or to the amplification product after the amplification is completed. Means of attaching labels to nucleic acids are well known to those of skill in the art and include, but are not limited to, nick translation, end-labeling, and ligation of target nucleic acids to a nucleic acid linker to join it to a label. Alternatively, several kits specifically designed for isolating and preparing target nucleic acids for microarray analysis are commercially available, including, but not limited to, the GeneChip® IVT Labeling Kit (Affymetrix, Santa Clara, Calif.) and the Bioarray® High Yield® RNA Transcript Labeling Kit with Fluorescein-UTP for Nucleic Acid Arrays (Enzo Life Sciences, Inc., Farmingdale, N.Y.).

Polynucleotides can be fragmented before being labeled with detectable moieties. Exemplary methods for fragmentation include, but are not limited to, heat or ion-mediated hydrolysis.

Oligonucleotide Arrays

Probes suitable for the present invention includes oligonucleotide arrays or other probes that capable of detecting the expression of a plurality of genes (including previously undiscovered genes) by a cell (or cell line), including known cells or cells derived from an unsequenced organism, and to identify genes (including previously undiscovered genes) and related pathways that may be involved with the induction of a particular cell phenotype, e.g., increased and efficient transgene expression.

Oligonucleotide probes used in this invention may be nucleotide polymers or analogs and modified forms thereof such that hybridizing to a pool of target nucleic acids occurs in a sequence specific manner under oligonucleotide array hybridization conditions. As used herein, the term “oligonucleotide array hybridization conditions” refers to the temperature and ionic conditions that are normally used in oligonucleotide array hybridization. In many examples, these conditions include 16-hour hybridization at 45° C., followed by at least three 10-minute washes at room temperature. The hybridization buffer comprises 100 mM MES, 1 M [Na⁺], 20 mM EDTA, and 0.01% Tween 20. The pH of the hybridization buffer can range between 6.5 and 6.7. The wash buffer is 6× SSPET, which contains 0.9 M NaCl, 60 mM NaH2PO4, 6 mM EDTA, and 0.005% Triton X-100. Under more stringent oligonucleotide array hybridization conditions, the wash buffer can contain 100 mM MES, 0.1 M [Na⁺], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

As is known by one of skill in the art, oligonucleotide probes can be of any length. Preferably, oligonucleotide probes suitable for the invention are 20 to 70 nucleotides in length. Most preferably, suitable oligonucleotide probes are 25 nucleotides in length. In one embodiment, the nucleic acid probes of the present invention have relatively high sequence complexity. In many examples, the probes do not contain long stretches of the same nucleotide. In addition, the probes may be designed such that they do not have a high proportion of G or C residues at the 3′ ends. In another embodiment, the probes do not have a 3′ terminal T residue. Depending on the type of assay or detection to be performed, sequences that are predicted to form hairpins or interstrand structures, such as “primer dimers,” can be either included in or excluded from the probe sequences. In many embodiments, each probe employed in the present invention does not contain any ambiguous base.

Oligonucleotide probes are made to be specific for (e.g., complementary to (i.e., capable of hybridizing to)) a template sequence. Any part of a template sequence can be used to prepare probes. Multiple probes, e.g., 5, 10, 15, 20, 25, 30, or more, can be prepared for each template sequence. These multiple probes may or may not overlap each other. Overlap among different probes may be desirable in some assays. In many embodiments, the probes for a template sequence have low sequence identities with other template sequences, or the complements thereof. For instance, each probe for a template sequence can have no more than 70%, 60%, 50% or less sequence identity with other template sequences, or the complements thereof. This reduces the risk of undesired cross-hybridization. Sequence identity can be determined using methods known in the art. These methods include, but are not limited to, BLASTN, FASTA, and FASTDB. The Genetics Computer Group (GCG) program, which is a suite of programs including BLASTN and FASTA, can also be used. Preferable sequences for template sequences include, but are not limited to, consensus sequences, transgene sequences, and control sequences (i.e., sequences used to control or normalize for variation between experiments, samples, stringency requirements, and target nucleic acid preparations). Additionally, any subsequence of consensus, transgene and control sequences can be used as a template sequence.

In one embodiment, only certain regions (i.e., tiling regions) of consensus, transgene and control sequences are used as template sequences for the oligonucleotide probes used in this invention. One of skill in the art will recognize that protocols that may be used in practicing the invention, e.g., in vitro transcription protocols, often result in a bias toward the 3′-ends of target nucleic acids. Consequently, in one embodiment of the invention, the region of the consensus sequence or transgene sequence closest to the 3′-end of a consensus sequence is most often used as a template for oligonucleotide probes. Generally, if a poly-A signal could be identified, the 1400 nucleotides immediately prior to the end of the consensus or transgene sequences are designated as a tiling region. Alternatively, if a poly-A signal could not be identified, only the last 600 nucleotides of the consensus or transgene sequence are designated as a tiling region. However, it should be noted that the invention is not limited to using only these tiling regions within the consensus, transgene and control sequences as templates for the oligonucleotide probes. Indeed, a tiling region may occur anywhere within the consensus, transgene or control sequences. For example, the tiling region of a control sequence may comprise regions from both the 5′ and 3′-ends of the control sequence. In fact, the entire consensus, transgene or control sequence may be used as a template for oligonucleotide probes.

An oligonucleotide array suitable for the invention may include perfect match probes to a plurality of consensus sequences (i.e., consensus sequences for multi-sequence clusters, and consensus sequences for exemplar sequences) identified as described above. The oligonucleotide array suitable for the invention may also include perfect match probes to both consensus and transgene sequences. It will be apparent to one of skill in the art that inclusion of oligonucleotide probes to transgene sequences will be useful when a cell line is genetically engineered to express a recombinant protein encoded by a transgene sequence, and the purpose of the analysis is to confirm expression of the transgene and determine the level of such expression. In those cases where the transgene is linked in a bicistronic mRNA to a downstream ORF, such as dihydrofolate reductase (DHFR), the level of transgene expression may also be determined from the level of expression of the downstream sequence. In another embodiment of the invention, the oligonucleotide array further comprises control probes that normalize the inherent variation between experiments, samples, stringency requirements, and preparations of target nucleic acids. Exemplary compositions of each of these types of control probes is described in U.S. Pat. No. 6,040,138 and in U.S. Publication No. 20060010513, the teachings of both of which are incorporated herein in their entirety by reference.

It is well known to one of skill in the art that two pools of target nucleic acids individually processed from the same sample can hybridize to two separate but identical oligonucleotide arrays with varying results. The varying results between these arrays are attributed to several factors, such as the intensity of the labeled pool of target nucleic acids and incubation conditions. To control for these variations, normalization control probes can be added to the array. Normalization control probes are oligonucleotides exactly complementary to known nucleic acid sequences spiked into the pool of target nucleic acids. Any oligonucleotide sequence may serve as a normalization control probe. For example, the normalization control probes may be created from a template obtained from an organism other than that from which the cell line being analyzed is derived. In one embodiment, an oligonucleotide array to mammalian sequences will contain normalization oligonucleotide probes to the following genes: bioB, bioC, and bioD from the organism Escherichia coli, cre from the organism Bacteriophage PI, and dap from the organism Bacillus subtilis, or subsequences thereof. The signal intensity received from the normalization control probes are then used to normalize the signal intensities from all other probes in the array. Additionally, when the known nucleic acid sequences are spiked into the pool of target nucleic acids at known and different concentrations for each transcript, a standard curve correlating signal intensity with transcript concentration can be generated, and expression levels for all transcripts represented on the array can be quantified (see, e.g., Hill et al. (2001) Genome Biol. 2(12):research0055.1-0055.13).

Due to the naturally differing metabolic states between cells, expression of specific target nucleic acids vary from sample to sample. In addition, target nucleic acids may be more prone to degradation in one pool compared to another pool. Consequently, in another embodiment of the invention, the oligonucleotide array further comprises oligonucleotide probes that are exactly complementary to constitutively expressed genes, or subsequences thereof, that reflect the metabolic state of a cell. Nonlimiting examples of these types of genes are beta-actin, transferrin receptor and glyceraldehyde-3-phosphate dehydrogenase (GAPDH).

In one embodiment of the invention, the pool of target nucleic acids is derived by converting total RNA isolated from the sample into double-stranded cDNA and transcribing the resulting cDNA into complementary RNA (cRNA) using methods described in U.S. Publication No. 20060010513, the teachings of which are incorporated herein in their entirety by reference. The RNA conversion protocol is started at the 3′-end of the RNA transcript, and if the process is not allowed to go to completion (if, for example, the RNA is nicked, etc.) the amount of the 3′-end message compared to the 5′-end message will be greater, resulting in a 3′-bias. Additionally, RNA degradation may start at the 5′-end (Jacobs Anderson et al. (1998) EMBO J. 17:1497-506). The use of these methods suggests that control probes that measure the quality of the processing and the amount of degradation of the sample preferably should be included in the oligonucleotide array. Examples of such control probes are oligonucleotides exactly complementary to 3′- and 5′-ends of constitutively expressed genes, such as beta-actin, transferrin receptor and GAPDH, as mentioned above. The resulting 3′ to 5′ expression ratio of a constitutively expressed gene is then indicative of the quality of processing and the amount of degradation of the sample; i.e., a 3′ to 5′ ratio greater than three (3) indicates either incomplete processing or high RNA degradation (Auer et al. (2003) Nat. Genet. 35:292-93). Consequently, in a preferred embodiment of the invention, the oligonucleotide array includes control probes that are complementary to the 3′- and 5′-ends of constitutively expressed genes.

The quality of the pool of target nucleic acids is not only reflected in the processing and degradation of the target nucleic acids, but also in the origin of the target nucleic acids. Contaminating sequences, such as genomic DNA, may interfere with well-known quantification protocols. Consequently, in a preferred embodiment of the invention, the array further comprises oligonucleotide probes exactly complementary to bacterial genes, ribosomal RNAs, and/or genomic intergenic regions to provide a means to control for the quality of the sample preparation. These probes control for the possibility that the pool of target nucleic acids is contaminated with bacterial DNA, non-mRNA species, and genomic DNA. Such exemplary control sequences are disclosed in U.S. Publication No. 20060010513, the teaching of which are incorporated herein in their entirety by reference.

In a preferred embodiment of the invention, the oligonucleotide array further comprises control mismatch oligonucleotide probes for each perfect match probe. The mismatch probes control for hybridization specificity. Preferably, mismatch control probes are identical to their corresponding perfect match probes with the exception of one or more substituted bases. More preferably, the substitution(s) occurs at a central location on the probe. For example, where a perfect match probe is 25 oligonucleotides in length, a corresponding mismatch probe will have the identical length and sequence except for a single-base substitution at position 13 (e.g., substitution of a thymine for an adenine, an adenine for a thymine, a cytosine for a guanine, or a guanine for a cytosine). The presence of one or more mismatch bases in the mismatch oligonucleotide probe disallows target nucleic acids that bind to complementary perfect match probes to bind to corresponding mismatch control probes under appropriate conditions. Therefore, mismatch oligonucleotide probes indicate whether the incubation conditions are optimal, i.e., whether the stringency being utilized provides for target nucleic acids binding to only exactly complementary probes present in the array.

For each template, a set of perfect match probes exactly complementary to subsequences of consensus, transgene, and/or control sequences (or tiling regions thereof) may be chosen using a variety of strategies. It is known to one of skill in the art that each template can provide for a potentially large number of probes. As is known, apparent probes are sometimes not suitable for inclusion in the array. This can be due to the existence of similar subsequences in other regions of the genome, which causes probes directed to these subsequences to cross-hybridize and give false signals. Another reason some apparent probes may not be suitable for inclusion in the array is because they may form secondary structures that prevent efficient hybridization. Finally, hybridization of target nucleic acids with (or to) an array comprising a large number of probes requires that each of the probes hybridizes to its specific target nucleic acid sequence under the same incubation conditions.

An oligonucleotide array may comprise one perfect match probe for a consensus, transgene, or control sequence, or may comprise a probeset (i.e., more than one perfect match probe) for a consensus, transgene, or control sequence. For example, an oligonucleotide array may comprise 1, 5, 10, 25, 50, 100, or more than 100 different perfect match probes for a consensus, transgene or control sequence. In a preferred embodiment of the invention, the array comprises at least 11-50 different perfect match oligonucleotide probes exactly complementary to subsequences of each consensus and transgene sequence. In an even more preferred embodiment, only the most optimal probeset for each template is included. The suitability of the probes for hybridization can be evaluated using various computer programs. Suitable programs for this purpose include, but are not limited to, LaserGene (DNAStar), Oligo (National Biosciences, Inc.), MacVector (Kodak/IBI), and the standard programs provided by the GCG. Any method or software program known in the art may be used to prepare probes for the template sequences of the present invention. For example, oligonucleotide probes may be generated by using Array Designer, a software package provided by TeleChem International, Inc (Sunnyvale, Calif.). Another exemplary algorithm for choosing optimal probe sets is described in U.S. Pat. No. 6,040,138, the teachings of which are hereby incorporated by reference. Other suitable means to optimize probesets, which will result in a comparable oligonucleotide array, are well known in the art and may be found in, e.g., Lockhart et al. (1996) Nat. Biotechnol. 14:1675-80 and Mei et al. (2003) Proc. Natl. Acad. Sci. USA 100:11237-42.

The oligonucleotide probes of the present invention can be synthesized using a variety of methods. Examples of these methods include, but are not limited to, the use of automated or high throughput DNA synthesizers, such as those provided by Millipore, GeneMachines, and BioAutomation. In many embodiments, the synthesized probes are substantially free of impurities. In many other embodiments, the probes are substantially free of other contaminants that may hinder the desired functions of the probes. The probes can be purified or concentrated using numerous methods, such as reverse phase chromatography, ethanol precipitation, gel filtration, electrophoresis, or any combination thereof.

More detailed information of making an oligonucleotide array suitable for the present invention and exemplary arrays are disclosed in U.S. Publication No. 20060010513, the disclosures of which are hereby incorporated by reference. As described in U.S. Publication No. 20060010513, a CHO chip microarray suitable for the invention includes 122 array quality control sequences (non-CHO), 732 public hamster sequences, 2835 library-derived CHO sequences, and 22 product/process specific sequences. Additional suitable arrays are described in U.S. Pat. No. 6,040,138, the disclosures of which are incorporated by reference.

Incubation of Target Nucleic Acids with an Array to Form a Hybridization Profile

Incubation reactions can be performed in absolute or differential hybridization formats. In the absolute hybridization format, polynucleotides derived from one sample are hybridized to the probes in an oligonucleotide array. Signals detected after the formation of hybridization complexes correlate to the polynucleotide levels in the sample. In the differential hybridization format, polynucleotides derived from two samples are labeled with different labeling moieties. A mixture of these differently labeled polynucleotides is added to an oligonucleotide array. The oligonucleotide array is then examined under conditions in which the emissions from the two different labels are individually detectable. In one embodiment, the fluorophores Cy3 and Cy5 (Amersham Pharmacia Biotech, Piscataway, N.J.) are used as the labeling moieties for the differential hybridization format.

In the present invention, the incubation conditions should be such that target nucleic acids hybridize only to oligonucleotide probes that have a high degree of complementarity. In a preferred embodiment, this is accomplished by incubating the pool of target nucleic acids with an oligonucleotide array under a low stringency condition to ensure hybridization, and then performing washes at successively higher stringencies until the desired level of hybridization specificity is reached. In other embodiments, target nucleic acids are incubated with an array of the invention under stringent or well-known oligonucleotide array hybridization conditions. In many examples, these oligonucleotide array hybridization conditions include 16-hour hybridization at 45 ° C., followed by at least three 10-minute washes at room temperature. The hybridization buffer comprises 100 mM MES, 1 M [Na⁺], 20 mM EDTA, and 0.01% Tween 20. The pH of the hybridization buffer can range between 6.5 and 6.7. The wash buffer is 6× SSPET, which contains 0.9 M NaCl, 60 mM NaH₂PO₄, 6 mM EDTA, and 0.005% Triton X-100. Under more stringent oligonucleotide array hybridization conditions, the wash buffer can contain 100 mM MES, 0.1 M [Na⁺], and 0.01% Tween 20. See also GENECHIP® EXPRESSION ANALYSIS TECHNICAL MANUAL (701021 rev. 3, Affymetrix, Inc. 2002), which is incorporated herein by reference in its entirety.

Differential Gene Expression Profiling Analysis

Methods used to detect the hybridization profile of target nucleic acids with oligonucleotide probes are well known in the art. In particular, means of detecting and recording fluorescence of each individual target nucleic acid-oligonucleotide probe hybrid have been well established and are well known in the art, described in, e.g., U.S. Pat. No. 5,631,734, U.S. Publication No. 20060010513, incorporated herein in their entirety by reference. For example, a confocal microscope can be controlled by a computer to automatically detect the hybridization profile of the entire array. Additionally, as a further nonlimiting example, the microscope can be equipped with a phototransducer attached to a data acquisition system to automatically record the fluorescence signal produced by each individual hybrid.

It will be appreciated by one of skill in the art that evaluation of the hybridization profile is dependent on the composition of the array, i.e., which oligonucleotide probes were included for analysis. For example, where the array includes oligonucleotide probes to consensus sequences only, or consensus sequences and transgene sequences only, (i.e., the array does not include control probes to normalize for variation between experiments, samples, stringency requirements, and preparations of target nucleic acids), the hybridization profile is evaluated by measuring the absolute signal intensity of each location on the array. Alternatively, the mean, trimmed mean (i.e., the mean signal intensity of all probes after 2-5% of the probesets with the lowest and highest signal intensities are removed), or median signal intensity of the array may be scaled to a preset target value to generate a scaling factor, which will subsequently be applied to each probeset on the array to generate a normalized expression value for each gene (see, e.g., Affymetrix (2000) Expression Analysis Technical Manual, pp. A5-14). Conversely, where the array further comprises control oligonucleotide probes, the resulting hybridization profile is evaluated by normalizing the absolute signal intensity of each location occupied by a test oligonucleotide probe by means of mathematical manipulations with the absolute signal intensity of each location occupied by a control oligonucleotide probe. Typical normalization strategies are well known in the art, and are included, for example, in U.S. Pat. No. 6,040,138 and Hill et al. (2001) Genome Biol. 2(12):research0055.1-0055.13.

Signals gathered from oligonucleotide arrays can be analyzed using commercially available software, such as those provide by Affymetrix or Agilent Technologies. Controls, such as for scan sensitivity, probe labeling and cDNA or cRNA quantitation, may be included in the hybridization experiments. The array hybridization signals can be scaled or normalized before being subjected to further analysis. For instance, the hybridization signal for each probe can be normalized to take into account variations in hybridization intensities when more than one array is used under similar test conditions. Signals for individual target nucleic acids hybridized with complementary probes can also be normalized using the intensities derived from internal normalization controls contained on each array. In addition, genes with relatively consistent expression levels across the samples can be used to normalize the expression levels of other genes.

To identify genes that confer or correlate with a desired phenotype or characteristic, a gene expression profile of a sample derived from a test cell line is compared to a control profile derived from a control cell line that has a cell culture phenotype of interest distinct from that of the test cell line and differentially expressed genes are identified. For example, the method for identifying the genes and related pathways involved in cellular productivity may include the following: 1) growing a first sample of a first cell line with a particular cellular productivity and growing a second sample of a second cell line with a distinct cellular productivity; 2) isolating, processing, and hybridizing total RNA from the first sample to a first oligonucleotide array; 3) isolating, processing, and hybridizing total RNA from the second sample to a second oligonucleotide array; and 4) comparing the resulting hybridization profiles to identify the sequences that are differentially expressed between the first and second samples. Similar methods can be used to identify genes involved in other phenotypes.

Typically, each cell line was represented by at least three biological replicates. Programs known in the art, e.g., GeneExpress 2000 (Gene Logic, Gaithersburg, Md.), were used to analyze the presence or absence of a target sequence and to determine its relative expression level in one cohort of samples (e.g., cell line or condition or time point) compared to another sample cohort. A probeset called present in all replicate samples was considered for further analysis. Generally, fold-change values of 1.2-fold, 1.5-fold or greater were considered statistically significant if the p-values were less than or equal to 0.05.

The identification of differentially expressed genes that correlate with one or more particular cell phenotypes (e.g., cell growth rate, peak cell density, sustained high cell viability, maximum cellular productivity, sustained high cellular productivity, ammonium production or consumption, lactate production or consumption, etc.) can lead to the discovery of genes and pathways, including those were previously undiscovered, that regulate or are indicative of the cell phenotypes.

The subsequently identified genes are sequenced and the sequences are blasted against various databases to determine whether they are known genes or unknown genes. If genes are known, pathway analysis can be conducted based on the existing knowledge in the art. Both known and unknown genes are further confirmed or validated by various methods known in the art. For example, the identified genes may be manipulated (e.g., up-regulated or down-regulated) to induce or suppress the particular phenotype by the cells.

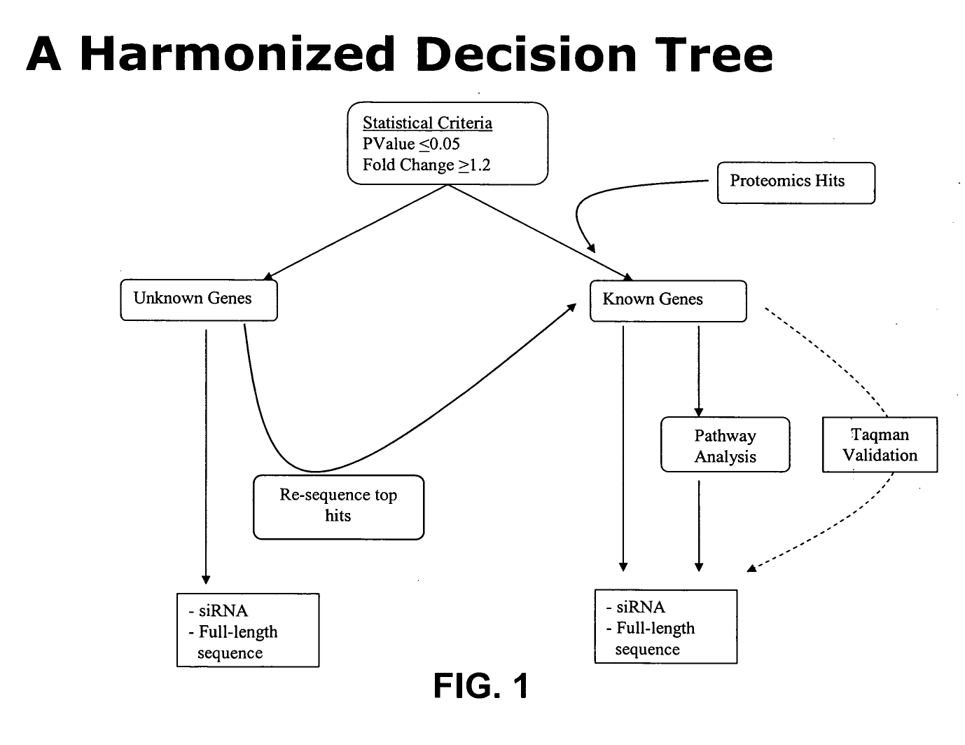

A harmonized decision tree illustrating this process is shown in FIG. 1. More detailed identification and validation steps are further described in the Examples and exemplary differentially expressed genes identified using the method of the invention are shown in Tables 9 through 16.

Differential Protein Expression Profiling Analysis

The present invention also provide methods for identifying differentially expressed proteins by protein expression profiling analysis. Protein expression profiles can be generated by any method permitting the resolution and detection of proteins from a sample from a cell line. Methods with higher resolving power are generally preferred, as increased resolution can permit the analysis of greater numbers of individual proteins, increasing the power and usefulness of the profile. A sample can be pre-treated to remove abundant proteins from a sample, such as by immunodepletion, prior to protein resolution and detection, as the presence of an abundant protein may mask more subtle changes in expression of other proteins, particularly for low-abundance proteins. A sample can also be subjected to one or more procedures to reduce the complexity of the sample. For example, chromatography can be used to fractionate a sample; each fraction would have a reduced complexity, facilitating the analysis of the proteins within the fractions.

Three useful methods for simultaneously resolving and detecting several proteins include array-based methods; mass-spectrometry based methods; and two-dimensional gel electrophoresis based methods.

Protein arrays generally involve a significant number of different protein capture reagents, such as antibodies or antibody variable regions, each immobilized at a different location on a solid support. Such arrays are available, for example, from Sigma-Aldrich as part of their Panorama® line of arrays. The array is exposed to a protein sample and the capture reagents selectively capture the specific protein targets. The captured proteins are detected by detection of a label. For example, the proteins can be labeled before exposure to the array; detection of a label at a particular location on the array indicates the detection of the corresponding protein. If the array is not saturated, the amount of label detected may correlate with the concentration or amount of the protein in the sample. Captured proteins can also be detected by subsequent exposure to a second capture reagent, which can itself be labeled or otherwise detected, as in a sandwich immunoassay format.

Mass spectrometry-based methods include, for example, matrix-assisted laser desorption/ionization (MALDI), Liquid Chromatography/Mass Spectrometry/Mass Spectrometry (LC-MS/MS) and surface enhanced laser desorption/ionization (SELDI) techniques. For example, a protein profile can be generated using electrospray ionization and MALDI. SELDI, as described, for example, in U.S. Pat. No. 6,225,047, incorporates a retention surface on a mass spectrometry chip. A subset of proteins in a protein sample are retained on the surface, reducing the complexity of the mixture. Subsequent time-of-flight mass spectrometry generates a “fingerprint” of the retained proteins.

In methods involving two-dimensional gel electrophoresis, proteins in a sample are generally separated in a first dimension by isoelectric point and in a second dimension by molecular weight during SDS-PAGE. By virtue of the two dimensions of resolution, hundreds or thousands of proteins can be simultaneously resolved and analyzed. The proteins are detected by application of a stain, such as a silver stain, or by the presence of a label on the proteins, such as a Cy2, Cy3, or Cy5 dye. To identify a protein, a gel spot can be cut out and in-gel tryptic digestion performed. The tryptic digest can be analyzed by mass spectrometry, such as MALDI. The resulting mass spectrum of peptides, the peptide mass fingerprint or PMF, is searched against a sequence database. The PMF is compared to the masses of all theoretical tryptic peptides generated in silico by the search program. Programs such as Prospector, Sequest, and MasCot (Matrix Science, Ltd., London, UK) can be used for the database searching. For example, MasCot produces a statistically-based Mowse score indicates if any matches are significant or not. MS/MS can be used to increase the likelihood of getting a database match. CID-MS/MS (collision induced dissociation of tandem MS) of peptides can be used to give a spectrum of fragment ions that contain information about the amino acid sequence. Adding this information to a peptide mass fingerprint allows Mascot to increase the statistical significance of a match. It is also possible in some cases to identify a protein by submitting only a raw MS/MS spectrum of a single peptide.

A recent improvement in comparisons of protein expression profiles involves the use of a mixture of two or more protein samples, each labeled with a different, spectrally-resolvable, charge- and mass-matched dye, such as Cy3 and Cy5. This improvement, called fluorescent 2-dimensional differential in-gel electrophoresis (DIGE), has the advantage that the test and control protein samples are run in the same gel, facilitating the matching of proteins between the two samples and avoiding complications involving non-identical electrophoresis conditions in different gels. The gels are imaged separately and the resulting images can be overlaid directly without further modification. A third spectrally-resolvable dye, such as Cy2, can be used to label a pool of protein samples to serve as an internal control among different gels run in an experiment. Thus, all detectable proteins are included as an internal standard, facilitating comparisons across different gels.

Engineering Cell Lines to Improve Cell Phenotypes

As described above, the present invention provides polynucleotide sequences (or subsequences) of genes or polypeptide sequences (or subsequences) of proteins that are differentially expressed in different cell lines or cell samples with at least one distinct cell phenotype. These sequences are collectively referred to as differential sequences. The differential sequences may be used as targets to effect a cell phenotype, particularly a phenotype characterized by increased and efficient production of a recombinant transgene, increased cell growth rate, high peak cell density, sustained high cell viability, high maximum cellular productivity, sustained high cellular productivity, low ammonium production, and low lactate production, etc.

More particularly, the invention provides each purified and/or isolated polynucleotide or polypeptide sequence referred to in the relevant Tables that is shown to be a suitable target for regulating a CHO cell phenotype, i.e., is differentially expressed by a first CHO cell line compared to a second CHO cell line, herein designated as “differential CHO sequence.” Specifically, as used herein, a differential CHO sequence include a sequence having and/or consisting essentially of a sequence selected from the gene sequences referenced in the Tables, a fragment or a complement thereof. As used herein, a differential CHO sequence also includes a polypeptide sequence selected from the protein sequences referenced in the Tables, or a fragment thereof. As used herein, a differential CHO sequence also includes a polynucleotide sequence encoding a polypeptide sequence selected from the protein sequences referenced in the Tables, a fragment or a complement thereof. A skilled artisan will recognize that the differential CHO sequences of the invention may include novel CHO sequences (as discussed below), known gene sequences that are attributed with a function that is, or was, not obviously involved in transgene expression, and known sequences that previously had no known function but may now be known to function as targets in regulating a CHO cell phenotype.

The present invention contemplates methods and compositions that may be used to alter (i.e., regulate (e.g., enhance, reduce, or modify)) the expression and/or the activity of the genes or proteins corresponding to the differential CHO sequences in a cell or organism. Altered expression of the differential CHO sequences encompassed by the present invention in a cell or organism may be achieved through down-regulating or up-regulating of the corresponding genes or proteins. For example, the differential CHO sequences may be down-regulated by the use of various inhibitory polynucleotides, such as antisense polynucleotides, ribozymes that bind and/or cleave the mRNA transcribed from the genes of the invention, triplex-forming oligonucleotides that target regulatory regions of the genes, and short interfering RNA that causes sequence-specific degradation of target mRNA (e.g., Galderisi et al. (1999) J. Cell. Physiol. 181:251-57; Sioud (2001) Curr. Mol. Med. 1:575-88; Knauert and Glazer (2001) Hum. Mol. Genet. 10:2243-51; Bass (2001) Nature 411:428-29).

The inhibitory antisense or ribozyme polynucleotides suitable for the invention can be complementary to an entire coding strand of a gene of the invention, or to only a portion thereof. Alternatively, inhibitory polynucleotides can be complementary to a noncoding region of the coding strand of a gene of the invention. The inhibitory polynucleotides of the invention can be constructed using chemical synthesis and/or enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Mickelfield (2001) Curr. Med. Chem. 8:1157-70). Alternatively, antisense molecules can be produced biologically using an expression vector into which a polynucleotide of the present invention has been subcloned in an antisense (i.e., reverse) orientation.

In yet another embodiment, the antisense polynucleotide molecule suitable for the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2′-o-methylribonucleotide or a chimeric RNA-DNA analogue, according to techniques that are known in the art.

The inhibitory triplex-forming oligonucleotides (TFOs) suitable for the present invention bind in the major groove of duplex DNA with high specificity and affinity (Knauert and Glazer, supra). Expression of the genes of the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides are short interfering RNA (siRNA) molecules. These siRNA molecules are short (preferably 19-25 nucleotides; most preferably 19 or 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas MRNA (Song et al. (2003) Nat. Med. 9:347-51). In addition, intrathecally delivered siRNA has recently been reported to block pain responses in two models (agonist-induced pain model and neuropathic pain model) in the rat (Dom et al. (2004) Nucleic Acids Res. 32(5):e49).

The siRNA molecules suitable for the present invention can be generated by annealing two complementary single-stranded RNA molecules together (one of which matches a portion of the target mRNA) (Fire et al., U.S. Pat. No. 6,506,559) or through the use of a single hairpin RNA molecule that folds back on itself to produce the requisite double-stranded portion (Yu et al. (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s) containing the sense and antisense siRNA sequences. Recently, reduction of levels of target mRNA in primary human cells, in an efficient and sequence-specific manner, was demonstrated using adenoviral vectors that express hairpin RNAs, which are further processed into siRNAs (Arts et al. (2003) Genome Res. 13:2325-32).

The siRNA molecules targeted to the differential CHO sequences of the present invention can be designed based on criteria well known in the art (e.g., Elbashir et al. (2001) EMBO J. 20:6877-88). For example, the target segment of the target mRNA should begin with AA (preferred), TA, GA, or CA; the GC ratio of the siRNA molecule should be 45-55%; the siRNA molecule should not contain three of the same nucleotides in a row; the siRNA molecule should not contain seven mixed G/Cs in a row; and the target segment should be in the ORF region of the target mRNA and should be at least 75 bp after the initiation ATG and at least 75 bp before the stop codon. siRNA molecules targeted to the polynucleotides of the present invention can be designed by one of ordinary skill in the art using the aforementioned criteria or other known criteria.

Down-regulation of the genes or proteins of the present invention in a cell or organism may also be achieved through the creation of cells or organisms whose endogenous genes corresponding to the differential CHO sequences of the present invention have been disrupted through insertion of extraneous polynucleotides sequences (i.e., a knockout cell or organism). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout cells include homologous recombination and are well known in the art (e.g., Wolfer et al. (2002) Trends Neurosci. 25:336-40).

The expression or activity of the CHO differential sequences may also be altered by up-regulating the genes or proteins corresponding to the CHO differential sequences of the invention. Up-regulation includes providing an exogenous nucleic acid (e.g., an over-expression construct) encoding a protein or gene of interest or a variant retaining its activity or providing a factor or a molecule indirectly enhancing the protein activity. The variant generally shares common structural features with the protein or gene of interest and should retain the activity permitting the improved cellular phenotype. The variant may correspond to a homolog from another species (e.g. a rodent homolog; a primate homolog, such as a human homolog; an other mammalian homolog; or a more distant homolog retaining sequence conservation sufficient to convey the desired effect on cellular phenotype). In some cases, the variant may retain at least 70%, at least 80%, at least 90%, or at least 95% sequence identity with the CHO sequence or with a known homolog. In certain embodiments, the variant is a nucleic acid molecule that hybridizes under stringent conditions to the CHO nucleic acid sequence or to the nucleic acid sequence of a known homolog.

For example, the isolated polynucleotides corresponding to the differential CHO sequences of the present invention may be operably linked to an expression control sequence such as the pMT2 and pED expression vectors for recombinant production of differentially expressed genes or proteins of the invention. General methods of expressing recombinant proteins are well known in the art.

The expression or activity of the differentially expressed genes or proteins of the present invention may also be altered by exogenous agents, small molecules, pharmaceutical compounds, or other factors that may be directly or indirectly modulating the activity of the genes or proteins of the present invention. As a result, these agents, small molecules, pharmaceutical compounds, or other factors may be used to regulate the phenotype of CHO cells, e.g., increased production of a recombinant transgene, increased cell growth rate, high peak cell density, sustained high cell viability, high maximum cellular productivity, sustained high cellular productivity, low ammonium production, and low lactate production, etc.

Any combinations of the methods of altering gene or protein expression described above are within the scope of the invention. Any combination of genes or proteins affecting different cell phenotypes can be modulated based on the methods described herein and are within the scope of the invention.

Novel Genes or Proteins

As described above, the present invention provides differential sequences including sequences newly discovered to be expressed by CHO cells. Accordingly, the present invention provides novel isolated and/or purified polynucleotides that are at least part of previously undiscovered genes. Exemplary novel polynucleotide sequences (or subsequences) of genes that are newly discovered expressed by CHO cells are illustrated in Tables 9, 13, and 15. The present invention also provides isolated and/or purified polypeptides that are at least part of previously undiscovered proteins. Exemplary novel polypeptide sequences (or subsequences) of proteins that are newly discovered expressed by CHO cells are illustrated in Tables 2 and 4. The present invention also provides novel polynucleotides encoding the polypeptides sequences as illustrated in Tables 2 and 4.

Thus, the invention provides each purified and/or isolated polynucleotide sequence selected from Tables 9, 13, and 15 that is, or is part of, a previously undiscovered gene (i.e., a gene that had not been sequenced and/or shown to be expressed by CHO cells) and is verifiably expressed by CHO cells. Alternatively, the invention provides each purified and/or isolated polypeptide sequence selected from Tables 2 and 4 that is, or is part of, a previously undiscovered protein (i.e., a protein that had not been sequenced and/or shown to be expressed by CHO cells) and is verifiably expressed by CHO cells. The invention also provides isolated and/or purified polynucleotide sequence encoding each polypeptides sequence selected from Tables 2 and 4. These sequences are herein collectively designated as “novel CHO sequences.” Preferred polynucleotide sequences of the invention include DNA sequences including genomic and cDNA sequences and chemically synthesized DNA sequences, RNA sequences, or other modified nucleic acid sequences. Preferred polypeptide sequences of the invention include amino acid sequences or modified amino acid sequences.

It is part of the invention to provide inhibitory polynucleotides to each novel CHO sequence as described above. Polynucleotides of the present invention also include polynucleotides that hybridize under stringent conditions to novel CHO sequences, or complements thereof, and/or encode polypeptides that retain substantial biological activity of polypeptides encoded by novel CHO sequences of the invention. Polynucleotides of the present invention also include continuous portions of novel CHO sequences comprising at least 21 consecutive nucleotides.

Polynucleotides of the present invention also include polynucleotides that encode any of the amino acid sequences encoded by the polynucleotides as described above, or continuous portions thereof, and that differ from the polynucleotides described above only due to the well-known degeneracy of the genetic code.

The isolated polynucleotides of the present invention may be used as hybridization probes (e.g., as an oligonucleotide array, as described above) and primers to identify and isolate nucleic acids having sequences identical to, or similar to, those encoding the disclosed polynucleotides. Hybridization methods for identifying and isolating nucleic acids include polymerase chain reaction (PCR), Southern hybridization, and Northern hybridization, and are well known to those skilled in the art.

Hybridization reactions can be performed under conditions of different stringencies. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Stringency Conditions

| | | | Hybridization | |
|---|---|---|---|---|
| Stringency | Poly-nucleotide | | Temperature and | Wash Temp. |
| Condition | Hybrid | Hybrid Length (bp)¹ | Buffer^{H} | and Buffer^{H} |
| A | DNA:DNA | >50 | 65° C.; 1xSSC -or- | 65° C.; 0.3xSSC |
| | | | 42° C.; 1xSSC, 50% | |
| | | | formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% | |
| | | | formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% | |
| | | | formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}*; 2xSSC | T_{L}*; 2xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ^{H}SSPE (1x SSPE is 0.15M NaCl, 10 mM NaH₂PO₄, and 1.25 mM EDTA, pH 7.4) can be substituted for SSC (1x SSC is 0.15M NaCl and 15 mM sodium citrate) in the hybridization and wash buffers. | | | | |
| T_{B}* − T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, | | | | |
| #Tₘ(° C.) = 81.5 + 16.6(log₁₀[Na⁺]) + 0.41(% G + C) − (600/N), where N is the number of bases in the hybrid, and [Na⁺] is the molar concentration of sodium ions in the hybridization buffer ([Na⁺] for 1x SSC = 0.165 M). | | | | |

Generally, and as stated above, the isolated polynucleotides of the present invention may also be used as hybridization probes and primers to identify and isolate DNAs homologous to the disclosed polynucleotides. These homologs are polynucleotides isolated from different species than those of the disclosed polynucleotides, or within the same species, but with significant sequence similarity to the disclosed polynucleotides. Preferably, polynucleotide homologs have at least 60% sequence identity (more preferably, at least 75% identity; most preferably, at least 90% identity) with the disclosed polynucleotides. Preferably, homologs of the disclosed polynucleotides are those isolated from mammalian species.

The isolated polynucleotides of the present invention may also be used as hybridization probes and primers to identify cells and tissues that express the polynucleotides of the present invention and the conditions under which they are expressed.

The present invention also contemplates recombinantly express the proteins or polypeptides encoded by the novel CHO sequences. A number of cell types may act as suitable host cells for recombinant expression of the polypeptides encoded by the novel CHO sequences of the invention. Mammalian host cells include, but are not limited to, e.g., COS cells, CHO cells, 293 cells, A431 cells, 3T3 cells, CV-1 cells, HeLa cells, L cells, BHK21 cells, HL-60 cells, U937 cells, HEK cells, PerC6 cells, Jurkat cells, normal diploid cells, cell strains derived from in vitro culture of primary tissue, and primary explants.

Alternatively, it may be possible to recombinantly produce the polypeptides encoded by the novel CHO sequences of the present invention in lower eukaryotes such as yeast or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides are made in yeast or bacteria, it may be necessary to modify them by, e.g., phosphorylation or glycosylation of appropriate sites, in order to obtain functionality. Such covalent attachments may be accomplished using well-known chemical or enzymatic methods.

The polypeptides encoded by polynucleotides of the present invention may also be recombinantly produced by operably linking the isolated novel CHO sequences of the present invention to suitable control sequences in one or more insect expression vectors, such as baculovirus vectors, and employing an insect cell expression system. Materials and methods for baculovirus/Sf9 expression systems are commercially available in kit form (e.g., the MaxBac® kit, Invitrogen, Carlsbad, Calif.).

Following recombinant expression in the appropriate host cells, the polypeptides encoded by polynucleotides of the present invention may then be purified from culture medium or cell extracts using known purification processes, such as gel filtration and ion exchange chromatography. Purification may also include affinity chromatography with agents known to bind the polypeptides encoded by the polynucleotides of the present invention. These purification processes may also be used to purify the polypeptides from natural sources.

Alternatively, the polypeptides encoded by the novel CHO sequences of the present invention may also be recombinantly expressed in a form that facilitates purification. For example, the polypeptides may be expressed as fusions with proteins such as maltose-binding protein (MBP), glutathione-S-transferase (GST), or thioredoxin (TRX). Kits for expression and purification of such fusion proteins are commercially available from New England BioLabs (Beverly, Mass.), Pharmacia (Piscataway, N.J.), and Invitrogen (Carlsbad, Calif.), respectively. The polypeptides encoded by polynucleotides of the present invention can also be tagged with a small epitope and subsequently identified or purified using a specific antibody to the epitope. A preferred epitope is the FLAG epitope, which is commercially available from Eastman Kodak (New Haven, Conn.).

The polypeptides encoded by the novel CHO sequences of the present invention may also be produced by known conventional chemical synthesis. Methods for chemically synthesizing the polypeptides encoded by the novel CHO sequences of the present invention are well known to those skilled in the art. Such chemically synthetic polypeptides may possess biological properties in common with the natural, purified polypeptides, and thus may be employed as biologically active or immunological substitutes for the natural polypeptides.

It should be understood that the above-described embodiments and the following examples are given by way of illustration, not limitation. Various changes and modifications within the scope of the present invention will become apparent to those skilled in the art from the present description.

### EXAMPLES

### Example 1

### Cell culture

Cells were cultured in serum-free suspension culture in two basic formats, under two basic conditions. One format was small scale, shake flask culture in which cells were cultured in less than 100 ml in a vented tissue culture flask, rotated on an orbiting shaker in a CO₂ incubator. The second format was in bench top bioreactors, 2L or less working volume, controlled for pH, nutrients, dissolved oxygen, and temperature. The two basic culture conditions were ordinary passage conditions of 37C, or fed batch culture conditions. In a basic fed batch culture, the cells are grown for a longer period of time, and shifted to a lower temperature in order to prolong cell viability and extend to the productive phase of the culture.

### Example 2

### Classification of CHO Cell Cultures

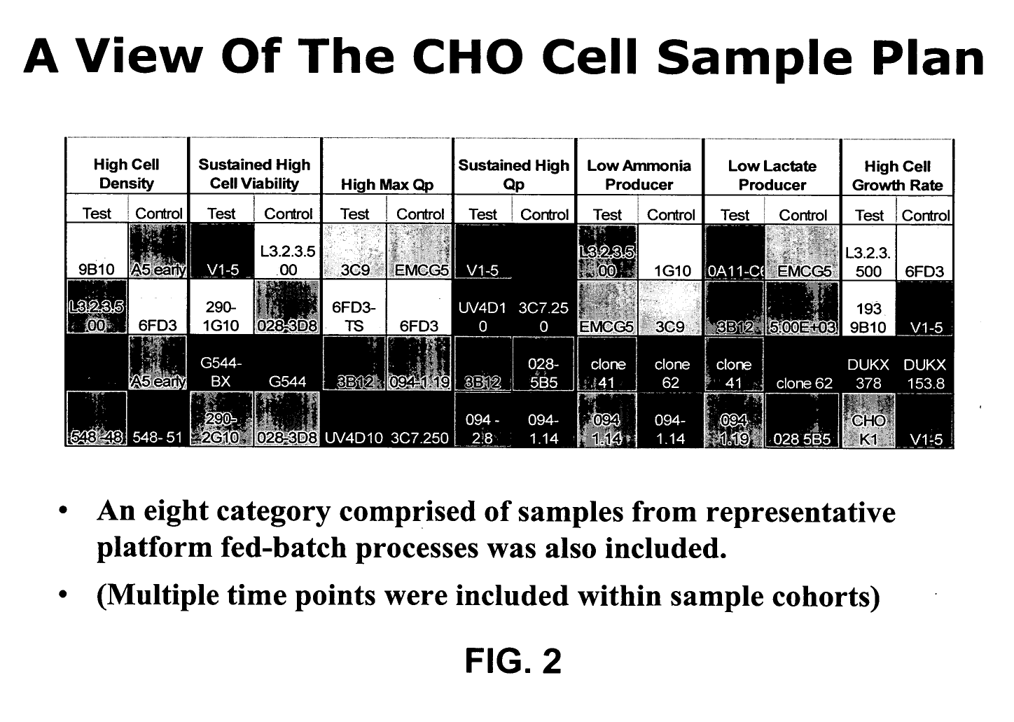

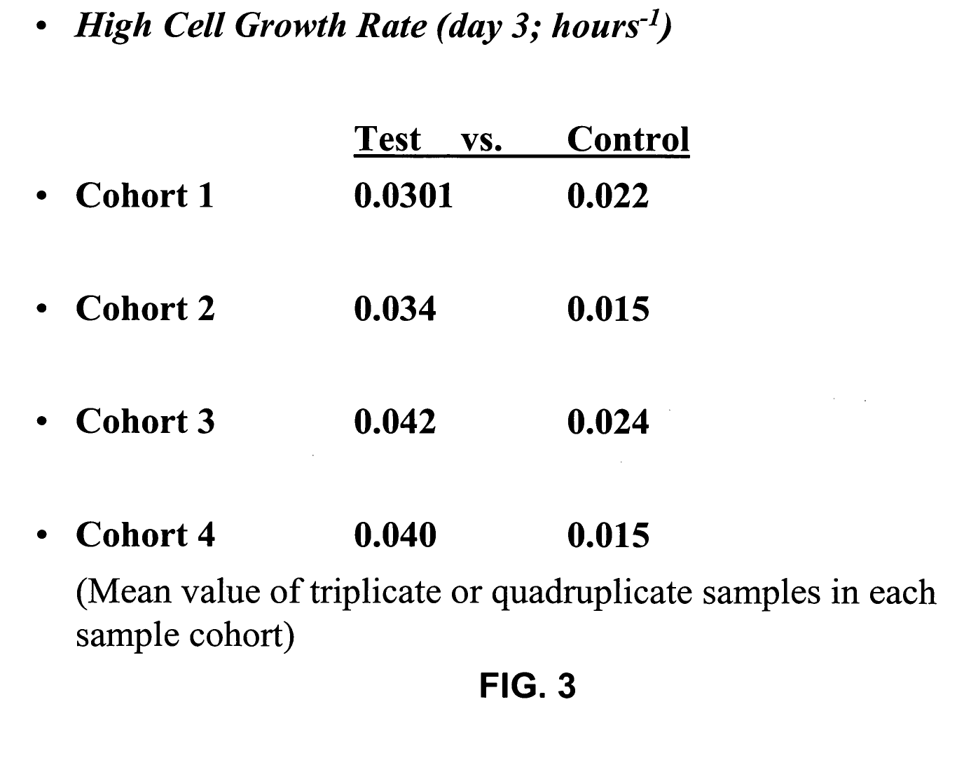

CHO cell lines were categorized based on each of the following phenotypes useful for highly productive fed-batch cell culture processes: high cell growth rate, high peak cell density, sustained high cell viability, high maximum cellular productivity, sustained high cellular productivity, low ammonium production, and low lactate production. A cell sample matrix was generated in which the phenotypic categories were populated with the appropriate CHO cell samples taken from shake flask and benchtop bioreactor cultures and included 375 individual samples (including biological triplicates or quadruplicates) and 29 different rCHO lines expressing monoclonal antibodies, cytokines, coagulation factors and Fc:receptor fusion molecules. An exemplary portion of the cell sample matrix is depicted in FIG. 2, in which the abbreviation Qp is used for cellular productivity. An exemplary phenotypic comparison between test cell lines and control cell lines for the “high cell growth rate” phenotype is depicted in FIG. 3.

### Example 3

### Detection of Differentially Expressed Proteins

Method

Cells were harvested and subjected to standard lysis in 7 M urea, 2 M thiourea, 4% CHAPS, 30 mM Tris, 5 mM magnesium acetate at pH 8.5. 150 μg aliquots of the lysates were analyzed by two-dimensional gel electrophoresis to confirm sample quality using 18 cm immobilized pH gradient isoelectric focusing gradient strips, pH 4-7. The strips were rehydrated overnight with 340 μl of buffer per strip. Samples were loaded at the cathodic end of the strip and subjected to 500 V for 1 hour, 1000 V for 1 hour, and 8000 V for 4 hours and stored at −80° C. until the second dimension on 12.5% acrylamide gels. Electrophoresis in the second dimension was performed at 1.5 W per gel for 30 minutes and then a total of 100 W for 5 hours for a Dalt 6 run of 6 large format gels. Proteins were visualized by silver staining to confirm the quality of the proteins in the lysate.

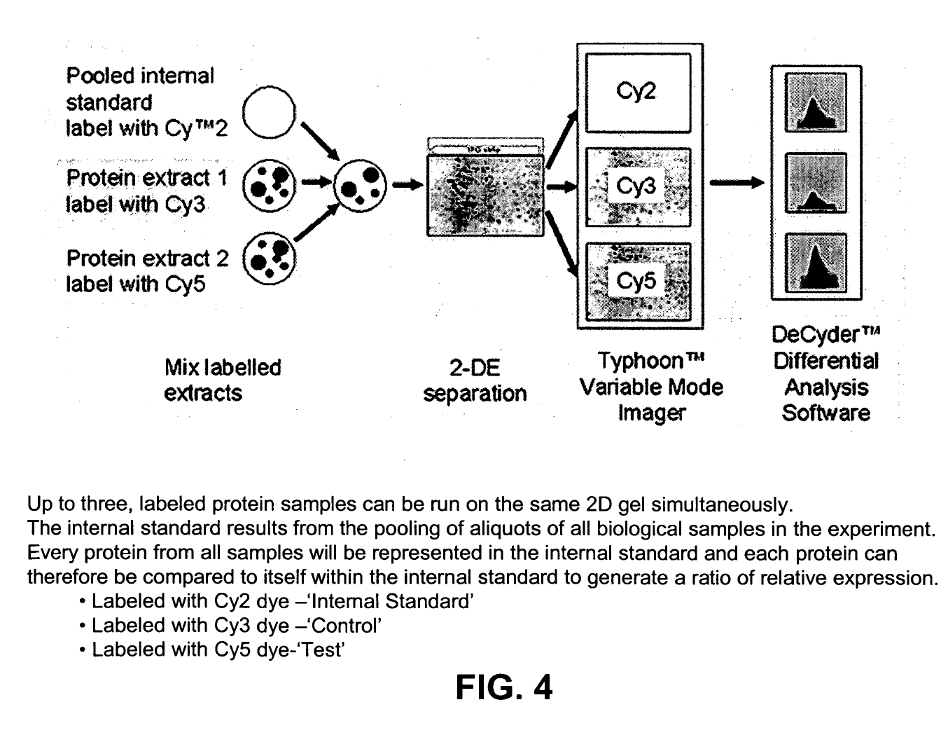

Aliquots of the original lysates were then labeled with fluorescent dyes in preparation for fluorescent 2-dimensional differential in-gel electrophoresis (DIGE), an overview of which is shown in FIG. 4. Each comparison of cell cultures was performed four times using duplicate gels for a total of 8 DIGE gels per experiment, using 50 μg each of Cy2-, Cy3-, and Cy5-labeled cell lysates per gel. All cell lysates used in an experiment were pooled and labeled with Cy2 to serve as an internal standard. The control cell lysate was labeled with Cy3 and the test cell lysate is labeled with Cy5. Labeling was performed on ice in the dark for 30 minutes, followed by a 10 minute quenching of the reaction using 10 mM lysine on ice in the dark. The Cy2-, Cy3-, and Cy5-labeled lysates were then pooled and mixed with 2× sample buffer for 15 minutes in the dark on ice.

The samples were applied to immobilized pH gradient isoelectric focusing strips. The strips were rehydrated overnight for about 20 hours. Samples were loaded at the cathodic end of the strip and subjected to 300V/3 hr/G, 600V/3 hr/S&H, 1000V/3 hr/G, 8000V/3 hr/G, 8000V/4 hr/S&H, and 500V/12 hr/S&H. One hour before SDS-PAGE, the strips were subjected to 8000V for one hour. The strips were equilibrated for 15 minutes in SDS buffer+1% DTT and for 15 minutes in SDS buffer+2.5% iodoacetamide. The strips were applied to polyacrylamide gels and overlaid with agarose. Electrophoresis through the gels was performed at 1.5 W/gel at 10° C. for about 18 hours on a Dalt 12 using 12 large format gels. The gels were scanned on a Typhoon™ 9400 scanner with a variable mode imager; cropped; and imported into DeCyder™ software. Differentially regulated proteins were identified using biological variance analysis (BVA). These proteins were matched to a preparative gel loaded with 400 μg of protein and stained with ruthenium. From the preparative gel, an Ettan Spot Picker was used to pick proteins identified by DIGE as differentially regulated. An Ettan Digestor was used to digest the individual proteins with an overnight trypsin incubation. The resulting peptides were analyzed by mass spectrometry. MALDI is used, particularly for highly abundant samples on gels, for peptide mass fingerprinting.

For lower abundance samples, LC-MS/MS using an MDLC LTQ machine is used. Tryptically digested samples from 2D gel spots were resuspended in 20 μL of LC-MS grade water containing 0.1% TFA and analysed by one-dimensional LC-MS using the Ettan™ MDLC system (GE Healthcare) in high-throughput configuration directly connected to a Finnigan™ LTQ™ (Thermo Electron). Samples were concentrated and desalted on RPC trap columns (Zorbax™ 300SB C18, 0.3 mm×5 mm, Agilent Technologies) and the peptides were separated on a nano-RPC column (Zorbax™ 300SB C18, 0.075 mm×100 mm, Agilent Technologies) using a linear acetonitrile gradient from 0-65% Acetonitrile (Riedel-de Haën LC-MS grade) over 60 minutes directly into the LTQ via a 10 μm nanoESI emitter (Presearch FS360-20-10-CE-20). The LTQ ion trap mass spectrometer was used for MS/MS. A scan time of ˜0.15 s (one microscans with a maximum ion injection time of 10 ms) over an m/z range of 300-2000 was used followed by MS/MS analysis of the 3 most abundant peaks from each scan which were then excluded for the next 60 seconds followed by MS/MS of the next three abundant peaks which in turn were excluded for 60 seconds and so on. A “collision energy” setting of 35% was applied for ion fragmentation and dynamic exclusion was used to discriminate against previously analysed ions (data dependent analysis).

All buffers used for nanoLC separations contained 0.1% Formic Acid (Fluka) as the ion pairing reagent. Full scan mass spectra were recorded in profile mode and tandem mass spectra in centroid mode. The peptides were identified using the information in the tandem mass spectra by searching against SWISS PROT database using SEQUEST™. An Xcorr value of >1.5 for singly charged peptides, >2.0 for doubly charged peptide and >2.5 for triply charged peptides was used as statistical cut-off.

Markers for Maximum Cellular Productivity

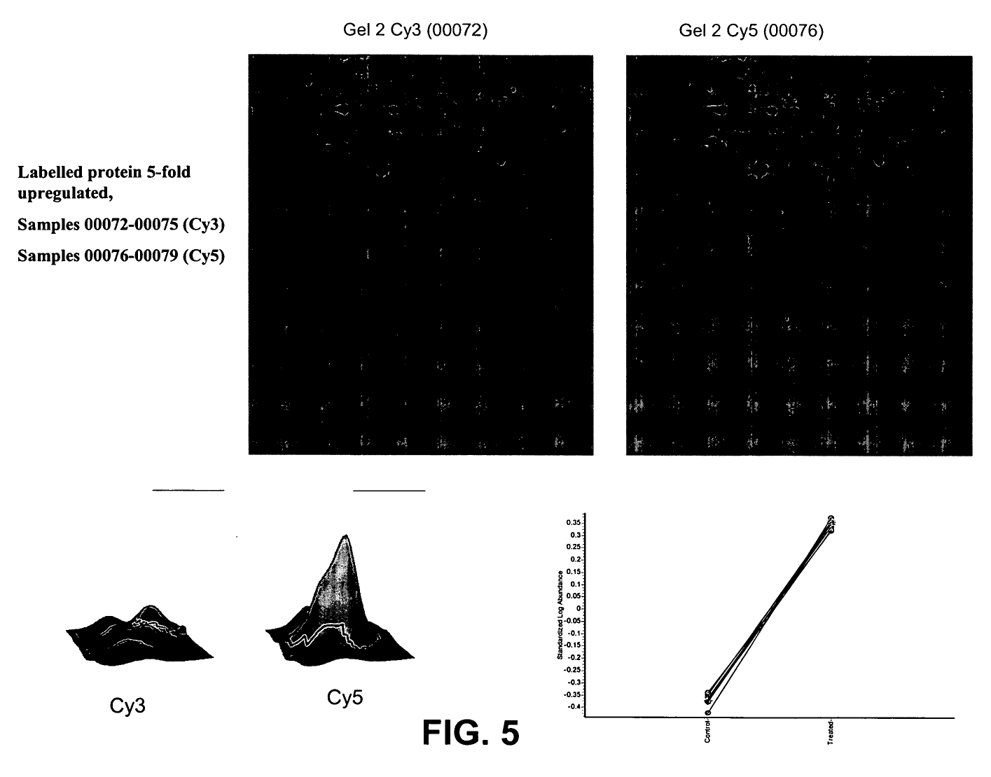

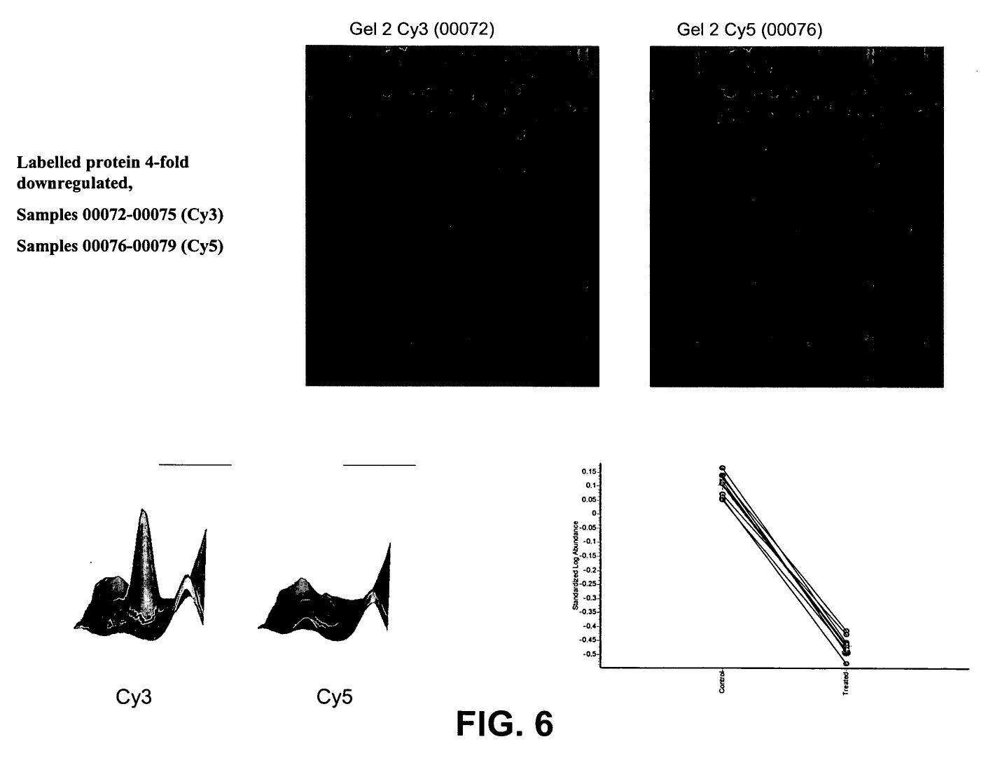

The protein expression profile of four cultures of a cell line overexpressing PACE (furin preproprotein), having a high maximum cellular productivity, was compared to the protein expression profile of four cultures of a control cell line. Approximately 2000 proteins were matched across all 8 gel experiments (involving a total of 24 images). To be considered as a differentially-expressed protein in the DeCyder analysis, a protein must have been identified in all 24; have demonstrated at least a 1.5-fold up- or down-regulation; and have demonstrated a T-test score less than 0.05. 188 proteins were identified as differentially regulated, most with highly significant T-test scores, including several low abundance proteins. FIG. 5 depicts the Cy3 and Cy5 staining patterns on an exemplary gel. A protein that appears to be 5-fold upregulated in the Cy5-labeled test cell extract is outlined in the Figure; graphical depictions of the relative abundance of the protein in the Cy5-labeled test cell extract are also shown. A protein that appears to be 4-fold downregulated in the Cy5-labeled test cell extract is outlined in FIG. 6 and graphical depictions analogous to those in the previous Figure are shown.

Tables 2 and 3 list several of the spots identified as differentially expressed in the high maximal cellular productivity cell line. For each of the spots listed in the tables, MALDI sequence analysis identified one or two corresponding amino acid sequences. The tables provide, for each spot number, the fold difference in protein levels between the test and control samples, labeled as “Average Ratio”; proteins whose levels are reduced in the test samples are indicated with a negative sign. The tables also provide the p-value that the differences in expression would be the result of random chance and the protein name and accession number corresponding to any identified amino acid sequence. In the MALDI sequence analysis, the molecular weights of the trypsin fragments were compared to predicted molecular weights of trypsin fragments of known sequences. In some cases, in this sequence analysis and in other peptide sequence analyses included in this application, the detected molecular weights are indicative of detection of a modified form of a peptide, such as where cysteine has been modified with iodacetamide, or where methionine has been partially oxidized. It is understood that this is not necessarily reflective of the initial state of the peptide in the context of the protein in the cell or the cellular milieu. Accordingly, the peptide sequences provided in the sequence listing reflect the unmodified forms of the peptide, and cells engineered to have desirable cellular phenotypes will, in some embodiments, be engineered to regulate genes expressing an amino acid sequence comprising one or more of the peptides.

In the tables, “% coverage” refers to the percentage of the total length of a database sequence for which corresponding trypsin fragments were detected in the experiment. pI and M_{R} refer to the apparent isoelectric point and apparent molecular weight of the protein spot. For some proteins, putative protein functions are also provided in the table.

TABLE 2
High Max Qp Prot Proteins Identified as Novel Homologs of Non-Hamster Proteins

| | AverageRatio | | | | | | | | |
|---|---|---|---|---|---|---|---|---|---|
| Spot no. | (Test/Control) | p-value | gi accession no. | Protein name | % coverage | pI | Mr | Function | Species |
| 912 | 1.9 | 3.2 × 10−12 | gi\|49645 | Protein disulfide-isomerase A6 | 18.2 | 5 | 48.54 | Catalyzes the re-arrangement of S—S | Mesocricetus |
| | | | and gi\|62296810 | precursor (Protein disulfide isomerase | | | | bonds in proteins/protein folding | auratus and Rattus |
| | | | | P5) (Calcium-binding protein 1) | | | | | norvegicus |
| | | | | (CaBP1) | | | | | |
| 105 | −1.87 | 1.4 × 10−6 | gi\|14250200 | vinculin | 11.4 | 5.8 | 117.3 | | Mus musculus |
| 114 | 1.59 | 5.9 × 10−5 | gi\|24025637 | heat shock protein 4 | 11.9 | 5.1 | 94.84 | protein folding | Rattus norvegicus |
| 310 | 1.63 | 1.1 × 10−9 | gi\|51948378 | minichromosome maintenance protein 7 | 13.9 | 5.9 | 81.67 | | Rattus norvegicus |
| 326 | 8.28 | 2 × 10−12 | gi\|4505579 | furin preproprotein (PACE) | 7.9 | 6 | 87.92 | Endoprotease/precursor | Homo sapiens |
| | | | | | | | | processing activity (release of | |
| | | | | | | | | mature proteins from pro-proteins) | |
| 381 | 1.5 | 8.90E−09 | gi\|31981237 | thimet oligopeptidase 1 | 18.6 | 5.7 | 78.81 | | Mus musculus |
| 467 | −1.55 | 1.00E−09 | gi\|31981769 | glycerol phosphate dehydrogenase 2, | 8.8 | 6.3 | 81.48 | | Mus musculus |
| | | | | mitochondrial | | | | | |
| 585 | −1.97 | 7.7 × 10−10 | gi\|1915913 | Ulip2 protein | 23.8 | 6 | 62.55 | | Mus musculus |
| 627 | 1.58 | 5.8 × 10−9 | gi\|40018616 | chaperonin containing TCP1, subunit 3 (gamma) | 18 | 6.2 | 61.2 | Protein folding | Rattus norvegicus |
| 662 | −3.66 | 3.6 × 10−15 | gi\|34853001 | PREDICTED: similar to UDP-N- | 14.8 | 5.4 | 57.16 | | Rattus norvegicus |
| | | | | acteylglucosamine pyrophosphorylase | | | | | |
| | | | | 1-like 1 | | | | | |
| 759 | −1.77 | 1.4 × 10−9 | gi\|52353955 | 3-phosphoglycerate dehydrogenase | 14.8 | 6.1 | 57.37 | | Mus musculus |
| 831 | −1.55 | 7.2 × 10−8 | gi\|1708472 | Inosine-5′-monophosphate | 17.5 | 6.3 | 55.62 | | Mus musculus |
| | | | | dehydrogenase 1 (IMP dehydrogenase | | | | | |
| | | | | 1) (IMPDH-I) (IMPD 1) | | | | | |
| 899 | 1.58 | 4.4 × 10−6 | gi\|53237082 | eukaryotic translation initiation factor 3, subunit 5 (epsilon) | 15.2 | 5.2 | 38.09 | Translation initiation | Mus musculus |
| 968 | 1.62 | 6 × 10−9 | gi\|14010837 | NSFL1 (p97) cofactor (p47) | 42.7 | 5 | 40.66 | | Rattus norvegicus |
| 1082 | −2.5 | 6.9 × 10−13 | gi\|12842724 | unnamed protein product | 16 | 7.7 | 44.45 | | Mus musculus |
| 1126 | 1.52 | 0.00012 | gi\|62296810 | Protein disulfide-isomerase A6 | 12.1 | 5 | 48.54 | Catalyses the re-arrangement of S—S | Rattus norvegicus |
| | | | | precursor (Protein disulfide isomerase | | | | bonds in proteins/protein folding | |
| | | | | P5) (Calcium-binding protein 1) | | | | | |
| | | | | (CaBP1) | | | | | |
| 1129 | −2.12 | 3.1 × 10−13 | gi\|62296810 | Capg protein | 20.6 | 6.5 | 39.04 | Caps actin filaments/intermediate | Rattus norvegicus |
| | | | | | | | | filament assembly | |
| 1159 | 1.61 | 1.5 × 10−6 | gi\|21312564 | calponin 3, acidic | 29.8 | 5.7 | 36.51 | | Mus musculus |
| 1221 | 1.59 | 7.4 × 10−5 | gi\|54114937 | Eno1 protein (enolase) | 15.3 | 7.8 | 50.18 | | Mus musculus |
| 1265 | 1.66 | 2.6 × 10−6 | gi\|34870516 | PREDICTED: similar to RIKEN cDNA | 24 | 5.2 | 35.04 | | Rattus norvegicus |
| 1274 | 1.72 | 1.8 × 10−3 | gi\|3041728 | 60S acidic ribosomal protein P0 (L10E) | 29.8 | 5.7 | 36.51 | | Bos taurus |
| 1295 | 1.57 | 1.5 × 10−11 | gi\|51262090 | Eef1d protein (eukaryotic translation | 24.2 | 4.9 | 31.39 | Protein translation | Mus musculus |
| | | | | elongation factor 1-delta) | | | | | |
| 1326 | −1.73 | 2.8 × 10−10 | gi\|47169319 | Chain D, Structure Of Pitp-Alpha | 17.8 | 6.1 | 30.97 | | Homo sapiens |
| | | | | Complexed To Phosphatidylinositol | | | | | |
| 1341 | −1.51 | 8.0 × 10−10 | gi\|15100179 | malate dehydrogenase (soluble) | 31.5 | 7.7 | 38.91 | | Rattus norvegicus |
| 1352 | 1.55 | 4.5 × 10−12 | gi\|10442752 | eukaryotic translation elongation factor | 21.4 | 4.9 | 31.39 | Protein translation | Mus musculus |
| | | | | 1-delta | | | | | |
| 1359 | 1.59 | 2 × 10−15 | gi\|10442752 | eukaryotic translation elongation factor | 29.5 | 4.9 | 31.39 | Protein translation | Mus musculus |
| | | | | 1-delta | | | | | |
| 1412 | −1.62 | 3.3 × 10−11 | gi\|388923 | purine-nucleoside phosphorylase | 23.9 | 6.5 | 32.57 | | Mus spretus |
| 1426 | 1.78 | 1.1 × 10−3 | gi\|73968592 | PREDICTED: similar to cyclin- | 38.1 | 6.3 | 27.56 | | Canis familiaris |
| | | | | dependent kinase 4 isoform 3 | | | | | |
| 1454 | 1.73 | 3.6 × 10−10 | gi\|18044897 | Tyms protein (thymidylate synthase) | 19.2 | 5.9 | 35.2 | | Mus musculus |
| 1525 | 2.1 | 2.1 × 10−12 | gi\|13928824 | tyrosine 3-monooxygenase/tryptophan | 39.2 | 4.6 | 29.34 | | Rattus norvegicus |
| | | | | 5-monooxygenase activation protein | | | | | |
| 1525 | 2.1 | 2.1 × 10−12 | gi\|73961101 | Tropomyosin | 41.9 | 4.7 | 26.63 | | Canis familiaris |
| 1577 | 1.53 | 8 × 10−11 | gi\|53733577 | Rho GDP dissociation inhibitor (GDI) | 33.3 | 5.1 | 23.45 | Signal transduction/cell motility/ | Rattus norvegicus |
| | | | and | alpha | | | | cytoskeletal activity | and Mus musculus |
| | | | gi\|31982030 | | | | | | |
| 1678 | 3.48 | 6.2 × 10−12 | gi\|14010865 | heat shock 27 kDa protein | 35.1 | 6.1 | 22.86 | Chaperone | Rattus norvegicus |
| 1685 | 3.74 | 5.3 × 10−14 | gi\|14010865 | heat shock 27 kDa protein | 26.8 | 6.1 | 22.86 | Chaperone | Rattus norvegicus |
| 1724 | −3.46 | 4.1 × 10−18 | gi\|34879492 | similar to Translationally controlled | 13.9 | 5.2 | 19.09 | | Rattus norvegicus |
| | | | | tumor protein (TCTP) (p23) (21 kDa | | | | | |
| | | | | polypeptide) (p21) (Lens epithelial | | | | | |
| | | | | protein) | | | | | |
| 1814 | −1.72 | 2.3 × 10−10 | gi\|817935 | adenine phophoribosyltransferase | 38.3 | 5.7 | 19.61 | | Cricetulus |
| | | | | | | | | | longicaudatus |
| 2000 | −1.8 | 1.3 × 10−8 | gi\|1083180 | galectin-1 | 37.8 | 5.5 | 15.13 | Unknown function in CHO, may be | Cricetulus griseus |
| | | | | | | | | involved in cell proliferation. In | |
| | | | | | | | | humans, may have role in | |
| | | | | | | | | apoptosis & cell differentiation | |
| 108 | 1.67 | 2.90E−06 | gi\|62087882 | heat shock 70 kDa protein 4 isoform a | 9.7 | 0 | 5.4 | | Homo sapiens |
| 123 | 1.53 | 0.0011 | gi\|26522952 | alanyl trna synthetase | 17.8 | 0 | 5.3 | | Mesocricetus |
| | | | | | | | | | auratus |
| 256 | −1.87 | 1.70E−07 | gi\|56605726 | eukaryotic translation initiation factor | 13.6 | 0.01 | 5.6 | Translation initiation | Rattus norvegicus |
| | | | | 4B | | | | | |
| 434 | −1.51 | 0.0057 | gi\|42542422 | Heat shock protein 8 | 21.5 | 0 | 5.3 | | Mus musculus |
| 724 | 2.38 | 8.40E−12 | gi\|13097417 | FK506 binding protein 4 | 25.8 | 0.002 | 5.6 | | Mus musculus |
| 1171 | 1.51 | 0.0011 | gi\|56206424 | nucleophosmin 1 | 20.6 | 0.001 | 4.5 | | Mus musculus |
| 1330 | −2.02 | 5.60E−07 | gi\|14249130\| | LIM and SH3 protein 1 (lasp-1) | 39.9 | 0 | 6.6 | | Rattus norvegicus |
| 1358 | −1.52 | 1.30E−09 | gi\|7710036 | heterogeneous nuclear | 21.3 | 0 | 6.9 | | Mus musculus |
| | | | | ribonucleoprotein D-like | | | | | |
| 1459 | −1.51 | 8.10E−09 | gi\|59858367 | annexin 5 | 20.6 | 0.001 | 4.9 | | Bos taurus |
| 1486 | 1.7 | 2.80E−10 | gi\|77377292 | sulfatase modifying factor 2 | 23.4 | 0.006 | 6.6 | | Mus musculus |
| 1532 | −2.19 | 6.70E−09 | gi\|73961099 | PREDICTED: similar to tropomyosin 3 | 29.4 | 0.001 | 4.7 | Intermediate filament | Canis familiaris |
| | | | | isoform 2 isoform 17 | | | | | |
| 1629 | −1.54 | 1.20E−10 | gi\|62647453 | PREDICTED: similar to ribose 5- | 21.5 | 0.001 | 7.9 | | Rattus norvegicus |
| | | | | phosphate isomerase | | | | | |
| 1780 | −1.6 | 9.30E−10 | gi\|32452351 | CAP1 protein | 32.3 | 0.007 | 6.3 | | Mesocricetus |
| | | | | | | | | | auratus |
| 1906 | −1.51 | 3.30E−07 | gi\|14625464 | stathmin | 53.7 | 0 | 5.9 | | Mus musculus |
| 2098 | −2.01 | 6.00E−14 | gi\|51854249 | S100 calcium binding protein A11 | 21.4 | 0 | 5.6 | | Rattus norvegicus |
| | | | | (calizzarin) | | | | | |
| 2130 | −2.61 | 4.20E−12 | gi\|198561 | EGF-binding protein | 19.6 | 0 | 6.9 | | Mus musculus |

TABLE 3
High Max Qp Prot Known Hamster Proteins

| | Average | | | | % | | | | |
|---|---|---|---|---|---|---|---|---|---|
| | Ratio | | | | cov- | | | | |
| Spot | (Test/ | | gi accession | | er- | | | | |
| no. | Control) | p-value | no. | Protein name | age | pI | Mr | Function | Species |
| 426 | −2 | 3.6 × 10−7 | gi\31981722 | heat shock 70 kDa protein 5 (glucose- | 32.1 | 5.1 | 72.53 | Protein complex | Cricetulus griseus |
| | | | or | regulated protein) or dnaK-type molecular | | | | assembly in ER? | |
| | | | gi\|90188 | chaperone GRP78 precursor - Chinese | | | | | |
| | | | | hamster | | | | | |
| 452 | −1.73 | 3.6 × 10−11 | gi\|3122170 | Stress-70 protein, mitochondrial precursor | 15.8 | 5.9 | 74 | Chaperone | Cricetulus griseus |
| | | | and | (75 kDa glucose regulated protein) (GRP | | | | | |
| | | | gi\|2231704 | 75)/70 kDa heat shock protein precursor | | | | | |
| 719 | 3.54 | 7.6 × 10−14 | gi\|123332 | Hydroxymethylglutaryl-CoA synthase, | 11.5 | 5.4 | 57.93 | Cholesterol | Cricetulus griseus |
| | | | | cytoplasmic (HMG-CoA synthase) (3- | | | | biosynthesis | |
| | | | | hydroxy-3-methylglutaryl coenzyme A | | | | | |
| | | | | synthase) | | | | | |
| 729 | 2.78 | 1.6 × 10−16 | gi\|123332 | Hydroxymethylglutaryl-CoA synthase, | 15.8 | 5.4 | 57.93 | Cholesterol | Cricetulus griseus |
| | | | | cytoplasmic (HMG-CoA synthase) (3- | | | | biosynthesis | |
| | | | | hydroxy-3-methylglutaryl coenzyme A | | | | | |
| | | | | synthase) | | | | | |
| 745 | −1.59 | 3.6 × 10−8 | gi\|860908 | vimentin | 41.6 | 4.7 | 44.62 | Intermediate filament | Cricetulus griseus |
| 750 | −1.75 | 2.7 × 10−7 | gi\|860908 | vimentin | 51.6 | 4.7 | 44.62 | Intermediate filament | Cricetulus griseus |
| 778 | −1.65 | 1 × 10−9 | gi\|860908 | vimentin | 26.6 | 4.7 | 44.62 | Intermediate filament | Cricetulus griseus |
| 867 | −2.33 | 4.6 × 10−9 | gi\|860908 | vimentin | 26.6 | 4.7 | 44.62 | Intermediate filament | Cricetulus griseus |
| 947 | −2.02 | 7.2 × 10−7 | gi\|860908 | vimentin | 45.5 | 4.7 | 44.62 | Intermediate filament | Cricetulus griseus |
| 1308 | −2.38 | 2.70E−14 | gi\|2114406 | aldo-keto reductase | 23.7 | 6.2 | 36.61 | | Cricetulus gnseus |

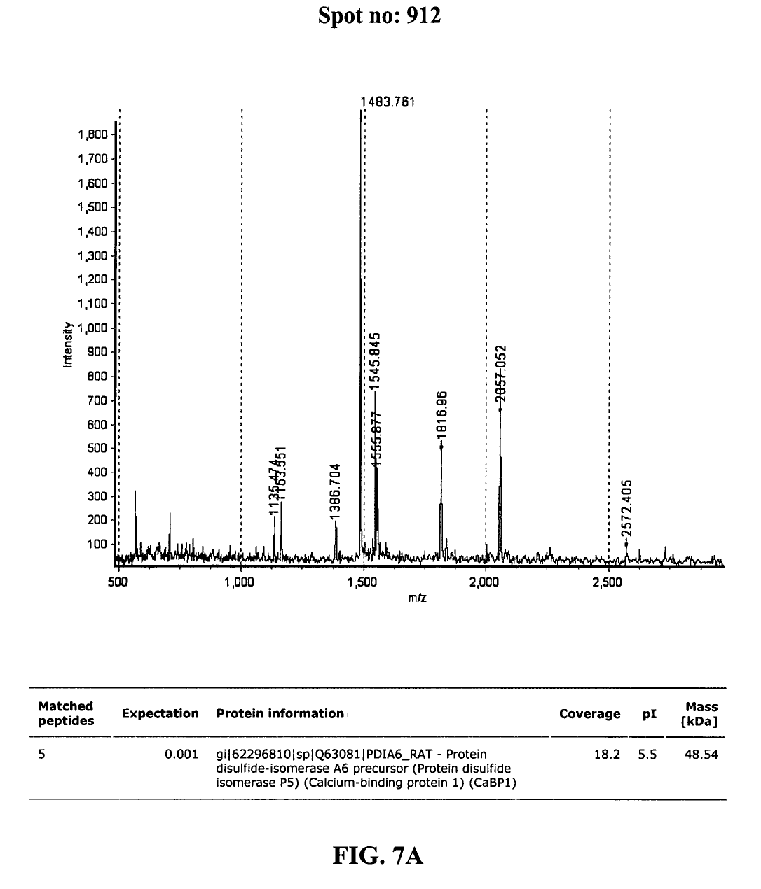

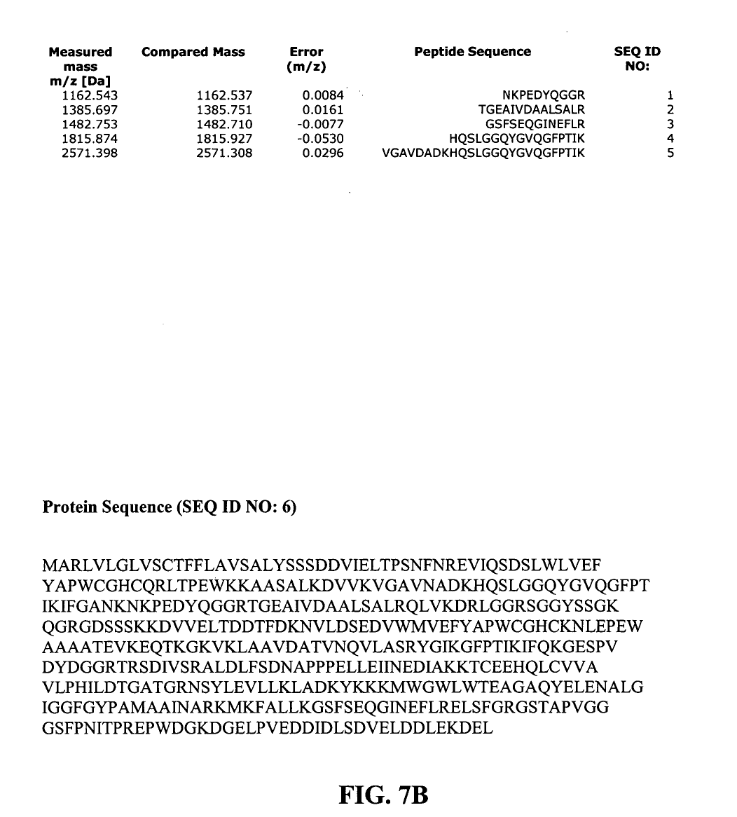

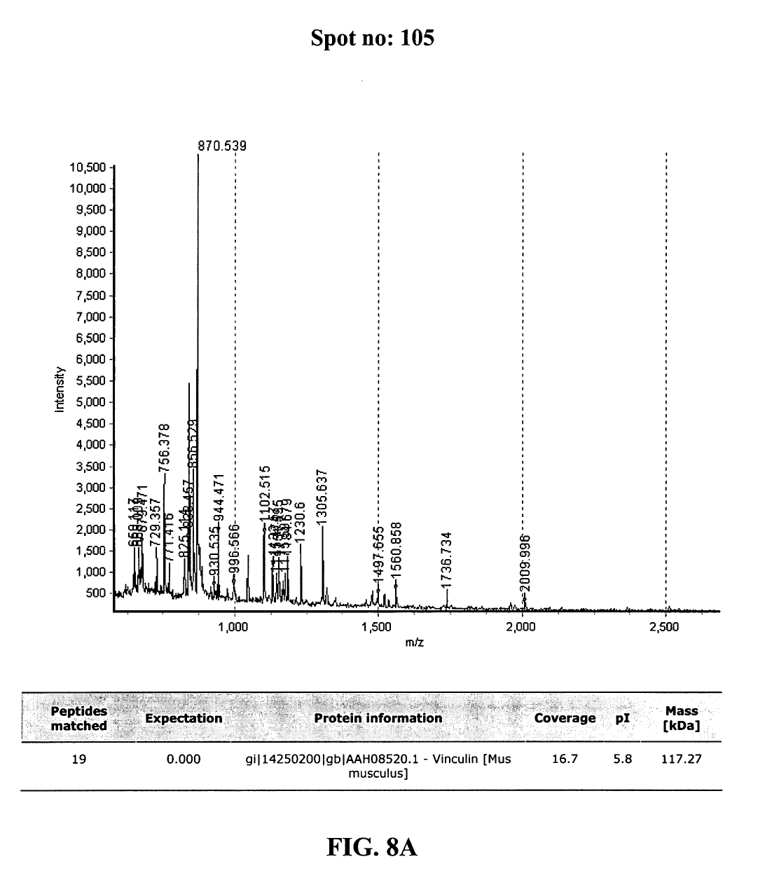

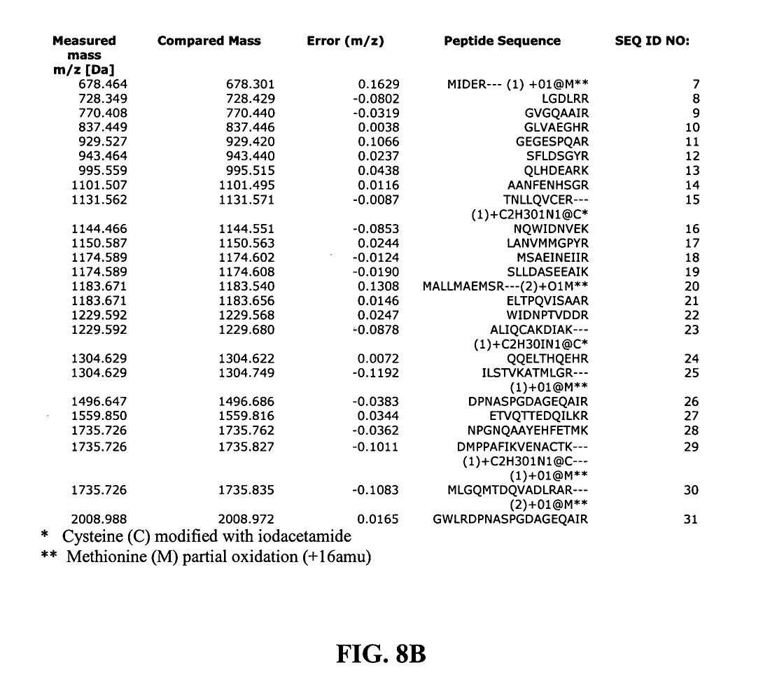

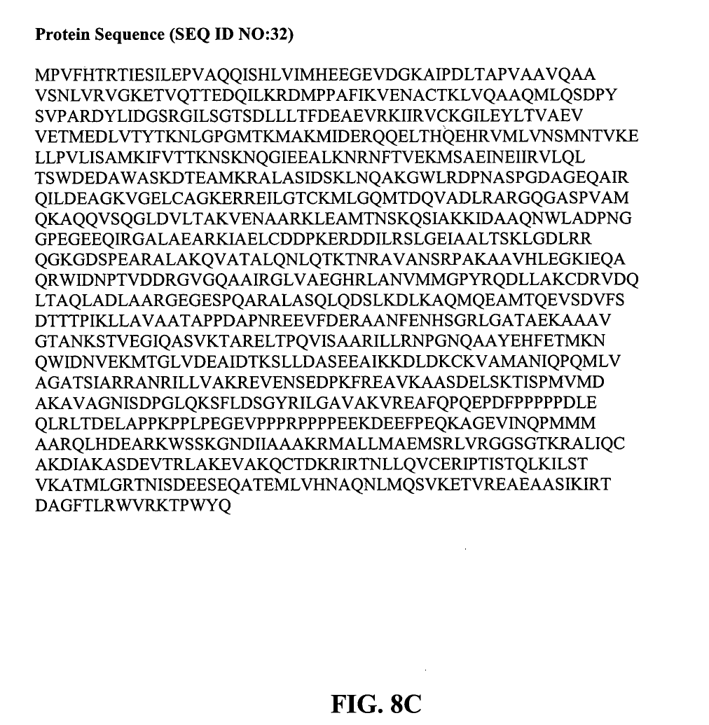

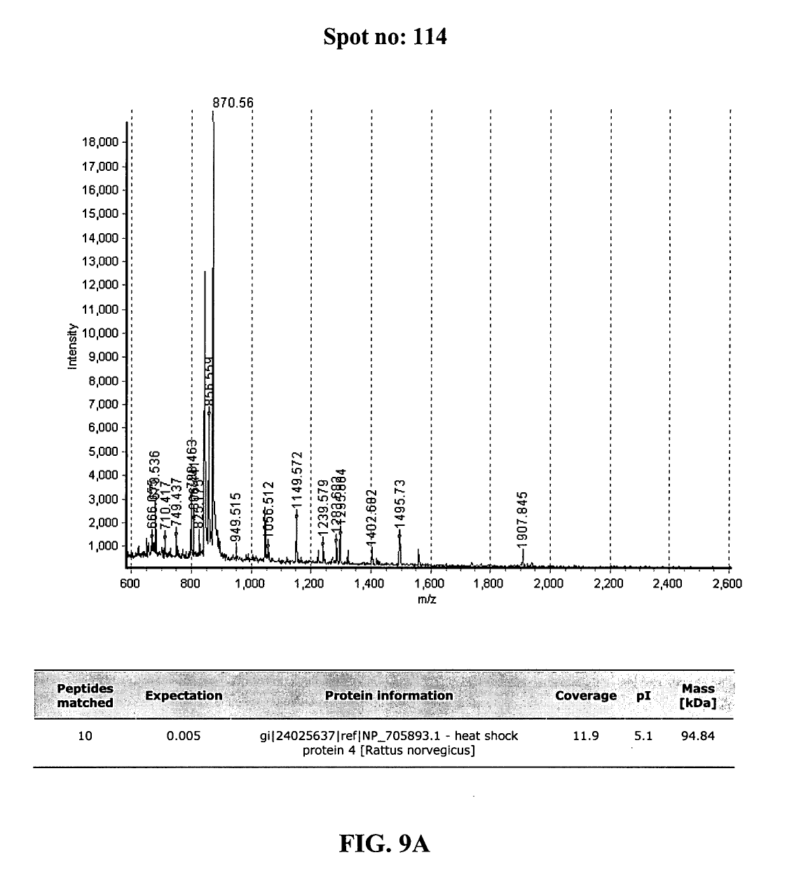

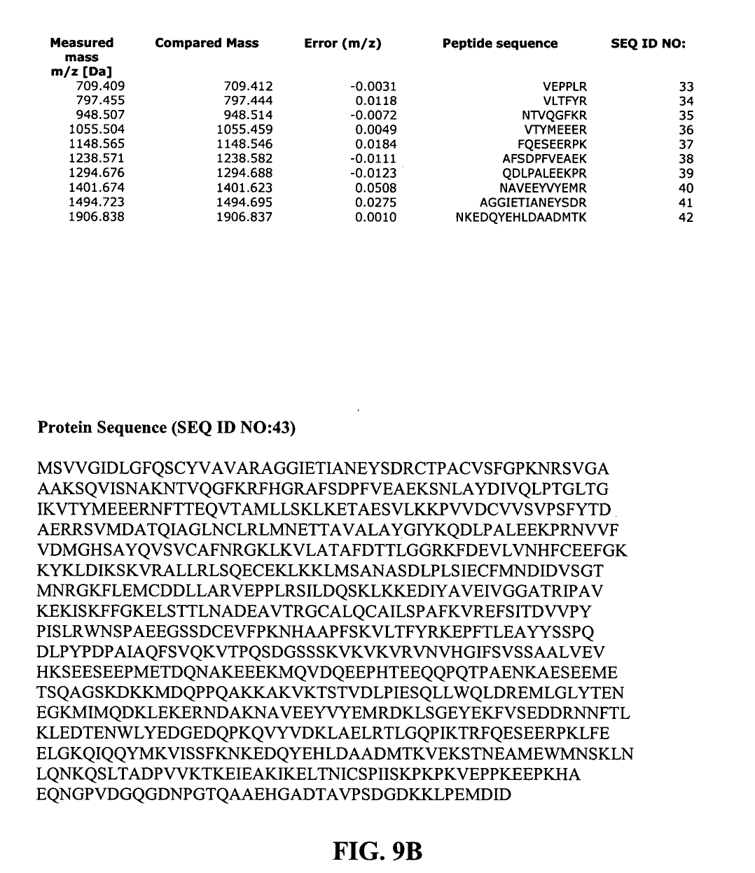

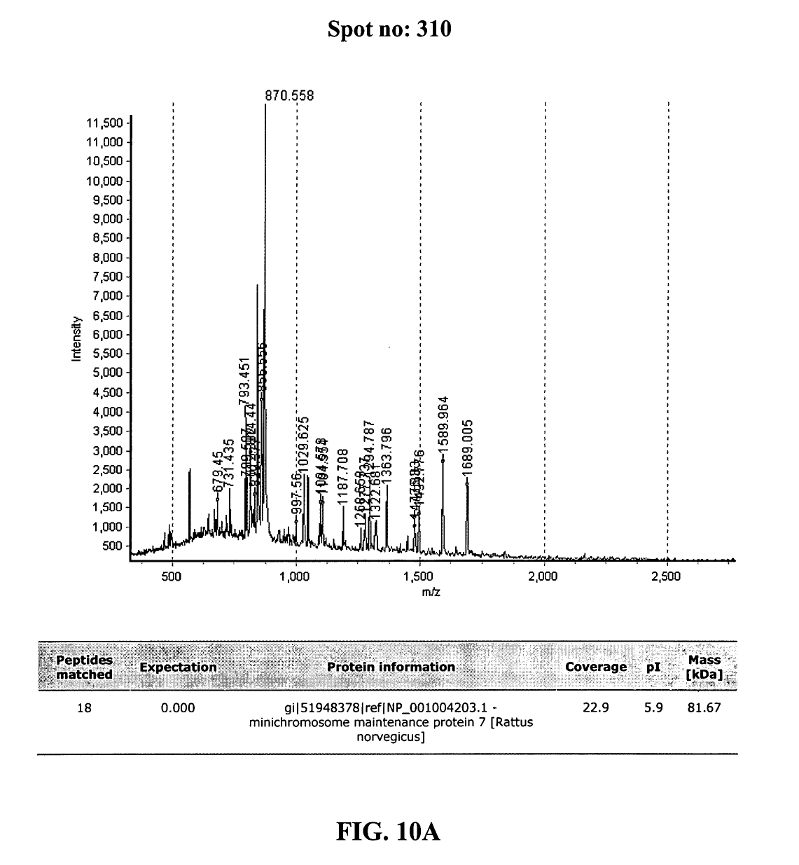

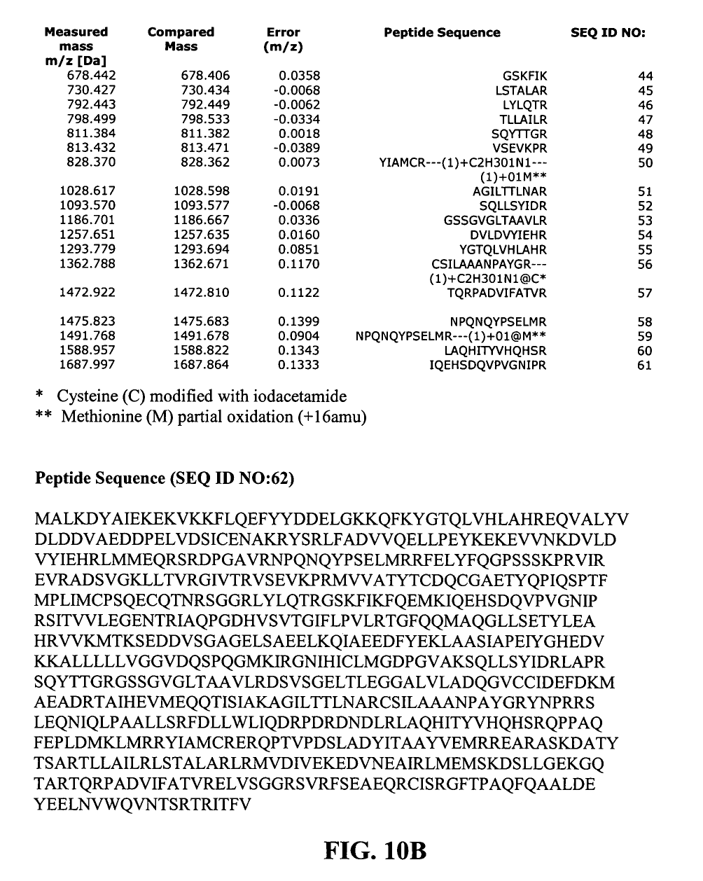

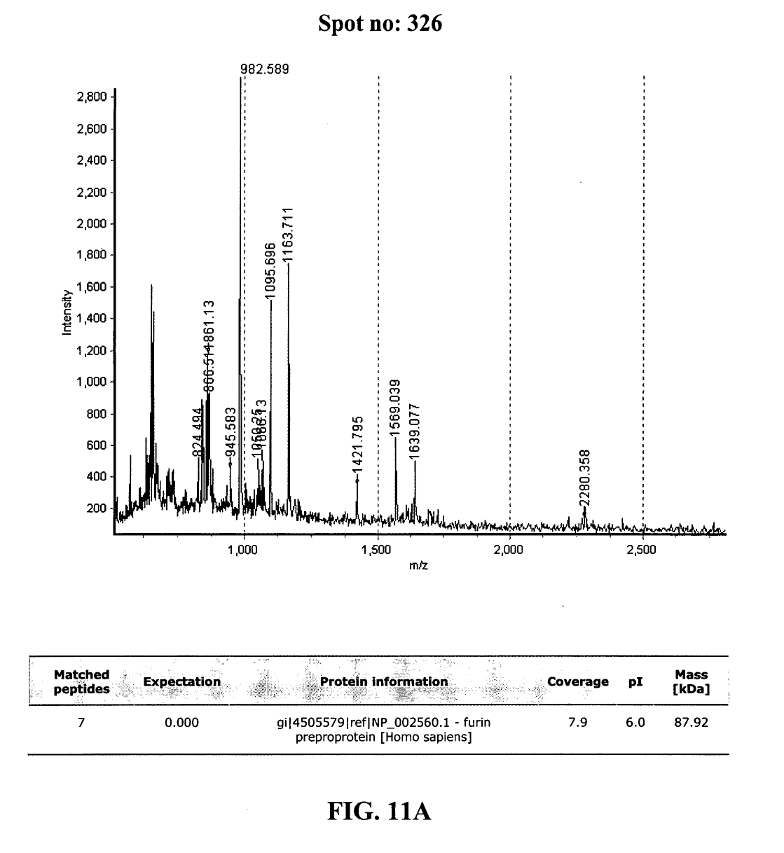

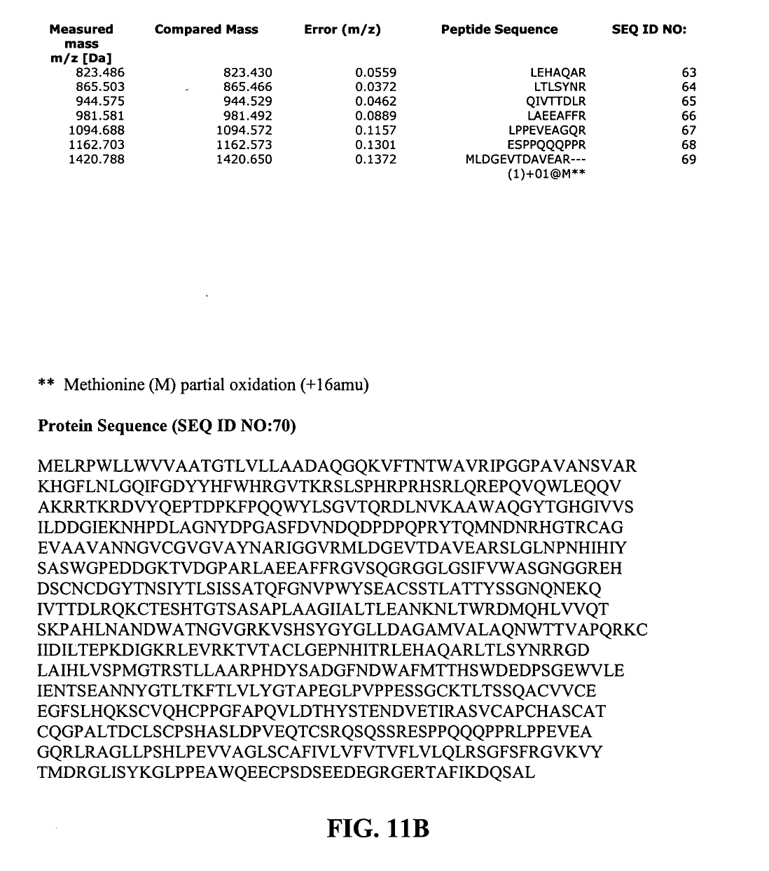

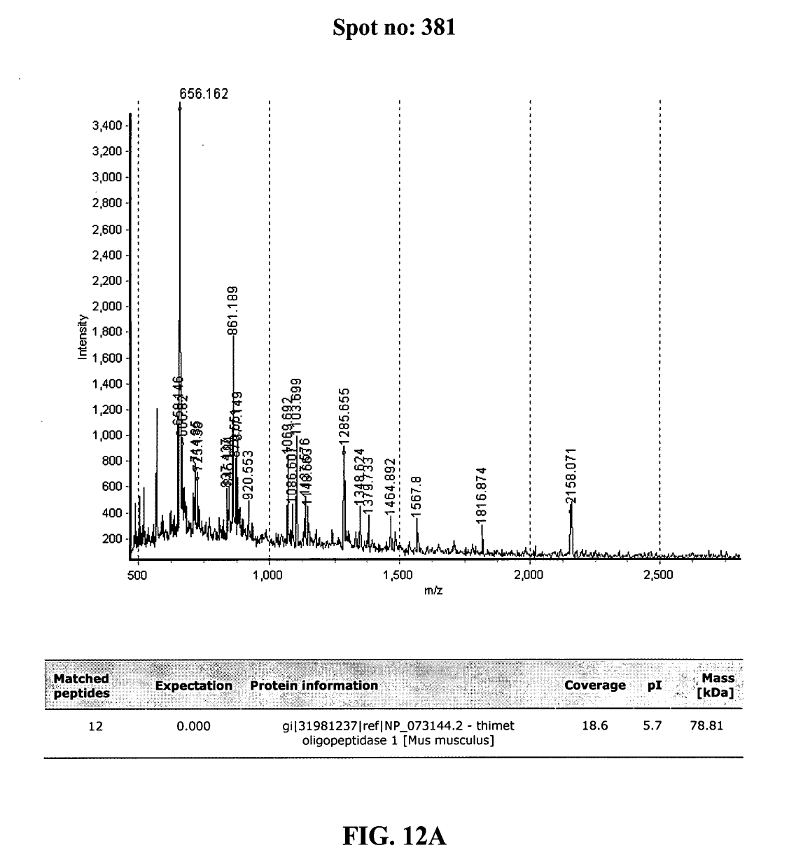

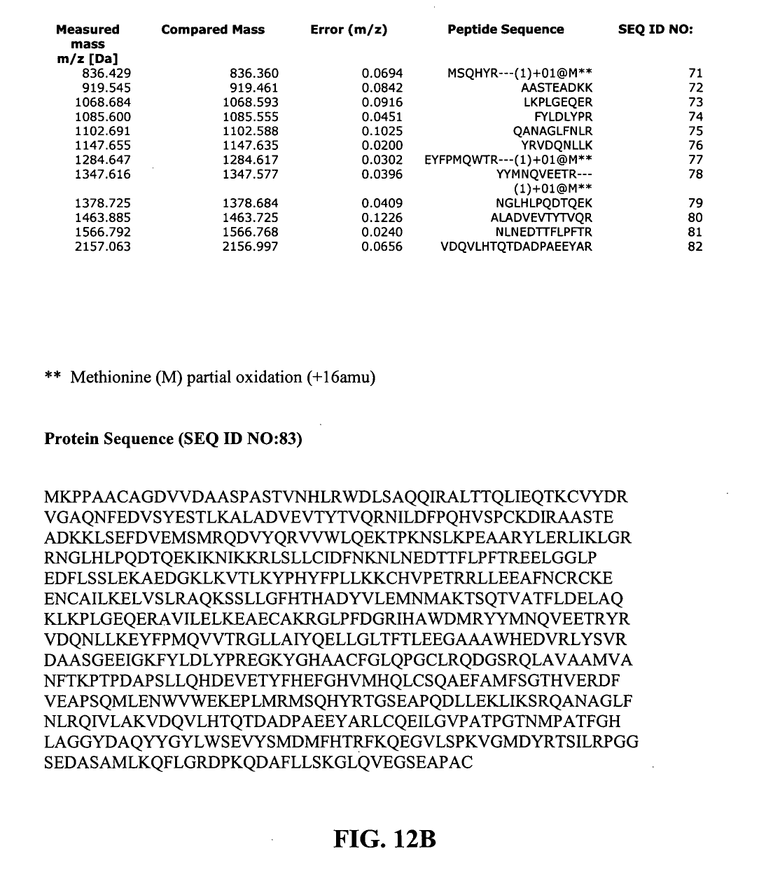

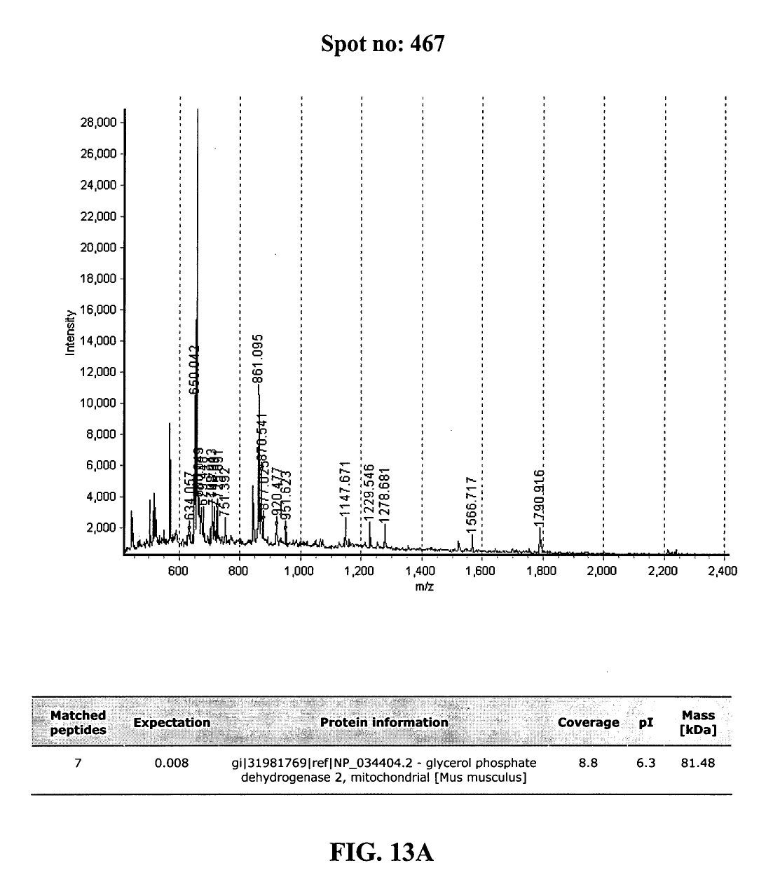

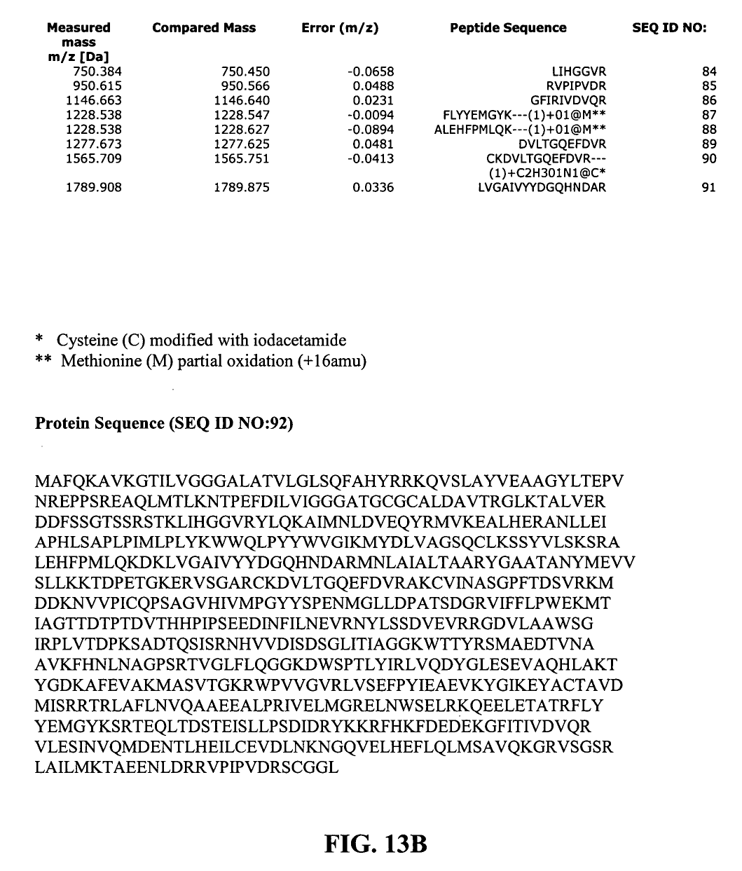

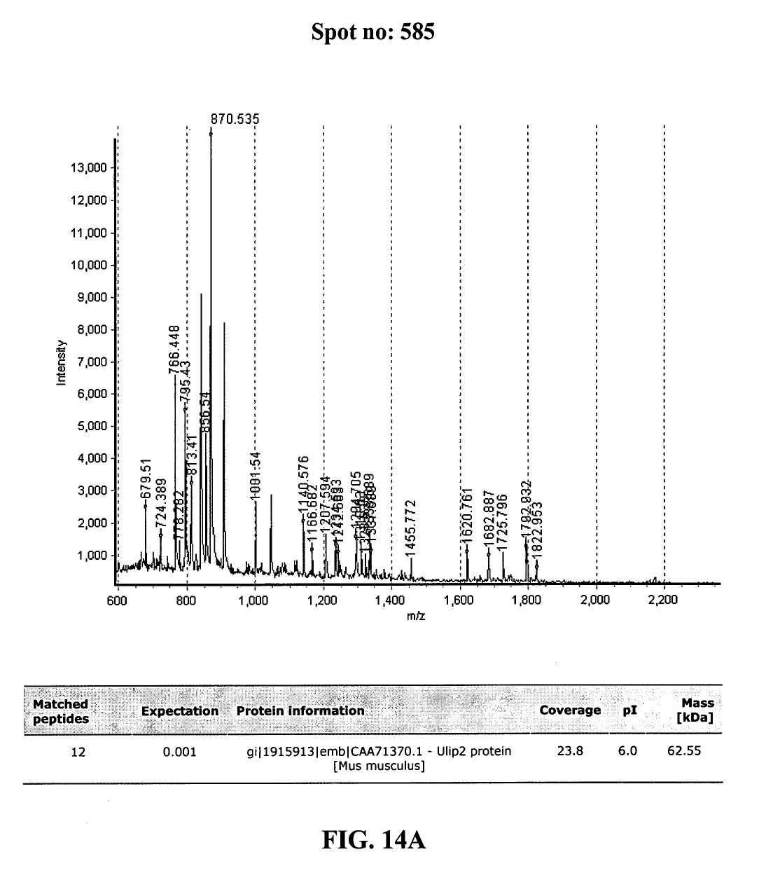

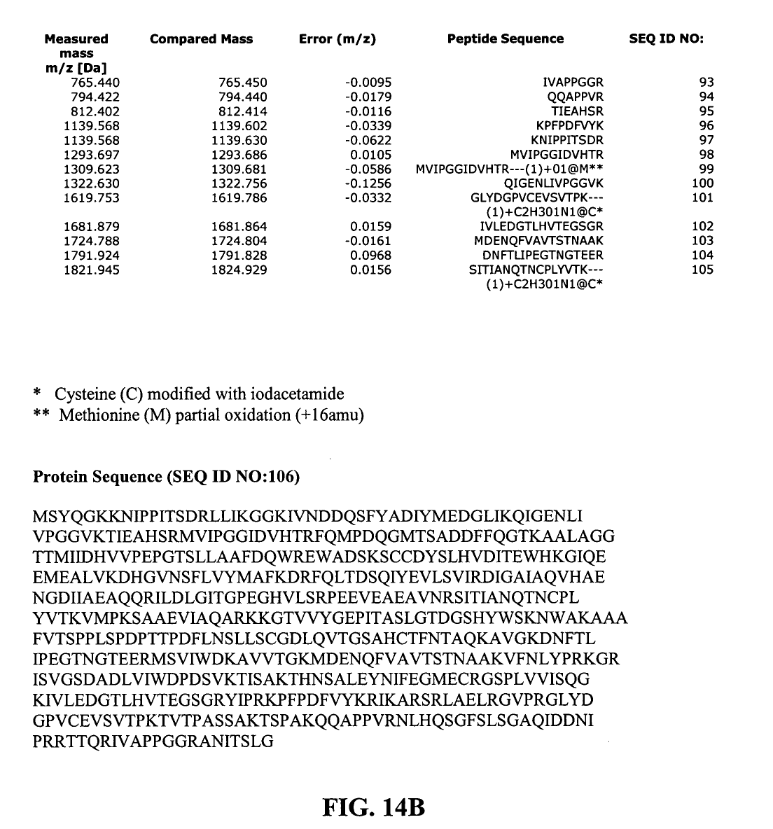

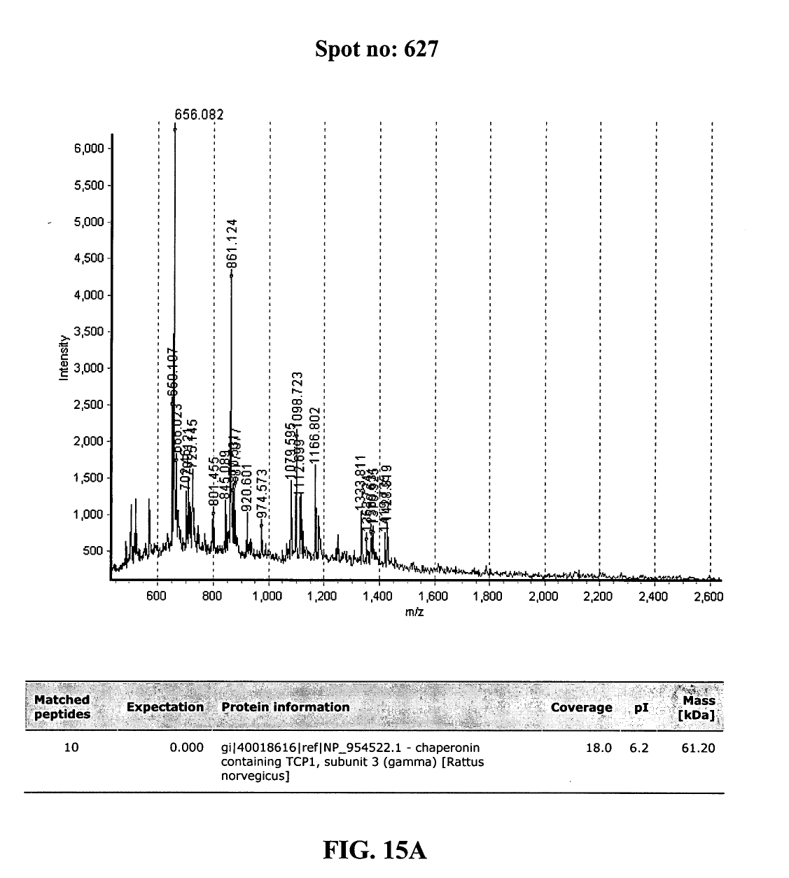

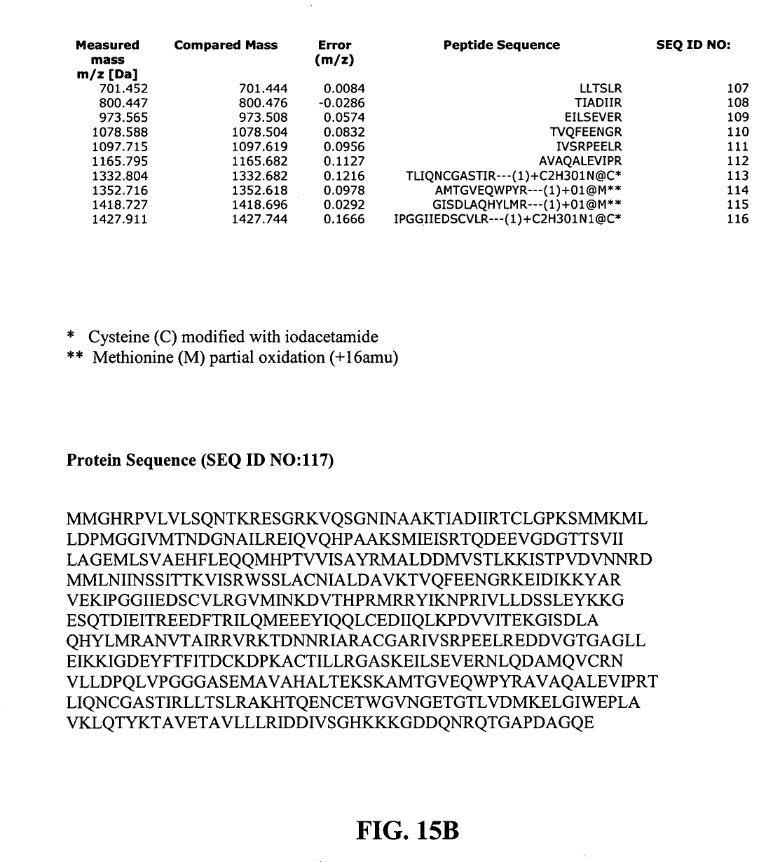

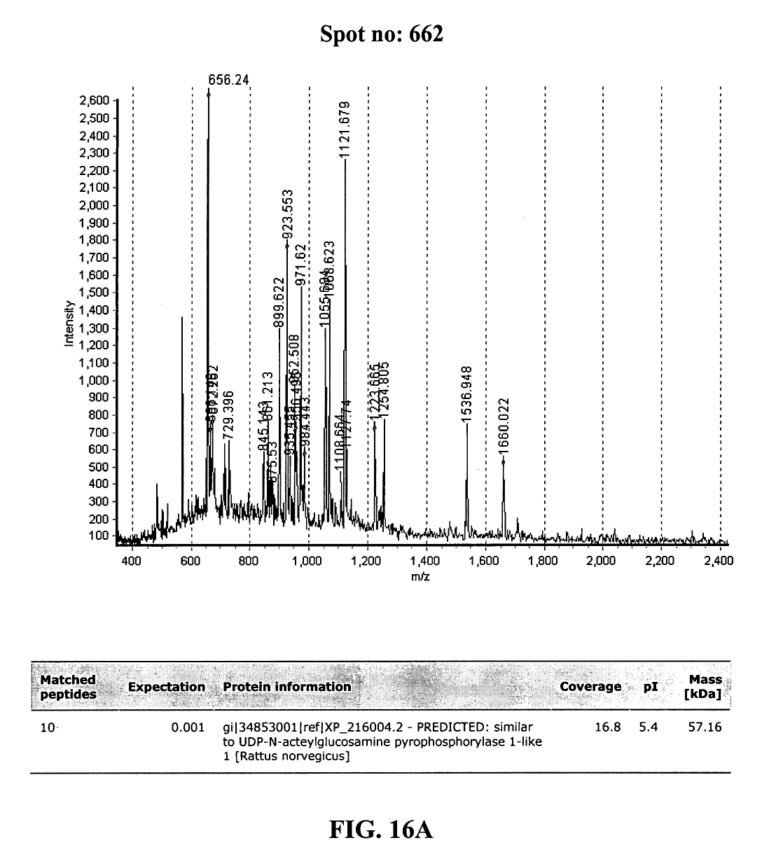

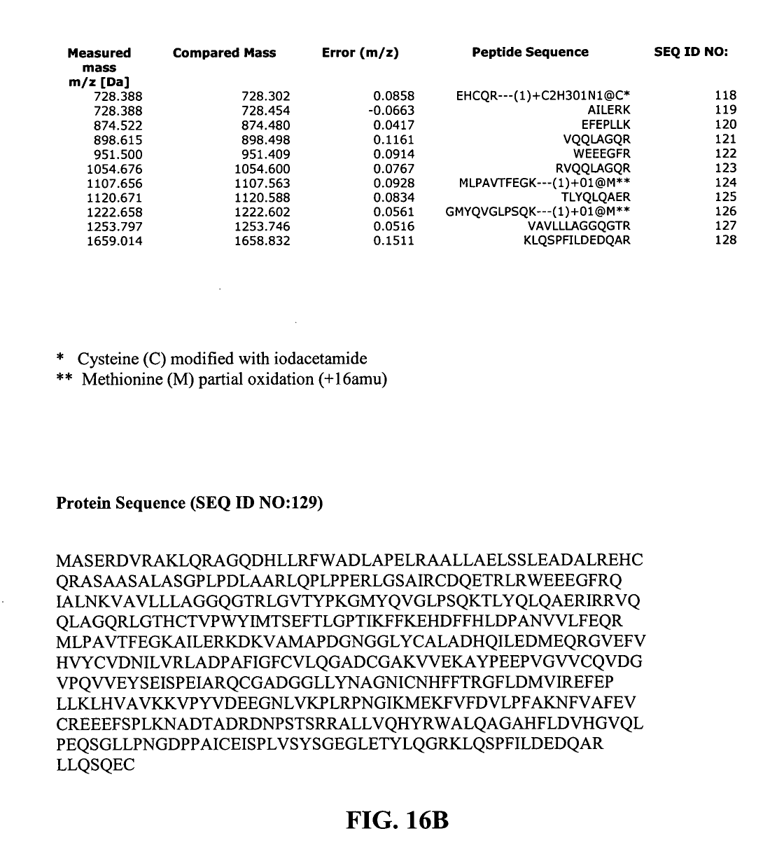

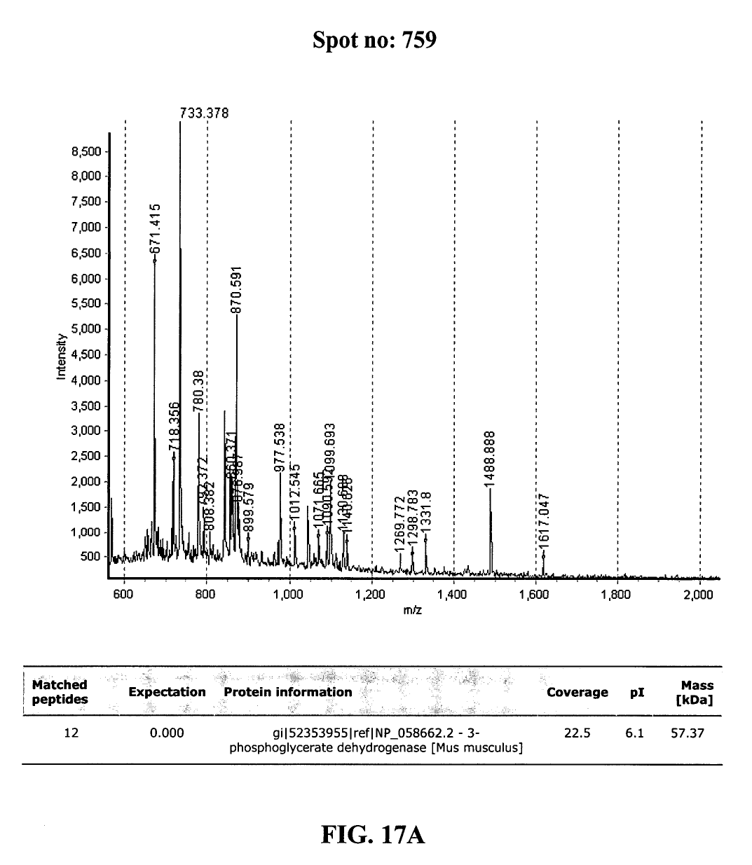

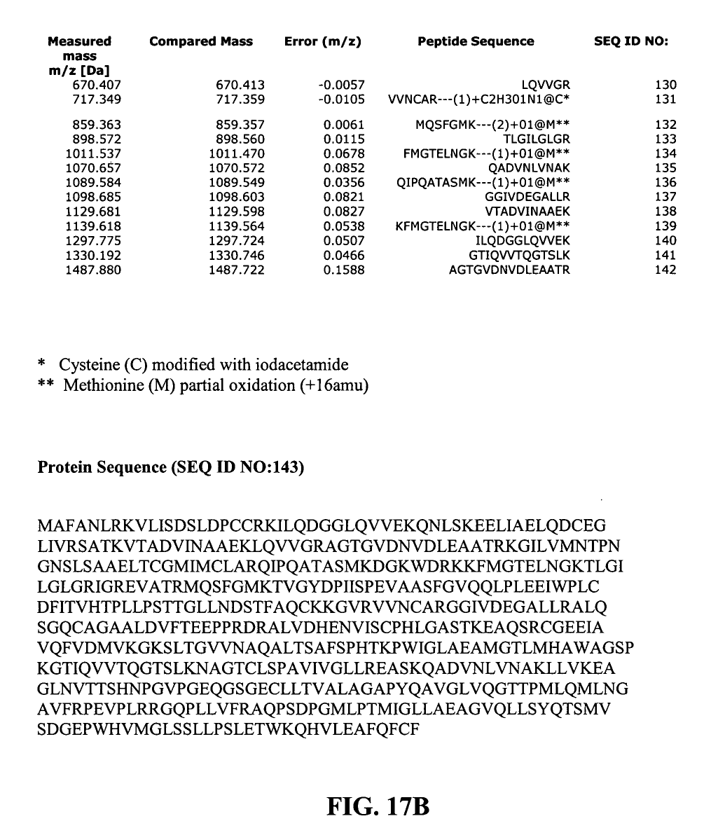

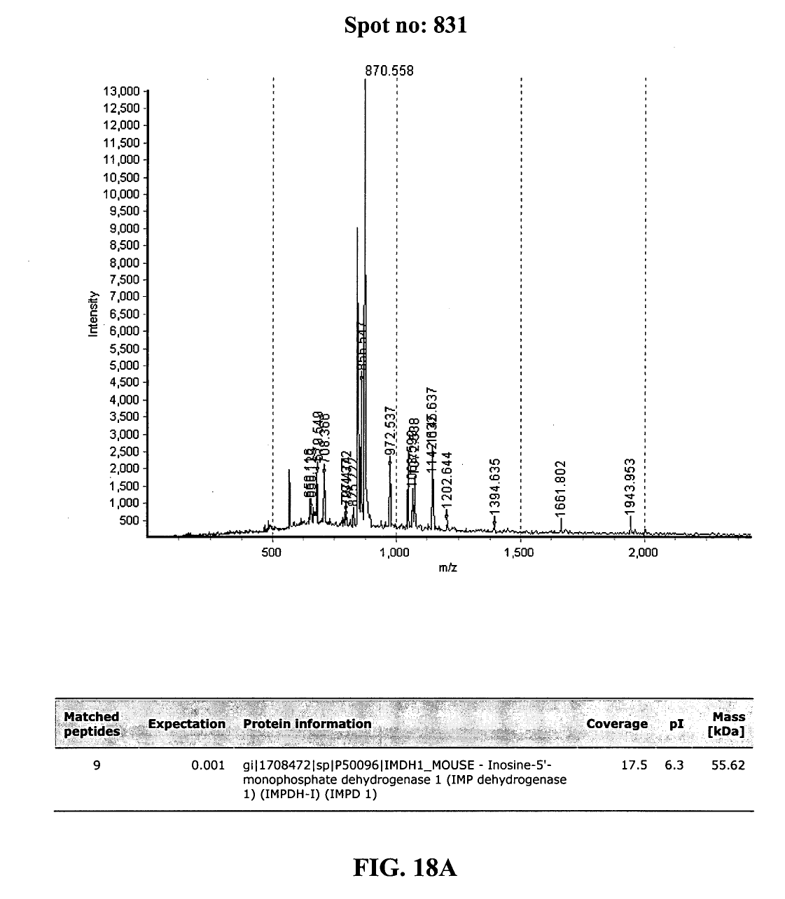

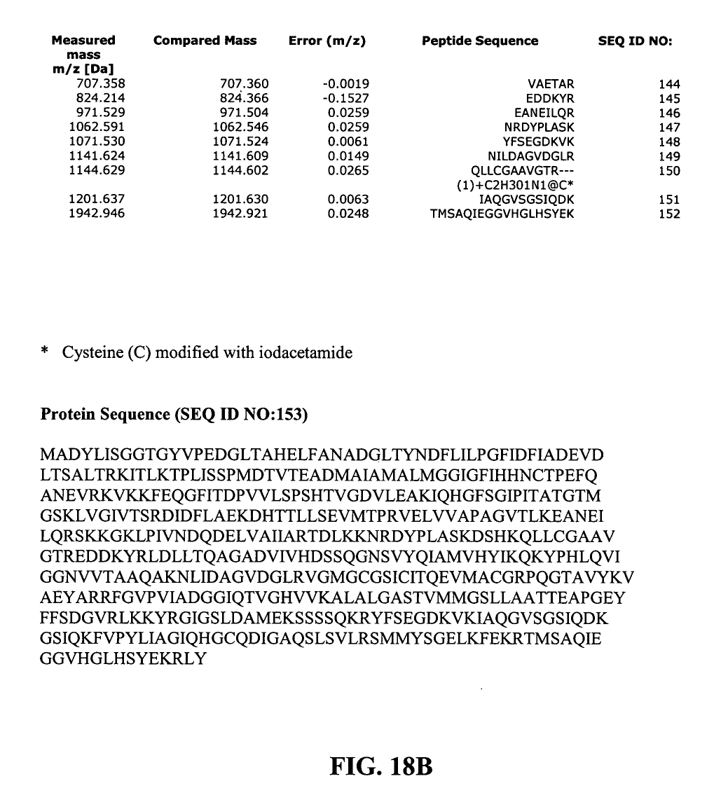

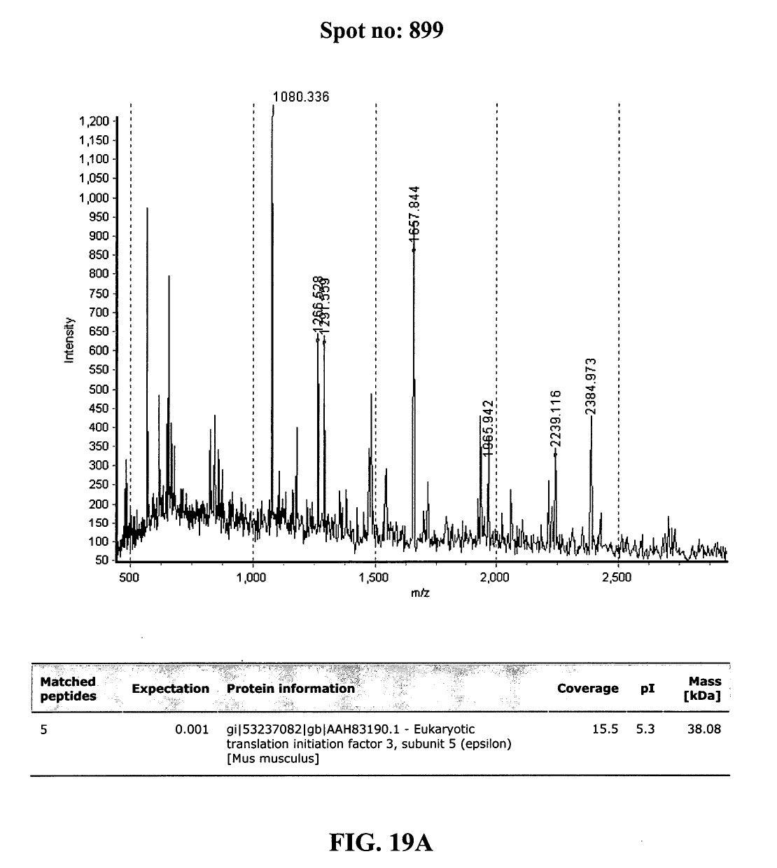

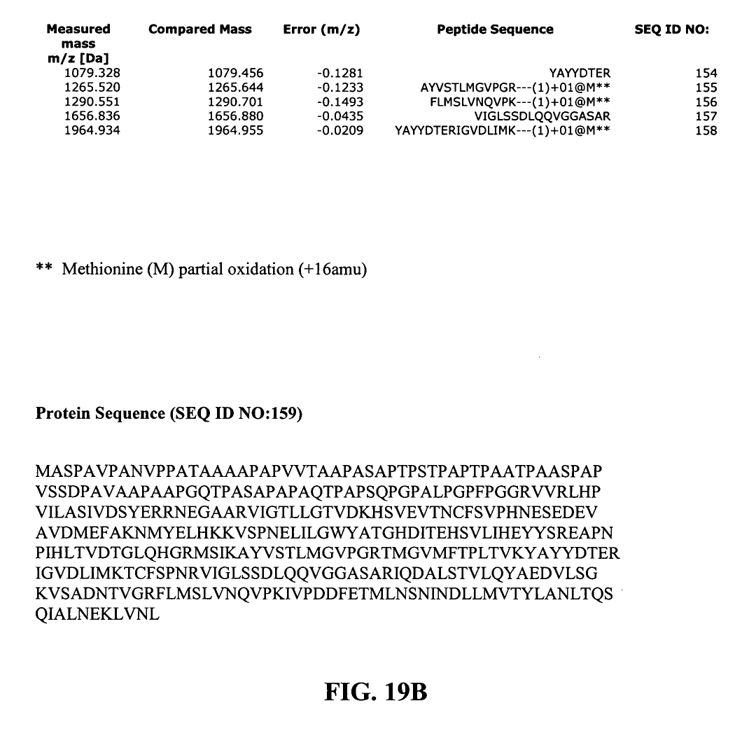

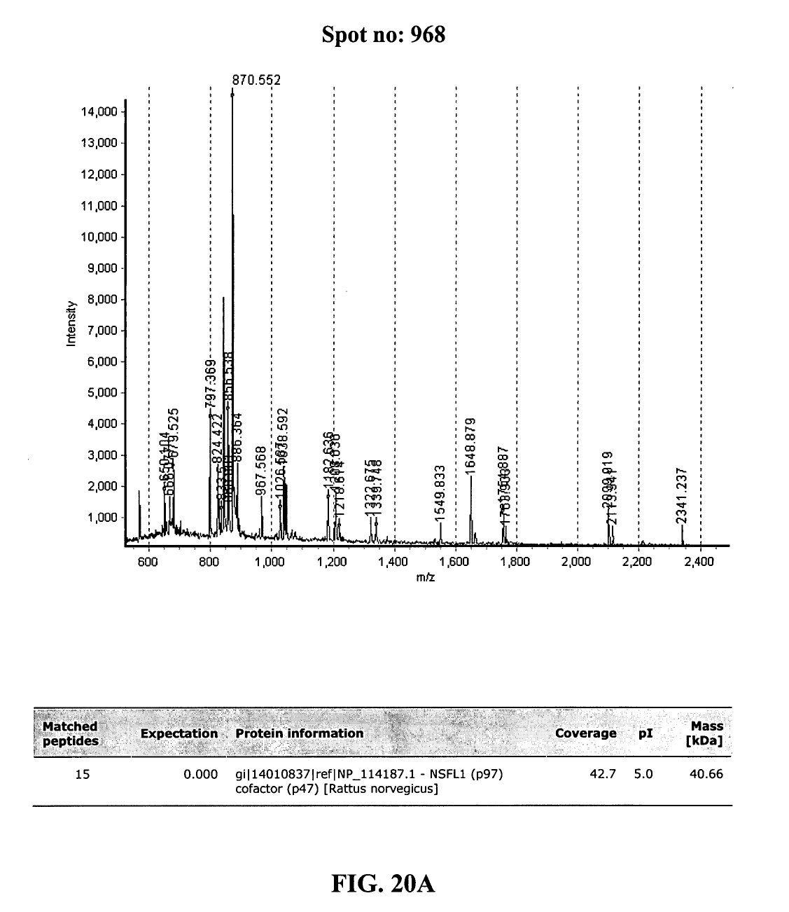

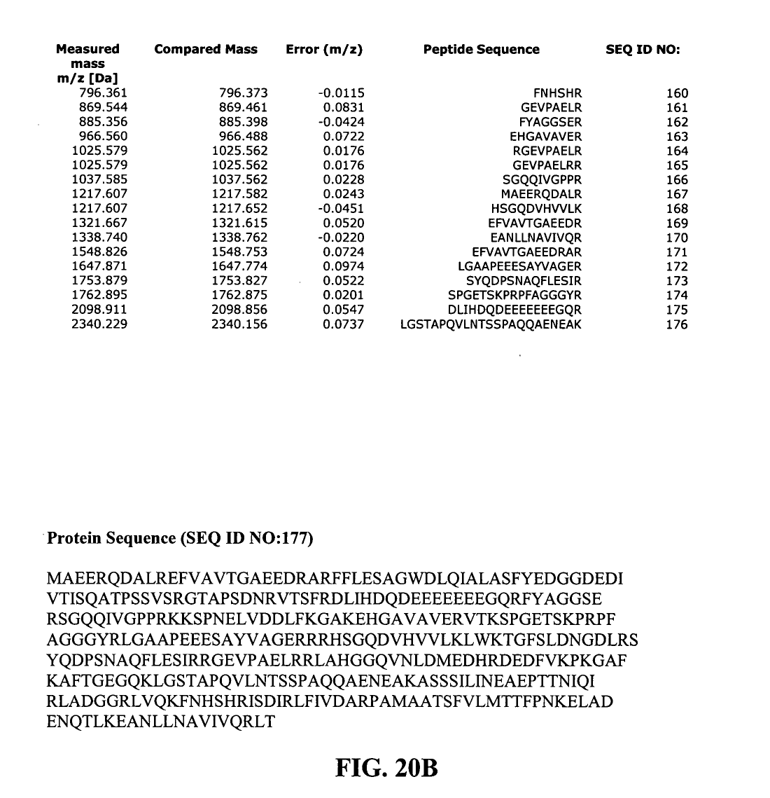

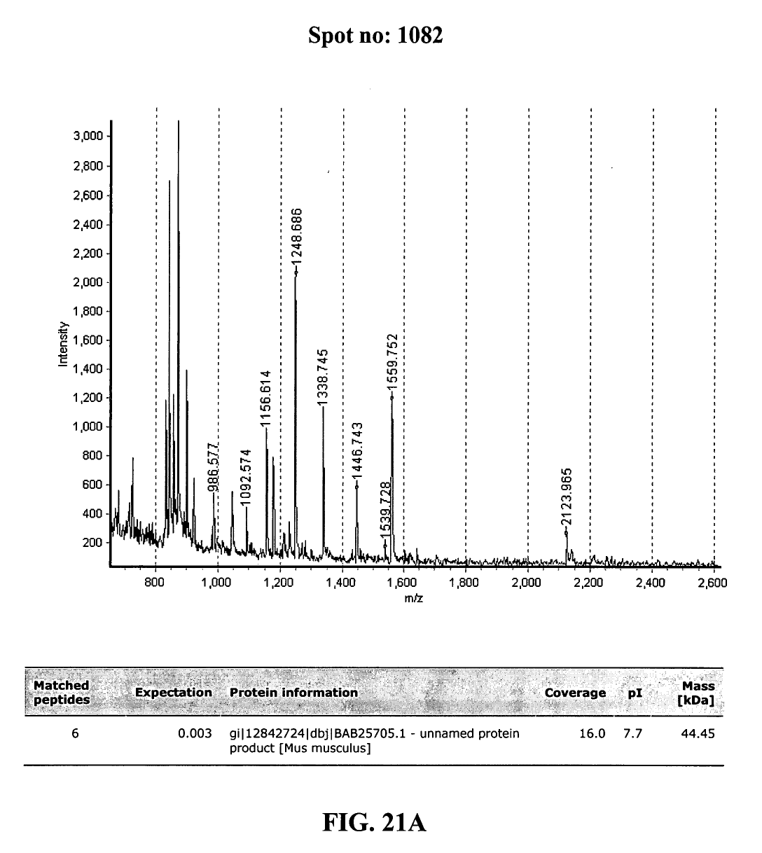

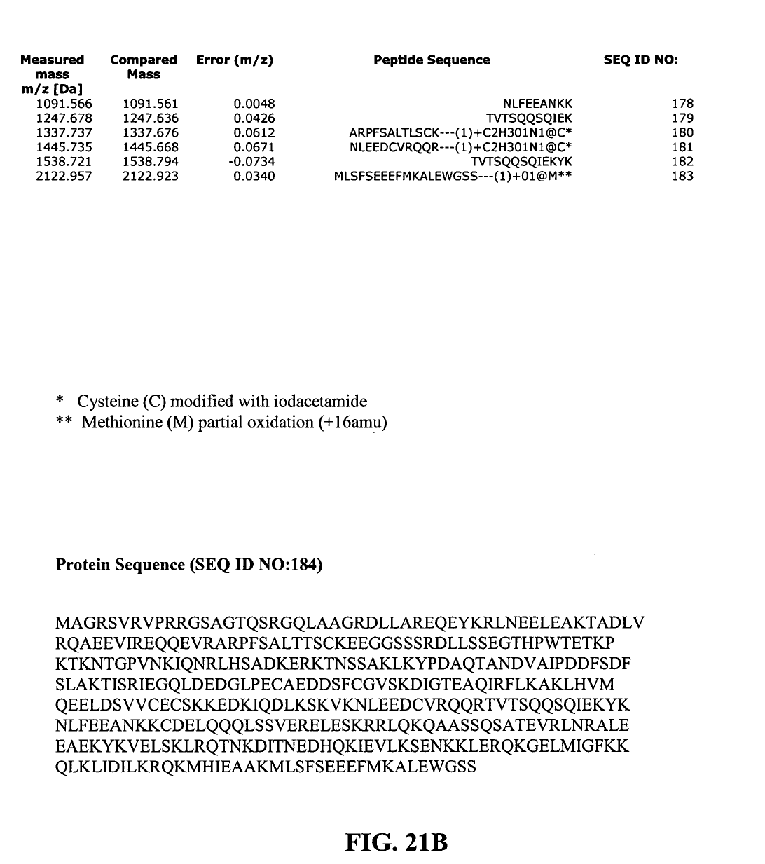

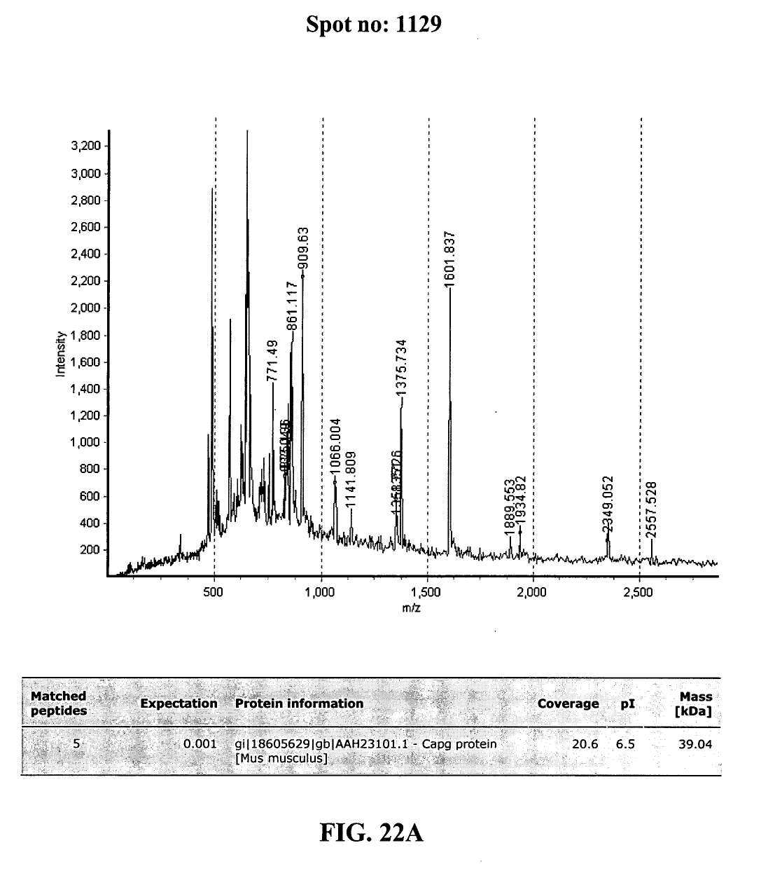

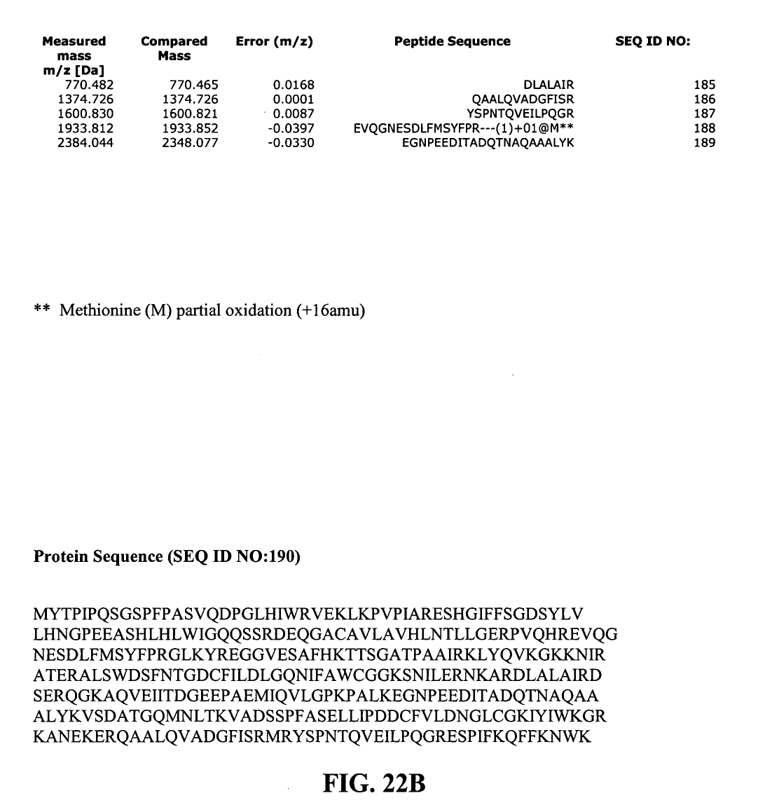

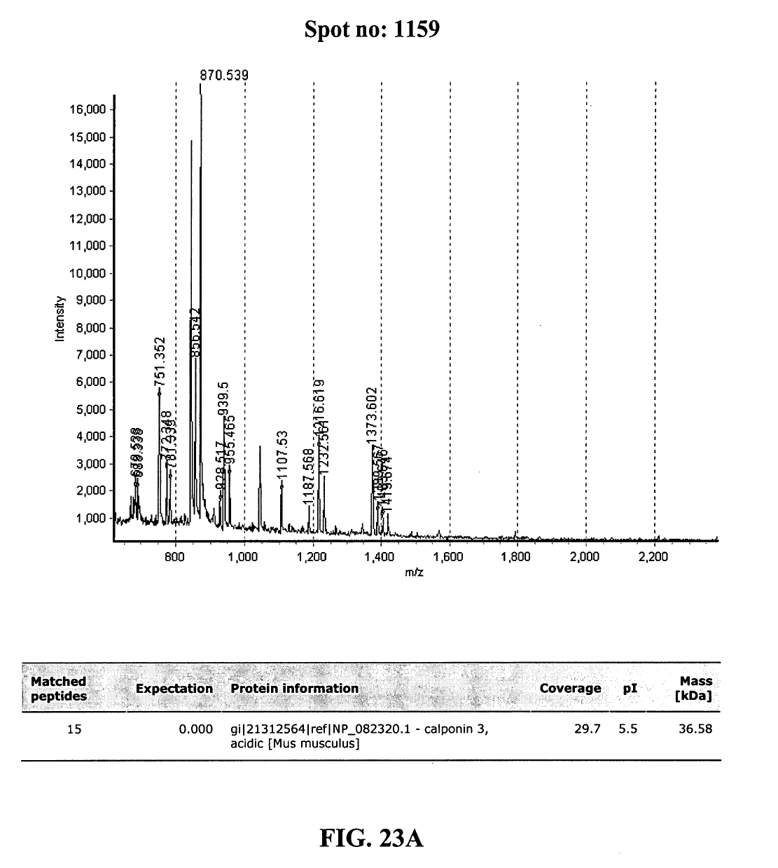

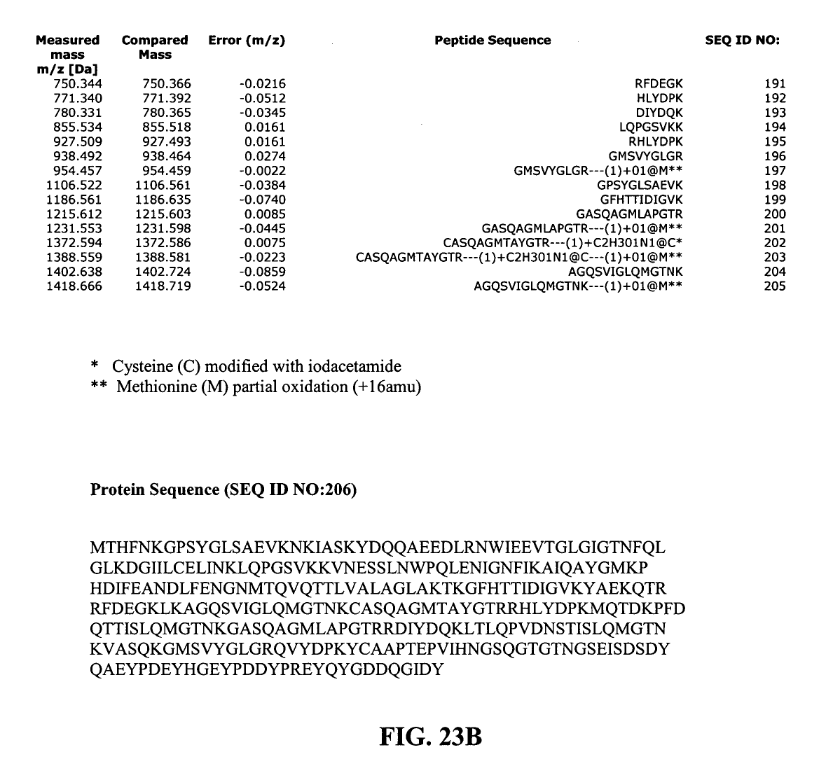

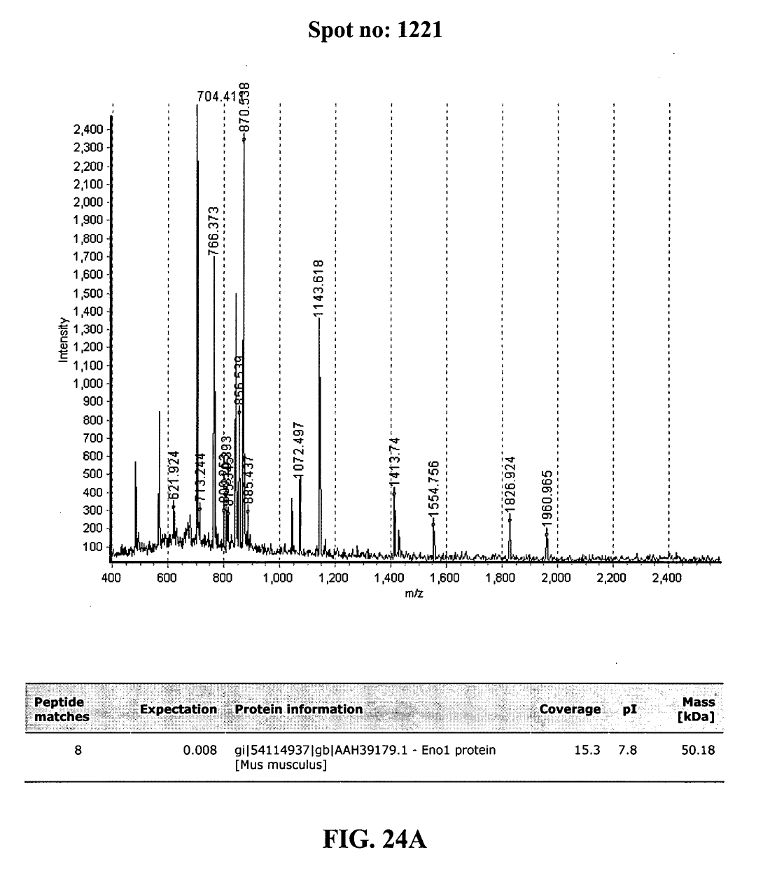

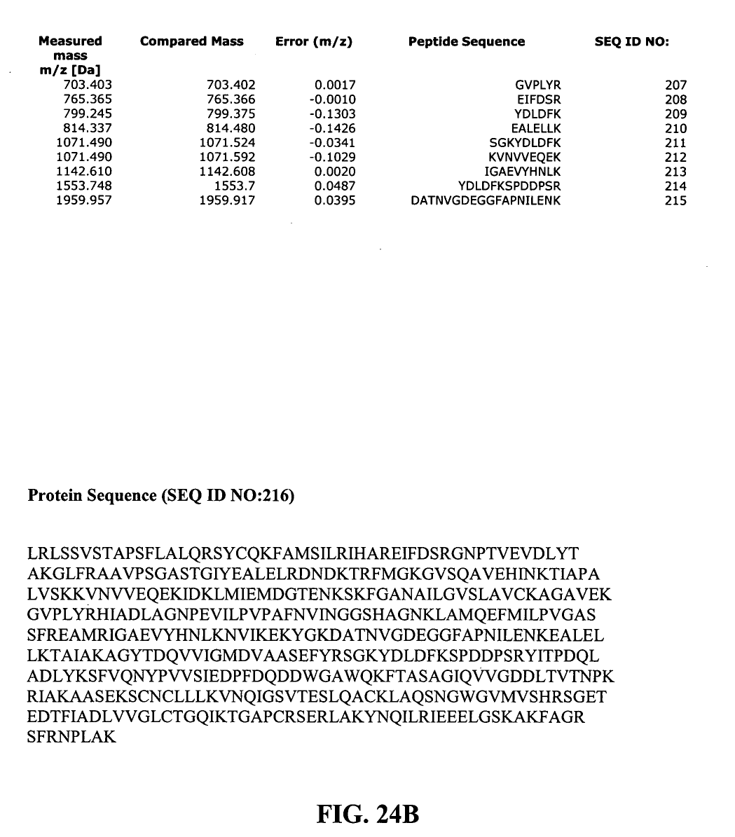

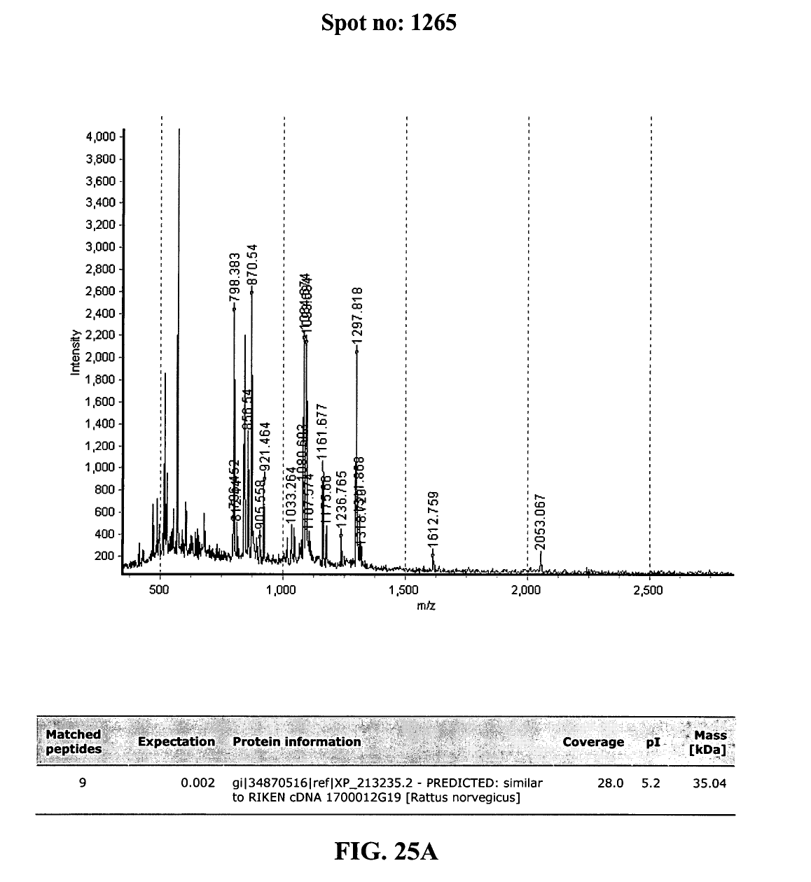

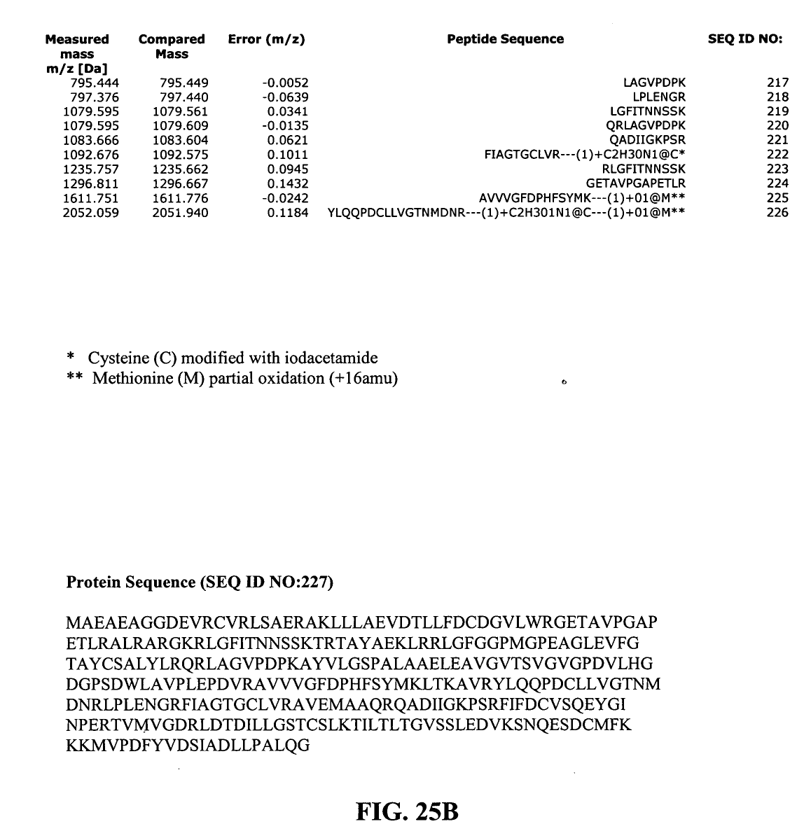

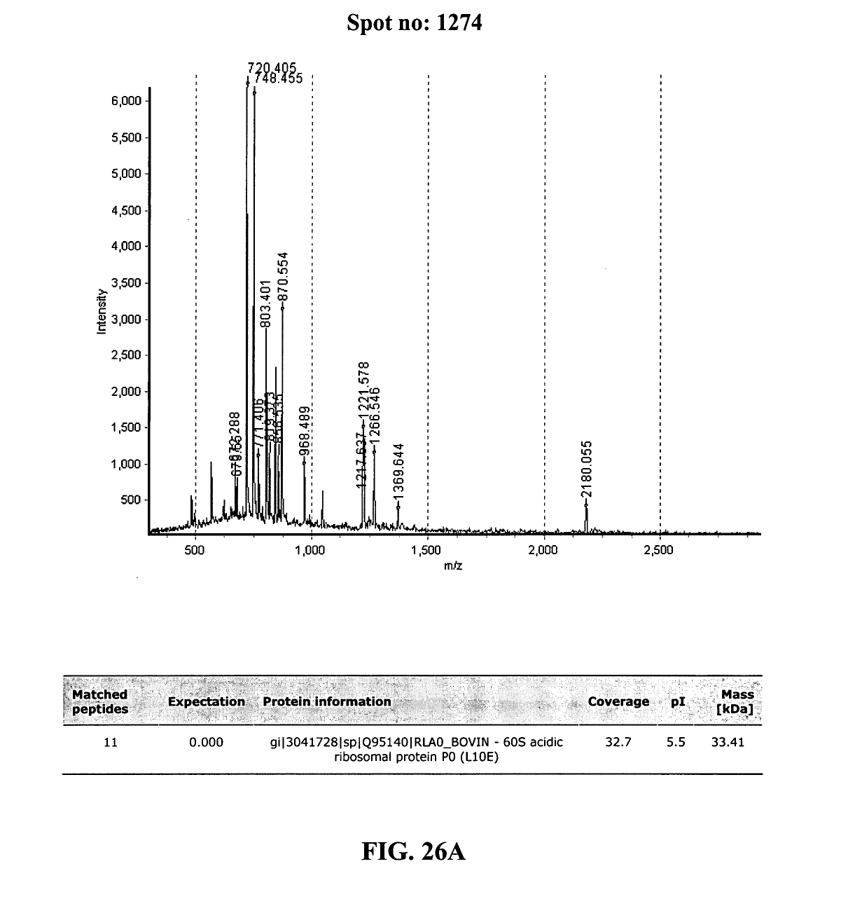

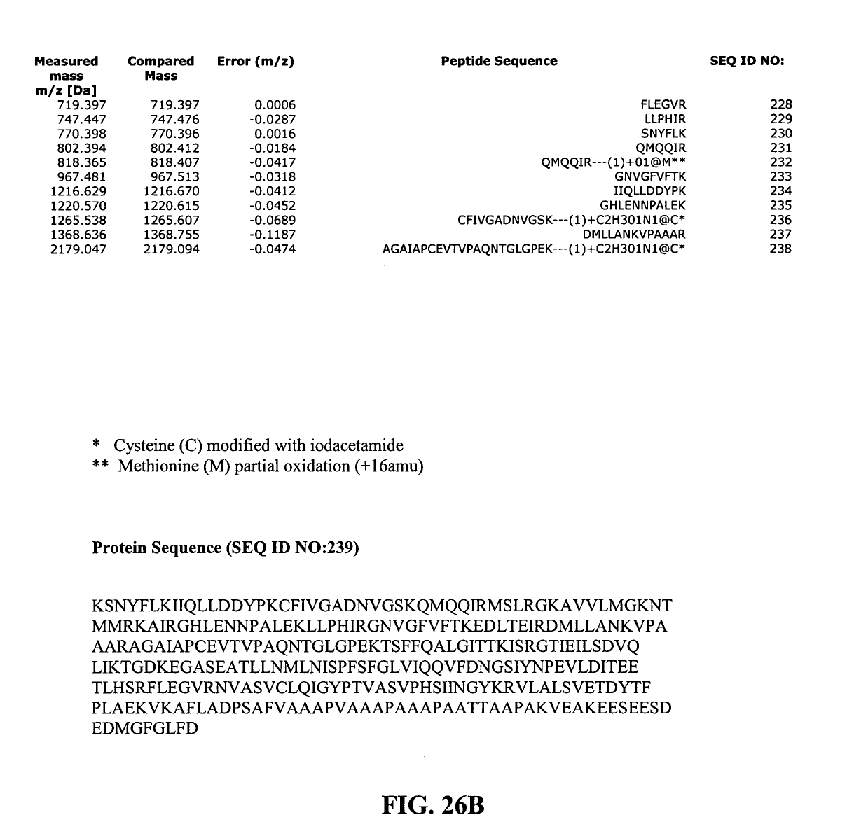

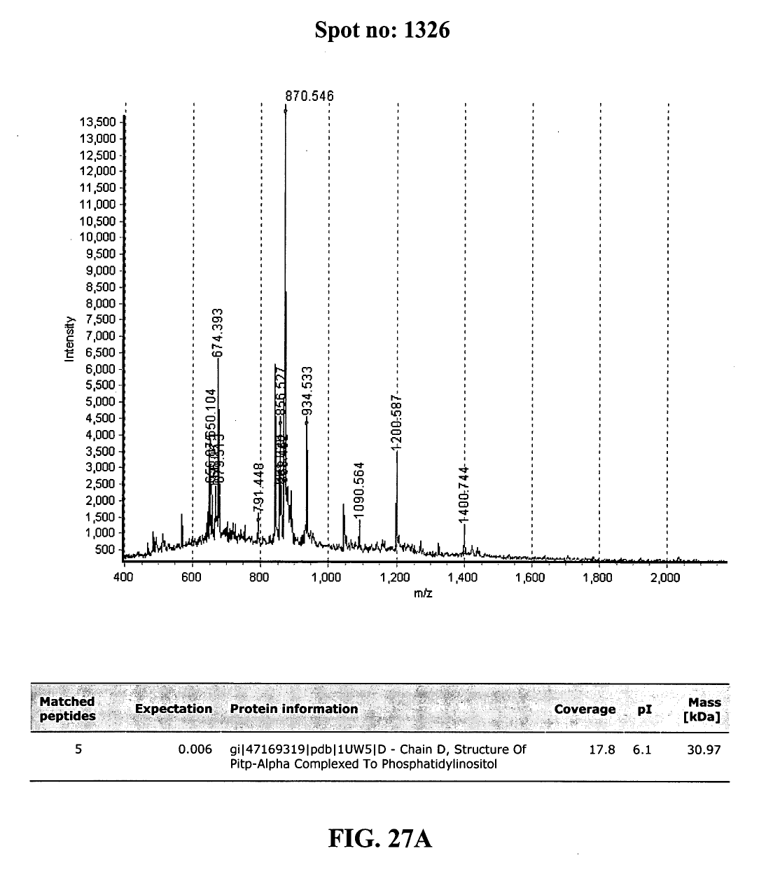

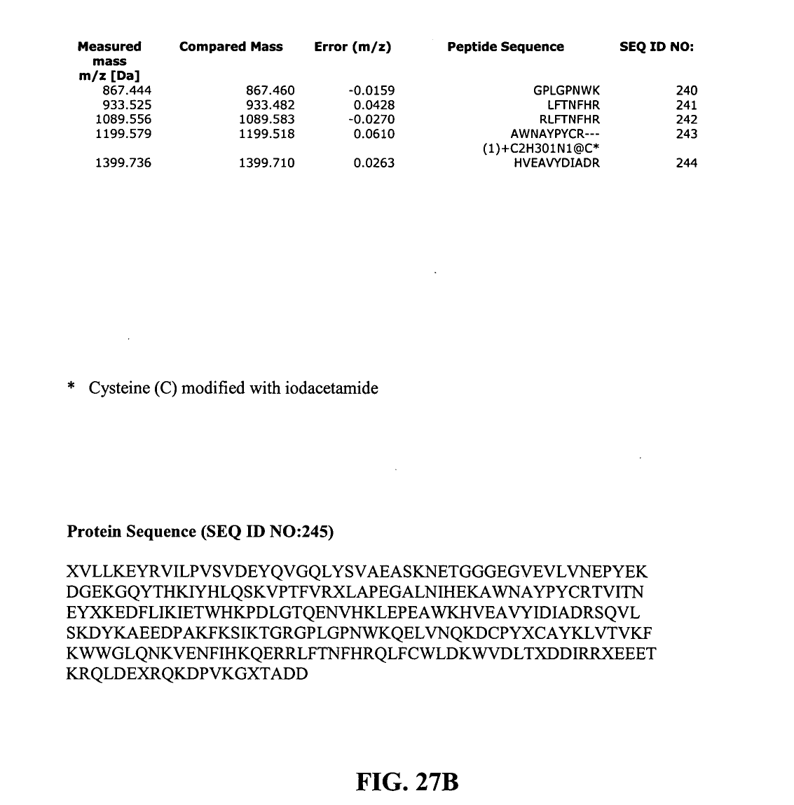

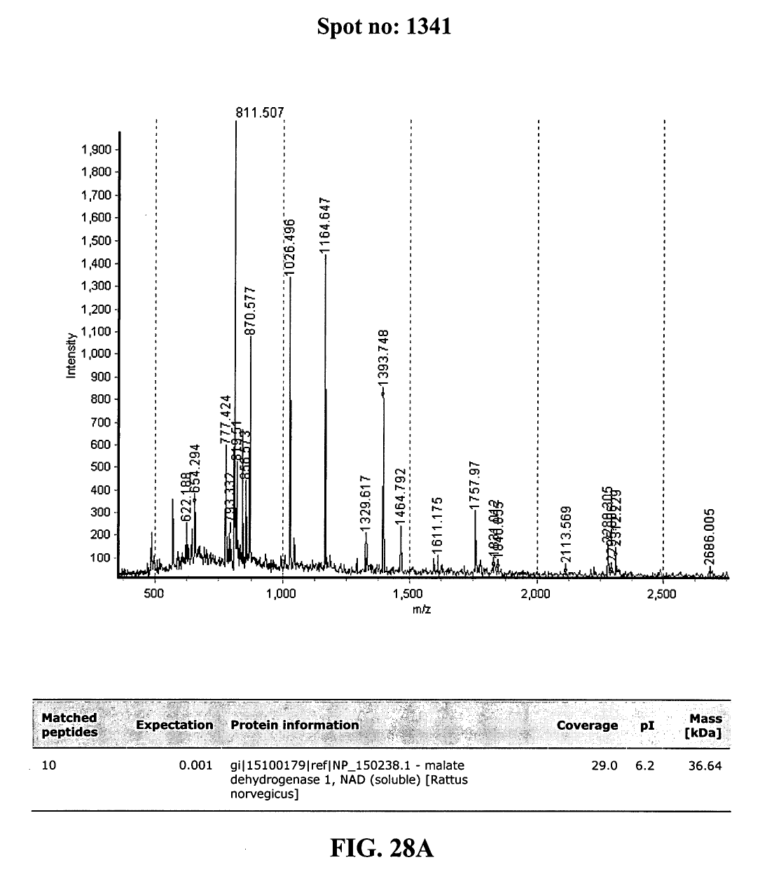

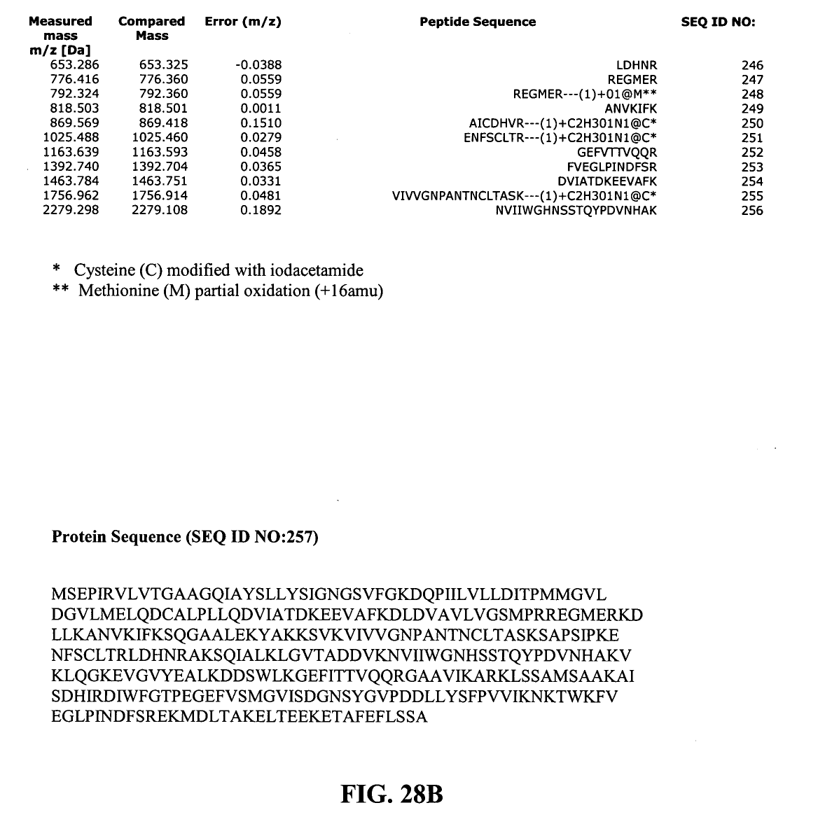

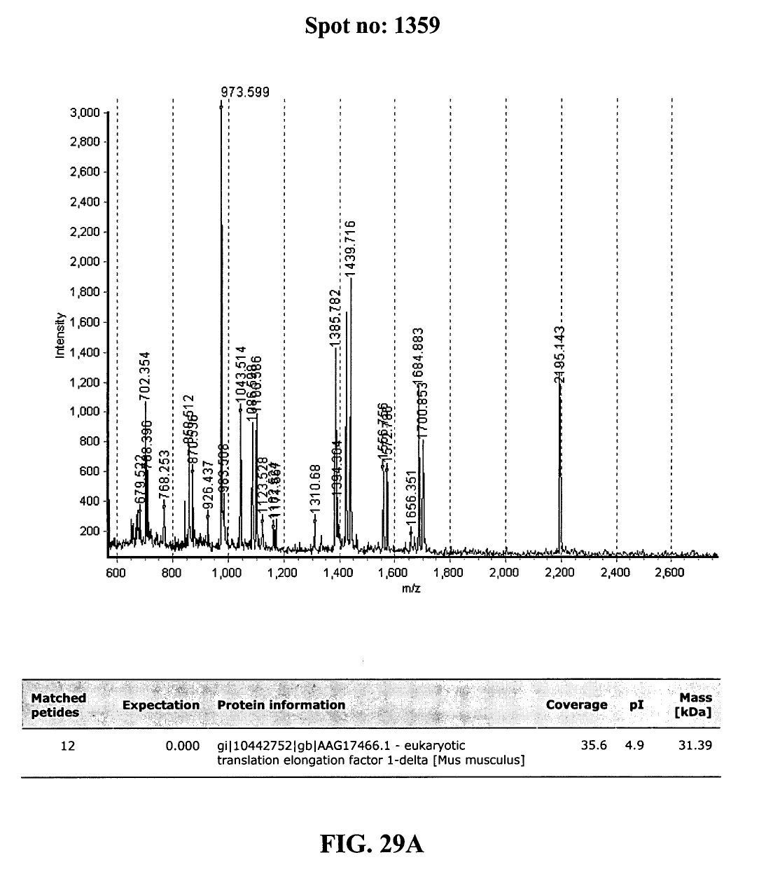

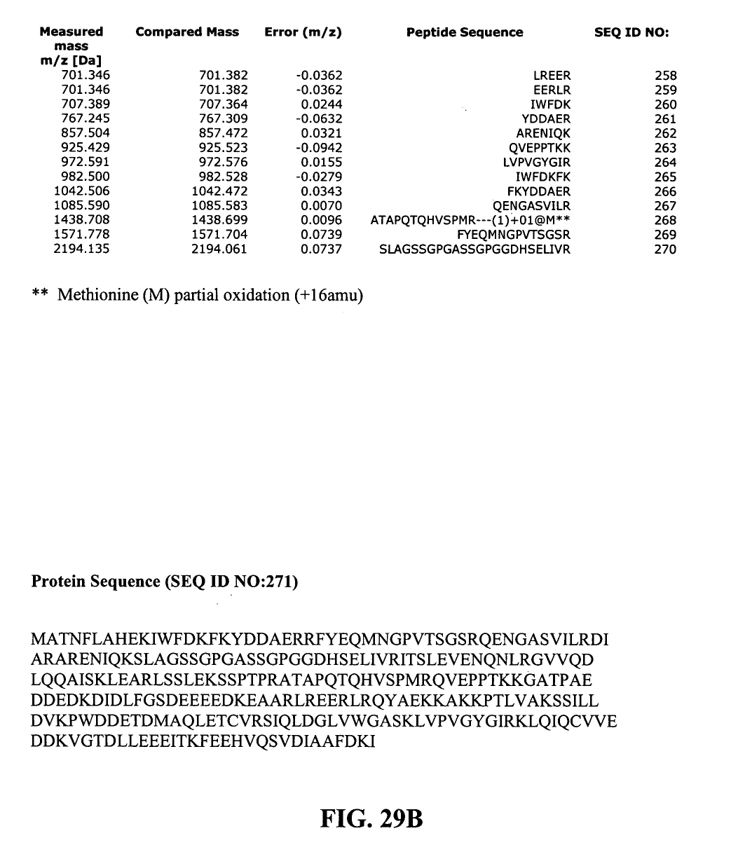

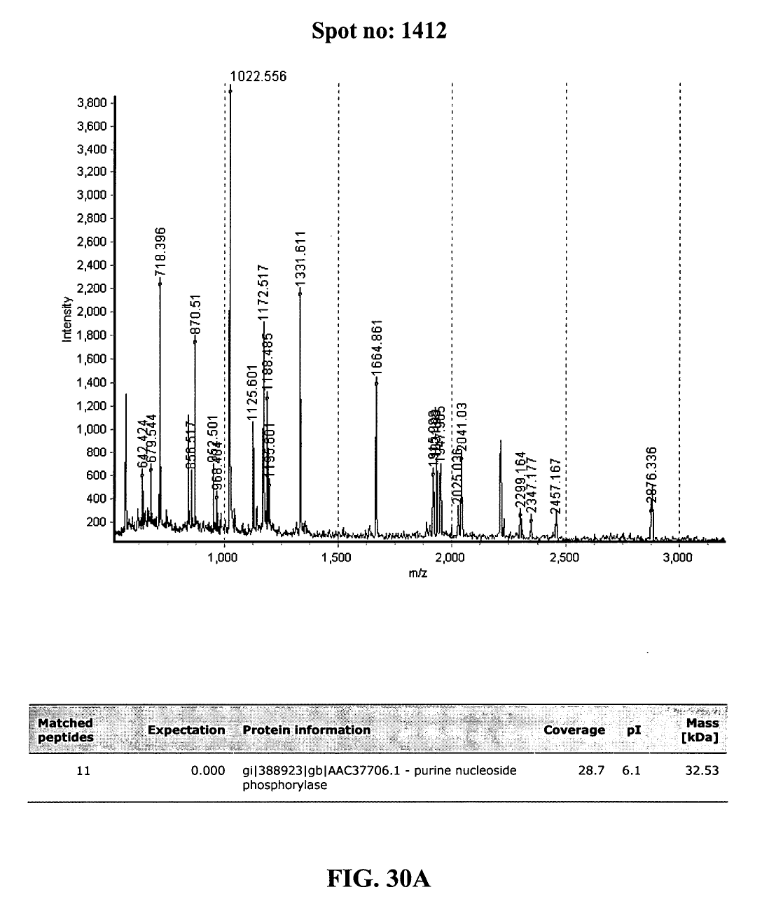

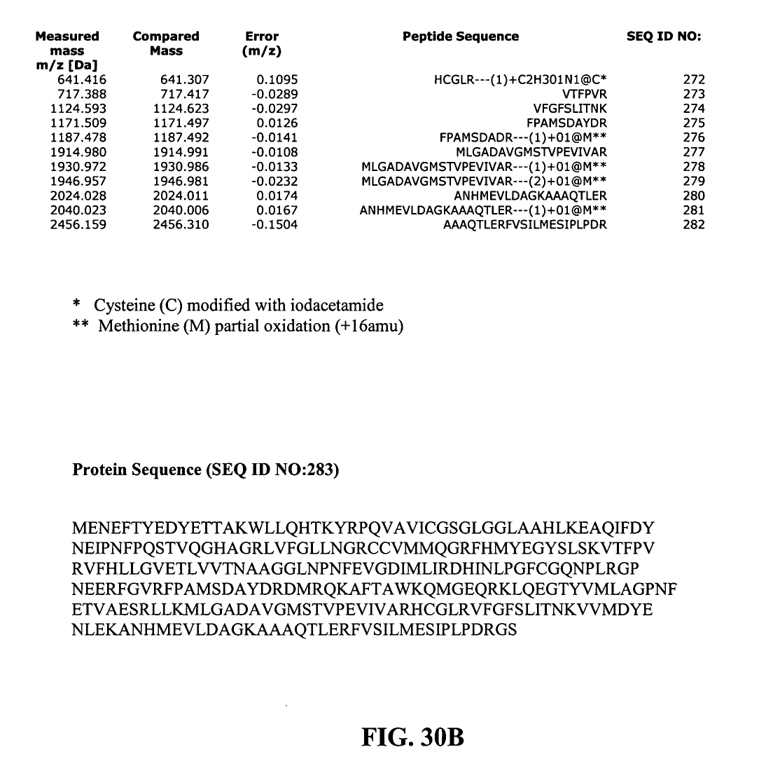

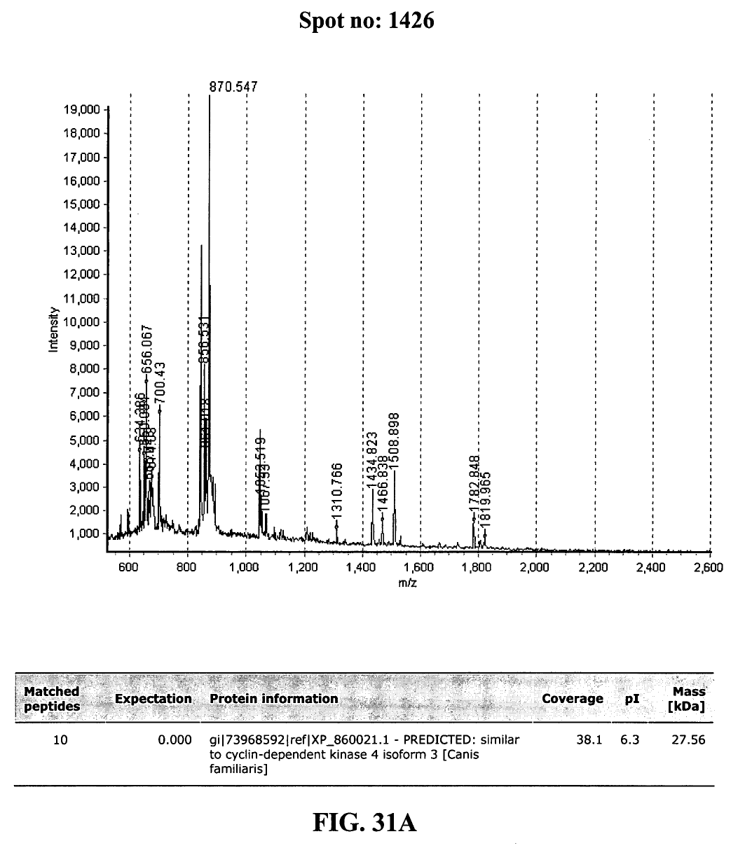

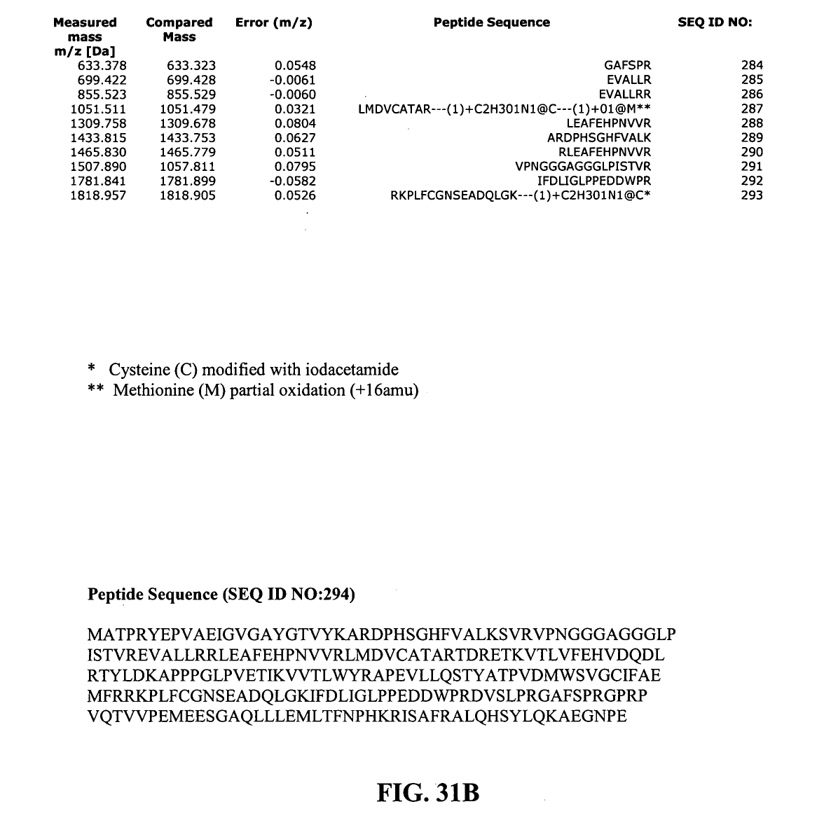

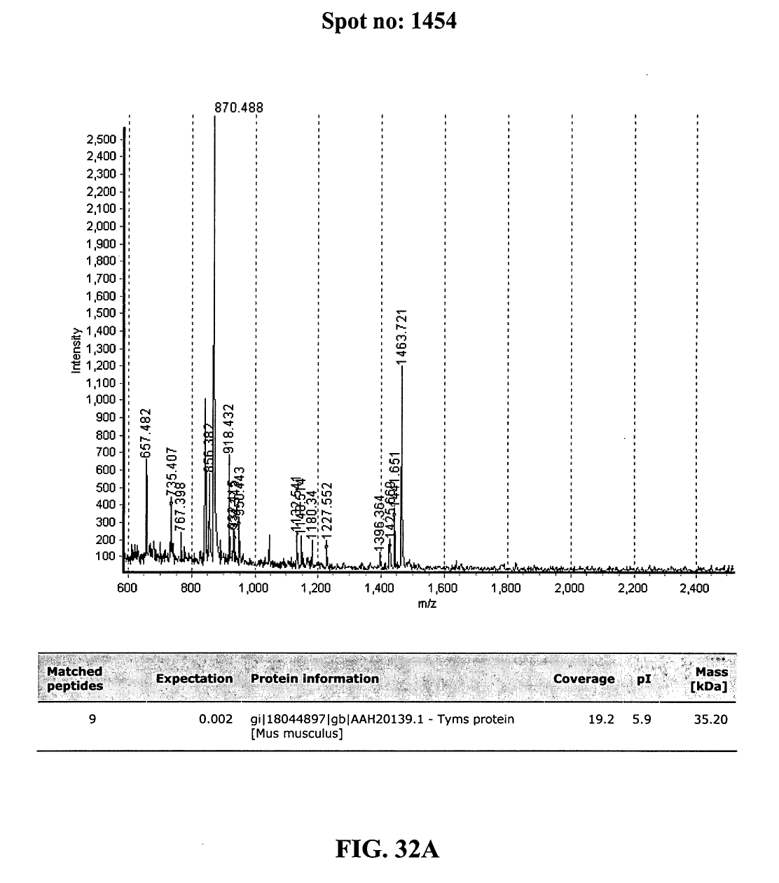

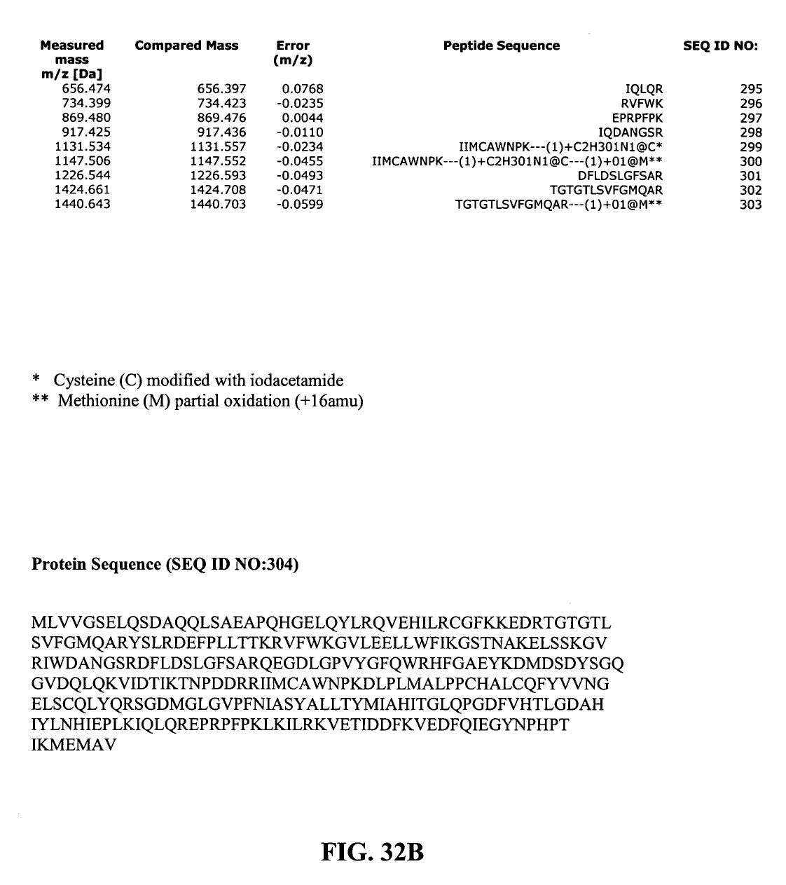

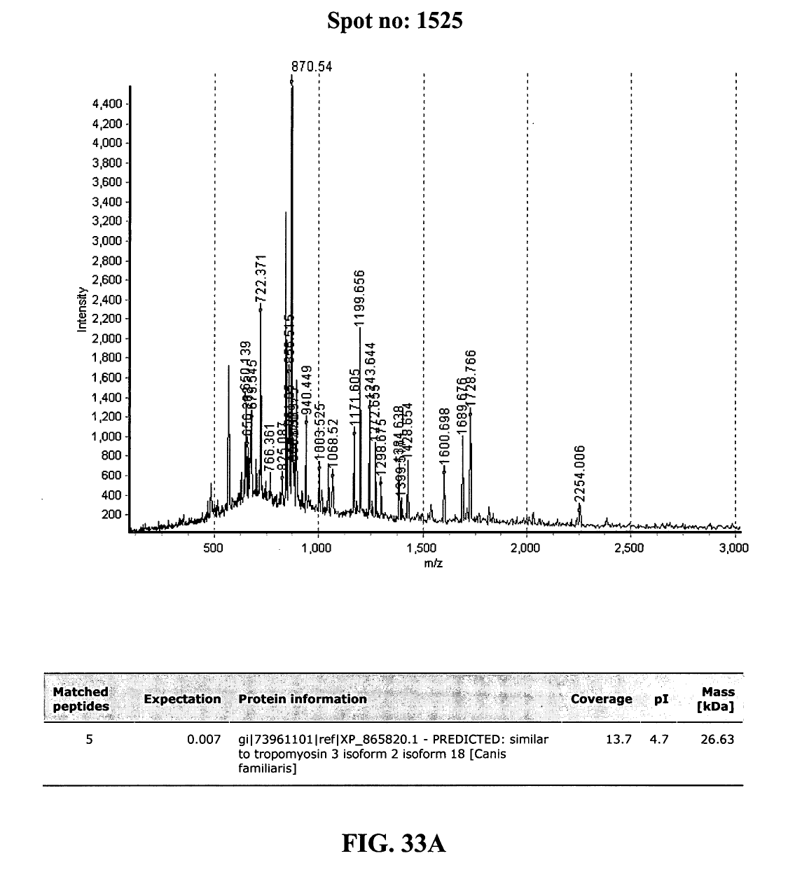

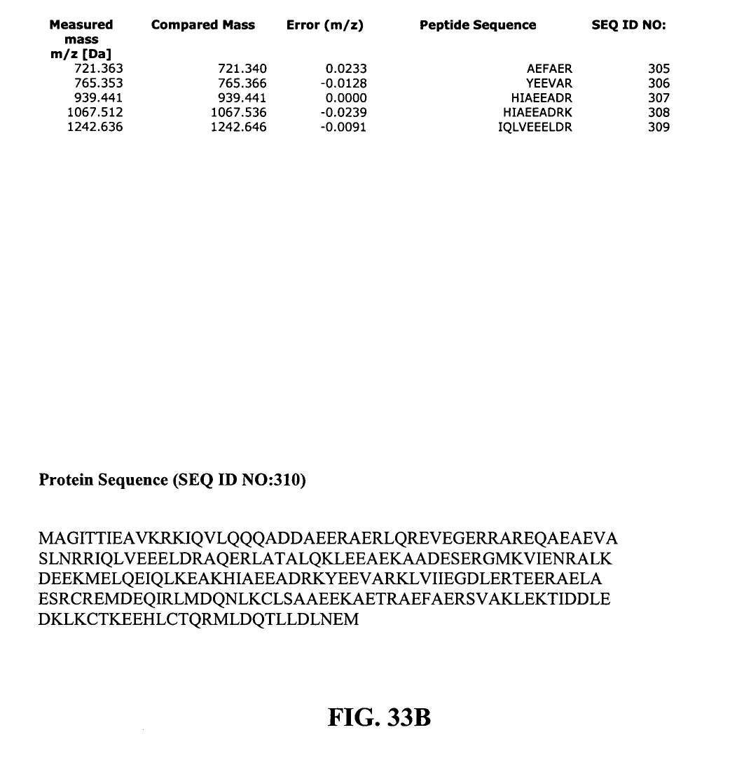

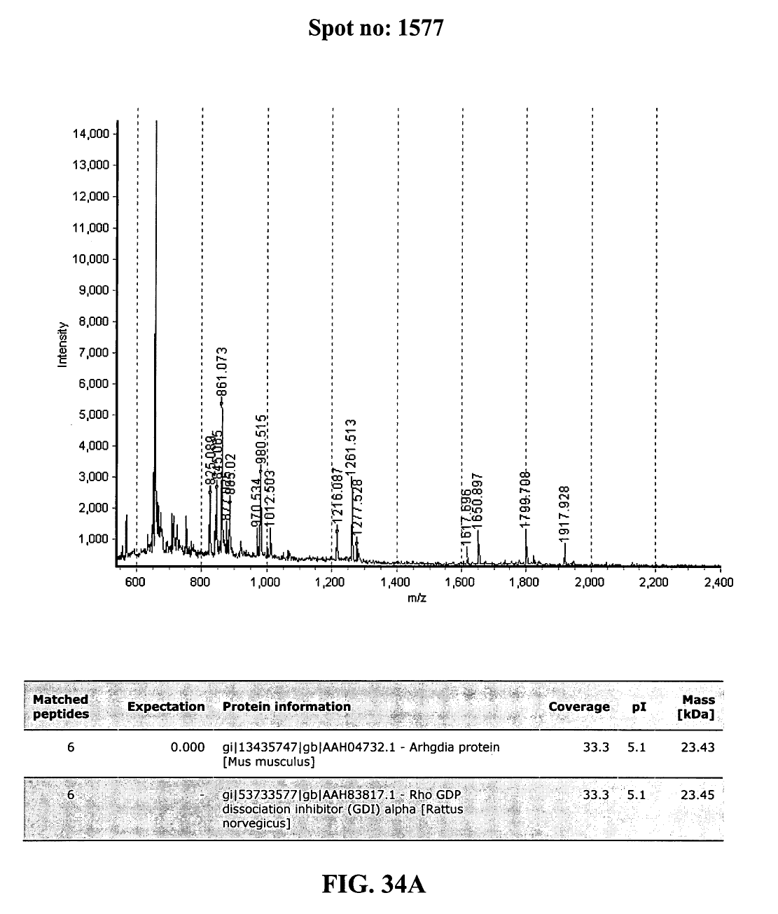

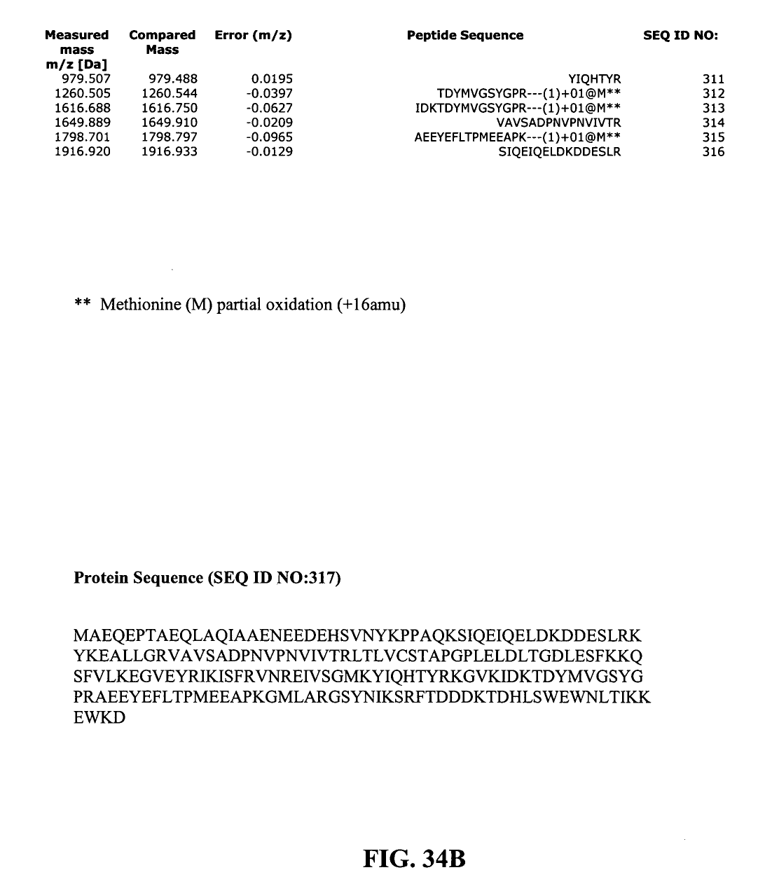

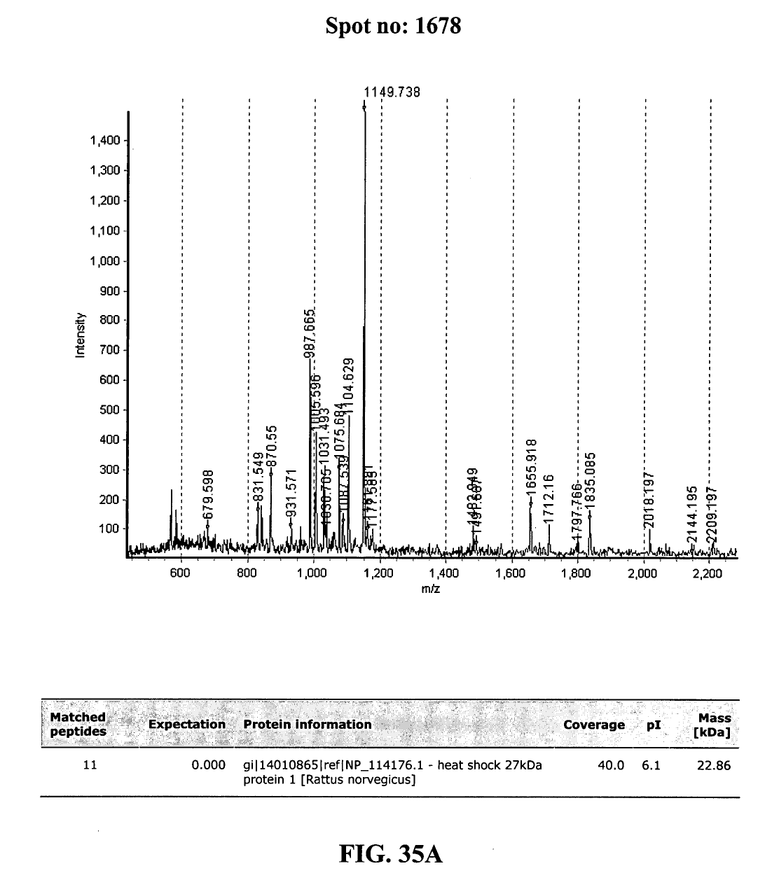

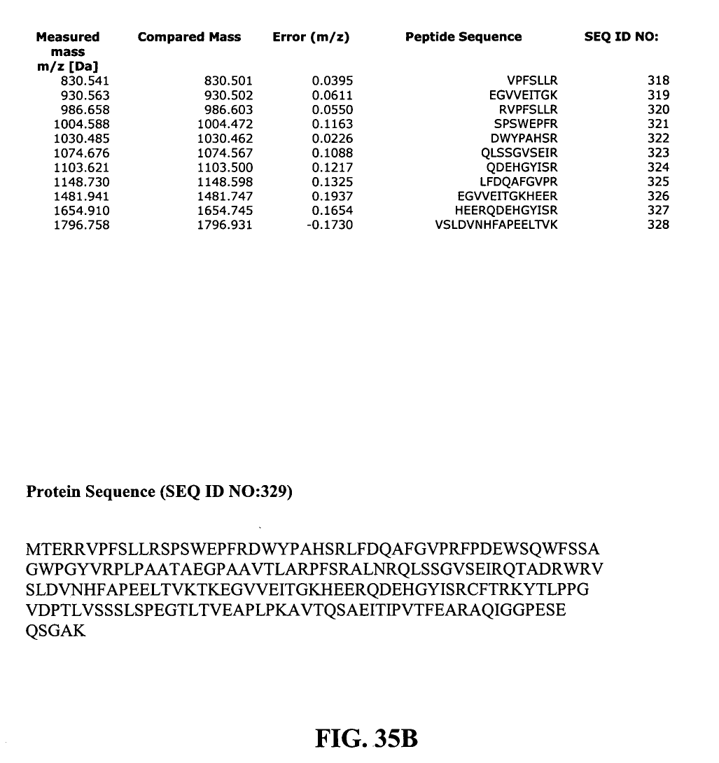

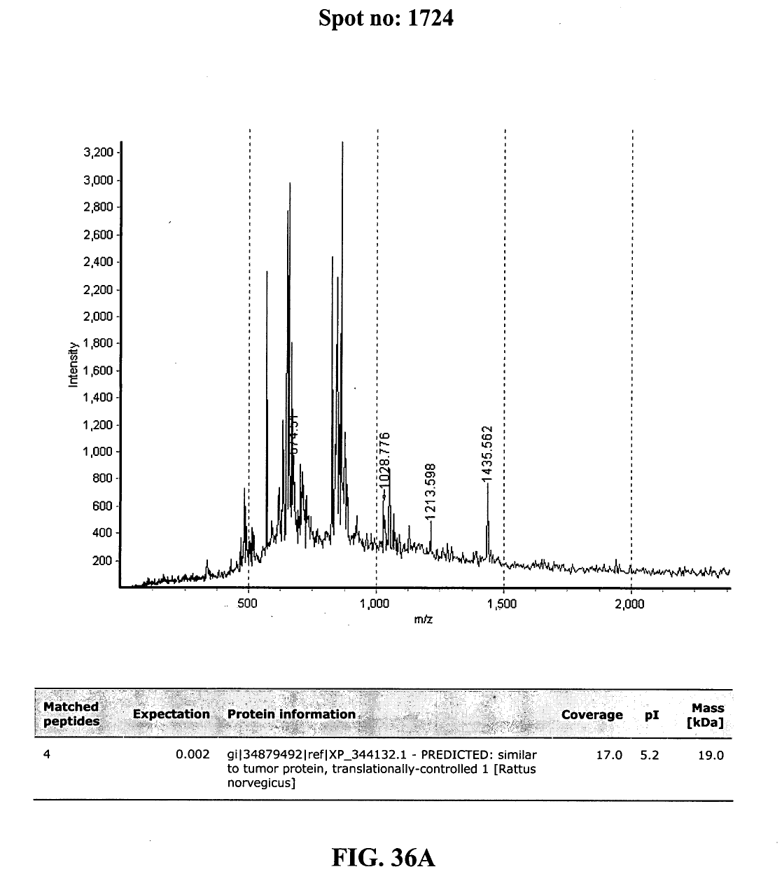

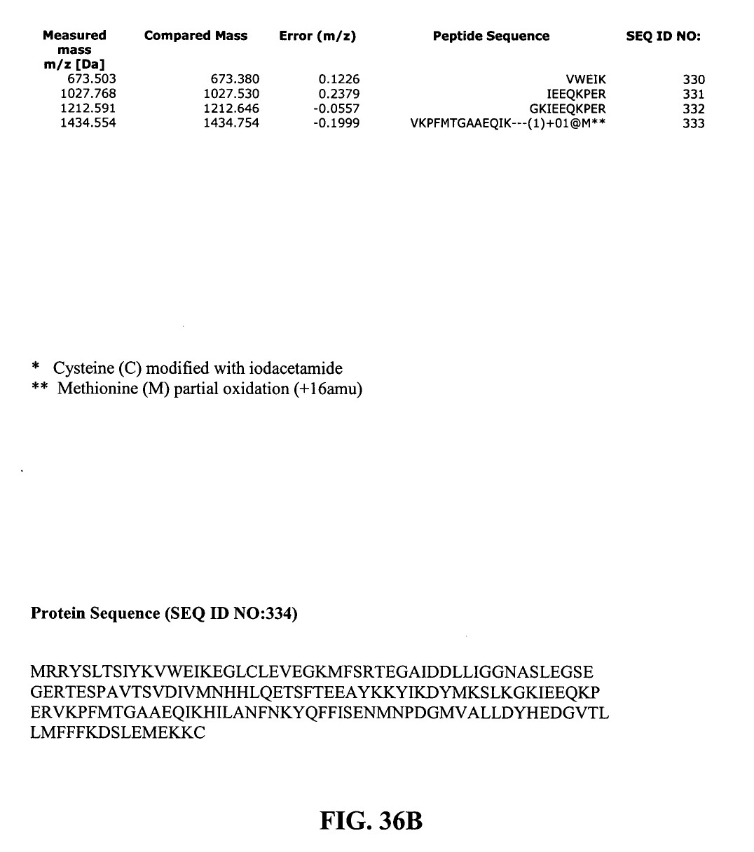

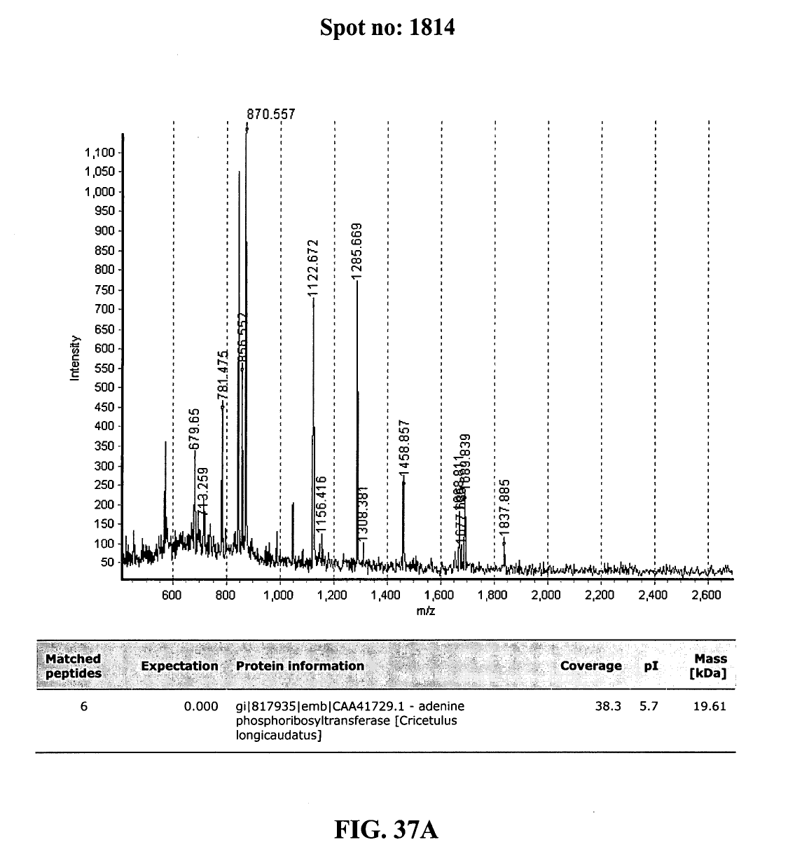

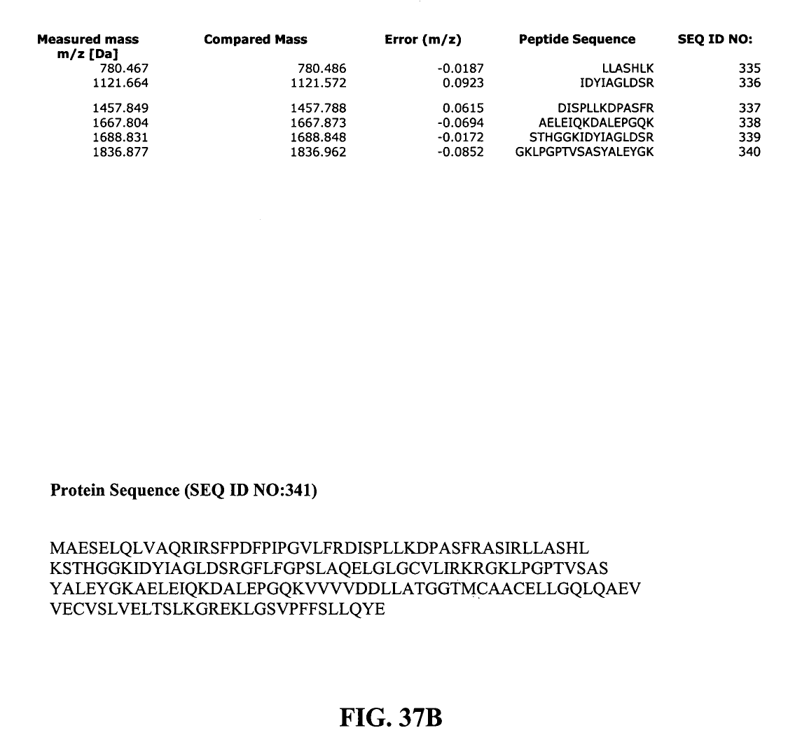

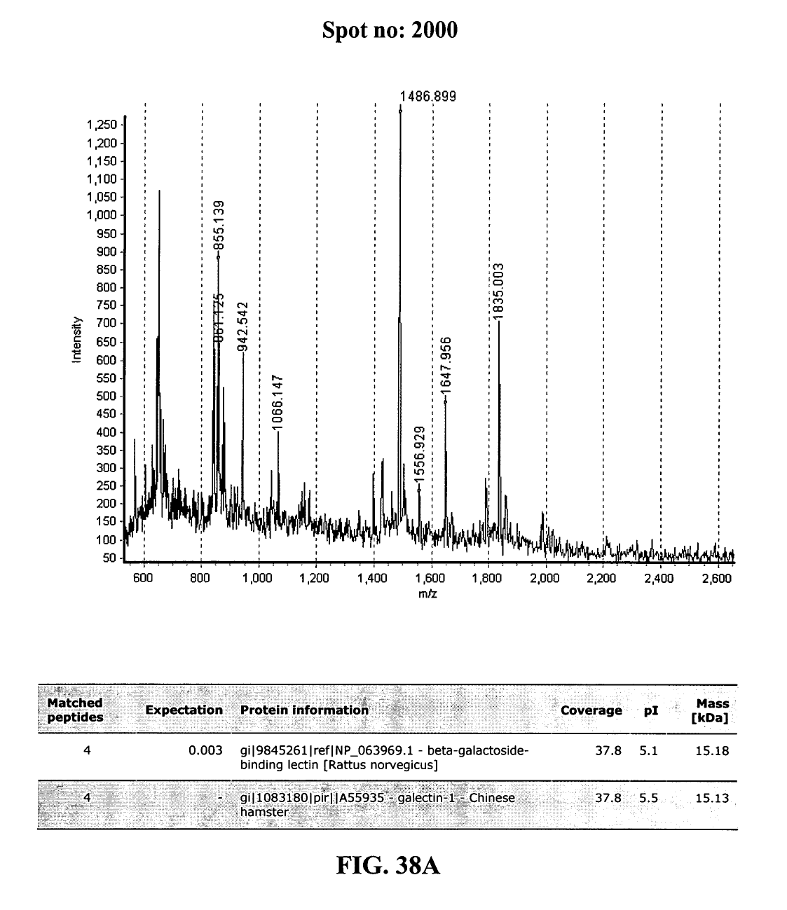

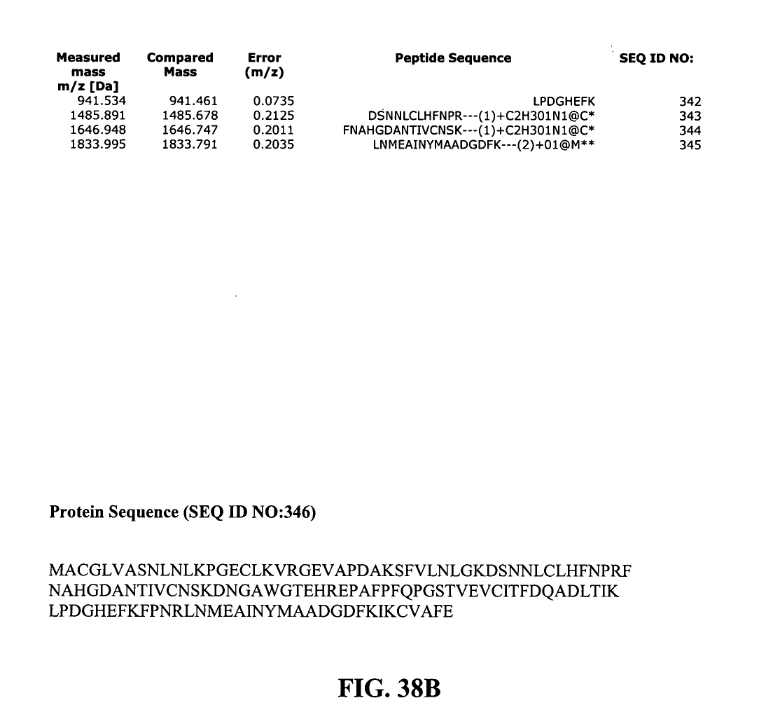

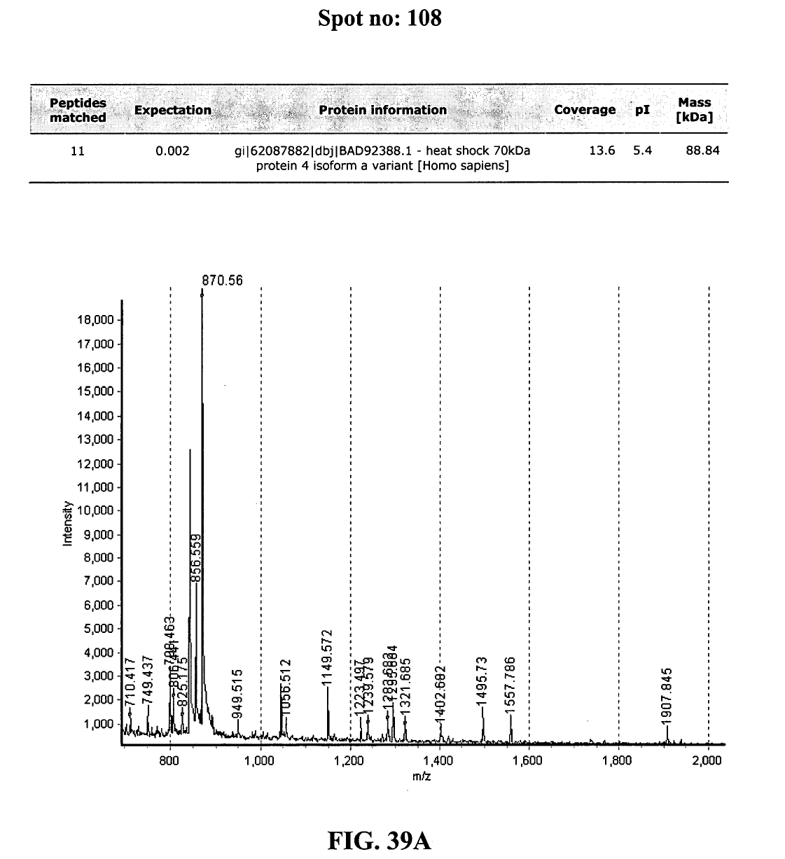

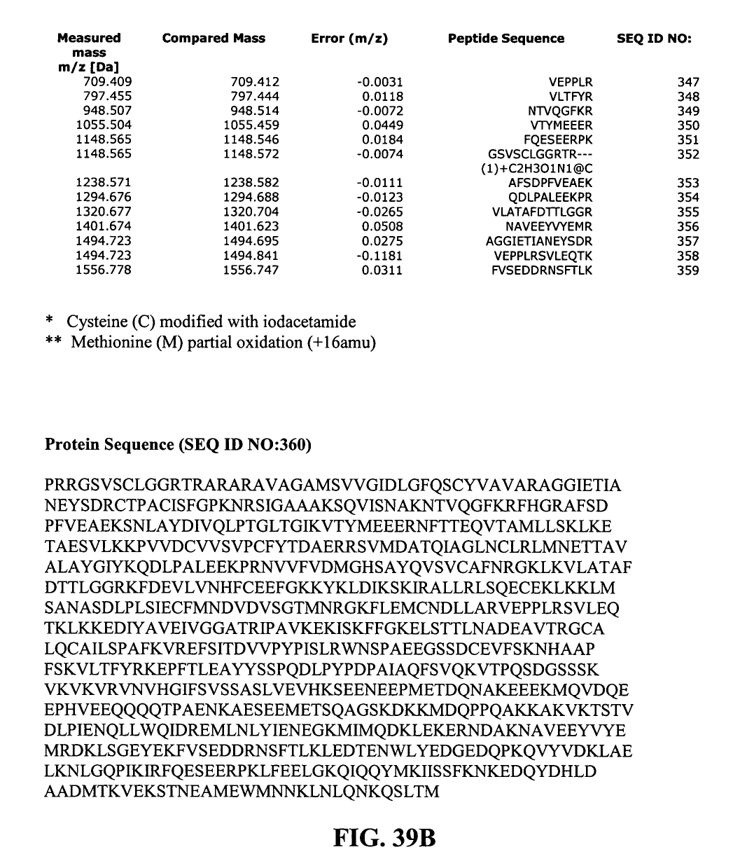

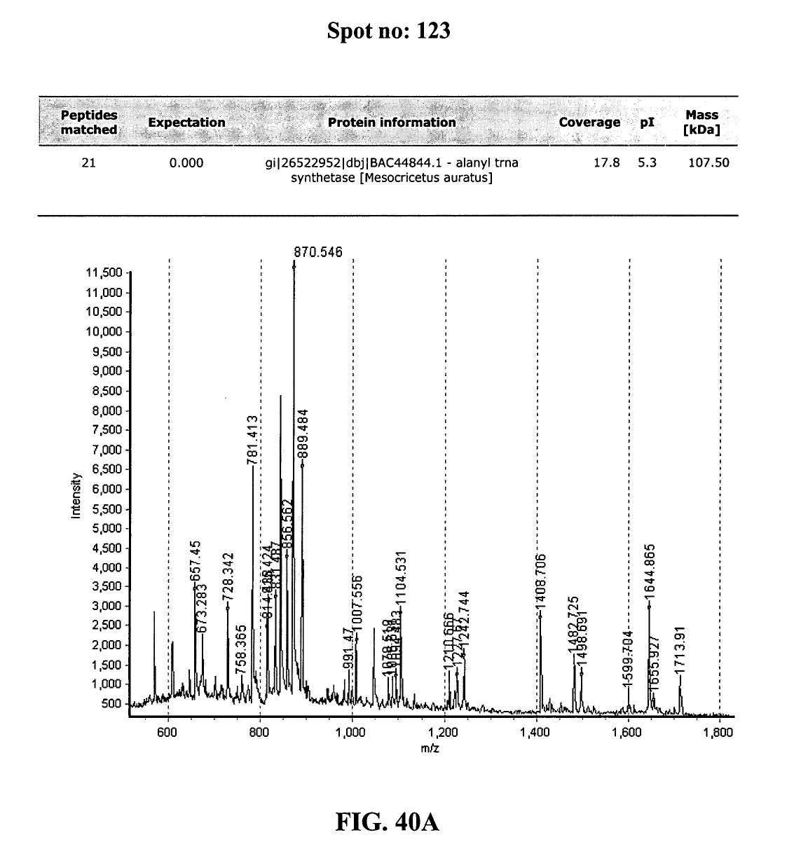

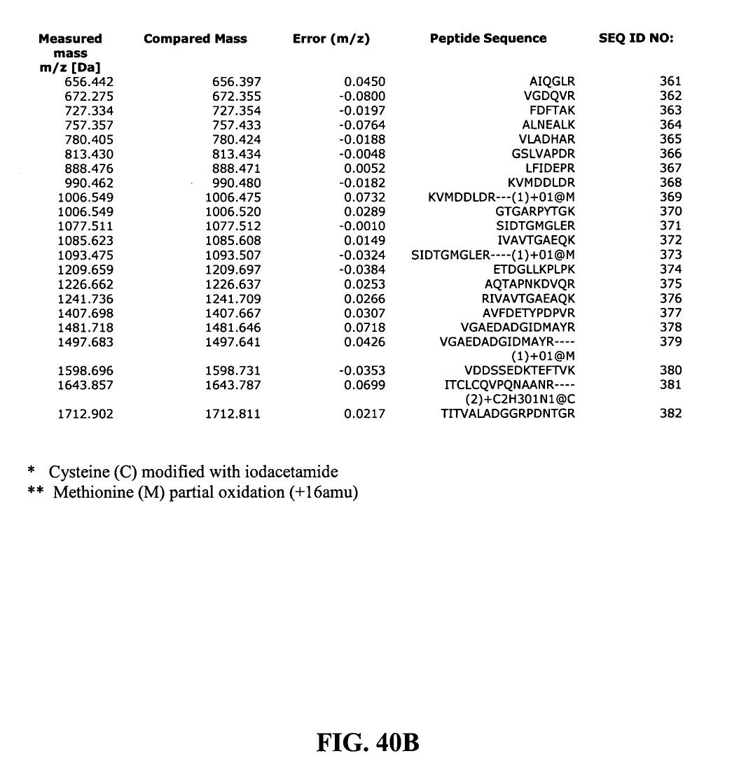

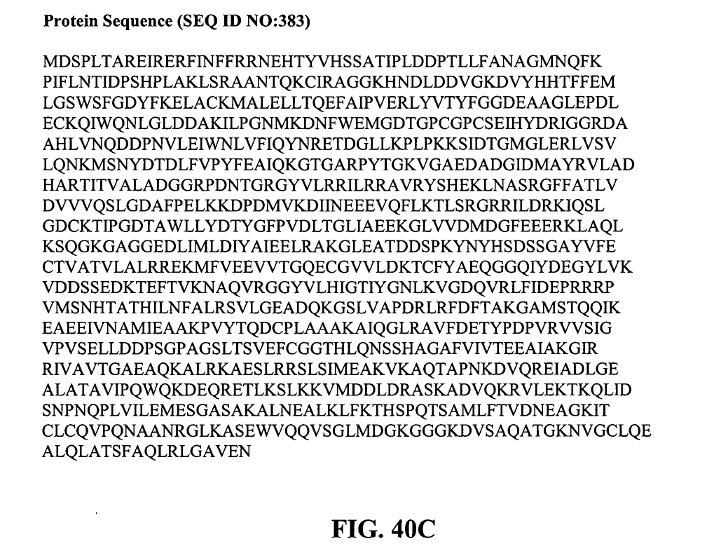

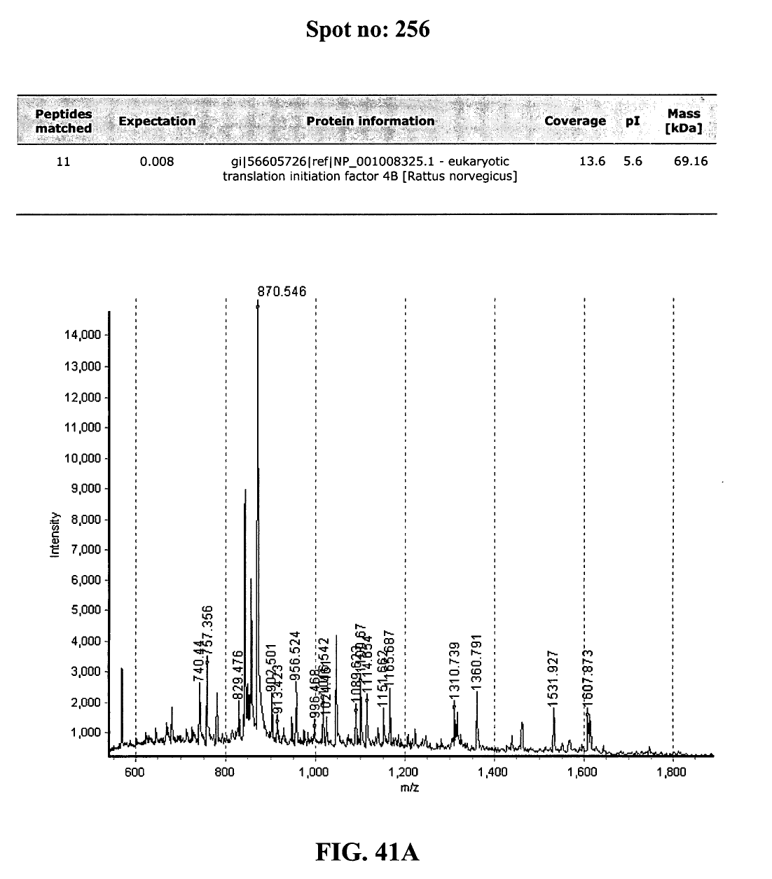

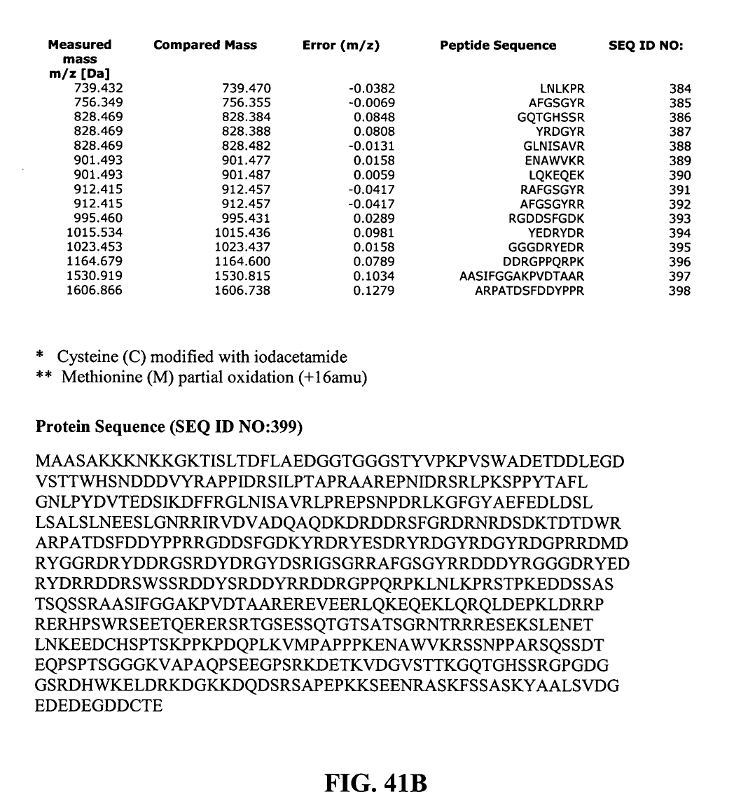

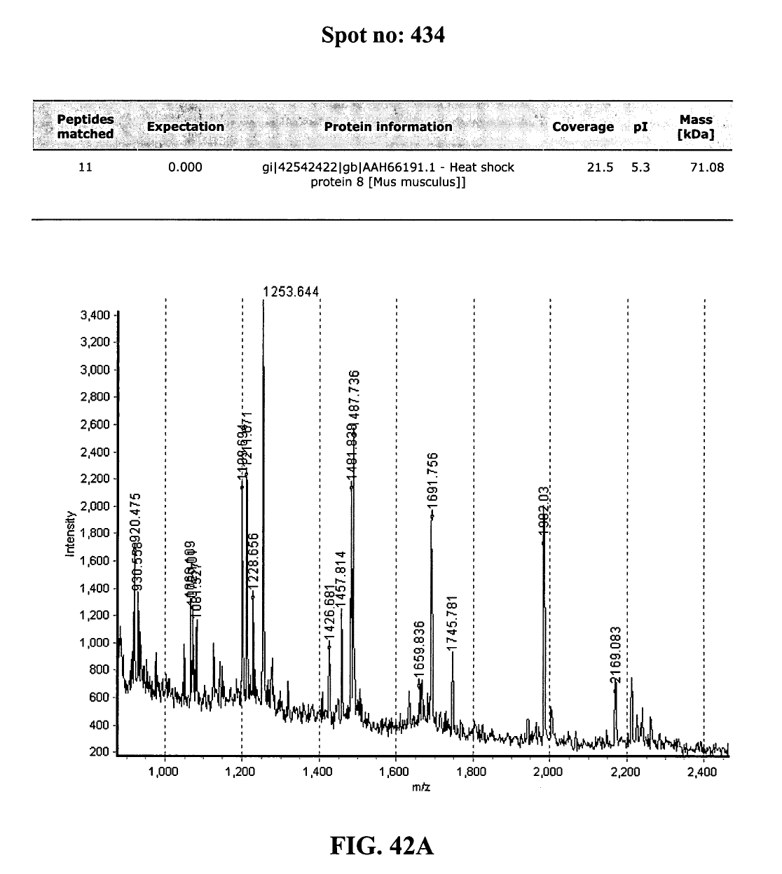

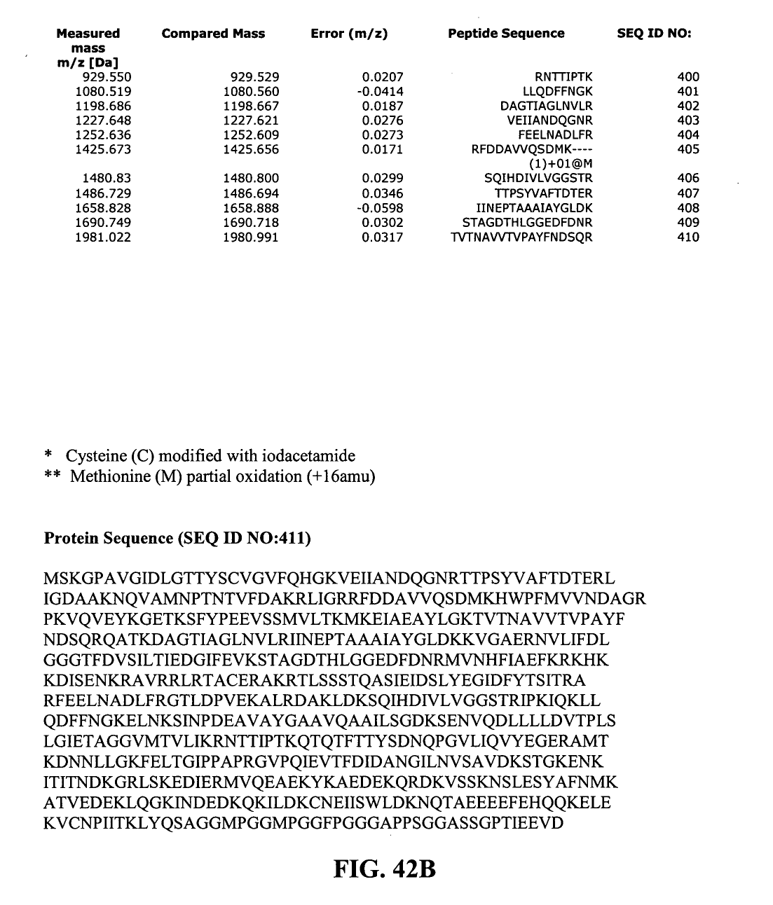

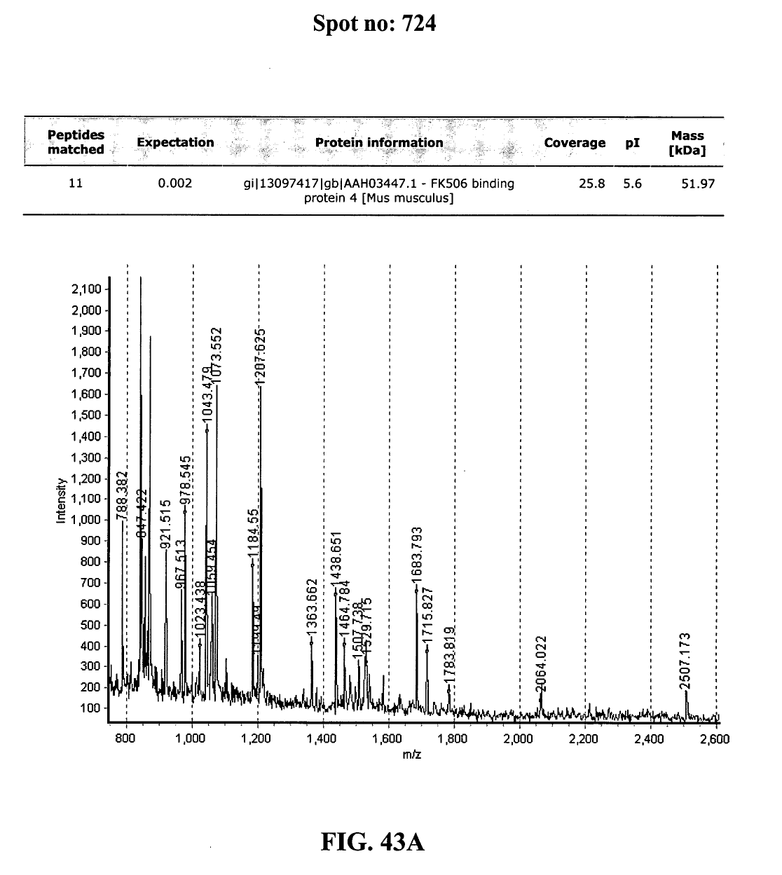

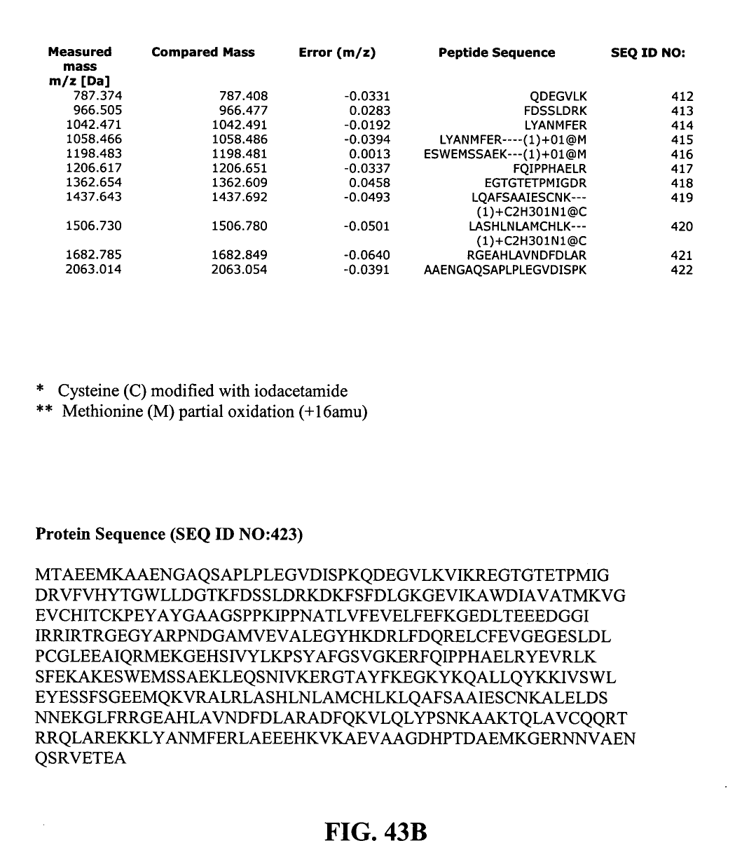

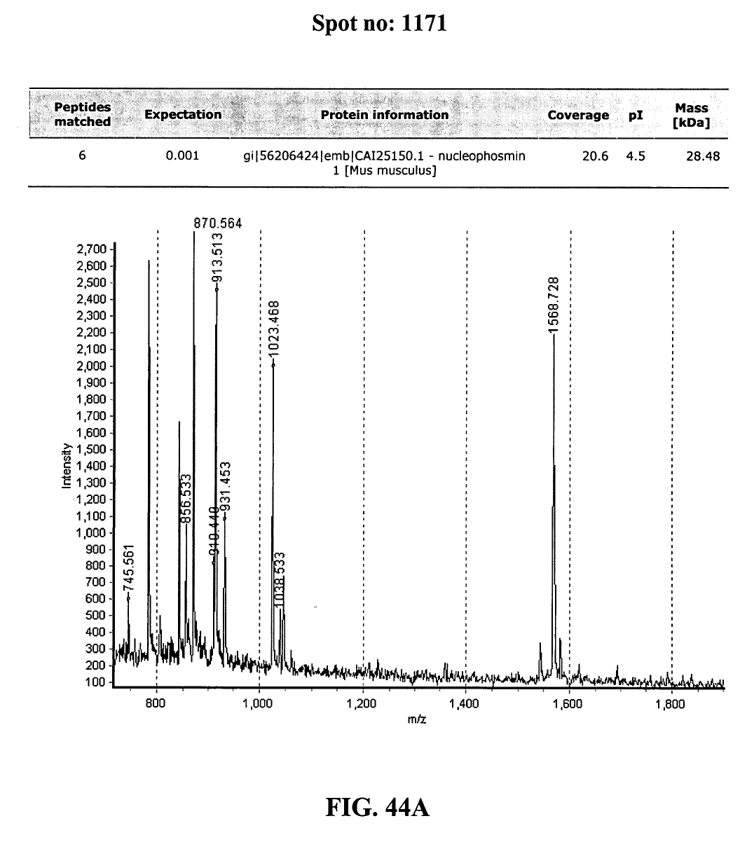

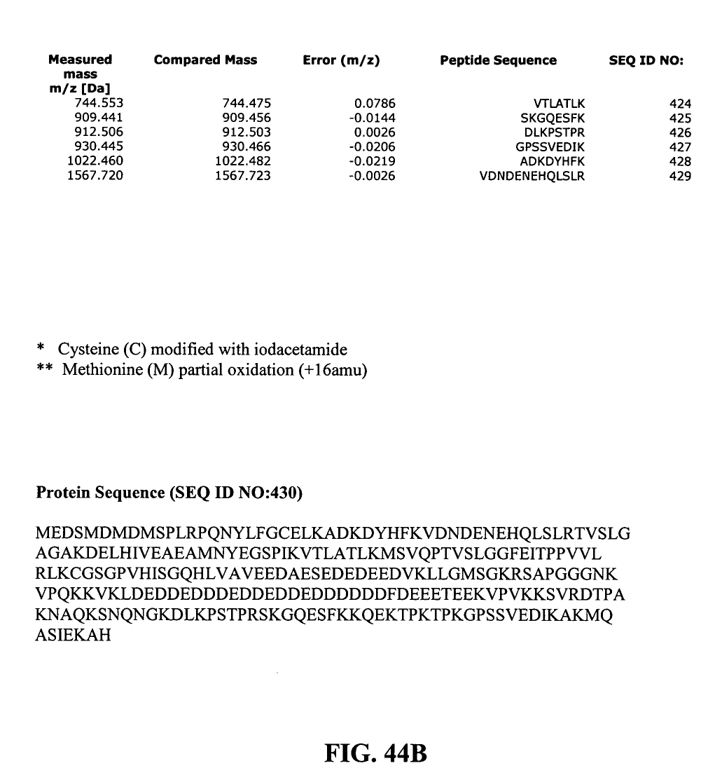

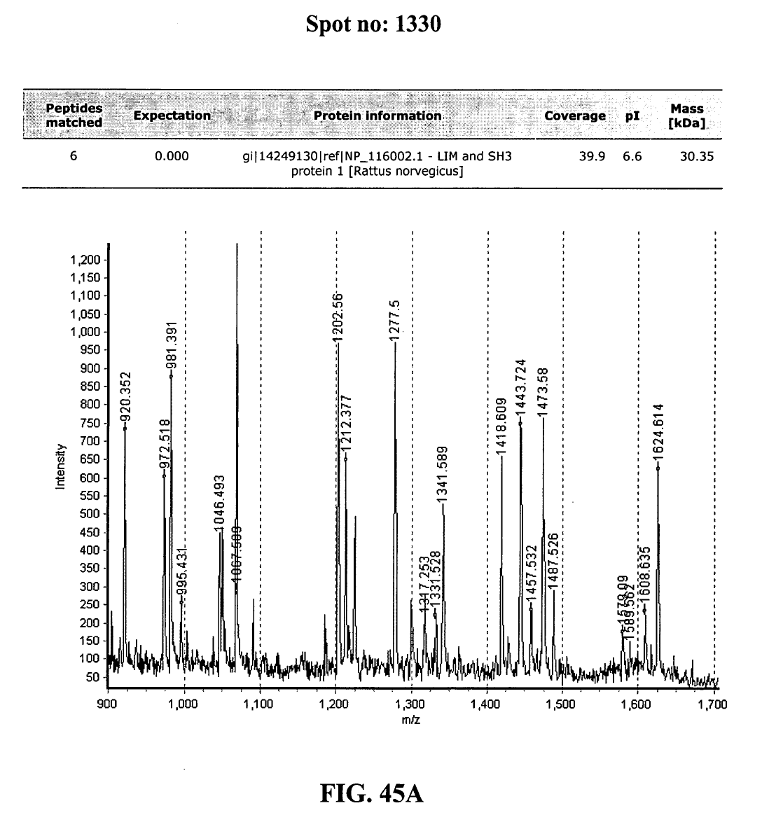

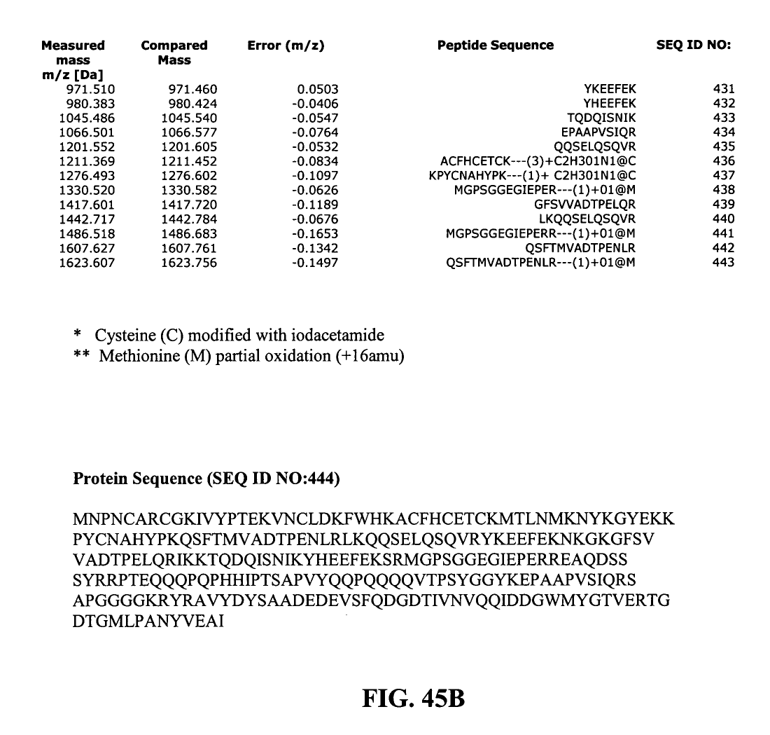

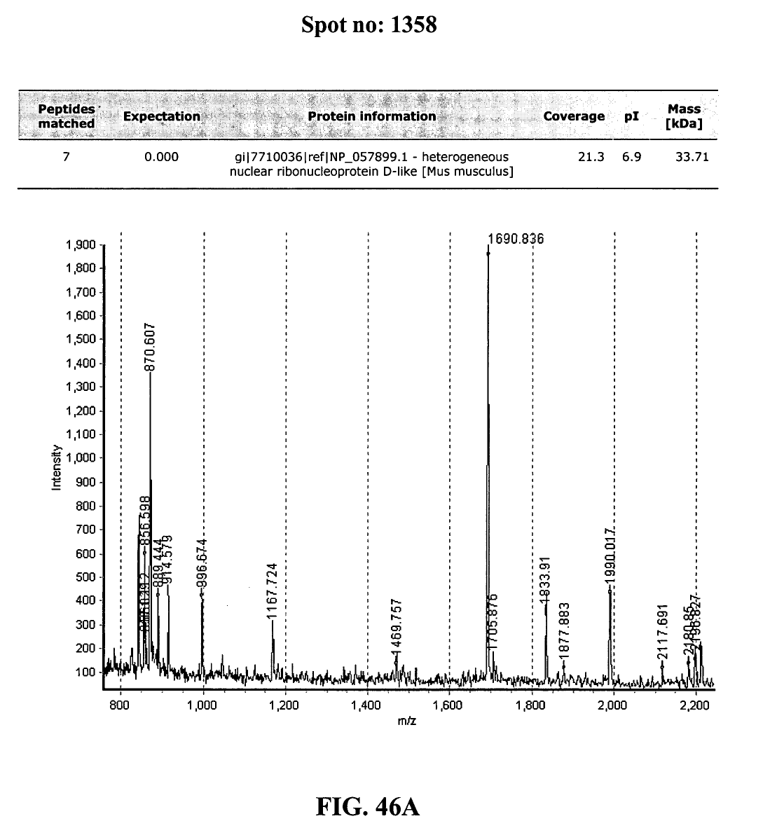

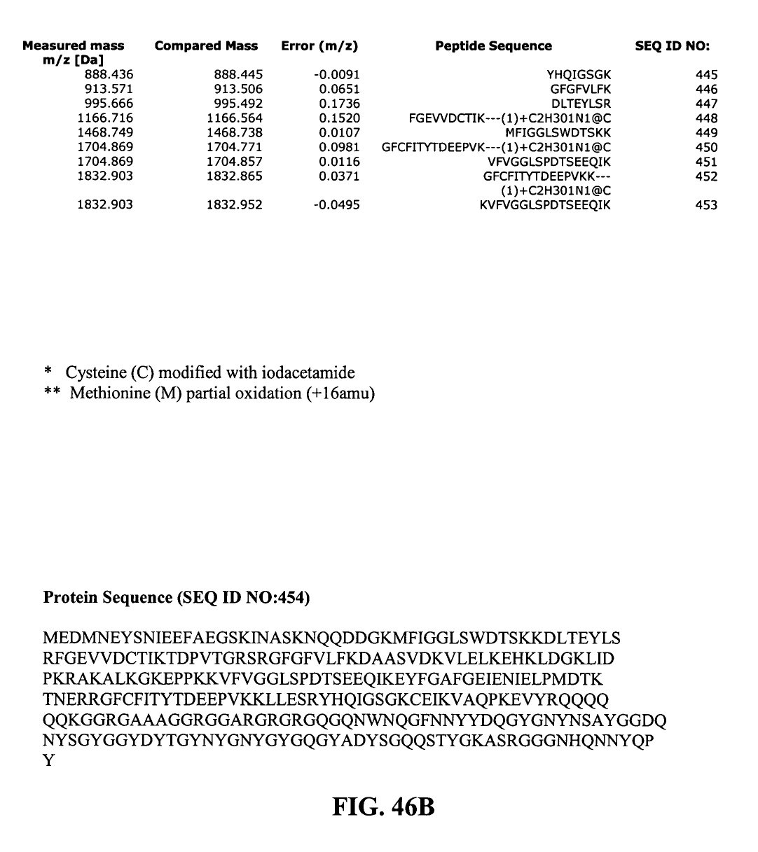

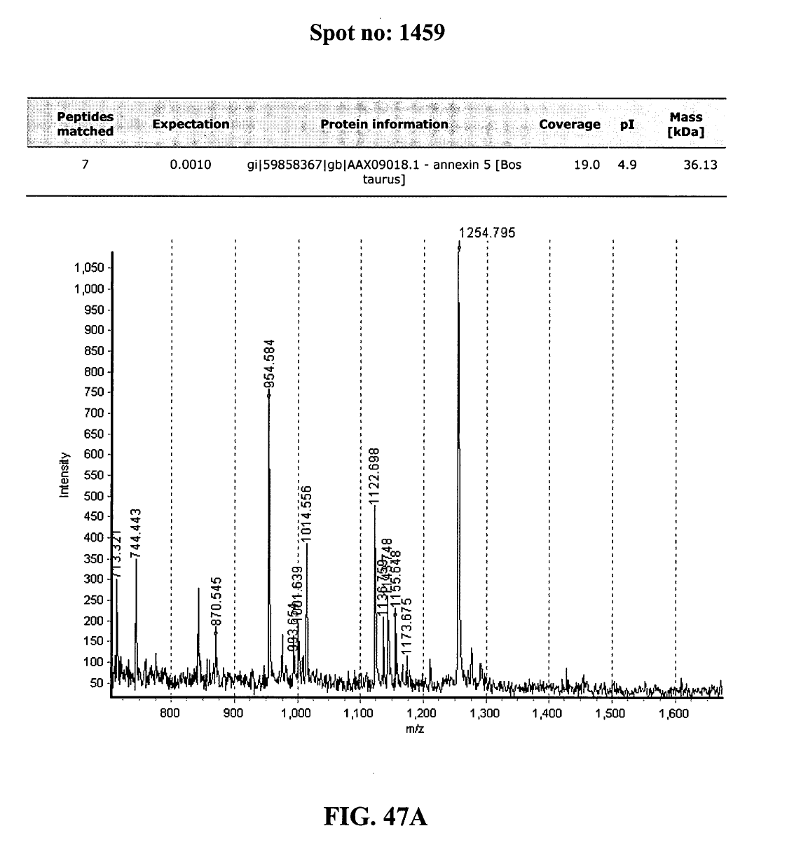

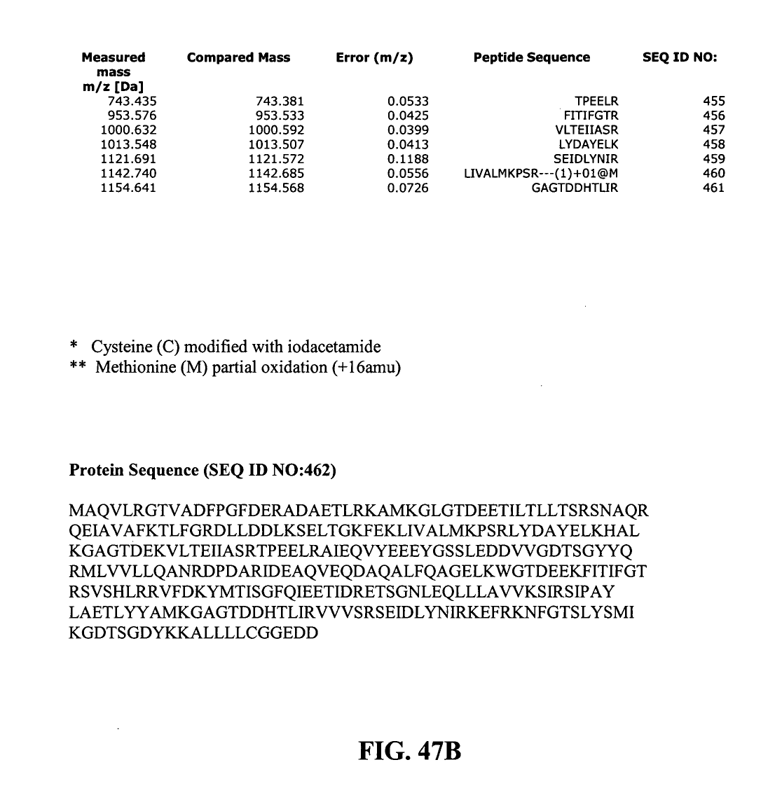

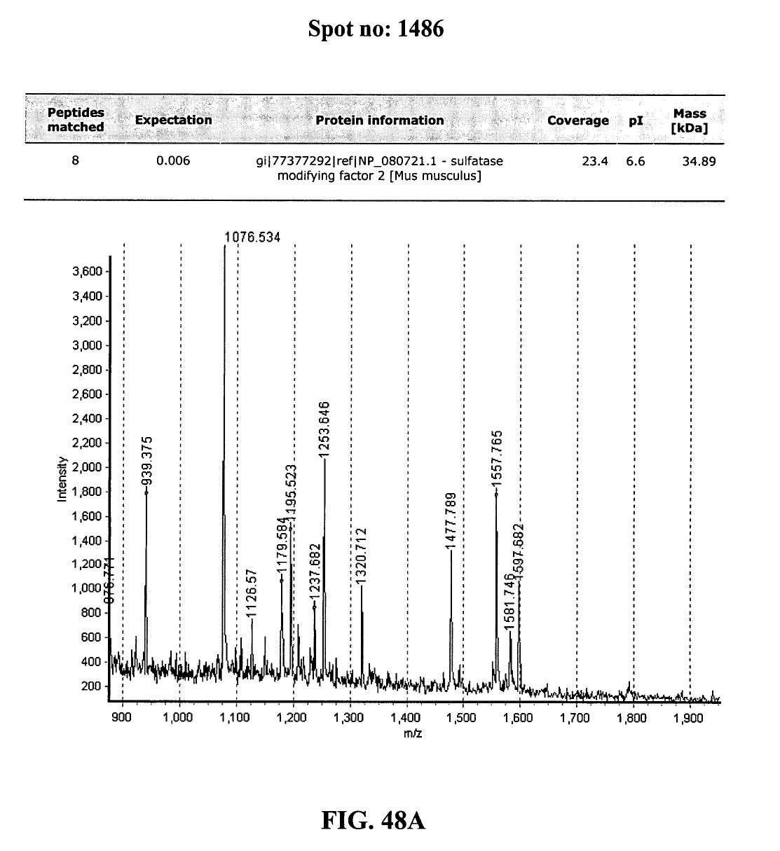

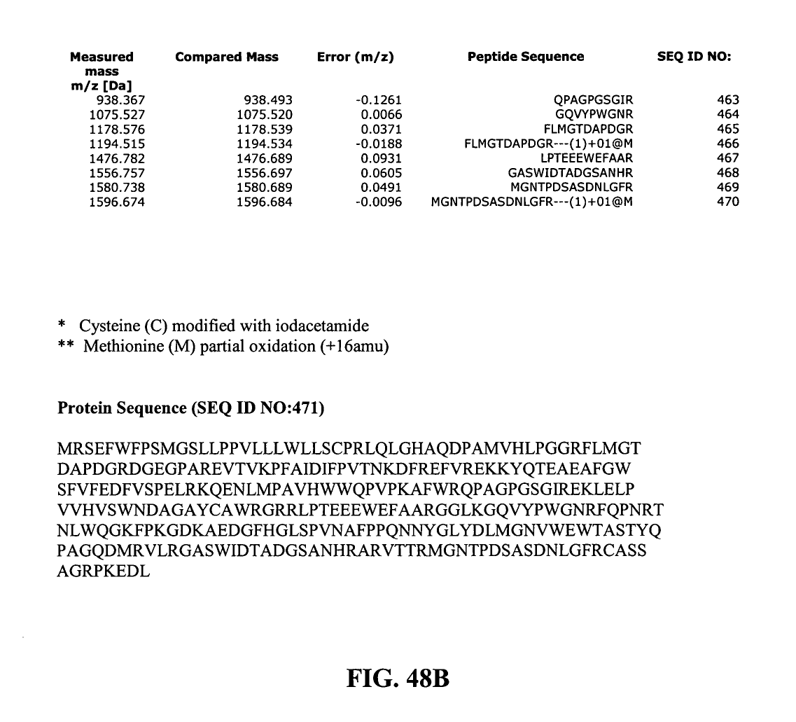

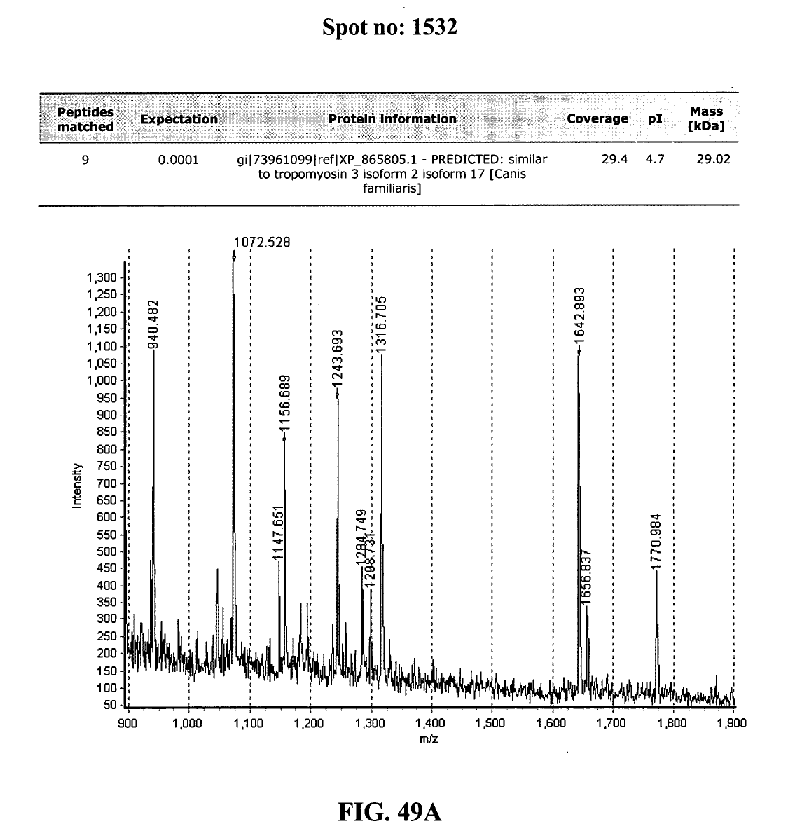

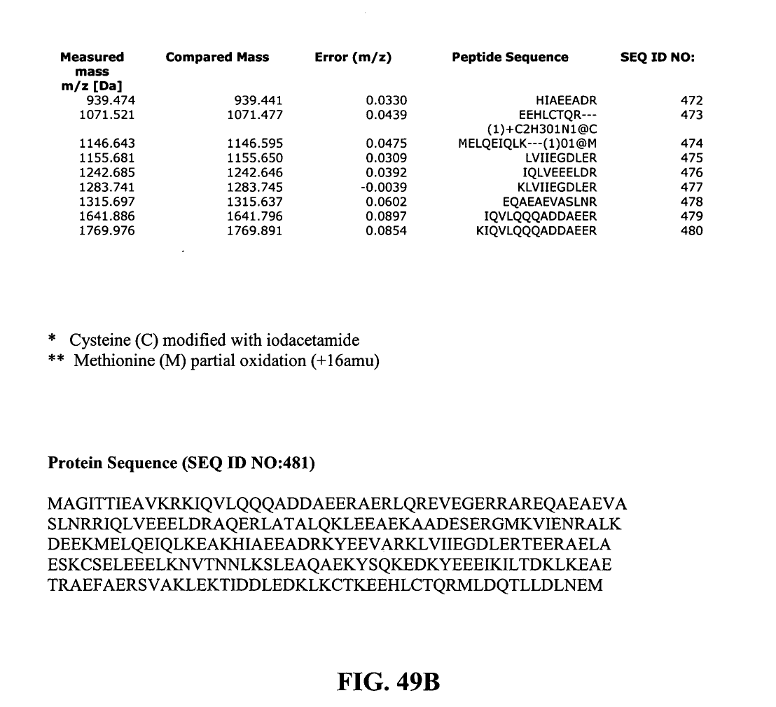

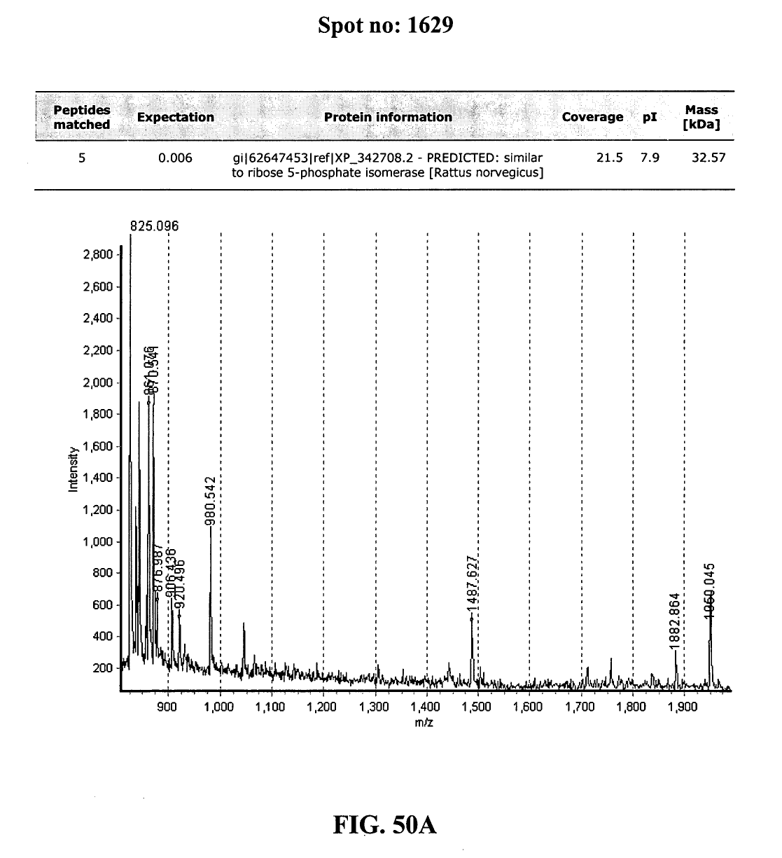

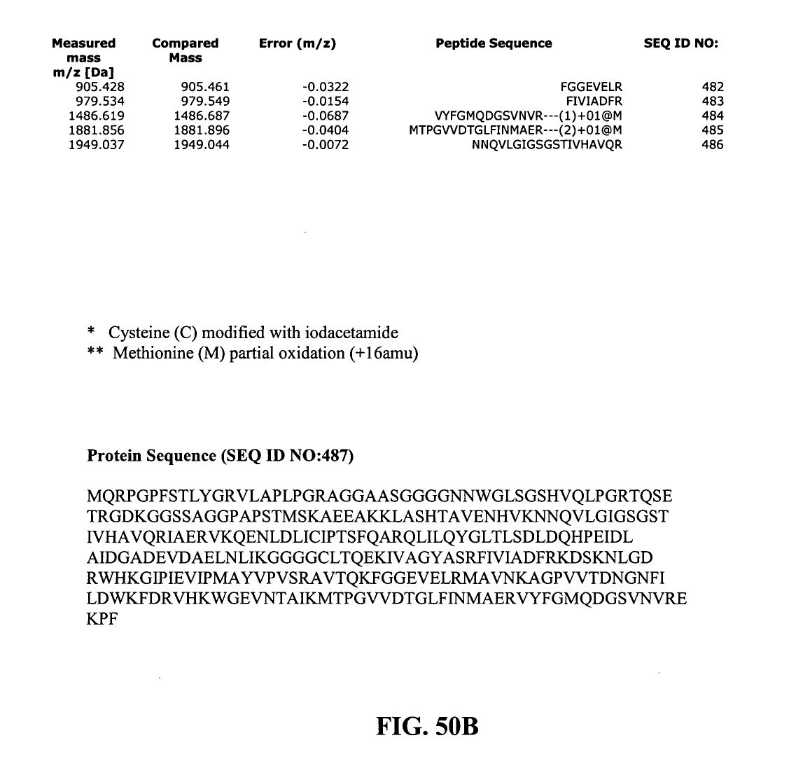

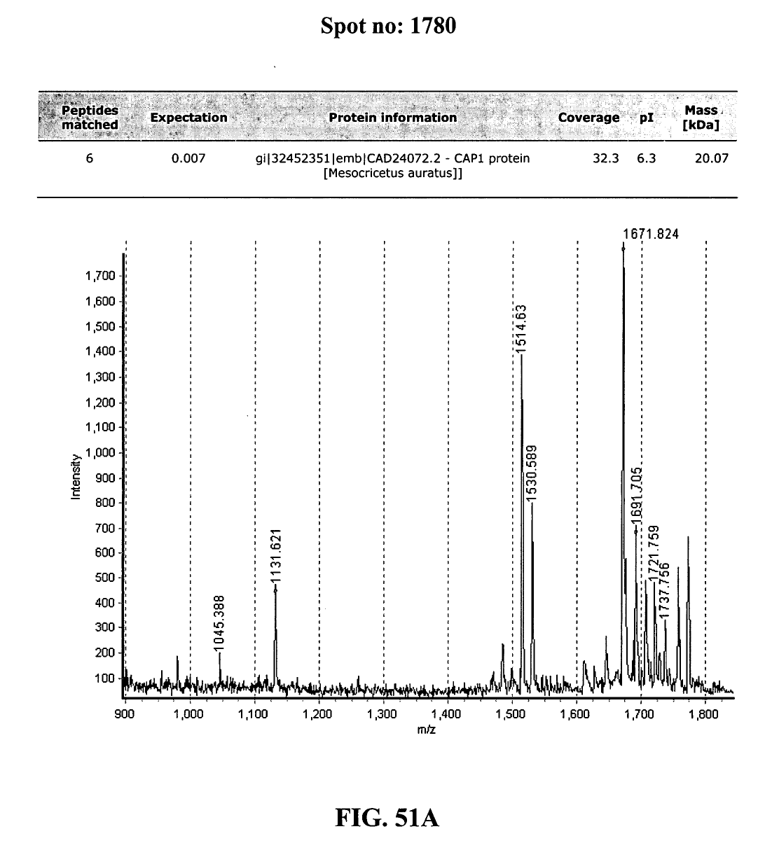

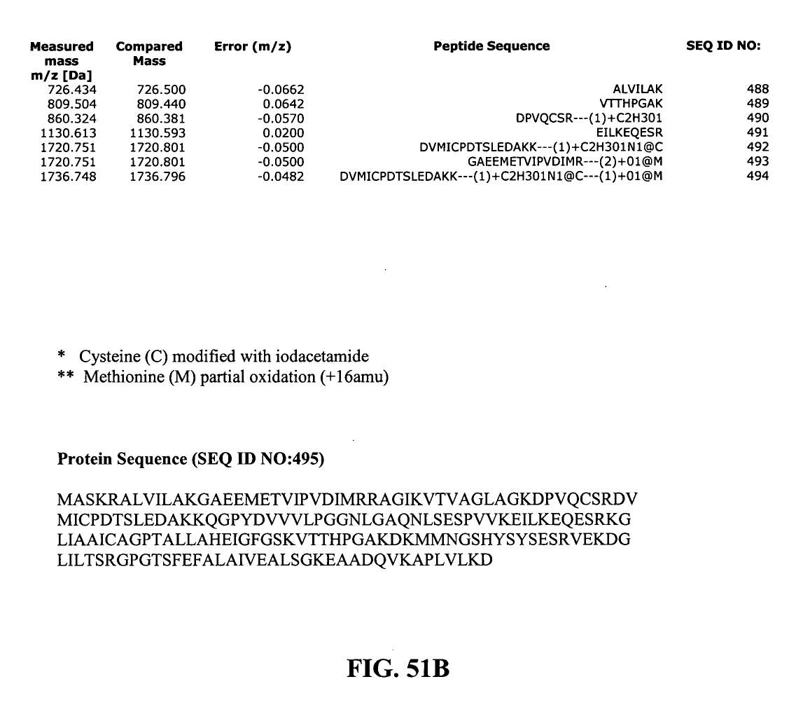

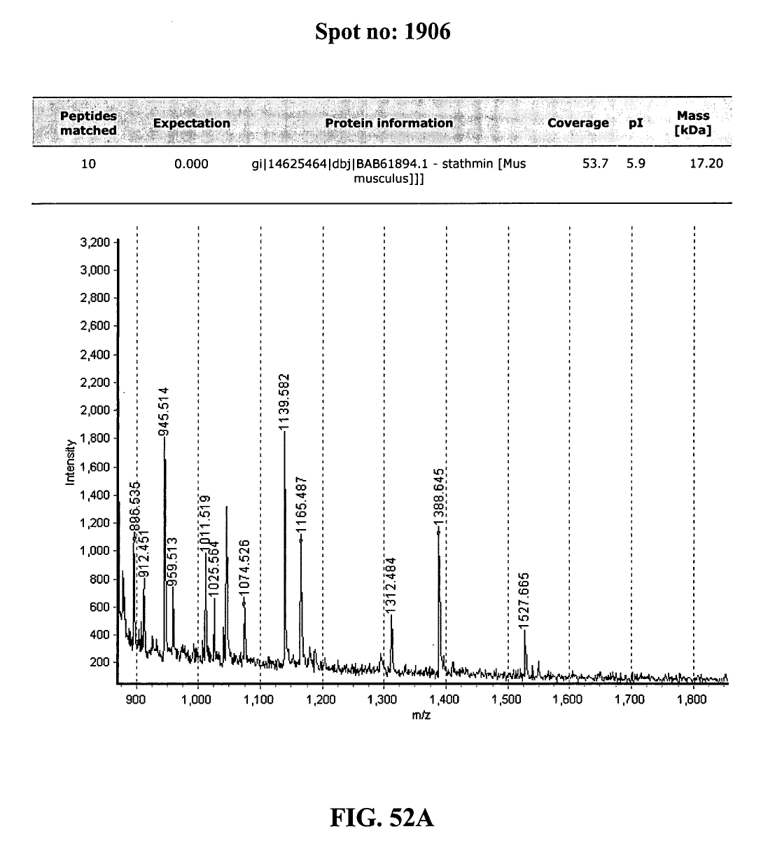

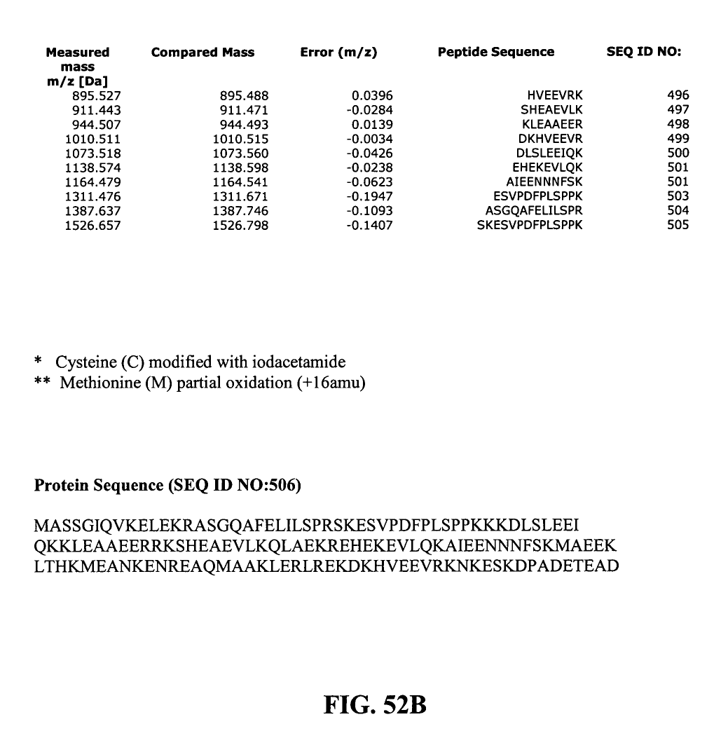

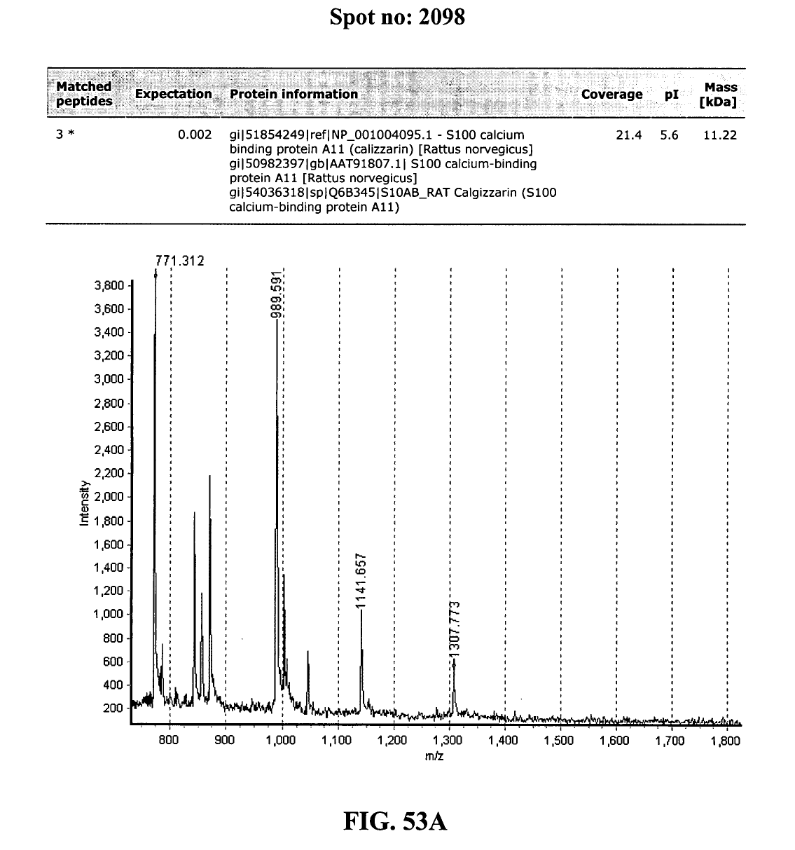

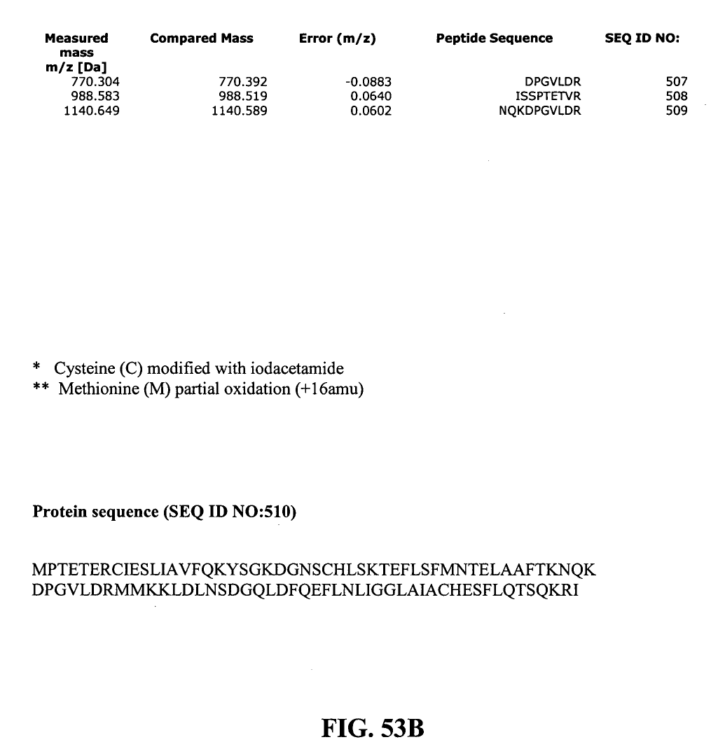

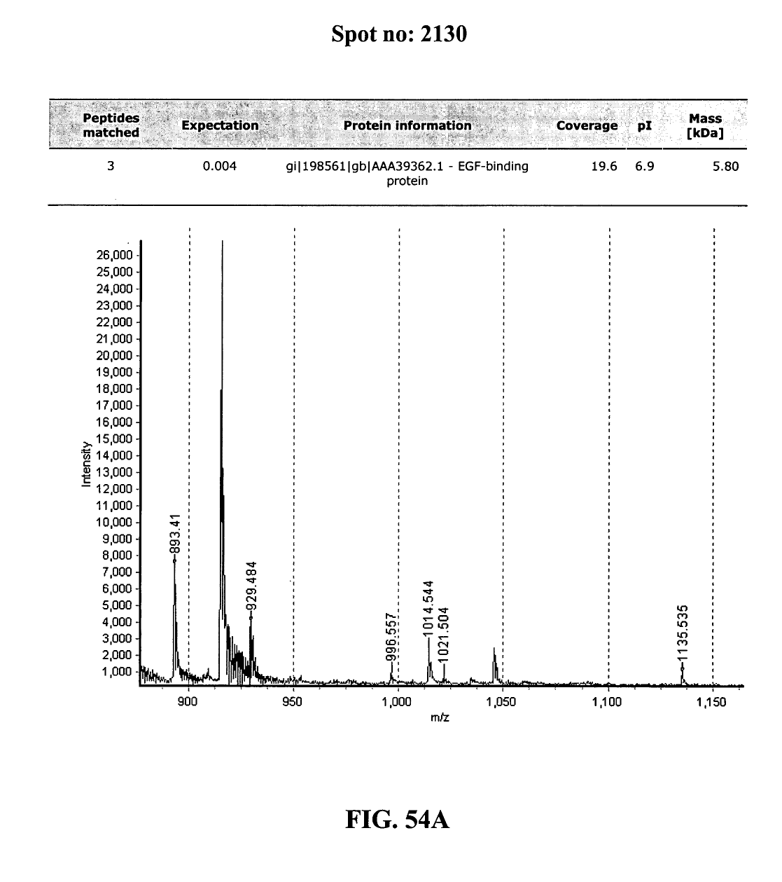

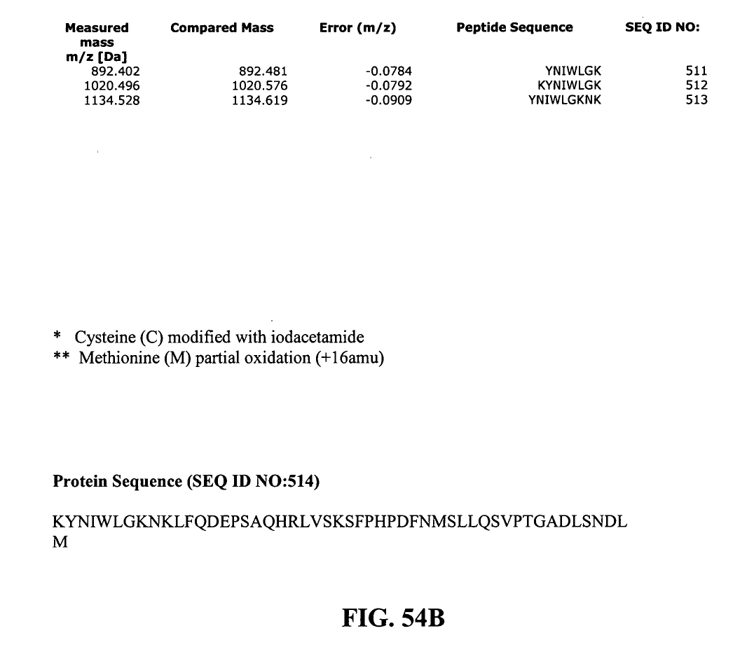

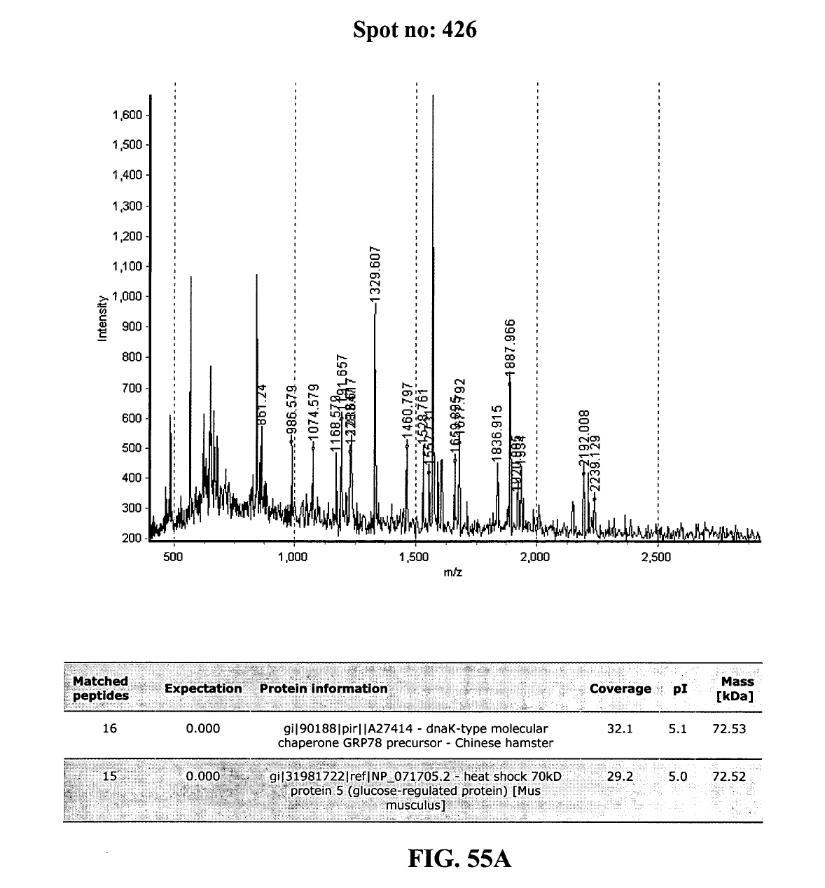

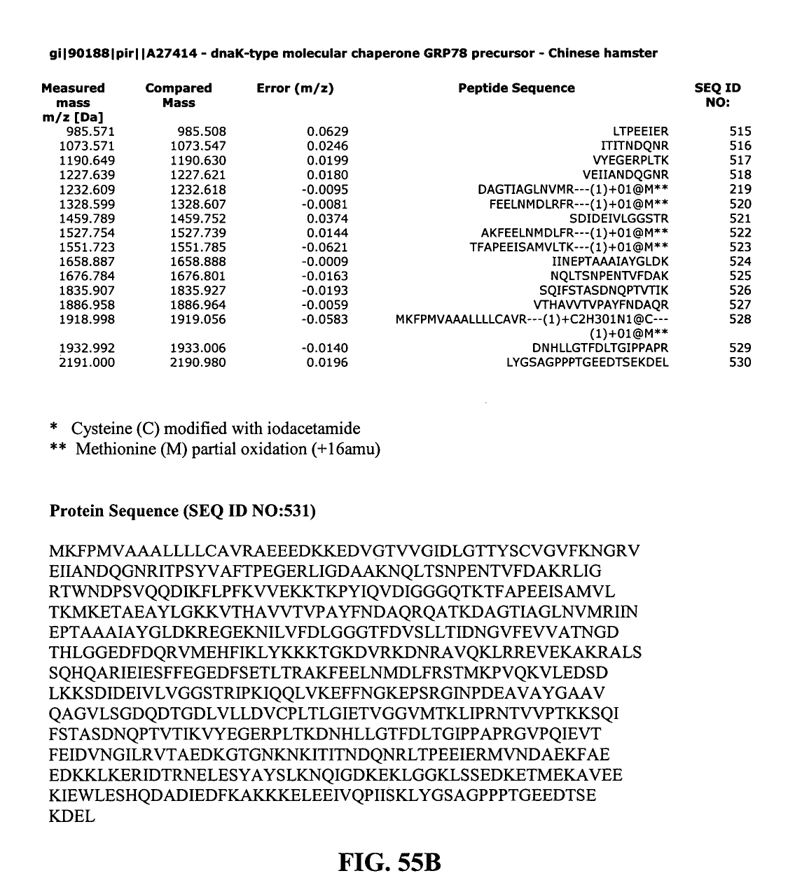

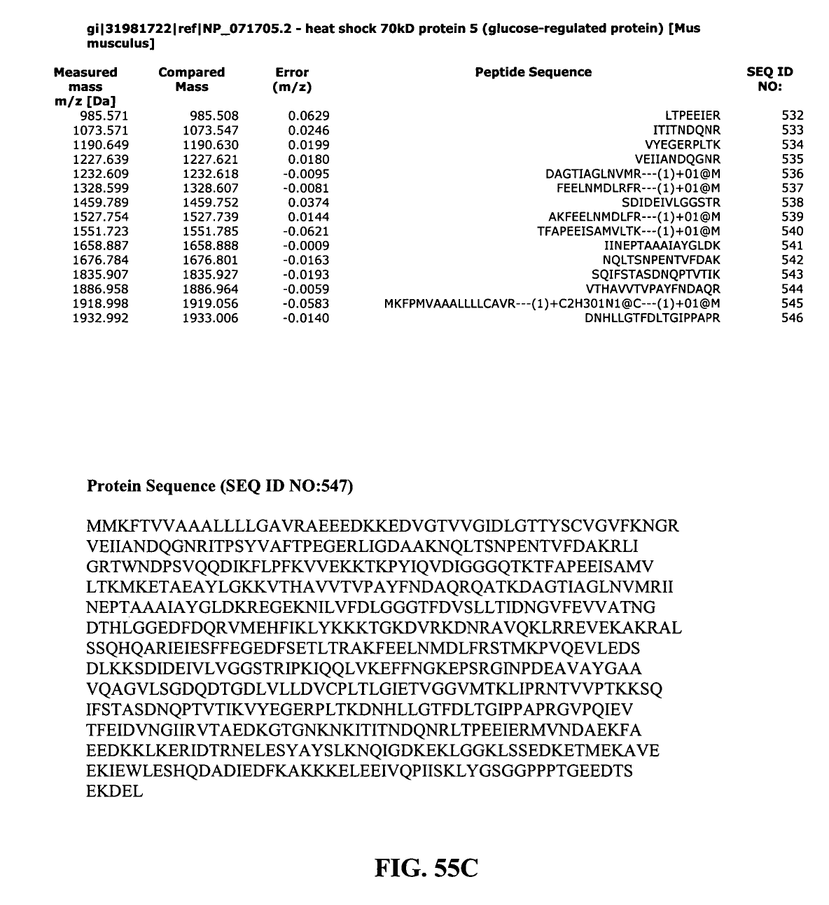

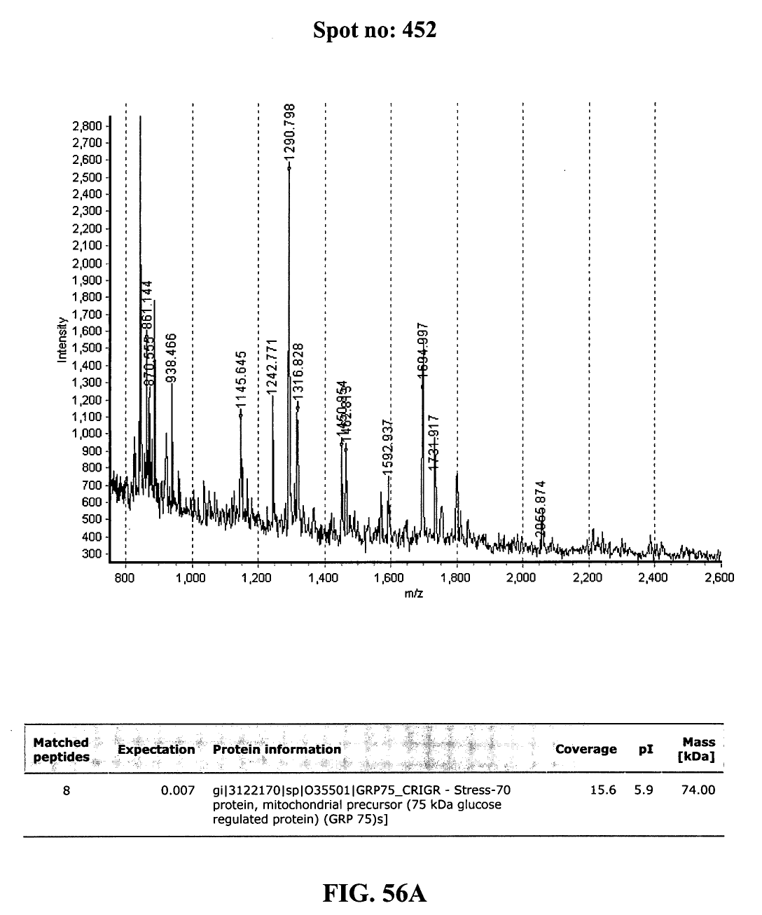

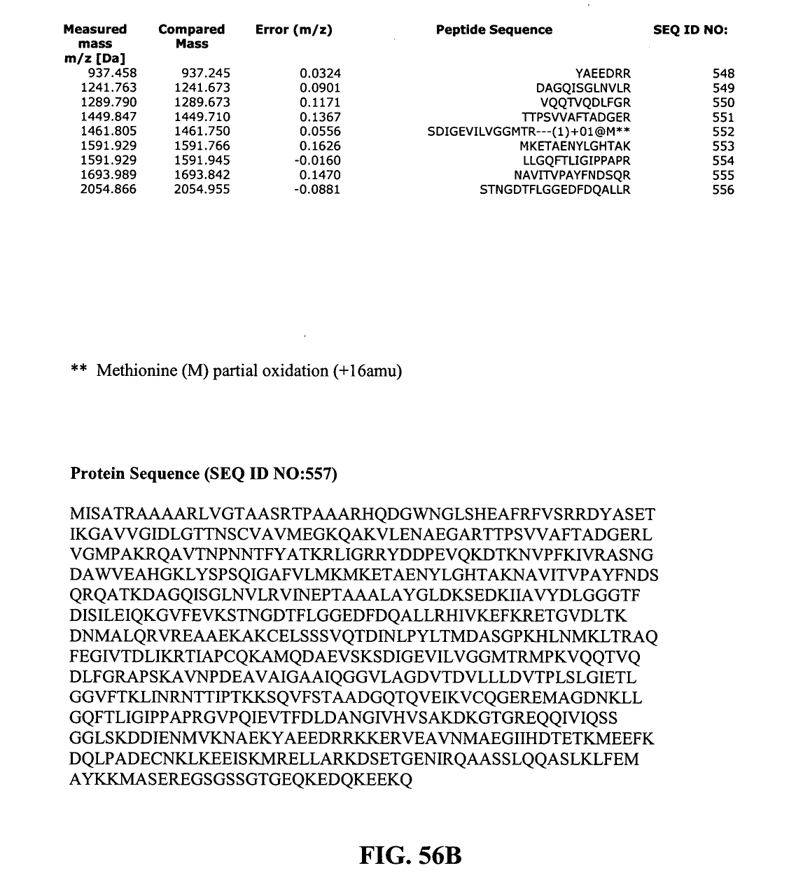

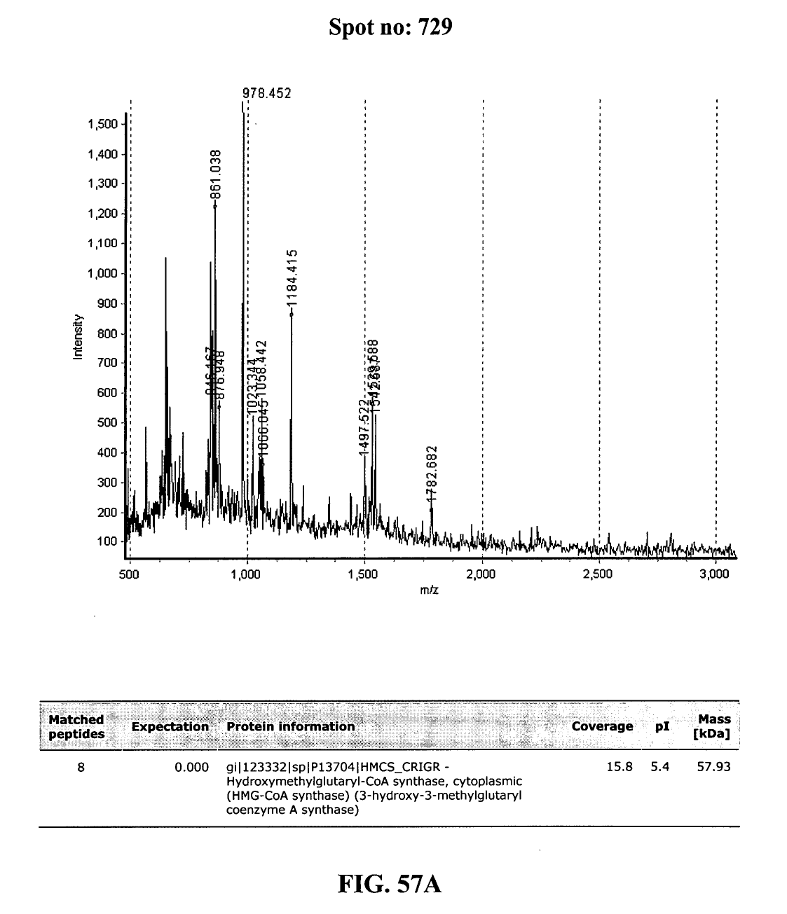

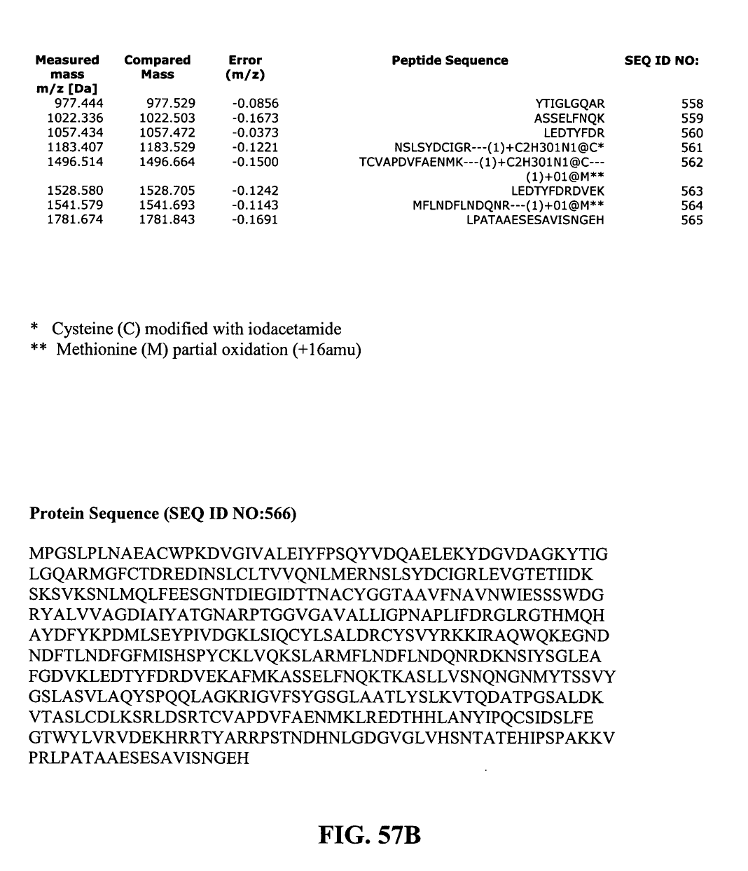

Sequence data for identified proteins are provided in FIGS. 7 through 59. Each figure provides, for a particular protein spot from the DIGE, the spectrum of molecular weights detected in the tryptic digest; the corresponding protein database match or matches, including the number of peptides matched to the predicted tryptic peptides for the protein database entry, the accession number, name, and species of the protein from the database entry, the percent coverage, the isoelectric point and mass; for each molecular weight matched with a predicted mass of a predicted peptide, the measured mass, the predicted (compared) mass, the difference between the two, and the corresponding peptide sequence; and the full length sequence of the protein from the database entry.

Markers for High Cell Growth Rate

The protein expression profile of PA DUKX 378, having a high cell growth rate, was compared to the protein expression profile of PA DUKX 153.8. Tables 4 and 5 list several of the spots identified as differentially expressed in the high maximal cellular productivity cell line. For each of the spots listed in the tables, MALDI sequence analysis identified matches to a corresponding amino acid sequence from Chinese hamsters or from another species. The tables provide, for each spot number, the fold difference in protein levels between the test and control samples, labeled as “Average Ratio”; proteins whose levels are reduced in the test samples are indicated with a negative sign. The tables also provide: the p-value (statistical significance); and the protein name, accession number, and species corresponding to any identified amino acid sequence.

TABLE 4
High Cell Growth Rate Proteins Identified as Novel Homologs of Non-Hamster Proteins

| | | | | | | | | | | No. |
|---|---|---|---|---|---|---|---|---|---|---|
| | Average | | | | | | | | | peptides |
| Decyder | Ratio | | | | | | | | Expectancy | used for |
| Master | (Test/ | | | | Mass Spec | % | | | value - | LC-MS/ |
| no. | Control) | p-value | Accession no. | Protein name | Identification | coverage | pI | Mr | MALDI | MS ID |
| 310 | 1.67 | 5.1 × 10−5 | gi\|17865351 | valosin-containing protein | MALDI ID | 30.3 | 5.1 | 90.02 | 0 | |
| 314 | −1.6 | 3.8 × 10−4 | gi\|600159 | elongation factor 2 | MALDI ID | 23.1 | 6.4 | 96.26 | 0 | |
| 440 | 1.64 | 0.0024 | gi\|38371758 | alpha glucosidase II alpha | MALDI ID | 12.3 | 5.9 | 86.27 | 0 | |
| | | | | subunit isoform 1 | | | | | | |
| 610 | 2.18 | 1.5 × 10−5 | gi\|21411235 | NADH dehydrogenase | MALDI ID | 23.2 | 5.8 | 80.45 | 0 | |
| | | | | (ubiquinone) Fe—S protein 1, | | | | | | |
| | | | | 75 kDa, precursor | | | | | | |
| 624 | 1.54 | 2.7 × 10−4 | gi\|73968066 | PREDICTED: similar to 78 kDa | MALDI ID | 30.3 | 5.1 | 65.46 | 0 | |
| | | | | glucose-regulated protein | | | | | | |
| | | | | precursor (GRP 78) | | | | | | |
| | | | | (Immunoglobulin heavy chain | | | | | | |
| | | | | binding protein) (BiP) | | | | | | |
| | | | | (Endoplasmic reticulum | | | | | | |
| | | | | lumenal Ca(2+) binding | | | | | | |
| | | | | protein grp78) isoform 3 | | | | | | |
| 636 | 1.56 | 3.5 × 10−6 | gi\|73968066 | PREDICTED: similar to 78 kDa | MALDI ID | 26.3 | 5.1 | 65.46 | | |
| | | | | glucose-regulated protein | | | | | | |
| | | | | precursor (GRP 78) | | | | | | |
| | | | | (Immunoglobulin heavy chain | | | | | | |
| | | | | binding protein) (BiP) | | | | | | |
| | | | | (Endoplasmic reticulum | | | | | | |
| | | | | lumenal Ca(2+) binding | | | | | | |
| | | | | protein grp78) isoform 3 | | | | | | |
| 703 | 1.65 | 9.5 × 10−4 | gi\|42542422 | heat shock 70 kDa protein 8 | MALDI ID | 34.8 | 5.3 | 71.08 | 0 | |
| | | | | (Hsc70-ps1) | | | | | | |
| 708 | 1.52 | 0.0035 | gi\|5685 | heat shock 70 kDa protein 8 | MALDI ID | 37.8 | | | 0 | |
| | | | | (Hsc70-ps1) | | | | | | |
| 894 | 1.56 | 0.0016 | gi\|24025637 | heat shock 70 kDa protein 4 | MALDI ID | 9.6 | 5.1 | 94.84 | 0.001 | |
| 990 | 1.61 | 2.7 × 10−10 | gi\|2745838 | Hsp70/Hsp90 organizing | MALDI ID | 27.8 | 6.4 | 63.26 | 0 | |
| | | | | protein | | | | | | |
| 1033 | 1.63 | 2.7 × 10−7 | gi\|16508150 | ERP57 protein (glucose | MALDI ID | 25.7 | 6 | 57.23 | 0 | |
| | | | | regulated protein/protein | | | | | | |
| | | | | disulphide isomerase) | | | | | | |
| 1139 | 1.59 | 3.8 × 10−6 | gi\|52353955 | 3-phosphoglycerate | MALDI ID | 16.9 | 6.1 | 57.37 | 0 | |
| | | | | dehydrogenase | | | | | | |
| 1172 | −1.67 | 3.2 × 10−5 | gi\|73993723 | PREDICTED: similar to | MALDI ID | 19.1 | 5.7 | 53.46 | 0.001 | |
| | | | | Serine/threonine protein | | | | | | |
| | | | | phosphatase 2A, 55 kDa | | | | | | |
| | | | | regulatory subunit B, alpha | | | | | | |
| | | | | isoform (PP2A, subunit B, B- | | | | | | |
| | | | | alpha isoform) (PP2A, subunit | | | | | | |
| | | | | B, B55-alpha isoform) (PP2A, | | | | | | |
| | | | | subunit B, PR55-alpha | | | | | | |
| | | | | isoform) (PP2A, subunit B, | | | | | | |
| | | | | R2-alpha isoform)... isoform 9 | | | | | | |
| 1284 | 1.77 | 0.003 | gi\|21618633 | 3-hydroxy-3-methylglutaryl- | MALDI ID | 21 | 5.6 | 58.16 | 0 | |
| | | | | Coenzyme A synthase 1 | | | | | | |
| 1452 | 1.54 | 0.012 | gi\|51710798 | PREDICTED: similar to | MALDI ID | 8.3 | 4.7 | 50.28 | 0.001 | |
| | | | | tubulin, beta, 2 | | | | | | |
| 1541 | −1.68 | 3.2 × 10−9 | gi\|381964 | actin-related protein | MALDI ID | 39.6 | 6.3 | 42.67 | 0 | |
| 1806 | 1.52 | 2.9 × 10−4 | gi\|433308\| | capping protein alpha | MALDI ID | 20.4 | 5.6 | 32.96 | 0.002 | |
| 1880 | −1.62 | 2.2 × 10−4 | gi\|18026574 | transaldolase | MALDI ID | 25.8 | 7 | 37.54 | 0 | |
| 1959 | −2.23 | 5.3 × 10−8 | gi\|18026574 | transaldolase | MALDI ID | 40.4 | 7 | 37.54 | 0 | |
| 2326 | −1.69 | 6.80E−11 | gi\|73962540 | PREDICTED: similar to | MALDI ID | 30.1 | 5.5 | 27.5 | 0 | |
| | | | | Proteasome activator complex | | | | | | |
| | | | | subunit 2 (Proteasome | | | | | | |
| | | | | activator 28-beta subunit) | | | | | | |
| | | | | (PA28beta) (PA28b) | | | | | | |
| | | | | (Activator of multicatalytic | | | | | | |
| | | | | protease subunit 2) (11S | | | | | | |
| | | | | regulator complex beta | | | | | | |
| | | | | subunit) (REG-beta) isoform 1 | | | | | | |
| 2445 | −1.88 | 1.7 × 10−6 | gi\|61862114 | PREDICTED: similar to SET | MALDI ID | 32.1 | 4.1 | 29.5 | 0.001 | |
| | | | | protein (Phosphatase 2A | | | | | | |
| | | | | inhibitor I2PP2A) (I-2PP2A) | | | | | | |
| | | | | (Template activating factor I) | | | | | | |
| | | | | (TAF-I) (Liver regeneration | | | | | | |
| | | | | related protein LRRGR00002) | | | | | | |
| | | | | (Ab1-115), partial/(SET beta | | | | | | |
| | | | | isoform) | | | | | | |
| 2564 | 1.65 | 2.4 × 10−7 | gi\|14010865 | heat shock 27 kDa protein 1 | MALDI ID | 34.1 | 6.1 | 22.86 | 0 | |
| 2733 | 1.52 | 7.1 × 10−9 | gi\|13386316 | actin related protein M2 | MALDI ID | 12.7 | 5 | 42.05 | 0.002 | |
| 3495 | 1.5 | 1.90E−06 | gi\|6755911\| | Thioredoxin 1 | LC-MS/MS ID | 31.43 | 4.8 | 11.68 | | 3 |
| | | | gi\|549078\| | | | | | | | |
| | | | sp\|P10639\| | | | | | | | |
| 3498 | 1.5 | 5.10E−05 | gi\|12230575\| | SH3 domain-binding glutamic | LC-MS/MS ID | 34.21 | 4.87 | 12.81 | | 4 |
| | | | sp\|O75368\| | acid-rich-like protein | | | | | | |

TABLE 5
High Cell Growth Rate Known Hamster Proteins

| | Average Ratio | | | | |
|---|---|---|---|---|---|
| Spot no. | (Test/Control) | p-value | gi accession no. | Protein name | Species |
| 314 | −1.6 | 3.8 × 10−4 | gi\|600159 | elongation factor 2 | Cricetulus |
| | | | | | griseus |
| 990 | 1.61 | 2.7 × 10−10 | gi\|2745838 | Hsp70/Hsp90 organizing protein | Cricetulus |
| | | | | | griseus |
| 1033 | 1.63 | 2.7 × 10−7 | gi\|16508150 | ERP57 protein (glucose regulated protein/ | Cricetulus |
| | | | | protein disulphide isomerase) | griseus |
| 1880 | −1.62 | 2.2 × 10−4 | gi\|18026574 | transaldolase | Cricetulus |
| | | | | | griseus |
| 1959 | −2.23 | 5.3 × 10−8 | gi\|18026574 | transaldolase | Cricetulus |
| | | | | | griseus |

TABLE 6
HCGR3 List

| | Average | | | | | % | | | | |
|---|---|---|---|---|---|---|---|---|---|---|
| | Ratio | | | | | cov- | | | Expectancy | No. peptides |
| Spot | (Test/ | | | | Mass Spec | er- | | | value - | used for LC- |
| no. | Control) | p-value | Accession no. | Protein name | Identification | age | pI | Mr | MALDI | MS/MS ID |
| 428 | 3.57 | 1.70E−08 | gi\|600159 | elongation factor 2 | MALDI ID | 29.8 | 6.4 | 96.26 | 0 | |
| 447 | 3.67 | 4.10E−08 | gi\|600159 | elongation factor 2 | MALDI ID | 28.3 | 6.4 | 96.26 | 0 | |
| 705 | 1.84 | 3.6 × 10−15 | gi\|21704020 | NADH dehydrogenase | MALDI ID | 17.2 | 5.5 | 80.76 | 0 | |
| | | | | (ubiquinone) Fe—S protein 1 | | | | | | |
| 716 | 2.32 | 5.90E−06 | gi\|73968066 | PREDICTED: similar to 78 kDa | MALDI ID | 30 | 5.1 | 65.46 | 0 | |
| | | | | glucose-regulated | | | | | | |
| | | | | protein precursor (GRP 78) | | | | | | |
| | | | | (Immunoglobulin heavy | | | | | | |
| | | | | chain binding protein) | | | | | | |
| | | | | (BiP) (Endoplasmic | | | | | | |
| | | | | reticulum lumenal Ca(2+) | | | | | | |
| | | | | binding protein grp78) | | | | | | |
| | | | | isoform 3 | | | | | | |
| 730 | 1.76 | 0.024 | gi\|73968066 | PREDICTED: similar to 78 kDa | MALDI ID | 26.1 | 5.1 | 65.46 | 0 | |
| | | | | glucose-regulated | | | | | | |
| | | | | protein precursor (GRP 78) | | | | | | |
| | | | | (Immunoglobulin heavy | | | | | | |
| | | | | chain binding protein) | | | | | | |
| | | | | (BiP) (Endoplasmic | | | | | | |
| | | | | reticulum lumenal Ca(2+) | | | | | | |
| | | | | binding protein grp78) | | | | | | |
| | | | | isoform 3 | | | | | | |
| 797 | −1.87 | 5.8 × 10−15 | gi\|1339938 | glycerol-3-phosphate | MALDI ID | 14.3 | 6.2 | 81.49 | 0.002 | |
| | | | | dehydrogenase | | | | | | |
| 999 | −1.58 | 1.6 × 10−14 | gi\|34785817 | Copine I | MALDI ID | 10.6 | 5.4 | 59.59 | 0.003 | |
| 1017 | −1.7 | 1.8 × 10−15 | gi\|34853001 | similar to UDP-N- | MALDI ID | 11.6 | 5.4 | 57.16 | 0.005 | |
| | | | | acteylglucosamine | | | | | | |
| | | | | pyrophosphorylase 1-like 1 | | | | | | |
| 1039 | 1.61 | 7.50E−15 | gi\|31981679 | heat shock protein 1 | MALDI ID | 41.2 | 5.7 | 61.11 | 0 | |
| | | | | (chaperonin) | | | | | | |
| 1069 | −1.64 | 3.1 × 10−9 | gi\|27762594 | alpha tubulin | MALDI ID | 21.6 | 4.9 | 50.69 | 0 | |
| 1137 | 1.58 | 9.9 × 10−14 | gi\|22324680 | FK506 binding protein 4 | MALDI ID | 31.3 | 5.7 | 45.87 | 0.001 | |
| 1180 | 1.54 | 3.9 × 10−14 | gi\|48675860 | eukaryotic translation | MALDI ID | 13.1 | 6.5 | 51.1 | 0.005 | |
| | | | | initiation factor 2B, subunit | | | | | | |
| | | | | 3 gamma | | | | | | |
| 1263 | 1.61 | 3.4 × 10−11 | gi\|3121992 | Aldehyde dehydrogenase, | MALDI ID | 37 | 5.8 | 54.83 | 0 | |
| | | | | mitochondrial (ALDH | | | | | | |
| | | | | class 2) (ALDH1) (ALDH- | | | | | | |
| | | | | E2) | | | | | | |
| 1303 | −1.62 | 1.3 × 10−13 | gi\|25742757 | glutathione synthetase | MALDI ID | 16.2 | 5.5 | 52.61 | 0 | |
| 1336 | −1.64 | 3 × 10−10 | gi\|62296810 | Protein disulfide-isomerase | MALDI ID | 15.2 | 5 | 48.55 | 0 | |
| | | | | A6 precursor (Protein | | | | | | |
| | | | | disulfide isomerase P5) | | | | | | |
| | | | | (Calcium-binding protein | | | | | | |
| | | | | 1) (CaBP1) | | | | | | |
| 1344 | 1.9 | 4.9 × 10−15 | gi\|48146175 | EIF3S6 (eukaryotic | MALDI ID | 30.8 | 5.7 | 52.59 | 0 | |
| | | | | initiation factor 3, subunit | | | | | | |
| | | | | 6) | | | | | | |
| 1376 | 1.92 | 1.7 × 10−12 | gi\|74007151 | PREDICTED: similar to | MALDI ID | 32.3 | 9.4 | 44.26 | 0.009 | |
| | | | | Alpha enolase (2-phospho- | | | | | | |
| | | | | D-glycerate hydro-lyase) | | | | | | |
| | | | | (Non-neural enolase) | | | | | | |
| | | | | (NNE) (Enolase 1) | | | | | | |
| | | | | (Phosphopyruvate | | | | | | |
| | | | | hydratase) (C-myc | | | | | | |
| | | | | promoter-binding protein) | | | | | | |
| | | | | (MBP-1) (MPB-1) | | | | | | |
| | | | | (Plasminogen-binding | | | | | | |
| | | | | protein) | | | | | | |
| 1393 | 1.54 | 9.6 × 10−12 | gi\|74007151 | PREDICTED: similar to | MALDI ID | 32.5 | 9.4 | 44.26 | 0 | |
| | | | | Alpha enolase (2-phospho- | | | | | | |
| | | | | D-glycerate hydro-lyase) | | | | | | |
| | | | | (Non-neural enolase) | | | | | | |
| | | | | (NNE) (Enolase 1) | | | | | | |
| | | | | (Phosphopyruvate | | | | | | |
| | | | | hydratase) (C-myc | | | | | | |
| | | | | promoter-binding protein) | | | | | | |
| | | | | (MBP-1) (MPB-1) | | | | | | |
| | | | | (Plasminogen-binding | | | | | | |
| | | | | protein) | | | | | | |
| 1403 | −1.54 | 1.70E−09 | gi\|73979721 | PREDICTED: similar to | MALDI ID | 20.6 | 5.3 | 45.73 | 0.003 | |
| | | | | Ribonucleoside- | | | | | | |
| | | | | diphosphate reductase M2 | | | | | | |
| | | | | chain (Ribonucleotide | | | | | | |
| | | | | reductase small chain) | | | | | | |
| | | | | isoform 1 | | | | | | |
| 1438 | −1.6 | 1.3 × 10−10 | gi\|38197664 | Adhesion regulating | MALDI ID | 15 | 4.9 | 42.43 | 0.004 | |
| | | | | molecule 1 | | | | | | |
| 1470 | −1.82 | 1.00E−10 | gi\|70909332 | hypoxia-inducible factor 1, | MALDI ID | 14.9 | 5.6 | 40.39 | 0.008 | |
| | | | | alpha subunit inhibitor | | | | | | |
| 1526 | −1.59 | 8.9 × 10−16 | gi\|1351867 | Actin, cytoplasmic 1 (Beta- | MALDI ID | 34.9 | 5.2 | 42.06 | 0 | |
| | | | sp\|P48975\| | actin) | | | | | | |
| 1706 | −1.82 | 3.10E−13 | gi\|16758446 | isocitrate dehydrogenase 3 | MALDI ID | 24 | 6.5 | 40.05 | 0.001 | |
| | | | | (NAD+) alpha | | | | | | |
| 1738 | −1.61 | 1.3 × 10−15 | gi\|73951310 | PREDICTED: similar to | MALDI ID | 33.2 | 5.9 | 35.1 | 0.008 | |
| | | | | isocitrate dehydrogenase 3 | | | | | | |
| | | | | (NAD+) alpha isoform 2 | | | | | | |
| 1774 | −1.7 | 6.7 × 10−16 | gi\|2114406 | aldo-keto reductase | MALDI ID | 23.1 | 6.2 | 36.61 | 0.002 | |
| 1815 | −1.51 | 1.2 × 10−8 | gi\|1407651 | Lasp-1 | MALDI ID | 41 | 5.1 | 23.08 | 0 | |
| 1916 | −1.68 | 1.6 × 10−15 | gi\|17391477 | Annexin 5 | MALDI ID | 38.8 | 4.9 | 35.96 | 0 | |
| 2328 | −1.67 | 0 | gi\|17939632 | Platelet-activating factor | MALDI ID | 17.5 | 5.6 | 25.72 | 0.003 | |
| | | | | acetylhydrolase, isoform | | | | | | |
| | | | | Ib, beta subunit 30 kDa | | | | | | |
| 2353 | −1.6 | 4.8 × 10−10 | gi\|68085578 | Tyrosine 3/tryptophan 5 - | MALDI ID | 44.5 | 4.7 | 27.87 | 0 | |
| | | | | monooxygenase activation | | | | | | |
| | | | | protein, zeta polypeptide | | | | | | |
| 2371 | 1.55 | 6 × 10−12 | gi\|17389815 | Triosephosphate isomerase 1 | MALDI ID | 22.1 | 6.4 | 26.91 | 0.001 | |
| 3034 | −1.69 | 3.70E−11 | gi\|124231\| | Eukaryotic translation | LC-MS/MS ID | 20.13 | 5.07 | 16.82 | | 4 |
| | | | sp\|P10160\| | initiation factor 5A (eIF- | | | | | | |
| | | | | 5A) (eIF-4D) | | | | | | |
| 3050 | 1.58 | 1.50E−09 | gi\|9910216 AND | Prefoldin subunit 5 | LC-MS/MS ID | 16.49 | 6.83 | 21.64 | | 3 |
| | | | gi\|73921733\| | | | | | | | |
| | | | sp\|Q5RAY0\| | | | | | | | |
| 3244 | 1.79 | 2.5 × 10−14 | gi\|14625464 | stathmin | MALDI ID | 36.9 | 5.9 | 17.2 | 0.007 | 3 |
| 3892 | 1.56 | 8.30E−10 | gi\|2842685\| | Myotrophin (V-1 protein) | LC-MS/MS ID | 18.64 | 5.11 | 12.87 | | 2 |
| | | | sp\|Q91955 | (Granule cell | | | | | | |
| | | | | differentiation protein) | | | | | | |

Statistics used in Decyder analysis, +/−1.5 fold change, t-test < 0.05

Sequence data for identified proteins are provided in FIGS. 60 through 112. Each figure provides, for a particular protein spot from the DIGE, the spectrum of molecular weights detected in the tryptic digest; the corresponding protein database match or matches, including the number of peptides matched to the predicted tryptic peptides for the protein database entry, the accession number, name, and species of the protein from the database entry, the percent coverage, the isoelectric point and mass; for each molecular weight matched with a predicted mass of a predicted peptide, the measured mass, the predicted (compared) mass, the difference between the two, and the corresponding peptide sequence; and the full length sequence of the protein from the database entry.

### Example 4

### Proteins Differentially Expressed in Cells with Sustained High Cell Viability or High Peak Cell Density

Table 7 lists several of the spots identified as differentially expressed in the cells with sustained high cell viability using methods as described in Example 3. Sequence data for the identified proteins are provided in FIGS. 113 through 127. Table 8 lists several of the spots identified as differentially expressed in the cells with high peak cell density using similar methods; corresponding sequence data are shown in FIGS. 128 through 138. The tables provide, for each spot number, the fold difference in protein levels between the test and control samples, labeled as “Average Ratio”; proteins whose levels are reduced in the test samples are indicated with a negative sign. The tables also provide the p-value that the differences in expression would be the result of random chance and the protein name and accession number corresponding to any identified amino acid sequence. The resulting peptides were analyzed by mass spectrometry. MALDI is used, particularly for highly abundant samples on gels, for peptide mass fingerprinting. For lower abundance samples, LC-MS/MS using an MDLC LTQ machine is used. In the MALDI sequence analysis, the molecular weights of the trypsin fragments were compared to predicted molecular weights of trypsin fragments of known sequences. In the tables, “% coverage” refers to the percentage of the total length of a database sequence for which corresponding trypsin fragments were detected in the experiment. pI and M_{R} refer to the apparent isoelectric point and apparent molecular weight of the protein spot.

TABLE 7
Differentially expressed proteins in cells with sustained high cell viability
(Statistics used in Decyder analysis, +/−1.5 fold change, t-test < 0.05)

| | | | | | | | | | | No. |
|---|---|---|---|---|---|---|---|---|---|---|
| | | | | | | | | | | peptides |
| | Average | | | | | | | | | used for |
| | Ratio | | | | | | | | Expectancy | LC- |
| Spot | (Test/ | | | | Mass Spec | | | | value - | MS/MS |
| no. | Control) | p-value | Accession no. | Protein name | Identification | % coverage | pI | Mr | MALDI | ID |
| 539 | 1.93 | 1.90E−05 | gi\|38969850 | Chaperonin containing TCP1, | MALDI ID | 30.3 | 6.2 | 61.2 | 0 | |
| | | | | subunit 3 (gamma) | | | | | | |
| 559 | −1.53 | 0.03 | gi\|15030102 | Sdha protein (succinate | MALDI ID | 23.1 | 7.2 | 73.39 | 0 | |
| | | | | dehydrogenase) | | | | | | |
| 800 | 2.89 | 2.90E−10 | gi\|21618633 | 3-hydroxy-3 methylglutaryl- | MALDI ID | 13.3 | 5.6 | 58.16 | 0.005 | |
| | | | | Coenzyme A synthase 1 | | | | | | |
| 870 | −1.84 | 0.014 | gi\|23272966\| | Atp5b protein | MALDI ID | 21.4 | 5.2 | 56.65 | 0 | |
| 915 | −1.54 | 0.04 | gi\|3121992\| | Aldehyde dehydrogenase, | MALDI ID | 35.6 | 5.8 | 54.83 | 0 | |
| | | | sp\|P81178\| | mitochondrial (ALDH class 2) | | | | | | |
| 1393 | −1.61 | 0.0082 | gi\|18026574 | transaldolase | MALDI ID | 29.4 | 7 | 37.54 | 0 | |
| | | | gi\|46396972\| | | | | | | | |
| | | | sp\|Q8VI73\| | | | | | | | |
| 2022 | 1.53 | 0.00054 | gi\|47496673 | GRP2 (Growth factor receptor- | MALDI ID | 52.5 | 6.1 | 25.24 | 0 | |
| | | | | bound protein 2) | | | | | | |
| 2050 | −1.58 | 0.023 | gi\|34879492 | PREDICTED: similar to tumor | MALDI ID | 18.2 | 5.2 | 19.09 | 0.009 | |
| | | | | protein, translationally- | | | | | | |
| | | | | controlled 1 | | | | | | |
| 2250 | −1.63 | 0.01 | gi\|1313936 | mitochondrial ribosomal protein | MALDI ID | 22.7 | 9.4 | 21.69 | 0.005 | |
| 2459 | 2.08 | 0.0021 | gi\|3212116 | prefoldin subunit 2 | MALDI ID | 40.9 | 6.8 | 16.82 | 0 | |
| 2543 | 1.72 | 2.80E−05 | gi\|14625464 | stathmin | MALDI ID | 53.7 | 5.9 | 17.2 | 0 | |
| 2863 | 1.72 | 0.013 | gi\|9963901 | profilin II | MALDI ID | 29.5 | 6.6 | 16.05 | 0 | |
| 2897 | 1.77 | 0.0013 | gi\|73961217 | PREDICTED: similar to ATP | MALDI ID | 31.4 | 6.1 | 12.82 | 0.003 | |
| | | | | synthase alpha chain, | | | | | | |
| | | | | mitochondrial precursor | | | | | | |
| | | | | isoform 3 | | | | | | |
| 3340 | −7.54 | 7.90E−13 | gi\|2493416\| | S100 calcium-binding protein | LC-MS/MS ID | 17.35 | 5.9 | 11.47 | | 2 |
| | | | sp\|Q99584 | A13 | | | | | | |
| 3349 | −1.55 | 0.00011 | gi\|50401358\| | Calpactin I light chain (S100 | LC-MS/MS ID | 20.62 | 6.82 | 11.2 | | 2 |
| | | | sp\|P62504\| | calcium-binding protein A10) | | | | | | |

TABLE 8
HCD3 Protein List
All test samples vs all control samples (day 3, 5, 7), (Filters - 1.2 fold up/down regulation,
t-test < 0.01, 2-way anova < 0.01, Decyder statistical analysis)

| | Fold | | | | | | | | No. |
|---|---|---|---|---|---|---|---|---|---|
| | Change | | | | | | | | peptides |
| | (All | | | | | | | Expectancy | used for |
| Spot | Test/All | Accession | | Mass Spec | % | | | value - | LC-MS/MS |
| no. | Control) | number | Protein Name | Identification | coverage | pI | Mr | MALDI | ID |
| 619 | −1.34 | gi\|20532062\| | Dipeptidyl-peptidase 3 | LC-MS/MS ID | 8.4 | 5.22 | 82.29 | | 7 |
| | | sp\|Q99KK7\| | (Dipeptidyl-peptidase III) | | | | | | |
| 984 | 1.2 | gi\|75773247\| | Chaperonin subunit 6a | MALDI ID | 20.3 | 6.6 | 58.46 | 0.007 | |
| | | | (zeta) | | | | | | |
| 1078 | 1.2 | gi\|179102\| | aspartyl-tRNA synthetase | MALDI ID | 16.4 | 6.2 | 57.57 | 0.005 | |
| 1148 | −1.23 | gi\|135446\| | Tubulin beta-1 chain | LC-MS/MS ID | 6.52 | 4.78 | 49.91 | | 3 |
| | | sp\|P09203\| | (Beta-tubulin class-I) | | | | | | |
| 1154 | −1.25 | gi\|1353212\| | Vimentin | LC-MS/MS ID | 4.69 | 4.94 | 51.85 | | 2 |
| | | sp\|P48670\| | | | | | | | |
| 1157 | 1.3 | gi\|62511005\| | D-3-phosphoglycerate | LC-MS/MS ID | 10.88 | 6.48 | 56.55 | | 5 |
| | | sp\|Q60HD7\| | dehydrogenase | | | | | | |
| 1373 | −1.24 | gi\|729443\| | Protein disulfide- | LC-MS/MS ID | 20.73 | 5.04 | 48.16 | | 8 |
| | | sp\|P38660\| | isomerase A6 precursor | | | | | | |
| 1382 | −1.24 | gi\|729443\| | Protein disulfide- | LC-MS/MS ID | | 5.04 | 48.16 | | |
| | | sp\|P38660\| | isomerase A6 precursor | | | | | | |
| 1500 | −1.23 | gi\|548710\|sp\|Q05186\| | Reticulocalbin-1 | LC-MS/MS ID | 15.08 | 4.7 | 38.11 | | 7 |
| | | | precursor | | | | | | |
| 1656 | 1.24 | gi\|729023\| | Macrophage capping | LC-MS/MS ID | 23.86 | 6.73 | 39.24 | | 6 |
| | | sp\|P24452 | protein (Myc basic motif | | | | | | |
| | | | homolog 1) | | | | | | |
| 1682 | 1.32 | gi\|21466051 | Chain A, Crystal | MALDI ID | 20.9 | 6.1 | 34.95 | 0 | |
| | | | Structure Of The | | | | | | |
| | | | Mitochondrial Serine | | | | | | |
| | | | Protease Htra2 | | | | | | |
| 2324 | −1.31 | gi\|61227509\| | Latexin (Endogenous | LC-MS/MS ID | 14.8 | 5.77 | 25.58 | | 3 |
| | | sp\|Q64361\| | carboxypeptidase | | | | | | |
| | | | inhibitor) | | | | | | |

### Example 5

### mRNA Expression Profiling

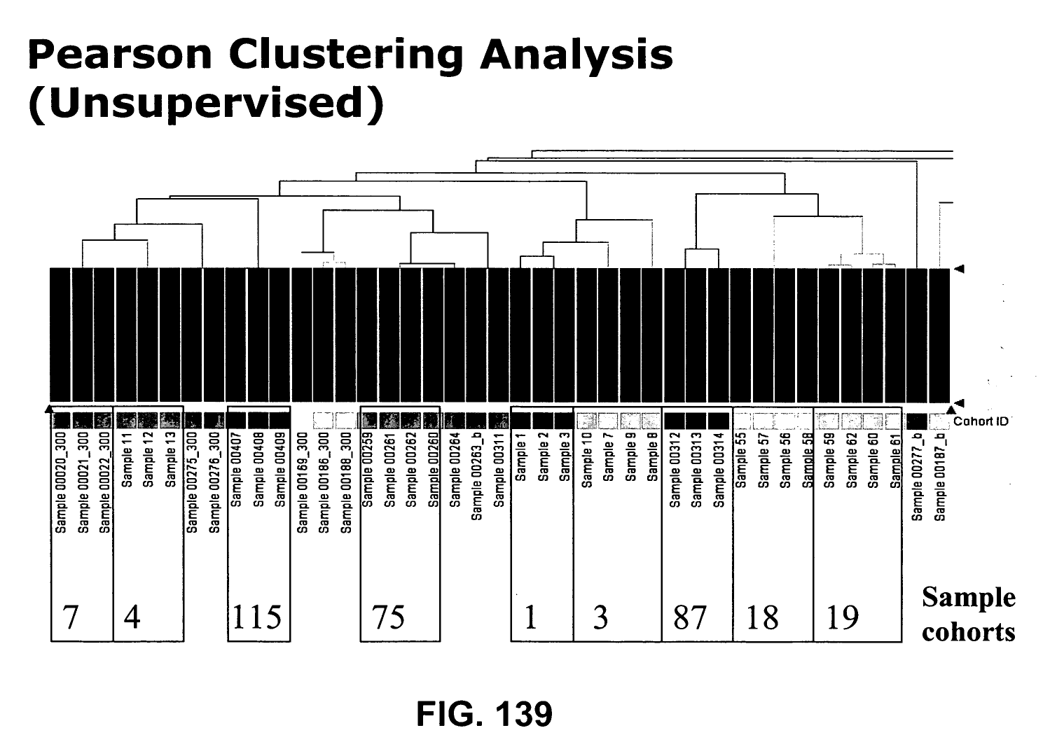

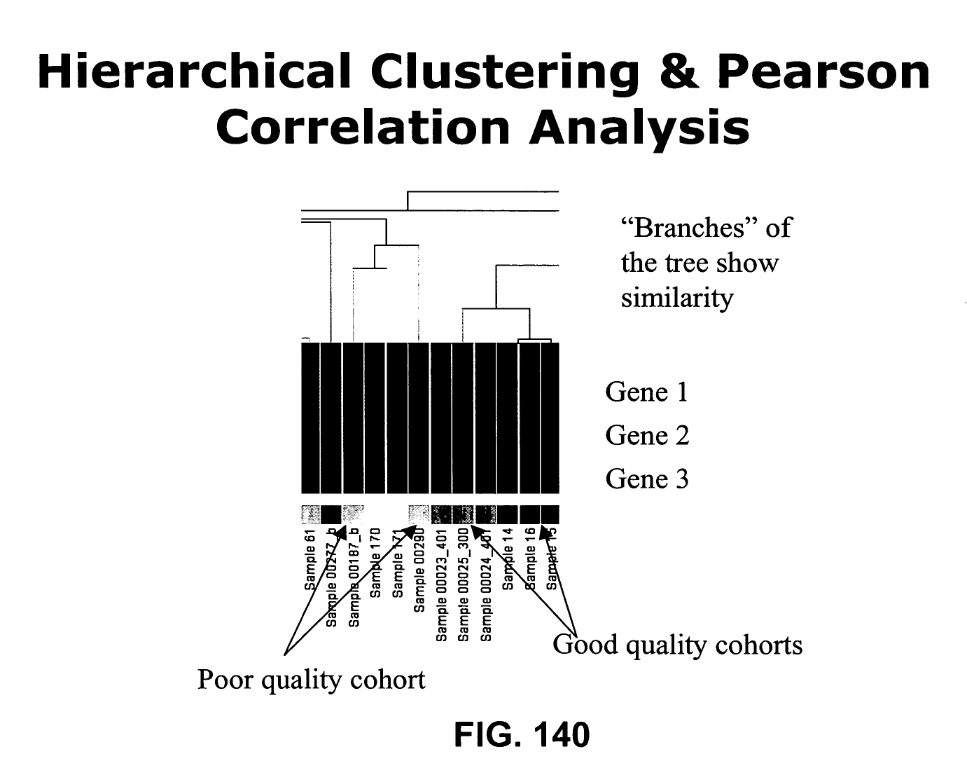

RNA samples from test and control CHO cell lines were obtained and analyzed on a microchip containing probes for CHO mRNA sequences as described in U.S. Patent Application Publication US2006/0010513, the complete contents of which are herein incorporated by reference. The hybridization cocktail was spiked with a fragmented cRNA standard to generate a standard curve using labeled, fragmented cRNA of control sequences at known concentrations, permitting normalization of the data and assessment of chip sensitivity and saturation. The scan data were quality controlled using the 3′/5′ ratio of β-actin and GAPDH, the signal intensity and consistency, and the percent present. Generally, data normalization was performed using software tools Affy 5.0 and Genesis 2.0; or dChiP (see Li et al. (2001) Proc. Natl. Acad. Sci. USA 98:31-36 and Li et al. (2001) Genome Biol. 2:0032.1-0032.11) and Genespring. A P_{Value} less than or equal to 0.05 and a fold-change minimum between the test and control lines of 1.2 was required before a gene would be further considered. An unsupervised Pearson Clustering Analysis is depicted in FIGS. 139 and 140.

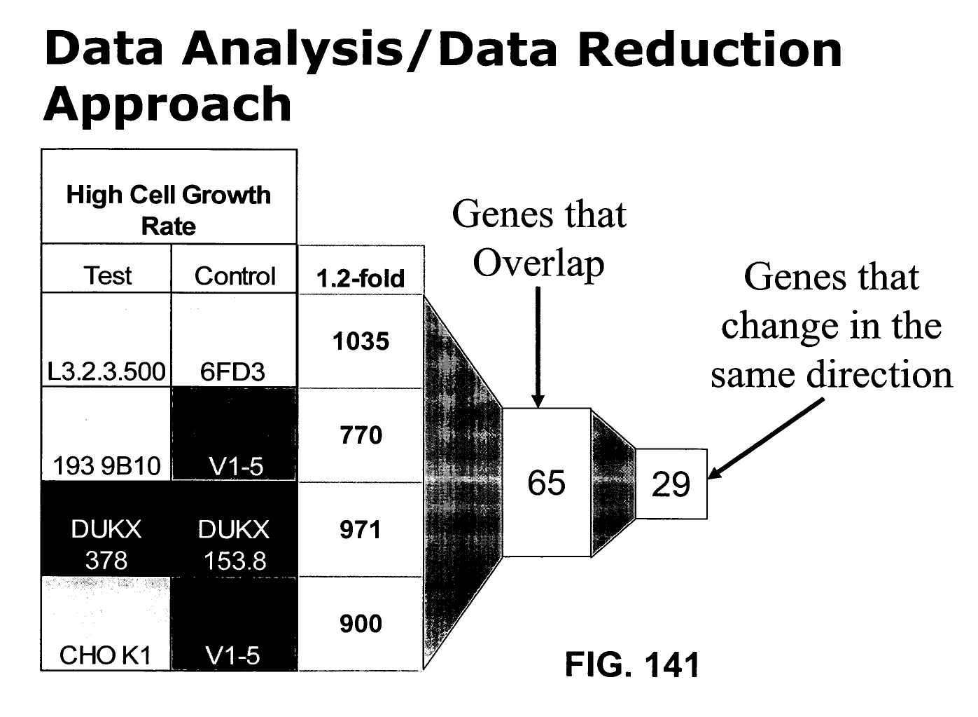

An exemplary method of data analysis is depicted in FIG. 141. Pairs of test and control cell lines for the high cell growth rate were compared and mRNA expression patterns meeting the 1.2-fold difference requirement were identified. Of those, the 65 genes that were differentially expressed in each of four different pairs of test and control cell lines were identified. Of the 65, 29 were either consistently up-regulated or consistently down-regulated in the test cell lines; these were given a higher priority for further analysis.

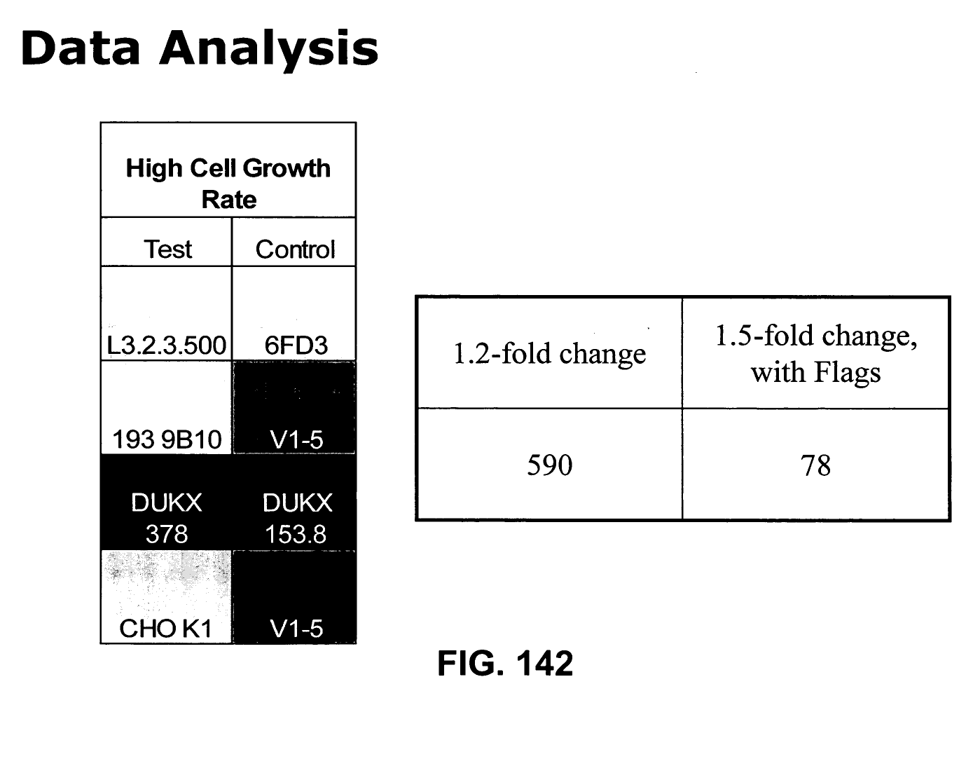

An exemplary method of data analysis that does not rely on pairwise differences is depicted in FIG. 142. 590 genes were identified whose average expression levels in the high cell growth rate test CHO cell lines as a group were at least 1.2-fold higher than the average expression in the group of control CHO cell lines. When a 1.5-fold difference in expression was required and additional, more stringent statistical analysis was applied, 78 genes passed the criteria; these were given a higher priority for further analysis.

### Example 6

### Genes Differentially Expressed in Cells with High Maximum Cellular Productivity

A summary of nucleic acids identified as differentially expressed in cells with high maximum cellular productivity is provided in Tables 9 and 10. For each nucleic acid, a qualifier name, symbol, and title are provided, as well as whether the nucleic acid is up-regulated or down-regulated in the cells with higher maximum cellular productivity. For nucleic acids with human or mouse homologs in the Unigene database, the table provides Unigene ID numbers and statistics relating to the comparison, including e-values, percent sequence identities between the CHO sequence and the Unigene databank entries, and percent coverage (“% QC”).

Nucleic acids encoding proteins associated with the endoplasmic reticulum (ER) or the Golgi complex may contribute to cellular productivity, particularly for the production of a secreted protein. Table 11 summarizes nucleic acids that are differentially expressed by a factor of at least 1.2 in cells overexpressing PACE and encode an ER-associated protein. Table 12 summarizes nucleic acids that are differentially expressed by a factor of at least 1.2 in cells overexpressing PACE and encode a Golgi-associated protein.

TABLE 9
High Max Qp NA Unknown CHO Sequences

| | | | Human | | |
|---|---|---|---|---|---|
| Qualifier List | Symbol | Title | Unigene ID | eValue | % ID |
| WAN0088NU_at | LMNA | Lamin A/C | Hs.491359 | 3E−23 | 85.7143 |
| (SEQ ID NO: 1478) | | | | | |
| WAN0088OT_at | NA | WAN0088OT 10595D-F11 | #N/A | | |
| (SEQ ID NO: 1479) | | | | | |
| WAN0088PY_at | DDX5 | DEAD (Asp-Glu_Ala-Asp) box | Hs.416922 | 0.001 | 91.6667 |
| (SEQ ID NO: 1480) | | polypeptide 5 | | | |
| WAN0088T3_at | ANXA1 | Annexin A1 | Hs.494173 | 6E−76 | 87.372 |
| (SEQ ID NO: 1481) | | | | | |
| WAN0088ZP_at | PAWR | PRKC, apoptosis, WT1, regulator | Hs.406074 | 4E−10 | 91.5254 |
| (SEQ ID NO: 1482) | | | | | |
| WAN00895Y_f_at | DQ390542.2 | Mitochondrial cytochrome b | #N/A | | |
| (SEQ ID NO: 1483) | | | | | |
| WAN008C42_at | CD36 | CD36 antigen | Hs.120949 | 1.00E−05 | 90.9091 |
| (SEQ ID NO: 1484) | | | | | |
| WAN008CJ1_at | ERP70 | Protein disulfide isomerase- | Hs.93659 | 1E−120 | 89.6277 |
| (SEQ ID NO: 1485) | | associated 4 | | | |
| WAN008DT7_at | GSTO1 | Glutathione S-transferase omega 1 | Hs.190028 | 5E−65 | 83.9437 |
| (SEQ ID NO: 1486) | | | | | |
| WAN008EA0_at | VCP | Valosin-containing protein | Hs.529782 | 0 | 90.7273 |
| (SEQ ID NO: 1487) | | | | | |
| WAN008F1I_at | SHOC2 | Soc-2 suppressor of clear homolog | Hs.104315 | 2E−18 | 85.0575 |
| (SEQ ID NO: 1488) | | (C. elegans) | | | |
| WAN008F2S_at | NA | WAN008F2S 11165A-F02 | #N/A | | |
| (SEQ ID NO: 1489) | | | | | |
| WAN013HX8_x_at | EIF4A2 | Eukaryotic translation initiation | Hs.478553 | 0 | 90.824 |
| (SEQ ID NO: 1490) | | factor 4A, isoform 2 | | | |
| WAN013HXR_x_at | STAT6 | signal transducer and activator of | #N/A | | |
| (SEQ ID NO: 1491) | | transcription 6 | | | |
| WAN013I1P_at | HNRPA2B1 | Heterogeneous nuclear | Hs.487774 | 0 | 97.2222 |
| (SEQ ID NO: 1492) | | ribonucleoprotein A2/B1 | | | |
| WAN013I1T_x_at | DQ390542.2 | Mitochondrial NADH | #N/A | | |
| (SEQ ID NO: 1493) | | dehydrogenase subunit 2 | | | |
| WAN013I66_f_at | Vim | Vimentin (Vim), mRNA | Hs.533317 | 1E−126 | 92.2559 |
| (SEQ ID NO: 1494) | | | | | |
| WAN013IA0_at | HPRT1 | hypoxanthine | Hs.412707 | 2.00E−66 | 83.0986 |
| (SEQ ID NO: 1495) | | phosphoribosyltransferase 1 | | | |
| | | (Lesch-Nylan syndrome) | | | |
| WAN013IAB_x_at | TP53 | Tumor protein p53 (Li-Fraumeni | Hs.408312 | 1E−150 | 82.4477 |
| (SEQ ID NO: 1496) | | syndrome) | | | |

| | | Mouse | | | | |
|---|---|---|---|---|---|---|
| Qualifier List | % QC | Unigene ID | eValue | % ID | % QC | |
| WAN0088NU_at | 23.0503 | Mm.243014 | 0 | 92.04947 | 98.0936 | down |
| (SEQ ID NO: 1478) | | | | | | |
| WAN0088OT_at | | #N/A | | | | down |
| (SEQ ID NO: 1479) | | | | | | |
| WAN0088PY_at | 6.2069 | Mm.220038 | 3.00E−16 | 93.05556 | 12.4138 | down |
| (SEQ ID NO: 1480) | | | | | | |
| WAN0088T3_at | 71.1165 | Mm.248360 | 1E−114 | 92.13115 | 74.0291 | down |
| (SEQ ID NO: 1481) | | | | | | |
| WAN0088ZP_at | 11.1111 | Mm.336104 | 9E−53 | 91.62562 | 38.2298 | up |
| (SEQ ID NO: 1482) | | | | | | |
| WAN00895Y_f_at | | Mm.369891 | 0.001 | 91.42857 | 19.5531 | down |
| (SEQ ID NO: 1483) | | | | | | |
| WAN008C42_at | 3.05132 | Mm.18628 | 7.00E−19 | 85.71429 | 9.2233 | down |
| (SEQ ID NO: 1484) | | | | | | |
| WAN008CJ1_at | 81.9172 | Mm.2442 | 1E−170 | 94.57364 | 84.3137 | up |
| (SEQ ID NO: 1485) | | | | | | |
| WAN008DT7_at | 60.8919 | Mm.378931 | 1E−102 | 87.27273 | 66.0377 | down |
| (SEQ ID NO: 1486) | | | | | | |
| WAN008EA0_at | 99.6377 | Mm.379457 | 0 | 95.47101 | 100 | up |
| (SEQ ID NO: 1487) | | | | | | |
| WAN008F1I_at | 31.0714 | Mm.228669 | 1E−36 | 90.37037 | 24.1071 | down |
| (SEQ ID NO: 1488) | | | | | | |
| WAN008F2S_at | | #N/A | | | | down |
| (SEQ ID NO: 1489) | | | | | | |
| WAN013HX8_x_at | 98.3425 | Mm.260084 | 0 | 92.50936 | 98.3425 | up |
| (SEQ ID NO: 1490) | | | | | | |
| WAN013HXR_x_at | | Mm.336898 | 2.00E−19 | 89.62264 | 7.89278 | down |
| (SEQ ID NO: 1491) | | | | | | |
| WAN013I1P_at | 90.9474 | Mm.155896 | 0 | 96.52778 | 90.9474 | down |
| (SEQ ID NO: 1492) | | | | | | |
| WAN013I1T_x_at | | #N/A | | | | down |
| (SEQ ID NO: 1493) | | | | | | |
| WAN013I66_f_at | 57.6699 | Mm.268000 | 1E−131 | 91.84953 | 61.9417 | down |
| (SEQ ID NO: 1494) | | | | | | |
| WAN013IA0_at | 17.6727 | Mm.299381 | 4.00E−70 | 83.10811 | 18.4194 | down |
| (SEQ ID NO: 1495) | | | | | | |
| WAN013IAB_x_at | 48.8592 | #N/A | 1E−133 | 81.32045 | 48.8592 | down |
| (SEQ ID NO: 1496) | | | | | | |

TABLE 10
High Max Qp NA Known CHO Sequences

| | | | | | | | | | | | Direction of |
|---|---|---|---|---|---|---|---|---|---|---|---|
| | | | Human | | | | Mouse | | | | change (test vs |
| Qualifier List | Symbol | Title | Unigene ID | eValue | % ID | % QC | Unigene ID | eValue | % ID | % QC | control) |
| AF325501_at | LY96 | Lymphocyte | Hs.69328 | 2E−17 | 79.2727 | 74.3243 | Mm.116844 | 1E−78 | 85.20548 | 98.6486 | down |
| (SEQ ID | | antigen 96 | | | | | | | | | |
| NO: 1497) | | | | | | | | | | | |
| D45419_at | Hcfc1 | Host cell factor | Hs.83634 | 1E−22 | 84.8649 | 32.7434 | Mm.248353 | 1E−123 | 85.99291 | 99.823 | down |
| (SEQ ID | | C1 | | | | | | | | | |
| NO: 1498) | | | | | | | | | | | |
| K00924_at | VIM | Vimentin | Hs.533317 | 5E−44 | 92.9134 | 56.4444 | Mm.268000 | 3E−47 | 94.35484 | 55.1111 | down |
| (SEQ ID | | | | | | | | | | | |
| NO: 1499) | | | | | | | | | | | |
| L00176_at | HMGCR | 3-hydroxy-3- | Hs.11899 | 6E−54 | 87.9808 | 57.9387 | Mm.316652 | 4E−82 | 94.47236 | 55.4318 | up |
| (SEQ ID | | methylglutaryl- | | | | | | | | | |
| NO: 1500) | | Coenzyme A | | | | | | | | | |
| | | reductase | | | | | | | | | |
| L18986_at | LAMP1 | Lysosomal- | Hs.494419 | 1E−82 | 96.7136 | 16.2595 | Mm.16716 | 1E−160 | 87.11864 | 45.0382 | down |
| (SEQ ID | | associated | | | | | | | | | |
| NO: 1501) | | membrane | | | | | | | | | |
| | | protein 1 | | | | | | | | | |
| U48852_at | CRELD2 | Cysteine-rich | HS.211282 | 1E−109 | 81.7694 | 55.1367 | Mm.292567 | 0 | 88.79936 | 91.7221 | up |
| (SEQ ID | | with EGF-like | | | | | | | | | |
| NO: 1502) | | domains 2 | | | | | | | | | |

TABLE 11
CHO Sequences Differentially Expressed in PACE Overexpressing Cells and Encoding
ER-Associated Proteins

| | | | | | | | Mouse | | | | | Direction of |
|---|---|---|---|---|---|---|---|---|---|---|---|---|
| Qualifier List | Symbol | Title | Human Unigene ID | eValue | % ID | % QC | Unigene ID | eValue | % ID | % QC | Fold change | change |
| WAN008DRM_at | EPHX1 | Epoxide hydrolase 1, microsomal | Hs.89649 | 9E−85 | 87.987 | 60.392 | Mm.9075 | 1E−113 | 91.223 | 62.549 | 1.319 | up |
| (SEQ ID NO: 1503) | | (Ephx1) | | | | | | | | | | |
| WAN0088T7_at | Cyp51 | Cytochrome P450, family 51, | Hs.417077 | 1E−132 | 86.879 | 98.051 | Mm.46044 | 1E−152 | 88.515 | 98.441 | 1.297 | up |
| (SEQ ID NO: 1504) | | subfamily A, polypeptide 1 (CYP51A1) | | | | | | | | | | |
| WAN008ELH_at | RPN1 | Ribophorin I | Hs.518244 | 0 | 90.519 | 99.643 | Mm.188544 | 0 | 92.335 | 100.000 | 1.295 | up |
| (SEQ ID NO: 1505) | | | | | | | | | | | | |
| WAN0088K7_x_at | HSPA5 | Heat shock 70 kDa protein 5 (glucose- | | 0 | 0.000 | 0.000 | Mm.330160 | 0.000009 | 100.000 | 6.923 | 3.401 | up |
| (SEQ ID NO: 1506) | | regulated protein, 78 kDa) | | | | | | | | | | |
| L00176_at | HMGCR | 3-hydroxy-3-methylglutaryl-Coenzyme | Hs.11899 | 7E−54 | 87.981 | 57.939 | Mm.316652 | 3E−82 | 94.472 | 55.432 | 2.551 | up |
| (SEQ ID NO: 1500) | | A reductase | | | | | | | | | | |
| L00178_at | HMGCR | 3-hydroxy-3-methylglutaryl-Coenzyme | Hs.11899 | 3E−47 | 90.062 | 42.819 | Mm.316652 | 1E−57 | 93.038 | 42.021 | 2.033 | up |
| (SEQ ID NO: 1507) | | A reductase | | | | | | | | | | |
| L00169_at | HMGCR | 3-hydroxy-3-methylglutaryl-Coenzyme | Hs.11899 | 2E−19 | 89.535 | 33.992 | Mm.316652 | 7E−27 | 94.872 | 30.830 | 2.020 | up |
| (SEQ ID NO: 1508) | | A reductase | | | | | | | | | | |
| L00180_at | HMGCR | 3-hydroxy-3-methylglutary-Coenzyme | Hs.11899 | 5E−35 | 86.310 | 68.016 | Mm.316652 | 1E−49 | 90.244 | 66.397 | 1.988 | up |
| (SEQ ID NO: 1509) | | A reductase | | | | | | | | | | |
| L00181_at | HMGCR | 3-hydroxy-3-methylglutaryl-Coenzyme | Hs.11899 | 1E−37 | 89.781 | 32.697 | Mm.316652 | 3E−52 | 93.617 | 33.652 | 1.976 | up |
| (SEQ ID NO: 1510) | | A reductase | | | | | | | | | | |
| L00171_at | HMGCR | 3-hydroxy-3-methylglutaryl-Coenzyme | Hs.11899 | 5E−36 | 91.525 | 33.908 | Mm.316652 | 5E−41 | 93.220 | 33.908 | 1.934 | up |
| (SEQ ID NO: 1511) | | A reductase | | | | | | | | | | |
| L00170_x_at | HMGCR | 3-hydroxy-3-methylglutaryl-Coenzyme | Hs.11899 | 7E−27 | 89.091 | 70.968 | Mm.316652 | 6E−54 | 94.815 | 87.097 | 1.718 | up |
| (SEQ ID NO: 1512) | | A reductase | | | | | | | | | | |
| L00173_at | HMGCR | 3-hydroxy-3-methylglutaryl-Coenzyme | Hs.11899 | 2E−33 | 85.882 | 30.466 | Mm.316652 | 8E−78 | 89.919 | 44.444 | 1.621 | up |
| (SEQ ID NO: 1513) | | A reductase | | | | | | | | | | |
| L00182_at | HMGCR | 3-hydroxy-3-methylglutaryl-Coenzyme | Hs.11899 | 3E−65 | 94.012 | 48.688 | Mm.316652 | 2E−65 | 94.479 | 47.522 | 1.546 | up |
| (SEQ ID NO: 1514) | | A reductase | | | | | | | | | | |
| AF380341_at | CANX | Calnexin | Hs.567968 | 1E−101 | 93.359 | 67.016 | Mm.248827 | 1E−114 | 95.000 | 68.063 | 3.165 | up |
| (SEQ ID NO: 1515) | | | | | | | | | | | | |
| WAN013I86_x_at | CANX | Calnexin | Hs.567968 | 1E−111 | 86.053 | 97.187 | Mm.248827 | 1E−169 | 92.072 | 100.000 | 1.508 | up |
| (SEQ ID NO: 1516) | | | | | | | | | | | | |
| WAN008ES3_at | CANX | Calnexin | Hs.567968 | 5E−18 | 88.421 | 22.565 | Mm.248827 | 1E−114 | 92.562 | 86.223 | 1.376 | up |
| (SEQ ID NO: 1517) | | | | | | | | | | | | |
| WAN008EHW_at | OPRS1 | Opioid receptor, sigma 1 | Hs.522087 | 1E−141 | 87.776 | 95.777 | Mm.29025 | 1E−163 | 89.349 | 97.313 | 1.618 | up |
| (SEQ ID NO: 1518) | | | | | | | | | | | | |
| X15652_at | NSF | N-ethylmaleimide-sensitive factor | Hs.431279 | 0 | 89.899 | 99.331 | Mm.260117 | 0 | 94.649 | 100.000 | 1.346 | up |
| (SEQ ID NO: 1519) | | | | | | | | | | | | |
| WAN0088XH_at | HERPUD1 | Homocysteine-inducible, endoplasmic | Hs.146393 | 7E−79 | 87.417 | 68.481 | Mm.29151 | 1E−150 | 91.463 | 92.971 | 1.247 | up |
| (SEQ ID NO: 1520) | | reticulum stress-inducible, ubiquitin- | | | | | | | | | | |
| | | like domain member 1 | | | | | | | | | | |
| WAN008EED_at | Sc5d | Sterol-C5-desaturase (fungal ERG3, | Hs.287749 | 2E−42 | 85.446 | 40.727 | Mm.32700 | 1E−98 | 87.705 | 69.981 | 2.387 | up |
| (SEQ ID NO: 1521) | | delta-5-desaturase) homolog (S. cerevisae) | | | | | | | | | | |
| WAN008CT8_at | AP2M1 | Adaptor-related protein complex 2, mu | Hs.518460 | 0 | 94.384 | 80.803 | Mm.18946 | 0 | 95.484 | 81.152 | 1.385 | up |
| (SEQ ID NO: 1522) | | 1 subunit | | | | | | | | | | |
| WAN008CJ1_at | ERP70 | Protein disulfide isomerase- | Hs.93659 | 1E−120 | 89.628 | 81.917 | Mm.2442 | 1E−170 | 94.574 | 84.314 | 2.985 | up |
| (SEQ ID NO: 1485) | | associated 4 | | | | | | | | | | |
| WAN013I5F_at | SIAT8D | ST8 alpha-N-acetyl-neuraminide | Hs.308628 | 0 | 90.621 | 61.296 | Mm.306228 | 0 | 90.673 | 90.283 | 1.370 | up |
| (SEQ ID NO: 1523) | | alpha-2,8-sialyltransferase 4 | | | | | | | | | | |
| WAN0088XZ_at | RTN3 | Reticulon 3 | Hs.473761 | 1E−167 | 91.126 | 83.848 | Mm.246990 | 0 | 92.545 | 99.819 | 1.546 | up |
| (SEQ ID NO: 1524) | | | | | | | | | | | | |
| WAN013I9D_at | HYOU1 | Hypoxia up-regulated 1 | Hs.277704 | 4E−72 | 85.498 | 26.417 | Mm.116721 | 1E−122 | 92.236 | 25.698 | 1.695 | up |
| (SEQ ID NO: 1525) | | | | | | | | | | | | |
| WAN008DUB_at | RDH11 | Retinol dehydrogenase 11 (Rdh11) | Hs.226007 | 1E−77 | 84.840 | 81.385 | Mm.291799 | 3E−89 | 91.373 | 55.195 | 1.414 | up |
| (SEQ ID NO: 1526) | | | | | | | | | | | | |
| WAN008ELW_f_at | Sec13I1 | SEC13-like 1 (S. cerevisiae) | Hs.166924 | 8E−22 | 93.151 | 87.952 | Mm.29296 | 8E−24 | 92.500 | 96.386 | 1.302 | up |
| (SEQ ID NO: 1527) | | | | | | | | | | | | |
| WAN013I30_at | TRA1 | Tumor rejection antigen (gp96) 1 | Hs.192374 | 0 | 90.545 | 100.000 | Mm.87773 | 0 | 93.273 | 100.000 | 2.849 | up |
| (SEQ ID NO: 1528) | | | | | | | | | | | | |
| WAN013HWO_x_at | TRA1 | Tumor rejection antigen (gp96) 1 | Hs.192374 | 6E−51 | 85.106 | 94.000 | Mm.87773 | 2E−66 | 87.805 | 98.400 | 2.092 | up |
| (SEQ ID NO: 1529) | | | | | | | | | | | | |
| WAN0088ZO_x_at | SYNCRIP | Synaptotagmin binding, cytoplasmic | Hs.472056 | 1E−50 | 94.615 | 70.652 | Mm.32874 | 3E−56 | 95.556 | 73.370 | 2.198 | up |
| (SEQ ID NO: 1530) | | RNA interacting protein | | | | | | | | | | |
| WAN00894J_at | ZMPSTE24 | Zinc metallopeptidase (STE24 | Hs.591501 | 1E−146 | 88.727 | 93.922 | Mm.34399 | 0 | 92.277 | 99.020 | 1.484 | up |
| (SEQ ID NO: 1531) | | homolog, yeast) | | | | | | | | | | |
| WAN013I4D_at | TAP2 | Transporter 2, ATP-binding cassette, | Hs.502 | 5E−33 | 83.258 | 46.331 | Mm.14814 | 1E−111 | 88.950 | 75.891 | −1.914 | down |
| (SEQ ID NO: 1532) | | sub-family B (MDR/TAP) | | | | | | | | | | |
| AF323965_at | CYP11A1 | Cytochrome P450, family 11, | Hs.303980 | 1E−175 | 82.511 | 86.034 | Mm.302865 | 0 | 91.004 | 88.349 | −1.492 | down |
| (SEQ ID NO: 1533) | | subfamily A, polypeptide 1 | | | | | | | | | | |
| WAN013HX5_at | MGST1 | Microsomal glutathione S-transferase 1 | Hs.389700 | 3E−28 | 78.987 | 81.950 | Mm.14796 | 1E−152 | 89.394 | 95.851 | −1.483 | down |
| (SEQ ID NO: 1534) | | | | | | | | | | | | |
| AJ298842_at | Dyt1 | Torsin family 1, member A (torsin A) | Hs.534312 | 2E−89 | 87.209 | 58.703 | Mm.154994 | 1E−153 | 94.366 | 60.580 | −1.251 | down |
| (SEQ ID NO: 1535) | | | | | | | | | | | | |
| AF004831_at | SPTLC1 | Serine palmitoyltransferase, long | Hs.90458 | 1E−18 | 88.889 | 6.767 | Mm.240336 | 5E−84 | 89.441 | 24.211 | −2.201 | down |
| (SEQ ID NO: 1536) | | chain base subunit 1 | | | | | | | | | | |
| WAN013I4M_at | ENTPD5 | Ectonucleoside triphosphate | Hs.131555 | 2E−48 | 90.341 | 32.653 | Mm.10211 | 8E−44 | 88.298 | 34.879 | −1.618 | down |
| (SEQ ID NO: 1537) | | diphosphohydrolase 5 | | | | | | | | | | |
| WAN013I65_at | DPAGT1 | Dolichyl-phosphate (UDP-N- | Hs.524081 | 0 | 90.962 | 39.334 | Mm.18353 | 1E−178 | 90.267 | 39.637 | −1.455 | down |
| (SEQ ID NO: 1538) | | acetylglucosamine) N- | | | | | | | | | | |
| | | acetylglucosaminephosphotransferase | | | | | | | | | | |
| | | 1 (GlcNAc-1-P transferase) | | | | | | | | | | |
| WAN0088KG_at | PPGB | Protective protein for beta- | Hs.517076 | 1E−115 | 87.589 | 72.870 | Mm.359633 | 1E−149 | 90.931 | 72.870 | −3.26 | down |
| (SEQ ID NO: 1539) | | galactosidase (galactosialidosis) | | | | | | | | | | |
| WAN0088TG_at | SRP72 | Signal recognition particle 72 kDa | Hs.237825 | 1E−58 | 89.041 | 50.812 | Mm.296976 | 1E−119 | 92.401 | 76.334 | −1.416 | down |
| (SEQ ID NO: 1540) | | | | | | | | | | | | |
| WAN013I39_at | GGA2 | Golgi associated, gamma adaptin ear | Hs.460336 | 7E−30 | 84.454 | 46.667 | Mm.29619 | 1E−147 | 93.333 | 79.412 | −1.474 | down |
| (SEQ ID NO: 1541) | | containing, ARF binding protein 2 | | | | | | | | | | |
| WAN008CUO_at | GGA2 | Golgi associated, gamma adaptin ear | Hs.460336 | 4E−31 | 89.344 | 25.957 | Mm.29619 | 1E−146 | 90.364 | 99.362 | −1.253 | down |
| (SEQ ID NO: 1542) | | containing, ARF binding protein 2 | | | | | | | | | | |

TABLE 12
CHO Sequences Differentially Expressed in PACE Overexpressing Cells and Encoding
Golgi-Associated Proteins

| | | | Human | | |
|---|---|---|---|---|---|
| | | | Unigene | | |
| Qualifier List | Symbol | Title | ID | eValue | % ID |
| WAN0088ZC_at | PSEN1 | Presenilin 1 (Alzheimer disease 3) | Hs.592324 | 5E−82 | 89.161 |
| (SEQ ID NO: 1543) | | | | | |
| WAN014IYT_at | FURIN | Furin (paired basic amino acid | Hs.513153 | 0 | 99.922 |
| (SEQ ID NO: 1544) | | cleaving enzyme) | | | |
| WAN008CMC_x_at | MAPRE1 | Microtubule-associated protein, | Hs.472437 | 2E−49 | 94.964 |
| (SEQ ID NO: 1545) | | RP/EB family, member 1 | | | |
| WAN008D2C_at | Csnk2a2 | Casein kinase II, alpha 2, polypeptide | Hs.82201 | 1E−141 | 89.293 |
| (SEQ ID NO: 1546) | | (Csnk2a2) | | | |
| WAN008E72_x_at | GDI2 | GDP dissociation inhibitor 2 | Hs.299055 | 6E−25 | 86.139 |
| (SEQ ID NO: 1547) | | | | | |
| WAN013I8H_x_at | APP | Amyloid beta (A4) precursor protein | Hs.434980 | 1E−102 | 84.192 |
| (SEQ ID NO: 1548) | | (protease nexin-II, Alzheimer disease) | | | |
| AF030413_at | APP | Amyloid beta (A4) precursor protein | Hs.434980 | 6E−90 | 93.421 |
| (SEQ ID NO: 1549) | | (protease nexin-II, Alzheimer disease) | | | |
| WAN013I12_at | VDP | Vesicle docking protein p115 | Hs.292689 | 4E−12 | 85.227 |
| (SEQ ID NO: 1550) | | | | | |
| WAN013HUW_at | ARL1 | ADP-ribosylation factor-like 1 | Hs.372616 | 2E−88 | 91.339 |
| (SEQ ID NO: 1551) | | | | | |
| WAN008CLK_at | Rab6 | RAB6, member RAS oncogene family | Hs.503222 | 2E−55 | 88.477 |
| (SEQ ID NO: 1552) | | | | | |
| WAN0088X9_at | RAB34 | RAB34, member RAS oncogene | Hs.301853 | 1E−108 | 89.174 |
| (SEQ ID NO: 1553) | | family | | | |
| WAN013HZH_at | M6PRBP1 | Mannose-6-phosphate receptor | Hs.140452 | 3E−18 | 76.364 |
| (SEQ ID NO: 1554) | | binding protein 1 | | | |

| | | | | | | | Direction |
|---|---|---|---|---|---|---|---|
| | | Mouse | | | | Fold | of |
| Qualifier List | % QC | Unigene ID | eValue | % ID | % QC | change | change |
| WAN0088ZC_at | 86.145 | Mm.998 | 5E−78 | 88.153 | 86.446 | 1.255 | up |
| (SEQ ID NO: 1543) | | | | | | | |
| WAN014IYT_at | 93.193 | Mm.5241 | 0 | 88.701 | 89.718 | 2.500 | up |
| (SEQ ID NO: 1544) | | | | | | | |
| WAN008CMC_x_at | 88.535 | Mm.143877 | 9E−50 | 94.964 | 88.535 | 1.242 | up |
| (SEQ ID NO: 1545) | | | | | | | |
| WAN008D2C_at | 91.497 | Mm.51136 | 0 | 95.158 | 99.261 | 1.272 | up |
| (SEQ ID NO: 1546) | | | | | | | |
| WAN008E72_x_at | 100.000 | Mm.153226 | 1E−74 | 95.545 | 100.000 | −1.455 | down |
| (SEQ ID NO: 1547) | | | | | | | |
| WAN013I8H_x_at | 99.646 | Mm.277585 | 1E−162 | 87.788 | 100.000 | −1.902 | down |
| (SEQ ID NO: 1548) | | | | | | | |
| AF030413_at | 100.000 | Mm.277585 | 2E−97 | 94.737 | 100.000 | −1.851 | down |
| (SEQ ID NO: 1549) | | | | | | | |
| WAN013I12_at | 15.385 | Mm.15868 | 9E−42 | 85.259 | 43.881 | −1.588 | down |
| (SEQ ID NO: 1550) | | | | | | | |
| WAN013HUW_at | 51.313 | Mm.291247 | 1E−150 | 90.798 | 98.788 | −1.687 | down |
| (SEQ ID NO: 1551) | | | | | | | |
| WAN008CLK_at | 48.214 | Mm.28650 | 1E−163 | 92.276 | 97.619 | −1.292 | down |
| (SEQ ID NO: 1552) | | | | | | | |
| WAN0088X9_at | 66.730 | Mm.275864 | 1E−161 | 92.157 | 87.262 | −1.374 | down |
| (SEQ ID NO: 1553) | | | | | | | |
| WAN013HZH_at | 47.826 | Mm.311696 | 1E−107 | 81.239 | 98.261 | −1.342 | down |
| (SEQ ID NO: 1554) | | | | | | | |

### Example 7

### Genes Differentially Expressed in Cells with High Cellular Growth Rate

A summary of nucleic acids identified as differentially expressed in cells with high cellular growth rate is provided in Tables 13 and 14. For each nucleic acid, a qualifier name, symbol, and title are provided, as well as whether the nucleic acid is up-regulated or down-regulated in the cells with higher maximum cellular productivity. For nucleic acids with human or mouse homologs in the Unigene database, the table provides Unigene ID numbers and statistics relating to the comparison, including e-values, percent sequence identities between the CHO sequence and the Unigene databank entries, and percent coverage (“% QC”).

TABLE 13
High Cell Growth Rate NA Unknown CHO Sequences

| | | | Human | |
|---|---|---|---|---|
| Qualifier List | Symbol | Title | Unigene ID | eValue |
| WAN0088JV_at | TRIB3 | Tribbles homolog 3 (Drosophila) | Hs.516826 | 4E−62 |
| (SEQ ID NO: 1555) | | | | |
| WAN0088PT_at | Psmc1 | Protease (prosome, macropain) 26S | Hs.356654 | 0 |
| (SEQ ID NO: 1556) | | subunit, ATPase 1 | | |
| WAN0088XH_at | HERPUD1 | Homocysteine-inducible, endoplasmic | Hs.146393 | 7E−79 |
| (SEQ ID NO: 1557) | | reticulum stress-inducible, ubiquitin-like | | |
| | | domain member 1 | | |
| WAN008BSH_at | CAT | Catalase | Hs.502302 | 6E−16 |
| (SEQ ID NO: 1558) | | | | |
| WAN008CM1_x_at | DQ390542.2 | Mitochondrial 12S ribosomal RNA | #N/A | |
| (SEQ ID NO: 1559) | | | | |
| WAN008CWC_x_at | NA | WAN008CWC 10603C-F10 | #N/A | |
| (SEQ ID NO: 1560) | | | | |
| WAN008D2Q_at | Eif4b | Eukaryotic translation initiation factor 4B | #N/A | 6E−49 |
| (SEQ ID NO: 1561) | | (Eif4b) | | |
| WAN008D5V_x_at | Gosr2 | Golgi SNAP receptor complex member 2, | Hs.463278 | |
| (SEQ ID NO: 1562) | | mRNA (cDNA clone MGC: 6437 | | |
| | | IMAGE: 3601627) | | |
| WAN008D6J_at | HMGA2 | High mobility group AT-hook 2 | Hs.505924 | 4E−62 |
| (SEQ ID NO: 1563) | | | | |
| WAN008DGD_at | Aplp2 | Amyloid beta (A4) precursor-like protein 2 | #N/A | |
| (SEQ ID NO: 1564) | | (Aplp2) | | |
| WAN008DJ9_at | SLC1A4 | Solute carrier family 1 (glutamate/neutral | Hs.323878 | 2E−39 |
| (SEQ ID NO: 1565) | | amino acid transporter), member 4 | | |
| WAN008DSE_at | SLC1A4 | Solute carrier family 1 (glutamate/neutral | Hs.323878 | 2E−82 |
| (SEQ ID NO: 1566) | | amino acid transporter), member 4 | | |
| WAN008E2E_at | PSMC4 | Proteasome (prosome, macropain) 26S | Hs.211594 | 1E−131 |
| (SEQ ID NO: 1567) | | subunit, ATPase, 4 | | |
| WAN008E8M_at | HADHB | Hydroxyacyl-Coenzyme A | Hs.515848 | 1E−114 |
| (SEQ ID NO: 1568) | | dehydrogenase/3-ketoacyl-Coenzyme A | | |
| | | thiolase/enoyl-Coenzyme A hydratase | | |
| | | (trifunctional protein), beta subunit | | |
| WAN008EBJ_at | Triobp | TRIO and F-actin binding protein | Hs.533030 | 6E−89 |
| (SEQ ID NO: 1569) | | | | |
| WAN008EFS_at | TXNRD1 | Thioredoxin reductase 1 | Hs.434367 | 6E−18 |
| (SEQ ID NO: 1570) | | | | |
| WAN008EGV_at | GDI2 | GDP dissociation inhibitor 2 | Hs.299055 | 0 |
| (SEQ ID NO: 1571) | | | | |
| WAN008EMQ_at | KPNA3 | Karyopherin alpha 3 (importin alpha 4) | Hs.527919 | 1E−144 |
| (SEQ ID NO: 1572) | | | | |
| WAN008ERL_at | ETFA | TYRO3P protein tyrosine kinase | Hs.39925 | 1E−135 |
| (SEQ ID NO: 1573) | | pseudogene | | |
| WAN008ETP_at | AADACL1 | Arylacetamide deacetylase-like 1 | Hs.444099 | 2E−72 |
| (SEQ ID NO: 1574) | | | | |
| WAN008EX2_x_at | IFRD1 | Interferon-related developmental regulator 1 | Hs.7879 | 7E−39 |
| (SEQ ID NO: 1575) | | | | |
| WAN013HUM_at | EHD4 | EH-domain containing 4 | Hs.143703 | 1E−95 |
| (SEQ ID NO: 1576) | | | | |
| WAN013HWG_at | DQ390542.2 | Mitochondrial NADH dehydrogenase | Hs.550202 | 5.00E−08 |
| (SEQ ID NO: 1577) | | subunit 5 | | |
| WAN013HX4_at | ESD | Esterase D/formylglutathione hydrolase | Hs.432491 | 1E−155 |
| (SEQ ID NO: 1578) | | | | |
| WAN013HYO_at | RPL11 | Ribosomal protein L11 | Hs.388664 | 0 |
| (SEQ ID NO: 1579) | | | | |
| WAN013I0W_at | TAPBP | TAP binding protein (tapasin) | Hs.370937 | 2E−57 |
| (SEQ ID NO: 1580) | | | | |
| WAN013I0X_at | GSS | Glutathione synthetase | Hs.82327 | 3E−96 |
| (SEQ ID NO: 1581) | | | | |
| WAN013I1G_at | SLC25A20 | Solute carrier family 25 | Hs.13845 | 1E−137 |
| (SEQ ID NO: 1582) | | (carnitine/acylcarnitine translocase), | | |
| | | member 20 | | |
| WAN013I38_at | Pkm2 | Pyruvate kinase, muscle, mRNA (cDNA | Hs.198281 | 3E−63 |
| (SEQ ID NO: 1583) | | clone MGC: 11908 IMAGE: 3598842) | | |
| WAN013I8K_at | DQ390542.2 | Mitochondrial cytochrome b | #N/A | |
| (SEQ ID NO: 1584) | | | | |

| | | | Mouse | | | | |
|---|---|---|---|---|---|---|---|
| Qualifier List | % ID | % QC | Unigene ID | eValue | % ID | % QC | |
| WAN0088JV_at | 81.778 | 86.37 | Mm.276018 | 1E−158 | 88.845 | 98.08 | down |
| (SEQ ID NO: 1555) | | | | | | | |
| WAN0088PT_at | 92.292 | 99.59 | Mm.157105 | 0 | 94.561 | 99.17 | down |
| (SEQ ID NO: 1556) | | | | | | | |
| WAN0088XH_at | 87.417 | 68.48 | Mm.29151 | 1E−144 | 91.463 | 92.97 | down |
| (SEQ ID NO: 1557) | | | | | | | |
| WAN008BSH_at | 89.286 | 38.71 | Mm.4215 | 3E−41 | 89.247 | 85.71 | down |
| (SEQ ID NO: 1558) | | | | | | | |
| WAN008CM1_x_at | | | #N/A | 0 | 0 | 0 | up |
| (SEQ ID NO: 1559) | | | | | | | |
| WAN008CWC_x_at | | | #N/A | | | | up |
| (SEQ ID NO: 1560) | | | | | | | |
| WAN008D2Q_at | 92.949 | 29.38 | Mm.290022 | 1E−129 | 91.316 | 71.56 | up |
| (SEQ ID NO: 1561) | | | | | | | |
| WAN008D5V_x_at | | | Mm.195451 | 1E−08 | 90.196 | 43.59 | down |
| (SEQ ID NO: 1562) | | | | | | | |
| WAN008D6J_at | 94.805 | 33.33 | Mm.157190 | 1E−130 | 90.444 | 97.4 | down |
| (SEQ ID NO: 1563) | | | | | | | |
| WAN008DGD_at | | | Mm.19133 | 7E−69 | 93.088 | 44.47 | down |
| (SEQ ID NO: 1564) | | | | | | | |
| WAN008DJ9_at | 86.932 | 39.46 | Mm.6379 | 1E−121 | 88.614 | 90.58 | down |
| (SEQ ID NO: 1565) | | | | | | | |
| WAN008DSE_at | 86.89 | 60.18 | Mm.6379 | 1E−117 | 90.643 | 62.75 | down |
| (SEQ ID NO: 1566) | | | | | | | |
| WAN008E2E_at | 89.95 | 100 | Mm.29582 | 1E−141 | 90.955 | 100 | down |
| (SEQ ID NO: 1567) | | | | | | | |
| WAN008E8M_at | 88.191 | 85.78 | Mm.291463 | 1E−162 | 91.256 | 96.12 | down |
| (SEQ ID NO: 1568) | | | | | | | |
| WAN008EBJ_at | 87.613 | 61.87 | Mm.123714 | 1E−175 | 90.707 | 92.52 | down |
| (SEQ ID NO: 1569) | | | | | | | |
| WAN008EFS_at | 86.777 | 22.08 | Mm.210155 | 2E−54 | 88.393 | 40.88 | down |
| (SEQ ID NO: 1570) | | | | | | | |
| WAN008EGV_at | 92.897 | 94.86 | Mm.8070 | 0 | 94.019 | 94.86 | down |
| (SEQ ID NO: 1571) | | | | | | | |
| WAN008EMQ_at | 91.853 | 100 | Mm.25548 | 0 | 96.334 | 100 | down |
| (SEQ ID NO: 1572) | | | | | | | |
| WAN008ERL_at | 95.57 | 87.05 | | 1E−162 | 95.868 | 100 | down |
| (SEQ ID NO: 1573) | | | | | | | |
| WAN008ETP_at | 86.942 | 97.65 | Mm.24576 | 8E−89 | 88.926 | 100 | down |
| (SEQ ID NO: 1574) | | | | | | | |
| WAN008EX2_x_at | 90.299 | 100 | Mm.168 | 6E−63 | 97.761 | 100 | down |
| (SEQ ID NO: 1575) | | | | | | | |
| WAN013HUM_at | 92.771 | 59 | Mm.132226 | 1E−132 | 89.286 | 99.53 | down |
| (SEQ ID NO: 1576) | | | | | | | |
| WAN013HWG_at | 85 | 6.525 | #N/A | | | | up |
| (SEQ ID NO: 1577) | | | | | | | |
| WAN013HX4_at | 87.617 | 93.51 | Mm.38055 | 0 | 91.902 | 93.16 | down |
| (SEQ ID NO: 1578) | | | | | | | |
| WAN013HYO_at | 90.522 | 100 | Mm.276856 | 0 | 91.473 | 99.81 | up |
| (SEQ ID NO: 1579) | | | | | | | |
| WAN013I0W_at | 80.633 | 93.23 | Mm.154457 | 1E−149 | 86.885 | 95.31 | down |
| (SEQ ID NO: 1580) | | | | | | | |
| WAN013I0X_at | 90.444 | 56.13 | Mm.252316 | 1E−129 | 95.189 | 55.75 | down |
| (SEQ ID NO: 1581) | | | | | | | |
| WAN013I1G_at | 86.706 | 87.65 | Mm.29666 | 0 | 92.354 | 86.43 | down |
| (SEQ ID NO: 1582) | | | | | | | |
| WAN013I38_at | 90.521 | 38.36 | Mm.216135 | 0 | 92.99 | 88.18 | down |
| (SEQ ID NO: 1583) | | | | | | | |
| WAN013I8K_at | | | #N/A | | | | up |
| (SEQ ID NO: 1584) | | | | | | | |

TABLE 14
High Cell Growth Rate NA Known CHO Sequences

| | | | Human | | | | Mouse | | | | |
|---|---|---|---|---|---|---|---|---|---|---|---|
| Qualifier List | Symbol | Title | Unigene ID | eValue | % ID | % QC | Unigene ID | eValue | % ID | % QC | |
| AF081143_at | RPS18 | Ribosomal protein S18 | Hs.546290 | 1E−78 | 90.717 | 98.34 | Mm.324762 | 4E−93 | 92.946 | 100 | up |
| (SEQ ID NO: 1585) | | | | | | | | | | | |
| U62588_x_at | SDC1 | Syndecan 1 | Hs.224607 | 1E−32 | 93 | 53.19 | Mm.2580 | 7E−48 | 90.85 | 81.38 | down |
| (SEQ ID NO: 1586) | | | | | | | | | | | |
| X51747_at | HSPB1 | Heat shock 27 kDa | Hs.520973 | 1E−101 | 87.368 | 50.53 | Mm.13849 | 0 | 91.952 | 66.09 | up |
| (SEQ ID NO: 1587) | | protein 1 | | | | | | | | | |

### Example 8

### Genes Differentially Expressed in Cells with High Peak Cell Density

A summary of nucleic acids identified as differentially expressed in cells with high peak cell density is provided in Tables 15, 16, and 17. For each nucleic acid, a qualifier name, symbol, and title are provided, as well as whether the nucleic acid is up-regulated or down-regulated in the cells with higher maximum cellular productivity. For nucleic acids with human or mouse homologs in the Unigene database, the table provides Unigene ID numbers and statistics relating to the comparison, including e-values, percent sequence identities between the CHO sequence and the Unigene databank entries, and percent coverage (“% QC”).

TABLE 15
High Cell Density Unknown CHO Sequences

| | | | | | | | | | | | Direction of |
|---|---|---|---|---|---|---|---|---|---|---|---|
| | | | Human | | | | Mouse | | | | change |
| Qualifier List | Symbol | Title | Unigene ID | eValue | % ID | % QC | Unigene ID | eValue | % ID | % QC | (test vs control) |
| WAN0088J9_x_at | CCNA2 | cyclin A2 | Hs.85137 | 1.00E−55 | 87.6494 | 20.291 | Mm.4189 | 4.00E−60 | 89.423 | 16.815 | up |
| (SEQ ID | | | | | | | | | | | |
| NO: 1588) | | | | | | | | | | | |
| WAN0088PR_at | CCPG1 | Phosphatidylinositol glycan, class B | Hs.285051 | 2E−08 | 87.5 | 11.429 | Mm.268475 | 3E−34 | 90.244 | 21.964 | down |
| (SEQ ID | | | | | | | | | | | |
| NO: 1589) | | | | | | | | | | | |
| WAN0088Q6_at | Hist1h2bn | Histone 1, H2bn | Hs.534368 | 1E−153 | 93.7158 | 63.432 | Mm.261676 | 0 | 95.134 | 71.231 | down |
| (SEQ ID | | | | | | | | | | | |
| NO: 1590) | | | | | | | | | | | |
| WAN0088S8_at | SLC29A1 | Solute carrier family 29 | Hs.25450 | 3E−35 | 81.3559 | 76.129 | Mm.29744 | 6E−97 | 86.098 | 88.172 | up |
| (SEQ ID | | (nucleoside transporters), | | | | | | | | | |
| NO: 1591) | | member 1 | | | | | | | | | |
| WAN0088T2_at | ATF4 | Activating transcription factor 4 | Hs.496487 | 1E−158 | 88.5397 | 97.83 | Mm.641 | 0 | 91.714 | 96.022 | up |
| (SEQ ID | | (tax-responsive enhancer element B67) | | | | | | | | | |
| NO: 1592) | | | | | | | | | | | |
| WAN0088X2_at | PEO1 | Progressive external | Hs.22678 | 1E−141 | 88.651 | 94.534 | Mm.105585 | 7E−78 | 90.678 | 47.773 | up |
| (SEQ ID | | ophthalmoplegia 1 | | | | | | | | | |
| NO: 1593) | | | | | | | | | | | |
| WAN008BRK_at | Tmsb4x | Thymosin, beta 4, X | Hs.522584 | 1E−153 | 93.617 | 71.756 | Mm.142729 | 0 | 95.34 | 98.282 | down |
| (SEQ ID | | chromosome | | | | | | | | | |
| NO: 1594) | | | | | | | | | | | |
| WAN008BSG_x_at | TRAM1 | Translocation associated | Hs.491988 | 7E−29 | 89.9225 | 36.236 | Mm.28765 | 5E−44 | 91.787 | 58.146 | up |
| (SEQ ID | | membrane protein 1 | | | | | | | | | |
| NO: 1595) | | | | | | | | | | | |
| WAN008CHP_x_at | NA | WAN008CHP 10599D-H02 | #N/A | | | | #N/A | | | | up |
| (SEQ ID | | | | | | | | | | | |
| NO: 1596) | | | | | | | | | | | |
| WAN008CM7_x_at | MRPL51 | Mitochondrial ribosomal protein | Hs.55847 | 0.0002 | 82.0225 | 25.356 | Mm.354426 | 6E−13 | 84.466 | 29.345 | up |
| (SEQ ID | | L51 | | | | | | | | | |
| NO: 1597) | | | | | | | | | | | |
| WAN008CQP_at | AATF | Apoptosis antagonizing | Hs.195740 | 6E−73 | 83.7587 | 99.309 | Mm.257482 | 8E−99 | 85.615 | 99.309 | up |
| (SEQ ID | | transcription factor | | | | | | | | | |
| NO: 1598) | | | | | | | | | | | |
| WAN008CX9_at | ISGF3G | Interferon-stimulated | Hs.1706 | 2E−64 | 83.4225 | 81.481 | Mm.2032 | 1E−119 | 88.424 | 88.453 | up |
| (SEQ ID | | transcription factor 3, gamma | | | | | | | | | |
| NO: 1599) | | 48 kDa | | | | | | | | | |
| WAN008CXC_at | ATP6V0A1 | ATPase, H+ transporting, | Hs.463074 | 0 | 93.2927 | 99.394 | Mm.340818 | 0 | 94.343 | 100 | down |
| (SEQ ID | | lysosomal V0 subunit a isoform 1 | | | | | | | | | |
| NO: 1600) | | | | | | | | | | | |
| WAN008D2S_at | BPY2IP1 | BPY2 interacting protein 1 | Hs.66048 | 6E−15 | 84.0336 | 20.951 | Mm.248559 | 1E−101 | 86.99 | 69.014 | down |
| (SEQ ID | | | | | | | | | | | |
| NO: 1601) | | | | | | | | | | | |
| WAN008D3Z_at | GALNT7 | UDP-N-acetyl-alpha-D- | Hs.127407 | 1E−135 | 88.8668 | 100 | Mm.62886 | 1E−150 | 90.855 | 100 | down |
| (SEQ ID | | galactosamine:polypeptide N- | | | | | | | | | |
| NO: 1602) | | acetylgalactosaminyltransferase 7 (GalNAc-T7) | | | | | | | | | |
| WAN008D55-rc_at | LAMB1 | Laminin, beta 1 | Hs.489646 | 1E−155 | 87.8229 | 97.482 | Mm.172674 | 1E−161 | 91.667 | 77.698 | 1up, 1down |
| (SEQ ID | | | | | | | | | | | |
| NO: 1603) | | | | | | | | | | | |
| WAN008D5V_x_at | Gosr2 | Golgi SNAP receptor complex | Hs.463278 | | | | Mm.195451 | 1E−08 | 90.196 | 43.59 | down |
| (SEQ ID | | member 2, mRNA (cDNA clone | | | | | | | | | |
| NO: 1562) | | MGC: 6437 IMAGE: 3601627) | | | | | | | | | |
| WAN008D6R_at | TMED4 | Transmembrane emp24 protein | Hs.510745 | 1E−111 | 91.0828 | 73.709 | Mm.254495 | 1E−140 | 92.412 | 86.62 | down |
| (SEQ ID | | transport domain containing 4 | | | | | | | | | |
| NO: 1604) | | | | | | | | | | | |
| WAN008DFT_at | ABHD6 | Abhydrolase domain containing 6 | Hs.476454 | 3E−17 | 83.2168 | 26.335 | Mm.181473 | 9E−53 | 87.124 | 42.91 | up |
| (SEQ ID | | | | | | | | | | | |
| NO: 1605) | | | | | | | | | | | |
| WAN008DGZ_at | SLC7A6OS | Solute carrier family 7, member | Hs.334848 | 2E−79 | 84.2342 | 79.428 | Mm.269029 | 1E−139 | 89.862 | 77.639 | up |
| (SEQ ID | | 6 opposite strand | | | | | | | | | |
| NO: 1606) | | | | | | | | | | | |
| WAN008DI7_at | FBXO42 | F-box protein 42 | #N/A | | | | Mm.28865 | 2E−23 | 86.957 | 68.452 | down |
| (SEQ ID | | | | | | | | | | | |
| NO: 1607) | | | | | | | | | | | |
| WAN008DIA_at | U2AF1 | U2(RNU2) small nuclear RNA | Hs.365116 | 1E−170 | 90.5544 | 97.4 | Mm.311063 | 0 | 95.4 | 100 | up |
| (SEQ ID | | auxiliary factor 1 | | | | | | | | | |
| NO: 1608) | | | | | | | | | | | |
| WAN008DJ8_f_at | Ubc | Ubiquitin C, mRNA (cDNA | Hs.378821 | 1E−22 | 87.8049 | 24.848 | Mm.331 | 2E−25 | 88.618 | 24.848 | down |
| (SEQ ID | | clone IMAGE: 2645223) | | | | | | | | | |
| NO: 1609) | | | | | | | | | | | |
| WAN008DMI_at | ACSL5 | Acyl-CoA synthetase long-chain | Hs.11638 | 1E−118 | 85 | 96.601 | #N/A | 0 | 89.946 | 99.642 | up |
| (SEQ ID | | family member 5 | | | | | | | | | |
| NO: 1610) | | | | | | | | | | | |
| WAN008DMJ_at | NAB2 | NGFI-A binding protein 2 | Hs.159223 | 1E−176 | 89.5717 | 100 | Mm.336898 | 0 | 92.683 | 99.255 | up |
| (SEQ ID | | (EGR1 binding protein 2) | | | | | | | | | |
| NO: 1611) | | | | | | | | | | | |
| WAN008DQE_at | YES1 | V-yes-1 Yamaguchi sarcoma | Hs.194148 | 0 | 94.8529 | 100 | #N/A | 0 | 95.588 | 100 | up |
| (SEQ ID | | viral oncogene homolog 1 | | | | | | | | | |
| NO: 1612) | | | | | | | | | | | |
| WAN008DS9_at | CFL2 | Cofilin 2 (muscle) | Hs.180141 | 1E−113 | 89.0187 | 90.87 | Mm.276826 | 1E−132 | 92.982 | 84.713 | down |
| (SEQ ID | | | | | | | | | | | |
| NO: 1613) | | | | | | | | | | | |
| WAN008DWJ_at | USP1 | Similar to ubiquitin specific | Hs.35086 | 0 | 92.9476 | 97.018 | Mm.371692 | 0 | 94.182 | 96.491 | up |
| (SEQ ID | | protease 1 | | | | | | | | | |
| NO: 1614) | | | | | | | | | | | |
| WAN008DZF_at | AL033326 | Expressed sequence AL033326 | #N/A | 1E−92 | 87.0558 | 99.747 | Mm.182145 | 1E−156 | 92.658 | 100 | down |
| (SEQ ID | | | | | | | | | | | |
| NO: 1615) | | | | | | | | | | | |
| WAN008E06_at | Rabep2 | Rabaptin, RAB GTPase binding | Hs.555978 | 2E−92 | 85.4722 | 76.34 | Mm.35467 | 0 | 91.37 | 98.521 | down |
| (SEQ ID | | effector protein 2 | | | | | | | | | |
| NO: 1616) | | | | | | | | | | | |
| WAN008E1M_f_at | CD36 | CD36 antigen | Hs.120949 | 0.00001 | 90.9091 | 3.0513 | Mm.18628 | 7.00E−19 | 85.714 | 9.2233 | down |
| (SEQ ID | | | | | | | | | | | |
| NO: 1617) | | | | | | | | | | | |
| WAN008E2Q_at | GSPT1 | G1 to S phase transition 1 | Hs.528780 | 0 | 93.6957 | 100 | Mm.325827 | 0 | 95.87 | 100 | up |
| (SEQ ID | | | | | | | | | | | |
| NO: 1618) | | | | | | | | | | | |
| WAN008E5L_at | SLC1A5 | Solute carrier family 1 (neutral | Hs.515494 | 8E−42 | 84.1667 | 45.627 | Mm.1056 | 1E−115 | 87.671 | 83.27 | up |
| (SEQ ID | | amino acid transporter), member 5 | | | | | | | | | |
| NO: 1619) | | | | | | | | | | | |
| WAN008E9N_at | KLHL7 | Kelch-like 7 (Drosophila) | Hs.385861 | 1E−150 | 89.4422 | 99.406 | Mm.273768 | 0 | 93.111 | 89.109 | down |
| (SEQ ID | | | | | | | | | | | |
| NO: 1620) | | | | | | | | | | | |
| WAN008EBP_at | Sqstm1 | Sequestosome 1 | Hs.529892 | 0 | 93.4066 | 97.849 | Mm.40828 | 0 | 93.407 | 97.849 | down |
| (SEQ ID | | | | | | | | | | | |
| NO: 1621) | | | | | | | | | | | |
| WAN008EH5_at | PRNP | Prion protein (p27-30) | Hs.472010 | 9E−45 | 86.802 | 34.806 | Mm.648 | 4E−92 | 89.815 | 57.24 | down |
| (SEQ ID | | (Creutzfeld-Jakob disease, | | | | | | | | | |
| NO: 1622) | | Gerstmann-Strausler-Scheinker | | | | | | | | | |
| | | syndrome, fatal familial | | | | | | | | | |
| | | insomnia) | | | | | | | | | |
| WAN008EID_at | TRIB3 | Tribbles homolog 3 (Drosophila) | Hs.516826 | 1E−12 | 92.7273 | 12.195 | Mm.276018 | 1E−51 | 89.773 | 39.024 | up |
| (SEQ ID | | | | | | | | | | | |
| NO: 1623) | | | | | | | | | | | |
| WAN008EJY_at | NA | WAN008EJY 11232A-H04 | #N/A | | | | #N/A | | | | up |
| (SEQ ID | | | | | | | | | | | |
| NO: 1624) | | | | | | | | | | | |
| WAN008EKK_at | PSMA8 | Proteasome (prosome, | Hs.464813 | 1E−104 | 91.6667 | 98.63 | Mm.87277 | 2E−89 | 90.182 | 94.178 | down |
| (SEQ ID | | macropain) subunit, alpha type, 8 | | | | | | | | | |
| NO: 1625) | | | | | | | | | | | |
| WAN008ELE_at | PSAT1 | Phosphoserine aminotransferase 1 | Hs.494261 | 7E−27 | 93.1034 | 16.171 | Mm.289936 | 5E−70 | 87.54 | 58.178 | up |
| (SEQ ID | | | | | | | | | | | |
| NO: 1626) | | | | | | | | | | | |
| WAN008EM4_at | ARHGAP18 | Rho GTPase activating protein | Hs.486458 | 1E−109 | 85.3516 | 97.897 | Mm.356496 | 1E−147 | 87.763 | 100 | down |
| (SEQ ID | | 18 | | | | | | | | | |
| NO: 1627) | | | | | | | | | | | |
| WAN008END_at | SCYL1 | SCY1-like 1 (S. cerevisiae) | Hs.238839 | 2E−61 | 83.3333 | 74.844 | Mm.276063 | 0 | 92.292 | 99.792 | down |
| (SEQ ID | | | | | | | | | | | |
| NO: 1628) | | | | | | | | | | | |
| WAN008EOB_at | NOL1 | Nucleolar protein 1, 120 kDa | Hs.534334 | 8E−48 | 89.8058 | 42.474 | Mm.29203 | 1E−120 | 87.248 | 92.165 | up |
| (SEQ ID | | | | | | | | | | | |
| NO: 1629) | | | | | | | | | | | |
| WAN008EQM_at | NA | RC WAN008EQM 11232D-D11 | #N/A | | | | #N/A | | | | down |
| (SEQ ID | | | | | | | | | | | |
| NO: 1630) | | | | | | | | | | | |
| WAN008ERB_at | PCBP1 | Poly(rC) binding protein 1 | Hs.2853 | 0 | 96.7611 | 99.396 | Mm.274146 | 0 | 97.586 | 100 | up |
| (SEQ ID | | | | | | | | | | | |
| NO: 1631) | | | | | | | | | | | |
| WAN008ER1_at | FNBP3 | Formin binding protein 3 | Hs.298735 | 4E−81 | 96.6102 | 98.883 | Mm.257474 | 2E−94 | 99.441 | 100 | down |
| (SEQ ID | | | | | | | | | | | |
| NO: 1632) | | | | | | | | | | | |
| WAN008ERO_at | SNAG1 | Sorting nexin associated golgi | Hs.432755 | 7E−30 | 89.1156 | 28.215 | Mm.33721 | 3E−40 | 90.511 | 26.296 | up |
| (SEQ ID | | protein 1 | | | | | | | | | |
| NO: 1633) | | | | | | | | | | | |
| WAN008ERP_at | LEPREL1 | Leprecan-like 1 | Hs.374191 | 1E−45 | 87.1245 | 92.829 | Mm.326869 | 1E−68 | 88.983 | 94.024 | down |
| (SEQ ID | | | | | | | | | | | |
| NO: 1634) | | | | | | | | | | | |
| WAN008EUO_at | LPL | Lipoprotein lipase | Hs.180878 | 2E−82 | 87.5 | 74.109 | Mm.1514 | 1E−113 | 91.667 | 74.109 | down |
| (SEQ ID | | | | | | | | | | | |
| NO: 1635) | | | | | | | | | | | |
| WAN008EY0_at | C330017I15Rik | RIKEN cDNA C330017I15 gene | Hs.520619 | 1E−179 | 90.2111 | 99.049 | Mm.58660 | 1E−163 | 88.783 | 100 | up |
| (SEQ ID | | | | | | | | | | | |
| NO: 1636) | | | | | | | | | | | |
| WAN008F1P_x_at | NA | WAN008F1P 11165A-A01 | #N/A | | | | #N/A | | | | down |
| (SEQ ID | | | | | | | | | | | |
| NO: 1637) | | | | | | | | | | | |
| WAN013HVJ_at | Rn.75246 | Similar to RIKEN cDNA | #N/A | 7E−78 | 83.6683 | 71.583 | #N/A | 1E−118 | 87.112 | 75.36 | down |
| (SEQ ID | | 2310045A20 | | | | | | | | | |
| NO: 1638) | | | | | | | | | | | |
| WAN013HVL_at | UGDH | UDP-glucose dehydrogenase | Hs.28309 | 1E−160 | 89.3805 | 97.835 | Mm.344831 | 1E−160 | 89.231 | 98.485 | up |
| (SEQ ID | | | | | | | | | | | |
| NO: 1639) | | | | | | | | | | | |
| WAN013HW0_x_at | DQ390542.2 | Mitochondrial NADH | Hs.326475 | 7E−23 | 82.5688 | 16.257 | #N/A | | | | up |
| (SEQ ID | | dehydrogenase subunit 1 | | | | | | | | | |
| NO: 1640) | | | | | | | | | | | |
| WAN013HWB_at | APLP2 | Amyloid beta (A4) precursor-like | Hs.370247 | 3E−08 | 85.5263 | 9.7812 | Mm.19133 | 1E−161 | 95 | 46.332 | down |
| (SEQ ID | | protein 2 | | | | | | | | | |
| NO: 1641) | | | | | | | | | | | |
| WAN013HX8_f_at | EIF4A2 | Eukaryotic translation initiation | Hs.478553 | 1E−155 | 95.8199 | 100 | Mm.260084 | 1E−155 | 95.82 | 100 | down |
| (SEQ ID | | factor 4A, isoform 2 | | | | | | | | | |
| NO: 1490) | | | | | | | | | | | |
| WAN013HZ3_at | ARMCX3 | Armadillo repeat containing, X- | Hs.172788 | 4E−09 | 91.8367 | 9.9796 | Mm.67949 | 4E−29 | 82.684 | 47.047 | down |
| (SEQ ID | | linked 3 | | | | | | | | | |
| NO: 1642) | | | | | | | | | | | |
| WAN013HZK_at | NA | Cluster includes WAN008DS2 | #N/A | | | | #N/A | 7E−10 | 88.372 | 17.587 | up |
| (SEQ ID | | 11228C-H04 | | | | | | | | | |
| NO: 1643) | | | | | | | | | | | |
| WAN013HZP_at | Eif4g2 | Eukaryotic translation initiation | Hs.183684 | 1E−179 | 97.7465 | 72.449 | Mm.185453 | 0 | 99.718 | 72.449 | up |
| (SEQ ID | | factor 4, gamma 2 | | | | | | | | | |
| NO: 1644) | | | | | | | | | | | |
| WAN013I01_at | MCFD2 | Multiple coagulation factor | Hs.293689 | 4E−59 | 89.5604 | 52 | Mm.30251 | 2E−35 | 84.699 | 52.286 | down |
| (SEQ ID | | deficiency 2 | | | | | | | | | |
| NO: 1645) | | | | | | | | | | | |
| WAN013I05_at | Abcb6 | ATP-binding cassette, sub-family | Hs.107911 | 1E−154 | 87.1508 | 100 | Mm.28663 | 0 | 91.806 | 100 | up |
| (SEQ ID | | B (MDR/TAP), member 6 | | | | | | | | | |
| NO: 1646) | | | | | | | | | | | |
| WAN013I15_at | SUCLG2 | Succinate-CoA ligase, GDP- | Hs.186512 | 1E−157 | 85.8195 | 100 | Mm.292637 | 0 | 88.398 | 100 | up |
| (SEQ ID | | forming, beta subunit | | | | | | | | | |
| NO: 1647) | | | | | | | | | | | |
| WAN013I1U_x_at | DQ390542.2 | Mitochondrial NADH | Hs.571926 | 0.00002 | 92.1053 | 7.7079 | #N/A | 0.00001 | 92.105 | 7.7079 | up |
| (SEQ ID | | dehydrogenase subunit 4 | | | | | | | | | |
| NO: 1648) | | | | | | | | | | | |
| WAN013I2F_at | THBD | Thrombomodulin | Hs.2030 | 1E−18 | 88.764 | 18.053 | Mm.24096 | 9E−93 | 85.714 | 92.292 | up |
| (SEQ ID | | | | | | | | | | | |
| NO: 1649) | | | | | | | | | | | |
| WAN013I2K_at | TMEFF1 | Transmembrane protein with | Hs.336224 | 8E−93 | 91.0781 | 100 | Mm.130982 | 7E−86 | 89.963 | 100 | down |
| (SEQ ID | | EGF-like and two follistatin-like | | | | | | | | | |
| NO: 1650) | | domains 1 | | | | | | | | | |
| WAN013I2L_at | SLC7A5 | solute carrier family 7 (cationic | Hs.513797 | 9.00E−07 | 100 | 2.2523 | Mm.27943 | 1.00E−07 | 92 | 3.7538 | up |
| (SEQ ID | | amino acid transporter, y+ | | | | | | | | | |
| NO: 1651) | | system), member 5 | | | | | | | | | |
| WAN013I2T_at | CBX5 | Chromobox homolog 5 (HP1 | Hs.349283 | 1E−142 | 91.8635 | 72.023 | Mm.262059 | 1E−168 | 94.751 | 72.023 | up |
| (SEQ ID | | alpha homolog, Drosophila) | | | | | | | | | |
| NO: 1652) | | | | | | | | | | | |
| WAN013I3P_at | CAMLG | Calcium modulating ligand | Hs.529846 | 1E−147 | 86.7021 | 99.296 | #N/A | 1E−172 | 88.612 | 98.944 | up |
| (SEQ ID | | | | | | | | | | | |
| NO: 1653) | | | | | | | | | | | |
| WAN013I61_at | Nppb | Natriuretic peptide precursor type B | Hs.219140 | | | | Mm.2740 | 5E−30 | 88.281 | 23.146 | down |
| (SEQ ID | | | | | | | | | | | |
| NO: 1654) | | | | | | | | | | | |
| WAN013I6C_at | SLC16A1 | Solute carrier family 16 | Hs.75231 | 2E−26 | 84.472 | 12.697 | Mm.9086 | 1E−110 | 87.24 | 30.284 | up |
| (SEQ ID | | (monocarboxylic acid | | | | | | | | | |
| NO: 1655) | | transporters), member 1 | | | | | | | | | |
| WAN013I6E_x_at | GSTP1 | Glutathione S-transferase pi | Hs.523836 | 1E−129 | 81.9905 | 85.889 | #N/A | 0 | 87.577 | 88.467 | down |
| (SEQ ID | | | | | | | | | | | |
| NO: 1656) | | | | | | | | | | | |
| WAN013I6J_s_at | CAD | Carbamoyl-phosphate synthetase | Hs.377010 | 0 | 91.1552 | 99.461 | Mm.305535 | 0 | 93.502 | 99.461 | up |
| (SEQ ID | | 2, aspartate transcarbamylase, | | | | | | | | | |
| NO: 1657) | | and dihydroorotase | | | | | | | | | |
| WAN013I6P_x_at | ABCB1 | ATP-binding cassette, sub-family | Hs.489033 | 0 | 87.7104 | 28.592 | Mm.146649 | 0 | 89.771 | 33.646 | down |
| (SEQ ID | | B (MDR/TAP), member 1 | | | | | | | | | |
| NO: 1658) | | | | | | | | | | | |
| WAN013I8B_at | Akr1a4 | Aldo-keto reductase family 1, | Hs.474584 | 0 | 91.5371 | 99.314 | Mm.30085 | 0 | 91.71 | 99.314 | down |
| (SEQ ID | | member A4 (aldehyde reductase) | | | | | | | | | |
| NO: 1659) | | | | | | | | | | | |
| WAN013I8V_at | NCL | Nucleolin | Hs.79110 | 1E−111 | 90.0585 | 67.059 | Mm.154378 | 1E−137 | 93.275 | 67.059 | up |
| (SEQ ID | | | | | | | | | | | |
| NO: 1660) | | | | | | | | | | | |
| WAN013I8X_at | HSPD1 | Heat shock 60 kDa protein 1 | Hs.113684 | 0 | 90.308 | 99.775 | Mm.1777 | 0 | 93.388 | 99.775 | Up |
| (SEQ ID | | (chaperonin) | | | | | | | | | |
| NO: 1661) | | | | | | | | | | | |
| WAN013I9F_at | HSPA9B | Heat shock protein 9A | Hs.184233 | 3E−29 | 92.233 | 18.693 | Mm.209419 | 2E−72 | 90.688 | 44.828 | Up |
| (SEQ ID | | | | | | | | | | | |
| NO: 1662) | | | | | | | | | | | |
| WAN013I9G_at | SLC3A2 | Solute carrier family 3 (activators | Hs.502769 | 1E−105 | 84.8544 | 38.064 | Mm.4114 | 0 | 88.596 | 58.98 | Up |
| (SEQ ID | | of dibasic and neutral amino acid | | | | | | | | | |
| NO: 1663) | | transport), member 2 | | | | | | | | | |
| WAN013I9Z_at | GNAS | guanine nucleotide binding | Hs.125898 | | | | Mm.125770 | 0 | 94.403 | 41.104 | Down |
| (SEQ ID | | protein, alpha stimulating | | | | | | | | | |
| NO: 1664) | | | | | | | | | | | |

TABLE 16
High Cell Density Known CHO Sequences

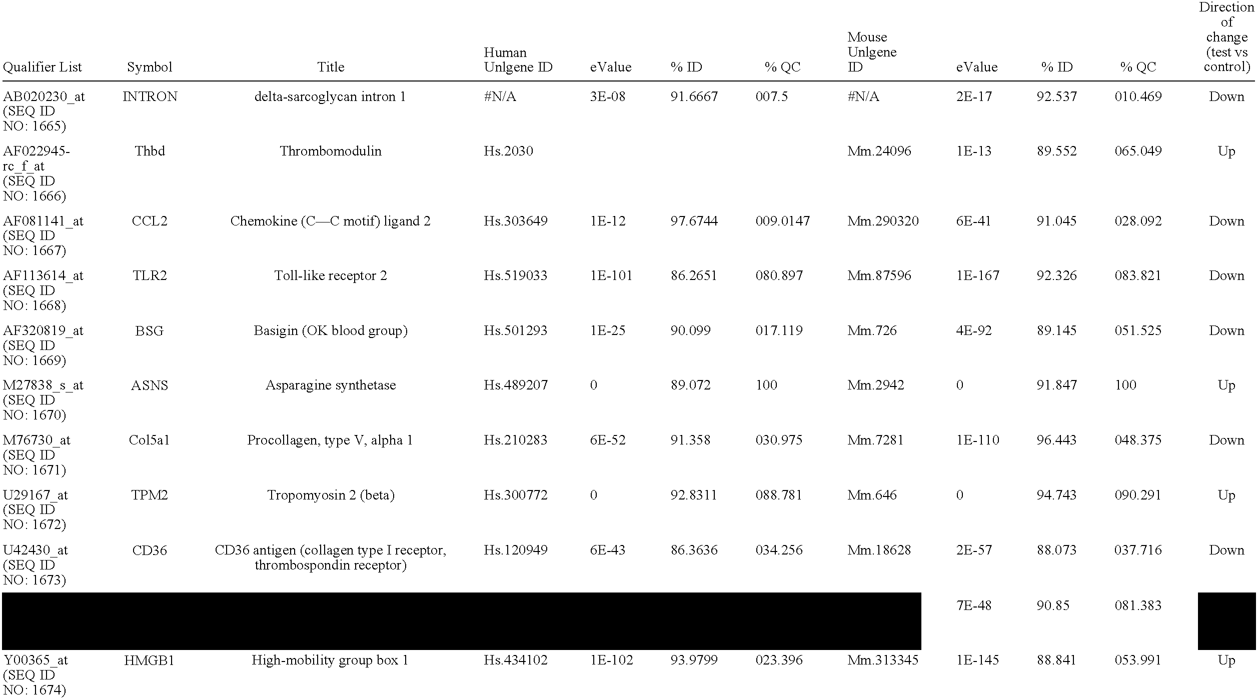

TABLE 17
Control vs. Test HCD4

| | | | | | | | | | | | | |
|---|---|---|---|---|---|---|---|---|---|---|---|---|
| 1.5F Up | | | | | | | | | | | | |
| WAN0088WZ-rc_at | 1.736111 | APC | Adenomatosis polyposis coli | WAN0088WZ- | Hs.158932 | 2E−50 | 84.75177 | 99.29577 | Mm.7883 | 2E−80 | 89.70588 | 95.77465 |
| (SEQ ID NO: 1675) | | | | rc_at Blast | | | | | | | | |
| | | | | Report | | | | | | | | |
| AF052840_x_at | 2.73224 | NA | AF052840 Mesocricetus auratus clone | AF052840_x_at | #N/A | 0 | 0 | 0 | #N/A | 0 | 0 | 0 |
| (SEQ ID NO: 1676) | | | ut3 retroviral-like pol protein (pol) | Blast Report | | | | | | | | |
| | | | mRNA, partial cds. | | | | | | | | | |
| AF306800_at | 1.610306 | NA | AF306800 Cricetulus griseus clone cos7- | AF306800_at | #N/A | 0.00006 | 94.28571 | 2.571639 | #N/A | 0.000008 | 94.44444 | 2.645114 |
| (SEQ ID NO: 1677) | | | 1 intrachromosomal telomeric-like | Blast Report | | | | | | | | |
| | | | sequence. | | | | | | | | | |
| WAN008ED8_at | 1.519757 | AVPI1 | Arginine vasopressin-induced 1 | WAN008ED8_at | Hs.23918 | 3E−44 | 84.27419 | 49.8994 | Mm.30060 | 1E−133 | 88.2716 | 97.78672 |
| (SEQ ID NO: 1678) | | | | Blast Report | | | | | | | | |
| AY011521_x_at | 1.564945 | BMI1 | B lymphoma Mo-MLV insertion region | AY011521_x_at | Hs.496613 | 1E−118 | 96.40288 | 100 | Mm.289584 | 1E−121 | 95.68345 | 100 |
| (SEQ ID NO: 1679) | | | (mouse) | Blast Report | | | | | | | | |
| WAN013I3P_at | 1.745201 | CAMLG | Calcium modulating ligand | WAN013I3P_at | Hs.529846 | 1E−147 | 86.70213 | 99.29577 | #N/A | 1E−172 | 88.6121 | 98.94366 |
| (SEQ ID NO: 1653) | | | | Blast Report | | | | | | | | |
| WAN008CI3_at | 1.811594 | CCNJ | Cyclin J | WAN008CI3_at | Hs.596479 | 1E−145 | 93.71429 | 77.60532 | Mm.309 | 1E−105 | 88.85714 | 77.60532 |
| (SEQ ID NO: 1680) | | | | Blast Report | | | | | | | | |
| D11437_at | 1.506024 | Cyp2c55 | Cytochrome P450, family 2, subfamily c, | D11437_at Blast | #N/A | 2E−73 | 84.0617 | 68.12609 | Mm.142581 | 0 | 91.66667 | 94.57093 |
| (SEQ ID NO: 1681) | | | polypeptide 55 | Report | | | | | | | | |
| X81405_at | 1.587302 | EN2 | Engrailed homolog 2 | X81405_at Blast | Hs.134989 | 5E−69 | 92.59259 | 81.11588 | Mm.4298 | 7E−30 | 92.63158 | 40.77253 |
| (SEQ ID NO: 1682) | | | | Report | | | | | | | | |
| U67146_at | 1.680672 | EEF1E1 | Eukaryotic translation elongation factor 1 | U67146_at Blast | Hs.631818 | 1E−152 | 88.62275 | 63.90306 | Mm.36683 | 0 | 89.70381 | 90.43367 |
| (SEQ ID NO: 1683) | | | epsilon 1 | Report | | | | | | | | |
| WAN008E2Q_at | 3.521127 | GSPT1 | G1 to S phase transition 1 | WAN008E2Q_at | Hs.528780 | 0 | 93.69565 | 100 | Mm.325827 | 0 | 95.86957 | 100 |
| (SEQ ID NO: 1618) | | | | Blast Report | | | | | | | | |
| WAN008CWV_at | 1.66113 | HDGF | Hepatoma-derived growth factor (high- | WAN008CWV_at | Hs.506748 | 3E−75 | 91.48936 | 43.04029 | Mm.292208 | 2E−75 | 91.48936 | 43.04029 |
| (SEQ ID NO: 1684) | | | mobility group protein 1-like) | Blast Report | | | | | | | | |
| U43278_at | 1.626016 | Msr1 | Macrophage scavenger receptor 1 | U43278_at Blast | Hs.632045 | 0 | 0 | 0 | Mm.239291 | 7E−64 | 87.5 | 92.8839 |
| (SEQ ID NO: 1685) | | | | Report | | | | | | | | |
| Z30972_at | 2.09205 | PPARG | Peroxisome proliferative activated | Z30972_at Blast | Hs.162646 | 6E−70 | 91.62562 | 46.88222 | Mm.3020 | 1E−78 | 93.17073 | 47.34411 |
| (SEQ ID NO: 1686) | | | receptor, gamma | Report | | | | | | | | |
| WAN008DRG_at | 1.607717 | PGM1 | Phosphoglucomutase 1 | WAN008DRG_at | Hs.1869 | 1E−164 | 90.88937 | 99.78355 | Mm.2325 | 0 | 93.23144 | 99.1342 |
| (SEQ ID NO: 1687) | | | | Blast Report | | | | | | | | |
| WAN008EWS_at | 1.66113 | PLAA | Phospholipase A2-activating protein | WAN008EWS_at | Hs.27182 | 0 | 93.45133 | 100 | Mm.22724 | 0 | 93.45133 | 100 |
| (SEQ ID NO: 1688) | | | | Blast Report | | | | | | | | |
| WAN008DUZ_at | 1.538462 | POP7 | Processing of precursor 7, ribonuclease | WAN008DUZ_at | Hs.416994 | 6E−52 | 89.50276 | 35.21401 | Mm.290242 | 9E−62 | 91.71271 | 35.21401 |
| (SEQ ID NO: 1689) | | | P subunit (S. cerevisiae) | Blast Report | | | | | | | | |
| WAN0088X2_at | 3.322259 | PEO1 | Progressive external ophthalmoplegia 1 | WAN0088X2_at | Hs.22678 | 1E−141 | 88.65096 | 94.53441 | Mm.105585 | 7E−78 | 90.67797 | 47.77328 |
| (SEQ ID NO: 1593) | | | | Blast Report | | | | | | | | |
| WAN008CWW_at | 2.849003 | PHTF2 | Putative homeodomain transcription | WAN008CWW_at | Hs.203965 | 1E−12 | 86.2069 | 21.75 | Mm.86410 | 1E−17 | 86.86869 | 24.75 |
| (SEQ ID NO: 1690) | | | factor 2 | Blast Report | | | | | | | | |
| WAN008EY3_at | 1.652893 | 5430407P10 | RIKEN cDNA 5430407P10 gene | WAN008EY3_at | #N/A | 0.00005 | 85.48387 | 18.12865 | Mm.133542 | 1E−96 | 88.20059 | 99.12281 |
| (SEQ ID NO: 1691) | | Rik | | Blast Report | | | | | | | | |
| WAN008D6O_at | 1.672241 | Strbp | Spermatid perinuclear RNA binding | WAN008D6O_at | Hs.645506 | 9E−62 | 89.7561 | 67.65677 | Mm.237095 | 3E−94 | 95.34884 | 70.9571 |
| (SEQ ID NO: 1692) | | | protein | Blast Report | | | | | | | | |
| WAN008DXC_at | 2.816901 | TXNDC11 | Thioredoxin domain containing 11 | WAN008DXC_at | Hs.313847 | 0 | 94.0678 | 98.95178 | Mm.291015 | 0 | 94.54927 | 100 |
| (SEQ ID NO: 1693) | | | | Blast Report | | | | | | | | |
| WAN008DWJ_at | 3.636364 | USP1 | Ubiquitin specific peptidase 1 | WAN008DWJ_at | Hs.35086 | 0 | 92.94756 | 97.01754 | Mm.371692 | 0 | 94.90909 | 96.49123 |
| (SEQ ID NO: 1614) | | | | Blast Report | | | | | | | | |
| WAN013HVL_at | 1.760563 | UGDH | UDP-glucose dehydrogenase | WAN013HVL_at | Hs.572518 | 1E−165 | 90.06772 | 95.88745 | Mm.344831 | 1E−158 | 89.01099 | 98.48485 |
| (SEQ ID NO: 1639) | | | | Blast Report | | | | | | | | |
| WAN008CS2_at | 1.569859 | VKORC1L1 | Vitamin K epoxide reductase complex, | WAN008CS2_at | Hs.427232 | 1E−168 | 91.89189 | 96.73203 | Mm.288718 | 0 | 97.28507 | 96.2963 |
| (SEQ ID NO: 1694) | | | subunit 1-like 1 | Blast Report | | | | | | | | |
| X02950_at | 1.547988 | NA | X02950 Hamster alpha-A crystallin gene | X02950_at Blast | #N/A | 0 | 0 | 0 | #N/A | 5E−08 | 86.88525 | 10.87344 |
| (SEQ ID NO: 1695) | | | 5 part (exons 1-3) | Report | | | | | | | | |
| X56207_at | 1.531394 | NA | X56207 Hamster gene for myosin heavy | X56207_at Blast | #N/A | 0 | 0 | 0 | #N/A | 0.00005 | 92.10526 | 6.713781 |
| (SEQ ID NO: 1696) | | | chain, exons 1 & 2 | Report | | | | | | | | |
| WAN008DKJ_x_at | 1.54321 | Zfp297b | Zinc finger protein 297B | WAN008DKJ_x_at | #N/A | 1E−27 | 91.57895 | 90.47619 | Mm.44186 | 2E−24 | 89.69072 | 92.38095 |
| (SEQ ID NO: 1697) | | | | Blast Report | | | | | | | | |
| 1.5F Down | | | | | | | | | | | | |
| AB003732_f_at | 1.542 | NA | AB003732 Cricetulus griseus gene for | AB003732_f_at | #N/A | 2E−14 | 100 | 10.44776 | #N/A | 1E−35 | 88.96104 | 38.30846 |
| (SEQ ID NO: 1698) | | | polyubiquitin, complete cds. | Blast Report | | | | | | | | |
| WAN008EB0_at | 2.179 | ACOT7 | Acyl-CoA thioesterase 7 | WAN008EB0_at | Hs.126137 | 1E−49 | 88.64865 | 50.40872 | Mm.296191 | 3E−70 | 93.04813 | 50.95368 |
| (SEQ ID NO: 1699) | | | | Blast Report | | | | | | | | |
| AF284090_s_at | 1.508 | ADK | Adenosine kinase | AF284090_s_at | Hs.584739 | 2E−42 | 88.41463 | 98.79518 | Mm.188734 | 7E−63 | 93.37349 | 100 |
| (SEQ ID NO: 1700) | | | | Blast Report | | | | | | | | |
| AJ286821_at | 1.629 | NA | AJ286821 Mesocricetus auratus partial | AJ286821_at | #N/A | 0 | 0 | 0 | #N/A | 2E−22 | 85.82677 | 34.23181 |
| (SEQ ID NO: 1701) | | | mRNA for protein tyrosine phosphatase | Blast Report | | | | | | | | |
| | | | (ptp gene) | | | | | | | | | |
| WAN013I8B_at | 1.542 | AKR1A1 | Aldo-keto reductase family 1, member A1 | WAN013I8B_at | Hs.474584 | 1E−170 | 87.56567 | 97.94168 | #N/A | 0 | 91.53713 | 99.31389 |
| (SEQ ID NO: 1659) | | | (aldehyde reductase) | Blast Report | | | | | | | | |
| WAN013IAG_at | 6.5 | Areg | Amphiregulin | WAN013IAG_at | Hs.270833 | 2E−14 | 84.21053 | 25 | Mm.8039 | 3E−49 | 89.20455 | 38.59649 |
| (SEQ ID NO: 1702) | | | | Blast Report | | | | | | | | |
| WAN008DGD_at | 1.61 | Aplp2 | Amyloid beta (A4) precursor-like | WAN008DGD_at | Hs.370247 | 0 | 0 | 0 | Mm.19133 | 6E−69 | 93.08756 | 44.46721 |
| (SEQ ID NO: 1564) | | | protein 2 | Blast Report | | | | | | | | |
| WAN008EMP_at | 1.709 | CAP1 | CAP, adenylate cyclase-associated | WAN008EMP_at | Hs.370581 | 1E−56 | 93.40659 | 35.20309 | Mm.8687 | 1E−88 | 92.01389 | 55.706 |
| (SEQ ID NO: 1703) | | | protein 1 (yeast) | Blast Report | | | | | | | | |
| WAN013I0Y_at | 1.523 | CAPG | Capping protein (actin filament), gelsolin- | WAN013I0Y_at | Hs.516155 | 1E−112 | 87.1134 | 72.52336 | Mm.18626 | 1E−153 | 89.65517 | 81.30841 |
| (SEQ ID NO: 1704) | | | like | Blast Report | | | | | | | | |
| AF081141_at | 21.04 | CCL2 | Chemokine (C-C motif) ligand 2 | AF081141_at | Hs.303649 | 3E−13 | 90.625 | 13.41719 | Mm.290320 | 5E−41 | 91.04478 | 28.09224 |
| (SEQ ID NO: 1667) | | | | Blast Report | | | | | | | | |
| WAN013I8F_at | 2.19 | NA | Cluster includes AF308456 Cricetulus | WAN013I8F_at | #N/A | 0 | 0 | 0 | #N/A | 4E−07 | 91.80328 | 4.552239 |
| (SEQ ID NO: 1705) | | | griseus intracellular adhesion molecule 1 | Blast Report | | | | | | | | |
| | | | (ICAM1) mRNA, complete cds. | | | | | | | | | |
| WAN013I8Y_at | 1.528 | NA | Cluster includes M23159 Chinese | WAN013I8Y_at | #N/A | 0 | 84.15233 | 70.65972 | #N/A | 0 | 89.18919 | 70.65972 |
| (SEQ ID NO: 1706) | | | hamster DHFR-coamplified protein | Blast Report | | | | | | | | |
| | | | mRNA, partial cds, clone 2BE2121. | | | | | | | | | |
| WAN013HWP_x_at | 1.59 | NA | Cluster includes WAN008CUN 10602C- | WAN013HWP_x_at | #N/A | 0 | 0 | 0 | #N/A | 8E−76 | 92.82297 | 45.93407 |
| (SEQ ID NO: 1707) | | | E01 | Blast Report | | | | | | | | |
| WAN013HZK_at | 2.279 | NA | Cluster includes WAN008DS2 11228C- | WAN013HZK_at | #N/A | 0 | 0 | 0 | #N/A | 6E−10 | 88.37209 | 17.58691 |
| (SEQ ID NO: 1643) | | | H04 | Blast Report | | | | | | | | |
| WAN013I73_at | 1.564 | NA | Cluster includes X61958 C. longicaudatus | WAN013I73_at | #N/A | 0 | 0 | 0 | #N/A | 4E−45 | 88.54167 | 42.01313 |
| (SEQ ID NO: 1708) | | | mRNA for thrombin receptor | Blast Report | | | | | | | | |
| WAN008EHM_at | 2.033 | CLU | Clusterin | WAN008EHM_at | Hs.436657 | 1E−71 | 91.38756 | 36.60245 | Mm.200608 | 1E−91 | 90.74074 | 47.28546 |
| (SEQ ID NO: 1709) | | | | Blast Report | | | | | | | | |
| WAN013HWY_at | 1.658 | CCDC80 | Coiled-coil domain containing 80 | WAN013HWY_at | Hs.477128 | 6E−92 | 86.55914 | 76.07362 | Mm.181074 | 1E−171 | 90.57377 | 99.7955 |
| (SEQ ID NO: 1710) | | | | Blast Report | | | | | | | | |
| WAN008CST_at | 1.581 | COPS2 | COP9 constitutive photomorphogenic | WAN008CST_at | Hs.369614 | 0 | 94.16058 | 100 | Mm.3596 | 0 | 96.89781 | 100 |
| (SEQ ID NO: 1711) | | | homolog subunit 2 (Arabidopsis) | Blast Report | | | | | | | | |
| U71399_at | 1.593 | Cyb5 | Cytochrome b-5 | U71399_at Blast | #N/A | 4E−62 | 85.09934 | 60.4 | Mm.31018 | 1E−103 | 90.9396 | 59.6 |
| (SEQ ID NO: 1712) | | | | Report | | | | | | | | |
| WAN0088LE_at | 1.52 | DSTN | Destrin (actin depolymerizing factor) | WAN0088LE_at | Hs.304192 | 1E−169 | 90.38855 | 87.47764 | Mm.28919 | 0 | 93.04511 | 95.16995 |
| (SEQ ID NO: 1713) | | | | Blast Report | | | | | | | | |
| WAN008EAZ_at | 1.597 | DPP3 | Dipeptidyl-peptidase 3 | WAN008EAZ_at | Hs.502914 | 1E−161 | 90.9292 | 96.17021 | Mm.234769 | 0 | 94.68085 | 100 |
| (SEQ ID NO: 1714) | | | | Blast Report | | | | | | | | |
| WAN008CYZ_at | 1.929 | EFNB2 | Ephrin-B2 | WAN008CYZ_at | Hs.149239 | 2E−27 | 85.81081 | 25.69444 | Mm.209813 | 1E−102 | 89.6648 | 62.15278 |
| (SEQ ID NO: 1715) | | | | Blast Report | | | | | | | | |
| WAN013HYK_at | 3.18 | EPS8 | Epidermal growth factor receptor | WAN013HYK_at | Hs.591160 | 2E−15 | 89.28571 | 14.50777 | Mm.235346 | 2E−13 | 93.75 | 13.81693 |
| (SEQ ID NO: 1716) | | | pathway substrate 8 | Blast Report | | | | | | | | |
| AF046870_at | 1.536 | Efemp2 | Epidermal growth factor-containing | AF046870_at | #N/A | 0 | 88.50856 | 92.4642 | Mm.276367 | 0 | 91.79567 | 97.36247 |
| (SEQ ID NO: 1717) | | | fibulin-like extracellular matrix protein 2 | Blast Report | | | | | | | | |
| WAN013I3F_at | 1.816 | ETHE1 | Ethylmalonic encephalopathy 1 | WAN013I3F_at | Hs.7486 | 1E−111 | 84.33515 | 98.21109 | Mm.29553 | 0 | 90.87657 | 100 |
| (SEQ ID NO: 1718) | | | | Blast Report | | | | | | | | |
| WAN008EUG_at | 1.621 | EXOC6 | Exocyst complex component 6 | WAN008EUG_at | Hs.292097 | 2E−97 | 89.27445 | 96.06061 | Mm.24865 | 1E−124 | 93.44262 | 92.42424 |
| (SEQ ID NO: 1719) | | | | Blast Report | | | | | | | | |
| AF061256_at | 1.971 | FOLR1 | Folate receptor 1 (adult) | AF061256_at | Hs.73769 | 1E−124 | 83.96825 | 61.76471 | Mm.2135 | 0 | 89.23077 | 76.47059 |
| (SEQ ID NO: 1720) | | | | Blast Report | | | | | | | | |
| WAN008EMJ_at | 1.873 | GPC6 | Glypican 6 | WAN008EMJ_at | Hs.444329 | 7E−64 | 92.55319 | 37.4502 | Mm.234129 | 3E−58 | 90.37433 | 37.251 |
| (SEQ ID NO: 1721) | | | | Blast Report | | | | | | | | |
| WAN0088O4_at | 1.556 | IMPDH1 | IMP (inosine monophosphate) | WAN0088O4_at | Hs.534808 | 1E−170 | 91.64835 | 89.39096 | Mm.260707 | 0 | 91.28713 | 99.21415 |
| (SEQ ID NO: 1722) | | | dehydrogenase 1 | Blast Report | | | | | | | | |
| WAN013I3K_at | 1.872 | IDH1 | Isocitrate dehydrogenase 1 (NADP+), | WAN013I3K_at | Hs.11223 | 0 | 91.04478 | 99.44341 | Mm.9925 | 0 | 93.47015 | 99.44341 |
| (SEQ ID NO: 1723) | | | soluble | Blast Report | | | | | | | | |
| WAN008E63_at | 1.733 | JAK1 | Janus kinase 1 (a protein tyrosine kinase) | WAN008E63_at | Hs.207538 | 1E−142 | 89.83834 | 82.79159 | Mm.289657 | 0 | 94.53303 | 83.93881 |
| (SEQ ID NO: 1724) | | | | Blast Report | | | | | | | | |
| WAN008E9N_at | 1.542 | KLHL7 | Kelch-like 7 (Drosophila) | WAN008E9N_at | Hs.385861 | 1E−150 | 89.44223 | 99.40594 | #N/A | 0 | 93.11111 | 89.10891 |
| (SEQ ID NO: 1620) | | | | Blast Report | | | | | | | | |
| WAN008EZV_at | 1.777 | KIF1B | Kinesin family member 1B | WAN008EZV_at | Hs.97858 | 1E−176 | 89.38053 | 98.77622 | Mm.402393 | 1E−14 | 89.18919 | 12.93706 |
| (SEQ ID NO: 1725) | | | | Blast Report | | | | | | | | |
| AF093673_at | 1.703 | LLN | layilin | AF093673_at | #N/A | 1E−158 | 88.54962 | 38.67159 | #N/A | 1E−149 | 90.2439 | 33.28413 |
| (SEQ ID NO: 1726) | | | | Blast Report | | | | | | | | |
| M96676_at | 1.542 | LGALS1 | Lectin, galactoside-binding, soluble, 1 | M96676_at Blast | Hs.445351 | 1E−122 | 88.94472 | 100 | Mm.43831 | 1E−131 | 89.94975 | 100 |
| (SEQ ID NO: 1727) | | | (galectin 1) | Report | | | | | | | | |
| WAN008ERP_at | 1.673 | LEPREL1 | Leprecan-like 1 | WAN008ERP_at | Hs.374191 | 1E−45 | 87.12446 | 92.82869 | Mm.326869 | 1E−68 | 88.98305 | 94.0239 |
| (SEQ ID NO: 1634) | | | | Blast Report | | | | | | | | |
| WAN008EUO_at | 2.105 | LPL | Lipoprotein lipase | WAN008EUO_at | Hs.180878 | 2E−82 | 87.5 | 74.10926 | Mm.1514 | 1E−114 | 91.66667 | 74.10926 |
| (SEQ ID NO: 1635) | | | | Blast Report | | | | | | | | |
| L18986_at | 1.619 | LAMP1 | Lysosomal-associated membrane protein 1 | L18986_at Blast | Hs.494419 | 4E−58 | 85.66176 | 20.76336 | Mm.16716 | 1E−157 | 86.94915 | 45.03817 |
| (SEQ ID NO: 1501) | | | | Report | | | | | | | | |
| WAN013I8P_at | 1.818 | LAMP2 | Lysosomal-associated membrane protein 2 | WAN013I8P_at | Hs.496684 | 1E−133 | 86.23853 | 93.32192 | Mm.486 | 0 | 91.20287 | 95.37671 |
| (SEQ ID NO: 1728) | | | | Blast Report | | | | | | | | |
| AF306662_at | 2.002 | MMP14 | Matrix metallopeptidase 14 (membrane- | AF306662_at | Hs.2399 | 0 | 89.72603 | 99.82906 | Mm.280175 | 0 | 90.08547 | 100 |
| (SEQ ID NO: 1729) | | | inserted) | Blast Report | | | | | | | | |
| WAN008DBL_at | 1.568 | NDUFB9 | NADH dehydrogenase (ubiquinone) 1 | WAN008DBL_at | Hs.15977 | 1E−120 | 85.76923 | 93.86282 | Mm.322294 | 1E−180 | 90.67961 | 92.96029 |
| (SEQ ID NO: 1730) | | | beta subcomplex, 9, 22 kDa | Blast Report | | | | | | | | |
| WAN008BSC_at | 1.706 | NRD1 | Nardilysin (N-arginine dibasic | WAN008BSC_at | Hs.584782 | 1E−113 | 90.82569 | 60.78067 | Mm.274950 | 1E−131 | 93.00912 | 61.15242 |
| (SEQ ID NO: 1731) | | | convertase) | Blast Report | | | | | | | | |
| WAN013I62_at | 1.607 | ODC1 | Ornithine decarboxylase 1 | WAN013I62_at | Hs.467701 | 1E−178 | 85.99222 | 56.64952 | Mm.34102 | 0 | 91.76788 | 54.44526 |
| (SEQ ID NO: 1732) | | | | Blast Report | | | | | | | | |
| WAN008DXO_at | 2.241 | OSBPL9 | Oxysterol binding protein-like 9 | WAN008DXO_at | Hs.21938 | 0 | 92.9368 | 98.89706 | Mm.366315 | 0 | 93.93382 | 100 |
| (SEQ ID NO: 1733) | | | | Blast Report | | | | | | | | |
| AB014875_at | 1.787 | PLS3 | plastin 3 (T isoform) | AB014875_at | Hs.496622 | 1E−159 | 92 | 31.59851 | Mm.28777 | 1E−175 | 90.32847 | 40.74349 |
| (SEQ ID NO: 1734) | | | | Blast Report | | | | | | | | |
| AF221841_at | 1.564 | Mm.379870 | PREDICTED: Mus musculus similar to | AF221841_at | #N/A | 0 | 90.97889 | 100 | #N/A | 0 | 93.56725 | 98.46449 |
| (SEQ ID NO: 1735) | | | Peroxiredoxin 1 (Thioredoxin peroxidase | Blast Report | | | | | | | | |
| | | | 2) (Thioredoxin-dependent peroxide | | | | | | | | | |
| | | | reductase 2) (Osteoblast specific factor | | | | | | | | | |
| | | | 3) (OSF-3) (Macrophage 23 kDa stress | | | | | | | | | |
| | | | protein) (LOC545161), mRNA | | | | | | | | | |
| WAN008DSZ_at | 1.522 | PPP1R7 | Protein phosphatase 1, regulatory | WAN008DSZ_at | Hs.36587 | 1E−125 | 89.08189 | 100 | Mm.88704 | 1E−163 | 93.05211 | 100 |
| (SEQ ID NO: 1736) | | | subunit 7 | Blast Report | | | | | | | | |
| AB056121_at | 2.595 | S100B | S100 calcium binding protein, beta | AB056121_at | Hs.422181 | 6E−85 | 89.28571 | 83.58209 | Mm.235998 | 1E−108 | 90.14925 | 100 |
| (SEQ ID NO: 1737) | | | (neural) | Blast Report | | | | | | | | |
| WAN008DSW_at | 1.732 | STAT3 | Signal transducer and activator of | WAN008DSW_at | Hs.463059 | 0 | 91.91729 | 97.25777 | Mm.249934 | 0 | 94.11765 | 99.45155 |
| (SEQ ID NO: 1738) | | | transcription 3 (acute-phase response | Blast Report | | | | | | | | |
| | | | factor) | | | | | | | | | |
| WAN013I2Q_at | 1.694 | SSU72 | SSU72 RNA polymerase II CTD | WAN013I2Q_at | Hs.30026 | 1E−132 | 91.57303 | 71.91919 | Mm.294770 | 1E−171 | 91.47982 | 90.10101 |
| (SEQ ID NO: 1739) | | | phosphatase homolog (S. cerevisiae) | Blast Report | | | | | | | | |
| WAN008D44_at | 1.854 | SCP2 | Sterol carrier protein 2 | WAN008D44_at | Hs.476365 | 1E−115 | 89.53168 | 64.3617 | Mm.379011 | 1E−132 | 91.46006 | 64.3617 |
| (SEQ ID NO: 1740) | | | | Blast Report | | | | | | | | |
| WAN008EKU_at | 1.503 | TAX1BP1 | Tax1 (human T-cell leukemia virus type I) | WAN008EKU_at | Hs.34576 | 1E−120 | 89.5122 | 99.51456 | Mm.431979 | 0 | 0 | 0 |
| (SEQ ID NO: 1741) | | | binding protein 1 | Blast Report | | | | | | | | |
| WAN013I4X-at | 1.676 | Timp2 | Tissue inhibitor of metalloproteinase 2 | WAN013I4X_at | Hs.633514 | 0 | 0 | 0 | Mm.206505 | 0 | 94.2623 | 100 |
| (SEQ ID NO: 1742) | | | | Blast Report | | | | | | | | |
| AF113614_at | 1.823 | TLR2 | Toll-like receptor 2 | AF113614_at | Hs.519033 | 1E−101 | 86.26506 | 80.89669 | Mm.87596 | 1E−167 | 92.32558 | 83.82066 |
| (SEQ ID NO: 1668) | | | | Blast Report | | | | | | | | |
| WAN008DM2_at | 1.541 | TRAPPC3 | Trafficking protein particle complex 3 | WAN008DM2_at | Hs.523131 | 1E−129 | 93.18885 | 69.3133 | Mm.8392 | 1E−130 | 93.18885 | 69.3133 |
| (SEQ ID NO: 1743) | | | | Blast Report | | | | | | | | |
| WAN013I8T_at | 1.877 | Mm.392113 | Transcribed locus, moderately similar to | WAN013I8T_at | #N/A | 0 | 90.74941 | 79.66418 | #N/A | 0 | 94.36202 | 94.3097 |
| (SEQ ID NO: 1744) | | | XP_426592.1 PREDICTED: similar to | Blast Report | | | | | | | | |
| | | | tubulin, alpha 2; tubulin alpha 2 [Gallus | | | | | | | | | |
| | | | gallus] | | | | | | | | | |
| WAN0088TW_at | 1.618 | TCEB3 | Transcription elongation factor B (SIII), | WAN0088TW_at | Hs.584806 | 1E−173 | 89.36567 | 98.52941 | Mm.27663 | 0 | 93.09701 | 98.52941 |
| (SEQ ID NO: 1745) | | | polypeptide 3 (110 kDa, elongin A) | Blast Report | | | | | | | | |
| WAN008CZR_at | 1.522 | TMED1 | Transmembrane emp24 protein transport | WAN008CZR_at | Hs.515139 | 1E−139 | 87.57515 | 90.72727 | Mm.196618 | 0 | 94.89194 | 92.54545 |
| (SEQ ID NO: 1746) | | | domain containing 1 | Blast Report | | | | | | | | |
| WAN008D2L_at | 2.049 | TMEM50A | Transmembrane protein 50A | WAN008D2L_at | Hs.523054 | 2E−76 | 85.79882 | 80.09479 | Mm.88349 | 1E−63 | 86.92308 | 61.61137 |
| (SEQ ID NO: 1747) | | | | Blast Report | | | | | | | | |
| WAN013I4D_at | 2.02 | TAP2 | Transporter 2, ATP-binding cassette, | WAN013I4D_at | Hs.502 | 5E−33 | 83.25792 | 46.33124 | Mm.14814 | 1E−111 | 88.95028 | 75.89099 |
| (SEQ ID NO: 1532) | | | sub-family B (MDR/TAP) | Blast Report | | | | | | | | |
| U22818_at | 1.582 | NA | U22818 Cricetulus griseus SRD-2 mutant | U22818_at Blast | #N/A | 0 | 0 | 0 | #N/A | 1E−35 | 87.81726 | 15.91276 |
| (SEQ ID NO: 1748) | | | sterol regulatory element binding protein- | Report | | | | | | | | |
| | | | 2 (SREBP-2) mRNA, complete cds. | | | | | | | | | |
| AF004368_at | 1.618 | UGP2 | UDP-glucose pyrophosphorylase 2 | AF004368_at | Hs.516217 | 1E−116 | 91.77215 | 53.92491 | Mm.28877 | 1E−147 | 90.86538 | 70.98976 |
| (SEQ ID NO: 1749) | | | | Blast Report | | | | | | | | |
| WAN008D7G_at | 1.52 | VAMP3 | Vesicle-associated membrane protein 3 | WAN008D7G_at | Hs.66708 | 4E−07 | 97.56098 | 7.400722 | Mm.273930 | 9E−19 | 91.86047 | 15.52347 |
| (SEQ ID NO: 1750) | | | (cellubrevin) | Blast Report | | | | | | | | |
| WAN013HVO_at | 1.541 | YBX1 | Y box binding protein 1 | WAN013HVO_at | Hs.473583 | 0 | 93.86139 | 99.40945 | Mm.258204 | 0 | 95.54455 | 99.40945 |
| (SEQ ID NO: 1751) | | | | Blast Report | | | | | | | | |

### Example 9

### Genes Differentially Expressed in Cells with Sustained High Cell Viability

Bcl-xL is a powerful inhibitor of cell death. Cells overpressing Bcl-xL demonstrate sustained high cell viability. Tables 18 and 19 summarize nucleic acids that are differentially expressed by a factor of at least 1.2 in cells overexpressing Bcl-xL. Samples were taken at multiple time points for comparison. Table 18 summarizes nucleic acids that are differentially expressed by a factor of at least 1.2 at day 5. Table 19 summarizes nucleic acids that are differentially expressed by a factor of at least 1.2 at a stage later than day 5.

TABLE 18
d5 comparison

| | Fold | | | | |
|---|---|---|---|---|---|
| Qualifier List | Change | Symbol | Title | Human Unigene ID | eValue |
| DOWN (Originally | | | | | |
| 173) | | | | | |
| WAN008DUG_at | 1.582 | Hibadh | 3-hydroxyisobutyrate | WAN008DUG_at Blast | Hs.406758 |
| (SEQ ID NO: 1752) | | | dehydrogenase | Report | |
| WAN008EB0_at | 1.796 | ACOT7 | Acyl-CoA thioesterase 7 | WAN008EB0_at Blast | Hs.126137 |
| (SEQ ID NO: 1699) | | | | Report | |
| WAN008CT8_at | 1.533 | AP2M1 | Adaptor-related protein | WAN008CT8_at Blast | Hs.518460 |
| (SEQ ID NO: 1522) | | | complex 2, mu 1 subunit | Report | |
| AF120325_x_at | 2.101 | NA | AF120325 Cricetutus | AF120325_x_at Blast | #N/A |
| (SEQ ID NO: 1753) | | | griseus class I beta tubulin | Report | |
| | | | gene, complete cds. | | |
| WAN008EKP_at | 1.752 | ATP5B | ATP synthase, H+ | WAN008EKP_at Blast | Hs.406510 |
| (SEQ ID NO: 1754) | | | transporting, mitochondrial | Report | |
| | | | F1 complex, beta | | |
| | | | polypeptide | | |
| WAN008F20_x_at | 1.544 | BCCIP | BRCA2 and CDKN1A | WAN008F20_x_at Blast | Hs.370292 |
| (SEQ ID NO: 1755) | | | interacting protein | Report | |
| WAN008DPO_at | 1.638 | BTBD1 | BTB (POZ) domain | WAN008DPO_at Blast | Hs.459149 |
| (SEQ ID NO: 1756) | | | containing 1 | Report | |
| WAN013I3P_at | 1.804 | CAMLG | Calcium modulating ligand | WAN01313P_at Blast | Hs.529846 |
| (SEQ ID NO: 1653) | | | Report | | |
| WAN008DSX_at | 3.055 | CALM1 | Calmodulin 1 | WAN008DSX_at Blast | Hs.282410 |
| (SEQ ID NO: 1757) | | | (phosphorylase kinase, | Report | |
| | | | delta) | | |
| WAN013HVK_at | 1.581 | Arpp19 | CAMP-regulated | WAN013HVK_at Blast | #N/A |
| (SEQ ID NO: 1758) | | | phosphoprotein 19 | Report | |
| WAN008ECX_at | 1.628 | Cd151 | CD151 antigen | WAN008ECX_at Blast | #N/A |
| (SEQ ID NO: 1759) | | | | Report | |
| WAN008D27_at | 1.57 | CLTA | Clathrin, light polypeptide | WAN008D27_at Blast | Hs.522114 |
| (SEQ ID NO: 1760) | | | (Lca) | Report | |
| WAN008DS9_at | 1.583 | CFL2 | Cofilin 2 (muscle) | WAN008DS9_at Blast | Hs.180141 |
| (SEQ ID NO: 1613) | | | | Report | |
| AF022941_x_at | 2.041 | Cirbp | Cold inducible RNA binding | AF022941_x_at Blast | Hs.634522 |
| (SEQ ID NO: 1761) | | | protein | Report | |
| WAN008EEB_at | 1.779 | CORO1B | Coronin, actin binding | WAN008EEB_at Blast | Hs.6191 |
| (SEQ ID NO: 1762) | | | protein, 1B | Report | |
| WAN0088J2_at | 1.63 | CUEDC2 | CUE domain containing 2 | WAN0088J2_at Blast | Hs.500874 |
| (SEQ ID NO: 1763) | | | | Report | |
| WAN0088PY_at | 1.609 | Ddx5 | DEAD (Asp-Glu-Ala-Asp) | WAN0088PY_at Blast | Hs.279806 |
| (SEQ ID NO: 1480) | | | box polypeptide 5 | Report | |
| WAN013I1H_at | 1.601 | D19Bwg1357e | DNA segment Chr 19, | WAN013I1H_at Blast | #N/A |
| (SEQ ID NO: 1764) | | | Brigham & Women's | Report | |
| | | | Genetics 1357 expressed | | |
| WAN008DVF_at | 1.509 | DNAJC7 | DnaJ (Hsp40) homolog, | WAN008DVF_at Blast | Hs.500156 |
| (SEQ ID NO: 1765) | | | subfamily C, member 7 | Report | |
| WAN013I3F_at | 1.544 | ETHE1 | Ethylmalonic | WAN013I3F_at Blast | Hs.7486 |
| (SEQ ID NO: 1718) | | | encephalopathy 1 | Report | |
| WAN013HZ5_at | 1.592 | EIF2S2 | Eukaryotic translation | WAN013HZ5_at Blast | Hs.429180 |
| (SEQ ID NO: 1766) | | | initiation factor 2, subunit 2 | Report | |
| | | | beta, 38 kDa | | |
| WAN008E8R_at | 1.522 | EIF3S1 | Eukaryotic translation | WAN008E8R_at Blast | Hs.404056 |
| (SEQ ID NO: 1767) | | | initiation factor 3, subunit 1 | Report | |
| | | | alpha, 35 kDa | | |
| WAN013HZP_at | 1.532 | Eif4g2 | Eukaryotic translation | WAN013HZP_at Blast | Hs.183684 |
| (SEQ ID NO: 1644) | | | initiation factor 4, gamma 2 | Report | |
| WAN013IA4_at | 1.97 | ETF1 | Eukaryotic translation | WAN013IA4_at Blast | Hs.483494 |
| (SEQ ID NO: 1768) | | | termination factor 1 | Report | |
| WAN008E82_at | 1.522 | Fbxl11 | F-box and leucine-rich | WAN008E82_at Blast | Hs.124147 |
| (SEQ ID NO: 1769) | | | repeat protein 11 | Report | |
| D43757_at | 1.581 | FGA | Fibrinogen alpha chain | D43757_at Blast Report | Hs.351593 |
| (SEQ ID NO: 1770) | | | | | |
| WAN008E72_x_at | 2.339 | GDI2 | GDP dissociation inhibitor 2 | WAN008E72_x_at Blast | Hs.299055 |
| (SEQ ID NO: 1547) | | | | Report | |
| WAN008EXR_at | 1.574 | GPI | Glucose phosphate | WAN008EXR_at Blast | Hs.466471 |
| (SEQ ID NO: 1771) | | | isomerase | Report | |
| WAN008EQH_at | 1.813 | GLUD1 | Glutamate dehydrogenase 1 | WAN008EQH_at Blast | Hs.500409 |
| (SEQ ID NO: 1772) | | | | Report | |
| WAN008BR0_at | 1.564 | GOT2 | Glutamic-oxaloacetic | WAN008BR0_at Blast | Hs.599470 |
| (SEQ ID NO: 1773) | | | transaminase 2, | Report | |
| | | | mitochondrial (aspartate | | |
| | | | aminotransferase 2) | | |
| WAN008CFZ_at | 1.601 | HSBP1 | Heat shock factor binding | WAN008CFZ_at Blast | Hs.250899 |
| (SEQ ID NO: 1774) | | | protein 1 | Report | |
| WAN013I1P_at | 1.587 | HNRPA2B1 | Heterogeneous nuclear | WAN013I1P_at Blast | Hs.487774 |
| (SEQ ID NO: 1492) | | | ribonucleoprotein A2/B1 | Report | |
| Y00365_at | 1.895 | HMGB1 | High-mobility group box 1 | Y00365_at Blast Report | Hs.434102 |
| (SEQ ID NO: 1674) | | | | | |
| WAN008EKL_at | 1.512 | HBP1 | HMG-box transcription | WAN008EKL_at Blast | Hs.162032 |
| (SEQ ID NO: 1775) | | | factor 1 | Report | |
| WAN013I8N_at | 1.709 | IMPDH2 | IMP (inosine | WAN013I8N_at Blast | Hs.476231 |
| (SEQ ID NO: 1776) | | | monophosphate) | Report | |
| | | | dehydrogenase 2 | | |
| WAN0088O9_at | 2.845 | Itgb1 | Integrin beta 1 (fibronectin | WAN0088O9_at Blast | Hs.295626 |
| (SEQ ID NO: 1777) | | | receptor beta) | Report | |
| AF180918_at | 1.742 | KLHL5 | Kelch-like 5 (Drosophila) | AF180918_at Blast Report | Hs.272251 |
| (SEQ ID NO: 1778) | | | | | |
| WAN008E26_x_at | 2.466 | KLHL7 | Kelch-like 7 (Drosophila) | WAN008E26_x_at Blast | Hs.385861 |
| (SEQ ID NO: 1779) | | | | Report | |
| WAN008BNG_at | 1.568 | LRRC28 | Leucine rich repeat | WAN008BNG_at Blast | Hs.578684 |
| (SEQ ID NO: 1780) | | | containing 28 | Report | |
| WAN008EKF_at | 1.623 | Lass2 | Longevity assurance | WAN008EKF_at Blast | Hs.643565 |
| (SEQ ID NO: 1781) | | | homolog 2 (S. cerevisiae) | Report | |
| L18986_at | 1.584 | LAMP1 | Lysosomal-associated | L18986_at Blast Report | Hs.494419 |
| (SEQ ID NO: 1501) | | | membrane protein 1 | | |
| WAN008F1L_at | 1.676 | Mxi1 | Max interacting protein 1 | WAN008F1L_at Blast | Hs.501023 |
| (SEQ ID NO: 1782) | | | | Report | |
| WAN008ESO_at | 1.757 | MPP6 | Membrane protein, | WAN008ESO_at Blast | Hs.533355 |
| (SEQ ID NO: 1783) | | | palmitoylated 6 (MAGUK | Report | |
| | | | P55 subfamily member 6) | | |
| J00061_at | 1.861 | MT1 | metallothionein I | J00061_at Blast Report | #N/A |
| (SEQ ID NO: 1784) | | | | | |
| X79864_at | 1.741 | MRPL12 | Mitochondrial ribosomal | X79864_at Blast Report | Hs.109059 |
| (SEQ ID NO: 1785) | | | protein L12 | | |
| WAN008E43_at | 1.583 | MRPL30 | Mitochondrial ribosomal | WAN008E43_at Blast | Hs.590896 |
| (SEQ ID NO: 1786) | | | protein L30 | Report | |
| WAN008DKS_at | 1.615 | MAPK8IP1 | Mitogen-activated protein | WAN008DKS_at Blast | Hs.234249 |
| (SEQ ID NO: 1787) | | | kinase 8 interacting protein 1 | Report | |
| WAN008CQE_at | 1.747 | NDUFB6 | NADH dehydrogenase | WAN008CQE_at Blast | Hs.493668 |
| (SEQ ID NO: 1788) | | | (ubiquinone) 1 beta | Report | |
| | | | subcomplex, 6, 17 kDa | | |
| WAN008EE0_x_at | 1.671 | NDUFS1 | NADH dehydrogenase | WAN008EE0_x_at Blast | Hs.471207 |
| (SEQ ID NO: 1789) | | | (ubiquinone) Fe—S protein 1, | Report | |
| | | | 75 kDa (NADH-coenzyme Q | | |
| | | | redactase) | | |
| WAN013I17_at | 2.237 | NID1 | Nidogen 1 | WAN013I17_at Blast | Hs.356624 |
| (SEQ ID NO: 1790) | | | | Report | |
| WAN008BNY_at | 1.61 | NSMCE1 | Non-SMC element 1 | WAN008BNY_at Blast | Hs.284295 |
| (SEQ ID NO: 1791) | | | homolog (S. cerevisiae) | Report | |
| WAN0088KK_x_at | 1.779 | Nfe2I2 | Nuclear factor, erythroid | WAN0088KK_x_at Blast | Hs.155396 |
| (SEQ ID NO: 1792) | | | derived 2, like 2 | Report | |
| WAN013I62_at | 1.843 | ODC1 | Ornithine decarboxylase 1 | WAN013I62_at Blast | Hs.467701 |
| (SEQ ID NO: 1732) | | | | Report | |
| WAN013I2X_at | 2.366 | PPIG | Peptidylprolyl isomerase G | WAN013I2X_at Blast | Hs.470544 |
| (SEQ ID NO: 1793) | | | (cyclophilin G) | Report | |
| AB041733_at | 1.693 | PEX12 | Peroxisomal biogenesis | AB041733_at Blast Report | Hs.591190 |
| (SEQ ID NO: 1794) | | | factor 12 | | |
| WAN008DUC_at | 1.657 | PHF14 | PHD finger protein 14 | WAN008DUC_at Blast | Hs.159918 |
| (SEQ ID NO: 1795) | | | | Report | |
| AB004109_at | 2.124 | PTDSS2 | Phosphatidylserine | AB004109_at Blast Report | Hs.12851 |
| (SEQ ID NO: 1796) | | | synthase 2 | | |
| WAN0088ZP_at | 1.519 | Pawr | PRKC, apoptosis, WT1, | WAN0088ZP_at Blast | Hs.643130 |
| (SEQ ID NO: 1482) | | | regulator | Report | |
| WAN008E7A_at | 1.93 | PGRMC1 | Progesterone receptor | WAN008E7A_at Blast | Hs.90061 |
| (SEQ ID NO: 1797) | | | membrane component 1 | Report | |
| WAN0088KG_at | 1.642 | PPGB | Protective protein for beta- | WAN0088KG_at Blast | Hs.517076 |
| (SEQ ID NO: 1539) | | | galactosidase | Report | |
| | | | (galactosialidosis) | | |
| WAN0088XS_at | 1.678 | PKN2 | Protein kinase N2 | WAN0088XS_at Blast | Hs.440833 |
| (SEQ ID NO: 1798) | | | | Report | |
| WAN0088YU_at | 1.556 | PPP4C | Protein phosphatase 4 | WAN0088YU_at Blast | Hs.534338 |
| (SEQ ID NO: 1799) | | | (formerly X), catalytic | Report | |
| | | | subunit | | |
| WAN0088X9_at | 1.501 | RAB34 | RAB34, member RAS | WAN0088X9_at Blast | Hs.301853 |
| (SEQ ID NO: 1553) | | | oncogene family | Report | |
| WAN008CLK_at | 1.56 | RAB6A | RAB6A, member RAS | WAN008CLK_at Blast | Hs.12152 |
| (SEQ ID NO: 1552) | | | oncogene family | Report | |
| AB015640_at | 1.554 | RANBP9 | RAN binding protein 9 | AB015640_at Blast Report | Hs.306242 |
| (SEQ ID NO: 1800) | | | | | |
| WAN013HVD_at | 1.569 | RHOA | Ras homolog gene family, | WAN013HVD_at Blast | Hs.247077 |
| (SEQ ID NO: 1801) | | | member A | Report | |
| WAN008DRK_at | 1.738 | RFC4 | Replication factor C | WAN008DRK_at Blast | Hs.591322 |
| (SEQ ID NO: 1802) | | | (activator 1) 4, 37 kDa | Report | |
| WAN008DWV_x_at | 1.717 | Rmnd5a | Required for meiotic nuclear | WAN008DWV_x_at Blast | Hs.75277 |
| (SEQ ID NO: 1803) | | | division 5 homotog A | Report | |
| | | | (S. cerevisiae) | | |
| WAN013I1O_at | 1.543 | RNH1 | Ribonuclease/angiogenin | WAN013I1O_at Blast | Hs.530687 |
| (SEQ ID NO: 1804) | | | inhibitor 1 | Report | |
| WAN008DC4_at | 2.743 | Rpl14 | Ribosomal protein L14 | WAN008DC4_at Blast | Hs.446522 |
| (SEQ ID NO: 1805) | | | | Report | |
| WAN013I36_f_at | 1.889 | RPSA | Ribosomal protein SA | WAN013I36_f_at Blast | Hs.449909 |
| (SEQ ID NO: 1806) | | | | Report | |
| WAN008CKA_at | 1.518 | 1110002B05Rik | RIKEN cDNA 1110002B05 | WAN008CKA_at Blast | #N/A |
| (SEQ ID NO: 1807) | | | gene | Report | |
| WAN008EQG_at | 1.678 | SAPS3 | SAPS domain family, | WAN008EQG_at Blast | Hs.503022 |
| (SEQ ID NO: 1808) | | | member 3 | Report | |
| AF004831_at | 1.999 | SPTLC1 | Serine palmitoyltransferase, | AF004831_at Blast Report | Hs.90458 |
| (SEQ ID NO: 1536) | | | long chain base subunit 1 | | |
| WAN008EE3_at | 1.521 | SARS | Seryl-tRNA synthetase | WAN008EE3_at Blast | Hs.531176 |
| (SEQ ID NO: 1809) | | | | Report | |
| WAN0088TG_at | 1.997 | SRP72 | Signal recognition particle | WAN0088TG_at Blast | Hs.237825 |
| (SEQ ID NO: 1540) | | | 72 kDa | Report | |
| S79122_x_at | 2.103 | SON | SON DNA binding protein | S79122_x_at Blast Report | Hs.517262 |
| (SEQ ID NO: 1810) | | | | | |
| WAN008EFY_at | 1.597 | SPG21 | Spastic paraplegia 21 | WAN008EFY_at Blast | Hs.242458 |
| (SEQ ID NO: 1811) | | | (autosomal recessive, Mast | Report | |
| | | | syndrome) | | |
| WAN008CSS_at | 1.546 | SART1 | Squamous cell carcinoma | WAN008CSS_at Blast | Hs.502883 |
| (SEQ ID NO: 1812) | | | antigen recognised by T | Report | |
| | | | cells | | |
| AF039202_at | 1.603 | STIP1 | Stress-induced- | AF039202_at Blast Report | Hs.337295 |
| (SEQ ID NO: 1813) | | | phosphoprotein 1 | | |
| | | | (Hsp70/Hsp90-organizing | | |
| | | | protein) | | |
| WAN013I25_at | 1.88 | Stx4a | Syntaxin 4A (placental) | WAN013I25_at Blast | #N/A |
| (SEQ ID NO: 1814) | | | | Report | |
| WAN013HX6_at | 1.998 | TAX1BP1 | Tax1 (human T-cell | WAN013HX6_at Blast | Hs.34576 |
| (SEQ ID NO: 1815) | | | leukemia virus type I) | Report | |
| | | | binding protein 1 | | |
| WAN0088ST_at | 1.521 | TBC1D15 | TBC1 domain family, | WAN0088ST_at Blast | Hs.284630 |
| (SEQ ID NO: 1816) | | | member 15 | Report | |
| WAN008D32_at | 1.687 | TSPAN6 | Tetraspanin 6 | WAN008D32_at Blast | Hs.43233 |
| (SEQ ID NO: 1817) | | | | Report | |
| WAN013I98_at | 1.517 | TST | Thiosulfate sulfurtransferase | WAN013I98_at Blast | Hs.474783 |
| (SEQ ID NO: 1818) | | | (rhodanese) | Report | |
| WAN013HUQ_x_at | 1.674 | THOC5 | THO complex 5 | WAN013HUQ_x_at Blast | Hs.75361 |
| (SEQ ID NO: 1819) | | | | Report | |
| L00365_at | 1.673 | TK1 | Thymidine kinase 1, soluble | L00365_at Blast Report | Hs.515122 |
| (SEQ ID NO: 1820) | | | | | |
| WAN008EKQ_at | 1.528 | TIAL1 | TIA1 cytotoxic granule- | WAN008EKQ_at Blast | Hs.501203 |
| (SEQ ID NO: 1821) | | | associated RNA binding | Report | |
| | | | protein-like 1 | | |
| WAN013I4X_at | 1.957 | Timp2 | Tissue inhibitor of | WAN013I4X_at Blast | Hs.633514 |
| (SEQ ID NO: 1742) | | | metalloproteinase 2 | Report | |
| WAN013IA5_at | 1.626 | TOP2B | Topoisomerase (DNA) II | WAN013IA5_at Blast | Hs.475733 |
| (SEQ ID NO: 1822) | | | beta 180 kDa | Report | |
| WAN0088TW_at | 1.537 | TCEB3 | Transcription elongation | WAN0088TW_at Blast | Hs.584806 |
| (SEQ ID NO: 1745) | | | factor B (SIII), polypeptide 3 | Report | |
| | | | (110 kDa, elongin A) | | |
| WAN008DE8_at | 1.553 | TIMM23 | Translocase of inner | WAN008DE8_at Blast | Hs.524308 |
| (SEQ ID NO: 1823) | | | mitochondrial membrane 23 | Report | |
| | | | homolog (yeast) | | |
| WAN008CSZ_at | 1.896 | Tloc1 | Translocation protein 1 | WAN008CSZ_at Blast | Hs.592561 |
| (SEQ ID NO: 1824) | | | | Report | |
| WAN008EFO_at | 1.59 | TM9SF2 | Transmembrane 9 | WAN008EFO_at Blast | Hs.130413 |
| (SEQ ID NO: 1825) | | | superfamily member 2 | Report | |
| WAN008D2L_at | 1.6 | TMEM50A | Transmembrane protein | WAN008D2L_at Blast | Hs.523054 |
| (SEQ ID NO: 1747) | | | 50A | Report | |
| U29167_at | 1.592 | TPM2 | Tropomyosin 2 (beta) | U29167_at Blast Report | Hs.300772 |
| (SEQ ID NO: 1672) | | | | | |
| WAN013IAB_x_at | 1.578 | TP53 | Tumor protein p53 (Li- | WAN013IAB_x_at Blast | Hs.408312 |
| (SEQ ID NO: 1496) | | | Fraumeni syndrome) | Report | |
| WAN008EUV_x_at | 2.038 | Tpt1 | Tumor protein, | WAN008EUV_x_at Blast | Hs.374596 |
| (SEQ ID NO: 1826) | | | translationally-controlled 1 | Report | |
| WAN008E9O_at | 2.17 | TSG101 | Tumor susceptibility gene | WAN008E9O_at Blast | Hs.523512 |
| (SEQ ID NO: 1827) | | | 101 | Report | |
| WAN008DK3_at | 1.574 | YWHAQ | Tyrosine 3- | WAN008DK3_at Blast | Hs.74405 |
| (SEQ ID NO: 1828) | | | monooxygenase/tryptophan | Report | |
| | | | 5-monooxygenase | | |
| | | | activation protein, theta | | |
| | | | polypeptide | | |
| WAN008DK1_at | 1.778 | UQCRC1 | Ubiquinol-cytochrome c | WAN008DK1_at Blast | Hs.119251 |
| (SEQ ID NO: 1829) | | | reductase core protein I | Report | |
| AF004368_at | 1.703 | UGP2 | UDP-glucose | AF004368_at Blast Report | Hs.516217 |
| (SEQ ID NO: 1749) | | | pyrophosphorylase 2 | | |
| WAN008D7G_at | 1.595 | VAMP3 | Vesicle-associated | WAN008D7G_at Blast | Hs.66708 |
| (SEQ ID NO: 1750) | | | membrane protein 3 | Report | |
| | | | (cellubrevin) | | |
| WAN013I7T_at | 1.691 | HRAS | V-Ha-ras Harvey rat | WAN013I7T_at Blast | Hs.37003 |
| (SEQ ID NO: 1830) | | | sarcoma viral oncogene | Report | |
| | | | homolog | | |
| X63416_at | 1.675 | MOS | V-mos Moloney murine | X63416_at Blast Report | Hs.533432 |
| (SEQ ID NO: 1831) | | | sarcoma viral oncogene | | |
| | | | homolog | | |
| S74024_at | 1.772 | XPA | Xeroderma pigmentosum, | S74024_at Blast Report | Hs.591907 |
| (SEQ ID NO: 1832) | | | complementation group A | | |
| WAN008906_at | 1.532 | Zfp259 | Zinc finger protein 259 | WAN008906_at Blast | #N/A |
| (SEQ ID NO: 1833) | | | | Report | |
| WAN008CO7_at | 1.701 | Zfp622 | Zinc finger protein 622 | WAN008CO7_at Blast | #N/A |
| (SEQ ID NO: 1834) | | | | Report | |
| UP (Originally 78) | | | | | |
| L00178_at | 1.828153565 | Hmgcr | 3-hydroxy-3-methylglutaryl- | Hs.643495 | 3E−47 |
| (SEQ ID NO: 1507) | | | Coenzyme A reductase | | |
| L00327_x_at | 2.475247525 | HMGCS1 | 3-hydroxy-3-methylglutaryl- | Hs.397729 | 8E−51 |
| (SEQ ID NO: 1835) | | | Coenzyme A synthase 1 | | |
| | | | (soluble) | | |
| WAN013HXI_at | 1.538461538 | ACAT2 | Acetyl-Coenzyme A | Hs.571037 | 1E−122 |
| (SEQ ID NO: 1836) | | | acetyltransferase 2 | | |
| | | | (acetoacetyl Coenzyme A | | |
| | | | thiolase) | | |
| AF022944_at | 1.834862385 | NA | AF022944 Cricetulus | #N/A | 1E−34 |
| (SEQ ID NO: 1837) | | | griseus DNA damage | | |
| | | | inducible clone similar to | | |
| | | | eukaryotic initiation factor 5 | | |
| | | | (eIF-5) cDNA, partial | | |
| | | | sequence. | | |
| WAN013HTZ_at | 4.464285714 | BCL2L1 | BCL2-like 1 | Hs.516966 | 0 |
| (SEQ ID NO: 1838) | | | | | |
| WAN008CI5_at | 1.550387597 | CDC20 | CDC20 cell division cycle 20 | Hs.524947 | 1E−105 |
| (SEQ ID NO: 1839) | | | homolog (S. cerevisiae) | | |
| M29895_at | 1.508295626 | CHI3L1 | Chitinase 3-like 1 (cartilage | Hs.382202 | 5E−26 |
| (SEQ ID NO: 1840) | | | glycoprotein-39) | | |
| X81405_at | 1.552795031 | EN2 | Engrailed homolog 2 | Hs.134989 | 5E−69 |
| (SEQ ID NO: 1682) | | | | | |
| D11452_at | 1.503759398 | GIP | Gastric inhibitory | Hs.1454 | 3E−35 |
| (SEQ ID NO: 1841) | | | polypeptide | | |
| WAN013I0X_at | 2.008032129 | GSS | Glutathione synthetase | Hs.82327 | 1E−98 |
| (SEQ ID NO: 1581) | | | | | |
| WAN008F1C_x_at | 1.706484642 | GSK3A | Glycogen synthase kinase 3 | Hs.466828 | 2E−16 |
| (SEQ ID NO: 1842) | | | alpha | | |
| WAN0088K7_x_at | 3.558718861 | Hspa5 | Heat shock 70 kD protein 5 | Hs.605502 | 0 |
| (SEQ ID NO: 1506) | | | (glucose-regulated protein) | | |
| WAN013HWO_x_at | 2.141327623 | HSP90B1 | Heat shock protein 90 kDa | Hs.192374 | 2E−48 |
| (SEQ ID NO: 1529) | | | beta (Grp94), member 1 | | |
| L38710_x_at | 1.697792869 | HSD3B1 | Hydroxy-delta-5-steroid | Hs.364941 | 2E−41 |
| (SEQ ID NO: 1843) | | | dehydrogenase, 3 beta- and | | |
| | | | steroid delta-isomerase 1 | | |
| WAN013I9D_at | 1.582278481 | HYOU1 | Hypoxia up-regulated 1 | Hs.277704 | 4E−72 |
| (SEQ ID NO: 1525) | | | | | |
| D89285_at | 1.677852349 | ITIH1 | Inter-alpha (globulin) | Hs.420257 | 2E−80 |
| (SEQ ID NO: 1844) | | | inhibitor H1 | | |
| WAN0088SX_x_at | 1.76366843 | MGEA5 | Meningioma expressed | Hs.500842 | 0.00006 |
| (SEQ ID NO: 1845) | | | antigen 5 (hyaluronidase) | | |
| AB028638_at | 1.680672269 | PDGFB | Platelet-derived growth | Hs.1976 | 4E−68 |
| (SEQ ID NO: 1846) | | | factor beta polypeptide | | |
| | | | (simian sarcoma viral (v-sis) | | |
| | | | oncogene homolog) | | |
| WAN008CWW_at | 1.703577513 | PHTF2 | Putative homeodomain | Hs.203965 | 1E−12 |
| (SEQ ID NO: 1690) | | | transcription factor 2 | | |
| WAN008EIS_at | 1.782531194 | RORB | RAR-related orphan | Hs.494178 | 0 |
| (SEQ ID NO: 1847) | | | receptor B | | |
| WAN008DXF_x_at | 1.623376623 | SENP5 | SUMO1/sentrin specific | Hs.240770 | 1E−33 |
| (SEQ ID NO: 1848) | | | peptidase 5 | | |
| WAN008D13_at | 1.642036125 | TXNRD1 | Thioredoxin reductase 1 | Hs.567352 | 2E−91 |
| (SEQ ID NO: 1849) | | | | | |
| AF022945-rc_f_at | 2.577319588 | Thbd | Thrombomodulin | Hs.2030 | 0 |
| (SEQ ID NO: 1666) | | | | | |
| L22614_at | 1.798561151 | THBS1 | Thrombospondin 1 | Hs.164226 | 3E−62 |
| (SEQ ID NO: 1850) | | | | | |
| L19142_f_at | 2.222222222 | TFRC | Transferrin receptor (p90, | Hs.529618 | 1E−106 |
| (SEQ ID NO: 1851) | | | CD71) | | |
| AY012002_at | 1.703577513 | TYR | Tyrosinase (oculocutaneous | Hs.503555 | 1E−83 |
| (SEQ ID NO: 1852) | | | albinism IA) | | |
| WAN0088P2_at | 1.519756839 | UAP1 | UDP-N-acteylglucosamine | Hs.492859 | 1E−130 |
| (SEQ ID NO: 1853) | | | pyrophosphorylase 1 | | |
| AF271265_at | 1.62601626 | UCP3 | Uncoupling protein 3 | Hs.101337 | 3E−69 |
| (SEQ ID NO: 1854) | | | (mitochondrial, proton | | |
| | | | carrier) | | |
| WAN008ERE_x_at | 1.788908766 | WDR67 | WD repeat domain 67 | Hs.492716 | 9E−36 |
| (SEQ ID NO: 1855) | | | | | |

| | | | Mouse | | | | |
|---|---|---|---|---|---|---|---|
| Qualifier List | % ID | % QC | Unigene ID | eValue | % ID | % QC | |
| DOWN (Originally | | | | | | | |
| 173) | | | | | | | |
| WAN008DUG_at | 0.00000002 | 90.78947 | 13.74322 | Mm.286458 | 3E−43 | 91.55844 | 27.8481 |
| (SEQ ID NO: 1752) | | | | | | | |
| WAN008EB0_at | 1E−49 | 88.64865 | 50.40872 | Mm.296191 | 3E−70 | 93.04813 | 50.95368 |
| (SEQ ID NO: 1699) | | | | | | | |
| WAN008CT8_at | 0 | 94.38445 | 80.80279 | Mm.18946 | 0 | 95.48387 | 81.15183 |
| (SEQ ID NO: 1522) | | | | | | | |
| AF120325_x_at | 0 | 87.58085 | 83.83948 | #N/A | 0 | 91.625 | 86.7679 |
| (SEQ ID NO: 1753) | | | | | | | |
| WAN008EKP_at | 0 | 93.25397 | 99.80198 | Mm.238973 | 0 | 93.2 | 99.0099 |
| (SEQ ID NO: 1754) | | | | | | | |
| WAN008F20_x_at | 1E−26 | 85.31073 | 81.56682 | Mm.389983 | 5E−21 | 89.77273 | 40.553 |
| (SEQ ID NO: 1755) | | | | | | | |
| WAN008DPO_at | 3E−32 | 84.4 | 50.2008 | Mm.71103 | 7E−41 | 87.08134 | 41.96787 |
| (SEQ ID NO: 1756) | | | | | | | |
| WAN013I3P_at | 1E−147 | 86.70213 | 99.29577 | #N/A | 1E−172 | 88.6121 | 98.94366 |
| (SEQ ID NO: 1653) | | | | | | | |
| WAN008DSX_at | 7E−51 | 94.07895 | 38.48101 | Mm.285993 | 6E−84 | 90.80882 | 68.86076 |
| (SEQ ID NO: 1757) | | | | | | | |
| WAN013HVK_at | 1E−164 | 88.38028 | 97.93103 | Mm.247837 | 0 | 94.3761 | 98.10345 |
| (SEQ ID NO: 1758) | | | | | | | |
| WAN008ECX_at | 9E−85 | 86.36364 | 65.0647 | Mm.30246 | 1E−155 | 94.85714 | 64.69501 |
| (SEQ ID NO: 1759) | | | | | | | |
| WAN008D27_at | 0 | 94.06308 | 99.26335 | Mm.298875 | 0 | 94.83395 | 99.81584 |
| (SEQ ID NO: 1760) | | | | | | | |
| WAN008DS9_at | 1E−113 | 89.01869 | 90.87049 | Mm.276826 | 1E−132 | 92.98246 | 84.71338 |
| (SEQ ID NO: 1613) | | | | | | | |
| AF022941_x_at | 9E−27 | 93.65079 | 69.61326 | Mm.17898 | 1E−52 | 96.26866 | 74.03315 |
| (SEQ ID NO: 1761) | | | | | | | |
| WAN008EEB_at | 2E−95 | 90.34483 | 55.98456 | Mm.276859 | 1E−128 | 94.61279 | 57.33591 |
| (SEQ ID NO: 1762) | | | | | | | |
| WAN0088J2_at | 1E−145 | 87.9017 | 98.8785 | Mm.218848 | 1E−156 | 88.18011 | 99.62617 |
| (SEQ ID NO: 1763) | | | | | | | |
| WAN0088PY_at | 0 | 0 | 0 | Mm.220038 | 0.0000002 | 92.85714 | 9.230769 |
| (SEQ ID NO: 1480) | | | | | | | |
| WAN013I1H_at | 1E−167 | 86.99473 | 99.64974 | Mm.261027 | 0 | 88.61646 | 100 |
| (SEQ ID NO: 1764) | | | | | | | |
| WAN008DVF_at | 1E−143 | 89.33333 | 100 | Mm.258140 | 0 | 95.11111 | 100 |
| (SEQ ID NO: 1765) | | | | | | | |
| WAN013I3F_at | 1E−111 | 84.33515 | 98.21109 | Mm.29553 | 0 | 90.87657 | 100 |
| (SEQ ID NO: 1718) | | | | | | | |
| WAN013HZ5_at | 0 | 93.29004 | 83.69565 | Mm.377134 | 0 | 96.31236 | 83.51449 |
| (SEQ ID NO: 1766) | | | | | | | |
| WAN008E8R_at | 1E−122 | 93.13725 | 71.16279 | Mm.27695 | 1E−140 | 95.46926 | 71.86047 |
| (SEQ ID NO: 1767) | | | | | | | |
| WAN013HZP_at | 1E−178 | 97.74648 | 72.44898 | Mm.185453 | 0 | 99.71831 | 72.44898 |
| (SEQ ID NO: 1644) | | | | | | | |
| WAN013IA4_at | 1E−174 | 92.89827 | 98.11676 | Mm.329353 | 0 | 95.2919 | 100 |
| (SEQ ID NO: 1768) | | | | | | | |
| WAN008E82_at | 8E−57 | 87.68657 | 64.57831 | Mm.31941 | 1E−162 | 93.9759 | 100 |
| (SEQ ID NO: 1769) | | | | | | | |
| D43757_at | 6E−15 | 85.04673 | 19.31408 | Mm.88793 | 3E−40 | 82.95455 | 47.65343 |
| (SEQ ID NO: 1770) | | | | | | | |
| WAN008E72_x_at | 6E−25 | 86.13861 | 100 | Mm.153226 | 9E−72 | 94.05941 | 100 |
| (SEQ ID NO: 1547) | | | | | | | |
| WAN008EXR_at | 9E−81 | 86.35015 | 100 | #N/A | 1E−103 | 89.22156 | 99.10979 |
| (SEQ ID NO: 1771) | | | | | | | |
| WAN008EQH_at | 5E−15 | 94.54545 | 12.22222 | Mm.10600 | 1E−57 | 90.26549 | 50.22222 |
| (SEQ ID NO: 1772) | | | | | | | |
| WAN008BR0_at | 0.000001 | 83.95062 | 15.0838 | Mm.230169 | 6E−26 | 88.80597 | 24.95345 |
| (SEQ ID NO: 1773) | | | | | | | |
| WAN008CFZ_at | 6E−18 | 90 | 14.76015 | Mm.358714 | 6E−29 | 91.17647 | 18.81919 |
| (SEQ ID NO: 1774) | | | | | | | |
| WAN013I1P_at | 0 | 97.22222 | 90.94737 | Mm.155896 | 0 | 96.52778 | 90.94737 |
| (SEQ ID NO: 1492) | | | | | | | |
| Y00365_at | 1E−104 | 93.97993 | 23.39593 | Mm.207047 | 1E−145 | 88.84058 | 53.99061 |
| (SEQ ID NO: 1674) | | | | | | | |
| WAN008EKL_at | 1E−120 | 89.60784 | 98.4556 | Mm.390461 | 0 | 91.68279 | 99.80695 |
| (SEQ ID NO: 1775) | | | | | | | |
| WAN013I8N_at | 0 | 90.28974 | 95.36968 | Mm.6065 | 0 | 93.18358 | 96.41524 |
| (SEQ ID NO: 1776) | | | | | | | |
| WAN0088O9_at | 1E−13 | 90.32258 | 16.48936 | Mm.263396 | 4E−55 | 90.49774 | 39.1844 |
| (SEQ ID NO: 1777) | | | | | | | |
| AF180918_at | 6E−21 | 88.57143 | 19.77401 | Mm.10281 | 5E−48 | 86.2069 | 49.15254 |
| (SEQ ID NO: 1778) | | | | | | | |
| WAN008E26_x_at | 7E−11 | 90.90909 | 57.89474 | #N/A | 6E−13 | 87.77778 | 78.94737 |
| (SEQ ID NO: 1779) | | | | | | | |
| WAN008BNG_at | 4E−90 | 93.77778 | 45.91837 | Mm.31247 | 1E−85 | 92.88889 | 45.91837 |
| (SEQ ID NO: 1780) | | | | | | | |
| WAN008EKF_at | 1E−151 | 89.65517 | 98.7234 | Mm.181009 | 0 | 94.20601 | 99.14894 |
| (SEQ ID NO: 1781) | | | | | | | |
| L18986_at | 4E−58 | 85.66176 | 20.76336 | Mm.16716 | 1E−157 | 86.94915 | 45.03817 |
| (SEQ ID NO: 1501) | | | | | | | |
| WAN008F1L_at | 1E−127 | 90.23355 | 84.86486 | Mm.2154 | 1E−131 | 88.44765 | 99.81982 |
| (SEQ ID NO: 1782) | | | | | | | |
| WAN008ESO_at | 0 | 92.12598 | 99.60784 | Mm.41288 | 0 | 94.88189 | 99.60784 |
| (SEQ ID NO: 1783) | | | | | | | |
| J00061_at | 3E−46 | 87.70053 | 66.31206 | Mm.192991 | 2E−64 | 91.89189 | 65.60284 |
| (SEQ ID NO: 1784) | | | | | | | |
| X79864_at | 4E−44 | 85.26786 | 38.75433 | Mm.133851 | 1E−107 | 85.79336 | 93.77163 |
| (SEQ ID NO: 1785) | | | | | | | |
| WAN008E43_at | 6E−86 | 87.12575 | 59.53654 | Mm.26614 | 1E−130 | 89.42308 | 74.1533 |
| (SEQ ID NO: 1786) | | | | | | | |
| WAN008DKS_at | 1E−116 | 92.16867 | 68.73706 | Mm.2720 | 0 | 93.39019 | 97.10145 |
| (SEQ ID NO: 1787) | | | | | | | |
| WAN008CQE_at | 5E−40 | 87.28324 | 33.7232 | Mm.1103 | 1E−128 | 87.47475 | 96.49123 |
| (SEQ ID NO: 1788) | | | | | | | |
| WAN008EE0_x_at | 0.00002 | 88.23529 | 13.31593 | Mm.290791 | 4E−26 | 84.47205 | 42.03655 |
| (SEQ ID NO: 1789) | | | | | | | |
| WAN013I17_at | 1E−101 | 83.47826 | 83.03249 | Mm.4691 | 9E−99 | 91.66667 | 45.48736 |
| (SEQ ID NO: 1790) | | | | | | | |
| WAN008BNY_at | 1E−129 | 92.30769 | 84.0796 | Mm.4467 | 1E−146 | 94.3787 | 84.0796 |
| (SEQ ID NO: 1791) | | | | | | | |
| WAN0088KK_x_at | 1E−30 | 95.58824 | 38.96848 | Mm.1025 | 4E−75 | 91.12903 | 71.06017 |
| (SEQ ID NO: 1792) | | | | | | | |
| WAN013I62_at | 1E−178 | 85.99222 | 56.64952 | Mm.34102 | 0 | 91.76788 | 54.44526 |
| (SEQ ID NO: 1732) | | | | | | | |
| WAN013I2X_at | 1E−129 | 88.28633 | 98.08511 | Mm.11815 | 0 | 93.76344 | 98.93617 |
| (SEQ ID NO: 1793) | | | | | | | |
| AB041733_at | 1E−39 | 92.56198 | 9.173616 | Mm.102205 | 4E−75 | 86.98413 | 23.88173 |
| (SEQ ID NO: 1794) | | | | | | | |
| WAN008DUC_at | 8E−86 | 94.63415 | 99.51456 | Mm.212411 | 4E−74 | 92.19512 | 99.51456 |
| (SEQ ID NO: 1795) | | | | | | | |
| AB004109_at | 0 | 91.24767 | 40.40632 | Mm.293591 | 0 | 89.98288 | 87.88563 |
| (SEQ ID NO: 1796) | | | | | | | |
| WAN0088ZP_at | 4E−10 | 91.52542 | 11.11111 | Mm.391419 | 8E−53 | 91.62562 | 38.22976 |
| (SEQ ID NO: 1482) | | | | | | | |
| WAN008E7A_at | 1E−164 | 91.43426 | 98.04688 | Mm.9052 | 1E−170 | 91.63347 | 98.04688 |
| (SEQ ID NO: 1797) | | | | | | | |
| WAN0088KG_at | 1E−115 | 87.5895 | 72.86957 | Mm.359633 | 1E−149 | 90.93079 | 72.86957 |
| (SEQ ID NO: 1539) | | | | | | | |
| WAN0088XS_at | 8E−13 | 85.04673 | 34.96732 | Mm.244236 | 7E−12 | 84.40367 | 35.62092 |
| (SEQ ID NO: 1798) | | | | | | | |
| WAN0088YU_at | 1E−107 | 94.02985 | 89.63211 | Mm.41998 | 1E−107 | 94.02985 | 89.63211 |
| (SEQ ID NO: 1799) | | | | | | | |
| WAN0088X9_at | 1E−108 | 89.17379 | 66.73004 | Mm.275864 | 1E−161 | 92.15686 | 87.26236 |
| (SEQ ID NO: 1553) | | | | | | | |
| WAN008CLK_at | 2E−55 | 88.47737 | 48.21429 | #N/A | 1E−161 | 92.07317 | 97.61905 |
| (SEQ ID NO: 1552) | | | | | | | |
| AB015640_at | 0 | 95 | 100 | Mm.148781 | 0 | 97.95455 | 100 |
| (SEQ ID NO: 1800) | | | | | | | |
| WAN013HVD_at | 0 | 95.8042 | 100 | Mm.757 | 0 | 96.32867 | 100 |
| (SEQ ID NO: 1801) | | | | | | | |
| WAN008DRK_at | 0.000005 | 86.66667 | 11.07011 | Mm.386835 | 6E−29 | 90.37037 | 24.90775 |
| (SEQ ID NO: 1802) | | | | | | | |
| WAN008DWV_x_at | 3E−59 | 96.83544 | 44.2577 | Mm.28474 | 1E−133 | 95.2381 | 100 |
| (SEQ ID NO: 1803) | | | | | | | |
| WAN013I1O_at | 1E−18 | 83.45324 | 27.8 | Mm.279485 | 1E−91 | 88.23529 | 61.2 |
| (SEQ ID NO: 1804) | | | | | | | |
| WAN008DC4_at | 4E−99 | 88.52941 | 68.41046 | Mm.289810 | 1E−154 | 90.58296 | 89.73843 |
| (SEQ ID NO: 1805) | | | | | | | |
| WAN013I36_f_at | 1E−14 | 89.41176 | 85.85859 | Mm.4071 | 3E−20 | 90.81633 | 98.9899 |
| (SEQ ID NO: 1806) | | | | | | | |
| WAN008CKA_at | 1E−112 | 87.2 | 100 | Mm.292775 | 1E−147 | 90 | 100 |
| (SEQ ID NO: 1807) | | | | | | | |
| WAN008EQG_at | 2E−57 | 87.26708 | 87.02703 | Mm.284686 | 1E−145 | 95.40541 | 100 |
| (SEQ ID NO: 1808) | | | | | | | |
| AF004831_at | 1E−18 | 88.88889 | 6.766917 | Mm.240336 | 5E−84 | 89.44099 | 24.21053 |
| (SEQ ID NO: 1536) | | | | | | | |
| WAN008EE3_at | 1E−116 | 90.80119 | 100 | Mm.28688 | 1E−136 | 93.17507 | 100 |
| (SEQ ID NO: 1809) | | | | | | | |
| WAN0088TG_at | 1E−58 | 89.0411 | 50.81206 | Mm.296976 | 1E−119 | 92.40122 | 76.33411 |
| (SEQ ID NO: 1540) | | | | | | | |
| S79122_x_at | 4E−47 | 98.44961 | 75.88235 | Mm.46401 | 2E−32 | 87.64706 | 100 |
| (SEQ ID NO: 1810) | | | | | | | |
| WAN008EFY_at | 1E−128 | 90.45093 | 71.94656 | Mm.272475 | 1E−136 | 91.44385 | 71.37405 |
| (SEQ ID NO: 1811) | | | | | | | |
| WAN008CSS_at | 0 | 90.3169 | 98.2699 | Mm.34562 | 0 | 94.80969 | 100 |
| (SEQ ID NO: 1812) | | | | | | | |
| AF039202_at | 0 | 88.80208 | 61.39089 | Mm.258633 | 0 | 93.61979 | 61.39089 |
| (SEQ ID NO: 1813) | | | | | | | |
| WAN013I25_at | 3E−81 | 86.88525 | 56.27306 | Mm.24867 | 1E−165 | 88.17006 | 99.8155 |
| (SEQ ID NO: 1814) | | | | | | | |
| WAN013HX6_at | 1E−111 | 86.72986 | 41.49459 | Mm.431979 | 1E−124 | 89.42065 | 39.03638 |
| (SEQ ID NO: 1815) | | | | | | | |
| WAN0088ST_at | 5E−18 | 84.31373 | 52.44216 | Mm.22252 | 3E−67 | 90.7563 | 61.18252 |
| (SEQ ID NO: 1816) | | | | | | | |
| WAN008D32_at | 4E−12 | 100 | 9.87013 | Mm.46701 | 1E−57 | 88.70968 | 64.41558 |
| (SEQ ID NO: 1817) | | | | | | | |
| WAN013I98_at | 0 | 84.09332 | 87.23404 | Mm.15312 | 0 | 90.04329 | 85.47641 |
| (SEQ ID NO: 1818) | | | | | | | |
| WAN013HUQ_x_at | 3E−48 | 90.50633 | 96.34146 | Mm.28969 | 7E−66 | 94.47853 | 99.39024 |
| (SEQ ID NO: 1819) | | | | | | | |
| L00365_at | 6E−24 | 90.32258 | 70.45455 | Mm.2661 | 4E−48 | 94.4 | 94.69697 |
| (SEQ ID NO: 1820) | | | | | | | |
| WAN008EKQ_at | 1E−127 | 92.2043 | 100 | Mm.242072 | 1E−164 | 95.16129 | 100 |
| (SEQ ID NO: 1821) | | | | | | | |
| WAN013I4X_at | 0 | 0 | 0 | Mm.206505 | 0 | 94.2623 | 100 |
| (SEQ ID NO: 1742) | | | | | | | |
| WAN013IA5_at | 0 | 94.43414 | 100 | Mm.130362 | 0 | 98.7013 | 100 |
| (SEQ ID NO: 1822) | | | | | | | |
| WAN0088TW_at | 1E−173 | 89.36567 | 98.52941 | Mm.27663 | 0 | 93.09701 | 98.52941 |
| (SEQ ID NO: 1745) | | | | | | | |
| WAN008DE8_at | 1E−106 | 89.75904 | 100 | Mm.303703 | 1E−127 | 92.66055 | 98.49398 |
| (SEQ ID NO: 1823) | | | | | | | |
| WAN008CSZ_at | 4E−22 | 86.41975 | 43.31551 | Mm.26017 | 1E−125 | 93.18182 | 62.7451 |
| (SEQ ID NO: 1824) | | | | | | | |
| WAN008EFO_at | 1E−153 | 90.94203 | 100 | Mm.275191 | 0 | 95.47101 | 100 |
| (SEQ ID NO: 1825) | | | | | | | |
| WAN008D2L_at | 2E−76 | 85.79882 | 80.09479 | Mm.88349 | 1E−63 | 86.92308 | 61.61137 |
| (SEQ ID NO: 1747) | | | | | | | |
| U29167_at | 0 | 92.83111 | 88.78101 | Mm.646 | 0 | 94.74313 | 90.29126 |
| (SEQ ID NO: 1672) | | | | | | | |
| WAN013IAB_x_at | 1E−150 | 82.44767 | 48.85917 | #N/A | 1E−133 | 81.32045 | 48.85917 |
| (SEQ ID NO: 1496) | | | | | | | |
| WAN008EUV_x_at | 7E−19 | 88.76404 | 36.17886 | Mm.297482 | 1E−65 | 88.21138 | 100 |
| (SEQ ID NO: 1826) | | | | | | | |
| WAN008E9O_at | 1E−49 | 86.76414 | 43.074 | Mm.241334 | 1E−97 | 95.55556 | 42.6945 |
| (SEQ ID NO: 1827) | | | | | | | |
| WAN008DK3_at | 1E−98 | 89.41799 | 100 | Mm.289630 | 1E−151 | 94.17989 | 100 |
| (SEQ ID NO: 1828) | | | | | | | |
| WAN008DK1_at | 3E−69 | 85.66879 | 64.87603 | Mm.335460 | 1E−110 | 91.0828 | 64.87603 |
| (SEQ ID NO: 1829) | | | | | | | |
| AF004368_at | 1E−116 | 91.77215 | 53.92491 | Mm.28877 | 1E−147 | 90.86538 | 70.98976 |
| (SEQ ID NO: 1749) | | | | | | | |
| WAN008D7G_at | 0.0000004 | 97.56098 | 7.400722 | Mm.273930 | 9E−19 | 91.86047 | 15.52347 |
| (SEQ ID NO: 1750) | | | | | | | |
| WAN013I7T_at | 2E−36 | 89.92806 | 24.5583 | #N/A | 1E−171 | 92.13251 | 85.33569 |
| (SEQ ID NO: 1830) | | | | | | | |
| X63416_at | 4E−28 | 86.2069 | 27.30697 | Mm.317339 | 1E−157 | 100 | 52.91902 |
| (SEQ ID NO: 1831) | | | | | | | |
| S74024_at | 4E−54 | 87.73585 | 96.36364 | Mm.247036 | 6E−64 | 89.09091 | 100 |
| (SEQ ID NO: 1832) | | | | | | | |
| WAN008906_at | 1E−162 | 90.1354 | 94.86239 | Mm.17519 | 0 | 92.84404 | 100 |
| (SEQ ID NO: 1833) | | | | | | | |
| WAN008CO7_at | 1E−166 | 89.8 | 88.33922 | Mm.29145 | 0 | 91.56194 | 98.40989 |
| (SEQ ID NO: 1834) | | | | | | | |
| UP (Originally 78) | | | | | | | |
| L00178_at | 90.0621118 | 42.81915 | Mm.316652 | 1E−57 | 93.037975 | 42.02128 | |
| (SEQ ID NO: 1507) | | | | | | | |
| L00327_x_at | 95.23809524 | 97.67442 | Mm.61526 | 2E−41 | 93.220339 | 91.47287 | |
| (SEQ ID NO: 1835) | | | | | | | |
| WAN013HXI_at | 83.06010929 | 98.74101 | Mm.229342 | 0 | 88.321168 | 98.56115 | |
| (SEQ ID NO: 1836) | | | | | | | |
| AF022944_at | 93.2038835 | 59.53757 | #N/A | 2E−44 | 97.087379 | 59.53757 | |
| (SEQ ID NO: 1837) | | | | | | | |
| WAN013HTZ_at | 93.20754717 | 99.43715 | Mm.238213 | 0 | 93.962264 | 99.43715 | |
| (SEQ ID NO: 1838) | | | | | | | |
| WAN008CI5_at | 89.21282799 | 68.6 | Mm.289747 | 1E−142 | 93.586006 | 68.6 | |
| (SEQ ID NO: 1839) | | | | | | | |
| M29895_at | 86.82170543 | 54.66102 | Mm.38274 | 9E−17 | 93.442623 | 25.84746 | |
| (SEQ ID NO: 1840) | | | | | | | |
| X81405_at | 92.59259259 | 81.11588 | Mm.4298 | 7E−30 | 92.631579 | 40.77253 | |
| (SEQ ID NO: 1682) | | | | | | | |
| D11452_at | 94.84536082 | 17.41472 | Mm.248452 | 4E−95 | 87.257618 | 64.81149 | |
| (SEQ ID NO: 1841) | | | | | | | |
| WAN013I0X_at | 90.78498294 | 56.13027 | Mm.252316 | 1E−129 | 95.189003 | 55.74713 | |
| (SEQ ID NO: 1581) | | | | | | | |
| WAN008F1C_x_at | 81.92090395 | 58.22368 | Mm.294664 | 6E−34 | 84.11215 | 70.39474 | |
| (SEQ ID NO: 1842) | | | | | | | |
| WAN0088K7_x_at | 0 | 0 | Mm.330160 | 0.000008 | 100 | 6.923077 | |
| (SEQ ID NO: 1506) | | | | | | | |
| WAN013HWO_x_at | 84.68085106 | 94 | Mm.87773 | 2E−66 | 87.804878 | 98.4 | |
| (SEQ ID NO: 1529) | | | | | | | |
| L38710_x_at | 80.44554455 | 29.51059 | Mm.140811 | IE−134 | 90.024331 | 30.02191 | |
| (SEQ ID NO: 1843) | | | | | | | |
| WAN013I9D_at | 85.49848943 | 26.4166 | Mm.116721 | 1E−122 | 92.236025 | 25.69832 | |
| (SEQ ID NO: 1525) | | | | | | | |
| D89285_at | 82.95218295 | 80.97643 | Mm.3227 | 1E−127 | 87.366167 | 78.61953 | |
| (SEQ ID NO: 1844) | | | | | | | |
| WAN0088SX_x_at | 84.93150685 | 67.59259 | Mm.122725 | 9E−15 | 90.410959 | 67.59259 | |
| (SEQ ID NO: 1845) | | | | | | | |
| AB028638_at | 83.68421053 | 84.63252 | Mm.144089 | 1E−169 | 91.759465 | 100 | |
| (SEQ ID NO: 1846) | | | | | | | |
| WAN008CWW_at | 86.20689655 | 21.75 | Mm.86410 | 1E−17 | 86.868687 | 24.75 | |
| (SEQ ID NO: 1690) | | | | | | | |
| WAN008EIS_at | 92.87054409 | 98.15838 | Mm.234641 | 0 | 94.814815 | 99.44751 | |
| (SEQ ID NO: 1847) | | | | | | | |
| WAN008DXF_x_at | 97.5308642 | 96.42857 | Mm.152890 | 9E−36 | 97.619048 | 100 | |
| (SEQ ID NO: 1848) | | | | | | | |
| WAN008D13_at | 88.74598071 | 64.2562 | Mm.210155 | 1E−118 | 90.168539 | 73.55372 | |
| (SEQ ID NO: 1849) | | | | | | | |
| AF022945-rc_f_at | 0 | 0 | Mm.24096 | 1E−13 | 89.552239 | 65.04854 | |
| (SEQ ID NO: 1666) | | | | | | | |
| L22614_at | 88.80407125 | 29.26284 | Mm.4159 | 1E−129 | 91.759465 | 33.43261 | |
| (SEQ ID NO: 1850) | | | | | | | |
| L19142_f_at | 87.12871287 | 70.26087 | Mm.28683 | 0 | 91.474245 | 97.91304 | |
| (SEQ ID NO: 1851) | | | | | | | |
| AY012002_at | 85.34031414 | 92.27053 | Mm.238127 | 1E−153 | 91.545894 | 100 | |
| (SEQ ID NO: 1852) | | | | | | | |
| WAN0088P2_at | 86.73267327 | 91.98543 | Mm.27969 | 1E−177 | 90.018832 | 96.72131 | |
| (SEQ ID NO: 1853) | | | | | | | |
| AF271265_at | 87.22627737 | 47.32297 | Mm.6254 | 1E−11B | 95.131086 | 46.11399 | |
| (SEQ ID NO: 1854) | | | | | | | |
| WAN008ERE_x_at | 86.12716763 | 98.29545 | Mm.390835 | 2E−57 | 91.32948 | 98.29545 | |
| (SEQ ID NO: 1855) | | | | | | | |

TABLE 19
late comparison

| Qualifier List | Fold | | | Human | | | | | | | |
|---|---|---|---|---|---|---|---|---|---|---|---|
| (SEQ ID NO:) | Change | Symbol | Title | Unigene ID | eValue | % ID | % QC | Mouse Unigene ID | eValue | % ID | % QC |
| Down (Originally 26) | | | | | | | | | | | |
| WAN008EB0_at | 1.656 | ACOT7 | Acyl-CoA thioesterase 7 | Hs.126137 | 1E−49 | 88.64865 | 50.40872 | Mm.296191 | 3E−70 | 93.04813 | 50.95368 |
| (SEQ ID NO: 1699) | | | | | | | | | | | |
| BI431005_x_at | 1.513 | CCDC67 | Coiled-coil domain containing 67 | Hs.436625 | 2E−22 | 82.51366 | 89.26829 | Mm.32237 | 2E−60 | 90.20619 | 94.63415 |
| (SEQ ID NO: 1856) | | | | | | | | | | | |
| WAN0088O9_at | 1.661 | Itgb1 | Integrin beta 1 (fibronectin | Hs.295626 | 1E−13 | 90.32258 | 16.48936 | Mm.263396 | 4E−55 | 90.49774 | 39.1844 |
| (SEQ ID NO: 1777) | | | receptor beta) | | | | | | | | |
| M99691_at | 1.713 | NA | M99691 Hamster retroviral | #N/A | 0 | 0 | 0 | #N/A | 0 | 0 | 0 |
| (SEQ ID NO: 1857) | | | sequence mRNA. | | | | | | | | |
| J00061_at | 1.682 | MT1 | metallothionein I | #N/A | 3E−46 | 87.70053 | 66.31206 | Mm.192991 | 2E−64 | 91.89189 | 65.60284 |
| (SEQ ID NO: 1784) | | | | | | | | | | | |
| AY029611_f_at | 1.569 | OPN1SW | Opsin 1 (cone pigments), short- | Hs.592258 | 0.00000005 | 83.72093 | 91.48936 | Mm.56987 | 5E−25 | 90.42553 | 100 |
| (SEQ ID NO: 1858) | | | wave-sensitive (color blindness, | | | | | | | | |
| | | | tritan) | | | | | | | | |
| U21937_at | 1.723 | Kcnj6 | Potassium inwardly-rectifying | Hs.50927 | 0.000001 | 84.93151 | 13.27273 | Mm.328720 | 2E−19 | 86.36364 | 20 |
| (SEQ ID NO: 1859) | | | channel, subfamily J, member 6 | | | | | | | | |
| WAN0088KG_at | 2.203 | PPGB | Protective protein for beta- | Hs.517076 | 1E−115 | 87.5895 | 72.86957 | Mm.359633 | 1E−149 | 90.93079 | 72.86957 |
| (SEQ ID NO: 1539) | | | galactosidase (galactosialidosis) | | | | | | | | |
| WAN008DC4_at | 2.003 | Rpl14 | Ribosomal protein L14 | Hs.446522 | 4E−99 | 88.52941 | 68.41046 | Mm.289810 | 1E−154 | 90.58296 | 89.73843 |
| (SEQ ID NO: 1805) | | | | | | | | | | | |
| WAN013I36_f_at | 1.619 | RPSA | Ribosomal protein SA | Hs.449909 | 1E−14 | 89.41176 | 85.85859 | Mm.4071 | 3E−20 | 90.81633 | 98.9899 |
| (SEQ ID NO: 1806) | | | | | | | | | | | |
| M87540_at | 1.589 | Scn1a | Sodium channel, voltage-gated, | Hs.22654 | 0 | 94.89051 | 100 | Mm.365737 | 2E−86 | 85.67708 | 93.43066 |
| (SEQ ID ND: 1860) | | | type I, alpha | | | | | | | | |
| U66490_at | 1.574 | STAR | Steroidogenic acute regulator | Hs.521535 | 6E−88 | 87.27811 | 26.02002 | Mm.293314 | 1E−120 | 90.9621 | 26.40493 |
| (SEQ ID NO: 1861) | | | | | | | | | | | |
| WAN013I4X_at | 1.55 | Timp2 | Tissue inhibitor of | Hs.633514 | 0 | 0 | 0 | Mm.206505 | 0 | 94.2623 | 100 |
| (SEQ ID NO: 1742) | | | metalloproteinase 2 | | | | | | | | |
| WAN008DE8_at | 1.552 | TIMM23 | Translocase of inner | Hs.524308 | 1E−106 | 89.75904 | 100 | Mm.303703 | 1E−127 | 92.66055 | 98.49398 |
| (SEQ ID NO: 1823) | | | mitochondrial membrane 23 | | | | | | | | |
| | | | homolog (yeast) | | | | | | | | |
| WAN013I7T_at | 1.524 | HRAS | V-Ha-ras Harvey rat sarcoma | Hs.37003 | 2E−36 | 89.92806 | 24.5583 | #N/A | 1E−171 | 92.13251 | 85.33569 |
| (SEQ ID NO: 1830) | | | viral oncogene homolog | | | | | | | | |
| UP (Originally 170) | | | | | | | | | | | |
| (SEQ ID NO:) | | | | | | | | | | | |
| L00169_at | 2.832861 | Hmgcr | 3-hydroxy-3-methylglutaryl- | Hs.643495 | 2E−19 | 89.53488 | 33.99209 | Mm.316652 | 7E−27 | 94.87179 | 30.83004 |
| (SEQ ID NO: 1508) | | | Coenzyme A reductase | | | | | | | | |
| L00332_at | 2.849003 | HMGCS1 | 3-hydroxy-3-methylglutaryl- | Hs.397729 | 3E−41 | 90.57971 | 95.83333 | Mm.61526 | 1E−42 | 92.1875 | 88.88889 |
| (SEQ ID NO: 1862) | | | Coenzyme A synthase 1 | | | | | | | | |
| | | | (soluble) | | | | | | | | |
| WAN008BQY_at | 1.960784 | PFKFB4 | 6-phosphofructo-2- | Hs.476217 | 8E−87 | 89.32384 | 98.59649 | Mm.132391 | 1E−113 | 92.98246 | 100 |
| (SEQ ID NO: 1863) | | | kinase/fructose-2,6- | | | | | | | | |
| | | | biphosphatase 4 | | | | | | | | |
| WAN013HXI_at | 1.724138 | ACAT2 | Acetyl-Coenzyme A | Hs.571037 | 1E−122 | 83.06011 | 98.74101 | Mm.229342 | 0 | 88.32117 | 98.56115 |
| (SEQ ID NO: 1836) | | | acetyltransferase 2 (acetoacetyl | | | | | | | | |
| | | | Coenzyme A thiolase) | | | | | | | | |
| AF022944_at | 1.642036 | NA | AF022944 Cricetulus griseus | #N/A | 1E−34 | 93.20388 | 59.53757 | #N/A | 2E−44 | 97.08738 | 59.53757 |
| (SEQ ID NO: 1837) | | | DNA damage inducible clone | | | | | | | | |
| | | | similar to eukaryotic initiation | | | | | | | | |
| | | | factor 5 (elF-5) cDNA, partial | | | | | | | | |
| | | | sequence. | | | | | | | | |
| WAN008DJ2_at | 2.061856 | Anapc1 | Anaphase promoting complex | Hs.436527 | 1E−152 | 89.30818 | 96.95122 | Mm.277408 | 0 | 94.96855 | 96.95122 |
| (SEQ ID NO: 1864) | | | subunit 1 | | | | | | | | |
| WAN008DNR_at | 1.694915 | ANKRD32 | Ankyrin repeat domain 32 | Hs.556673 | 1E−67 | 87.64479 | 64.75 | Mm.209730 | 1E−117 | 89.43089 | 92.25 |
| (SEQ ID NO: 1865) | | | | | | | | | | | |
| WAN008EKW_at | 1.776199 | RERE | Arginine-glutamic acid dipeptide | Hs.463041 | 1E−147 | 90.25845 | 97.85992 | Mm.291274 | 0 | 93.77432 | 100 |
| (SEQ ID NO: 1866) | | | (RE) repeats | | | | | | | | |
| WAN008BSS_at | 1.636661 | ATAD2 | ATPase family, AAA domain | Hs.370834 | 7E−51 | 86.74699 | 61.63366 | Mm.221758 | 8E−71 | 91.34615 | 51.48515 |
| (SEQ ID NO: 1867) | | | containing 2 | | | | | | | | |
| WAN013I05_at | 1.577287 | ABCB6 | ATP-binding cassette, sub-family | Hs.107911 | 1E−154 | 87.15084 | 100 | Mm.28663 | 0 | 91.80633 | 100 |
| (SEQ ID NO: 1646) | | | B (MDR/TAP), member 6 | | | | | | | | |
| WAN008DGC_at | 1.623377 | BAIAP2L1 | BAI1-associated protein 2-like 1 | Hs.584939 | 2E−34 | 83.69099 | 100 | Mm.18814 | 3E−75 | 90.98712 | 100 |
| (SEQ ID NO: 1868) | | | | | | | | | | | |
| WAN013HTZ_at | 6.17284 | BCL2L1 | BCL2-like 1 | Hs.516966 | 0 | 93.20755 | 99.43715 | Mm.238213 | 0 | 93.96226 | 99.43715 |
| (SEQ ID NO: 1838) | | | | | | | | | | | |
| WAN008CYH-at | 1.639344 | CALD1 | Caldesmon 1 | Hs.490203 | 1E−152 | 89.07216 | 85.53792 | Mm.308134 | 0 | 92.76896 | 100 |
| (SEQ ID NO: 1869) | | | | | | | | | | | |
| WAN008BSH_at | 1.529052 | CAT | Catalase | Hs.502302 | 6E−16 | 89.28571 | 38.70968 | Mm.4215 | 3E−41 | 89.24731 | 85.71429 |
| (SEQ ID NO: 1558) | | | | | | | | | | | |
| WAN008CI5_at | 1.858736 | CDC20 | CDC20 cell division cycle 20 | Hs.524947 | 1E−105 | 89.21283 | 68.6 | Mm.289747 | 1E−142 | 93.58601 | 68.6 |
| (SEQ ID NO: 1839) | | | homolog (S. cerevisiae) | | | | | | | | |
| WAN013I2T_at | 1.531394 | CBX5 | Chromobox homolog 5 (HP1 | Hs.632724 | 1E−142 | 91.86352 | 72.02268 | Mm.262059 | 1E−168 | 94.75066 | 72.02268 |
| (SEQ ID NO: 1652) | | | alpha homolog, Drosophila) | | | | | | | | |
| WAN008D17_at | 1.531394 | COPA | Coatomer protein complex, | Hs.162121 | 0 | 92.98597 | 96.14644 | Mm.30041 | 0 | 94.32485 | 98.45857 |
| (SEQ ID NO: 1870) | | | subunit alpha | | | | | | | | |
| WAN013HWY_at | 3.968254 | CCDC80 | Coiled-coil domain containing 80 | Hs.477128 | 6E−92 | 86.55914 | 76.07362 | Mm.181074 | 1E−171 | 90.57377 | 99.7955 |
| (SEQ ID NO: 1710) | | | | | | | | | | | |
| WAN008CI3_at | 1.55521 | CCNJ | Cyclin J | Hs.596479 | 1E−145 | 93.71429 | 77.60532 | Mm.309 | 1E−105 | 88.85714 | 77.60532 |
| (SEQ ID NO: 1680) | | | | | | | | | | | |
| U66494_at | 1.552795 | CYP17A1 | Cytochrome P450, family 17, | Hs.438016 | 4E−47 | 81.94842 | 62.21034 | Mm.1262 | 1E−70 | 82.58929 | 79.8574 |
| (SEQ ID NO: 1871) | | | subfamily A, polypeptide 1 | | | | | | | | |
| WAN00893H_at | 1.594896 | DDX23 | DEAD (Asp-Glu-Ala-Asp) box | Hs.130098 | 1E−163 | 90.41667 | 97.36308 | Mm.45725 | 1E−173 | 91.09731 | 97.9716 |
| (SEQ ID NO: 1872) | | | polypeptide 23 | | | | | | | | |
| WAN013HUL_at | 1.605136 | DDX3X | DEAD (Asp-Glu-Ala-Asp) box | Hs.380774 | 1E−78 | 93.75 | 42.27642 | Mm.289662 | 2E−88 | 91.76955 | 49.39024 |
| (SEQ ID NO: 1873) | | | polypeptide 3, X-linked | | | | | | | | |
| WAN0088WF_at | 1.814882 | DHX36 | DEAH (Asp-Glu-Ala-His) box | Hs.446270 | 2E−97 | 92.07547 | 55.67227 | Mm.224233 | 1E−113 | 94.40299 | 56.30252 |
| (SEQ ID NO: 1874) | | | polypeptide 36 | | | | | | | | |
| WAN0088OR_at | 1.74216 | EIF5B | Eukaryotic translation initiation | Hs.158688 | 2E−61 | 91.17647 | 41.04628 | Mm.260943 | 2E−71 | 89.30041 | 48.89336 |
| (SEQ ID NO: 1875) | | | factor 5B | | | | | | | | |
| WAN008DNP_at | 1.776199 | XPO1 | Exportin 1 (CRM1 homolog, | Hs.370770 | 0 | 91.08527 | 98.47328 | Mm.217547 | 0 | 94.84733 | 100 |
| (SEQ ID NO: 1876) | | | yeast) | | | | | | | | |
| WAN008D0K_at | 1.538462 | AA408296 | Expressed sequence AA408296 | #N/A | 1E−110 | 86.77494 | 96.85393 | Mm.173758 | 1E−143 | 89.86486 | 99.77528 |
| (SEQ ID NO: 1877) | | | | | | | | | | | |
| WAN008CPJ_at | 1.538462 | Fdft1 | Farnesyl diphosphate farnesyl | Hs.593928 | 1E−123 | 85.09874 | 99.64222 | Mm.425927 | 0 | 0 | 0 |
| (SEQ ID NO: 1878) | | | transferase 1 | | | | | | | | |
| WAN013I1W_at | 1.52207 | FDPS | Farnesyl diphosphate synthase | Hs.335918 | 1E−146 | 86.11632 | 100 | Mm.39472 | 0 | 90.78947 | 99.81238 |
| (SEQ ID NO: 1879) | | | (farnesyl pyrophosphate | | | | | | | | |
| | | | synthetase, | | | | | | | | |
| | | | dimethylallyltranstransferase, | | | | | | | | |
| | | | geranyltranstransferase) | | | | | | | | |
| WAN008CQY_at | 1.589825 | FBXL11 | F-box and leucine-rich repeat | Hs.124147 | 0 | 93.30544 | 88.3549 | Mm.31941 | 0 | 97.57914 | 99.26063 |
| (SEQ ID NO: 1880) | | | protein 11 | | | | | | | | |
| WAN008CQH_at | 1.923077 | FTSJ3 | FtsJ homolog 3 (E. coli) | Hs.463785 | 1E−82 | 88.44884 | 84.63687 | Mm.29795 | 1E−110 | 91.69329 | 87.43017 |
| (SEQ ID NO: 1881) | | | | | | | | | | | |
| WAN0088Y2_at | 1.529052 | Ggnbp2 | Gametogenetin binding protein 2 | #N/A | 0 | 90.99265 | 100 | Mm.356653 | 0 | 93.56618 | 100 |
| (SEQ ID NO: 1882) | | | | | | | | | | | |
| WAN01310X_at | 1.831502 | GSS | Glutathione synthetase | Hs.82327 | 1E−98 | 90.78498 | 56.13027 | Mm.252316 | 1E−129 | 95.189 | 55.74713 |
| (SEQ ID NO: 1581) | | | | | | | | | | | |
| WAN008DXA_at | 1.669449 | GPD2 | Glycerol-3-phosphate | Hs.512382 | 1E−111 | 86.72769 | 98.6456 | Mm.3711 | 1E−159 | 91.15646 | 99.54853 |
| (SEQ ID NO: 1883) | | | dehydrogenase 2 (mitochondrial) | | | | | | | | |
| AF307847_at | 1.776199 | GAB1 | GRB2-associated binding | Hs.80720 | 7E−98 | 85.99034 | 76.52495 | Mm.277409 | 1E−136 | 89.57346 | 78.0037 |
| (SEQ ID NO: 1884) | | | protein 1 | | | | | | | | |
| WAN008EAH_at | 1.508296 | GTPBP4 | GTP binding protein 4 | Hs.215766 | 1E−126 | 88.42795 | 88.07692 | Mm.41800 | 0 | 95.41485 | 88.07692 |
| (SEQ ID NO: 1885) | | | | | | | | | | | |
| WAN0088K7_x_at | 2.463054 | Hspa5 | Heat shock 70 kD protein 5 | Hs.605502 | 0 | 0 | 0 | Mm.330160 | 0.000008 | 100 | 6.923077 |
| (SEQ ID NO: 1506) | | | (glucose-regulated protein) | | | | | | | | |
| WAN013HWO_x_at | 2.10084 | HSP90B1 | Heat shock protein 90 kDa beta | Hs.192374 | 2E−48 | 84.68085 | 94 | Mm.87773 | 2E−66 | 87.80488 | 98.4 |
| (SEQ ID NO: 1529) | | | (Grp94), member 1 | | | | | | | | |
| WAN008DQ1_at | 1.6 | Herpud2 | HERPUD family member 2 | Hs.599851 | 3E−31 | 95.34884 | 26.95925 | Mm.142843 | 1E−106 | 90.28213 | 100 |
| (SEQ ID NO: 1886) | | | | | | | | | | | |
| WAN008EVU_x_at | 1.610306 | HNRPK | Heterogeneous nuclear | Hs.522257 | 1E−129 | 92.9878 | 59.63636 | Mm.142872 | 1E−170 | 90.5838 | 96.54545 |
| (SEQ ID NO: 1887) | | | ribonucleoprotein K | | | | | | | | |
| L38709_at | 1.706485 | HSD3B | Hydroxy-delta-5-steroid | #N/A | 2E−41 | 82.53425 | 29.46519 | #N/A | 1E−151 | 85.82803 | 63.37033 |
| (SEQ ID NO: 1888) | | | dehydrogenase, 3 beta- and | | | | | | | | |
| | | | steroid delta-isomerase | | | | | | | | |
| WAN013I9D_at | 1.712329 | HYOU1 | Hypoxia up-regulated 1 | Hs.277704 | 4E−72 | 85.49849 | 26.4166 | Mm.116721 | 1E−122 | 92.23602 | 25.69832 |
| (SEQ ID NO: 1525) | | | | | | | | | | | |
| WAN008CF7_at | 1.760563 | IVNS1ABP | Influenza virus NS1A binding | Hs.497183 | 0 | 93.27434 | 100 | Mm.33764 | 0 | 97.34513 | 100 |
| (SEQ ID NO: 1889) | | | protein | | | | | | | | |
| D89285_at | 1.712329 | ITIH1 | Inter-alpha (globulin) inhibitor H1 | Hs.420257 | 2E−80 | 82.95218 | 80.97643 | Mm.3227 | 1E−127 | 87.36617 | 78.61953 |
| (SEQ ID NO: 1844) | | | | | | | | | | | |
| WAN008EX2_x_at | 1.650165 | Ifrd1 | Interferon-related developmental | Hs.7879 | 7E−39 | 90.29851 | 100 | Mm.168 | 5E−63 | 97.76119 | 100 |
| (SEQ ID NO: 1575) | | | regulator 1 | | | | | | | | |
| AF046210_at | 1.706485 | IL10 | Interleukin 10 | Hs.193717 | 3E−65 | 84.14986 | 92.53333 | Mm.874 | 1E−134 | 91.37466 | 98.93333 |
| (SEQ ID NO: 1890) | | | | | | | | | | | |
| X56067_at | 1.724138 | IAPP | Islet amyloid polypeptide | Hs.46835 | 3E−17 | 83.45324 | 20.65379 | Mm.415 | 7E−91 | 88.03681 | 48.43982 |
| (SEQ ID NO: 1891) | | | | | | | | | | | |
| WAN008D55-rc_at | 1.77305 | Lamb1_predicted | Laminin, beta 1 (predicted) | #N/A | 1E−155 | 87.82288 | 97.48201 | #N/A | 1E−161 | 91.66667 | 77.69784 |
| (SEQ ID NO: 1603) | | | | | | | | | | | |
| WAN00895S_at | 1.52439 | LRRC59 | Leucine rich repeat containing | Hs.370927 | 0 | 89.7482 | 100 | Mm.172720 | 0 | 92.6259 | 100 |
| (SEQ ID NO: 1892) | | | 59 | | | | | | | | |
| AF306662_at | 2.008032 | MMP14 | Matrix metallopeptidase 14 | Hs.2399 | 0 | 89.72603 | 99.82906 | Mm.280175 | 0 | 90.08547 | 100 |
| (SEQ ID NO: 1729) | | | (membrane-inserted) | | | | | | | | |
| WAN013I96_at | 1.533742 | MDM2 | Mdm2, transformed 3T3 cell | Hs.567303 | 5E−83 | 86.03896 | 51.41903 | Mm.22670 | 2E−89 | 87.10692 | 53.08848 |
| (SEQ ID NO: 1893) | | | double minute 2, p53 binding | | | | | | | | |
| | | | protein (mouse) | | | | | | | | |
| WAN008EQD_at | 1.718213 | Mx2 | Myxovirus (influenza virus) | Hs.926 | 2E−29 | 80.31746 | 75 | Mm.14157 | 2E−99 | 85.95238 | 100 |
| (SEQ ID NO: 1894) | | | resistance 2 | | | | | | | | |
| WAN0088IR_at | 1.615509 | NQO1 | NAD(P)H dehydrogenase, | Hs.406515 | 1E−42 | 87.57062 | 62.76596 | Mm.252 | 4E−87 | 90.73359 | 91.84397 |
| (SEQ ID NO: 1895) | | | quinone 1 | | | | | | | | |
| WAN008DWL_at | 1.5625 | NEK2 | NIMA (never in mitosis gene a)- | Hs.153704 | 1E−71 | 87.38739 | 58.42105 | Mm.33773 | 1E−152 | 89.13934 | 85.61404 |
| (SEQ ID NO: 1896) | | | related kinase 2 | | | | | | | | |
| WAN013I9Q_f_at | 1.66113 | NKX6-1 | NK6 transcription factor related, | Hs.546270 | 0.00002 | 89.3617 | 10 | Mm.193072 | 5E−26 | 95.89041 | 15.53191 |
| (SEQ ID NO: 1897) | | | locus 1 (Drosophila) | | | | | | | | |
| WAN008ECD_at | 1.697793 | NARG1 | NMDA receptor regulated 1 | Hs.555985 | 0 | 94.43299 | 99.58932 | Mm.275281 | 0 | 96.70103 | 99.58932 |
| (SEQ ID NO: 1898) | | | | | | | | | | | |
| WAN008BT6_at | 1.54321 | NVL | Nuclear VCP-like | Hs.497867 | 1E−86 | 86.31285 | 70.47244 | Mm.263464 | 1E−156 | 91.42212 | 87.20472 |
| (SEQ ID NO: 1899) | | | | | | | | | | | |
| WAN008E89_at | 1.612903 | Nup160 | Nucleoporin 160 | Hs.645358 | 0 | 92.35182 | 98.12383 | Mm.24532 | 0 | 94.3609 | 99.81238 |
| (SEQ ID NO: 1900) | | | | | | | | | | | |
| WAN008EXG_at | 1.547988 | NUP98 | Nucleoporin 98 kDa | Hs.524750 | 1E−108 | 88.66499 | 95.43269 | Mm.215288 | 1E−145 | 91.10577 | 100 |
| (SEQ ID NO: 1901) | | | | | | | | | | | |
| WAN008EVI-rc_at | 2.325581 | Pparbp | Peroxisome proliferator activated | Hs.643754 | 5E−35 | 90.83333 | 51.06383 | Mm.12926 | 2E−76 | 90.6383 | 100 |
| (SEQ ID NO: 1902) | | | receptor binding protein | | | | | | | | |
| WAN008DHY_at | 1.515152 | PIK4CA | Phosphatidylinositol 4-kinase, | Hs.529438 | 1E−172 | 88.44133 | 99.82517 | Mm.5718 | 0 | 92.43243 | 97.02797 |
| (SEQ ID NO: 1903) | | | catalytic, alpha polypeptide | | | | | | | | |
| WAN008CYA_at | 1.577287 | PIR | Pirin (iron-binding nuclear | Hs.495728 | 1E−111 | 86.87783 | 76.60312 | Mm.293463 | 1E−110 | 89.9705 | 58.75217 |
| (SEQ ID NO: 1904) | | | protein) | | | | | | | | |
| WAN013I81_at | 1.644737 | POLD1 | Polymerase (DNA directed), | Hs.279413 | 0 | 86.30952 | 98.31748 | Mm.16549 | 0 | 91.73372 | 100 |
| (SEQ ID NO: 1905) | | | delta 1, catalytic subunit 125 kDa | | | | | | | | |
| WAN008BQZ_at | 1.757469 | KCTD7 | Potassium channel | Hs.546627 | 2E−58 | 91.01124 | 36.47541 | Mm.55812 | 1E−116 | 97.05882 | 48.77049 |
| (SEQ ID NO: 1906) | | | tetramerisation domain | | | | | | | | |
| | | | containing 7 | | | | | | | | |
| WAN008CQA_at | 1.642036 | PKN2 | Protein kinase N2 | Hs.440833 | 0 | 91.79389 | 100 | Mm.244236 | 0 | 93.70229 | 100 |
| (SEQ ID NO: 1907) | | | | | | | | | | | |
| WAN013HXP_at | 1.515152 | PPP1R12A | Protein phosphatase 1, | Hs.49582 | 0 | 93.40463 | 100 | Mm.207499 | 3E−18 | 89.61039 | 13.72549 |
| (SEQ ID NO: 1908) | | | regulatory (inhibitor) subunit 12A | | | | | | | | |
| WAN008E7E_at | 2.03666 | PTAR1 | Protein prenyltransferase alpha | Hs.494100 | 1E−125 | 87.72321 | 92.37113 | Mm.32215 | 1E−169 | 91.32321 | 95.05155 |
| (SEQ ID NO: 1909) | | | subunit repeat containing 1 | | | | | | | | |
| WAN008EXW_at | 1.550388 | PPFIA1 | Protein tyrosine phosphatase, | Hs.530749 | 4E−61 | 85.56338 | 89.87342 | Mm.272809 | 1E−133 | 93.98734 | 100 |
| (SEQ ID NO: 1910) | | | receptor type, f polypeptide | | | | | | | | |
| | | | (PTPRF), interacting protein | | | | | | | | |
| | | | (liprin), alpha 1 | | | | | | | | |
| WAN013I0K_at | 1.610306 | PRPF8 | PRP8 pre-mRNA processing | Hs.181368 | 0 | 88.03419 | 98.81757 | Mm.3757 | 0 | 91.83673 | 99.32432 |
| (SEQ ID NO: 1911) | | | factor 8 homolog (S. cerevisiae) | | | | | | | | |
| WAN008E6L_at | 1.533742 | NA | RC WAN008E6L 11230B-F04 | #N/A | 0 | 0 | 0 | #N/A | 2E−13 | 88.15789 | 17.92453 |
| (SEQ ID NO: 1912) | | | | | | | | | | | |
| WAN008BR5_at | 1.851652 | RCBTB2 | Regulator of chromosome | Hs.25447 | 1E−68 | 89.42731 | 43.90716 | Mm.280068 | 1E−166 | 90.17682 | 98.45261 |
| (SEQ ID NO: 1913) | | | condensation (RCC1) and BTB | | | | | | | | |
| | | | (POZ) domain containing protein 2 | | | | | | | | |
| WAN008DAW_x_at | 1.512859 | RPN1 | Ribophorin I | Hs.518244 | 1E−13 | 91.66667 | 32.43243 | Mm.188544 | 8E−26 | 91.30435 | 49.72973 |
| (SEQ ID NO: 1914) | | | | | | | | | | | |
| WAN013HVB_at | 1.569859 | RNF10 | Ring finger protein 10 | Hs.442798 | 1E−180 | 92.22462 | 80.66202 | Mm.30051 | 1E−158 | 89.56159 | 83.44948 |
| (SEQ ID NO: 1915) | | | | | | | | | | | |
| WAN0088WO_at | 1.976285 | RBM5 | RNA binding motif protein 5 | Hs.439480 | 1E−123 | 93.44262 | 62.37219 | Mm.259197 | 1E−134 | 90.17857 | 91.61554 |
| (SEQ ID NO: 1916) | | | | | | | | | | | |
| WAN008DX4_at | 1.506024 | AHCYL1 | S-adenosylhomocysteine | Hs.485365 | 2E−38 | 93.85965 | 34.65046 | Mm.220328 | 8E−49 | 97.3913 | 34.95441 |
| (SEQ ID NO: 1917) | | | hydrolase-like 1 | | | | | | | | |
| WAN008CR3_at | 1.851852 | SCYL2 | SCY1-like 2 (S. cerevisiae) | Hs.506481 | 1E−120 | 90.2507 | 99.44598 | Mm.27651 | 1E−116 | 90.05682 | 97.50693 |
| (SEQ ID NO: 1918) | | | | | | | | | | | |
| M74776_at | 1.745201 | SERPINA6 | Serpin peptidase inhibitor, clade | Hs.532635 | 0.000006 | 88.46154 | 9.42029 | Mm.290079 | 8E−10 | 82.20339 | 21.37681 |
| (SEQ ID NO: 1919) | | | A (alpha-1 antiproteinase, | | | | | | | | |
| | | | antitrypsin), member 6 | | | | | | | | |
| WAN008EHX_at | 1.594896 | Setd8 | SET domain containing (lysine | Hs.572262 | 1E−141 | 86.65448 | 100 | Mm.137966 | 0 | 89.76234 | 100 |
| (SEQ ID NO: 1920) | | | methyltransferase) 8 | | | | | | | | |
| WAN008DL0_at | 1.66113 | SRP68 | Signal recognition particle 68 kDa | Hs.514495 | 1E−170 | 89.1791 | 96.40288 | Mm.29655 | 0 | 92.85714 | 98.20144 |
| (SEQ ID NO: 1921) | | | | | | | | | | | |
| WAN008EVC_at | 1.694915 | SMYD5 | SMYD family member 5 | Hs.631882 | 1E−85 | 87.19512 | 97.32938 | Mm.219946 | 1E−106 | 89.93902 | 97.32938 |
| (SEQ ID NO: 1922) | | | | | | | | | | | |
| WAN013HU2_at | 1.515152 | SHOC2 | Soc-2 suppressor of clear | Hs.104315 | 1E−161 | 90.12605 | 94.82072 | Mm.228669 | 1E−123 | 86.17234 | 99.40239 |
| (SEQ ID NO: 1923) | | | homolog (C. elegans) | | | | | | | | |
| WAN00BDZS_at | 1.620746 | SLC4A7 | Solute carrier family 4, sodium | Hs.250072 | 0 | 90.19608 | 100 | Mm.258893 | 0 | 95.18717 | 100 |
| (SEQ ID NO: 1924) | | | bicarbonate cotransporter, | | | | | | | | |
| | | | member 7 | | | | | | | | |
| WAN008CMM_at | 1.73913 | SF3B2 | Splicing factor 3b, subunit 2, | Hs.406423 | 1E−142 | 88.4696 | 84.87544 | Mm.196532 | 0 | 91.23435 | 99.46619 |
| (SEQ ID NO: 1925) | | | 145 kDa | | | | | | | | |
| WAN013HVW_at | 1.988072 | Scd1 | Stearoyl-Coenzyme A | #N/A | 0 | 0 | 0 | Mm.193096 | 8E−07 | 88.67925 | 9.330986 |
| (SEQ ID NO: 1926) | | | desaturase 1 | | | | | | | | |
| U22819_s_at | 2.207506 | SREBF2 | Sterol regulatory element | Hs.443258 | 1E−118 | 89.83516 | 99.45355 | Mm.38016 | 1E−133 | 92.39437 | 96.99454 |
| (SEQ ID NO: 1927) | | | binding transcription factor 2 | | | | | | | | |
| WAN008E8B_at | 1.512859 | TAOK2 | TAO kinase 2 | Hs.291623 | 0 | 91.35135 | 100 | Mm.259634 | 0 | 95.13514 | 100 |
| (SEQ ID NO: 1928) | | | | | | | | | | | |
| WAN008DMR_f_at | 1.506024 | Taok3 | TAO kinase 3 | #N/A | 1E−141 | 90.97744 | 100 | Mm.248296 | 1E−180 | 95.20202 | 99.24812 |
| (SEQ ID NO: 1929) | | | | | | | | | | | |
| WAN008DIO_at | 1.824818 | TIPARP | TCDD-inducible poly(ADP- | Hs.12813 | 1E−114 | 88.65979 | 69.90991 | Mm.246398 | 1E−180 | 89.8917 | 99.81982 |
| (SEQ ID NO: 1930) | | | ribose) polymerase | | | | | | | | |
| WAN0013HV0_at | 3.021148 | TXNRD1 | Thioredoxin reductase 1 | Hs.567352 | 5E−94 | 80.21583 | 96.02763 | Mm.210155 | 1E−105 | 84.29561 | 74.78411 |
| (SEQ ID NO: 1931) | | | | | | | | | | | |
| AF022945-rc_f_at | 1.592357 | Thbd | Thrombomodulin | Hs.2030 | 0 | 0 | 0 | Mm.24096 | 1E−13 | 89.55224 | 65.04854 |
| (SEQ ID NO: 1666) | | | | | | | | | | | |
| WAN013I3R_at | 2.881844 | THRAP4 | Thyroid hormone receptor | Hs.462983 | 2E−63 | 89.15094 | 37.65542 | Mm.248493 | 6E−89 | 91.80328 | 43.33925 |
| (SEQ ID NO: 1932) | | | associated protein 4 | | | | | | | | |
| WAN008DOW_at | 2.717391 | TRIP12 | Thyroid hormone receptor | Hs.591633 | 3E−45 | 87.30159 | 91.74757 | Mm.209265 | 1E−74 | 92.23301 | 100 |
| (SEQ ID NO: 1933) | | | interactor 12 | | | | | | | | |
| L19142_f_at | 2.624672 | TFRC | Transferrin receptor (p90, CD71) | Hs.529618 | 1E−128 | 85.6102 | 68.28358 | Mm.28683 | 0 | 90.125 | 99.50249 |
| (SEQ ID NO: 1851) | | | | | | | | | | | |
| WAN008CZR_at | 1.512859 | TMED1 | Transmembrane emp24 protein | Hs.515139 | 1E−139 | 87.57515 | 90.72727 | Mm.196618 | 0 | 94.89194 | 92.54545 |
| (SEQ ID NO: 1746) | | | transport domain containing 1 | | | | | | | | |
| WAN008D6R_at | 1.519757 | TMED4 | Transmembrane emp24 protein | Hs.510745 | 1E−110 | 91.23377 | 72.30047 | Mm.254495 | 1E−145 | 92.41192 | 86.61972 |
| (SEQ ID NO: 1604) | | | transport domain containing 4 | | | | | | | | |
| WAN008EVD_at | 1.526718 | UBE3B | Ubiquitin protein ligase E3B | Hs.374067 | 1E−116 | 89.88764 | 78.76106 | Mm.28792 | 1E−133 | 91.85393 | 78.76106 |
| (SEQ ID NO: 1934) | | | | | | | | | | | |
| WAN008E4F_at | 1.893939 | USP9X | Ubiquitin specific peptidase 9, X- | Hs.77578 | 1E−180 | 91.45129 | 100 | Mm.242646 | 0 | 94.63221 | 100 |
| (SEQ ID NO: 1935) | | | linked | | | | | | | | |
| WAN013HVL_at | 1.579779 | UGDH | UDP-glucose dehydrogenase | Hs.572518 | 1E−165 | 90.06772 | 95.88745 | Mm.344831 | 1E−158 | 89.01099 | 98.48485 |
| (SEQ ID NO: 1639) | | | | | | | | | | | |
| WAN0088P2_at | 1.923077 | UAP1 | UDP-N-acteylglucosamine | Hs.492859 | 1E−130 | 86.73267 | 91.98543 | Mm.27969 | 1E−177 | 90.01883 | 96.72131 |
| (SEQ ID NO: 1853) | | | phyrophosphorylase 1 | | | | | | | | |
| WAN008EH0_at | 2.024291 | YES1 | V-yes-1 Yamaguchi sarcoma | Hs.194148 | 1E−81 | 92.79279 | 100 | Mm.4558 | 1E−86 | 93.69369 | 100 |
| (SEQ ID NO: 1936) | | | viral oncogene homolog 1 | | | | | | | | |
| WAN008EC3_at | 1.519757 | WDFY3 | WD repeat and FYVE domain | Hs.480116 | 4E−31 | 89.34426 | 24.64646 | Mm.332522 | 1E−57 | 87.86408 | 41.61616 |
| (SEQ ID NO: 1937) | | | containing 3 | | | | | | | | |
| WAN008ERE_x_at | 2.336449 | WDR67 | WD repeat domain 67 | Hs.492716 | 9E−36 | 86.12717 | 98.29545 | Mm.390835 | 2E−57 | 91.32948 | 98.29545 |
| (SEQ ID NO: 1855) | | | | | | | | | | | |
| WAN008E97_at | 1.569859 | WDR76 | WD repeat domain 76 | Hs.250154 | 6E−20 | 83.01887 | 52.82392 | #N/A | 1E−37 | 87.5 | 53.15615 |
| (SEQ ID NO: 1938) | | | | | | | | | | | |
| X56207_at | 1.703578 | NA | X56207 Hamster gene for | #N/A | 0 | 0 | 0 | #N/A | 0.00005 | 92.10526 | 6.713781 |
| (SEQ ID NO: 1696) | | | myosin heavy chain, exons 1 & 2 | | | | | | | | |
| X65592_at | 1.54321 | NA | X65592 C. griseus DNA | #N/A | 0 | 0 | 0 | #N/A | 0 | 0 | 0 |
| (SEQ ID NO: 1939) | | | sequence for interstitial telomere | | | | | | | | |
| | | | associated sequence 1 | | | | | | | | |
| WAN008DKJ_x_at | 1.615509 | Zfp297b | Zinc finger protein 297B | #N/A | 1E−27 | 91.57895 | 90.47619 | Mm.44186 | 2E−24 | 89.69072 | 92.38095 |
| (SEQ ID NO: 1697) | | | | | | | | | | | |

### Example 10

### Platform Analysis

Four cell lines were analyzed from the Platform Process category that exhibit a desired metabolic phenotype when cultured in fed batch culture. That is, the cell lines maintain high viability, and consume lactate and ammonia late in fed batch culture. Multiple time points were collected for each cell line grown in fed batch culture. The time points from each cell line were examined by ANOVA analysis to monitor the changes in gene expression over the course of the culture. The gene lists from each cell line were compared, and those that were in common between all 4 cell lines were identified. Exemplary nucleic acid sequences are listed in Table 20.

TABLE 20
PlatForm Analysis

| | | | Human | | | | Mouse | | | |
|---|---|---|---|---|---|---|---|---|---|---|
| Qualifier List | Symbol | Title | Unigene ID | eValue | % ID | % QC | Unigene ID | eValue | % ID | % QC |
| AF022941_x_at | Cirbp | Cold inducible RNA binding protein | Hs.634522 | 9E−27 | 93.65079 | 69.61326 | Mm.17898 | 1E−52 | 96.26866 | 74.03315 |
| (SEQ ID NO: 1761) | | | | | | | | | | |
| AF081141_at | CCL2 | Chemokine (C-C motif) ligand 2 | Hs.303649 | 3E−13 | 90.625 | 13.41719 | Mm.290320 | 5E−41 | 91.04478 | 28.09224 |
| (SEQ ID NO: 1667) | | | | | | | | | | |
| AF254572_at | ORC1L | Origin recognition complex, subunit 1-like (yeast) | Hs.17908 | 0 | 85.62005 | 63.06156 | Mm.294154 | 0 | 89.31624 | 97.33777 |
| (SEQ ID NO: 1940) | | | | | | | | | | |
| L00366_x_at | TK1 | Thymidine kinase 1, soluble | Hs.515122 | 4E−18 | 89.87342 | 84.94624 | Mm.2661 | 1E−16 | 88.75 | 86.02151 |
| (SEQ ID NO: 1941) | | | | | | | | | | |
| M12329_at | NA | M12329 Chinese hamster alpha-tubulin III mRNA, complete cds. | #N/A | 0 | 93.01676 | 54.28355 | #N/A | 0 | 96.47391 | 53.75284 |
| (SEQ ID NO: 1942) | | | | | | | | | | |
| M80243-rc_at | BIRC5 | Baculoviral IAP repeat-containing 5 (survivin) | Hs.514527 | 4E−38 | 92.37288 | 20.34483 | Mm.8552 | 1E−36 | 93.45794 | 18.44828 |
| (SEQ ID NO: 1943) | | | | | | | | | | |
| U11790_at | KIF2C | Kinesin family member 2C | Hs.69360 | 0 | 89.25714 | 66.43888 | Mm.247651 | 0 | 92.51055 | 71.98178 |
| (SEQ ID NO: 1944) | | | | | | | | | | |
| U48852_at | CRELD2 | Cysteine-rich with EGF-like domains 2 | Hs.211282 | 1E−109 | 81.76944 | 55.13673 | Mm.292567 | 0 | 88.79936 | 91.7221 |
| (SEQ ID NO: 1502) | | | | | | | | | | |
| WAN0088J9_x_at | NA | WAN0088J9 10595A-E01 | #N/A | 0 | 0 | 0 | #N/A | 0 | 0 | 0 |
| (SEQ ID NO: 1588) | | | | | | | | | | |
| WAN0088K2_at | DUSP16 | Dual specificity phosphatase 16 | Hs.536535 | 0.00002 | 84.44444 | 15.98579 | Mm.3994 | 4E−21 | 87.5 | 22.73535 |
| (SEQ ID NO: 1945) | | | | | | | | | | |
| WAN0088ON_at | ATAD2 | ATPase family, AAA domain containing 2 | Hs.370834 | 2E−45 | 83.7037 | 59.08096 | Mm.221758 | 9E−71 | 87.31884 | 60.39387 |
| (SEQ ID NO: 1946) | | | | | | | | | | |
| WAN0088Q6_at | NA | WAN0088Q6 10595D-A09 | #N/A | 1E−153 | 93.71585 | 63.43154 | #N/A | 0 | 94.43155 | 74.69671 |
| (SEQ ID NO: 1590) | | | | | | | | | | |
| WAN0088S8_at | SLC29A1 | Solute carrier family 29 (nucleoside transporters), | Hs.25450 | 3E−35 | 81.35593 | 76.12903 | Mm.29744 | 5E−97 | 86.09756 | 88.17204 |
| (SEQ ID NO: 1591) | | member 1 | | | | | | | | |
| WAN0088T7_at | Cyp51 | Cytochrome P450, family 51 | #N/A | 1E−132 | 86.87873 | 98.05068 | Mm.46044 | 1E−152 | 88.51485 | 98.44055 |
| (SEQ ID NO: 1504) | | | | | | | | | | |
| WAN0088U6_at | SPAG5 | Sperm associated antigen 5 | Hs.514033 | 1E−108 | 84.43649 | 98.07018 | Mm.24250 | 1E−153 | 87.12522 | 99.47368 |
| (SEQ ID NO: 1947) | | | | | | | | | | |
| WAN0088X5_at | MAD2L1 | MAD2 mitotic arrest deficient-like 1 (yeast) | Hs.591697 | 1E−111 | 87.4092 | 93.01802 | Mm.290830 | 1E−153 | 90.95128 | 97.07207 |
| (SEQ ID NO: 1948) | | | | | | | | | | |
| WAN008906_at | Zfp259 | Zinc finger protein 259 | #N/A | 1E−162 | 90.1354 | 94.86239 | Mm.17519 | 0 | 92.84404 | 100 |
| (SEQ ID NO: 1833) | | | | | | | | | | |
| WAN00893Z_at | NA | WAN00893Z 10599B-D03 | #N/A | 8E−33 | 85.87571 | 30.62284 | #N/A | 3E−77 | 88.25503 | 51.55709 |
| (SEQ ID NO: 1949) | | | | | | | | | | |
| WAN008BNE_x_at | NA | WAN008BNE 11233D-H09 | #N/A | 0 | 0 | 0 | #N/A | 0 | 0 | 0 |
| (SEQ ID NO: 1950) | | | | | | | | | | |
| WAN008BNO_at | 2810025M15Rik | RIKEN cDNA 2810025M15 gene | #N/A | 9E−08 | 83.90805 | 15.90494 | Mm.286863 | 1E−146 | 88.09524 | 92.13894 |
| (SEQ ID NO: 1951) | | | | | | | | | | |
| WAN008BRX_at | RETSAT | Retinol saturase (all-trans-retinol 13,14-reductase) | Hs.440401 | 5E−74 | 83.52403 | 80.18349 | Mm.305108 | 0 | 91.37615 | 100 |
| (SEQ ID NO: 1952) | | | | | | | | | | |
| WAN008BSS_at | ATAD2 | ATPase family, AAA domain containing 2 | Hs.370834 | 7E−51 | 86.74699 | 61.63366 | Mm.221758 | 8E−71 | 91.34615 | 51.48515 |
| (SEQ ID NO: 1867) | | | | | | | | | | |
| WAN008CI5_at | CDC20 | CDC20 cell division cycle 20 homolog (S. cerevisiae) | Hs.524947 | 1E−105 | 89.21283 | 68.6 | Mm.289747 | 1E−142 | 93.58601 | 68.6 |
| (SEQ ID NO: 1839) | | | | | | | | | | |
| WAN008CLU_at | Emp1 | Epithelial membrane protein 1 | Hs.436298 | 0 | 0 | 0 | Mm.182785 | 3E−28 | 90.16393 | 21.66963 |
| (SEQ ID NO: 1953) | | | | | | | | | | |
| WAN008CRT_at | ALG14 | Asparagine-linked glycosylation 14 homolog | Hs.408927 | 4E−47 | 88.39779 | 32.43728 | Mm.269881 | 5E−51 | 88.77005 | 33.51254 |
| (SEQ ID NO: 1954) | | (yeast) | | | | | | | | |
| WAN008CS2_at | VKORC1L1 | Vitamin K epoxide reductase complex, subunit | Hs.427232 | 1E−168 | 91.89189 | 96.73203 | Mm.288718 | 0 | 97.28507 | 96.2963 |
| (SEQ ID NO: 1694) | | 1-like 1 | | | | | | | | |
| WAN008CSG_at | Mthfd1 | Methylenetetrahydrofolate dehydrogenase | Hs.614936 | 1E−147 | 86.8705 | 100 | Mm.29584 | 0 | 90.57971 | 99.28058 |
| (SEQ ID NO: 1955) | | (NADP+ dependent), methenyltetrahydrofolate | | | | | | | | |
| | | cyclohydrolase, formyltetrahydrofolate synthase | | | | | | | | |
| WAN008CT2_at | NA | WAN008CT2 10602B-C08 | #N/A | 2E−98 | 92.39544 | 47.0483 | #N/A | 1E−112 | 94.05204 | 48.12165 |
| (SEQ ID NO: 1956) | | | | | | | | | | |
| WAN008CTA_at | NOLC1 | Nucleolar and coiled-body phosphoprotein 1 | Hs.523238 | 1E−101 | 89.12387 | 59.63964 | Mm.402190 | 3E−28 | 89.90826 | 19.63964 |
| (SEQ ID NO: 1957) | | | | | | | | | | |
| WAN008CVX_at | CDC20 | CDC20 cell division cycle 20 homolog (S. cerevisiae) | Hs.524947 | 1E−169 | 90.6639 | 85.15901 | Mm.289747 | 0 | 92.30769 | 87.27915 |
| (SEQ ID NO: 1958) | | | | | | | | | | |
| WAN008CX4_at | MCM5 | MCM5 minichromosome maintenance | Hs.517582 | 1E−152 | 87.10247 | 100 | Mm.5048 | 0 | 91.48936 | 99.64664 |
| (SEQ ID NO: 1959) | | deficient 5, cell division cycle 46 (S. cerevisiae) | | | | | | | | |
| WAN008CXZ_at | UMPS | Uridine monophosphate synthetase (orotate | Hs.2057 | 1E−135 | 86.13139 | 99.63636 | Mm.13145 | 0 | 91.43898 | 99.81818 |
| (SEQ ID NO: 1960) | | phosphoribosyl transferase and orotidine-5′- | | | | | | | | |
| | | decarboxylase) | | | | | | | | |
| WAN008CYY_at | BUB1B | BUB1 budding uninhibited by benzimidazoles | Hs.631699 | 4E−34 | 81.32184 | 73.10924 | Mm.29133 | 2E−71 | 84.59384 | 75 |
| (SEQ ID NO: 1961) | | 1 homolog beta (yeast) | | | | | | | | |
| WAN008CZP_at | NA | WAN008CZP 10604A-A08 | #N/A | 3E−29 | 90.65421 | 21.44289 | #N/A | 7E−50 | 82.69231 | 62.52505 |
| (SEQ ID NO: 1962) | | | | | | | | | | |
| WAN008D06_at | MCM4 | MCM4 minichromosome maintenance | Hs.460184 | 1E−159 | 87.97814 | 98.21109 | Mm.1500 | 0 | 92.98561 | 99.46333 |
| (SEQ ID NO: 1963) | | deficient 4 (S. cerevisiae) | | | | | | | | |
| WAN008D31_at | Lss | Lanosterol synthase | Hs.596543 | 1E−80 | 85.30184 | 68.27957 | Mm.55075 | 1E−150 | 91.52542 | 74.01434 |
| (SEQ ID NO: 1964) | | | | | | | | | | |
| WAN008D7X_at | NA | WAN008D7X 11164B-D06 | #N/A | 4E−16 | 88.23529 | 16.73228 | #N/A | 1E−109 | 91.23377 | 60.62992 |
| (SEQ ID NO: 1965) | | | | | | | | | | |
| WAN008DBR_at | LUC7L | LUC7-like (S. cerevisiae) | Hs.16803 | 0 | 93.66197 | 100 | Mm.386921 | 0 | 95.07042 | 100 |
| (SEQ ID NO: 1966) | | | | | | | | | | |
| WAN008DGK_at | CHAF1A | Chromatin assembly factor 1, subunit A (p150) | Hs.79018 | 1E−83 | 91.32231 | 57.89474 | Mm.391010 | 1E−101 | 90.84746 | 70.57416 |
| (SEQ ID NO: 1967) | | | | | | | | | | |
| WAN008DK1_at | UQCRC1 | Ubiquinol-cytochrome c reductase core protein I | Hs.119251 | 3E−69 | 85.66879 | 64.87603 | Mm.335460 | 1E−110 | 91.0828 | 64.87603 |
| (SEQ ID NO: 1829) | | | | | | | | | | |
| WAN008DMP_at | EWSR1 | Ewing sarcoma breakpoint region 1 | Hs.374477 | 1E−157 | 90.52863 | 94.19087 | Mm.142822 | 0 | 92.98246 | 94.60581 |
| (SEQ ID NO: 1968) | | | | | | | | | | |
| WAN008DO3_at | ACIN1 | Apoptotic chromatin condensation inducer 1 | Hs.124490 | 2E−54 | 89.2562 | 62.85714 | Mm.297078 | 2E−59 | 84.94318 | 91.42857 |
| (SEQ ID NO: 1969) | | | | | | | | | | |
| WAN008DRM_at | EPHX1 | Epoxide hydrolase 1, microsomal (xenobiotic) | Hs.89649 | 9E−85 | 87.98701 | 60.39216 | Mm.9075 | 1E−113 | 91.22257 | 62.54902 |
| (SEQ ID NO: 1503) | | | | | | | | | | |
| WAN008DWL_at | NEK2 | NIMA (never in mitosis gene a)-related kinase 2 | Hs.153704 | 1E−71 | 87.38739 | 58.42105 | Mm.33773 | 1E−152 | 89.13934 | 85.61404 |
| (SEQ ID NO: 1896) | | | | | | | | | | |
| WAN008DXL_at | NA | WAN008DXL 11229A-C02 | #N/A | 5E−58 | 89.74359 | 44.72477 | #N/A | 2E−83 | 94.92386 | 45.18349 |
| (SEQ ID NO: 1970) | | | | | | | | | | |
| WAN008DZY_at | MCM7 | MCM7 minichromosome maintenance | Hs.438720 | 3E−99 | 88.37209 | 100 | Mm.241714 | 1E−123 | 91.27907 | 100 |
| (SEQ ID NO: 1971) | | deficient 7 (S. cerevisiae) | | | | | | | | |
| WAN008E3C_at | Ptma | Prothymosin alpha | Hs.459927 | 2E−67 | 93.83886 | 44.98934 | Mm.19187 | 1E−148 | 92.74005 | 91.04478 |
| (SEQ ID NO: 1972) | | | | | | | | | | |
| WAN008E3O_at | LINCR | Likely ortholog of mouse lung-inducible | Hs.149219 | 3E−19 | 84.61538 | 38.0117 | Mm.389110 | 3E−76 | 85.33724 | 99.7076 |
| (SEQ ID NO: 1973) | | Neutralized-related C3HC4 RING domain protein | | | | | | | | |
| WAN008E4X_at | NA | WAN008E4X 11230A-D06 | #N/A | 0 | 0 | 0 | #N/A | 0 | 0 | 0 |
| (SEQ ID NO: 1974) | | | | | | | | | | |
| WAN008E4Z_at | Nup153 | Nucleoporin 153 | Hs.601591 | 1E−169 | 89.96063 | 92.53188 | Mm.255398 | 0 | 93.75 | 99.08925 |
| (SEQ ID NO: 1975) | | | | | | | | | | |
| WAN008E5L_at | SLC1A5 | Solute carrier family 1 (neutral amino acid | Hs.631582 | 8E−42 | 84.16667 | 45.62738 | Mm.1056 | 1E−115 | 87.67123 | 83.26996 |
| (SEQ ID NO: 1619) | | transporter), member 5 | | | | | | | | |
| WAN008E65_at | ERP29 | Endoplasmic reticulum protein 29 | Hs.75841 | 1E−164 | 91.04803 | 79.79094 | Mm.154570 | 1E−171 | 90.98532 | 83.10105 |
| (SEQ ID NO: 1976) | | | | | | | | | | |
| WAN008E6I_at | NA | WAN008E6I 11230B-F07 | #N/A | 1E−43 | 88.95706 | 42.22798 | #N/A | 3E−76 | 85.38682 | 90.41451 |
| (SEQ ID NO: 1977) | | | | | | | | | | |
| WAN008EED_at | Sc5d | Sterol-C5-desaturase (fungal ERG3, delta-5- | #N/A | 2E−42 | 85.44601 | 40.72658 | Mm.32700 | 9E−99 | 87.70492 | 69.98088 |
| (SEQ ID NO: 1521) | | desaturase) homolog (S. cerevisae) | | | | | | | | |
| WAN008EJV_at | Racgap1 | Rac GTPase-activating protein 1 | Hs.645513 | 1E−103 | 86.39618 | 93.31849 | Mm.273804 | 1E−133 | 89.31116 | 93.76392 |
| (SEQ ID NO: 1978) | | | | | | | | | | |
| WAN008EK5- | NA | WAN008EK5 11232A-G08 | #N/A | 2E−35 | 92.66055 | 26.65037 | #N/A | 6E−44 | 94.78261 | 28.11736 |
| rc_f_at | | | | | | | | | | |
| (SEQ ID NO: 1979) | | | | | | | | | | |
| WAN008EML_at | PBK | PDZ binding kinase | Hs.104741 | 5E−52 | 89.50276 | 39.09287 | Mm.24337 | 3E−80 | 89.78102 | 59.17927 |
| (SEQ ID NO: 1980) | | | | | | | | | | |
| WAN008EMN_at | NA | WAN008EMN 11232B-E01 | #N/A | 0 | 0 | 0 | #N/A | 0 | 0 | 0 |
| (SEQ ID NO: 1981) | | | | | | | | | | |
| WAN008EP0_at | NA | WAN008EP0 11232C-B07 | #N/A | 0 | 0 | 0 | #N/A | 0.00003 | 95.12195 | 10.90426 |
| (SEQ ID NO: 1982) | | | | | | | | | | |
| WAN008ET3_at | NA | WAN008ET3 11233A-C09 | #N/A | 3E−47 | 85.65574 | 50.30928 | #N/A | 1E−133 | 89.27739 | 88.45361 |
| (SEQ ID NO: 1983) | | | | | | | | | | |
| WAN008ETA_at | Usp40 | Ubiquitin specific peptidase 40 | Hs.96513 | 0 | 0 | 0 | Mm.80484 | 3E−46 | 84.72222 | 50.08696 |
| (SEQ ID NO: 1984) | | | | | | | | | | |
| WAN008EXF_at | KIF11 | Kinesin family member 11 | Hs.8878 | 4E−28 | 86.86131 | 27.56539 | Mm.42203 | 3E−24 | 91.86047 | 17.30382 |
| (SEQ ID NO: 1985) | | | | | | | | | | |
| WAN008F1A_at | CYC1 | Cytochrome c-1 | Hs.289271 | 1E−124 | 86.82008 | 89.34579 | Mm.29196 | 0 | 92.42424 | 98.69159 |
| (SEQ ID NO: 1986) | | | | | | | | | | |
| WAN013HV4_at | NA | Cluster includes WAN008F09 10599A-D09 | #N/A | 5E−09 | 97.2973 | 7.07457 | #N/A | 5E−20 | 86.92308 | 24.8566 |
| (SEQ ID NO: 1987) | | | | | | | | | | |
| WAN013HVE_at | NARS | Asparaginyl-tRNA synthetase | Hs.465224 | 1E−104 | 85.0211 | 79.5302 | Mm.29192 | 0 | 92.22904 | 82.04698 |
| (SEQ ID NO: 1988) | | | | | | | | | | |
| WAN013HW1_at | Eef1d | Eukaryotic translation elongation factor 1 delta | Hs.333388 | 1E−115 | 84.05797 | 99.45946 | Mm.258927 | 0 | 91.24088 | 98.73874 |
| (SEQ ID NO: 1989) | | (guanine nucleotide exchange protein) | | | | | | | | |
| WAN013HW5_at | RPL10A | Ribosomal protein L10a | Hs.546269 | 1E−164 | 89.09465 | 98.98167 | Mm.336955 | 0 | 91.85336 | 100 |
| (SEQ ID NO: 1990) | | | | | | | | | | |
| WAN013HWL_at | EBP | Emopamil binding protein (sterol isomerase) | Hs.632801 | 6E−21 | 84.17266 | 24.86583 | Mm.27183 | 2E−46 | 91.9708 | 24.50805 |
| (SEQ ID NO: 1991) | | | | | | | | | | |
| WAN013HX8_x_at | EIF4A2 | Eukaryotic translation initiation factor 4A, | Hs.518475 | 2E−75 | 94.08602 | 68.50829 | Mm.260084 | 0 | 92.50936 | 98.34254 |
| (SEQ ID NO: 1490) | | isoform 2 | | | | | | | | |
| WAN013HXG_at | NA | Cluster includes WAN008CY6 10604A-H03 | #N/A | 1E−103 | 88.0814 | 62.54545 | #N/A | 1E−118 | 89.14286 | 63.63636 |
| (SEQ ID NO: 1992) | | | | | | | | | | |
| WAN013HZA_at | CSE1L | CSE1 chromosome segregation 1-like (yeast) | Hs.90073 | 1E−180 | 88.51351 | 100 | Mm.22417 | 0 | 93.07432 | 100 |
| (SEQ ID NO: 1993) | | | | | | | | | | |
| WAN013I03_at | RPL8 | Ribosomal protein L8 | Hs.178551 | 1E−166 | 88.00705 | 97.92746 | Mm.30066 | 0 | 92.91883 | 100 |
| (SEQ ID NO: 1994) | | | | | | | | | | |
| WAN013I06_at | NA | Cluster includes WAN008E0Q 11229C-H06 | #N/A | 1E−111 | 85.15284 | 84.34622 | #N/A | 1E−143 | 87.71552 | 85.4512 |
| (SEQ ID NO: 1995) | | | | | | | | | | |
| WAN013I0L_at | SND1 | Staphylococcal nuclease domain containing 1 | Hs.122523 | 1E−156 | 87.89683 | 99.40828 | #N/A | 0 | 91.51874 | 100 |
| (SEQ ID NO: 1996) | | | | | | | | | | |
| WAN013I2L_at | NA | Cluster includes WAN0088QX 10596B-F05 | #N/A | 0 | 0 | 0 | #N/A | 0 | 0 | 0 |
| (SEQ ID NO: 1651) | | | | | | | | | | |
| WAN013I2T_at | CBX5 | Chromobox homolog 5 (HP1 alpha homolog, | Hs.632724 | 1E−142 | 91.86352 | 72.02268 | Mm.262059 | 1E−168 | 94.75066 | 72.02268 |
| (SEQ ID NO: 1652) | | Drosophila) | | | | | | | | |
| WAN013I3N_at | NA | Cluster includes WAN00893W 10599B-D08 | #N/A | 6E−29 | 87.31343 | 40.36145 | #N/A | 7E−76 | 92.57426 | 60.84337 |
| (SEQ ID NO: 1997) | | | | | | | | | | |
| WAN013I5T_at | CCNB1 | Cyclin B1 | Hs.23960 | 1E−93 | 85.30259 | 28.11994 | Mm.260114 | 1E−110 | 87.17949 | 28.44408 |
| (SEQ ID NO: 1998) | | | | | | | | | | |
| WAN013I6G_at | NA | Cluster includes M12252 Chinese hamster | #N/A | 0 | 0 | 0 | #N/A | 0 | 0 | 0 |
| (SEQ ID NO: 1999) | | alpha-tubulin I mRNA, complete cds. | | | | | | | | |
| WAN013I81_at | POLD1 | Polymerase (DNA directed), delta 1, catalytic | Hs.279413 | 0 | 86.30952 | 98.31748 | Mm.16549 | 0 | 91.73372 | 100 |
| (SEQ ID NO: 1905) | | subunit 125 kDa | | | | | | | | |
| WAN013I8D_at | PARP1 | Poly (ADP-ribose) polymerase family, member 1 | Hs.177766 | 2E−43 | 85.57692 | 35.01684 | Mm.277779 | 1E−102 | 87.78055 | 67.50842 |
| (SEQ ID NO: 2000) | | | | | | | | | | |
| WAN013I8J_at | CCNB2 | Cyclin B2 | Hs.194698 | 1E−173 | 86.9258 | 44.39216 | Mm.22592 | 0 | 90.70946 | 46.43137 |
| (SEQ ID NO: 2001) | | | | | | | | | | |
| WAN013I8N_at | IMPDH2 | IMP (inosine monophosphate) dehydrogenase 2 | Hs.476231 | 0 | 90.28974 | 95.36968 | Mm.6065 | 0 | 93.18358 | 96.41524 |
| (SEQ ID NO: 1776) | | | | | | | | | | |
| WAN013I8R_at | Rps2 | Ribosomal protein S2 | Hs.356366 | 0 | 90.22298 | 99.14966 | Mm.157452 | 0 | 95.05119 | 99.65986 |
| (SEQ ID NO: 2002) | | | | | | | | | | |
| WAN013I9O_at | TUBB6 | Tubulin, beta 6 | Hs.193491 | 0 | 92.2528 | 71.39738 | Mm.181860 | 0 | 91.74573 | 76.71033 |
| (SEQ ID NO: 2003) | | | | | | | | | | |
| WAN013I9R_at | NA | Cluster includes Y08202 C. griseus mRNA for | #N/A | 1E−104 | 84.573 | 63.46154 | #N/A | 1E−142 | 88.98072 | 63.46154 |
| (SEQ ID NO: 2004) | | RAD51 protein | | | | | | | | |
| WAN013IAD_at | TOP2A | Topoisomerase (DNA) II alpha 170 kDa | Hs.156346 | 3E−37 | 80.62678 | 29.52061 | Mm.4237 | 1E−86 | 84.11633 | 37.59462 |
| (SEQ ID NO: 2005) | | | | | | | | | | |
| WAN013IAQ- | CDKN1A | Cyclin-dependent kinase inhibitor 1A (p21, | Hs.370771 | 2E−10 | 100 | 14.1129 | Mm.195663 | 1E−31 | 88.88889 | 50.80645 |
| rc_x_at | | Cip1) | | | | | | | | |
| (SEQ ID NO: 2006) | | | | | | | | | | |
| X83575_at | KIF23 | Kinesin family member 23 | Hs.270845 | 1E−177 | 92.47788 | 37.07957 | Mm.259374 | 0 | 91.99372 | 52.25595 |
| (SEQ ID NO: 2007) | | | | | | | | | | |
| X83576_at | KIFC1 | Kinesin family member C1 | Hs.436912 | 0 | 86.77111 | 86.84807 | Mm.335713 | 0 | 90.52369 | 90.92971 |
| (SEQ ID NO: 2292) | | | | | | | | | | |

### Example 11

### Target Validation: siRNA

The ability of the differentially expressed genes and proteins to affect a cellular phenotype is verified by overexpression of a nucleic acid inhibiting the expression of the relevant gene using methods known in the art. Exemplary methods based on interfering RNA constructs are described below.

Design and Synthesis of siRNA

Typically, targets that are candidates for siRNA mediated gene knockdown are sequenced, and the sequences verified. Full-length cDNA sequence information is preferred (although not required) to facilitate siRNAs design. The target sequence that is a candidate for gene knockdown is compared to gene sequences available on public or proprietary databases (e.g., BLAST search). Sequences within the target gene that overlap with other known sequences (for example, 16-17 contiguous basepairs of homology) are generally not suitable targets for specific siRNA-mediated gene knockdown.

siRNAs may be designed using, for example, online design tools, over secure internet connections, such as the one available on the Ambion® website (http://www.ambion.com/techlib/misc/siRNA_finder.html). Alternatively, custom siRNAs may also be requested from Ambion®, which applies the Cenix algorithm for designing effective siRNAs. The standard format for siRNAs is typically 5 nmol, annealed and with standard purity in plates. Upon receipt of synthesized siRNAs, the siRNAs are prepared according to the instructions provided by the manufacture and stored at the appropriate temperature (−20° C.)

Standard procedures were used for siRNA transfections. Cells to be transfected were typically pre-passaged on the day before transfection to ensure that the cells are in logarithmic growth phase. Typically, an siRNA Fed-Batch assay was used. Exemplary materials, conditions and methods for transfections are as follows.

Transfection (D0)

Per Spin Tube (50 ml)

100 uL R1

2 uL Transit-TKO transfection reagent (Mirus)

10 uL 10 uM siRNA

2 mL 1 e5 cells/mL in AS1 medium

Following Transfection

37° C.: 72 hrs

31° C.: 96 hrs

Feed: AQ3 on day 3 (D3)

Sample taken on day 1 (D1), day 3 (D3), day 7 (D7)

24 Well Suspension Transfections

For each experiment, 100,000 cells (e.g., 3C7 cells) in 1 mL total volume, and 50 nM siRNA were used. To make a mix for 3 reactions, 150 μL R1 and 70 μL Mirus TKO reagent were mixed and incubated for 10 minutes at room temperature. 15 μL of 10 μM siRNA was added and the mix was incubated for 10 minutes at room temperature. 57.3 μL of the mix was transferred into each of 3 wells. 942.7 μL of R5CD1 (containing 100,000 cells) was added and the plate was incubated on rocker at 37° C. for 72 hrs.

Spin Tube siRNA Transfection

For each experiment, 100,000 cells (e.g., 3C7 cells) in 1 mL total volume were used. For each transfection, 100 μL R1 and 2 μL Mirus TKO reagent were mixed and incubated for 10 minutes at room temperature. 10 μL of 10 μM siRNA was added and the mix was incubated for 15 minutes at room temperature, mixed occasionally. 1.9 mL culture was transferred to each spin tube. siRNA mix (112 uL) was added to each spin tube. The culture was initially incubated at 37° C. and then the temperature was shifted to 31° C. on day 3. Spin tube cultures were shaken rapidly (˜250 RPM). Samples were taken on days 1, 3, and 7. Cultures were terminated on day 7.

Growth and productivity controls were included on each plate. An exemplary productivity control is DHFR (selectable marker on bicistronic mRNA). Treatment with DHFR siRNA reproducibly decreases amount of antibody in the CM-FcIGEN (antibody production control). An exemplary growth control is CHO1 (kinesin) (see Matuliene et al. (2002) Mol. Cell. Biol. 13:1832-45) (typically, about 20-30% growth inhibition was observed with CHO1 treatment). Other standard controls such as no siRNA treatment (transfection reagents only) and non-targeting siRNA treatment (non-specific siRNA) were also included. Plates were then subjected to cell counting (for example, in a 96-well cell counting instrument) to assess growth and to, for example, an automated 96-well titer assay, to assess productivity. Genes whose modulation, singly or in combination, are sufficient to modify useful cellular phenotypes were thereby validated and such changes can be engineered, singly or in combination, into a mammalian cell line to modify its properties.

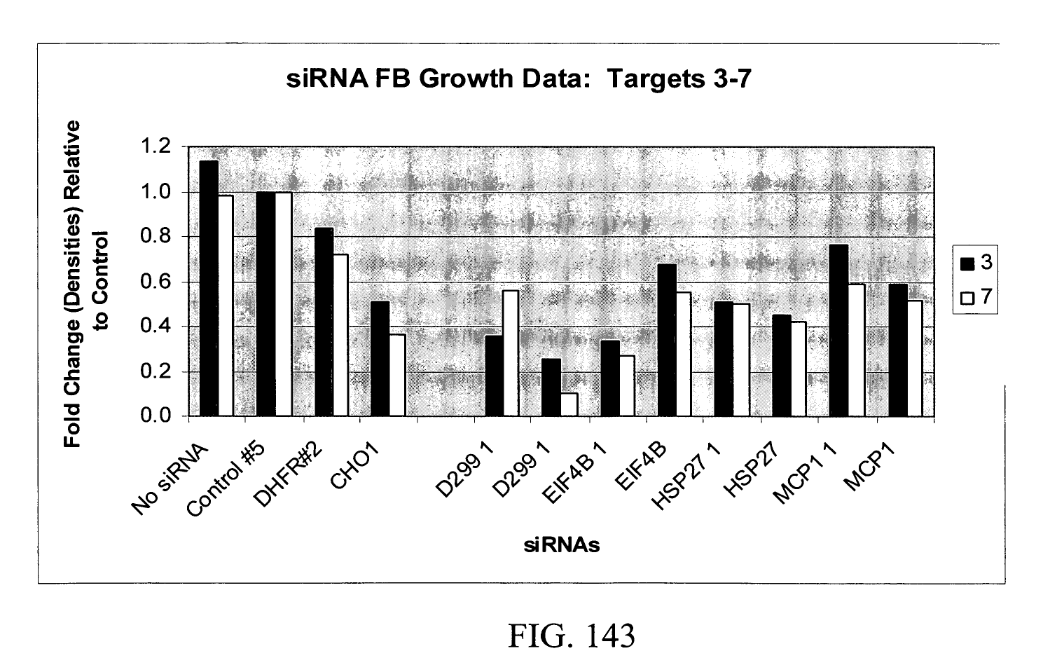

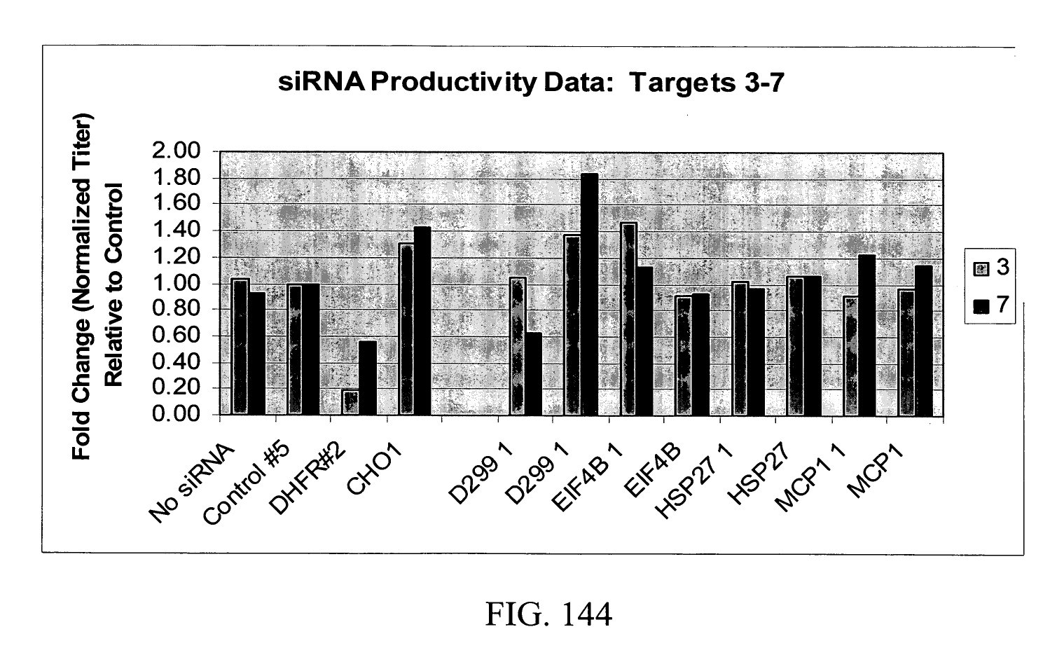

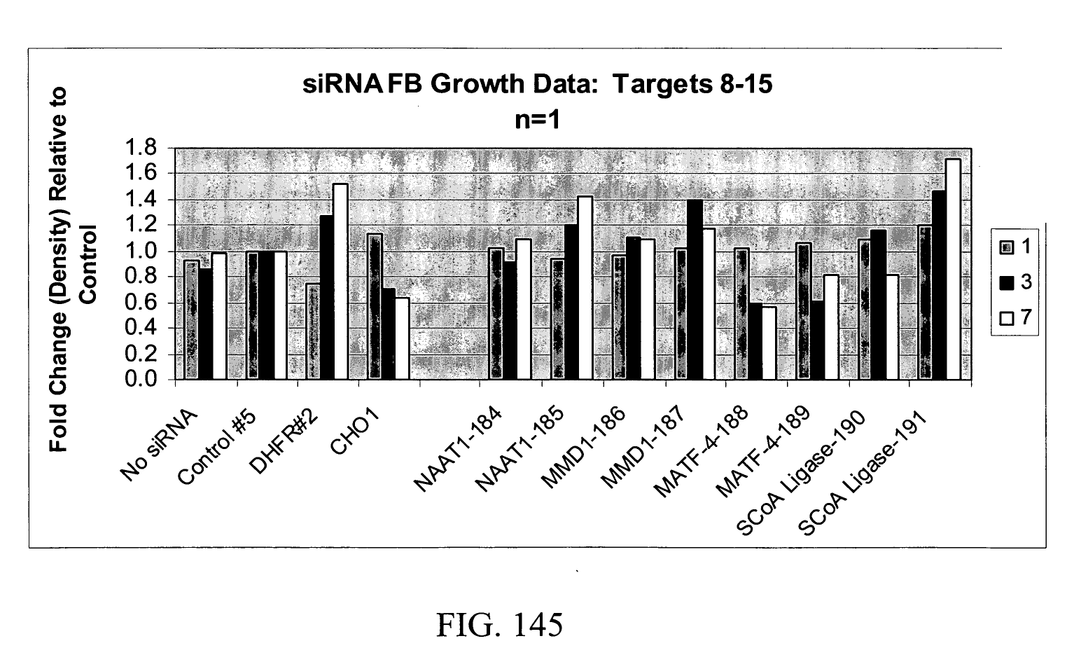

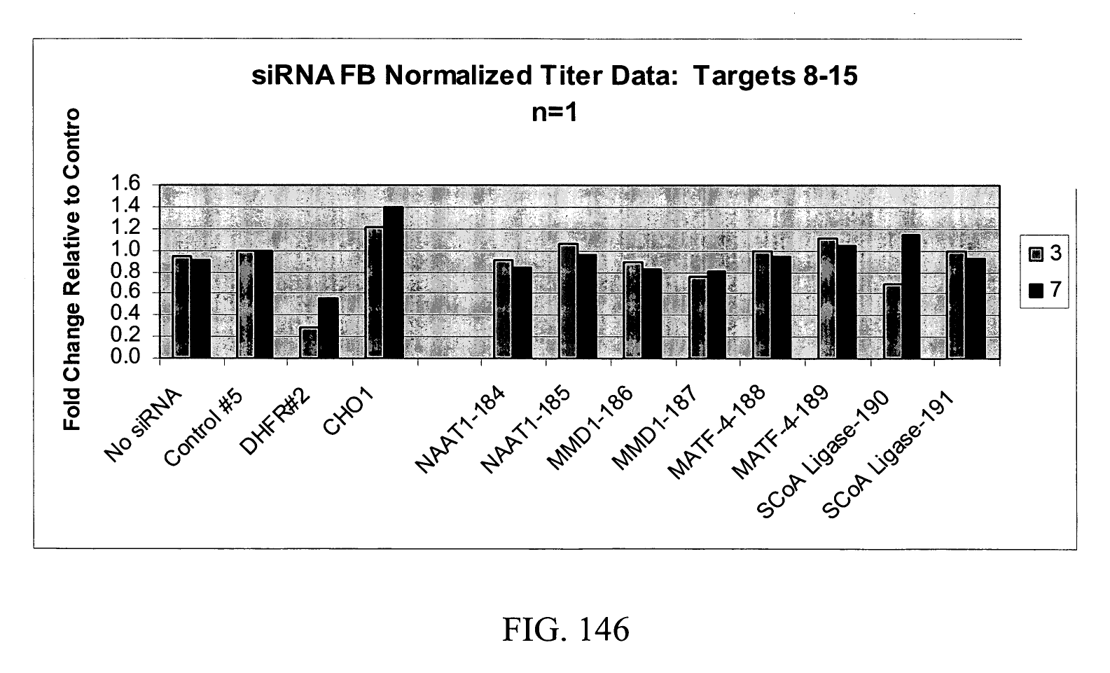

Model cell lines used for the validation purposes and their characteristics are shown in Table 21. FIGS. 143-146 summarize the evaluation of some of the target genes in the spin tube format in the 3C7 cell line. Target genes evaluated include D299 (WAN01318K), identified above as elevated in cells with elevated growth rates; EIF4B, identified above as elevated in cells with elevated growth rates; HSP27 (HSPB1), identified above as elevated in cells with elevated growth rates; MCP1 (CCL2), identified above as depressed in cells with high cell density; NAAT1 (SLC1A4), identified above as depressed in cells with elevated growth rates; MMD1 (malate dehydrogenase), identified above as depressed in cells with high maximum cellular productivities; MATF-4 (ATF-4), identified above as elevated in cells with high cell densities; and SCoA Ligase (SUCLG2), identified above as elevated in cells with high cell densities. As shown in FIG. 143, for genes identified as elevated in cells with elevated growth rates, inhibition of the gene led to an inhibition of growth relative to the control. Cellular productivity was generally not comparably affected, as shown in FIG. 144.

TABLE 21
Cell lines and their characteristics

| Clone | Characteristics |
|---|---|
| 1.14 | High growth rate; Low Qp |
| 1.18 | Average |
| 2.8 | High Qp; High cell density |
| 2B6 | Low cell density; Low Qp |
| DA-4 | Low cell density; Low GR |
| DD-11 | Average |
| DE-6 | Average |
| 3B12 | High Qp; High cell density |
| 5C10 | Average |
| 5B5 | Low Qp |
| 3C7 | Average |

### Example 12

### Target Validation: Overexpression

The ability of the differentially expressed genes and proteins to affect a cellular phenotype is verified by overexpression of a nucleic acid encoding the expression of the relevant gene using methods known in the art. Exemplary methods are described below.

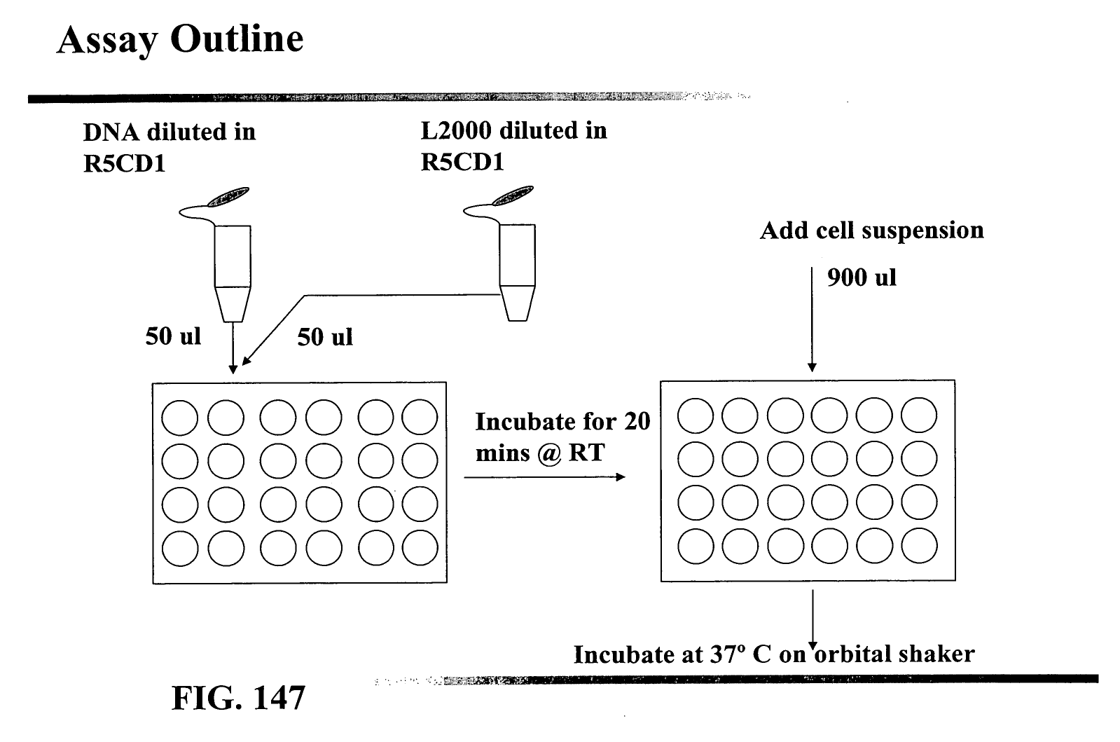

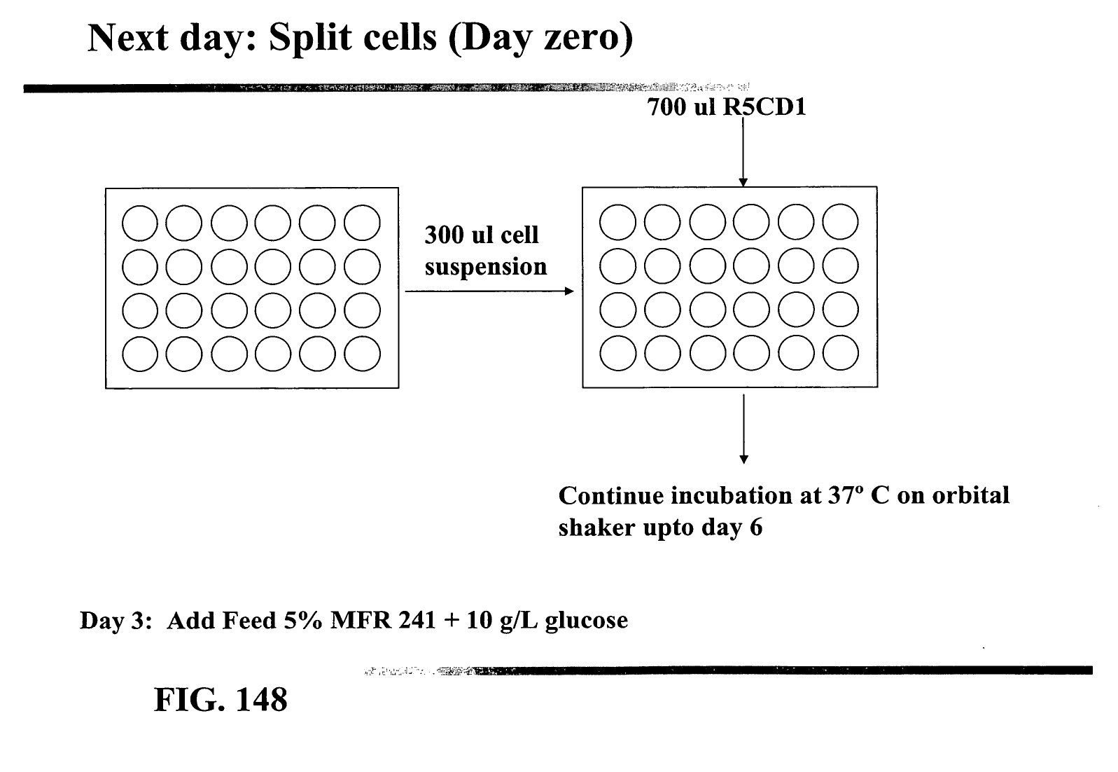

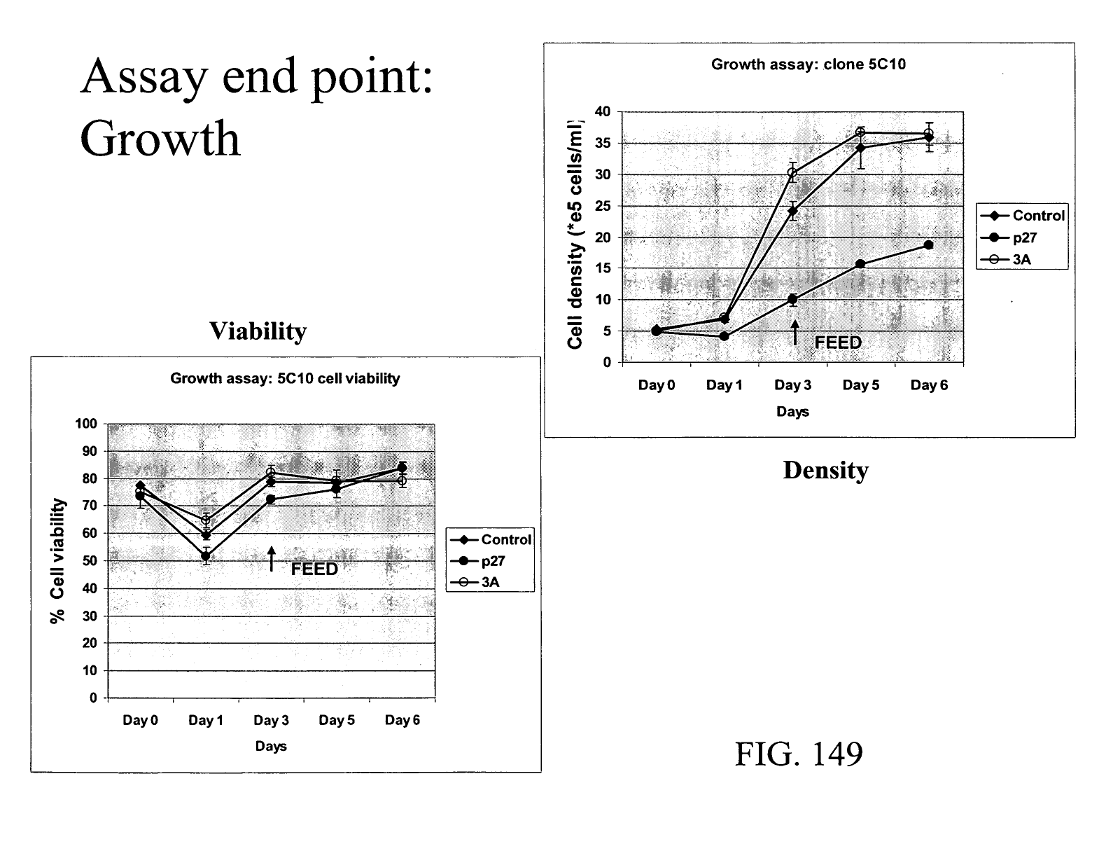

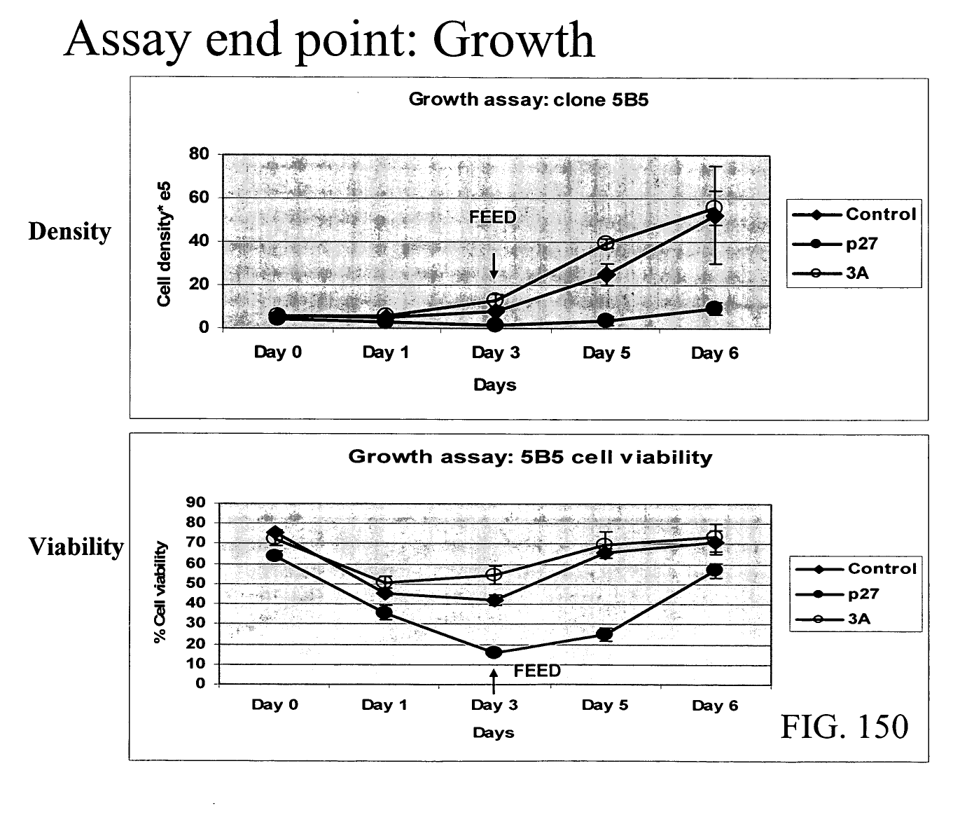

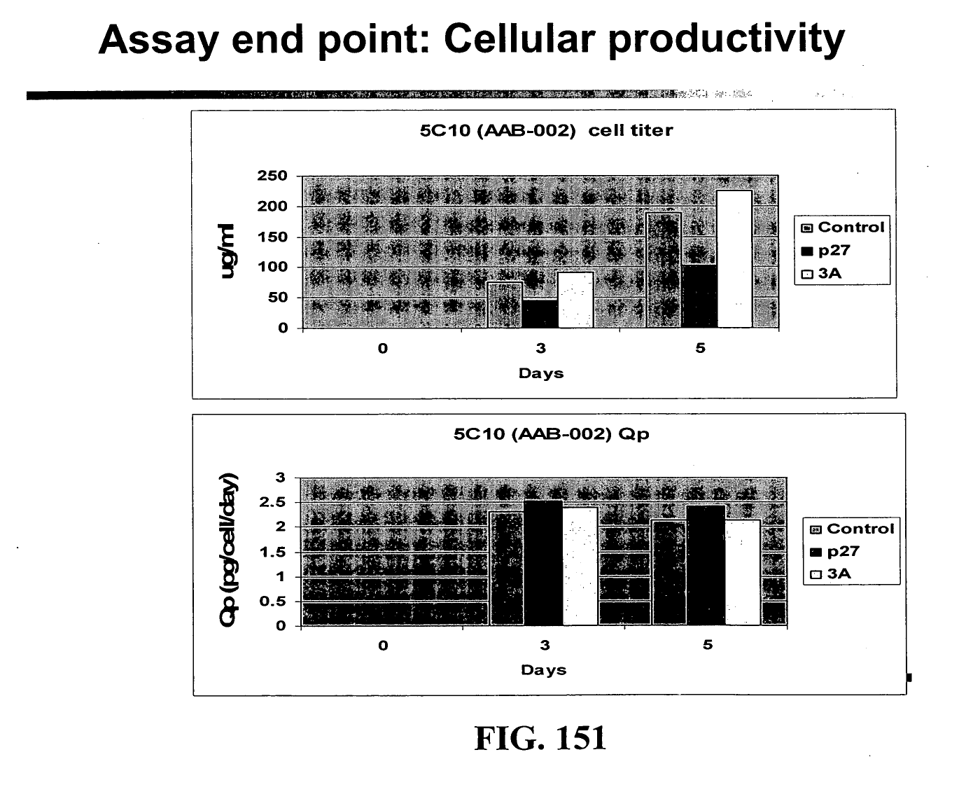

For example, nucleic acids overexpressing specific targets can be introduced into CHO cells by transient transfections and then the impact of over-expression on cellular growth and productivity are monitored. An exemplary protocol, 24 well format, was illustrated in FIGS. 147 and 148.

Growth and productivity controls are typically used for overexpression assays. For example, positive growth/viability control used in this experiment included Ha-Ras and Bcl-xL. Negative growth control used included p27. Other suitable growth and productivity controls are known in the art and can be used for overexpression assays. Additional standard controls such as no nucleic acid control (transfection reagents only) were also included.

Target genes and the control genes were cloned into the pExpress1 vector and introduced into various model cell lines as shown in Table 22.

TABLE 22
Cell lines for the assay and their characteristics

| Clone | Characteristics |
|---|---|
| 1.18 | Middle of the road |
| 5C10 | Middle of the road |
| DE-6 | Middle of the road |
| DD-11 | Middle of the road |
| 1.14 | HCGR, sustained high viability, not |
| | sustained high Qp, Not low NH4, |
| | Not high cell density |
| 2.8 | High max Qp, sustained high Qp |
| 3B12 | High max Qp, sustained high Qp, |
| | high cell density, low lactate |
| DA-4 | Not HCGR, Not high cell density |
| 5B5 | Not sustained high Qp, Not low |
| | lactate |
| 2B6 | Not high cell density, Not high max |
| | Qp |

The 24 well format was used to distinguish phenotypic effects of transient transfection of various genes on various cell lines. Cellular growth and productivity were determined. Exemplary results are illustrated in FIGS. 149-151. It was found that results were generally representative and reproducible. Exemplary overexpression results are summarized in Table 23.

TABLE 23
Summary of the overexpression assays

| Gene over- | Cell line | Growth | Productivity | |
|---|---|---|---|---|
| expressed | tested | phenotype | phenotype | |
| P27 | | | Titer | Qp |
| | 1.14 | ↓ (− − −) | NA | NA |
| | 1.18 | ↓ (−) | NA | NA |
| | 5C10 | ↓ (− −) | ↓ (− −) | ↑ (−) |
| | 5B5 | ↓ (− − −) | ↓ (− −) | ↑ (−) |
| | 3B12 | ↓ (− − −) | NA | NA |
| | 2B6 | ↓ (− − −) | NA | NA |
| | 2.8 | ↓ (−) | NA | NA |
| | DE-6 | ↓ (− −) | NA | NA |
| | DD-11 | ↓ (− −) | NA | NA |
| Bcl-xL | 5B5 | ↑ (+ + +) | ↑ (+ +) | No change |
| H-Ras | 5C10 | ↓ (− −) | ↓ (− −) | No change |
| | 5B5 | ↓ (− − −) | ↓ (− −) | ↓ (−) |
| | 3B12 | ↓ (− −) | NA | NA |
| 3A (EIF4B) | 5C10 | ↑ (+) | ↑ (+) | No change |
| | 5B5 | ↑ (+) | ↑ (+) | No change |

NA = Not tested or awaiting results
(* * *) Strongly increased or decreased
(* *) Increased or decreased
(*) moderately increased or decreased

### Example 13

### Engineering Cell Lines to Improve Cell Phenotypes Based on the Verified Target Genes

The verified target genes are used to effect a cell phenotype, particularly a phenotype characterized by increased and efficient production of a recombinant transgene, increased cell growth rate, high peak cell density, sustained high cell viability, high maximum cellular productivity, sustained high cellular productivity, low ammonium production, and low lactate production, etc. Exemplary target genes are disclosed above, for example, in Tables 2 through 20 and in Tables 24 through 30.

TABLE 24

| | | | | | | | | | | | | |
|---|---|---|---|---|---|---|---|---|---|---|---|---|
| | | | Human | | | | Mouse | | | | | |
| Qualifier List | Symbol | Title | Unigene ID | eValue | % ID | % QC | Unigene ID | eValue | % ID | % QC | FC | Function |

High Cell Growth
Rate

| | | | | | | | | | | | | |
|---|---|---|---|---|---|---|---|---|---|---|---|---|
| U62588_x_at | SDC1 | Syndecan 1 | Hs.224607 | 1E−32 | 93 | 53.2 | Mm.2580 | 7E−48 | 91 | 81.4 | down | Adhesion |
| (SEQ ID NO: 1586) | | | | | | | | | | | | |
| WAN008D2Q_at | Eif4b | Eukaryotic translation initiation factor | #N/A | 6E−49 | 93 | 29.4 | Mm.290022 | 1E−129 | 91 | 71.6 | up | translation (initiation) |
| (SEQ ID NO: 1561) | | 4B (Eif4b) | | | | | | | | | | |
| WAN008DJ9_at | SLC1A4 | Solute carrier family 1 | Hs.323878 | 2E−39 | 87 | 39.5 | Mm.6379 | 1E−121 | 89 | 90.6 | down | serine transporter |
| (SEQ ID NO: 1565) | | (glutamate/neutral amino acid | | | | | | | | | | |
| | | transporter), member 4 | | | | | | | | | | |
| WAN013I0W_at | TAPBP | TAP binding protein (tapasin) | Hs.370937 | 2E−57 | 81 | 93.2 | Mm.154457 | 1E−149 | 87 | 95.3 | down | ER peptide transporter |
| (SEQ ID NO: 1580) | | | | | | | | | | | | |
| WAN013I0X_at | GSS | Glutathione synthetase | Hs.82327 | 3E−96 | 90 | 56.1 | Mm.252316 | 1E−129 | 95 | 55.7 | down | glutathione synthesis |
| (SEQ ID NO: 1581) | | | | | | | | | | | | (protect from oxidative |
| | | | | | | | | | | | | stress) |
| WAN013I1G_at | SLC25A20 | Solute carrier family 25 | Hs.13845 | 1E−137 | 87 | 87.7 | Mm.29666 | 0 | 92 | 86.4 | down | fatty acid translocation |
| (SEQ ID NO: 1582) | | (carnitine/acylcarnitine translocase), | | | | | | | | | | across mitochondrial |
| | | member 20 | | | | | | | | | | membrane |
| WAN013I8K_at | NA | Cluster includes D29972 Cricetulus | #N/A | | | | #N/A | | | | up | |
| (SEQ ID NO: 1584) | | griseus mitochondrial DNA, D-loop | | | | | | | | | | |
| | | region. | | | | | | | | | | |
| X51747_at | HSPB1 | Heat shock 27 kDa protein 1 | Hs.520973 | 1E−101 | 87 | 50.5 | Mm.13849 | 0 | 92 | 66.1 | up | UPR |
| (SEQ ID NO: 1587) | | | | | | | | | | | | |
| WAN008EE0_x_at | Ndufs1 | NADH dehydrogenase (ubiquinone) | Hs.471207 | | | | Mm.290791 | | | | up | electron transport in |
| (SEQ ID NO: 1789) | | Fe—S protein 1 | | | | | | | | | | Mitochondria |

High Cell Density

| | | | | | | | | | | | | |
|---|---|---|---|---|---|---|---|---|---|---|---|---|
| AF022945-rc_f_at | Thbd | Thrombomodulin | Hs.2030 | | | | Mm.24096 | 1E−13 | 90 | 65 | up | thrombin binding |
| (SEQ ID NO: 1666) | | | | | | | | | | | | |
| AF081141_at | CCL2 | Chemokine (C-C motif) ligand 2 | Hs.303649 | 1E−12 | 98 | 9.01 | Mm.290320 | 6E−41 | 91 | 28.1 | down | cytokine; inflammation |
| (SEQ ID NO: 1667) | | | | | | | | | | | | |
| M27838_s_at | ASNS | Asparagine synthetase | Hs.489207 | 0 | 89 | 100 | Mm.2942 | 0 | 92 | 100 | up | aparagine synthesis |
| (SEQ ID NO: 1670) | | | | | | | | | | | | |
| U29167_at | TPM2 | Tropomyosin 2 (beta) | Hs.300772 | 0 | 93 | 88.8 | Mm.646 | 0 | 95 | 90.3 | up | focal adhesion |
| (SEQ ID NO: 1672) | | | | | | | | | | | | |
| WAN0088X2_at | PEO1 | Progressive external ophthalmoplegia 1 | Hs.22678 | 1E−141 | 89 | 94.5 | Mm.105585 | 7E−78 | 91 | 47.8 | up | mitochondrial DNA |
| (SEQ ID NO: 1593) | | | | | | | | | | | | helicase |
| WAN008CQP_at | AATF | Apoptosis antagonizing transcription | Hs.195740 | 6E−73 | 84 | 99.3 | Mm.257482 | 8E−99 | 86 | 99.3 | up | |
| (SEQ ID NO: 1598) | | factor | | | | | | | | | | |
| WAN008CX9_at | ISGF3G | Interferon-stimulated transcription | Hs.1706 | 2E−64 | 83 | 81.5 | Mm.2032 | 1E−119 | 88 | 88.5 | up | |
| (SEQ ID NO: 1599) | | factor 3, gamma 48 kDa | | | | | | | | | | |
| WAN008CXC_at | ATP6V0A1 | ATPase, H+ transporting, lysosomal | Hs.463074 | 0 | 93 | 99.4 | Mm.340818 | 0 | 94 | 100 | down | acidification of |
| (SEQ ID NO: 1600) | | V0 subunit a isoform 1 | | | | | | | | | | intracellular organelles |
| WAN008D2S_at | BPY2IP1 | BPY2 interacting protein 1 | Hs.66048 | 6E−15 | 84 | 21 | Mm.248559 | 1E−101 | 87 | 69 | down | microtubule binding |
| (SEQ ID NO: 1601) | | | | | | | | | | | | |
| WAN008D55-rc_at | LAMB1 | Laminin, beta 1 | Hs.489646 | 1E−155 | 88 | 97.5 | Mm.172674 | 1E−161 | 92 | 77.7 | 1up, | glycoprotein; cell |
| (SEQ ID NO: 1603) | | | | | | | | | | | 1down | adhesion |
| WAN008D5V_x_at | Gosr2 | Golgi SNAP receptor complex | Hs.463278 | | | | Mm.195451 | 1E−08 | 90 | 43.6 | down | transporter; golgi |
| (SEQ ID NO: 1562) | | member 2, mRNA (cDNA clone | | | | | | | | | | trafficking |
| | | MGC: 6437 IMAGE: 3601627) | | | | | | | | | | |
| WAN008D6R_at | TMED4 | Transmembrane emp24 protein | Hs.510745 | 1E−111 | 91 | 73.7 | Mm.254495 | 1E−140 | 92 | 86.6 | down | transporter; unknown |
| (SEQ ID NO: 1604) | | transport domain containing 4 | | | | | | | | | | function |
| WAN008DMI_at | ACSL5 | Acyl-CoA synthetase long-chain family | Hs.11638 | 1E−118 | 85 | 96.6 | #N/A | 0 | 90 | 99.6 | up | lipid biosynthesis; fatty |
| (SEQ ID NO: 1610) | | member 5 | | | | | | | | | | acid degradation |
| WAN008DWJ_at | USP1 | Similar to ubiquitin specific protease 1 | Hs.35086 | 0 | 93 | 97 | Mm.371692 | 0 | 94 | 96.5 | up | de-ubiquitinating |
| (SEQ ID NO: 1614) | | | | | | | | | | | | enzyme |
| WAN008E5L_at | SLC1A5 | Solute carrier famliy 1 (neutral | Hs.515494 | 8E−42 | 84 | 45.6 | Mm.1056 | 1E−115 | 88 | 83.3 | up | amino acid |
| (SEQ ID NO: 1619) | | amino acid transporter), member 5 | | | | | | | | | | transporter |
| WAN008E9N_at | KLHL7 | Kelch-like 7 (Drosophila) | Hs.385861 | 1E−150 | 89 | 99.4 | Mm.273768 | 0 | 93 | 89.1 | down | unknown function |
| (SEQ ID NO: 1620) | | | | | | | | | | | | |
| WAN008EBP_at | Sqstm1 | Sequestosome 1 | Hs.529892 | 0 | 93 | 97.8 | Mm.40828 | 0 | 93 | 97.8 | down | ubiquitin-associated |
| (SEQ ID NO: 1621) | | | | | | | | | | | | protein |
| WAN008EH5_at | PRNP | Prion protein (p27-30) (Creutzfeld- | Hs.472010 | 9E−45 | 87 | 34.8 | Mm.648 | 4E−92 | 90 | 57.2 | down | |
| (SEQ ID NO: 1622) | | Jakob disease, Gerstmann- | | | | | | | | | | |
| | | Strausler-Scheinker Syndrome, | | | | | | | | | | |
| | | fatal familial insomnia) | | | | | | | | | | |
| WAN008ELE_at | PSAT1 | Phosphoserine aminotransferase 1 | Hs.494261 | 7E−27 | 93 | 16.2 | Mm.289936 | 5E−70 | 88 | 58.2 | up | serine biosynthesis |
| (SEQ ID NO: 1626) | | | | | | | | | | | | |
| WAN008EM4_at | ARHGAP18 | Rho GTPase activating protein 18 | Hs.486458 | 1E−109 | 85 | 97.9 | Mm.356496 | 1E−147 | 88 | 100 | down | unknown function |
| (SEQ ID NO: 1627) | | | | | | | | | | | | |
| WAN008EOB_at | NOL1 | Nucleolar protein 1, 120 kDa | Hs.534334 | 8E−48 | 90 | 42.5 | Mm.29203 | 1E−120 | 87 | 92.2 | up | cell cycle progression |
| (SEQ ID NO: 1629) | | | | | | | | | | | | |
| WAN008ERI_at | FNBP3 | Formin binding protein 3 | Hs.298735 | 4E−81 | 97 | 98.9 | Mm.257474 | 2E−94 | 99 | 100 | down | pre-mRNA processing |
| (SEQ ID NO: 1632) | | | | | | | | | | | | |
| WAN008ERP_at | LEPREL1 | Leprecan-like 1 | Hs.374191 | 1E−45 | 87 | 92.8 | Mm.326869 | 1E−68 | 89 | 94 | down | negative regulation of |
| (SEQ ID NO: 1634) | | | | | | | | | | | | cell proliferation(?) |
| WAN008EUO_at | LPL | Lipoprotein lipase | Hs.180878 | 2E−82 | 88 | 74.1 | Mm.1514 | 1E−113 | 92 | 74.1 | down | glycerolipid metabolism |
| (SEQ ID NO: 1635) | | | | | | | | | | | | |
| WAN008F1P_x_at | NA | WAN008F1P 11165A-A01 | #N/A | | | | #N/A | | | | down | Homologs in new array, |
| (SEQ ID NO: 1637) | | | | | | | | | | | | but +/− in most cases, |
| | | | | | | | | | | | | and none of these have |
| | | | | | | | | | | | | any homology |
| WAN013HW0_x_at | NA | Cluster includes WAN008CO3 | #N/A | | | | #N/A | | | | up | Appears to be |
| (SEQ ID NO: 1640) | | 10600D-F02 | | | | | | | | | | mitochondrial |
| | | | | | | | | | | | | polycistronic mRNA!!! |
| WAN013HX8_f_at | EIF4A2 | Eukaryotic translation initiation factor | Hs.478553 | 1E−155 | 96 | 100 | Mm.260084 | 1E−155 | 96 | 100 | down | translation initiation |
| (SEQ ID NO: 1490) | | 4A, isoform 2 | | | | | | | | | | |
| WAN013I1U_x_at | NA | Cluster includes WAN008BLL | #N/A | 2E−05 | 92 | 7.71 | #N/A | 1E−05 | 92 | 7.71 | up | |
| (SEQ ID NO: 1648) | | 11233C-H10 | | | | | | | | | | |
| WAN013I2T_at | CBX5 | Chromobox homolog 5 (HP1 alpha | Hs.349283 | 1E−142 | 92 | 72 | Mm.262059 | 1E−168 | 95 | 72 | up | chromatin binding |
| (SEQ ID NO: 1652) | | homolog, Drosophila) | | | | | | | | | | |
| WAN013I6J_s_at | CAD | Carbamoyl-phosphate synthetase 2, | Hs.377010 | 0 | 91 | 99.5 | Mm.305535 | 0 | 94 | 99.5 | up | pyrimidine biosynthesis |
| (SEQ ID NO: 1657) | | aspartate transcarbamylase, and | | | | | | | | | | |
| | | dihydroorotase | | | | | | | | | | |
| WAN013I8X_at | HSPD1 | Heat shock 60 kDa protein 1 | Hs.113684 | 0 | 90 | 99.8 | Mm.1777 | 0 | 93 | 99.8 | up | molecular chaperone |
| (SEQ ID NO: 1661) | | (chaperonin) | | | | | | | | | | |
| WANO13I9Z_at | GNAS | guanine nucleotide binding protein, | Hs.125898 | | | | Mm.125770 | 0 | 94 | 41.1 | down | cell growth |
| (SEQ ID NO: 1664) | | alpha stimulating | | | | | | | | | | |
| WAN013I9F_at | HSPA9B | Heat shock protein 9A | Hs.184233 | 3E−29 | 92 | 18.7 | Mm.209419 | 2E−72 | 91 | 44.8 | up | cell proliferation |
| (SEQ ID NO: 1662) | | | | | | | | | | | | |

High Max Qp

| | | | | |
|---|---|---|---|---|
| gi\|34853001 | Uap1l1 | PREDICTED: similar to UDP-N- | Mm.33797 | −2.22 |
| | | acteylglucosamine pyrophosphorylase 1-like 1 | | |

Sustained High Cell
Viability

| | | | | | | | | | | | |
|---|---|---|---|---|---|---|---|---|---|---|---|
| AF022942_at | Cirbp | Cold inducible RNA binding protein | Hs.634522 | 8E−40 | 86 | 86.5 | Mm.17898 | 9E−94 | 95 | 100 | up |
| (SEQ ID NO: 2008) | | | | | | | | | | | |
| AF120325_f_at | TUBB2B | Tubulin, beta 2B | Hs.300701 | 0 | 89 | 72.7 | #N/A | 0 | 92 | 78.5 | up |
| (SEQ ID NO: 1753) | | | | | | | | | | | |
| M12329_at | NA | M12329 Chinese hamster alpha- | #N/A | 0 | 93 | 54.3 | #N/A | 0 | 96 | 53.8 | up |
| (SEQ ID NO: 1942) | | tubulin III mRNA, complete cds. | | | | | | | | | |
| M96676_at | LGALS1 | Lectin, galactoside-binding, soluble, 1 | Hs.445351 | 1E−122 | 89 | 100 | Mm.43831 | 1E−131 | 90 | 100 | up |
| (SEQ ID NO: 1727) | | (galectin 1) | | | | | | | | | |
| WAN0088YL_f_at | 2700085E05Rik | RIKEN cDNA 2700085E05 gene | #N/A | 2E−58 | 91 | 96.5 | Mm.249700 | 4E−77 | 94 | 100 | up |
| (SEQ ID NO: 2009) | | | | | | | | | | | |
| WAN008CZP_at | NA | WAN008CZP 10604A-A08 | #N/A | 3E−29 | 91 | 21.4 | #N/A | 7E−50 | 83 | 62.5 | up |
| (SEQ ID NO: 1962) | | | | | | | | | | | |
| WAN008E65_at | ERP29 | Endoplasmic reticulum protein 29 | Hs.75841 | 1E−164 | 91 | 79.8 | Mm.154570 | 1E−171 | 91 | 83.1 | up |
| (SEQ ID NO: 1976) | | | | | | | | | | | |
| WAN008940_at | MRPL37 | Mitochondrial ribosomal protein L37 | Hs.584908 | 5E−68 | 86 | 54.6 | Mm.29517 | 1E−102 | 90 | 60.7 | down |
| (SEQ ID NO: 2010) | | Glioma tumor suppressor candidate | | | | | | | | | |
| WAN008CQI_at | GLTSCR2 | region gene 2 | Hs.421907 | 1E−113 | 86 | 100 | Mm.277634 | 1E−175 | 91 | 100 | down |
| (SEQ ID NO: 2011) | | | | | | | | | | | |
| WAN008DAG_at | AARS | Alanyl-tRNA synthetase | Hs.315137 | 1E−101 | 89 | 70.7 | Mm.24174 | 1E−134 | 92 | 74.7 | down |
| (SEQ ID NO: 2012) | | | | | | | | | | | |
| WAN008DSH_at | MRPL16 | Mitochondrial ribosomal protein L16 | Hs.530734 | 1E−37 | 85 | 40 | Mm.203928 | 4E−64 | 90 | 42.4 | down |
| (SEQ ID NO: 2013) | | | | | | | | | | | |
| WAN008DXE_x_at | SLC6A8 | Solute carrier family 6 | Hs.540696 | 1E−59 | 95 | 99.3 | Mm.274553 | 1E−64 | 97 | 99.3 | down |
| (SEQ ID NO: 2014) | | (neurotransmitter transporter, | | | | | | | | | |
| | | creatine), member 8 | | | | | | | | | |
| WAN008E2E_at | PSMC4 | Proteasome (prosome, macropain) | Hs.211594 | 1E−153 | 92 | 100 | Mm.29582 | 1E−141 | 91 | 100 | down |
| (SEQ ID NO: 1567) | | 26S subunit, ATPase, 4 | | | | | | | | | |
| WAN008E5L_at | SLC1A5 | Solute carrier family 1 (neutral amino | Hs.631582 | 8E−42 | 84 | 45.6 | Mm.1056 | 1E−115 | 88 | 83.3 | down |
| (SEQ ID NO: 1619) | | acid transporter), member 5 | | | | | | | | | |
| WAN008EE3_at | SARS | Seryl-tRNA synthetase | Hs.531176 | 1E−116 | 91 | 100 | Mm.28688 | 1E−136 | 93 | 100 | down |
| (SEQ ID NO: 1809) | | | | | | | | | | | |
| WAN013HVH_at | GARS | Glycyl-tRNA synthetase | Hs.404321 | 1E−177 | 88 | 100 | Mm.250004 | 0 | 94 | 100 | down |
| (SEQ ID NO: 2015) | | | | | | | | | | | |
| WAN013I1O_at | RNH1 | Ribonuclease/angiogenin inhibitor 1 | Hs.530687 | 1E−18 | 83 | 27.8 | Mm.279485 | 1E−91 | 88 | 61.2 | down |
| (SEQ ID NO: 1804) | | | | | | | | | | | |
| WAN013I1Q_at | TXNL2 | Thioredoxin-like 2 | Hs.42644 | 2E−90 | 89 | 53.5 | Mm.267692 | 1E−126 | 94 | 53.8 | down |
| (SEQ ID NO: 2016) | | | | | | | | | | | |
| WAN008EE5_at | PANX1 | Pannexin 1 | Hs.591976 | 1E−125 | 89 | 100 | Mm.142253 | 1E−177 | 94 | 100 | down |
| (SEQ ID NO: 2017) | | | | | | | | | | | |

Sustained High Qp

| | | | | | | | | | | | |
|---|---|---|---|---|---|---|---|---|---|---|---|
| WAN013HUM_at | EHD4 | EH-domain containing 4 | Hs.143703 | 1E−95 | 93 | 59 | Mm.132226 | 1E−125 | 89 | 99.5 | up |
| (SEQ ID NO: 1576) | | | | | | | | | | | |
| AB014875_at | PLS3 | plastin 3 (T isoform) | Hs.496622 | 1E−159 | 92 | 31.6 | Mm.28777 | 1E−175 | 90 | 40.7 | down |
| (SEQ ID NO: 1734) | | | | | | | | | | | |
| WAN0088PY_at | NA | WAN0088PY 10595D-B07 | Hs.279806 | 0 | 0 | 0 | Mm.220038 | 2E−07 | 93 | 9.23 | down |
| (SEQ ID NO: 1480) | | | | | | | | | | | |
| WAN008CIU_at | NA | WAN008CIU 10599D-C10 | #N/A | 0 | 0 | 0 | #N/A | 1E−05 | 100 | 5.79 | down |
| (SEQ ID NO: 2018) | | | | | | | | | | | |
| WAN008EYO_at | NA | WAN008EYO 11233B-B05 | #N/A | 0 | 0 | 0 | #N/A | 3E−09 | 89 | 12.4 | down |
| (SEQ ID NO: 2019) | | | | | | | | | | | |
| WAN008CIA_at | EIF1AY | Eukaryotic translation initiation factor | Hs.461178 | 1E−137 | 91 | 72 | Mm.294623 | 1E−164 | 89 | 99.6 | up |
| (SEQ ID NO: 2020) | | 1A, Y-linked | | | | | | | | | |
| WAN008D6O_at | STRBP | Spermatid perinuclear RNA binding | Hs.645506 | 9E−62 | 90 | 67.7 | Mm.237095 | 3E−94 | 95 | 71 | up |
| (SEQ ID NO: 1692) | | protein | | | | | | | | | |
| WAN008DNJ_at | Rbmxrt | RNA binding motif protein, X | Hs.380118 | 1E−131 | 92 | 71 | Mm.24718 | 0 | 95 | 97.9 | up |
| (SEQ ID NO: 2021) | | chromosome retrogene | | | | | | | | | |
| WAN008DWF_f_at | NA | WAN008DWF 11229A-H02 | #N/A | 0 | 0 | 0 | #N/A | 0 | 0 | 0 | up |
| (SEQ ID NO: 2022) | | | | | | | | | | | |
| WAN013HUG_at | CDKN2C | Cyclin-dependent kinase inhibitor 2C | Hs.525324 | 1E−113 | 95 | 53.7 | Mm.1912 | 1E−142 | 99 | 53.1 | up |
| (SEQ ID NO: 2023) | | (p18, inhibits CDK4) | | | | | | | | | |
| WAN0088OY_x_at | HNRPF | Heterogeneous nuclear | Hs.558477 | 1E−87 | 99 | 95.8 | Mm.317706 | 1E−98 | 100 | 100 | down |
| (SEQ ID NO: 2024) | | ribonucleoprotein F | | | | | | | | | |
| WAN013I8H_x_at | APP | Amyloid beta (A4) precursor protein | Hs.642685 | 1E−77 | 84 | 83.5 | Mm.277585 | 1E−167 | 88 | 100 | down |
| (SEQ ID NO: 1548) | | (protease nexin-II, Alzheimer disease) | | | | | | | | | |
| X53074_f_at | HPRT1 | Hypoxanthine | Hs.412707 | 5E−35 | 93 | 47.4 | Mm.299381 | 7E−36 | 93 | 47.8 | down |
| (SEQ ID NO: 2025) | | phosphoribosyltransferase 1 (Lesch- | | | | | | | | | |
| | | Nyhan syndrome) | | | | | | | | | |
| WAN008EJ7_at | EIF5A | Eukaryotic translation initiation factor | Hs.534314 | 0 | 99 | 100 | Mm.196607 | 0 | 98 | 100 | down |
| (SEQ ID NO: 2026) | | 5A | | | | | | | | | |

Low Ammonia
Producer

| | | | | | | | | | | | |
|---|---|---|---|---|---|---|---|---|---|---|---|
| AF180918_at | KLHL5 | Kelch-like 5 (Drosophila) | Hs.272251 | 6E−21 | 89 | 19.8 | Mm.10281 | 5E−48 | 86 | 49.2 | up |
| (SEQ ID NO: 1778) | | | | | | | | | | | |
| WAN013HW0_x_at | NA | Cluster includes WAN008CO3 | #N/A | 0 | 0 | 0 | #N/A | 0 | 0 | 0 | up |
| (SEQ ID NO: 1640) | | 10600D-F02 | | | | | | | | | |
| WAN013I8U_at | NA | Cluster includes M14311 Chinese | #N/A | 2E−05 | 92 | 20.3 | #N/A | 0 | 0 | 0 | up |
| (SEQ ID NO: 2027) | | Hamster mitochondrial ATPase 6 and | | | | | | | | | |
| | | URF A6L genes, complete cds. | | | | | | | | | |
| AF100738_at | SUI1 | Putative translation initiation factor | #N/A | 8E−09 | 85 | 53.4 | #N/A | 2E−11 | 89 | 30.8 | down |
| (SEQ ID NO: 2028) | | | | | | | | | | | |
| L00176_at | Hmgcr | 3-hydroxy-3-methylglutaryl-Coenzyme | Hs.643495 | 7E−54 | 88 | 57.9 | Mm.316652 | 3E−82 | 94 | 55.4 | down |
| (SEQ ID NO: 1500) | | A reductase | | | | | | | | | |
| L00334_at | Hmgcs1 | 3-hydroxy-3-methylglutaryl-Coenzyme | Hs.397729 | 1E−100 | 88 | 36 | Mm.61526 | 1E−178 | 90 | 45.5 | down |
| (SEQ ID NO: 2029) | | A synthase 1 | | | | | | | | | |
| M29238_at | DDIT3 | DNA-damage-inducible transcript 3 | Hs.505777 | 1E−100 | 87 | 76.8 | Mm.110220 | 1E−119 | 89 | 68.9 | down |
| (SEQ ID NO: 2030) | | | | | | | | | | | |
| M60973_at | GADD45A | Growth arrest and DNA-damage- | Hs.80409 | 0 | 92 | 76.3 | Mm.389750 | 1E−170 | 91 | 49.2 | down |
| (SEQ ID NO: 2031) | | inducible, alpha | | | | | | | | | |
| U29660_s_at | NA | U29660 Cricetulus griseus hydrogen | #N/A | 0 | 0 | 0 | #N/A | 1E−13 | 85 | 8.15 | down |
| (SEQ ID NO: 2032) | | peroxide-inducible adapt33A RNA. | | | | | | | | | |
| U48852_at | CRELD2 | Cysteine-rich with EGF-like domains 2 | Hs.211282 | 1E−109 | 82 | 55.1 | Mm.292567 | 0 | 89 | 91.7 | down |
| (SEQ ID NO: 1502) | | | | | | | | | | | |
| U67146_at | EEF1E1 | Eukaryotic translation elongation | Hs.631818 | 1E−152 | 89 | 63.9 | Mm.36683 | 0 | 90 | 90.4 | down |
| (SEQ ID NO: 1683) | | factor 1 epsilon 1 | | | | | | | | | |
| WAN0088II_at | BNIP2 | BCL2/adenovirus E1B 19 kDa | Hs.283454 | 1E−154 | 89 | 90.3 | Mm.159777 | 0 | 94 | 96 | down |
| (SEQ ID NO: 2033) | | interacting protein 2 | | | | | | | | | |
| WAN0088Z9_at | PLAA | Phospholipase A2-activating protein | Hs.27182 | 1E−148 | 88 | 100 | Mm.22724 | 0 | 94 | 99 | down |
| (SEQ ID NO: 2034) | | | | | | | | | | | |
| WAN008BRV_at | NA | WAN008BRV 11231C-E04 | #N/A | 1E−49 | 94 | 30.1 | #N/A | 8E−99 | 95 | 55.6 | down |
| (SEQ ID NO: 2035) | | | | | | | | | | | |
| WAN008BT4_at | NA | WAN008BT4 11231C-A04 | #N/A | 1E−45 | 85 | 84.1 | #N/A | 8E−64 | 86 | 89 | down |
| (SEQ ID NO: 2036) | | | | | | | | | | | |
| WAN008CF7_at | IVNS1ABP | Influenza virus NS1A binding protein | Hs.497183 | 0 | 93 | 100 | Mm.33764 | 0 | 97 | 100 | down |
| (SEQ ID NO: 1889) | | | | | | | | | | | |
| WAN008CLU_at | Emp1 | Epithelial membrane protein 1 | Hs.436298 | 0 | 0 | 0 | Mm.182785 | 3E−28 | 90 | 21.7 | down |
| (SEQ ID NO: 1953) | | | | | | | | | | | |
| WAN008CPJ_at | Fdft1 | Farnesyl diphosphate farnesyl | Hs.593928 | 1E−123 | 85 | 99.6 | Mm.425927 | 0 | 0 | 0 | down |
| (SEQ ID NO: 1878) | | transferase 1 | | | | | | | | | |
| WAN008CTB_at | TIPRL | TIP41, TOR signalling pathway | Hs.209431 | 3E−69 | 91 | 37.7 | Mm.21520 | 1E−134 | 90 | 73.9 | down |
| (SEQ ID NO: 2037) | | regulator-like (S. cerevisiae) | | | | | | | | | |
| WAN008CVX_at | CDC20 | CDC20 cell division cycle 20 homolog | Hs.524947 | 1E−169 | 91 | 85.2 | Mm.289747 | 0 | 92 | 87.3 | down |
| (SEQ ID NO: 1958) | | (S. cerevisiae) | | | | | | | | | |
| WAN008CW2_at | SRP54 | Signal recognition particle 54 kDa | Hs.167535 | 0 | 92 | 99.6 | Mm.12848 | 0 | 93 | 99.8 | down |
| (SEQ ID NO: 2038) | | | | | | | | | | | |
| WAN008D31_at | Lss | Lanosterol synthase | Hs.596543 | 1E−80 | 85 | 68.3 | Mm.55075 | 1E−150 | 92 | 74 | down |
| (SEQ ID NO: 1964) | | | | | | | | | | | |
| WAN008D4O_at | NA | WAN008D4O 10604D-C05 | #N/A | 0 | 0 | 0 | #N/A | 2E−12 | 93 | 15.3 | down |
| (SEQ ID NO: 2039) | | | | | | | | | | | |
| WAN008DGF_x_at | Ddx5 | DEAD (Asp-Glu-Ala-Asp) box | Hs.279806 | 6E−23 | 96 | 77.9 | Mm.220038 | 1E−34 | 97 | 100 | down |
| (SEQ ID NO: 2040) | | polypeptide 5 | | | | | | | | | |
| WAN008DUZ_at | POP7 | Processing of precursor 7, | Hs.416994 | 6E−52 | 90 | 35.2 | Mm.290242 | 9E−62 | 92 | 35.2 | down |
| (SEQ ID NO: 1689) | | ribonuclease P subunit (S. cerevisiae) | | | | | | | | | |
| WAN008E0W_at | LMAN2 | Lectin, mannose-binding 2 | Hs.75864 | 7E−67 | 87 | 53.8 | Mm.38868 | 1E−130 | 89 | 88.4 | down |
| (SEQ ID NO: 2041) | | | | | | | | | | | |
| WAN008E3R_at | DDX41 | DEAD (Asp-Glu-Ala-Asp) box | Hs.484288 | 1E−176 | 89 | 99.3 | Mm.205045 | 0 | 95 | 100 | down |
| (SEQ ID NO: 2042) | | polypeptide 41 | | | | | | | | | |
| WAN008E4Z_at | Nup153 | Nucleoporin 153 | Hs.601591 | 1E−169 | 90 | 92.5 | Mm.255398 | 0 | 94 | 99.1 | down |
| (SEQ ID NO: 1975) | | | | | | | | | | | |
| WAN008EED_at | Sc5d | Sterol-C5-desaturase (fungal ERG3, | Mm.32700 | 2E−42 | 85 | 40.7 | Mm.32700 | 9E−99 | 88 | 70 | down |
| (SEQ ID NO: 1521) | | delta-5-desaturase) homolog (S. cerevisae) | | | | | | | | | |
| WAN008EXX_at | CCT4 | Chaperonin containing TCP1, subunit | Hs.421509 | 1E−48 | 89 | 55 | Mm.296985 | 3E−54 | 89 | 59.1 | down |
| (SEQ ID NO: 2043) | | 4 (delta) | | | | | | | | | |
| WAN008EXZ_at | NA | WAN008EXZ 11233B-E01 | #N/A | 0 | 0 | 0 | #N/A | 4E−67 | 86 | 68.3 | down |
| (SEQ ID NO: 2044) | | | | | | | | | | | |
| WAN013HUI_at | HIP2 | Huntingtin interacting protein 2 | Hs.50308 | 0 | 97 | 94.8 | Mm.319512 | 0 | 97 | 99.2 | down |
| (SEQ ID NO: 2045) | | | | | | | | | | | |
| WAN013HX4_at | ESD | Esterase D/formylglutathione | Hs.432491 | 1E−158 | 88 | 93.5 | Mm.38055 | 0 | 92 | 93.2 | down |
| (SEQ ID NO: 1578) | | hydrolase | | | | | | | | | |
| WAN013HY6_at | IARS | Isoleucine-tRNA synthetase | Hs.445403 | 1E−127 | 86 | 100 | Mm.21118 | 0 | 92 | 99.2 | down |
| (SEQ ID NO: 2046) | | | | | | | | | | | |
| WAN013HYE_at | Psmd11_predicted | Proteasome (prosome, macropain) | #N/A | 0 | 92 | 100 | #N/A | 0 | 94 | 100 | down |
| (SEQ ID NO: 2047) | | 26S subunit, non-ATPase, 11 | | | | | | | | | |
| | | (predicted) | | | | | | | | | |
| WAN013I88_at | NA | Cluster includes AF003836 | #N/A | 0 | 0 | 0 | #N/A | 8E−32 | 87 | 14 | down |
| (SEQ ID NO: 2048) | | Mesocricetus auratus isopentenyl | | | | | | | | | |
| | | diphosphate:dimethylallyl diphosphate | | | | | | | | | |
| | | isomerase mRNA, complete cds. | | | | | | | | | |
| WAN013I8A_at | NA | Cluster includes AF044676 Cricetulus | #N/A | 0 | 0 | 0 | #N/A | 7E−38 | 86 | 33.3 | down |
| (SEQ ID NO: 2049) | | griseus glucose-6-phosphate | | | | | | | | | |
| | | dehydrogenase mRNA, complete cds. | | | | | | | | | |
| WAN013I8W_at | Hspa5 | Heat shock 70 kD protein 5 (glucose- | Hs.605502 | 0 | 93 | 95.6 | Mm.330160 | 0 | 97 | 100 | down |
| (SEQ ID NO: 2050) | | regulated protein) | | | | | | | | | |
| WAN013I9H_at | HSP90B1 | Heat shock protein 90 kDa beta | Hs.192374 | 1E−104 | 89 | 58.5 | Mm.87773 | 1E−120 | 91 | 59 | down |
| (SEQ ID NO: 2051) | | (Grp94), member 1 | | | | | | | | | |
| WAN013IAD_at | TOP2A | Topoisomerase (DNA) II alpha | Hs.156346 | 3E−37 | 81 | 29.5 | Mm.4237 | 1E−86 | 84 | 37.6 | down |
| (SEQ ID NO: 2005) | | 170 kDa | | | | | | | | | |
| AF221841_at | Prdx1 | Peroxiredoxin 1 | Hs.180909 | 0 | 91 | 100 | Mm.30929 | 0 | 94 | 98.5 | up |
| (SEQ ID NO: 1735) | | | | | | | | | | | |
| WAN008DI8_at | Lmna | Lamin A | #N/A | 0 | 0 | 0 | Mm.243014 | 0 | 93 | 99.8 | up |
| (SEQ ID NO: 2052) | | | | | | | | | | | |
| WAN008DXT_at | SUCLA2 | Succinate-CoA ligase, ADP-forming, | Hs.546323 | 2E−54 | 94 | 34.7 | Mm.38951 | 4E−60 | 96 | 33 | up |
| (SEQ ID NO: 2053) | | beta subunit | | | | | | | | | |

Low Lactate
Producer

| | | | | | | | | | | | |
|---|---|---|---|---|---|---|---|---|---|---|---|
| AF022942_at | Cirbp | Cold inducible RNA binding protein | Hs.634522 | 8E−40 | 86 | 86.5 | Mm.17898 | 9E−94 | 95 | 100 | up |
| (SEQ ID NO: 2008) | | | | | | | | | | | |
| M26640_at | CLU | Clusterin | Hs.436657 | 7E−92 | 83 | 94.6 | Mm.200608 | 0 | 92 | 98.8 | up |
| (SEQ ID NO: 2054) | | | | | | | | | | | |
| WAN0088OY_x_at | Invs | Inversin | Hs.558477 | 1E−87 | 99 | 95.8 | Mm.317706 | 1E−98 | 100 | 100 | down |
| (SEQ ID NO: 2024) | | | | | | | | | | | |
| WAN008CLU_at | Emp1 | Epithelial membrane protein 1 | Hs.436298 | 0 | 0 | 0 | Mm.182785 | 3E−28 | 90 | 21.7 | down |
| (SEQ ID NO: 1953) | | | | | | | | | | | |
| WAN008EED_at | Sc5d | Sterol-C5-desaturase (fungal ERG3, | #N/A | 2E−42 | 85 | 40.7 | Mm.32700 | 9E−99 | 88 | 70 | down |
| (SEQ ID NO: 1521) | | delta-5-desaturase) homolog (S. cerevisae) | | | | | | | | | |
| L00332_at | HMGCS1 | 3-hydroxy-3-methylglutaryl-Coenzyme | Hs.397729 | 3E−41 | 91 | 95.8 | Mm.61526 | 1E−42 | 92 | 88.9 | down |
| (SEQ ID NO: 1862) | | A synthase 1 (soluble) | | | | | | | | | |

Multiple Categories

| | | | | | | | | | | | | |
|---|---|---|---|---|---|---|---|---|---|---|---|---|
| WAN0088K2_at | DUSP16 | Dual specificity phosphatase 16 | Hs.536535 | 2E−05 | 84 | 16 | Mm.3994 | 4E−21 | 88 | 22.7 | down | |
| (SEQ ID NO: 1945) | | | | | | | | | | | | |
| WAN0088JV_at | TRIB3 | Tribbles homolog 3 (Drosophila) | Hs.516826 | 4E−62 | 82 | 86.4 | Mm.276018 | 1E−158 | 89 | 98.1 | down | |
| (SEQ ID NO: 1555) | | | | | | | | | | | | |
| WAN0088OP_at | Hrb2 | HIV-1 Rev binding protein 2 | #N/A | 1E−141 | 88 | 91.2 | #N/A | 0 | 91 | 99.4 | down | |
| (SEQ ID NO: 2055) | | | | | | | | | | | | |
| WAN008BSH_at | CAT | Catalase | Hs.502302 | 6E−16 | 89 | 38.7 | Mm.4215 | 3E−41 | 89 | 85.7 | down | reactive oxygen |
| (SEQ ID NO: 1558) | | | | | | | | | | | | species |
| WAN008BSL_at | PAK1IP1 | PAK1 interacting protein 1 | Hs.310231 | 1E−109 | 90 | 86.1 | Mm.24789 | 4E−91 | 86 | 99.7 | down | |
| (SEQ ID NO: 2056) | | | | | | | | | | | | |
| WAN008CIU_at | NA | WAN008CIU 10599D-C10 | #N/A | 0 | 0 | 0 | #N/A | 1E−05 | 100 | 5.79 | down | |
| (SEQ ID NO: 2018) | | | | | | | | | | | | |
| WAN008CZ6_at | 2010106G01Rik | RIKEN cDNA 2010106G01 gene | #N/A | 1E−125 | 86 | 99.2 | Mm.269928 | 1E−164 | 89 | 100 | down | |
| (SEQ ID NO: 2057) | | | | | | | | | | | | |
| WAN008E89_at | Nup160 | Nucleoporin 160 | Hs.645358 | 0 | 92 | 98.1 | Mm.24532 | 0 | 94 | 99.8 | down | |
| (SEQ ID NO: 1900) | | | | | | | | | | | | |
| WAN008EC4_at | HIAT1 | Hippocampus abundant transcript 1 | Hs.124156 | 1E−140 | 93 | 66 | Mm.280077 | 1E−155 | 95 | 66 | down | |
| (SEQ ID NO: 2058) | | | | | | | | | | | | |
| WAN013HX1_at | SEC13L1 | SEC13-like 1 (S. cerevisiae) | Hs.166924 | 0 | 90 | 99.3 | Mm.29296 | 0 | 90 | 99.3 | down | |
| (SEQ ID NO: 2059) | | | | | | | | | | | | |
| WAN013HXZ_x_at | NA | Cluster includes WAN008DCG | #N/A | 6E−22 | 91 | 46.1 | #N/A | 5E−30 | 97 | 30.6 | down | |
| (SEQ ID NO: 2060) | | 11165C-D11 | | | | | | | | | | |
| WAN013I05_at | Abcb6 | ATP-binding cassette, sub-family B | Hs.107911 | 1E−154 | 87 | 100 | Mm.28663 | 0 | 92 | 100 | down | |
| (SEQ ID NO: 1646) | | (MDR/TAP), member 6 | | | | | | | | | | |
| WAN013I0B_at | NA | Cluster includes WAN008E5C | #N/A | 3E−07 | 83 | 15.6 | #N/A | 6E−22 | 86 | 17.1 | down | |
| (SEQ ID NO: 2061) | | 11230A-B11 | | | | | | | | | | |
| WAN013I1D_x_at | CCNDBP1 | Cyclin D-type binding-protein 1 | Hs.36794 | 2E−67 | 79 | 87 | Mm.7838 | 1E−111 | 82 | 95.1 | down | |
| (SEQ ID NO: 2062) | | | | | | | | | | | | |
| WAN013I1Z_f_at | NA | Cluster includes WAN0088KD | #N/A | 0 | 0 | 0 | #N/A | 0 | 0 | 0 | up | |
| (SEQ ID NO: 2063) | | 10595B-H02 | | | | | | | | | | |
| WAN013I20_x_at | MAFG | V-maf musculoaponeurotic | Hs.252229 | 7E−39 | 83 | 30.8 | Mm.268010 | 2E−50 | 82 | 42.7 | down | |
| (SEQ ID NO: 2064) | | fibrosarcoma oncogene homolog G | | | | | | | | | | |
| | | (avian) | | | | | | | | | | |
| WAN013I31_at | RNF4 | Ring finger protein 4 | Hs.66394 | 8E−67 | 90 | 47.8 | Mm.21281 | 1E−178 | 92 | 95.4 | down | |
| (SEQ ID NO: 2065) | | | | | | | | | | | | |
| X51747_at | HSPB1 | Heat shock 27 kDa protein 1 | Hs.520973 | 1E−101 | 87 | 50.5 | Mm.13849 | 0 | 92 | 66.1 | up | |
| (SEQ ID NO: 1587) | | | | | | | | | | | | |
| WAN008DGD_at | Aplp2 | Amyloid beta (A4) precursor-like | #N/A | | | | Mm.19133 | 7E−69 | 93 | 44.5 | −1.3 | migration; adhesion |
| (SEQ ID NO: 1564) | | protein 2 (Aplp2) | | | | | | | | | | |
| U42430_at | CD36 | CD36 antigen (collagen type I | Hs.120949 | 6E−43 | 86 | 34.3 | Mm.18628 | 2E−57 | 88 | 37.7 | −1.49 | |
| (SEQ ID NO: 1673) | | receptor, thrombospondin receptor) | | | | | | | | | | |
| L00181_at | Hmgcr | 3-hydroxy-3-methylglutaryl-Coenzyme | Hs.643495 | 1E−37 | 90 | 32.7 | Mm.316652 | 3E−52 | 94 | 33.7 | down | |
| (SEQ ID NO: 1510) | | A reductase | | | | | | | | | | |
| U22819_s_at | SREBF2 | Sterol regulatory element binding | Hs.443258 | 1E−118 | 90 | 99.5 | Mm.38016 | 1E−133 | 92 | 97 | down | |
| (SEQ ID NO: 1927) | | transcription factor 2 | | | | | | | | | | |
| L00366_x_at | TK1 | Thymidine kinase 1, soluble | Hs.515122 | 4E−18 | 90 | 84.9 | Mm.2661 | 1E−16 | 89 | 86 | up | |
| (SEQ ID NO: 1941) | | | | | | | | | | | | |

TABLE 25
High Priority Secondary Gene list

| | | | | Human | |
|---|---|---|---|---|---|
| Qualifier List | Category | Symbol | Title | Unigene ID | eValue |
| AB020230_at | HCD | INTRON | delta-sarcoglycan intron 1 | #N/A | 3E−08 |
| (SEQ ID NO: 1665) | DAG | | | | |
| AF113614_at | HCD | TLR2 | Toll-like receptor 2 | Hs.519033 | 1E−101 |
| (SEQ ID NO: 1668) | DAG | | | | |
| AF320819_at | HCD | BSG | Basigin (OK blood group) | Hs.501293 | 1E−25 |
| (SEQ ID NO: 1669) | DAG | | | | |
| M76730_at | HCD | Col5a1 | Procollagen, type V, alpha 1 | Hs.210283 | 6E−52 |
| (SEQ ID NO: 1671) | DAG | | | | |
| U62588_x_at | HCD | SDC1 | Syndecan 1 | Hs.224607 | 1E−32 |
| (SEQ ID NO: 1586) | DAG | | | | |
| WAN0088J9_x_at | HCD | CCNA2 | cyclin A2 | Hs.85137 | 1.00E−55 |
| (SEQ ID NO: 1588) | DAG | | | | |
| WAN008BRK_at | HCD | Tmsb4x | Thymosin, beta 4, X | Hs.522584 | 1E−153 |
| (SEQ ID NO: 1594) | DAG | | chromosome | | |
| WAN008BSG_x_at | HCD | TRAM1 | Translocation associated | Hs.491988 | 7E−29 |
| (SEQ ID NO: 1595) | DAG | | membrane protein 1 | | |
| WAN008CHP_x_at | HCD | NA | WAN008CHP 10599D-H02 | #N/A | |
| (SEQ ID NO: 1596) | DAG | | | | |
| WAN008D3Z_at | HCD | GALNT7 | UDP-N-acetyl-alpha-D- | Hs.127407 | 1E−135 |
| (SEQ ID NO: 1602) | DAG | | galactosamine: polypeptide N- | | |
| | | | acetylgalactosaminyltransferase | | |
| | | | 7 (GalNAc-T7) | | |
| WAN008DFT_at | HCD | ABHD6 | Abhydrolase domain containing 6 | Hs.476454 | 3E−17 |
| (SEQ ID NO: 1605) | DAG | | | | |
| WAN008DI7_at | HCD | FBXO42 | F-box protein 42 | #N/A | |
| (SEQ ID NO: 1607) | DAG | | | | |
| WAN008DIA_at | HCD | U2AF1 | U2(RNU2) small nuclear RNA | Hs.365116 | 1E−170 |
| (SEQ ID NO: 1608) | DAG | | auxiliary factor 1 | | |
| WAN008DMJ_at | HCD | NAB2 | NGFI-A binding protein 2 (EGR1 | Hs.159223 | 1E−176 |
| (SEQ ID NO: 1611) | DAG | | binding protein 2) | | |
| WAN008DS9_at | HCD | CFL2 | Cofilin 2 (muscle) | Hs.180141 | 1E−113 |
| (SEQ ID NO: 1613) | DAG | | | | |
| WAN008E06_at | HCD | Rabep2 | Rabaptin, RAB GTPase binding | Hs.555978 | 2E−92 |
| (SEQ ID NO: 1616) | DAG | | effector protein 2 | | |
| WAN008EKK_at | HCD | PSMA8 | Proteasome (prosome, | Hs.464813 | 1E−104 |
| (SEQ ID NO: 1625) | DAG | | macropain) subunit, alpha type, 8 | | |
| WAN008END_at | HCD | SCYL1 | SCY1-like 1 (S. cerevisiae) | Hs.238839 | 2E−61 |
| (SEQ ID NO: 1628) | DAG | | | | |
| WAN008EQM_at | HCD | NA | RC WAN008EQM 11232D-D11 | #N/A | |
| (SEQ ID NO: 1630) | DAG | | | | |
| WAN008ERO_at | HCD | SNAG1 | Sorting nexin associated golgi | Hs.432755 | 7E−30 |
| (SEQ ID NO: 1633) | DAG | | protein 1 | | |
| WAN008EY0_at | HCD | C330017I15Rik | RIKEN cDNA C330017I15 gene | HS.520619 | 1E−179 |
| (SEQ ID NO: 1636) | DAG | | | | |
| WAN013HVJ_at | HCD | Rn.75246 | Similar to RIKEN cDNA | #N/A | 7E−78 |
| (SEQ ID NO: 1638) | DAG | | 2310045A20 | | |
| WAN013HZK_at | HCD | NA | Cluster includes WAN008DS2 | #N/A | |
| (SEQ ID NO: 1643) | DAG | | 11228C-H04 | | |
| WAN013HZP_at | HCD | Eif4g2 | Eukaryotic translation initiation | Hs.183684 | 1E−179 |
| (SEQ ID NO: 1644) | DAG | | factor 4, gamma 2 | | |
| WAN013I01_at | HCD | MCFD2 | Multiple coagulation factor | Hs.293689 | 4E−59 |
| (SEQ ID NO: 1645) | DAG | | deficiency 2 | | |
| WAN013I15_at | HCD | SUCLG2 | Succinate-CoA ligase, GDP- | Hs.186512 | 1E−157 |
| (SEQ ID NO: 1647) | DAG | | forming, beta subunit | | |
| WAN013I2F_at | HCD | THBD | Thrombomodulin | Hs.2030 | 1E−18 |
| (SEQ ID NO: 1649) | DAG | | | | |
| WAN013I2K_at | HCD | TMEFF1 | Transmembrane protein with | Hs.336224 | 8E−93 |
| (SEQ ID NO: 1650) | DAG | | EGF-like and two follistatin-like | | |
| | | | domains 1 | | |
| WAN013I6C_at | HCD | SLC16A1 | Solute carrier family 16 | Hs.75231 | 2E−26 |
| (SEQ ID NO: 1655) | DAG | | (monocarboxylic acid | | |
| | | | transporters), member 1 | | |
| WAN013I6E_x_at | HCD | GSTP1 | Glutathione S-transferase pi | Hs.523836 | 1E−129 |
| (SEQ ID NO: 1656) | DAG | | | | |
| WAN013I8B_at | HCD | Akr1a4 | Aldo-keto reductase family 1, | Hs.474584 | 0 |
| (SEQ ID NO: 1659) | DAG | | member A4 (aldehyde | | |
| | | | reductase) | | |
| WAN013I8V_at | HCD | NCL | Nucleolin | Hs.79110 | 1E−111 |
| (SEQ ID NO: 1660) | DAG | | | | |
| WAN013I9G_at | HCD | SLC3A2 | Solute carrier family 3 (activators | Hs.502769 | 1E−105 |
| (SEQ ID NO: 1663) | DAG | | of dibasic and neutral amino acid | | |
| | | | transport), member 2 | | |
| WAN0088PR_at | HCD | CCPG1 | Cell cycle progression 1 | Hs.612814 | 2E−08 |
| (SEQ ID NO: 1589) | DCU | | | | |
| WAN0088Q6_at | HCD | Hist1h2bn | Histone 1, H2bn | Hs.534368 | 1E−153 |
| (SEQ ID NO: 1590) | DCU | | | | |
| WAN0088S8_at | HCD | SLC29A1 | Solute carrier family 29 | Hs.25450 | 3E−35 |
| (SEQ ID NO: 1591) | DCU | | (nucleoside transporters), | | |
| | | | member 1 | | |
| WAN0088T2_at | HCD | ATF4 | Activating transcription factor 4 | Hs.496487 | 1E−158 |
| (SEQ ID NO: 1592) | DCU | | (tax-responsive enhancer | | |
| | | | element B67) | | |
| WAN008CM7_x_at | HCD | MRPL51 | Mitochondrial ribosomal protein | Hs.55847 | 0.0002 |
| (SEQ ID NO: 1597) | DCU | | L51 | | |
| WAN008DGZ_at | HCD | SLC7A6OS | Solute carrier family 7, member | Hs.334848 | 2E−79 |
| (SEQ ID NO: 1606) | DCU | | 6 opposite strand | | |
| WAN008DJ8_f_at | HCD | Ubc | Ubiquitin C, mRNA (cDNA clone | Hs.378821 | 1E−22 |
| (SEQ ID NO: 1609) | DCU | | IMAGE: 2645223) | | |
| WAN008DQE_at | HCD | YES1 | V-yes-1 Yamaguchi sarcoma | Hs.194148 | 0 |
| (SEQ ID NO: 1612) | DCU | | viral oncogene homolog 1 | | |
| WAN008E2Q_at | HCD | GSPT1 | G1 to S phase transition 1 | Hs.528780 | 0 |
| (SEQ ID NO: 1618) | DCU | | | | |
| WAN008EJY_at | HCD | NA | WAN008EJY 11232A-H04 | #N/A | |
| (SEQ ID NO: 1624) | DCU | | | | |
| WAN008ERB_at | HCD | PCBP1 | Poly(rC) binding protein 1 | Hs.2853 | 0 |
| (SEQ ID NO: 1631) | DCU | | | | |
| WAN013HVL_at | HCD | UGDH | UDP-glucose dehydrogenase | Hs.28309 | 1E−160 |
| (SEQ ID NO: 1639) | DCU | | | | |
| WAN013HZ3_at | HCD | ARMCX3 | Armadillo repeat containing, X- | Hs.172788 | 4E−09 |
| (SEQ ID NO: 1642) | DCU | | linked 3 | | |
| WAN013I2L_at | HCD | SLC7A5 | solute carrier family 7 (cationic | Hs.513797 | 9.00E−07 |
| (SEQ ID NO: 1651) | DCU | | amino acid transporter, y+ | | |
| | | | system), member 5 | | |
| WAN013I3P_at | HCD | CAMLG | Calcium modulating ligand | Hs.529846 | 1E−147 |
| (SEQ ID NO: 1653) | DCU | | | | |
| WAN013I61_at | HCD | Nppb | Natriuretic peptide precursor | Hs.219140 | |
| (SEQ ID NO: 1654) | DCU | | type B | | |
| WAN013I6P_x_at | HCD | ABCB1 | ATP-binding cassette, sub-family | Hs.489033 | 0 |
| (SEQ ID NO: 1658) | DCU | | B (MDR/TAP), member 1 | | |
| Y00365_at | HCD | HMGB1 | High-mobility group box 1 | Hs.434102 | 1E−102 |
| (SEQ ID NO: 1674) | DCU | | | | |
| AF081143_at | HCGR | RPS18 | Ribosomal protein S18 | Hs.546290 | 1E−78 |
| (SEQ ID NO: 1585) | DAG | | | | |
| WAN0088PT_at | HCGR | Psmc1 | Protease (prosome, macropain) | Hs.356654 | 0 |
| (SEQ ID NO: 1556) | DAG | | 26S subunit, ATPase 1 | | |
| WAN0088XH_at | HCGR | HERPUD1 | Homocysteine-inducible, | Hs.146393 | 7E−79 |
| (SEQ ID NO: 1557) | DAG | | endoplasmic reticulum stress- | | |
| | | | inducible, ubiquitin-like domain | | |
| | | | member 1 | | |
| WAN008CM1_x_at | HCGR | NA | WAN008CM1 106000-F11 | WAN008CM1_x_at | #N/A |
| (SEQ ID NO: 1559) | DAG | | | Blast Report | |
| WAN008D6J_at | HCGR | HMGA2 | High mobility group AT-hook 2 | Hs.505924 | 4E−62 |
| (SEQ ID NO: 1563) | DAG | | | | |
| WAN008DSE_at | HCGR | SLC1A4 | Solute carrier family 1 | Hs.323878 | 2E−82 |
| (SEQ ID NO: 1566) | DAG | | (glutamate/neutral amino acid | | |
| | | | transporter), member 4 | | |
| WAN008EBJ_at | HCGR | Triobp | TRIO and F-actin binding protein | Hs.533030 | 6E−89 |
| (SEQ ID NO: 1569) | DAG | | | | |
| WAN008EFS_at | HCGR | TXNRD1 | Thioredoxin reductase 1 | Hs.567352 | 6E−18 |
| (SEQ ID NO: 1570) | DAG | | | | |
| WAN008EGV_at | HCGR | GDI2 | GDP dissociation inhibitor 2 | Hs.299055 | 0 |
| (SEQ ID NO: 1571) | DAG | | | | |
| WAN008EMQ_at | HCGR | KPNA3 | Karyopherin alpha 3 (importin | Hs.527919 | 1E−144 |
| (SEQ ID NO: 1572) | DAG | | alpha 4) | | |
| WAN008ERL_at | HCGR | ETFA | Electron transferring | Hs.39925 | 1E−135 |
| (SEQ ID NO: 1573) | DAG | | flavoprotein, alpha polypeptide | | |
| | | | (Etfa), nuclear gene encoding | | |
| | | | mitochondrial protein, mRNA | | |
| WAN008ETP_at | HCGR | AADACL1 | Arylacetamide deacetylase-like 1 | Hs.444099 | 2E−72 |
| (SEQ ID NO: 1574) | DAG | | | | |
| WAN008EX2_x_at | HCGR | IFRD1 | Interferon-related developmental | Hs.7879 | 7E−39 |
| (SEQ ID NO: 1575) | DAG | | regulator 1 | | |
| WAN013HUM_at | HCGR | EHD4 | EH-domain containing 4 | Hs.143703 | 1E−95 |
| (SEQ ID NO: 1576) | DAG | | | | |
| WAN013HWG_at | HCGR | NA | Cluster includes WAN008CSP | WAN013HWG_at | #N/A |
| (SEQ ID NO: 1577) | DAG | | 10602B-D10 | Blast Report | |
| WAN013HYO_at | HCGR | RPL11 | Ribosomal protein L11 | Hs.388664 | 0 |
| (SEQ ID NO: 1579) | DAG | | | | |
| WAN013I38_at | HCGR | Pkm2 | Pyruvate kinase, muscle, mRNA | Hs.198281 | 3E−63 |
| (SEQ ID NO: 1583) | DAG | | (cDNA clone MGC: 11908 | | |
| | | | IMAGE: 3598842) | | |
| WAN008CWC_x_at | HCGR | NA | WAN008CWC 10603C-F10 | #N/A | |
| (SEQ ID NO: 1560) | DCU | | | | |
| WAN008E8M_at | HCGR | HADHB | Hydroxyacyl-Coenzyme A | Hs.515848 | 1E−114 |
| (SEQ ID NO: 1568) | DCU | | dehydrogenase/3-ketoacyl- | | |
| | | | Coenzyme A thiolase/enoyl- | | |
| | | | Coenzyme A hydratase | | |
| | | | (trifunctional protein), beta | | |
| | | | subunit | | |
| U48852_at | HMQP | CRELD2 | Cysteine-rich with EGF-like | Hs.211282 | 1E−109 |
| (SEQ ID NO: 1502) | DAG | | domains 2 | | |
| WAN013HX9_at | HMQP | Cnbp1 | Cellular nucleic acid binding | Hs.518249 | 0 |
| (SEQ ID NO: 2066) | DAG | | protein 1 (Cnbp1), mRNA | | |
| WAN013HZ1_at | HMQP | MRLC2 | Myosin regulatory light chain | Hs.464472 | 1E−161 |
| (SEQ ID NO: 2067) | DAG | | MRLC2 | | |
| WAN013166_f_at | HMQP | Vim | Vimentin (Vim), mRNA | Hs.533317 | 1E−126 |
| (SEQ ID NO: 1494) | DAG | | | | |
| WAN013IAB_x_at | HMQP | TP53 | Tumor protein p53 (Li-Fraumeni | Hs.408312 | 1E−150 |
| (SEQ ID NO: 1496) | DAG | | syndrome) | | |
| WAN0088OD_at | HMQP | Pnrc1 | Proline-rich nuclear receptor | Hs.75969 | |
| (SEQ ID NO: 2068) | DCU | | coactivator 1 | | |
| WAN0088OT_at | HMQP | NA | WAN0088OT 10595D-F11 | #N/A | |
| (SEQ ID NO: 1479) | DCU | | | | |
| WAN0088U4_at | HMQP | C21orf66 | Chromosome 21 open reading | Hs.473635 | |
| (SEQ ID NO: 2069) | DCU | | frame 66 | | |
| WAN008DRM_at | HMQP | EPHX1 | Hypothetical gene supported by | Hs.89649 | |
| (SEQ ID NO: 1503) | DCU | | AK124699 | | |
| WAN008EEK_at | HMQP | NA | WAN008EEK 11231A-B10 | #N/A | |
| (SEQ ID NO: 2070) | DCU | | | | |
| WAN008F2S_at | HMQP | NA | WAN008F2S 11165A-F02 | #N/A | |
| SEQ ID NO: 1489 | DCU | | | | |
| AF115410_s_at | LAP DAG | DPM2 | Dolichyl-phosphate | Hs.108973 | 1E−38 |
| (SEQ ID NO: 2071) | Post TS | | mannosyltransferase polypeptide | | |
| | | | 2, regulatory subunit | | |
| AF157566_at | LAP DAG | GNAT1 | Guanine nucleotide binding | Hs.517978 | 1E−107 |
| (SEQ ID NO: 2072) | Post TS | | protein (G protein), alpha | | |
| | | | transducing activity polypeptide 1 | | |
| WAN0088SH_at | LAP DAG | GPR177 | G protein-coupled receptor 177 | Hs.22137 | 1E−172 |
| (SEQ ID NO: 2073) | Post TS | | | | |
| WAN0088Y2_at | LAP DAG | Ggnbp2 | Gametogenetin binding protein 2 | #N/A | 0 |
| (SEQ ID NO: 1882) | Post TS | | | | |
| WAN0088ZI_at | LAP DAG | PPP2R2A | Protein phosphatase 2 (formerly | Hs.146339 | 0 |
| (SEQ ID NO: 2074) | Post TS | | 2A), regulatory subunit B (PR | | |
| | | | 52), alpha isoform | | |
| WAN0088ZJ_at | LAP DAG | Slc4a2 | Solute carrier family 4 (anion | #N/A | 1E−162 |
| (SEQ ID NO: 2075) | Post TS | | exchanger), member 2 | | |
| WAN00896L_f_at | LAP DAG | NA | WAN00896L 10599C-A02 | #N/A | 0 |
| (SEQ ID NO: 2076) | Post TS | | | | |
| WAN008BNG_at | LAP DAG | LRRC28 | Leucine rich repeat containing | Hs.578684 | 4E−90 |
| (SEQ ID NO: 1780) | Post TS | | 28 | | |
| WAN008CI5_at | LAP DAG | CDC20 | CDC20 cell division cycle 20 | Hs.524947 | 1E−105 |
| (SEQ ID NO: 1839) | Post TS | | homolog (S. cerevisiae) | | |
| WAN008CRQ_at | LAP DAG | NA | WAN008CRQ 10602B-H08 | #N/A | 0 |
| (SEQ ID NO: 2077) | Post TS | | | | |
| WAN008CRX_at | LAP DAG | SNX13 | Sorting nexin 13 | Hs.585343 | 1E−175 |
| (SEQ ID NO: 2078) | Post TS | | | | |
| WAN008D4W_at | LAP DAG | PPT2 | Palmitoyl-protein thioesterase 2 | Hs.332138 | 6E−82 |
| (SEQ ID NO: 2079) | Post TS | | | | |
| WAN008DGZ_at | LAP DAG | NA | WAN008DGZ 11188B-H07 | #N/A | 2E−79 |
| (SEQ ID NO: 1606) | Post TS | | | | |
| WAN008DJI_at | LAP DAG | DARS | Aspartyl-tRNA synthetase | Hs.503787 | 2E−82 |
| (SEQ ID NO: 2080) | Post TS | | | | |
| WAN008DKS_at | LAP DAG | MAPK8IP1 | Mitogen-activated protein kinase | Hs.234249 | 1E−116 |
| (SEQ ID NO: 1787) | Post TS | | 8 interacting protein 1 | | |
| WAN008DL6_at | LAP DAG | Rn.6896 | Transcribed locus, strongly | #N/A | 1E−86 |
| (SEQ ID NO: 2081) | Post TS | | similar to XP_219519.2 | | |
| | | | PREDICTED: similar to C184L | | |
| | | | ORF1 protein [Rattus | | |
| | | | norvegicus] | | |
| WAN008DWE_at | LAP DAG | Nxf1 | Nuclear RNA export factor 1 | #N/A | 3E−15 |
| (SEQ ID NO: 2082) | Post TS | | homolog (S. cerevisiae) | | |
| WAN008E71_at | LAP DAG | ITCH | Itchy homolog E3 ubiquitin | Hs.632272 | 2E−97 |
| (SEQ ID NO: 2083) | Post TS | | protein ligase (mouse) | | |
| WAN008E8M_at | LAP DAG | HADHB | Hydroxyacyl-Coenzyme A | Hs.534639 | 1E−114 |
| (SEQ ID NO: 1568) | Post TS | | dehydrogenase/3-ketoacyl- | | |
| | | | Coenzyme A thiolase/enoyl- | | |
| | | | Coenzyme A hydratase | | |
| | | | (trifunctional protein), beta | | |
| | | | subunit | | |
| WAN008EDZ_at | LAP DAG | MSH6 | MutS homolog 6 (E. coli) | Hs.445052 | 1E−124 |
| (SEQ ID NO: 2084) | Post TS | | | | |
| WAN008EHM_at | LAP DAG | CLU | Clusterin | Hs.436657 | 1E−71 |
| (SEQ ID NO: 1709) | Post TS | | | | |
| WAN008EIX_at | LAP DAG | NA | WAN008EIX 11231D-C08 | #N/A | 0 |
| (SEQ ID NO: 2085) | Post TS | | | | |
| WAN008EP8_at | LAP DAG | TMEM39A | Transmembrane protein 39A | Hs.434927 | 4E−52 |
| (SEQ ID NO: 2086) | Post TS | | | | |
| WAN008EXG_at | LAP DAG | NUP98 | Nucleoporin 98 kDa | Hs.524750 | 1E−108 |
| (SEQ ID NO: 1901) | Post TS | | | | |
| WAN013HV6_x_at | LAP DAG | NA | Cluster includes WAN008F0U | #N/A | 3E−09 |
| (SEQ ID NO: 2087) | Post TS | | 10599A-F06 | | |
| WAN013HV8_x_at | LAP DAG | NA | Cluster includes WAN008F17 | #N/A | 2E−32 |
| (SEQ ID NO: 2088) | Post TS | | 10599A-G07 | | |
| WAN013HW2_at | LAP DAG | PSMC6 | Proteasome (prosome, | Hs.156171 | 0 |
| (SEQ ID NO: 2089) | Post TS | | macropain) 26S subunit, | | |
| | | | ATPase, 6 | | |
| WAN13HZ6_at | LAP DAG | BRD2 | Bromodomain containing 2 | Hs.75243 | 1E−122 |
| (SEQ ID NO: 2090) | Post TS | | | | |
| WAN013HZJ_at | LAP DAG | YY1 | YY1 transcription factor | Hs.388927 | 0 |
| (SEQ ID NO: 2091) | Post TS | | | | |
| WAN013I0C_at | LAP DAG | Cacybp | Calcyclin binding protein | Hs.447653 | 1E−153 |
| (SEQ ID NO: 2092) | Post TS | | | | |
| WAN013I3P_at | LAP DAG | CAMLG | Calcium modulating ligand | Hs.529846 | 1E−147 |
| (SEQ ID NO: 1653) | Post TS | | | | |
| WAN013I43_at | LAP DAG | FAU | Finkel-Biskis-Reilly murine | Hs.387208 | 1E−119 |
| (SEQ ID NO: 2093) | Post TS | | sarcoma virus (FBR-MuSV) | | |
| | | | ubiquitously expressed (fox | | |
| | | | derived); ribosomal protein S30 | | |
| WAN013I8C_at | LAP DAG | B3GAT3 | Beta-1,3-glucuronyltransferase 3 | Hs.449191 | 1E−159 |
| (SEQ ID NO: 2094) | Post TS | | (glucuronosyltransferase 1) | | |
| WAN008DT7_at | LAP DAG | GSTO1 | Glutathione S-transferase | Hs.190028 | 5E−65 |
| (SEQ ID NO: 1486) | PreTS | | omega 1 | | |
| WAN008EKU_at | LAP DAG | TAX1BP1 | Tax1 (human T-cell leukemia | Hs.34576 | 1E−120 |
| (SEQ ID NO: 1741) | PreTS | | virus type I) binding protein 1 | | |
| WAN013I4A_at | LAP DAG | CBR3 | Carbonyl reductase 3 | Hs.154510 | 0 |
| (SEQ ID NO: 2095) | PreTS | | | | |
| AF242536_at | LAP DCU | CSNK1E | Casein kinase 1, epsilon | Hs.474833 | 0 |
| (SEQ ID NO: 2096) | Post TS | | | | |
| AJ223076_at | LAP DCU | NA | AJ223076 Cricetulus griseus | #N/A | 9E−10 |
| (SEQ ID NO: 2097) | Post TS | | mRNA for TRIP protein | | |
| WAN008CTZ_at | LAP DCU | PGD | Phosphogluconate | Hs.464071 | 1E−105 |
| (SEQ ID NO: 2098) | Post TS | | dehydrogenase | | |
| WAN008D16_at | LAP DCU | PIAS1 | Protein inhibitor of activated | Hs.162458 | 1E−128 |
| (SEQ ID NO: 2099) | Post TS | | STAT, 1 | | |
| WAN008DCP_at | LAP DCU | TBC1D10A | TBC1 domain family, member | Hs.444950 | 0 |
| (SEQ ID NO: 2100) | Post TS | | 10A | | |
| WAN008DIE_at | LAP DCU | RAI14 | Retinoic acid induced 14 | Hs.431400 | 6E−95 |
| (SEQ ID NO: 2101) | Post TS | | | | |
| WAN008DKD_at | LAP DCU | MAFG | V-maf musculoaponeurotic | Hs.252229 | 1E−139 |
| (SEQ ID NO: 2102) | Post TS | | fibrosarcoma oncogene homolog | | |
| | | | G (avian) | | |
| WAN008DMP_at | LAP DCU | EWSR1 | Ewing sarcoma breakpoint | Hs.374477 | 1E−157 |
| (SEQ ID NO: 1968) | Post TS | | region 1 | | |
| WAN008DOG_at | LAP DCU | NA | WAN008DOG 11228A-E07 | #N/A | 0 |
| (SEQ ID NO: 2103) | Post TS | | | | |
| WAN008E2D_at | LAP DCU | WNK4 | WNK lysine deficient protein | Hs.105448 | 1E−31 |
| (SEQ ID NO: 2104) | Post TS | | kinase 4 | | |
| WAN008ECD_at | LAP DCU | NARG1 | NMDA receptor regulated 1 | Hs.555985 | 0 |
| (SEQ ID NO: 1898) | Post TS | | | | |
| WAN008EGD_at | LAP DCU | NA | WAN008EGD 11231B-D08 | #N/A | 0 |
| (SEQ ID NO: 2105) | Post TS | | | | |
| WAN008ERJ_x_at | LAP DCU | NA | WAN008ERJ 11232D-A07 | #N/A | 0 |
| (SEQ ID NO: 2106) | Post TS | | | | |
| WAN008ET2_at | LAP DCU | GSS | Glutathione synthetase | Hs.82327 | 5E−77 |
| (SEQ ID NO: 2107) | Post TS | | | | |
| WAN008EVI-rc_at | LAP DCU | Pparbp | Peroxisome proliferator activated | Hs.643754 | 5E−35 |
| (SEQ ID NO: 1902) | Post TS | | receptor binding protein | | |
| WAN008F32_at | LAP DCU | NA | WAN008F32 11165A-G04 | #N/A | 0 |
| (SEQ ID NO: 2108) | Post TS | | | | |
| WAN013HVM_at | LAP DCU | NA | Cluster includes WAN008CJJ | #N/A | 0 |
| (SEQ ID NO: 2109) | Post TS | | 10600B-H12 | | |
| WAN013HVV_at | LAP DCU | GHITM | Growth hormone inducible | Hs.352656 | 1E−148 |
| (SEQ ID NO: 2110) | Post TS | | transmembrane protein | | |
| WAN013HWQ_x_at | LAP DCU | Snx25 | Sorting nexin 25 | Hs.369091 | 1E−28 |
| (SEQ ID NO: 2111) | Post TS | | | | |
| WAN013HY1_at | LAP DCU | ACAT1 | Acetyl-Coenzyme A | Hs.232375 | 1E−161 |
| (SEQ ID NO: 2112) | Post TS | | acetyltransferase 1 (acetoacetyl | | |
| | | | Coenzyme A thiolase) | | |
| WAN013I0A_x_at | LAP DCU | PRPF38B | PRP38 pre-mRNA processing | Hs.342307 | 4E−84 |
| (SEQ ID NO: 2113) | Post TS | | factor 38 (yeast) domain | | |
| | | | containing B | | |
| WAN013I26_at | LAP DCU | IDH3A | Isocitrate dehydrogenase 3 | Hs.591110 | 0 |
| (SEQ ID NO: 2114) | Post TS | | (NAD+) alpha | | |
| WAN013I30_at | LAP DCU | HSP90B1 | Heat shock protein 90 kDa beta | Hs.192374 | 0 |
| (SEQ ID NO: 1528) | Post TS | | (Grp94), member 1 | | |
| WAN013I3A_at | LAP DCU | NA | Cluster includes WAN008C6M | #N/A | 2E−79 |
| (SEQ ID NO: 2115) | Post TS | | 10596B-B05 | | |
| WAN013I8P_at | LAP DCU | LAMP2 | Lysosomal-associated | Hs.496684 | 1E−133 |
| (SEQ ID NO: 1728) | Post TS | | membrane protein 2 | | |
| X53077_x_at | LAP DCU | NA | X53077 C. longicaudatus HPRT | #N/A | 0 |
| (SEQ ID NO: 2116) | Post TS | | gene, exon 5 | | |
| AF121895_at | LAP DCU | NA | AF121895 Cricetulus griseus | #N/A | 0 |
| (SEQ ID NO: 2117) | PreTS | | dolichol-phosphate-mannose | | |
| | | | synthase (DPM1) mRNA, | | |
| | | | complete cds. | | |
| WAN008BNY_at | LAP DCU | NSMCE1 | Non-SMC element 1 homolog | Hs.284295 | 1E−129 |
| (SEQ ID NO: 1791) | PreTS | | (S. cerevisiae) | | |
| WAN008CNN_at | LAP DCU | NUDT9 | Nudix (nucleoside diphosphate | Hs.149500 | 1E−147 |
| (SEQ ID NO: 2118) | PreTS | | linked moiety X)-type motif 9 | | |
| WAN008EOG_at | LAP DCU | NA | WAN008EOG 11232C-D07 | #N/A | 0 |
| (SEQ ID NO: 2119) | PreTS | | | | |
| WAN013HYK_at | LAP DCU | EPS8 | Epidermal growth factor receptor | Hs.591160 | 2E−15 |
| (SEQ ID NO: 1716) | PreTS | | pathway substrate 8 | | |
| Y12837_at | LAP DCU | Fxr1h | Fragile X mental retardation | #N/A | 0 |
| (SEQ ID NO: 2120) | PreTS | | gene 1, autosomal homolog | | |
| AF004831_at | LLP DAG | SPTLC1 | Serine palmitoyltransferase, long | Hs.90458 | 1E−18 |
| (SEQ ID NO: 1536) | | | chain base subunit 1 | | |
| AF072727_at | LLP DAG | JTV1 | JTV1 gene | Hs.301613 | 8E−79 |
| (SEQ ID NO: 2121) | | | | | |
| AF081142_at | LLP DAG | UBA52 | Ubiquitin A-52 residue ribosomal | Hs.5308 | 8E−75 |
| (SEQ ID NO: 2122) | | | protein fusion product 1 | | |
| D86467_at | LLP DAG | TM4SF1 | Transmembrane 4 L six family | Hs.351316 | 2E−41 |
| (SEQ ID NO: 2123) | | | member 1 | | |
| M12329_g_at | LLP DAG | Mm.392113 | Transcribed locus, moderately | #N/A | 1E−132 |
| (SEQ ID NO: 1942) | | | similar to XP_426592.1 | | |
| | | | PREDICTED: similar to tubulin, | | |
| | | | alpha 2; tubulin alpha 2 [Gallus | | |
| | | | gallus] | | |
| M22350_at | LLP DAG | ATP5I | ATP synthase, H+ transporting, | Hs.85539 | 1E−35 |
| (SEQ ID NO: 2124) | | | mitochondrial F0 complex, | | |
| | | | subunit E | | |
| U49841_at | LLP DAG | Gosr1 | Golgi SNAP receptor complex | Hs.645262 | 2E−17 |
| (SEQ ID NO: 2125) | | | member 1 | | |
| WAN0088K3_at | LLP DAG | NA | WAN0088K3 10595A-A03 | #N/A | 0 |
| (SEQ ID NO: 2126) | | | | | |
| WAN0088MM_at | LLP DAG | NA | WAN0088MM 10595C-G09 | #N/A | 0 |
| (SEQ ID NO: 2127) | | | | | |
| WAN0088PU_at | LLP DAG | Ywhab | Tyrosine 3- | Hs.645387 | 1E−102 |
| (SEQ ID NO: 2128) | | | monooxygenase/tryptophan 5- | | |
| | | | monooxygenase activation | | |
| | | | protein, beta polypeptide | | |
| WAN0088X9_at | LLP DAG | RAB34 | RAB34, member RAS oncogene | Hs.301853 | 1E−108 |
| (SEQ ID NO: 1553) | | | family | | |
| WAN0088Z7_at | LLP DAG | GPSN2 | Glycoprotein, synaptic 2 | Hs.515642 | 0 |
| (SEQ ID NO: 2129) | | | | | |
| WAN008900_at | LLP DAG | 2610301K12Rik | RIKEN cDNA 2610301K12 gene | #N/A | 0 |
| (SEQ ID NO: 2130) | | | | | |
| WAN008CN4_at | LLP DAG | NA | WAN008CN4 10600C-B03 | #N/A | 0 |
| (SEQ ID NO: 2131) | | | | | |
| WAN008CS2_at | LLP DAG | VKORC1L1 | Vitamin K epoxide reductase | Hs.427232 | 1E−168 |
| (SEQ ID NO: 1694) | | | complex, subunit 1-like 1 | | |
| WAN008CSN_at | LLP DAG | OACT5 | O-acyltransferase (membrane | #N/A | 1E−140 |
| (SEQ ID NO: 2132) | | | bound) domain containing 5 | | |
| WAN008D3X_at | LLP DAG | PPT1 | Palmitoyl-protein thioesterase 1 | Hs.3873 | 1E−101 |
| (SEQ ID NO: 2133) | | | (ceroid-lipofuscinosis, neuronal | | |
| | | | 1, infantile) | | |
| WAN008DBL_at | LLP DAG | NDUFB9 | NADH dehydrogenase | Hs.15977 | 1E−120 |
| (SEQ ID NO: 1730) | | | (ubiquinone) 1 beta subcomplex, | | |
| | | | 9, 22 kDa | | |
| WAN008DK1_at | LLP DAG | UQCRC1 | Ubiquinol-cytochrome c | Hs.119251 | 3E−69 |
| (SEQ ID NO: 1829) | | | reductase core protein I | | |
| WAN008DLQ_at | LLP DAG | ATP5O | ATP synthase, H+ transporting, | Hs.409140 | 4E−53 |
| (SEQ ID NO: 2134) | | | mitochondrial F1 complex, O | | |
| | | | subunit (oligomycin sensitivity | | |
| | | | conferring protein) | | |
| WAN008DNO_at | LLP DAG | ELP4 | Elongation protein 4 homolog (S. cerevisiae) | Hs.175534 | 1E−126 |
| (SEQ ID NO: 2135) | | | | | |
| WAN008DRQ_x_at | LLP DAG | Sdha | Succinate dehydrogenase | Hs.440475 | 3E−09 |
| (SEQ ID NO: 2136) | | | complex, subunit A, flavoprotein | | |
| | | | (Fp) | | |
| WAN008DXB_f_at | LLP DAG | NA | WAN008DXB 11229A-D08 | #N/A | 0 |
| (SEQ ID NO: 2137) | | | | | |
| WAN008E1P_at | LLP DAG | Nomo1 | Nodal modulator 1 | Hs.583391 | 6E−83 |
| (SEQ ID NO: 2138) | | | | | |
| WAN008E8D_at | LLP DAG | Ipo7 | Importin 7 | Hs.643522 | 1E−136 |
| (SEQ ID NO: 2139) | | | | | |
| WAN008EEE_at | LLP DAG | DDX3X | DEAD (Asp-Glu-Ala-Asp) box | Hs.380774 | 1E−138 |
| (SEQ ID NO: 2140) | | | polypeptide 3, X-linked | | |
| WAN008EH6_at | LLP DAG | STT3A | STT3, subunit of the | Hs.504237 | 0 |
| (SEQ ID NO: 2141) | | | oligosaccharyltransferase | | |
| | | | complex, homolog A (S. cerevisiae) | | |
| WAN008EHM_at | LLP DAG | CLU | Clusterin | Hs.436657 | 1E−71 |
| (SEQ ID NO: 1709) | | | | | |
| WAN008EK5- | LLP DAG | NA | RC WAN008EK5 11232A-G08 | #N/A | 3E−35 |
| rc_x_at | | | | | |
| (SEQ ID NO: 1979) | | | | | |
| WAN008EKV_at | LLP DAG | INSIG1 | Insulin induced gene 1 | Hs.520819 | 1E−83 |
| (SEQ ID NO: 2142) | | | | | |
| WAN008EN3_at | LLP DAG | Cldnd1 | Claudin domain containing 1 | Hs.531371 | 0 |
| (SEQ ID NO: 2142) | | | | | |
| WAN008EOR_x_at | LLP DAG | NA | WAN008EOR 11232C-C06 | #N/A | 0 |
| (SEQ ID NO: 2144) | | | | | |
| WAN008EPF_at | LLP DAG | TXNDC4 | Thioredoxin domain containing 4 | Hs.591899 | 1E−152 |
| (SEQ ID NO: 2145) | | | (endoplasmic reticulum) | | |
| WAN008EPP_at | LLP DAG | Rdh11 | Retinol dehydrogenase 11 | Hs.226007 | 0 |
| (SEQ ID NO: 2146) | | | | | |
| WAN008ERQ_at | LLP DAG | ACTR6 | ARP6 actin-related protein 6 | Hs.115088 | 2E−74 |
| (SEQ ID NO: 2147) | | | homolog (yeast) | | |
| WAN008ESB_at | LLP DAG | NA | WAN008ESB 11233A-F09 | #N/A | 1E−15 |
| (SEQ ID NO: 2148) | | | | | |
| WAN008ESS_at | LLP DAG | NA | WAN008ESS 11233A-D10 | #N/A | 0 |
| (SEQ ID NO: 2149) | | | | | |
| WAN008ET4_at | LLP DAG | ATP6V1E1 | ATPase, H+ transporting, | Hs.517338 | 1E−173 |
| (SEQ ID NO: 2150) | | | lysosomal 31 kDa, V1 subunit E1 | | |
| WAN008EWS_at | LLP DAG | PLAA | Phospholipase A2-activating | Hs.27182 | 0 |
| (SEQ ID NO: 1688) | | | protein | | |
| WAN008F30_f_at | LLP DAG | Mm.389704 | PREDICTED: Mus musculus | #N/A | 1E−26 |
| (SEQ ID NO: 2151) | | | similar to 40S ribosomal protein | | |
| | | | S2 (LOC623466), mRNA | | |
| WAN013HU8_at | LLP DAG | METAP1 | Methionyl aminopeptidase 1 | Hs.480364 | 1E−175 |
| (SEQ ID NO: 2152) | | | | | |
| WAN013HVQ_f_at | LLP DAG | H3F3B | H3 histone, family 3B (H3.3B) | Hs.180877 | 1E−130 |
| (SEQ ID NO: 2153) | | | | | |
| WAN013HW1_at | LLP DAG | Eef1d | Eukaryotic translation elongation | Hs.333388 | 1E−115 |
| (SEQ ID NO: 1989) | | | factor 1 delta (guanine | | |
| | | | nucleotide exchange protein) | | |
| WAN013HWP_x_at | LLP DAG | NA | Cluster includes WAN008CUN | #N/A | 0 |
| (SEQ ID NO: 1707) | | | 10602C-E01 | | |
| WAN013HX0_at | LLP DAG | NA | Cluster includes WAN008CXR | #N/A | 2E−07 |
| (SEQ ID NO: 2154) | | | 10603C-A08 | | |
| WAN013HX2_x_at | LLP DAG | NA | Cluster includes WAN008CWY | #N/A | 0 |
| (SEQ ID NO: 2155) | | | 10603C-D08 | | |
| WAN013HXU_at | LLP DAG | PSMA1 | Proteasome (prosome, | Hs.102798 | 0 |
| (SEQ ID NO: 2156) | | | macropain) subunit, alpha type, 1 | | |
| WAN013I3S_at | LLP DAG | Rpl19 | Ribosomal protein L19 | Hs.381061 | 0 |
| (SEQ ID NO: 2157) | | | | | |
| WAN013I40_at | LLP DAG | Mm.309697 | PREDICTED: Mus musculus | #N/A | 1E−139 |
| (SEQ ID NO: 2158) | | | similar to ribosomal protein S14, | | |
| | | | transcript variant 2 | | |
| | | | (LOC545121), mRNA | | |
| WAN013I98_at | LLP DAG | TST | Thiosulfate sulfurtransferase | Hs.474783 | 0 |
| (SEQ ID NO: 1818) | | | (rhodanese) | | |
| X98066_at | LLP DAG | TSN | Translin | Hs.75066 | 1E−87 |
| (SEQ ID NO: 2159) | | | | | |
| AF004814_at | LLP DCU | UBE2I | Ubiquitin-conjugating enzyme | Hs.302903 | 1E−157 |
| (SEQ ID NO: 2160) | | | E2I (UBC9 homolog, yeast) | | |
| WAN008939_at | LLP DCU | NA | WAN008939 10599B-G04 | #N/A | 5E−28 |
| (SEQ ID NO: 2161) | | | | | |
| WAN008DZC_x_at | LLP DCU | NA | WAN008DZC 11229B-E04 | #N/A | 0 |
| (SEQ ID NO: 2162) | | | | | |
| WAN013HVW_at | LLP DCU | Scd1 | Stearoyl-Coenzyme A | #N/A | 0 |
| (SEQ ID NO: 1926) | | | desaturase 1 | | |
| WAN013HWY_at | LLP DCU | CCDC80 | Coiled-coil domain containing 80 | Hs.477128 | 6E−92 |
| (SEQ ID NO: 1710) | | | | | |
| WAN013HZQ_at | LLP DCU | NA | Cluster includes WAN008DXN | #N/A | 0 |
| (SEQ ID NO: 2163) | | | 11229A-B12 | | |
| Y11149_at | LLP DCU | TEF | Thyrotrophic embryonic factor | Hs.181159 | 1E−147 |
| (SEQ ID NO: 2164) | | | | | |
| WAN0088J1_at | SHCV | Mrpl16 | Mitochondrial ribosomal protein | Hs.530734 | 1E−53 |
| (SEQ ID NO: 2165) | DAG | | L16 | | |
| WAN008BOI_at | SHCV | NA | WAN008BOI 11233D-B12 | #N/A | 0 |
| (SEQ ID NO: 2166) | DAG | | | | |
| WAN008BR0_at | SHCV | GOT2 | Glutamic-oxaloacetic | Hs.599470 | 0.000001 |
| (SEQ ID NO: 1773) | DAG | | transaminase 2, mitochondrial | | |
| | | | (aspartate aminotransferase 2) | | |
| WAN008BR4_at | SHCV | NA | WAN008BR4 11231C-G09 | #N/A | 3E−29 |
| (SEQ ID NO: 2167) | DAG | | | | |
| WAN008CLK_at | SHCV | RAB6A | RAB6A, member RAS oncogene | Hs.12152 | 2E−55 |
| (SEQ ID NO: 1552) | DAG | | family | | |
| WAN008CPX_at | SHCV | 2010003J03Rik | RIKEN cDNA 2010003J03 gene | #N/A | 1E−152 |
| (SEQ ID NO: 2168) | DAG | | | | |
| WAN008D2Y_at | SHCV | ZA20D2 | Zinc finger, A20 domain | #N/A | 0 |
| (SEQ ID NO: 2169) | DAG | | containing 2 | | |
| WAN008DTC_at | SHCV | 2700085E05Rik | RIKEN cDNA 2700085E05 gene | #N/A | 2E−99 |
| (SEQ ID NO: 2170) | DAG | | | | |
| WAN008ECX_at | SHCV | Cd151 | CD151 antigen | #N/A | 9E−85 |
| (SEQ ID NO: 1759) | DAG | | | | |
| WAN008EGM_at | SHCV | SPFH1 | SPFH domain family, member 1 | Hs.150087 | 7E−21 |
| (SEQ ID NO: 2171) | DAG | | | | |
| WAN008EHM_at | SHCV | CLU | Clusterin | Hs.436657 | 1E−71 |
| (SEQ ID NO: 1709) | DAG | | | | |
| WAN013HUO_at | SHCV | SUCLG1 | Succinate-CoA ligase, GDP- | Hs.270428 | 1E−148 |
| (SEQ ID NO: 2172) | DAG | | forming, alpha subunit | | |
| WAN013HY0_at | SHCV | PRPF19 | PRP19/PSO4 pre-mRNA | Hs.502705 | 0 |
| (SEQ ID NO: 2173) | DAG | | processing factor 19 homolog (S. cerevisiae) | | |
| WAN013I3P_at | SHCV | CAMLG | Calcium modulating ligand | Hs.529846 | 1E−147 |
| (SEQ ID NO: 1653) | DAG | | | | |
| WAN013I6C_at | SHCV | SLC16A1 | Solute carrier family 16 | Hs.75231 | 2E−26 |
| (SEQ ID NO: 1655) | DAG | | (monocarboxylic acid | | |
| | | | transporters), member 1 | | |
| WAN013I9G_at | SHCV | SLC3A2 | Solute carrier family 3 (activators | Hs.502769 | 1E−105 |
| (SEQ ID NO: 1663) | DAG | | of dibasic and neutral amino acid | | |
| | | | transport), member 2 | | |
| WAN013I9M_x_at | SHCV | TUBB2B | Tubulin, beta 2B | Hs.300701 | 0 |
| (SEQ ID NO: 2174) | DAG | | | | |
| WAN013I9N_at | SHCV | TUBB2C | Tubulin, beta 2C | Hs.433615 | 1E−154 |
| (SEQ ID NO: 2175) | DAG | | | | |
| M26640_at | SHCV | CLU | Clusterin | Hs.436657 | 7E−92 |
| (SEQ ID NO: 2054) | DCU | | | | |
| WAN0088OE_at | SHCV | Crk | V-crk sarcoma virus CT10 | Hs.638121 | 1E−161 |
| (SEQ ID NO: 2176) | DCU | | oncogene homolog (avian) | | |
| WAN0088S8_at | SHCV | SLC29A1 | Solute carrier family 29 | Hs.25450 | 3E−35 |
| (SEQ ID NO: 1591) | DCU | | (nucleoside transporters), | | |
| | | | member 1 | | |
| WAN008CRQ_at | SHCV | NA | WAN008CRQ 10602B-H08 | #N/A | 0 |
| (SEQ ID NO: 2077) | DCU | | | | |
| WAN008CSC_at | SHCV | GARS | Glycyl-tRNA synthetase | Hs.404321 | 1E−176 |
| (SEQ ID NO: 2177) | DCU | | | | |
| WAN008CTA_at | SHCV | NOLC1 | Nucleolar and coiled-body | Hs.523238 | 1E−101 |
| (SEQ ID NO: 1957) | DCU | | phosphoprotein 1 | | |
| WAN008CZB_at | SHCV | D10Wsu52e | DNA segment, Chr 10, Wayne | #N/A | 0 |
| (SEQ ID NO: 2178) | DCU | | State University 52, expressed | | |
| WAN008D0D_x_at | SHCV | Cflar | CASP8 and FADD-like apoptosis | Hs.390736 | 0 |
| (SEQ ID NO: 2179) | DCU | | regulator | | |
| WAN008D0K_at | SHCV | AA408296 | Expressed sequence AA408296 | #N/A | 1E−110 |
| (SEQ ID NO: 1877) | DCU | | | | |
| WAN008D6B_at | SHCV | NA | WAN008D6B 11164A-D04 | #N/A | 0 |
| (SEQ ID NO: 2180) | DCU | | | | |
| WAN008DMI_at | SHCV | ACSL5 | Acyl-CoA synthetase long-chain | Hs.11638 | 1E−118 |
| (SEQ ID NO: 1610) | DCU | | family member 5 | | |
| WAN008DQM_at | SHCV | XPNPEP1 | X-prolyl aminopeptidase | Hs.390623 | 1E−147 |
| (SEQ ID NO: 2181) | DCU | | (aminopeptidase P) 1, soluble | | |
| WAN008DTZ_at | SHCV | AMOTL2 | Angiomotin like 2 | Hs.426312 | 7E−76 |
| (SEQ ID NO: 2182) | DCU | | | | |
| WAN008E21_at | SHCV | MYC | V-myc myelocytomatosis viral | Hs.202453 | 3E−77 |
| (SEQ ID NO: 2183) | DCU | | oncogene homolog (avian) | | |
| WAN008E6J_x_at | SHCV | NA | WAN008E6J 11230B-F06 | #N/A | 0 |
| (SEQ ID NO: 2184) | DCU | | | | |
| WAN008EAJ_at | SHCV | Eif3s10 | Eukaryotic translation initiation | #N/A | 1E−129 |
| (SEQ ID NO: 2185) | DCU | | factor 3, subunit 10 (theta) | | |
| WAN008EAK_at | SHCV | ZDHHC6 | Zinc finger, DHHC-type | Hs.196990 | 1E−149 |
| (SEQ ID NO: 2186) | DCU | | containing 6 | | |
| WAN008EFI_at | SHCV | PRSS15 | Protease, serine, 15 | Hs.350265 | 1E−167 |
| (SEQ ID NO: 2187) | DCU | | | | |
| WAN008EQ0_at | SHCV | NA | WAN008EQ0 11232D-G01 | #N/A | 1E−107 |
| (SEQ ID NO: 2188) | DCU | | | | |
| WAN008EV8_at | SHCV | MTHFD2 | Methylenetetrahydrofolate | Hs.469030 | 7E−50 |
| (SEQ ID NO: 2189) | DCU | | dehydrogenase (NADP+ | | |
| | | | dependent) 2, | | |
| | | | methenyltetrahydrofolate | | |
| | | | cyclohydrolase | | |
| WAN008EYJ_at | SHCV | PHLDB1 | Pleckstrin homology-like domain, | Hs.504062 | 1E−122 |
| (SEQ ID NO: 2190) | DCU | | family B, member 1 | | |
| WAN008F02_at | SHCV | TFPI | Tissue factor pathway inhibitor | Hs.516578 | 3E−14 |
| (SEQ ID NO: 2191) | DCU | | (lipoprotein-associated | | |
| | | | coagulation inhibitor) | | |
| WAN008F1L_at | SHCV | Mxi1 | Max interacting protein 1 | Hs.501023 | 1E−127 |
| (SEQ ID NO: 1782) | DCU | | | | |
| WAN008F30_f_at | SHCV | Mm.389704 | PREDICTED: Mus musculus | #N/A | 1E−26 |
| (SEQ ID NO: 2151) | DCU | | similar to 40S ribosomal protein | | |
| | | | S2 (LOC623466), mRNA | | |
| WAN013HUC_at | SHCV | SOD1 | Superoxide dismutase 1, soluble | Hs.443914 | 2E−73 |
| (SEQ ID NO: 2192) | DCU | | (amyotrophic lateral sclerosis 1 | | |
| | | | (adult)) | | |
| WAN013HUX_at | SHCV | Cdv3 | Carnitine deficiency-associated | | 2E−39 |
| (SEQ ID NO: 2193) | DCU | | gene expressed in ventricle 3 | | |
| WAN013HZ9_at | SHCV | CSPG6 | Chondroitin sulfate proteoglycan | #N/A | 0 |
| (SEQ ID NO: 2194) | DCU | | 6 (bamacan) | | |
| WAN013I3K_at | SHCV | IDH1 | Isocitrate dehydrogenase 1 | Hs.11223 | 0 |
| (SEQ ID NO: 1723) | DCU | | (NADP+), soluble | | |
| WAN013I6G_at | SHCV | NA | Cluster includes M12252 | #N/A | 0 |
| (SEQ ID NO: 1999) | DCU | | Chinese hamster alpha-tubulin I | | |
| | | | mRNA, complete cds. | | |
| WAN013I8T_at | SHCV | Tuba2 | Tubulin, alpha 2 | #N/A | 0 |
| (SEQ ID NO: 1999) | DCU | | | | |
| WAN013I9K_at | SHCV | Gstm1 | Glutathione S-transferase, mu 1 | Hs.301961 | 1E−87 |
| (SEQ ID NO: 2195) | DCU | | | | |
| AF056934_at | SHQP | APEX1 | APEX nuclease (multifunctional | Hs.73722 | 1E−122 |
| (SEQ ID NO: 2196) | DAG | | DNA repair enzyme) 1 | | |
| S74024_at | SHQP | XPA | Xeroderma pigmentosum, | Hs.591907 | 4E−54 |
| (SEQ ID NO: 1832) | DAG | | complementation group A | | |
| WAN008CJT_at | SHQP | SSR2 | Signal sequence receptor, beta | Hs.74564 | 1E−132 |
| (SEQ ID NO: 2197) | DAG | | (translocon-associated protein | | |
| | | | beta) | | |
| WAN008D1G_at | SHQP | Cul4b | Cullin 4B | Hs.102914 | 1E−172 |
| (SEQ ID NO: 2198) | DAG | | | | |
| WAN008EFO_at | SHQP | TM9SF2 | Transmembrane 9 superfamily | Hs.130413 | 1E−153 |
| (SEQ ID NO: 1825) | DAG | | member 2 | | |
| WAN008EQU_at | SHQP | Eif4a1 | Eukaryotic translation initiation | Hs.129673 | 1E−126 |
| (SEQ ID NO: 2199) | DAG | | factor 4A1 | | |
| WAN008ERA_at | SHQP | NA | WAN008ERA 11232D-B06 | #N/A | 0 |
| (SEQ ID NO: 2200) | DAG | | | | |
| WAN013HUI_at | SHQP | HIP2 | Huntingtin interacting protein 2 | Hs.50308 | 0 |
| (SEQ ID NO: 2045) | DAG | | | | |
| WAN013I9V_at | SHQP | Pgk1 | Phosphoglycerate kinase 1 | Hs.78771 | 0 |
| (SEQ ID NO: 2201) | DAG | | | | |
| WAN013IA0_at | SHQP | NA | Cluster includes J00060 Chinese | #N/A | 0 |
| (SEQ ID NO: 1495) | DAG | | hamster hprt mRNA, complete | | |
| | | | cds. | | |
| AF371373_at | SHQP | NA | AF371373 Cricetulus griseus | #N/A | 1E−113 |
| (SEQ ID NO: 2202) | DCU | | hypothetical protein A1-3 mRNA, | | |
| | | | complete cds. | | |
| WAN008CY8_at | SHQP | PRDM5 | PR domain containing 5 | Hs.132593 | 2E−42 |
| (SEQ ID NO: 2203) | DCU | | | | |
| WAN008DP1_x_at | SHQP | NA | WAN008DP1 11228A-B07 | #N/A | 0 |
| (SEQ ID NO: 2204) | DCU | | | | |
| WAN008DRO_at | SHQP | NA | WAN008DRO 11228B-A06 | #N/A | 0 |
| (SEQ ID NO: 2205) | DCU | | | | |
| WAN008EGO_f_at | SHQP | NA | WAN008EGO 11231B-C08 | #N/A | 0 |
| (SEQ ID NO: 2206) | DCU | | | | |
| WAN008EME_at | SHQP | NA | WAN008EME 11232B-F08 | #N/A | 0 |
| (SEQ ID NO: 2207) | DCU | | | | |
| WAN013I8N_at | SHQP | IMPDH2 | IMP (inosine monophosphate) | Hs.476231 | 0 |
| (SEQ ID NO: 1776) | DCU | | dehydrogenase 2 | | |
| Y12074_at | SHQP | SLC35A1 | Solute carrier family 35 (CMP- | Hs.423163 | 1E−171 |
| (SEQ ID NO: 2208) | DCU | | sialic acid transporter), member | | |
| | | | A1 | | |

| | | | Mouse | | | | | |
|---|---|---|---|---|---|---|---|---|
| Qualifier List | % ID | % QC | Unigene ID | eValue | % ID | % QC | FC | Function |
| AB020230_at | 91.66667 | 7.5 | #N/A | 2E−17 | 92.537313 | 10.46875 | down | |
| (SEQ ID NO: 1665) | | | | | | | | |
| AF113614_at | 86.26506 | 80.89669 | Mm.87596 | 1E−167 | 92.325581 | 83.82066 | down | |
| (SEQ ID NO: 1668) | | | | | | | | |
| AF320819_at | 90.09901 | 17.11864 | Mm.726 | 4E−92 | 89.144737 | 51.52542 | down | |
| (SEQ ID NO: 1669) | | | | | | | | |
| M76730_at | 91.35802 | 30.97514 | Mm.7281 | 1E−110 | 96.442688 | 48.37476 | down | |
| (SEQ ID NO: 1671) | | | | | | | | |
| U62588_x_at | 93 | 53.19149 | Mm.2580 | 7E−48 | 90.849673 | 81.38298 | down | |
| (SEQ ID NO: 1586) | | | | | | | | |
| WAN0088J9_x_at | 87.6494 | 20.29103 | Mm.4189 | 4.00E−60 | 89.423077 | 16.81487 | 1.78 | |
| (SEQ ID NO: 1588) | | | | | | | | |
| WAN008BRK_at | 93.61702 | 71.75573 | Mm.142729 | 0 | 95.339806 | 98.28244 | down | |
| (SEQ ID NO: 1594) | | | | | | | | |
| WAN008BSG_x_at | 89.92248 | 36.23596 | Mm.28765 | 5E−44 | 91.78744 | 58.14607 | up | |
| (SEQ ID NO: 1595) | | | | | | | | |
| WAN008CHP_x_at | | | #N/A | | | | up | |
| (SEQ ID NO: 1596) | | | | | | | | |
| WAN008D3Z_at | 88.8668 | 100 | Mm.62886 | 1E−150 | 90.854871 | 100 | down | |
| (SEQ ID NO: 1602) | | | | | | | | |
| WAN008DFT_at | 83.21678 | 26.33517 | Mm.181473 | 9E−53 | 87.124464 | 42.90976 | 1.78 | |
| (SEQ ID NO: 1605) | | | | | | | | |
| WAN008DI7_at | | | Mm.28865 | 2E−23 | 86.956522 | 68.45238 | down | |
| (SEQ ID NO: 1607) | | | | | | | | |
| WAN008DIA_at | 90.55441 | 97.4 | Mm.311063 | 0 | 95.4 | 100 | up | |
| (SEQ ID NO: 1608) | | | | | | | | |
| WAN008DMJ_at | 89.57169 | 100 | Mm.336898 | 0 | 92.682927 | 99.25512 | up | |
| (SEQ ID NO: 1611) | | | | | | | | |
| WAN008DS9_at | 89.01869 | 90.87049 | Mm.276826 | 1E−132 | 92.982456 | 84.71338 | up | |
| (SEQ ID NO: 1613) | | | | | | | | |
| WAN008E06_at | 85.47215 | 76.34011 | Mm.35467 | 0 | 91.369606 | 98.52126 | down | |
| (SEQ ID NO: 1616) | | | | | | | | |
| WAN008EKK_at | 91.66667 | 98.63014 | Mm.87277 | 2E−89 | 90.181818 | 94.17808 | −1.58 | |
| (SEQ ID NO: 1625) | | | | | | | | |
| WAN008END_at | 83.33333 | 74.84407 | Mm.276063 | 0 | 92.291667 | 99.7921 | | |
| (SEQ ID NO: 1628) | | | | | | | | |
| WAN008EQM_at | | | #N/A | | | | −2.21 | |
| (SEQ ID NO: 1630) | | | | | | | | |
| WAN008ERO_at | 89.11565 | 28.21497 | Mm.33721 | 3E−40 | 90.510949 | 26.29559 | up | |
| (SEQ ID NO: 1633) | | | | | | | | |
| WAN008EY0_at | 90.21113 | 99.04943 | Mm.58660 | 1E−163 | 88.78327 | 100 | up | |
| (SEQ ID NO: 1636) | | | | | | | | |
| WAN013HVJ_at | 83.66834 | 71.58273 | #N/A | 1E−118 | 87.112172 | 75.35971 | down | |
| (SEQ ID NO: 1638) | | | | | | | | |
| WAN013HZK_at | | | #N/A | 7E−10 | 88.372093 | 17.58691 | 4 | |
| (SEQ ID NO: 1643) | | | | | | | | |
| WAN013HZP_at | 97.74648 | 72.44898 | Mm.185453 | 0 | 99.71831 | 72.44898 | 1.2 | |
| (SEQ ID NO: 1644) | | | | | | | | |
| WAN013I01_at | 89.56044 | 52 | Mm.30251 | 2E−35 | 84.699454 | 52.28571 | down | |
| (SEQ ID NO: 1645) | | | | | | | | |
| WAN013I15_at | 85.81952 | 100 | Mm.292637 | 0 | 88.39779 | 100 | 1.55 | |
| (SEQ ID NO: 1647) | | | | | | | | |
| WAN013I2F_at | 88.76404 | 18.05274 | Mm.24096 | 9E−93 | 85.714286 | 92.29209 | up | |
| (SEQ ID NO: 1649) | | | | | | | | |
| WAN013I2K_at | 91.07807 | 100 | Mm.130982 | 7E−86 | 89.962825 | 100 | down | |
| (SEQ ID NO: 1650) | | | | | | | | |
| WAN013I6C_at | 84.47205 | 12.69716 | Mm.9086 | 1E−110 | 87.239583 | 30.28391 | up | |
| (SEQ ID NO: 1655) | | | | | | | | |
| WAN013I6E_x_at | 81.99052 | 85.88874 | #N/A | 0 | 87.576687 | 88.46676 | down | |
| (SEQ ID NO: 1656) | | | | | | | | |
| WAN013I8B_at | 91.53713 | 99.31389 | Mm.30085 | 0 | 91.709845 | 99.31389 | down | |
| (SEQ ID NO: 1659) | | | | | | | | |
| WAN013I8V_at | 90.05848 | 67.05882 | Mm.154378 | 1E−137 | 93.274854 | 67.05882 | up | |
| (SEQ ID NO: 1660) | | | | | | | | |
| WAN013I9G_at | 84.85437 | 38.06356 | Mm.4114 | 0 | 88.596491 | 58.98004 | up | |
| (SEQ ID NO: 1663) | | | | | | | | |
| WAN0088PR_at | 87.5 | 11.42857 | Mm.268475 | 3E−34 | 90.243902 | 21.96429 | down | |
| (SEQ ID NO: 1589) | | | | | | | | |
| WAN0088Q6_at | 93.71585 | 63.43154 | Mm.261676 | 0 | 95.13382 | 71.2305 | down | |
| (SEQ ID NO: 1590) | | | | | | | | |
| WAN0088S8_at | 81.35593 | 76.12903 | Mm.29744 | 6E−97 | 86.097561 | 88.17204 | up | |
| (SEQ ID NO: 1591) | | | | | | | | |
| WAN0088T2_at | 88.53974 | 97.83002 | Mm.641 | 0 | 91.713748 | 96.0217 | up | |
| (SEQ ID NO: 1592) | | | | | | | | |
| WAN008CM7_x_at | 82.02247 | 25.35613 | Mm.354426 | 6E−13 | 84.466019 | 29.34473 | up | |
| (SEQ ID NO: 1597) | | | | | | | | |
| WAN008DGZ_at | 84.23423 | 79.42755 | Mm.269029 | 1E−139 | 89.861751 | 77.63864 | up | |
| (SEQ ID NO: 1606) | | | | | | | | |
| WAN008DJ8_f_at | 87.80488 | 24.84848 | Mm.331 | 2E−25 | 88.617886 | 24.84848 | down | |
| (SEQ ID NO: 1609) | | | | | | | | |
| WAN008DQE_at | 94.85294 | 100 | #N/A | 0 | 95.588235 | 100 | down | |
| (SEQ ID NO: 1612) | | | | | | | | |
| WAN008E2Q_at | 93.69565 | 100 | Mm.325827 | 0 | 95.869565 | 100 | up | |
| (SEQ ID NO: 1618) | | | | | | | | |
| WAN008EJY_at | | | #N/A | | | | up | |
| (SEQ ID NO: 1624) | | | | | | | | |
| WAN008ERB_at | 96.76113 | 99.39638 | Mm.274146 | 0 | 97.585513 | 100 | up | |
| (SEQ ID NO: 1631) | | | | | | | | |
| WAN013HVL_at | 89.38053 | 97.8355 | Mm.344831 | 1E−160 | 89.230769 | 98.48485 | up | |
| (SEQ ID NO: 1639) | | | | | | | | |
| WAN013HZ3_at | 91.83673 | 9.979633 | Mm.67949 | 4E−29 | 82.683983 | 47.04684 | down | |
| (SEQ ID NO: 1642) | | | | | | | | |
| WAN013I2L_at | 100 | 2.252252 | Mm.27943 | 1.00E−07 | 92 | 3.753754 | up | |
| (SEQ ID NO: 1651) | | | | | | | | |
| WAN013I3P_at | 86.70213 | 99.29577 | #N/A | 1E−172 | 88.6121 | 98.94366 | up | |
| (SEQ ID NO: 1653) | | | | | | | | |
| WAN013I61_at | | | Mm.2740 | 5E−30 | 88.28125 | 23.14647 | down | |
| (SEQ ID NO: 1654) | | | | | | | | |
| WAN013I6P_x_at | 87.71044 | 28.59206 | Mm.146649 | 0 | 89.771102 | 33.64621 | down | |
| (SEQ ID NO: 1658) | | | | | | | | |
| Y00365_at | 93.97993 | 23.39593 | Mm.313345 | 1E−145 | 88.84058 | 53.99061 | up | |
| (SEQ ID NO: 1674) | | | | | | | | |
| AF081143_at | 90.7173 | 98.34025 | Mm.324762 | 4E−93 | 92.946058 | 100 | 1.28 | |
| (SEQ ID NO: 1585) | | | | | | | | |
| WAN0088PT_at | 92.29167 | 99.58506 | Mm.157105 | 0 | 94.560669 | 99.17012 | down | protein |
| (SEQ ID NO: 1556) | | | | | | | | degradation |
| WAN0088XH_at | 87.41722 | 68.48073 | Mm.29151 | 1E−144 | 91.463415 | 92.97052 | −1.3 | UPR and ERAD |
| (SEQ ID NO: 1557) | | | | | | | | |
| WAN008CM1_x_at | 0 | 0 | 0 | #N/A | 0 | 0 | 0 | |
| (SEQ ID NO: 1559) | | | | | | | | |
| WAN008D6J_at | 94.80519 | 33.33333 | Mm.157190 | 1E−130 | 90.444444 | 97.4026 | −1.52 | transcriptional |
| (SEQ ID NO: 1563) | | | | | | | | cofactor |
| WAN008DSE_at | 86.89024 | 60.18349 | Mm.6379 | 1E−117 | 90.643275 | 62.75229 | −2.91 | glutamate/neutral |
| (SEQ ID NO: 1566) | | | | | | | | amino acid |
| | | | | | | | | transporter |
| WAN008EBJ_at | 87.61329 | 61.86916 | Mm.123714 | 1E−175 | 90.707071 | 92.52336 | −1.45 | cytoskeleton/ |
| (SEQ ID NO: 1569) | | | | | | | | actin binding |
| WAN008EFS_at | 86.77686 | 22.08029 | Mm.210155 | 2E−54 | 88.392857 | 40.87591 | −1.27 | pyrimidine |
| (SEQ ID NO: 1570) | | | | | | | | metabolism; |
| | | | | | | | | oxidative stress |
| WAN008EGV_at | 92.8972 | 94.85816 | Mm.8070 | 0 | 94.018692 | 94.85816 | −1.53 | regulates |
| (SEQ ID NO: 1571) | | | | | | | | GTPase activity |
| WAN008EMQ_at | 91.85336 | 100 | Mm.25548 | 0 | 96.334012 | 100 | −1.54 | nuclear transport |
| (SEQ ID NO: 1572) | | | | | | | | |
| WAN008ERL_at | 95.56962 | 87.05234 | Mm.379298 | 1E−162 | 95.867769 | 100 | −1.36 | mitochondrial |
| (SEQ ID NO: 1573) | | | | | | | | fatty acid beta- |
| | | | | | | | | oxidation |
| WAN008ETP_at | 86.94158 | 97.65101 | Mm.24576 | 8E−89 | 88.926174 | 100 | −1.3 | unknown |
| (SEQ ID NO: 1574) | | | | | | | | function |
| WAN008EX2_x_at | 90.29851 | 100 | Mm.168 | 6E−63 | 97.761194 | 100 | −1.45 | transcriptional |
| (SEQ ID NO: 1575) | | | | | | | | regulator; binds |
| | | | | | | | | HDAC's |
| WAN013HUM_at | 92.77108 | 59.00474 | Mm.132226 | 1E−132 | 89.285714 | 99.52607 | −1.46 | translocation; |
| (SEQ ID NO: 1576) | | | | | | | | endocytosis |
| WAN013HWG_at | 6E−34 | 83.47826 | 42.43542435 | #N/A | 0 | 0 | 0 | |
| (SEQ ID NO: 1577) | | | | | | | | |
| WAN013HYO_at | 90.52224 | 100 | Mm.276856 | 0 | 91.472868 | 99.80658 | 1.24 | |
| (SEQ ID NO: 1579) | | | | | | | | |
| WAN013I38_at | 90.52133 | 38.36364 | Mm.216135 | 0 | 92.989691 | 88.18182 | −1.3 | cell growth |
| (SEQ ID NO: 1583) | | | | | | | | |
| WAN008CWC_x_at | | | #N/A | | | | 1.365 | |
| (SEQ ID NO: 1560) | | | | | | | | |
| WAN008E8M_at | 88.19095 | 85.77586 | Mm.291463 | 1E−162 | 91.255605 | 96.12069 | −1.50 | |
| (SEQ ID NO: 1568) | | | | | | | | |
| U48852_at | 81.76944 | 55.13673 | Mm.292567 | 0 | 88.799355 | 91.7221 | up | |
| (SEQ ID NO: 1502) | | | | | | | | |
| WAN013HX9_at | 91.71717 | 93.75 | Mm.290251 | 0 | 94.507576 | 100 | 1.56 | |
| (SEQ ID NO: 2066) | | | | | | | | |
| WAN013HZ1_at | 89.57447 | 84.68468 | #N/A | 0 | 91.754757 | 85.22523 | 1.46 | |
| (SEQ ID NO: 2067) | | | | | | | | |
| WAN013166_f_at | 92.25589 | 57.6699 | Mm.268000 | 1E−131 | 91.84953 | 61.94175 | −1.97 | |
| (SEQ ID NO: 1494) | | | | | | | | |
| WAN013IAB_x_at | 82.44767 | 48.85917 | #N/A | 1E−133 | 81.320451 | 48.85917 | −1.45 | |
| (SEQ ID NO: 1496) | | | | | | | | |
| WAN0088OD_at | | | Mm.27769 | | | | down | |
| (SEQ ID NO: 2068) | | | | | | | | |
| WAN0088OT_at | | | #N/A | | | | down | |
| (SEQ ID NO: 1479) | | | | | | | | |
| WAN0088U4_at | | | | | | | down | |
| (SEQ ID NO: 2069) | | | | | | | | |
| WAN008DRM_at | | | Mm.9075 | | | | up | |
| (SEQ ID NO: 1503) | | | | | | | | |
| WAN008EEK_at | | | #N/A | | | | down | |
| (SEQ ID NO: 2070) | | | | | | | | |
| WAN008F2S_at | | | #N/A | | | | down | |
| SEQ ID NO: 1489 | | | | | | | | |
| AF115410_s_at | 86.09626 | 28.90263 | Mm.22001 | 2E−69 | 92.035398 | 34.93045 | up | |
| (SEQ ID NO: 2071) | | | | | | | | |
| AF157566_at | 86.81055 | 96.30485 | Mm.284853 | 1E−159 | 91.454965 | 100 | up | |
| (SEQ ID NO: 2072) | | | | | | | | |
| WAN0088SH_at | 91.15789 | 97.53593 | Mm.6766 | 0 | 94.045175 | 100 | up | |
| (SEQ ID NO: 2073) | | | | | | | | |
| WAN0088Y2_at | 90.99265 | 100 | Mm.356653 | 0 | 93.566176 | 100 | down | |
| (SEQ ID NO: 1882) | | | | | | | | |
| WAN0088ZI_at | 93.70504 | 100 | Mm.273997 | 0 | 96.402878 | 100 | down | |
| (SEQ ID NO: 2074) | | | | | | | | |
| WAN0088ZJ_at | 88.57678 | 100 | Mm.4580 | 0 | 93.796992 | 99.62547 | up | |
| (SEQ ID NO: 2075) | | | | | | | | |
| WAN00896L_f_at | 0 | 0 | #N/A | 0 | 0 | 0 | up | |
| (SEQ ID NO: 2076) | | | | | | | | |
| WAN008BNG_at | 93.77778 | 45.91837 | Mm.31247 | 1E−85 | 92.888889 | 45.91837 | up | |
| (SEQ ID NO: 1780) | | | | | | | | |
| WAN008CI5_at | 89.21283 | 68.6 | Mm.289747 | 1E−142 | 93.586006 | 68.6 | down | |
| (SEQ ID NO: 1839) | | | | | | | | |
| WAN008CRQ_at | 0 | 0 | #N/A | 7E−38 | 87.755102 | 43.1338 | up | |
| (SEQ ID NO: 2077) | | | | | | | | |
| WAN008CRX_at | 93.85343 | 77.47253 | Mm.213991 | 0 | 95.774648 | 78.02198 | down | |
| (SEQ ID NO: 2078) | | | | | | | | |
| WAN008D4W_at | 86.29738 | 97.44318 | Mm.373627 | 1E−96 | 89.389068 | 88.35227 | up | |
| (SEQ ID NO: 2079) | | | | | | | | |
| WAN008DGZ_at | 84.23423 | 79.42755 | #N/A | 1E−143 | 89.861751 | 77.63864 | down | |
| (SEQ ID NO: 1606) | | | | | | | | |
| WAN008DJI_at | 91.52542 | 48.86128 | Mm.28693 | 1E−120 | 89.333333 | 77.63975 | down | |
| (SEQ ID NO: 2080) | | | | | | | | |
| WAN008DKS_at | 92.16867 | 68.73706 | Mm.2720 | 0 | 93.390192 | 97.10145 | up | |
| (SEQ ID NO: 1787) | | | | | | | | |
| WAN008DL6_at | 85.06024 | 81.69291 | #N/A | 0 | 0 | 0 | up | |
| (SEQ ID NO: 2081) | | | | | | | | |
| WAN008DWE_at | 85.85859 | 38.22394 | Mm.7271 | 3E−81 | 90.118577 | 97.6834 | up | |
| (SEQ ID NO: 2082) | | | | | | | | |
| WAN008E71_at | 93.46939 | 51.90678 | Mm.208286 | 1E−97 | 93.469388 | 51.90678 | down | |
| (SEQ ID NO: 2083) | | | | | | | | |
| WAN008E8M_at | 88.19095 | 85.77586 | Mm.291463 | 1E−160 | 91.03139 | 96.12069 | up | |
| (SEQ ID NO: 1568) | | | | | | | | |
| WAN008EDZ_at | 85.99222 | 99.2278 | Mm.18210 | 1E−153 | 88.223938 | 100 | down | |
| (SEQ ID NO: 2084) | | | | | | | | |
| WAN008EHM_at | 91.38756 | 36.60245 | Mm.200608 | 1E−91 | 90.740741 | 47.28546 | up | |
| (SEQ ID NO: 1709) | | | | | | | | |
| WAN008EIX_at | 0 | 0 | #N/A | 0 | 0 | 0 | up | |
| (SEQ ID NO: 2085) | | | | | | | | |
| WAN008EP8_at | 91.71975 | 41.97861 | Mm.310476 | 2E−93 | 88.055556 | 96.25668 | down | |
| (SEQ ID NO: 2086) | | | | | | | | |
| WAN008EXG_at | 88.66499 | 95.43269 | Mm.215288 | 1E−145 | 91.105769 | 100 | down | |
| (SEQ ID NO: 1901) | | | | | | | | |
| WAN013HV6_x_at | 88.23529 | 16.66667 | #N/A | 1E−17 | 86.419753 | 19.85294 | up | |
| (SEQ ID NO: 2087) | | | | | | | | |
| WAN013HV8_x_at | 87.96992 | 54.28571 | #N/A | 1E−08 | 86.153846 | 26.53061 | up | |
| (SEQ ID NO: 2088) | | | | | | | | |
| WAN013HW2_at | 91.59213 | 99.28952 | Mm.18472 | 0 | 92.895204 | 100 | down | |
| (SEQ ID NO: 2089) | | | | | | | | |
| WAN13HZ6_at | 88.69779 | 70.29361 | Mm.3444 | 1E−144 | 90.092166 | 74.95682 | down | |
| (SEQ ID NO: 2090) | | | | | | | | |
| WAN013HZJ_at | 93.22344 | 100 | Mm.3868 | 0 | 96.520147 | 100 | down | |
| (SEQ ID NO: 2091) | | | | | | | | |
| WAN013I0C_at | 91.60839 | 72.95918 | Mm.10702 | 0 | 92.030361 | 89.62585 | down | |
| (SEQ ID NO: 2092) | | | | | | | | |
| WAN013I3P_at | 86.70213 | 99.29577 | #N/A | 1E−172 | 88.6121 | 98.94366 | down | |
| (SEQ ID NO: 1653) | | | | | | | | |
| WAN013I43_at | 87.55869 | 91.22056 | Mm.329631 | 1E−148 | 90.322581 | 92.93362 | up | |
| (SEQ ID NO: 2093) | | | | | | | | |
| WAN013I8C_at | 88.02281 | 39.02077 | Mm.259 | 0 | 94.230769 | 57.8635 | up | |
| (SEQ ID NO: 2094) | | | | | | | | |
| WAN008DT7_at | 83.94366 | 60.89194 | Mm.378931 | 1E−102 | 87.272727 | 66.03774 | up | |
| (SEQ ID NO: 1486) | | | | | | | | |
| WAN008EKU_at | 89.5122 | 99.51456 | Mm.431979 | 0 | 0 | 0 | up | |
| (SEQ ID NO: 1741) | | | | | | | | |
| WAN013I4A_at | 85.54502 | 64.03642 | Mm.4512 | 0 | 91.493056 | 43.70258 | up | |
| (SEQ ID NO: 2095) | | | | | | | | |
| AF242536_at | 91.66667 | 99.31034 | Mm.30199 | 0 | 94.645941 | 99.82759 | down | |
| (SEQ ID NO: 2096) | | | | | | | | |
| AJ223076_at | 89.83051 | 4.39315 | #N/A | 8E−52 | 83.409091 | 32.76247 | down | |
| (SEQ ID NO: 2097) | | | | | | | | |
| WAN008CTZ_at | 90.28213 | 56.56028 | Mm.252080 | 1E−153 | 93.989071 | 64.89362 | down | |
| (SEQ ID NO: 2098) | | | | | | | | |
| WAN008D16_at | 90.9589 | 65.29517 | Mm.306663 | 1E−164 | 94.850949 | 66.01073 | down | |
| (SEQ ID NO: 2099) | | | | | | | | |
| WAN008DCP_at | 90.84507 | 100 | Mm.28140 | 0 | 95.422535 | 100 | down | |
| (SEQ ID NO: 2100) | | | | | | | | |
| WAN008DIE_at | 90.41096 | 55.40797 | Mm.212395 | 1E−102 | 90.031153 | 60.91082 | down | |
| (SEQ ID NO: 2101) | | | | | | | | |
| WAN008DKD_at | 90.41769 | 100 | Mm.268010 | 1E−156 | 92.137592 | 100 | down | |
| (SEQ ID NO: 2102) | | | | | | | | |
| WAN008DMP_at | 90.52863 | 94.19087 | Mm.142822 | 0 | 92.982456 | 94.60581 | down | |
| (SEQ ID NO: 1968) | | | | | | | | |
| WAN008DOG_at | 0 | 0 | #N/A | 0 | 0 | 0 | up | |
| (SEQ ID NO: 2103) | | | | | | | | |
| WAN008E2D_at | 86.92308 | 24.90421 | Mm.23656 | 9E−62 | 94.267516 | 30.07663 | up | |
| (SEQ ID NO: 2104) | | | | | | | | |
| WAN008ECD_at | 94.43299 | 99.58932 | Mm.275281 | 0 | 96.701031 | 99.58932 | down | |
| (SEQ ID NO: 1898) | | | | | | | | |
| WAN008EGD_at | 0 | 0 | #N/A | 3E−18 | 96.428571 | 10.44776 | up | |
| (SEQ ID NO: 2105) | | | | | | | | |
| WAN008ERJ_x_at | 0 | 0 | #N/A | 2E−13 | 86.746988 | 69.7479 | down | |
| (SEQ ID NO: 2106) | | | | | | | | |
| WAN008ET2_at | 89.87854 | 44.58484 | Mm.252316 | 1E−103 | 90.939597 | 53.79061 | down | |
| (SEQ ID NO: 2107) | | | | | | | | |
| WAN008EVI-rc_at | 90.83333 | 51.06383 | Mm.12926 | 2E−76 | 90.638298 | 100 | down | |
| (SEQ ID NO: 1902) | | | | | | | | |
| WAN008F32_at | 0 | 0 | #N/A | 0 | 0 | 0 | up | |
| (SEQ ID NO: 2108) | | | | | | | | |
| WAN013HVM_at | 0 | 0 | #N/A | 4E−32 | 87.272727 | 32.16374 | up | |
| (SEQ ID NO: 2109) | | | | | | | | |
| WAN013HVV_at | 88.36735 | 91.41791 | Mm.182912 | 0 | 93.632959 | 99.62687 | down | |
| (SEQ ID NO: 2110) | | | | | | | | |
| WAN013HWQ_x_at | 88.74172 | 84.83146 | Mm.267258 | 2E−51 | 91.176471 | 95.50562 | up | |
| (SEQ ID NO: 2111) | | | | | | | | |
| WAN013HY1_at | 87.13551 | 85.73529 | Mm.293233 | 0 | 92.380952 | 92.64706 | down | |
| (SEQ ID NO: 2112) | | | | | | | | |
| WAN013I0A_x_at | 83.39695 | 99.05482 | Mm.51049 | 1E−173 | 89.694656 | 99.05482 | down | |
| (SEQ ID NO: 2113) | | | | | | | | |
| WAN013I26_at | 89.26746 | 98.16054 | Mm.279195 | 0 | 94.067797 | 98.66221 | down | |
| (SEQ ID NO: 2114) | | | | | | | | |
| WAN013I30_at | 90.54545 | 100 | Mm.87773 | 0 | 93.272727 | 100 | down | |
| (SEQ ID NO: 1528) | | | | | | | | |
| WAN013I3A_at | 87.27273 | 58.82353 | #N/A | 3E−80 | 85.529716 | 68.98396 | down | |
| (SEQ ID NO: 2115) | | | | | | | | |
| WAN013I8P_at | 86.23853 | 93.32192 | Mm.486 | 0 | 91.202873 | 95.37671 | down | |
| (SEQ ID NO: 1728) | | | | | | | | |
| X53077_x_at | 0 | 0 | #N/A | 0 | 0 | 0 | down | |
| (SEQ ID NO: 2116) | | | | | | | | |
| AF121895_at | 93.50649 | 38.98734 | #N/A | 3E−63 | 91.666667 | 16.20253 | up | |
| (SEQ ID NO: 2117) | | | | | | | | |
| WAN008BNY_at | 92.30769 | 84.0796 | Mm.4467 | 1E−146 | 94.378698 | 84.0796 | up | |
| (SEQ ID NO: 1791) | | | | | | | | |
| WAN008CNN_at | 87.24584 | 97.30216 | Mm.241484 | 0 | 90.28777 | 100 | up | |
| (SEQ ID NO: 2118) | | | | | | | | |
| WAN008EOG_at | 0 | 0 | #N/A | 1E−17 | 89.74359 | 48.14815 | up | |
| (SEQ ID NO: 2119) | | | | | | | | |
| WAN013HYK_at | 89.28571 | 14.50777 | Mm.235346 | 2E−13 | 93.75 | 13.81693 | down | |
| (SEQ ID NO: 1716) | | | | | | | | |
| Y12837_at | 96.12756 | 100 | Mm.259021 | 0 | 97.722096 | 100 | up | |
| (SEQ ID NO: 2120) | | | | | | | | |
| AF004831_at | 88.88889 | 6.766917 | Mm.240336 | 5E−84 | 89.440994 | 24.21053 | down | |
| (SEQ ID NO: 1536) | | | | | | | | |
| AF072727_at | 87.41722 | 59.80198 | Mm.200499 | 1E−134 | 87.474747 | 98.0198 | up | |
| (SEQ ID NO: 2121) | | | | | | | | |
| AF081142_at | 90.21277 | 50 | Mm.297372 | 1E−114 | 93.571429 | 59.57447 | up | |
| (SEQ ID NO: 2122) | | | | | | | | |
| D86467_at | 86.19048 | 71.91781 | Mm.856 | 2E−40 | 86.740331 | 61.9863 | down | |
| (SEQ ID NO: 2123) | | | | | | | | |
| M12329_g_at | 92.34973 | 100 | #N/A | 0 | 96.994536 | 100 | up | |
| (SEQ ID NO: 1942) | | | | | | | | |
| M22350_at | 84.57711 | 79.44664 | #N/A | 3E−57 | 90.607735 | 71.5415 | up | |
| (SEQ ID NO: 2124) | | | | | | | | |
| U49841_at | 87.85047 | 8.478605 | Mm.20931 | 1E−111 | 93.081761 | 25.1981 | down | |
| (SEQ ID NO: 2125) | | | | | | | | |
| WAN0088K3_at | 0 | 0 | #N/A | 7E−07 | 86.25 | 15.74803 | down | |
| (SEQ ID NO: 2126) | | | | | | | | |
| WAN0088MM_at | 0 | 0 | #N/A | 9E−16 | 84.269663 | 32.42259 | down | |
| (SEQ ID NO: 2127) | | | | | | | | |
| WAN0088PU_at | 92.61538 | 62.74131 | Mm.34319 | 0 | 92.815534 | 99.42085 | up | |
| (SEQ ID NO: 2128) | | | | | | | | |
| WAN0088X9_at | 89.17379 | 66.73004 | Mm.275864 | 1E−161 | 92.156863 | 87.26236 | up | |
| (SEQ ID NO: 1553) | | | | | | | | |
| WAN0088Z7_at | 90.47619 | 100 | Mm.352239 | 0 | 93.121693 | 100 | up | |
| (SEQ ID NO: 2129) | | | | | | | | |
| WAN008900_at | 90.52823 | 99.81818 | Mm.259688 | 0 | 95.264117 | 99.81818 | down | |
| (SEQ ID NO: 2130) | | | | | | | | |
| WAN008CN4_at | 0 | 0 | #N/A | 1E−14 | 89.189189 | 14.01515 | down | |
| (SEQ ID NO: 2131) | | | | | | | | |
| WAN008CS2_at | 91.89189 | 96.73203 | Mm.288718 | 0 | 97.285068 | 96.2963 | down | |
| (SEQ ID NO: 1694) | | | | | | | | |
| WAN008CSN_at | 87.82961 | 99.79757 | #N/A | 0 | 92.712551 | 100 | down | |
| (SEQ ID NO: 2132) | | | | | | | | |
| WAN008D3X_at | 89.6875 | 59.81308 | Mm.277719 | 1E−161 | 94.010417 | 71.7757 | down | |
| (SEQ ID NO: 2133) | | | | | | | | |
| WAN008DBL_at | 85.76923 | 93.86282 | Mm.322294 | 1E−180 | 90.679612 | 92.96029 | up | |
| (SEQ ID NO: 1730) | | | | | | | | |
| WAN008DK1_at | 85.66879 | 64.87603 | Mm.335460 | 1E−110 | 91.082803 | 64.87603 | up | |
| (SEQ ID NO: 1829) | | | | | | | | |
| WAN008DLQ_at | 80.27613 | 99.80315 | Mm.41 | 1E−139 | 87.351779 | 99.6063 | up | |
| (SEQ ID NO: 2134) | | | | | | | | |
| WAN008DNO_at | 88.78719 | 80.18349 | Mm.33870 | 1E−160 | 90.212766 | 86.23853 | down | |
| (SEQ ID NO: 2135) | | | | | | | | |
| WAN008DRQ_x_at | 84.52381 | 100 | Mm.158231 | 1E−16 | 88.095238 | 100 | down | |
| (SEQ ID NO: 2136) | | | | | | | | |
| WAN008DXB_f_at | 0 | 0 | #N/A | 0 | 0 | 0 | up | |
| (SEQ ID NO: 2137) | | | | | | | | |
| WAN008E1P_at | 84.32304 | 75.99278 | Mm.274811 | 0 | 92.960289 | 100 | up | |
| (SEQ ID NO: 2138) | | | | | | | | |
| WAN008E8D_at | 93.12039 | 98.54722 | Mm.222328 | 0 | 97.783251 | 98.30508 | down | |
| (SEQ ID NO: 2139) | | | | | | | | |
| WAN008EEE_at | 96.33803 | 100 | Mm.289662 | 1E−160 | 98.309859 | 100 | down | |
| (SEQ ID NO: 2140) | | | | | | | | |
| WAN008EH6_at | 93.63636 | 97.86477 | Mm.2863 | 0 | 95.272727 | 97.86477 | down | |
| (SEQ ID NO: 2141) | | | | | | | | |
| WAN008EHM_at | 91.38756 | 38.60245 | Mm.200608 | 1E−91 | 90.740741 | 47.28546 | up | |
| (SEQ ID NO: 1709) | | | | | | | | |
| WAN008EK5- | 92.66055 | 21.84369 | #N/A | 7E−44 | 94.782609 | 23.04609 | down | |
| rc_x_at | | | | | | | | |
| (SEQ ID NO: 1979) | | | | | | | | |
| WAN008EKV_at | 90.8 | 51.12474 | Mm.30221 | 0 | 91.908714 | 98.56851 | down | |
| (SEQ ID NO: 2142) | | | | | | | | |
| WAN008EN3_at | 0 | 0 | Mm.29482 | 3E−78 | 90.163934 | 95.3125 | down | |
| (SEQ ID NO: 2142) | | | | | | | | |
| WAN008EOR_x_at | 0 | 0 | #N/A | 0 | 0 | 0 | down | |
| (SEQ ID NO: 2144) | | | | | | | | |
| WAN008EPF_at | 97.07792 | 98.71795 | Mm.317701 | 1E−152 | 97.077922 | 98.71795 | down | |
| (SEQ ID NO: 2145) | | | | | | | | |
| WAN008EPP_at | 0 | 0 | Mm.291799 | 3E−20 | 89.010989 | 29.93421 | down | |
| (SEQ ID NO: 2146) | | | | | | | | |
| WAN008ERQ_at | 90.43478 | 92 | Mm.335292 | 4E−93 | 93.248945 | 94.8 | down | |
| (SEQ ID NO: 2147) | | | | | | | | |
| WAN008ESB_at | 88.09524 | 17.17791 | #N/A | 2E−25 | 98.4375 | 13.08793 | down | |
| (SEQ ID NO: 2148) | | | | | | | | |
| WAN008ESS_at | 0 | 0 | #N/A | 4E−14 | 92.857143 | 17.99486 | down | |
| (SEQ ID NO: 2149) | | | | | | | | |
| WAN008ET4_at | 91.47122 | 99.78723 | Mm.29045 | 0 | 94.468085 | 100 | down | |
| (SEQ ID NO: 2150) | | | | | | | | |
| WAN008EWS_at | 93.45133 | 100 | Mm.22724 | 0 | 93.451327 | 100 | down | |
| (SEQ ID NO: 1688) | | | | | | | | |
| WAN008F30_f_at | 94.18605 | 43.65482 | #N/A | 2E−41 | 87.700535 | 94.92386 | up | |
| (SEQ ID NO: 2151) | | | | | | | | |
| WAN013HU8_at | 89.98035 | 99.22027 | Mm.26833 | 0 | 92.730845 | 99.22027 | down | |
| (SEQ ID NO: 2152) | | | | | | | | |
| WAN013HVQ_f_at | 90.93333 | 100 | Mm.18516 | 1E−166 | 94.4 | 100 | up | |
| (SEQ ID NO: 2153) | | | | | | | | |
| WAN013HW1_at | 84.05797 | 99.45946 | Mm.258927 | 0 | 91.240876 | 98.73874 | up | |
| (SEQ ID NO: 1989) | | | | | | | | |
| WAN013HWP_x_at | 0 | 0 | #N/A | 8E−76 | 92.822967 | 45.93407 | down | |
| (SEQ ID NO: 1707) | | | | | | | | |
| WAN013HX0_at | 90.90909 | 8.747515 | #N/A | 8E−15 | 84.313725 | 20.27833 | down | |
| (SEQ ID NO: 2154) | | | | | | | | |
| WAN013HX2_x_at | 0 | 0 | #N/A | 0 | 0 | 0 | up | |
| (SEQ ID NO: 2155) | | | | | | | | |
| WAN013HXU_at | 92.22011 | 100 | Mm.121265 | 0 | 94.847328 | 99.43074 | down | |
| (SEQ ID NO: 2156) | | | | | | | | |
| WAN013I3S_at | 88.95028 | 98.36957 | Mm.10247 | 0 | 92.753623 | 100 | up | |
| (SEQ ID NO: 2157) | | | | | | | | |
| WAN013I40_at | 90.72682 | 100 | #N/A | 1E−165 | 93.483709 | 100 | up | |
| (SEQ ID NO: 2158) | | | | | | | | |
| WAN013I98_at | 84.09332 | 87.23404 | Mm.15312 | 0 | 90.04329 | 85.47641 | up | |
| (SEQ ID NO: 1818) | | | | | | | | |
| X98066_at | 91.30435 | 47.02602 | Mm.426637 | 0 | 0 | 0 | down | |
| (SEQ ID NO: 2159) | | | | | | | | |
| AF004814_at | 89.15663 | 49.25816 | Mm.240044 | 0 | 93.655589 | 65.47972 | up | |
| (SEQ ID NO: 2160) | | | | | | | | |
| WAN008939_at | 90.26549 | 20.58288 | #N/A | 0 | 0 | 0 | up | |
| (SEQ ID NO: 2161) | | | | | | | | |
| WAN008DZC_x_at | 0 | 0 | #N/A | 0 | 0 | 0 | up | |
| (SEQ ID NO: 2162) | | | | | | | | |
| WAN013HVW_at | 0 | 0 | Mm.193096 | 8E−07 | 88.679245 | 9.330986 | down | |
| (SEQ ID NO: 1926) | | | | | | | | |
| WAN013HWY_at | 86.55914 | 76.07362 | Mm.181074 | 1E−171 | 90.57377 | 99.7955 | up | |
| (SEQ ID NO: 1710) | | | | | | | | |
| WAN013HZQ_at | 0 | 0 | #N/A | 0 | 0 | 0 | up | |
| (SEQ ID NO: 2163) | | | | | | | | |
| Y11149_at | 90.86538 | 71.84801 | Mm.270278 | 1E−173 | 93.509615 | 71.84801 | up | |
| (SEQ ID NO: 2164) | | | | | | | | |
| WAN0088J1_at | 90 | 34.1556 | Mm.203928 | 1E−103 | 94.023904 | 47.62808 | down | |
| (SEQ ID NO: 2165) | | | | | | | | |
| WAN008BOI_at | 0 | 0 | #N/A | 6E−25 | 85.096154 | 58.10056 | down | |
| (SEQ ID NO: 2166) | | | | | | | | |
| WAN008BR0_at | 83.95062 | 15.0838 | Mm.230169 | 6E−26 | 88.80597 | 24.95345 | down | |
| (SEQ ID NO: 1773) | | | | | | | | |
| WAN008BR4_at | 90.51724 | 24.26778 | #N/A | 2E−10 | 90.909091 | 11.50628 | down | |
| (SEQ ID NO: 2167) | | | | | | | | |
| WAN008CLK_at | 88.47737 | 48.21429 | #N/A | 1E−161 | 92.073171 | 97.61905 | down | |
| (SEQ ID NO: 1552) | | | | | | | | |
| WAN008CPX_at | 89.52991 | 94.54545 | Mm.299167 | 0 | 92.244898 | 98.9899 | down | |
| (SEQ ID NO: 2168) | | | | | | | | |
| WAN008D2Y_at | 94.58333 | 97.56098 | #N/A | 0 | 97.959184 | 99.5935 | down | |
| (SEQ ID NO: 2169) | | | | | | | | |
| WAN008DTC_at | 92.69231 | 98.48485 | Mm.249700 | 4E−99 | 92.664093 | 98.10606 | up | |
| (SEQ ID NO: 2170) | | | | | | | | |
| WAN008ECX_at | 86.36364 | 65.0647 | Mm.30246 | 1E−155 | 94.857143 | 64.69501 | down | |
| (SEQ ID NO: 1759) | | | | | | | | |
| WAN008EGM_at | 83.53659 | 29.76407 | Mm.279865 | 1E−122 | 86.912065 | 88.74773 | down | |
| (SEQ ID NO: 2171) | | | | | | | | |
| WAN008EHM_at | 91.38756 | 36.60245 | Mm.200608 | 1E−91 | 90.740741 | 47.28546 | down | |
| (SEQ ID NO: 1709) | | | | | | | | |
| WAN013HUO_at | 85.66553 | 98.98649 | Mm.29845 | 1E−159 | 86.713287 | 96.62162 | down | |
| (SEQ ID NO: 2172) | | | | | | | | |
| WAN013HY0_at | 89.25319 | 100 | Mm.358657 | 0 | 93.260474 | 100 | down | |
| (SEQ ID NO: 2173) | | | | | | | | |
| WAN013I3P_at | 86.70213 | 99.29577 | #N/A | 1E−172 | 88.6121 | 98.94366 | down | |
| (SEQ ID NO: 1653) | | | | | | | | |
| WAN013I6C_at | 84.47205 | 12.69716 | Mm.9086 | 1E−111 | 87.239583 | 30.28391 | down | |
| (SEQ ID NO: 1655) | | | | | | | | |
| WAN013I9G_at | 84.85437 | 38.06356 | Mm.4114 | 0 | 88.596491 | 58.98004 | down | |
| (SEQ ID NO: 1663) | | | | | | | | |
| WAN013I9M_x_at | 85.36804 | 80.20158 | #N/A | 0 | 88.778878 | 87.25702 | up | |
| (SEQ ID NO: 2174) | | | | | | | | |
| WAN013I9N_at | 91.07981 | 78.30882 | Mm.227260 | 0 | 95.194085 | 99.44853 | up | |
| (SEQ ID NO: 2175) | | | | | | | | |
| M26640_at | 82.96296 | 94.57093 | Mm.200608 | 0 | 91.843972 | 98.77408 | down | |
| (SEQ ID NO: 2054) | | | | | | | | |
| WAN0088OE_at | 94.35484 | 77.01863 | Mm.280125 | 0 | 97.043011 | 77.01863 | down | |
| (SEQ ID NO: 2176) | | | | | | | | |
| WAN0088S8_at | 81.35593 | 76.12903 | Mm.29744 | 5E−97 | 86.097561 | 88.17204 | down | |
| (SEQ ID NO: 1591) | | | | | | | | |
| WAN008CRQ_at | 0 | 0 | #N/A | 7E−38 | 87.755102 | 43.1338 | down | |
| (SEQ ID NO: 2077) | | | | | | | | |
| WAN008CSC_at | 89.14591 | 99.29329 | Mm.250004 | 0 | 92.932862 | 100 | down | |
| (SEQ ID NO: 2177) | | | | | | | | |
| WAN008CTA_at | 89.12387 | 59.63964 | Mm.402190 | 3E−28 | 89.908257 | 19.63964 | down | |
| (SEQ ID NO: 1957) | | | | | | | | |
| WAN008CZB_at | 93.05556 | 100 | Mm.9257 | 0 | 94.715447 | 97.61905 | down | |
| (SEQ ID NO: 2178) | | | | | | | | |
| WAN008D0D_x_at | 0 | 0 | Mm.11778 | 3E−25 | 85.276074 | 28.69718 | down | |
| (SEQ ID NO: 2179) | | | | | | | | |
| WAN008D0K_at | 86.77494 | 96.85393 | Mm.173758 | 1E−143 | 89.864865 | 99.77528 | down | |
| (SEQ ID NO: 1877) | | | | | | | | |
| WAN008D6B_at | 0 | 0 | #N/A | 0 | 0 | 0 | down | |
| (SEQ ID NO: 2180) | | | | | | | | |
| WAN008DMI_at | 85 | 96.60107 | Mm.292056 | 0 | 89.94614 | 99.64222 | down | |
| (SEQ ID NO: 1610) | | | | | | | | |
| WAN008DQM_at | 88.11881 | 99.60552 | Mm.99776 | 0 | 92.504931 | 100 | down | |
| (SEQ ID NO: 2181) | | | | | | | | |
| WAN008DTZ_at | 87.67606 | 63.39286 | Mm.21145 | 1E−106 | 88.732394 | 79.24107 | up | |
| (SEQ ID NO: 2182) | | | | | | | | |
| WAN008E21_at | 93.10345 | 66.33987 | Mm.2444 | 2E−76 | 92.018779 | 69.60784 | down | |
| (SEQ ID NO: 2183) | | | | | | | | |
| WAN008E6J_x_at | 0 | 0 | #N/A | 2E−06 | 89.361702 | 40.86957 | down | |
| (SEQ ID NO: 2184) | | | | | | | | |
| WAN008EAJ_at | 86.45038 | 98.49624 | Mm.2238 | 0 | 94.274809 | 98.49624 | down | |
| (SEQ ID NO: 2185) | | | | | | | | |
| WAN008EAK_at | 90.09009 | 91.92547 | Mm.386789 | 1E−158 | 90.950226 | 91.51139 | down | |
| (SEQ ID NO: 2186) | | | | | | | | |
| WAN008EFI_at | 88.27709 | 98.2548 | Mm.329136 | 0 | 91.448517 | 100 | down | |
| (SEQ ID NO: 2187) | | | | | | | | |
| WAN008EQ0_at | 89.44282 | 67.79324 | #N/A | 1E−143 | 93.641618 | 68.78728 | down | |
| (SEQ ID NO: 2188) | | | | | | | | |
| WAN008EV8_at | 88.35979 | 68.23105 | Mm.443 | 4E−84 | 91.056911 | 88.80866 | down | |
| (SEQ ID NO: 2189) | | | | | | | | |
| WAN008EYJ_at | 93.62416 | 74.31421 | Mm.28639 | 1E−134 | 95.049505 | 75.5611 | up | |
| (SEQ ID NO: 2190) | | | | | | | | |
| WAN008F02_at | 83.33333 | 21.79931 | Mm.124316 | 3E−83 | 85.522788 | 64.53287 | up | |
| (SEQ ID NO: 2191) | | | | | | | | |
| WAN008F1L_at | 90.23355 | 84.86486 | Mm.2154 | 1E−131 | 88.447653 | 99.81982 | down | |
| (SEQ ID NO: 1782) | | | | | | | | |
| WAN008F30_f_at | 94.18605 | 43.65482 | #N/A | 2E−41 | 87.700535 | 94.92386 | down | |
| (SEQ ID NO: 2151) | | | | | | | | |
| WAN013HUC_at | 85.71429 | 57.21739 | Mm.276325 | 1E−126 | 91.036415 | 62.08696 | up | |
| (SEQ ID NO: 2192) | | | | | | | | |
| WAN013HUX_at | 90.90909 | 34.714 | Mm.261025 | 1E−156 | 85.915493 | 98.02761 | down | |
| (SEQ ID NO: 2193) | | | | | | | | |
| WAN013HZ9_at | 90.15544 | 98.80546 | #N/A | 0 | 92.943201 | 99.14676 | down | |
| (SEQ ID NO: 2194) | | | | | | | | |
| WAN013I3K_at | 91.04478 | 99.44341 | Mm.9925 | 0 | 93.470149 | 99.44341 | up | |
| (SEQ ID NO: 1723) | | | | | | | | |
| WAN013I6G_at | 0 | 0 | #N/A | 0 | 0 | 0 | up | |
| (SEQ ID NO: 1999) | | | | | | | | |
| WAN013I8T_at | 90.74941 | 79.66418 | Mm.392113 | 0 | 94.362018 | 94.3097 | up | |
| (SEQ ID NO: 1999) | | | | | | | | |
| WAN013I9K_at | 84.64912 | 79.16667 | Mm.37199 | 1E−175 | 90.558767 | 90.10417 | up | |
| (SEQ ID NO: 2195) | | | | | | | | |
| AF056934_at | 87.04104 | 88.19048 | Mm.203 | 1E−159 | 90.17094 | 89.14286 | up | |
| (SEQ ID NO: 2196) | | | | | | | | |
| S74024_at | 87.73585 | 96.36364 | Mm.247036 | 6E−64 | 89.090909 | 100 | up | |
| (SEQ ID NO: 1832) | | | | | | | | |
| WAN008CJT_at | 89.39759 | 78.44991 | Mm.7091 | 1E−179 | 90.766208 | 96.21928 | down | |
| (SEQ ID NO: 2197) | | | | | | | | |
| WAN008D1G_at | 89.72868 | 99.6139 | Mm.327675 | 0 | 90.891473 | 99.6139 | down | |
| (SEQ ID NO: 2198) | | | | | | | | |
| WAN008EFO_at | 90.94203 | 100 | Mm.275191 | 0 | 95.471014 | 100 | down | |
| (SEQ ID NO: 1825) | | | | | | | | |
| WAN008EQU_at | 92.40122 | 97.62611 | Mm.279821 | 1E−148 | 94.658754 | 100 | down | |
| (SEQ ID NO: 2199) | | | | | | | | |
| WAN008ERA_at | 0 | 0 | #N/A | 3E−24 | 89.130435 | 30.9417 | down | |
| (SEQ ID NO: 2200) | | | | | | | | |
| WAN013HUI_at | 96.53061 | 94.77756 | Mm.319512 | 0 | 97.270955 | 99.22631 | up | |
| (SEQ ID NO: 2045) | | | | | | | | |
| WAN013I9V_at | 92.71028 | 42.39303 | Mm.316355 | 0 | 94.827586 | 45.9588 | down | |
| (SEQ ID NO: 2201) | | | | | | | | |
| WAN013IA0_at | 0 | 0 | #N/A | 2E−12 | 84.615385 | 31.70732 | down | |
| (SEQ ID NO: 1495) | | | | | | | | |
| AF371373_at | 84.87713 | 39.36012 | #N/A | 0 | 87.015177 | 88.24405 | down | |
| (SEQ ID NO: 2202) | | | | | | | | |
| WAN008CY8_at | 82.01058 | 77.77778 | Mm.263355 | 5E−91 | 85.523385 | 92.38683 | up | |
| (SEQ ID NO: 2203) | | | | | | | | |
| WAN008DP1_x_at | 0 | 0 | #N/A | 0 | 0 | 0 | down | |
| (SEQ ID NO: 2204) | | | | | | | | |
| WAN008DRO_at | 0 | 0 | #N/A | 4E−27 | 86.956522 | 27.00587 | up | |
| (SEQ ID NO: 2205) | | | | | | | | |
| WAN008EGO_f_at | 0 | 0 | #N/A | 0 | 0 | 0 | up | |
| (SEQ ID NO: 2206) | | | | | | | | |
| WAN008EME_at | 0 | 0 | #N/A | 0 | 0 | 0 | down | |
| (SEQ ID NO: 2207) | | | | | | | | |
| WAN013I8N_at | 90.28974 | 95.36968 | Mm.6065 | 0 | 93.183579 | 96.41524 | up | |
| (SEQ ID NO: 1776) | | | | | | | | |
| Y12074_at | 90.07634 | 39.30983 | Mm.281885 | 0 | 91.732909 | 47.1868 | down | |
| (SEQ ID NO: 2208) | | | | | | | | |

TABLE 26
High Priority Gene list 3

| | | | | Human | | |
|---|---|---|---|---|---|---|
| Qualifier List | Category | Symbol | Title | Unigene ID | eValue | % ID |
| L00176_at | HMQP DAG | HMGCR | 3-hydroxy-3-methylglutaryl- | Hs.11899 | 6E−54 | 87.98077 |
| (SEQ ID | | | Coenzyme A reductase | | | |
| NO: 1500) | | | | | | |
| WAN0088ZP_at | HMQP DAG | PAWR | PRKC, apoptosis, WT1, regulator | Hs.406074 | 4E−10 | 91.52542 |
| (SEQ ID | | | | | | |
| NO: 1482) | | | | | | |
| WAN008CJ1_at | HMQP DAG | ERP70 | Protein disulfide isomerase- | Hs.93659 | 1E−120 | 89.62766 |
| (SEQ ID | | | associated 4 | | | |
| NO: 1485) | | | | | | |
| WAN008EA0_at | HMQP DAG | VCP | Valosin-containing protein | Hs.529782 | 0 | 90.72727 |
| (SEQ ID | | | | | | |
| NO: 1487) | | | | | | |
| D45419_at | HMQP DCU | Hcfc1 | Host cell factor C1 | Hs.83634 | 1E−22 | 84.86486 |
| (SEQ ID | | | | | | |
| NO: 1498) | | | | | | |
| WAN0088NU_at | HMQP DCU | LMNA | Lamin A/C | Hs.491359 | 3E−23 | 85.71429 |
| (SEQ ID | | | | | | |
| NO: 1478) | | | | | | |
| WAN008F1I_at | | SHOC2 | Soc-2 suppressor of clear | Hs.104315 | 2E−18 | 85.05747 |
| (SEQ ID | | | homolog (C. elegans) | | | |
| NO: 1488) | | | | | | |
| WAN013I1P_at | | HNRPA2B1 | Heterogeneous nuclear | Hs.487774 | 0 | 97.22222 |
| (SEQ ID | | | ribonucleoprotein A2/B1 | | | |
| NO: 1492) | | | | | | |
| WAN008DT7_at | | GSTO1 | Glutathione S-transferase | Hs.190028 | 5E−65 | 83.94366 |
| (SEQ ID | | | omega 1 | | | |
| NO: 1486) | | | | | | |

| | | Mouse | | | | | |
|---|---|---|---|---|---|---|---|
| Qualifier List | % QC | Unigene ID | eValue | % ID | % QC | FC | Function |
| L00176_at | 57.93872 | Mm.316652 | 4E−82 | 94.47236 | 55.43175 | up | |
| (SEQ ID | | | | | | | |
| NO: 1500) | | | | | | | |
| WAN0088ZP_at | 11.11111 | Mm.336104 | 9E−53 | 91.62562 | 38.22976 | up | |
| (SEQ ID | | | | | | | |
| NO: 1482) | | | | | | | |
| WAN008CJ1_at | 81.91721 | Mm.2442 | 1E−170 | 94.57364 | 84.31373 | up | |
| (SEQ ID | | | | | | | |
| NO: 1485) | | | | | | | |
| WAN008EA0_at | 99.63768 | Mm.379457 | 0 | 95.47101 | 100 | up | |
| (SEQ ID | | | | | | | |
| NO: 1487) | | | | | | | |
| D45419_at | 32.74336 | Mm.248353 | 1E−123 | 85.99291 | 99.82301 | down | transcription |
| (SEQ ID | | | | | | | factor |
| NO: 1498) | | | | | | | |
| WAN0088NU_at | 23.05026 | Mm.243014 | 0 | 92.04947 | 98.09359 | down | |
| (SEQ ID | | | | | | | |
| NO: 1478) | | | | | | | |
| WAN008F1I_at | 31.07143 | Mm.228669 | 1E−36 | 90.37037 | 24.10714 | −2.62 | |
| (SEQ ID | | | | | | | |
| NO: 1488) | | | | | | | |
| WAN013I1P_at | 90.94737 | Mm.155896 | 0 | 96.52778 | 90.94737 | −3.49 | pre-mRNA |
| (SEQ ID | | | | | | | processing |
| NO: 1492) | | | | | | | |
| WAN008DT7_at | 60.89194 | Mm.378931 | 1E−102 | 87.27273 | 66.03774 | −2.11 | redox |
| (SEQ ID | | | | | | | homeostasis; |
| NO: 1486) | | | | | | | stress |
| | | | | | | | response |

TABLE 27
HCGR 2 + 3 Overlap 1.2F UP (10)

| | | | Human | | |
|---|---|---|---|---|---|
| Qualifier List | Symbol | Title | Unigene ID | eValue | % ID |
| WAN013I0S_at | 4930548G07Rik | RIKEN cDNA 4930548G07 | #N/A | 4E−67 | 87.21804511 |
| (SEQ ID NO: 2209) | | gene | | | |
| WAN0088WF_at | DHX36 | DEAH (Asp-Glu-Ala-His) box | Hs.446270 | 2E−97 | 92.0754717 |
| (SEQ ID NO: 1874) | | polypeptide 36 | | | |
| WAN008EVU_x_at | HNRPK | Heterogeneous nuclear | Hs.522257 | 1E−129 | 92.98780488 |
| (SEQ ID NO: 1887) | | ribonucleoprotein K | | | |
| X51747_at | HSPB1 | Heat shock 27 kDa protein 1 | Hs.520973 | 1E−101 | 87.36842105 |
| (SEQ ID NO: 1587) | | | | | |
| WAN008D3S_x_at | IDE | Insulin-degrading enzyme | Hs.500546 | 0.0000001 | 89.65517241 |
| (SEQ ID NO: 2210) | | | | | |
| U62587_at | MGAT5 | Mannosyl (alpha-1,6-)- | Hs.22689 | 1E−160 | 87.03703704 |
| (SEQ ID NO: 2211) | | glycoprotein beta-1,6-N-acetyl- | | | |
| | | glucosaminyltransferase | | | |
| AF306662_at | MMP14 | Matrix metallopeptidase 14 | Hs.2399 | 0 | 89.7260274 |
| (SEQ ID NO: 1729) | | (membrane-inserted) | | | |
| WAN013I63_at | RPS15 | Ribosomal protein S15 | Hs.406683 | 1E−109 | 92.15017065 |
| (SEQ ID NO: 2212) | | | | | |
| WAN008BRK_at | Tmsb4x | Thymosin, beta 4, X | Hs.522584 | 1E−153 | 93.26683292 |
| (SEQ ID NO: 1594) | | chromosome | | | |
| WAN013I9L_x_at | UBB | Ubiquitin B | Hs.356190 | 3E−22 | 91.17647059 |
| (SEQ ID NO: 2213) | | | | | |

| | | Mouse | | | |
|---|---|---|---|---|---|
| Qualifier List | % QC | Unigene ID | eValue | % ID | % QC |
| WAN013I0S_at | 99.25373134 | Mm.152466 | 1E−104 | 92.91044776 | 100 |
| (SEQ ID NO: 2209) | | | | | |
| WAN0088WF_at | 55.67226891 | Mm.224233 | 1E−113 | 94.40298507 | 56.30252101 |
| (SEQ ID NO: 1874) | | | | | |
| WAN008EVU_x_at | 59.63636364 | Mm.142872 | 1E−170 | 90.58380414 | 96.54545455 |
| (SEQ ID NO: 1887) | | | | | |
| X51747_at | 50.53191489 | Mm.13849 | 0 | 91.95171026 | 66.09042553 |
| (SEQ ID NO: 1587) | | | | | |
| WAN008D3S_x_at | 29.14572864 | Mm.28366 | 8E−20 | 90.69767442 | 43.2160804 |
| (SEQ ID NO: 2210) | | | | | |
| U62587_at | 100 | Mm.214682 | 0 | 92.92929293 | 100 |
| (SEQ ID NO: 2211) | | | | | |
| AF306662_at | 99.82905983 | Mm.280175 | 0 | 90.08547009 | 100 |
| (SEQ ID NO: 1729) | | | | | |
| WAN013I63_at | 61.04166667 | Mm.643 | 1E−108 | 92.5795053 | 58.95833333 |
| (SEQ ID NO: 2212) | | | | | |
| WAN008BRK_at | 76.52671756 | Mm.142729 | 0 | 95.53398058 | 98.28244275 |
| (SEQ ID NO: 1594) | | | | | |
| WAN013I9L_x_at | 21.07438017 | Mm.282093 | 4E−26 | 90.47619048 | 21.69421488 |
| (SEQ ID NO: 2213) | | | | | |

TABLE 28
HCGR 2 + 3 Overlap 1.2F DOWN (115)

| | | | Human | | |
|---|---|---|---|---|---|
| | | | Unigene | | |
| Qualifier List | Symbol | Title | ID | eValue | % ID |
| WAN008CPA_at | 1200007-B05Rik | RIKEN cDNA 1200007B05 gene | #N/A | 9E−84 | 92.79279279 |
| (SEQ ID NO: 2214) | | | | | |
| WAN008DFA_at | 2310042-G06Rik | RIKEN cDNA 2310042G06 gene | #N/A | 1E−163 | 88.51851852 |
| (SEQ ID NO: 2215) | | | | | |
| WAN008DI2_at | 2510049-I19Rik | RIKEN cDNA 2510049I19 gene | #N/A | 3E−65 | 91.97860963 |
| (SEQ ID NO: 2216) | | | | | |
| WAN008D0K_at | AA408296 | Expressed sequence AA408296 | #N/A | 1E−110 | 86.774942 |
| (SEQ ID NO: 1877) | | | | | |
| WAN008CW0_at | AKAP12 | A kinase (PRKA) anchor protein | Hs.371240 | 2E−14 | 85.57692308 |
| (SEQ ID NO: 2217) | | (gravin) 12 | | | |
| WAN013I9E_at | Akr1b8 | Aldo-keto reductase family 1, | #N/A | 1E−136 | 87.52556237 |
| (SEQ ID NO: 2218) | | member B8 | | | |
| WAN0088LT_at | ALAS1 | Aminolevulinate, delta-, synthase 1 | Hs.476308 | 7E−95 | 85.8190709 |
| (SEQ ID NO: 2219) | | | | | |
| WAN008DGD_at | Aplp2 | Amyloid beta (A4) precursor-like | Hs.370247 | 0 | 0 |
| (SEQ ID NO: 1564) | | protein 2 | | | |
| WAN013I8H_x_at | APP | Amyloid beta (A4) precursor protein | Hs.642685 | 1E−77 | 83.89830508 |
| (SEQ ID NO: 1548) | | (peptidase nexin-II, Alzheimer | | | |
| | | disease) | | | |
| WAN0088T2_at | ATF4 | Activating transcription factor 4 (tax- | Hs.496487 | 1E−158 | 88.53974122 |
| (SEQ ID NO: 1592) | | responsive enhancer element B67) | | | |
| WAN008DS1_at | ATXN10 | Ataxin 10 | Hs.475125 | 1E−107 | 87.05035971 |
| (SEQ ID NO: 2220) | | | | | |
| WAN0088OB_at | BC017158 | CDNA sequence BC017158 | #N/A | 2E−69 | 84.3575419 |
| (SEQ ID NO: 2221) | | | | | |
| WAN013HXN_at | CAT | Catalase | Hs.502302 | 2E−71 | 92.74611399 |
| (SEQ ID NO: 2222) | | | | | |
| WAN008DU8_at | CCM2 | Cerebral cavernous malformation 2 | Hs.148272 | 1E−139 | 93.10344828 |
| (SEQ ID NO: 2223) | | | | | |
| WAN013I8J_at | CCNB2 | Cyclin B2 | Hs.194698 | 1E−173 | 86.92579505 |
| (SEQ ID NO: 2001) | | | | | |
| WAN0088YD_at | CCS | Copper chaperone for superoxide | Hs.502917 | 4E−96 | 83.89662028 |
| (SEQ ID NO: 2224) | | dismutase | | | |
| U42430_at | CD36 | CD36 molecule (thrombospondin | Hs.120949 | 6E−43 | 86.36363636 |
| (SEQ ID NO: 1673) | | receptor) | | | |
| WAN013HYB_at | CLIC1 | Chloride intracellular channel 1 | Hs.414565 | 1E−114 | 87.5 |
| (SEQ ID NO: 2225) | | | | | |
| WAN008D27_at | CLTA | Clathrin, light polypeptide (Lca) | Hs.522114 | 0 | 94.06307978 |
| (SEQ ID NO: 1760) | | | | | |
| WAN0088NS_at | CNOT6L | CCR4-NOT transcription complex, | Hs.591695 | 1E−113 | 97.43589744 |
| (SEQ ID NO: 2226) | | subunit 6-like | | | |
| WAN008CST_at | COPS2 | COP9 constitutive | Hs.369614 | 0 | 94.16058394 |
| (SEQ ID NO: 1711) | | photomorphogenic homolog subunit | | | |
| | | 2 (Arabidopsis) | | | |
| WAN008EEB_at | CORO1B | Coronin, actin binding protein, 1B | Hs.6191 | 2E−95 | 90.34482759 |
| (SEQ ID NO: 1762) | | | | | |
| AY011645_at | CREM | CAMP responsive element | Hs.200250 | 9E−44 | 94.02985075 |
| (SEQ ID NO: 2227) | | modulator | | | |
| WAN013I6I_at | CRYAB | Crystallin, alpha B | Hs.408767 | 1E−113 | 86.59793814 |
| (SEQ ID NO: 2228) | | | | | |
| WAN008DS3_at | CTNNBL1 | Catenin, beta like 1 | Hs.472667 | 1E−167 | 89.05660377 |
| (SEQ ID NO: 2229) | | | | | |
| WAN013I21_at | Cycs | Cytochrome c, somatic | Hs.437060 | 1E−103 | 89.21832884 |
| (SEQ ID NO: 2230) | | | | | |
| M29238_at | DDIT3 | DNA-damage-inducible transcript 3 | Hs.505777 | 1E−100 | 87.08971554 |
| (SEQ ID NO: 2030) | | | | | |
| WAN008ERF_at | DHCR7 | 7-dehydrocholesterol reductase | Hs.503134 | 2E−27 | 79.9382716 |
| (SEQ ID NO: 2231) | | | | | |
| WAN013HUM_at | EHD4 | EH-domain containing 4 | Hs.143703 | 1E−95 | 92.77108434 |
| (SEQ ID NO: 1576) | | | | | |
| WAN008E8R_at | EIF3S1 | Eukaryotic translation initiation factor | Hs.404056 | 1E−122 | 93.1372549 |
| (SEQ ID NO: 1767) | | 3, subunit 1 alpha, 35 kDa | | | |
| WAN008EJ7_at | EIF5A | Eukaryotic translation initiation factor | Hs.534314 | 0 | 98.52941176 |
| (SEQ ID NO: 2026) | | 5A | | | |
| WAN008EC0_at | EIF5B | Eukaryotic translation initiation factor | Hs.158688 | 5E−85 | 89.28571429 |
| (SEQ ID NO: 2232) | | 5B | | | |
| WAN013HYP_at | FEM1A | Fem-1 homolog a (C. elegans) | Hs.515082 | 9E−84 | 86.66666667 |
| (SEQ ID NO: 2233) | | | | | |
| Y12837_at | Fxr1h | Fragile X mental retardation gene 1, | #N/A | 0 | 96.12756264 |
| (SEQ ID NO: 2120) | | autosomal homolog | | | |
| M60973_at | GADD45A | Growth arrest and DNA-damage- | Hs.80409 | 0 | 91.57372986 |
| (SEQ ID NO: 2031) | | inducible, alpha | | | |
| WAN013I39_at | Gga2 | Golgi associated, gamma adaptin | Hs.460336 | 7E−30 | 84.45378151 |
| (SEQ ID NO: 1541) | | ear containing, ARF binding protein 2 | | | |
| WAN013I4Q_at | GLUL | Glutamate-ammonia ligase | Hs.518525 | 1E−136 | 88.44221106 |
| (SEQ ID NO: 2234) | | (glutamine synthetase) | | | |
| WAN013I0X_at | GSS | Glutathione synthetase | Hs.82327 | 1E−98 | 90.78498294 |
| (SEQ ID NO: 1581) | | | | | |
| WAN008EPH_at | GTF2H4 | General transcription factor IIH, | Hs.485070 | 1E−111 | 88.97435897 |
| (SEQ ID NO: 2235) | | polypeptide 4, 52 kDa | | | |
| WAN008E8M_at | HADHB | Hydroxyacyl-Coenzyme A | Hs.534639 | 1E−114 | 88.19095477 |
| (SEQ ID NO: 1568) | | dehydrogenase/3-ketoacyl- | | | |
| | | Coenzyme A thiolase/enoyl- | | | |
| | | Coenzyme A hydratase (trifunctional | | | |
| | | protein), beta subunit | | | |
| WAN008EKL_at | HBP1 | HMG-box transcription factor 1 | Hs.162032 | 1E−120 | 89.60784314 |
| (SEQ ID NO: 1775) | | | | | |
| WAN008CWV_at | HDGF | Hepatoma-derived growth factor | Hs.506748 | 3E−75 | 91.4893617 |
| (SEQ ID NO: 1684) | | (high-mobility group protein 1-like) | | | |
| WAN0088XH_at | HERPUD1 | Homocysteine-inducible, | Hs.146393 | 7E−79 | 87.41721854 |
| (SEQ ID NO: 1557) | | endoplasmic reticulum stress- | | | |
| | | inducible, ubiquitin-like domain | | | |
| | | member 1 | | | |
| L00180_at | Hmgcr | 3-hydroxy-3-methylglutaryl- | Hs.643495 | 5E−35 | 86.30952381 |
| (SEQ ID NO: 1509) | | Coenzyme A reductase | | | |
| WAN008D6E_x_at | HMOX2 | Heme oxygenase (decycling) 2 | Hs.284279 | 2E−32 | 94.50549451 |
| (SEQ ID NO: 2236) | | | | | |
| WAN008CLS_at | HSDL2 | Hydroxysteroid dehydrogenase like 2 | Hs.59486 | 1E−148 | 87.09090909 |
| (SEQ ID NO: 2237) | | | | | |
| WAN013I8Z_at | Hspa8 | Heat shock protein 8 | Hs.180414 | 0 | 90.56603774 |
| (SEQ ID NO: 2238) | | | | | |
| WAN008D42_at | HSPB8 | Heat shock 22 kDa protein 8 | Hs.400095 | 1E−16 | 89.02439024 |
| (SEQ ID NO: 2239) | | | | | |
| WAN013I26_at | IDH3A | Isocitrate dehydrogenase 3 (NAD+) | Hs.591110 | 0 | 89.26746167 |
| (SEQ ID NO: 2114) | | alpha | | | |
| WAN008EX2_x_at | Ifrd1 | Interferon-related developmental | Hs.7879 | 7E−39 | 90.29850746 |
| (SEQ ID NO: 1575) | | regulator 1 | | | |
| WAN013I0I_at | INSIG1 | Insulin induced gene 1 | Hs.520819 | 4E−21 | 94.20289855 |
| (SEQ ID NO: 2240) | | | | | |
| WAN0088O9_at | Itgb1 | Integrin beta 1 (fibronectin receptor | Hs.295626 | 1E−13 | 90.32258065 |
| (SEQ ID NO: 1777) | | beta) | | | |
| AF081140_at | Itgb4bp | Integrin beta 4 binding protein | Hs.632277 | 0 | 0 |
| (SEQ ID NO: 2241) | | | | | |
| X83575_at | KIF23 | Kinesin family member 23 | Hs.270845 | 1E−177 | 92.47787611 |
| (SEQ ID NO: 2007) | | | | | |
| WAN008D29_s_at | LGALS3 | Lectin, galactoside-binding, soluble, | Hs.531081 | 3E−41 | 91.8128655 |
| (SEQ ID NO: 2242) | | 3 (galectin 3) | | | |
| WAN008DBR_at | LUC7L | LUC7-like (S. cerevisiae) | Hs.16803 | 0 | 93.66197183 |
| (SEQ ID NO: 1966) | | | | | |
| WAN008ECL_at | MAN2C1 | Mannosidase, alpha, class 2C, | Hs.26232 | 1E−134 | 87.68267223 |
| (SEQ ID NO: 2243) | | member 1 | | | |
| WAN008E7Y_at | MCM7 | MCM7 minichromosome | Hs.438720 | 1E−96 | 84.53389831 |
| (SEQ ID NO: 2244) | | maintenance deficient 7 (S. cerevisiae) | | | |
| WAN013I23_at | MORF4L2 | Mortality factor 4 like 2 | Hs.326387 | 0 | 89.13043478 |
| (SEQ ID NO: 2245) | | | | | |
| WAN008CRR_at | MRPL19 | Mitochondrial ribosomal protein L19 | Hs.44024 | 1E−71 | 86.89655172 |
| (SEQ ID NO: 2246) | | | | | |
| WAN013I02_at | MRPS18A | Mitochondrial ribosomal protein | Hs.520149 | 1E−137 | 85.78767123 |
| (SEQ ID NO: 2247) | | S18A | | | |
| WAN008EV8_at | MTHFD2 | Methylenetetrahydrofolate | Hs.469030 | 7E−50 | 88.35978836 |
| (SEQ ID NO: 2189) | | dehydrogenase (NADP+ dependent) | | | |
| | | 2, methenyltetrahydrofolate | | | |
| | | cyclohydrolase | | | |
| WAN008EQD_at | Mx2 | Myxovirus (influenza virus) | Hs.926 | 2E−29 | 80.31746032 |
| (SEQ ID NO: 1894) | | resistance 2 | | | |
| WAN008BSP_x_at | NDFIP1 | Nedd4 family interacting protein 1 | Hs.9788 | 5E−33 | 92.23300971 |
| (SEQ ID NO: 2248) | | | | | |
| WAN008EDO_at | NDRG3 | NDRG family member 3 | Hs.437338 | 5E−86 | 88.00904977 |
| (SEQ ID NO: 2249) | | | | | |
| WAN013I17_at | NID1 | Nidogen 1 | Hs.356624 | 1E−101 | 83.47826087 |
| (SEQ ID NO: 1790) | | | | | |
| AF182744_at | NPC1 | Niemann-Pick disease, type C1 | Hs.464779 | 6E−83 | 85.41114058 |
| (SEQ ID NO: 2250) | | | | | |
| WAN008EK9_at | Nup160 | Nucleoporin 160 | Hs.645358 | 1E−159 | 89.2 |
| (SEQ ID NO: 2251) | | | | | |
| WAN0088ZP_at | Pawr | PRKC, apoptosis, WT1, regulator | Hs.643130 | 4E−10 | 91.52542373 |
| (SEQ ID NO: 1482) | | | | | |
| WAN008EML_at | PBK | PDZ binding kinase | Hs.104741 | 5E−52 | 89.50276243 |
| (SEQ ID NO: 1980) | | | | | |
| WAN0088X2_at | PEO1 | Progressive external | Hs.22678 | 1E−141 | 88.6509636 |
| (SEQ ID NO: 1593) | | ophthalmoplegia 1 | | | |
| WAN013I38_at | Pkm2 | Pyruvate kinase, muscle | Hs.534770 | 4E−13 | 92.85714286 |
| (SEQ ID NO: 1583) | | | | | |
| WAN0088KG_at | PPGB | Protective protein for beta- | Hs.517076 | 1E−115 | 87.58949881 |
| (SEQ ID NO: 1539) | | galactosidase (galactosialidosis) | | | |
| WAN008968_at | PPP4R1 | Protein phosphatase 4, regulatory | Hs.464595 | 1E−141 | 86.24338624 |
| (SEQ ID NO: 2252) | | subunit 1 | | | |
| WAN008D4W_at | PPT2 | Palmitoyl-protein thioesterase 2 | Hs.332138 | 6E−82 | 86.29737609 |
| (SEQ ID NO: 2079) | | | | | |
| WAN0088ZC_at | PSEN1 | Presenilin 1 (Alzheimer disease 3) | Hs.592324 | 5E−82 | 89.16083916 |
| (SEQ ID NO: 1543) | | | | | |
| WAN013HXU_at | PSMA1 | Proteasome (prosome, macropain) | Hs.102798 | 0 | 92.22011385 |
| (SEQ ID NO: 2156) | | subunit, alpha type, 1 | | | |
| WAN013HVR_at | PSMD4 | Proteasome (prosome, macropain) | Hs.505059 | 0 | 90.23090586 |
| (SEQ ID NO: 2253) | | 26S subunit, non-ATPase, 4 | | | |
| WAN008CLK_at | RAB6A | RAB6A, member RAS oncogene | Hs.12152 | 2E−55 | 88.47736626 |
| (SEQ ID NO: 1552) | | family | | | |
| WAN008CWE_at | Ralb | V-ral simian leukemia viral oncogene | Hs.469820 | 0 | 0 |
| (SEQ ID NO: 2254) | | homolog B (ras related) | | | |
| WAN008DNA_at | RBM28 | RNA binding motif protein 28 | Hs.274263 | 2E−76 | 86.62420382 |
| (SEQ ID NO: 2255) | | | | | |
| X61588_at | RHOG | Ras homolog gene family, member | Hs.501728 | 0 | 90.47619048 |
| (SEQ ID NO: 2256) | | G (rho G) | | | |
| WAN013HVB_at | RNF10 | Ring finger protein 10 | Hs.442798 | 1E−180 | 92.22462203 |
| (SEQ ID NO: 1915) | | | | | |
| WAN008CQ7_at | ROD1 | ROD1 regulator of differentiation 1 | Hs.269988 | 4E−84 | 96.31336406 |
| (SEQ ID NO: 2257) | | (S. pombe) | | | |
| WAN008EE3_at | SARS | Seryl-tRNA synthetase | Hs.531176 | 1E−116 | 90.80118694 |
| (SEQ ID NO: 1809) | | | | | |
| WAN008ENH_at | SCAMP5 | Secretory carrier membrane protein 5 | Hs.374180 | 1E−173 | 96.38888889 |
| (SEQ ID NO: 2258) | | | | | |
| WAN013I6D_at | Sdc1 | Syndecan 1 | Hs.224607 | 1E−26 | 90 |
| (SEQ ID NO: 2259) | | | | | |
| WAN008EHX_at | Setd8 | SET domain containing (lysine | Hs.572262 | 1E−141 | 86.65447898 |
| (SEQ ID NO: 1920) | | methyltransferase) 8 | | | |
| WAN008DJ9_at | SLC1A4 | Solute carrier family 1 | Hs.323878 | 1E−34 | 86.36363636 |
| (SEQ ID NO: 1565) | | (glutamate/neutral amino acid | | | |
| | | transporter), member 4 | | | |
| WAN013I1G_at | SLC25A20 | Solute carrier family 25 | Hs.13845 | 1E−137 | 86.70634921 |
| (SEQ ID NO: 1582) | | (carnitine/acylcarnitine translocase), | | | |
| | | member 20 | | | |
| WAN00895Z_at | SNAP29 | Synaptosomal-associated protein, | Hs.108002 | 2E−84 | 85.95041322 |
| (SEQ ID NO: 2260) | | 29 kDa | | | |
| WAN008EFY_at | SPG21 | Spastic paraplegia 21 (autosomal | Hs.242458 | 1E−128 | 90.45092838 |
| (SEQ ID NO: 1811) | | recessive, Mast syndrome) | | | |
| WAN008EBP_at | SQSTM1 | Sequestosome 1 | Hs.437277 | 1E−166 | 89.36170213 |
| (SEQ ID NO: 1621) | | | | | |
| WAN0088TG_at | SRP72 | Signal recognition particle 72 kDa | Hs.237825 | 1E−58 | 89.04109589 |
| (SEQ ID NO: 1540) | | | | | |
| WAN008EH6_at | STT3A | STT3, subunit of the | Hs.504237 | 0 | 93.63636364 |
| (SEQ ID NO: 2141) | | oligosaccharyltransferase complex, | | | |
| | | homolog A (S. cerevisiae) | | | |
| WAN013I0W_at | TAPBP | TAP binding protein (tapasin) | Hs.370937 | 2E−57 | 80.63314711 |
| (SEQ ID NO: 1580) | | | | | |
| WAN013HX6_at | TAX1BP1 | Tax1 (human T-cell leukemia virus | Hs.34576 | 1E−111 | 86.72985782 |
| (SEQ ID NO: 1815) | | type I) binding protein 1 | | | |
| WAN008F02_at | TFPI | Tissue factor pathway inhibitor | Hs.516578 | 3E−14 | 83.33333333 |
| (SEQ ID NO: 2191) | | (lipoprotein-associated coagulation | | | |
| | | inhibitor) | | | |
| WAN008EAE_at | Tloc1 | Translocation protein 1 | Hs.592561 | 1E−132 | 92.8358209 |
| (SEQ ID NQ: 2261) | | | | | |
| WAN008ER4_at | TMC6 | Transmembrane channel-like 6 | Hs.632227 | 3E−41 | 84.75336323 |
| (SEQ ID NO: 2262) | | | | | |
| WAN013I1C_at | Trib3 | Tribbles homolog 3 (Drosophila) | Hs.516826 | 0 | 0 |
| (SEQ ID NO: 2263) | | | | | |
| WAN008DPJ_at | Trim26 | Tripartite motif protein 26 | Hs.485041 | 0 | 0 |
| (SEQ ID NO: 2264) | | | | | |
| WAN008CVL_x_at | TUBG1 | Tubulin, gamma 1 | Hs.279669 | 8E−64 | 93.90243902 |
| (SEQ ID NO: 2265) | | | | | |
| WAN008CYV_at | TXNDC4 | Thioredoxin domain containing 4 | Hs.591899 | 4E−99 | 93.85245902 |
| (SEQ ID NO: 2266) | | (endoplasmic reticulum) | | | |
| WAN0088P2_at | UAP1 | UDP-N-acteylglucosamine | Hs.492859 | 1E−130 | 86.73267327 |
| (SEQ ID NO: 1853) | | pyrophosphorylase 1 | | | |
| WAN008E3E_at | UBE2G2 | Ubiquitin-conjugating enzyme E2G 2 | Hs.529420 | 1E−168 | 90.75630252 |
| (SEQ ID NO: 2267) | | (UBC7 homolog, yeast) | | | |
| WAN008EA0_at | VCP | Valosin-containing protein | Hs.529782 | 0 | 95.18348624 |
| (SEQ ID NO: 1487) | | | | | |
| WAN008D19_x_at | Vps35 | Vacuolar protein sorting 35 | Hs.454528 | 2E−55 | 95.52238806 |
| (SEQ ID NO: 2268) | | | | | |
| WAN013I0V_at | WBP2 | WW domain binding protein 2 | Hs.514489 | 1E−35 | 90.32258065 |
| (SEQ ID NO: 2269) | | | | | |
| WAN013I07_at | WDR43 | WD repeat domain 43 | Hs.169863 | 3E−47 | 92.02898551 |
| (SEQ ID NO: 2270) | | | | | |
| WAN008ES6_at | XAB1 | XPA binding protein 1, GTPase | Hs.18259 | 1E−128 | 88.70588235 |
| (SEQ ID NO: 2271) | | | | | |
| WAN008DQM_at | XPNPEP1 | X-prolyl aminopeptidase | Hs.390623 | 1E−147 | 88.11881188 |
| (SEQ ID NO: 2181) | | (aminopeptidase P) 1, soluble | | | |
| WAN008DNP_at | XPO1 | Exportin 1 (CRM1 homolog, yeast) | Hs.370770 | 0 | 91.08527132 |
| (SEQ ID NO: 1876) | | | | | |
| WAN0088PU_at | Ywhab | Tyrosine 3- | Hs.645387 | 1E−102 | 92.61538462 |
| (SEQ ID NO: 2128) | | monooxygenase/tryptophan 5- | | | |
| | | monooxygenase activation protein, | | | |
| | | beta polypeptide | | | |

| | | Mouse | | | |
|---|---|---|---|---|---|
| Qualifier List | % QC | Unigene ID | eValue | % ID | % QC |
| WAN008CPA_at | 61.66666667 | Mm.23896 | 1E−117 | 91.5451895 | 95.27777778 |
| (SEQ ID NO: 2214) | | | | | |
| WAN008DFA_at | 97.12230216 | Mm.182294 | 0 | 93.22344322 | 98.20143885 |
| (SEQ ID NO: 2215) | | | | | |
| WAN008DI2_at | 60.12861736 | Mm.28327 | 2E−95 | 92.20779221 | 99.03536977 |
| (SEQ ID NO: 2216) | | | | | |
| WAN008D0K_at | 96.85393258 | Mm.173758 | 1E−143 | 89.86486486 | 99.7752809 |
| (SEQ ID NO: 1877) | | | | | |
| WAN008CW0_at | 25.93516209 | Mm.27481 | 2E−37 | 90.44117647 | 33.91521197 |
| (SEQ ID NO: 2217) | | | | | |
| WAN013I9E_at | 43.00791557 | Mm.5378 | 0 | 89.56989247 | 81.79419525 |
| (SEQ ID NO: 2218) | | | | | |
| WAN0088LT_at | 76.5917603 | Mm.290578 | 1E−166 | 91.52892562 | 90.63670412 |
| (SEQ ID NO: 2219) | | | | | |
| WAN008DGD_at | 0 | Mm.19133 | 6E−69 | 93.0875576 | 44.46721311 |
| (SEQ ID NO: 1564) | | | | | |
| WAN013I8H_x_at | 83.53982301 | Mm.277585 | 1E−167 | 87.78761062 | 100 |
| (SEQ ID NO: 1548) | | | | | |
| WAN0088T2_at | 97.83001808 | Mm.641 | 0 | 91.71374765 | 96.02169982 |
| (SEQ ID NO: 1592) | | | | | |
| WAN008DS1_at | 82.24852071 | Mm.248906 | 1E−170 | 91.32149901 | 100 |
| (SEQ ID NO: 2220) | | | | | |
| WAN0088OB_at | 99.44444444 | Mm.38870 | 1E−142 | 92.77777778 | 100 |
| (SEQ ID NO: 2221) | | | | | |
| WAN013HXN_at | 33.80035026 | Mm.4215 | 0 | 90.03831418 | 91.41856392 |
| (SEQ ID NO: 2222) | | | | | |
| WAN008DU8_at | 75.16198704 | Mm.221271 | 1E−162 | 95.23809524 | 77.10583153 |
| (SEQ ID NO: 2223) | | | | | |
| WAN013I8J_at | 44.39215686 | Mm.22592 | 0 | 90.70945946 | 46.43137255 |
| (SEQ ID NO: 2001) | | | | | |
| WAN0088YD_at | 90.4676259 | Mm.426068 | 0.00005 | 96.66666667 | 5.395683453 |
| (SEQ ID NO: 2224) | | | | | |
| U42430_at | 34.25605536 | Mm.18628 | 2E−57 | 88.0733945 | 37.71626298 |
| (SEQ ID NO: 1673) | | | | | |
| WAN013HYB_at | 98.9010989 | Mm.29524 | 1E−130 | 89.28571429 | 100 |
| (SEQ ID NO: 2225) | | | | | |
| WAN008D27_at | 99.26335175 | Mm.298875 | 0 | 94.83394834 | 99.81583794 |
| (SEQ ID NO: 1760) | | | | | |
| WAN0088NS_at | 50.86956522 | Mm.28374 | 1E−106 | 98.13953488 | 46.73913043 |
| (SEQ ID NO: 2226) | | | | | |
| WAN008CST_at | 100 | Mm.3596 | 0 | 96.89781022 | 100 |
| (SEQ ID NO: 1711) | | | | | |
| WAN008EEB_at | 55.98455598 | Mm.276859 | 1E−128 | 94.61279461 | 57.33590734 |
| (SEQ ID NO: 1762) | | | | | |
| AY011645_at | 37.4301676 | Mm.5244 | 1E−149 | 93.82022472 | 99.44134078 |
| (SEQ ID NO: 2227) | | | | | |
| WAN013I6I_at | 97.97979798 | Mm.178 | 1E−124 | 86.80203046 | 99.49494949 |
| (SEQ ID NO: 2228) | | | | | |
| WAN008DS3_at | 99.06542056 | Mm.45193 | 0 | 90.8411215 | 100 |
| (SEQ ID NO: 2229) | | | | | |
| WAN013I21_at | 73.3201581 | Mm.35389 | 0 | 95.86956522 | 90.90909091 |
| (SEQ ID NO: 2230) | | | | | |
| M29238_at | 76.80672269 | Mm.110220 | 1E−119 | 89.26829268 | 68.90756303 |
| (SEQ ID NO: 2030) | | | | | |
| WAN008ERF_at | 70.74235808 | Mm.249342 | 1E−147 | 90.02217295 | 98.47161572 |
| (SEQ ID NO: 2231) | | | | | |
| WAN013HUM_at | 59.00473934 | Mm.132226 | 1E−125 | 88.57142857 | 99.52606635 |
| (SEQ ID NO: 1576) | | | | | |
| WAN008E8R_at | 71.1627907 | Mm.27695 | 1E−140 | 95.46925566 | 71.86046512 |
| (SEQ ID NO: 1767) | | | | | |
| WAN008EJ7_at | 100 | Mm.196607 | 0 | 97.54901961 | 100 |
| (SEQ ID NO: 2026) | | | | | |
| WAN008EC0_at | 97.56097561 | Mm.260943 | 1E−100 | 91.45907473 | 97.90940767 |
| (SEQ ID NO: 2232) | | | | | |
| WAN013HYP_at | 58.61456483 | Mm.290813 | 1E−107 | 89.28571429 | 59.68028419 |
| (SEQ ID NO: 2233) | | | | | |
| Y12837_at | 100 | Mm.259021 | 0 | 97.72209567 | 100 |
| (SEQ ID NO: 2120) | | | | | |
| M60973_at | 76.34815516 | Mm.389750 | 1E−170 | 90.57692308 | 49.19583728 |
| (SEQ ID NO: 2031) | | | | | |
| WAN013I39_at | 46.66666667 | Mm.29619 | 1E−147 | 93.33333333 | 79.41176471 |
| (SEQ ID NO: 1541) | | | | | |
| WAN013I4Q_at | 74.39252336 | Mm.210745 | 1E−135 | 88.11881188 | 75.51401869 |
| (SEQ ID NO: 2234) | | | | | |
| WAN013I0X_at | 56.1302682 | Mm.252316 | 1E−129 | 95.18900344 | 55.74712644 |
| (SEQ ID NO: 1581) | | | | | |
| WAN008EPH_at | 87.64044944 | Mm.10182 | 1E−148 | 91.87817259 | 88.53932584 |
| (SEQ ID NO: 2235) | | | | | |
| WAN008E8M_at | 85.77586207 | Mm.291463 | 1E−160 | 91.03139013 | 96.12068966 |
| (SEQ ID NO: 1568) | | | | | |
| WAN008EKL_at | 98.45559846 | Mm.390461 | 0 | 91.6827853 | 99.80694981 |
| (SEQ ID NO: 1775) | | | | | |
| WAN008CWV_at | 43.04029304 | Mm.292208 | 2E−75 | 91.4893617 | 43.04029304 |
| (SEQ ID NO: 1684) | | | | | |
| WAN0088XH_at | 68.48072562 | Mm.29151 | 1E−151 | 91.46341463 | 92.97052154 |
| (SEQ ID NO: 1557) | | | | | |
| L00180_at | 68.01619433 | Mm.316652 | 1E−49 | 90.24390244 | 66.39676113 |
| (SEQ ID NO: 1509) | | | | | |
| WAN008D6E_x_at | 28.4375 | Mm.272866 | 2E−37 | 95.78947368 | 29.6875 |
| (SEQ ID NO: 2236) | | | | | |
| WAN008CLS_at | 95.65217391 | Mm.272905 | 0 | 93.01919721 | 99.65217391 |
| (SEQ ID NO: 2237) | | | | | |
| WAN013I8Z_at | 69.19060052 | Mm.290774 | 0 | 93.73368146 | 100 |
| (SEQ ID NO: 2238) | | | | | |
| WAN008D42_at | 15.3271028 | Mm.21549 | 3E−31 | 90.90909091 | 20.56074766 |
| (SEQ ID NO: 2239) | | | | | |
| WAN013I26_at | 98.16053512 | Mm.279195 | 0 | 94.06779661 | 98.66220736 |
| (SEQ ID NO: 2114) | | | | | |
| WAN008EX2_x_at | 100 | Mm.168 | 5E−63 | 97.76119403 | 100 |
| (SEQ ID NO: 1575) | | | | | |
| WAN013I0I_at | 12.94559099 | Mm.30221 | 3E−44 | 86.53846154 | 39.02439024 |
| (SEQ ID NO: 2240) | | | | | |
| WAN0088O9_at | 16.4893617 | Mm.263396 | 4E−55 | 90.49773756 | 39.18439716 |
| (SEQ ID NO: 1777) | | | | | |
| AF081140_at | 0 | Mm.271674 | 5E−56 | 92.02453988 | 52.24358974 |
| (SEQ ID NO: 2241) | | | | | |
| X83575_at | 37.07957342 | Mm.259374 | 0 | 91.99372057 | 52.2559475 |
| (SEQ ID NO: 2007) | | | | | |
| WAN008D29_s_at | 31.78438662 | Mm.248615 | 5E−79 | 88.48484848 | 61.33828996 |
| (SEQ ID NO: 2242) | | | | | |
| WAN008DBR_at | 100 | Mm.386921 | 0 | 95.07042254 | 100 |
| (SEQ ID NO: 1966) | | | | | |
| WAN008ECL_at | 96.96356275 | Mm.30110 | 1E−119 | 93.02325581 | 60.93117409 |
| (SEQ ID NO: 2243) | | | | | |
| WAN008E7Y_at | 88.38951311 | Mm.241714 | 1E−171 | 90.98532495 | 89.3258427 |
| (SEQ ID NO: 2244) | | | | | |
| WAN013I23_at | 96.61610268 | Mm.27218 | 0 | 92.6487748 | 100 |
| (SEQ ID NO: 2245) | | | | | |
| WAN008CRR_at | 58 | Mm.276293 | 1E−136 | 87.34939759 | 99.6 |
| (SEQ ID NO: 2246) | | | | | |
| WAN013I02_at | 89.02439024 | Mm.287443 | 0 | 90.92465753 | 89.02439024 |
| (SEQ ID NO: 2247) | | | | | |
| WAN008EV8_at | 68.23104693 | Mm.443 | 4E−84 | 91.05691057 | 88.80866426 |
| (SEQ ID NO: 2189) | | | | | |
| WAN008EQD_at | 75 | Mm.14157 | 2E−99 | 85.95238095 | 100 |
| (SEQ ID NO: 1894) | | | | | |
| WAN008BSP_x_at | 93.63636364 | Mm.102496 | 1E−44 | 95.45454545 | 100 |
| (SEQ ID NO: 2248) | | | | | |
| WAN008EDO_at | 92.85714286 | Mm.279256 | 3E−92 | 93.41563786 | 51.05042017 |
| (SEQ ID NO: 2249) | | | | | |
| WAN013I17_at | 83.03249097 | Mm.4691 | 9E−99 | 91.66666667 | 45.48736462 |
| (SEQ ID NO: 1790) | | | | | |
| AF182744_at | 70.20484171 | Mm.3484 | 1E−167 | 92.13483146 | 82.86778399 |
| (SEQ ID NO: 2250) | | | | | |
| WAN008EK9_at | 99.60159363 | Mm.24532 | 0 | 94.36619718 | 99.00398406 |
| (SEQ ID NO: 2251) | | | | | |
| WAN0088ZP_at | 11.11111111 | Mm.391419 | 8E−53 | 91.62561576 | 38.22975518 |
| (SEQ ID NO: 1482) | | | | | |
| WAN008EML_at | 39.09287257 | Mm.24337 | 3E−80 | 89.7810219 | 59.17926566 |
| (SEQ ID NO: 1980) | | | | | |
| WAN0088X2_at | 94.53441296 | Mm.105585 | 7E−78 | 90.6779661 | 47.77327935 |
| (SEQ ID NO: 1593) | | | | | |
| WAN013I38_at | 10.18181818 | Mm.326167 | 0 | 92.42718447 | 93.63636364 |
| (SEQ ID NO: 1583) | | | | | |
| WAN0088KG_at | 72.86956522 | Mm.359633 | 1E−149 | 90.93078759 | 72.86956522 |
| (SEQ ID NO: 1539) | | | | | |
| WAN008968_at | 99.47368421 | Mm.48686 | 0 | 91.34438306 | 95.26315789 |
| (SEQ ID NO: 2252) | | | | | |
| WAN008D4W_at | 97.44318182 | Mm.373627 | 1E−96 | 89.38906752 | 88.35227273 |
| (SEQ ID NO: 2079) | | | | | |
| WAN0088ZC_at | 86.14457831 | Mm.998 | 4E−78 | 88.1533101 | 86.44578313 |
| (SEQ ID NO: 1543) | | | | | |
| WAN013HXU_at | 100 | Mm.121265 | 0 | 94.84732824 | 99.43074004 |
| (SEQ ID NO: 2156) | | | | | |
| WAN013HVR_at | 100 | Mm.2261 | 0 | 94.12811388 | 99.82238011 |
| (SEQ ID NO: 2253) | | | | | |
| WAN008CLK_at | 48.21428571 | #N/A | 1E−161 | 92.07317073 | 97.61904762 |
| (SEQ ID NO: 1552) | | | | | |
| WAN008CWE_at | 0 | Mm.27832 | 2E−34 | 90.24390244 | 30.37037037 |
| (SEQ ID NO: 2254) | | | | | |
| WAN008DNA_at | 55.9714795 | Mm.40802 | 1E−172 | 90.38112523 | 98.21746881 |
| (SEQ ID NO: 2255) | | | | | |
| X61588_at | 67.5 | Mm.259795 | 0 | 93.49693252 | 72.76785714 |
| (SEQ ID NO: 2256) | | | | | |
| WAN013HVB_at | 80.66202091 | Mm.30051 | 1E−158 | 89.56158664 | 83.44947735 |
| (SEQ ID NO: 1915) | | | | | |
| WAN008CQ7_at | 39.74358974 | Mm.331640 | 1E−121 | 97.31800766 | 47.8021978 |
| (SEQ ID NO: 2257) | | | | | |
| WAN008EE3_at | 100 | Mm.28688 | 1E−136 | 93.17507418 | 100 |
| (SEQ ID NO: 1809) | | | | | |
| WAN008ENH_at | 79.47019868 | Mm.102278 | 0 | 95.58498896 | 100 |
| (SEQ ID NO: 2258) | | | | | |
| WAN013I6D_at | 9.615384615 | Mm.2580 | 0 | 88.17073171 | 60.65088757 |
| (SEQ ID NO: 2259) | | | | | |
| WAN008EHX_at | 100 | Mm.137966 | 0 | 89.76234004 | 100 |
| (SEQ ID NO: 1920) | | | | | |
| WAN008DJ9_at | 39.46188341 | Mm.6379 | 1E−119 | 88.36633663 | 90.58295964 |
| (SEQ ID NO: 1565) | | | | | |
| WAN013I1G_at | 87.65217391 | Mm.29666 | 0 | 92.35412475 | 86.43478261 |
| (SEQ ID NO: 1582) | | | | | |
| WAN00895Z_at | 100 | Mm.271992 | 3E−88 | 87.87878788 | 100 |
| (SEQ ID NO: 2260) | | | | | |
| WAN008EFY_at | 71.94656489 | Mm.272475 | 1E−136 | 91.44385027 | 71.3740458 |
| (SEQ ID NO: 1811) | | | | | |
| WAN008EBP_at | 92.65232975 | Mm.40828 | 0 | 93.40659341 | 97.84946237 |
| (SEQ ID NO: 1621) | | | | | |
| WAN0088TG_at | 50.81206497 | Mm.296976 | 1E−119 | 92.40121581 | 76.33410673 |
| (SEQ ID NO: 1540) | | | | | |
| WAN008EH6_at | 97.86476868 | Mm.2863 | 0 | 95.27272727 | 97.86476868 |
| (SEQ ID NO: 2141) | | | | | |
| WAN013I0W_at | 93.22916667 | Mm.154457 | 1E−149 | 86.8852459 | 95.3125 |
| (SEQ ID NO: 1580) | | | | | |
| WAN013HX6_at | 41.49459194 | Mm.431979 | 1E−124 | 89.42065491 | 39.03638151 |
| (SEQ ID NO: 1815) | | | | | |
| WAN008F02_at | 21.79930796 | Mm.124316 | 3E−83 | 85.5227882 | 64.53287197 |
| (SEQ ID NO: 2191) | | | | | |
| WAN008EAE_at | 100 | Mm.26017 | 1E−158 | 96.11940299 | 100 |
| (SEQ ID NQ: 2261) | | | | | |
| WAN008ER4_at | 42.31499051 | Mm.286963 | 1E−94 | 87.08791209 | 69.07020873 |
| (SEQ ID NO: 2262) | | | | | |
| WAN013I1C_at | 0 | Mm.276018 | 5E−22 | 92.20779221 | 13.02876481 |
| (SEQ ID NO: 2263) | | | | | |
| WAN008DPJ_at | 0 | Mm.34587 | 7E−10 | 89.18918919 | 14.82965932 |
| (SEQ ID NO: 2264) | | | | | |
| WAN008CVL_x_at | 96.47058824 | Mm.142348 | 2E−75 | 96.40718563 | 98.23529412 |
| (SEQ ID NO: 2265) | | | | | |
| WAN008CYV_at | 48.70259481 | Mm.317701 | 1E−142 | 95.76547231 | 61.27744511 |
| (SEQ ID NO: 2266) | | | | | |
| WAN0088P2_at | 91.98542805 | Mm.27969 | 1E−177 | 90.01883239 | 96.72131148 |
| (SEQ ID NO: 1853) | | | | | |
| WAN008E3E_at | 94.07114625 | Mm.307906 | 0 | 95.20958084 | 99.01185771 |
| (SEQ ID NO: 2267) | | | | | |
| WAN008EA0_at | 78.98550725 | Mm.262053 | 0 | 95.47101449 | 100 |
| (SEQ ID NO: 1487) | | | | | |
| WAN008D19_x_at | 86.4516129 | Mm.296520 | 1E−64 | 97.16312057 | 90.96774194 |
| (SEQ ID NO: 2268) | | | | | |
| WAN013I0V_at | 49.20634921 | Mm.284792 | 3E−56 | 87.55364807 | 92.46031746 |
| (SEQ ID NO: 2269) | | | | | |
| WAN013I07_at | 25.32110092 | Mm.257762 | 1E−63 | 90 | 38.53211009 |
| (SEQ ID NO: 2270) | | | | | |
| WAN008ES6_at | 98.60788863 | Mm.348649 | 1E−168 | 92.6713948 | 98.14385151 |
| (SEQ ID NO: 2271) | | | | | |
| WAN008DQM_at | 99.60552268 | Mm.99776 | 0 | 92.50493097 | 100 |
| (SEQ ID NO: 2181) | | | | | |
| WAN008DNP_at | 98.47328244 | Mm.217547 | 0 | 94.84732824 | 100 |
| (SEQ ID NO: 1876) | | | | | |
| WAN0088PU_at | 62.74131274 | Mm.34319 | 0 | 92.81553398 | 99.42084942 |
| (SEQ ID NO: 2128) | | | | | |

TABLE 29
LLP2 + 4 Test-specific 1.2F UP

| | | | Human | | |
|---|---|---|---|---|---|
| | | | Unigene | | |
| Qualifier List | Symbol | Title | ID | eValue | % ID |
| WAN013I76_at | ERBB2 | V-erb-b2 erythroblastic leukemia viral | Hs.446352 | 7E−61 | 87.23404255 |
| (SEQ ID | | oncogene homolog 2, | | | |
| NO: 2272) | | neuro/glioblastoma derived | | | |
| | | oncogene homolog (avian) | | | |
| WAN008BXV_at | Fbxl5 | F-box and leucine-rich repeat protein 5 | Hs.645220 | 4E−99 | 94.82758621 |
| (SEQ ID | | | | | |
| NO: 2273) | | | | | |
| WAN013I4Q_at | GLUL | Glutamate-ammonia ligase | Hs.518525 | 1E−136 | 88.44221106 |
| (SEQ ID | | (glutamine synthetase) | | | |
| NO: 2234) | | | | | |
| WAN008EMJ_at | GPC6 | Glypican 6 | Hs.444329 | 7E−64 | 92.55319149 |
| (SEQ ID | | | | | |
| NO: 1721) | | | | | |
| U73375_at | LGALS3BP | Lectin, galactoside-binding, soluble, | Hs.514535 | 5E−31 | 82.88288288 |
| (SEQ ID | | 3 binding protein | | | |
| NO: 2274) | | | | | |
| WAN013I2D_at | RAP2C | RAP2C, member of RAS oncogene | Hs.119889 | 4E−77 | 91.18942731 |
| (SEQ ID | | family | | | |
| NO: 2275) | | | | | |
| WAN0088ZJ_at | Slc4a2 | Solute carrier family 4 (anion | #N/A | 1E−162 | 88.57677903 |
| (SEQ ID | | exchanger), member 2 | | | |
| NO: 2075) | | | | | |

| | | Mouse | | | |
|---|---|---|---|---|---|
| Qualifier List | % QC | Unigene ID | eValue | % ID | % QC |
| WAN013I76_at | 51.55393053 | Mm.290822 | 5E−76 | 88.54166667 | 52.65082267 |
| (SEQ ID | | | | | |
| NO: 2272) | | | | | |
| WAN008BXV_at | 43.04267161 | Mm.25794 | 0 | 94.37148218 | 98.88682746 |
| (SEQ ID | | | | | |
| NO: 2273) | | | | | |
| WAN013I4Q_at | 74.39252336 | Mm.210745 | 1E−135 | 88.11881188 | 75.51401869 |
| (SEQ ID | | | | | |
| NO: 2234) | | | | | |
| WAN008EMJ_at | 37.4501992 | Mm.234129 | 3E−58 | 90.37433155 | 37.25099602 |
| (SEQ ID | | | | | |
| NO: 1721) | | | | | |
| U73375_at | 38.81118881 | Mm.3152 | 2E−84 | 85.48812665 | 66.25874126 |
| (SEQ ID | | | | | |
| NO: 2274) | | | | | |
| WAN013I2D_at | 56.75 | Mm.43152 | 2E−86 | 93.24324324 | 55.5 |
| (SEQ ID | | | | | |
| NO: 2275) | | | | | |
| WAN0088ZJ_at | 100 | Mm.4580 | 0 | 93.79699248 | 99.62546816 |
| (SEQ ID | | | | | |
| NO: 2075) | | | | | |

TABLE 30
LLP2 + 4 Test-specific 1.2F DOWN (39genes)

| | | | Human | | |
|---|---|---|---|---|---|
| Qualifier List | Symbol | Title | Unigene ID | eValue | % ID |
| WAN013I6P_f_at | Abcb1a | ATP-binding cassette, sub-family B | #N/A | 6E−40 | 90.90909091 |
| (SEQ ID NO: 1658) | | (MDR/TAP), member 1A | | | |
| WAN008CRT_at | ALG14 | Asparagine-linked glycosylation 14 | Hs.408927 | 4E−47 | 88.39779006 |
| (SEQ ID NO: 1954) | | homolog (yeast) | | | |
| M80243-rc_at | BIRC5 | Baculoviral IAP repeat-containing 5 | Hs.514527 | 4E−38 | 92.37288136 |
| (SEQ ID NO: 1943) | | (survivin) | | | |
| WAN0088K1_at | BRD2 | Bromodomain containing 2 | Hs.75243 | 1E−163 | 88.93129771 |
| (SEQ ID NO: 2276) | | | | | |
| WAN013I8J_at | CCNB2 | Cyclin B2 | Hs.194698 | 1E−173 | 86.92579505 |
| (SEQ ID NO: 2001) | | | | | |
| WAN008CVX_at | CDC20 | CDC20 cell division cycle 20 | Hs.524947 | 1E−169 | 90.66390041 |
| (SEQ ID NO: 1958) | | homolog (S. cerevisiae) | | | |
| WAN008DGK_at | CHAF1A | Chromatin assembly factor 1, | Hs.79018 | 1E−83 | 91.32231405 |
| (SEQ ID NO: 1967) | | subunit A (p150) | | | |
| WAN0088OE_at | Crk | V-crk sarcoma virus CT10 | Hs.638121 | 1E−161 | 94.35483871 |
| (SEQ ID NO: 2176) | | oncogene homolog (avian) | | | |
| WAN008E4R_at | DHX16 | DEAH (Asp-Glu-Ala-His) box | Hs.485060 | 4E−84 | 91.63179916 |
| (SEQ ID NO: 2277) | | polypeptide 16 | | | |
| WAN008DYW_at | FARSLA | Phenylalanine-tRNA synthetase- | Hs.23111 | 1E−126 | 86.45418327 |
| (SEQ ID NO: 2278) | | like, alpha subunit | | | |
| WAN008E40_at | FEN1 | Flap structure-specific | Hs.409065 | 1E−121 | 91.22807018 |
| (SEQ ID NO: 2279) | | endonuclease 1 | | | |
| WAN013HYG_x_at | Grb2 | Growth factor receptor bound | Hs.444356 | 4E−47 | 80.13937282 |
| (SEQ ID NO: 2280) | | protein 2 | | | |
| WAN008EVU_f_at | HNRPK | Heterogeneous nuclear | Hs.522257 | 1E−128 | 92.94478528 |
| (SEQ ID NO: 1887) | | ribonucleoprotein K | | | |
| WAN008EX2_x_at | Ifrd1 | Interferon-related developmental | Hs.7879 | 7E−39 | 90.29850746 |
| (SEQ ID NO: 1575) | | regulator 1 | | | |
| WAN008E3O_at | LINCR | Likely ortholog of mouse lung- | Hs.149219 | 3E−19 | 84.61538462 |
| (SEQ ID NO: 1973) | | inducible Neutralized-related | | | |
| | | C3HC4 RING domain protein | | | |
| WAN008CVC_at | Mm.387215 | CDNA, clone:Y0G0110B16, | #N/A | 1E−119 | 97.95081967 |
| (SEQ ID NO: 2281) | | strand:plus, | | | |
| | | reference:ENSEMBL:Mouse- | | | |
| | | Transcript- | | | |
| | | ENST:ENSMUST00000058619, | | | |
| | | based on BLAT search | | | |
| WAN0088XN_at | MTHFD1L | Methylenetetrahydrofolate | Hs.591343 | 1E−99 | 90.57239057 |
| (SEQ ID NO: 2282) | | dehydrogenase (NADP+ | | | |
| | | dependent) 1-like | | | |
| WAN008EV8_at | MTHFD2 | Methylenetetrahydrofolate | Hs.469030 | 7E−50 | 88.35978836 |
| (SEQ ID NO: 2189) | | dehydrogenase (NADP+ | | | |
| | | dependent) 2, | | | |
| | | methenyltetrahydrofolate | | | |
| | | cyclohydrolase | | | |
| WAN008EOB_at | NOL1 | Nucleolar protein 1, 120 kDa | Hs.534334 | 8E−48 | 89.80582524 |
| (SEQ ID NO: 1629) | | | | | |
| WAN008CTA_at | NOLC1 | Nucleolar and coiled-body | Hs.523238 | 1E−101 | 89.12386707 |
| (SEQ ID NO: 1957) | | phosphoprotein 1 | | | |
| WAN013I8D_at | PARP1 | Poly (ADP-ribose) polymerase | Hs.177766 | 2E−43 | 85.57692308 |
| (SEQ ID NO: 2000) | | family, member 1 | | | |
| WAN008DUC_at | PHF14 | PHD finger protein 14 | Hs.159918 | 8E−86 | 94.63414634 |
| (SEQ ID NO: 1795) | | | | | |
| WAN008EBY_at | PLCG1 | Phospholipase C, gamma 1 | Hs.268177 | 1E−175 | 89.63636364 |
| (SEQ ID NO: 2283) | | | | | |
| WAN008EHF_at | POFUT2 | Protein O-fucosyltransferase 2 | Hs.592164 | 5E−77 | 85.84070796 |
| (SEQ ID NO: 2284) | | | | | |
| WAN013HY0_at | PRPF19 | PRP19/PSO4 pre-mRNA | Hs.502705 | 0 | 89.25318761 |
| (SEQ ID NO: 2173) | | processing factor 19 homolog (S. cerevisiae) | | | |
| WAN008DQG_at | PRPF3 | PRP3 pre-mRNA processing factor | Hs.11776 | 1E−114 | 92.45901639 |
| (SEQ ID NO: 2285) | | 3 homolog (S. cerevisiae) | | | |
| WAN013I0A_x_at | PRPF38B | PRP38 pre-mRNA processing | Hs.342307 | 4E−84 | 83.39694656 |
| (SEQ ID NO: 2113) | | factor 38 (yeast) domain containing B | | | |
| WAN008E7N_at | RBBP4 | Retinoblastoma binding protein 4 | Hs.16003 | 1E−88 | 89.51048951 |
| (SEQ ID NO: 2286) | | | | | |
| WAN008EQE_x_at | RNPEP | Arginyl aminopeptidase | Hs.497391 | 6E−22 | 85.38461538 |
| (SEQ ID NO: 2287) | | (aminopeptidase B) | | | |
| WAN0088NR_at | SF3B3 | Splicing factor 3b, subunit 3, | Hs.514435 | 1E−131 | 92.39766082 |
| (SEQ ID NO: 2288) | | 130 kDa | | | |
| WAN008EPC_at | SHMT2 | Serine hydroxymethyltransferase 2 | Hs.75069 | 1E−143 | 90.43062201 |
| (SEQ ID NO: 2289) | | (mitochondrial) | | | |
| WAN008E5L_at | SLC1A5 | Solute carrier family 1 (neutral | Hs.631582 | 8E−42 | 84.16666667 |
| (SEQ ID NO: 1619) | | amino acid transporter), member 5 | | | |
| WAN0088S8_at | SLC29A1 | Solute carrier family 29 (nucleoside | Hs.25450 | 3E−35 | 81.3559322 |
| (SEQ ID NO: 1591) | | transporters), member 1 | | | |
| WAN008DCP_at | TBC1D10A | TBC1 domain family, member 10A | Hs.444950 | 0 | 90.84507042 |
| (SEQ ID NO: 2100) | | | | | |
| WAN0088TW_at | TCEB3 | Transcription elongation factor B | Hs.584806 | 1E−173 | 89.36567164 |
| (SEQ ID NO: 1745) | | (SIII), polypeptide 3 (110 kDa, | | | |
| | | elongin A) | | | |
| WAN008CS0_at | TMEM103 | Transmembrane protein 103 | Hs.311100 | 1E−96 | 84.22131148 |
| (SEQ ID NO: 2290) | | | | | |
| WAN008ES6_at | XAB1 | XPA binding protein 1, GTPase | Hs.18259 | 1E−128 | 88.70588235 |
| (SEQ ID NO: 2271) | | | | | |
| WAN008EGH_at | XPOT | Exportin, tRNA (nuclear export | Hs.85951 | 1E−145 | 87.2659176 |
| (SEQ ID NO: 2291) | | receptor for tRNAs) | | | |
| WAN008906_at | Zfp259 | Zinc finger protein 259 | #N/A | 1E−162 | 90.13539652 |
| (SEQ ID NO: 1833) | | | | | |

| | | Mouse | | | |
|---|---|---|---|---|---|
| Qualifier List | % QC | Unigene ID | eValue | % ID | % QC |
| WAN013I6P_f_at | 80 | Mm.207354 | 1E−51 | 93.29268293 | 99.39393939 |
| (SEQ ID NO: 1658) | | | | | |
| WAN008CRT_at | 32.43727599 | Mm.269881 | 5E−51 | 88.77005348 | 33.5125448 |
| (SEQ ID NO: 1954) | | | | | |
| M80243-rc_at | 20.34482759 | Mm.8552 | 1E−36 | 93.45794393 | 18.44827586 |
| (SEQ ID NO: 1943) | | | | | |
| WAN0088K1_at | 95.09981851 | Mm.3444 | 0 | 90.92558984 | 100 |
| (SEQ ID NO: 2276) | | | | | |
| WAN013I8J_at | 44.39215686 | Mm.22592 | 0 | 90.70945946 | 46.43137255 |
| (SEQ ID NO: 2001) | | | | | |
| WAN008CVX_at | 85.1590106 | Mm.289747 | 0 | 92.30769231 | 87.27915194 |
| (SEQ ID NO: 1958) | | | | | |
| WAN008DGK_at | 57.89473684 | Mm.391010 | 1E−101 | 90.84745763 | 70.57416268 |
| (SEQ ID NO: 1967) | | | | | |
| WAN0088OE_at | 77.01863354 | Mm.280125 | 0 | 97.04301075 | 77.01863354 |
| (SEQ ID NO: 2176) | | | | | |
| WAN008E4R_at | 44.25925926 | Mm.390986 | 3E−80 | 90.83333333 | 44.44444444 |
| (SEQ ID NO: 2277) | | | | | |
| WAN008DYW_at | 90.45045045 | Mm.292517 | 0 | 94.22718808 | 96.75675676 |
| (SEQ ID NO: 2278) | | | | | |
| WAN008E40_at | 61.40035907 | Mm.2952 | 1E−136 | 92.77456647 | 62.11849192 |
| (SEQ ID NO: 2279) | | | | | |
| WAN013HYG_x_at | 86.18618619 | Mm.383426 | 1E−89 | 85.07936508 | 94.59459459 |
| (SEQ ID NO: 2280) | | | | | |
| WAN008EVU_f_at | 97.60479042 | Mm.142872 | 1E−133 | 93.11377246 | 100 |
| (SEQ ID NO: 1887) | | | | | |
| WAN008EX2_x_at | 100 | Mm.168 | 5E−63 | 97.76119403 | 100 |
| (SEQ ID NO: 1575) | | | | | |
| WAN008E3O_at | 38.01169591 | Mm.389110 | 3E−76 | 85.3372434 | 99.70760234 |
| (SEQ ID NO: 1973) | | | | | |
| WAN008CVC_at | 100 | Mm.387215 | 1E−117 | 97.54098361 | 100 |
| (SEQ ID NO: 2281) | | | | | |
| WAN0088XN_at | 54.59558824 | Mm.184752 | 1E−126 | 94.27609428 | 54.59558824 |
| (SEQ ID NO: 2282) | | | | | |
| WAN008EV8_at | 68.23104693 | Mm.443 | 4E−84 | 91.05691057 | 88.80866426 |
| (SEQ ID NO: 2189) | | | | | |
| WAN008EOB_at | 42.4742268 | Mm.29203 | 1E−120 | 87.24832215 | 92.16494845 |
| (SEQ ID NO: 1629) | | | | | |
| WAN008CTA_at | 59.63963964 | Mm.402190 | 3E−28 | 89.90825688 | 19.63963964 |
| (SEQ ID NO: 1957) | | | | | |
| WAN013I8D_at | 35.01683502 | Mm.277779 | 1E−102 | 87.78054863 | 67.50841751 |
| (SEQ ID NO: 2000) | | | | | |
| WAN008DUC_at | 99.51456311 | Mm.212411 | 4E−74 | 92.19512195 | 99.51456311 |
| (SEQ ID NO: 1795) | | | | | |
| WAN008EBY_at | 99.45750452 | Mm.44463 | 0 | 92.58589512 | 100 |
| (SEQ ID NO: 2283) | | | | | |
| WAN008EHF_at | 61.30198915 | Mm.203556 | 1E−179 | 94.58128079 | 73.41772152 |
| (SEQ ID NO: 2284) | | | | | |
| WAN013HY0_at | 100 | Mm.358657 | 0 | 93.26047359 | 100 |
| (SEQ ID NO: 2173) | | | | | |
| WAN008DQG_at | 99.02597403 | Mm.279872 | 1E−121 | 93.18181818 | 100 |
| (SEQ ID NO: 2285) | | | | | |
| WAN013I0A_x_at | 99.05482042 | Mm.51049 | 1E−173 | 89.69465649 | 99.05482042 |
| (SEQ ID NO: 2113) | | | | | |
| WAN008E7N_at | 94.38943894 | Mm.12145 | 1E−113 | 92.07920792 | 100 |
| (SEQ ID NO: 2286) | | | | | |
| WAN008EQE_x_at | 59.09090909 | Mm.291048 | 7E−45 | 85.45454545 | 100 |
| (SEQ ID NO: 2287) | | | | | |
| WAN0088NR_at | 81.04265403 | Mm.236123 | 1E−151 | 94.9704142 | 80.09478673 |
| (SEQ ID NO: 2288) | | | | | |
| WAN008EPC_at | 99.0521327 | Mm.29890 | 1E−142 | 90.68627451 | 96.68246445 |
| (SEQ ID NO: 2289) | | | | | |
| WAN008E5L_at | 45.62737643 | Mm.1056 | 1E−115 | 87.67123288 | 83.26996198 |
| (SEQ ID NO: 1619) | | | | | |
| WAN0088S8_at | 76.12903226 | Mm.29744 | 5E−97 | 86.09756098 | 88.17204301 |
| (SEQ ID NO: 1591) | | | | | |
| WAN008DCP_at | 100 | Mm.28140 | 0 | 95.42253521 | 100 |
| (SEQ ID NO: 2100) | | | | | |
| WAN0088TW_at | 98.52941176 | Mm.27663 | 0 | 93.09701493 | 98.52941176 |
| (SEQ ID NO: 1745) | | | | | |
| WAN008CS0_at | 88.4057971 | Mm.374250 | 1E−152 | 87.96992481 | 96.37681159 |
| (SEQ ID NO: 2290) | | | | | |
| WAN008ES6_at | 98.60788863 | Mm.348649 | 1E−168 | 92.6713948 | 98.14385151 |
| (SEQ ID NO: 2271) | | | | | |
| WAN008EGH_at | 93.84885764 | Mm.25042 | 1E−150 | 87.64044944 | 93.84885764 |
| (SEQ ID NO: 2291) | | | | | |
| WAN008906_at | 94.86238532 | Mm.17519 | 0 | 92.8440367 | 100 |
| (SEQ ID NO: 1833) | | | | | |

Standard cell engineering methods are used to modify target genes to effect desired cell phenotypes. As discussed above, target genes are modified to achieve desired CHO cell phenotypes by interfering RNA, conventional gene knockout or overexpression methods. Typically, knockout methods or stable transfection methods with overexpression constructs are used to engineer modified CHO cell lines. Other suitable methods are discussed in the general description section and known in the art.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

### INCORPORATION BY REFERENCE

All sequence accession numbers, publications and patent documents cited in this application are incorporated by reference in their entirety for all purposes to the same extent as if the contents of each individual publication or patent document was incorporated herein.

What is claimed is:

1. A method for identifying proteins regulating or indicative of a cell culture phenotype in a cell line, the method comprising:
generating a protein expression profile of a sample derived from a test cell line;
comparing the protein expression profile to a control profile derived from a control cell line; and
identifying one or more differentially expressed proteins based on the comparison,
wherein the test cell line has a cell culture phenotype distinct from that of the control cell line, and the one or more differentially expressed proteins are capable of regulating or indicating the cell culture phenotype.

2. The method of claim 1, wherein the cell line is a Chinese hamster ovary (CHO) cell line.

3. The method of claim 1, wherein the cell culture phenotype is a cell growth rate, a cellular productivity, a peak cell density, a sustained cell viability, a rate of ammonia production or consumption, or a rate of lactate production or consumption.

4. The method of claim 3, wherein the cell culture phenotype is a maximum cellular productivity.

5. The method of claim 3, wherein the cell culture phenotype is a sustained cell viability.

6. The method of claim 3, wherein the cell culture phenotype is a peak cell density.

7. The method of claim 3, wherein the cell culture phenotype is a cell growth rate.

8. The method of claim 1, wherein the protein expression profile is generated by fluorescent two-dimensional differential in-gel electrophoresis.

9. (canceled)

10. (canceled)

11. (canceled)

12. (canceled)

13. (canceled)

14. (canceled)

15. A method for improving cellular productivity of a cell line, the method comprising modulating one or more genes or proteins selected from Tables 2, 3, 9, 10, 11, and 12.

16. (canceled)

17. A method for improving cell growth rate of a cell line, the method comprising modulating one or more genes or proteins selected from Tables 4, 5, 6, 13, 14, 27 and 28.

18. (canceled)

19. A method for increasing peak cell density of a cell line, the method comprising modulating one or more genes or proteins selected from Tables 8, 15, 16, and 17.

20. (canceled)

21. A method for increasing sustained cell viability of a cell line, the method comprising modulating one or more genes or proteins selected from Tables 7, 18 and 19.

22. (canceled)

23. A method for improving a cell line, the method comprising modulating one or more genes selected from Tables 20, 24, 25, and 26.

24. A method for modulating a rate of lactate production or consumption in a cell line, the method comprising modulating one or more genes selected from Tables 29 and 30.

25. A method for improving a cell line, the method comprising up-regulating or down-regulating at least two genes or proteins, wherein a first gene or protein affects a first cell culture phenotype and a second gene or protein affects a second, different cell culture phenotype, wherein the cell culture phenotypes are selected from the group consisting of a cell growth rate, a cellular productivity, a peak cell density, a sustained cell viability, a rate of ammonia production or consumption, or a rate of lactate production or consumption.

26. The method of claim 25, further comprising up-regulating or down-regulating a third gene or protein affecting a third cell culture phenotype different from the first and second cell culture phenotypes.

27. (canceled)

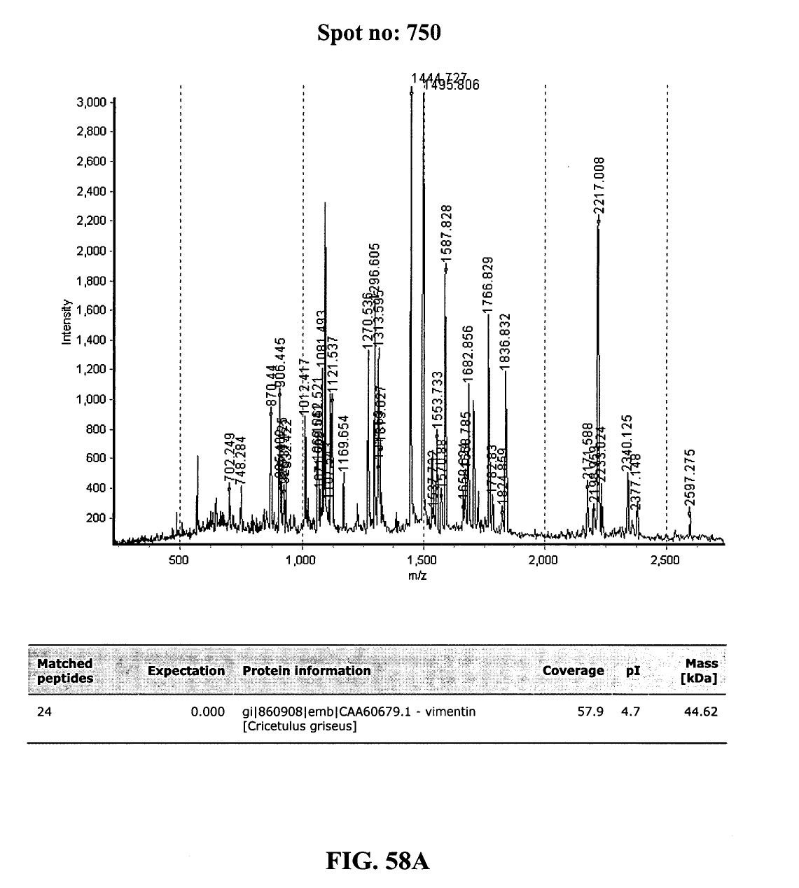

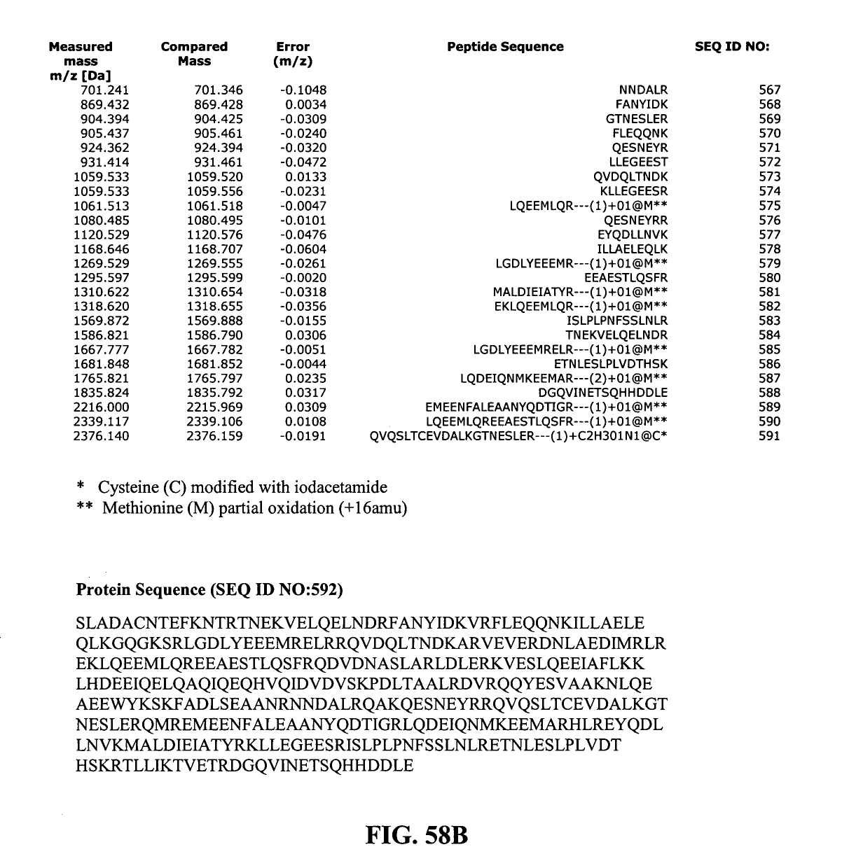

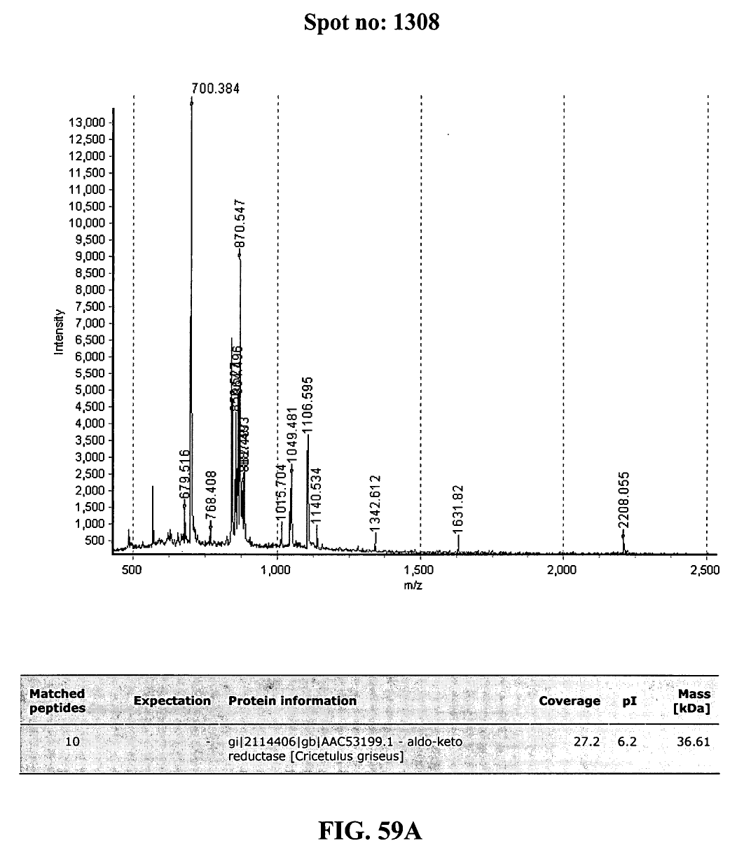

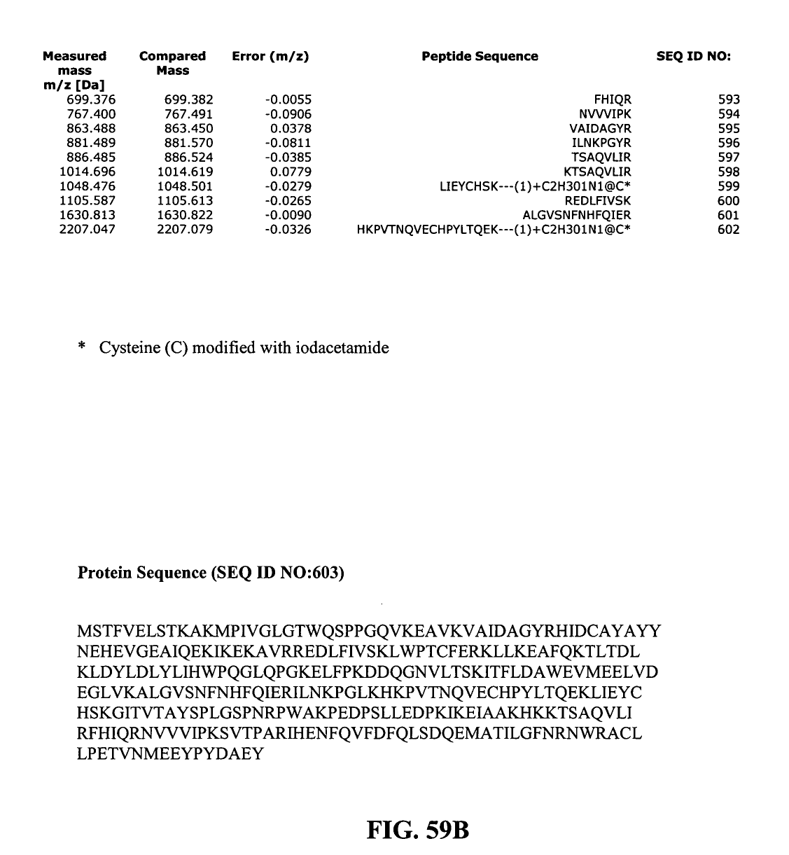

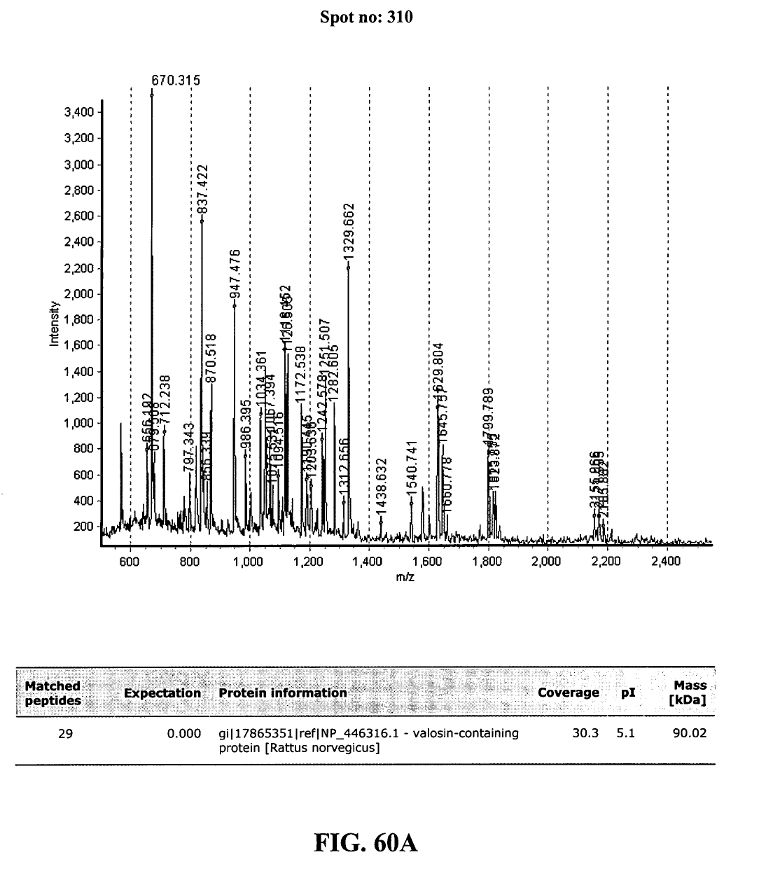

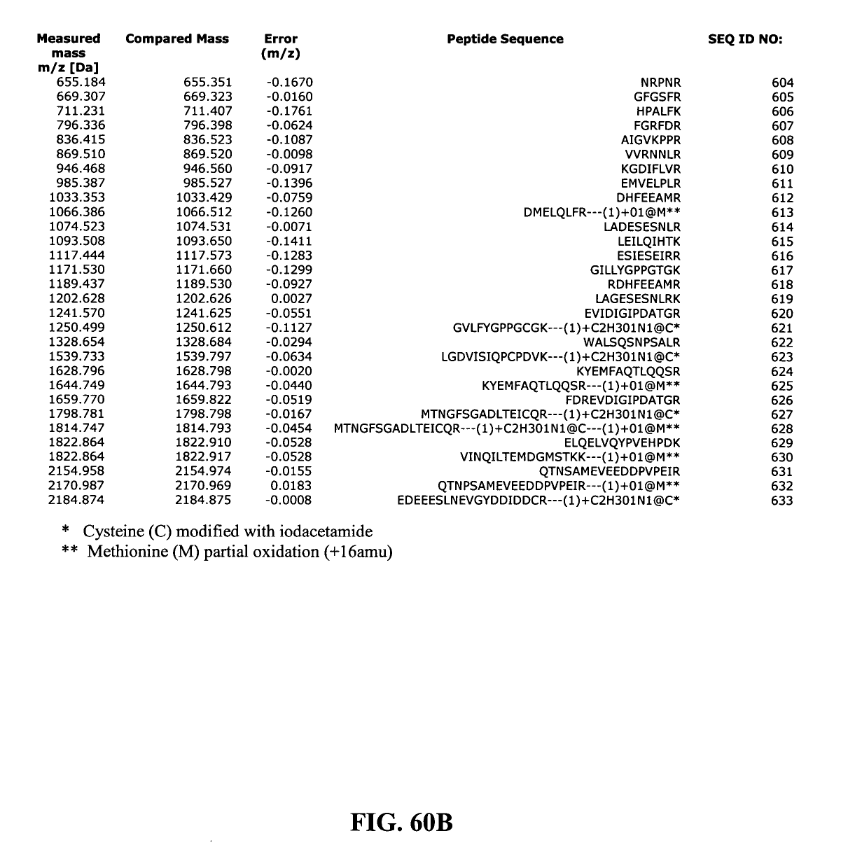

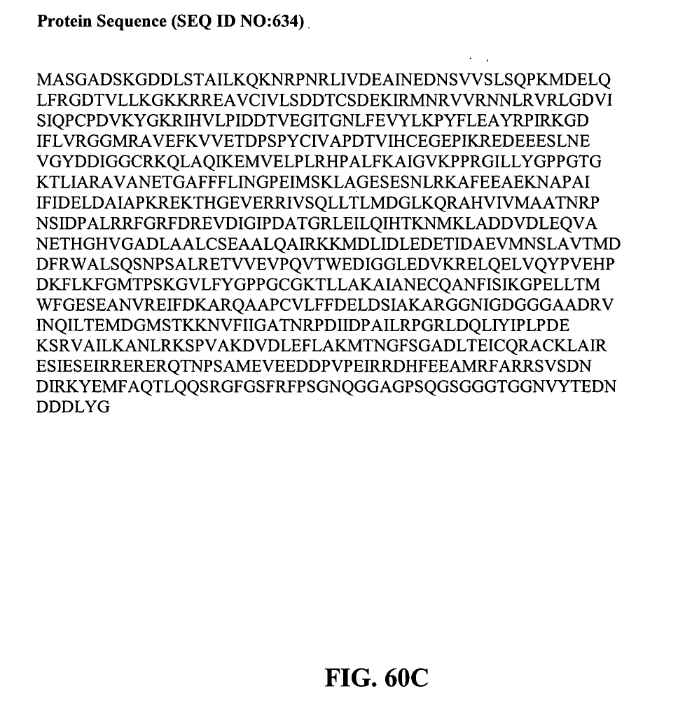

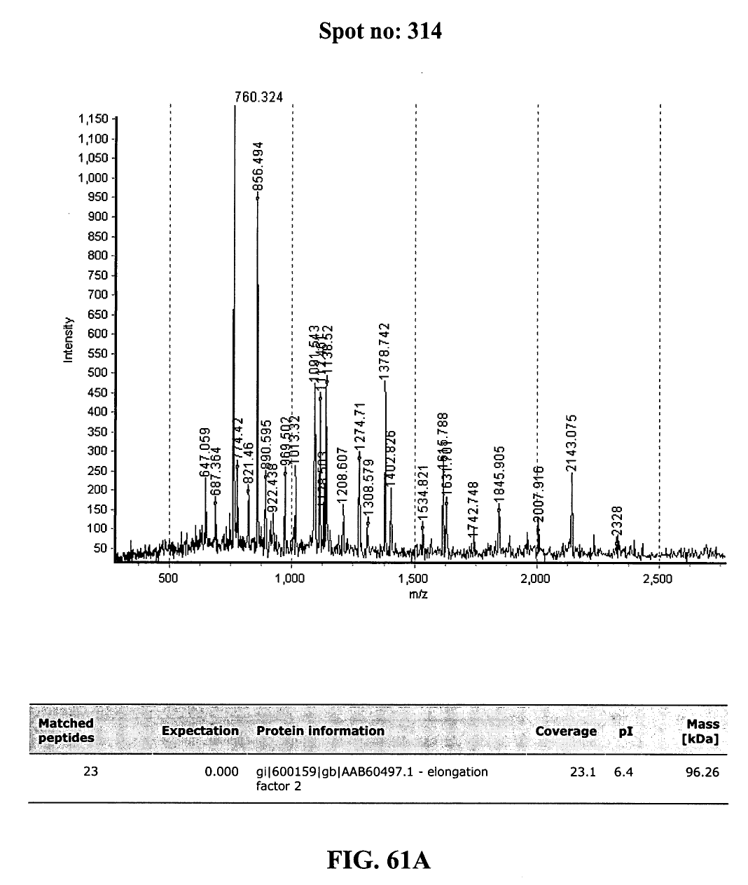

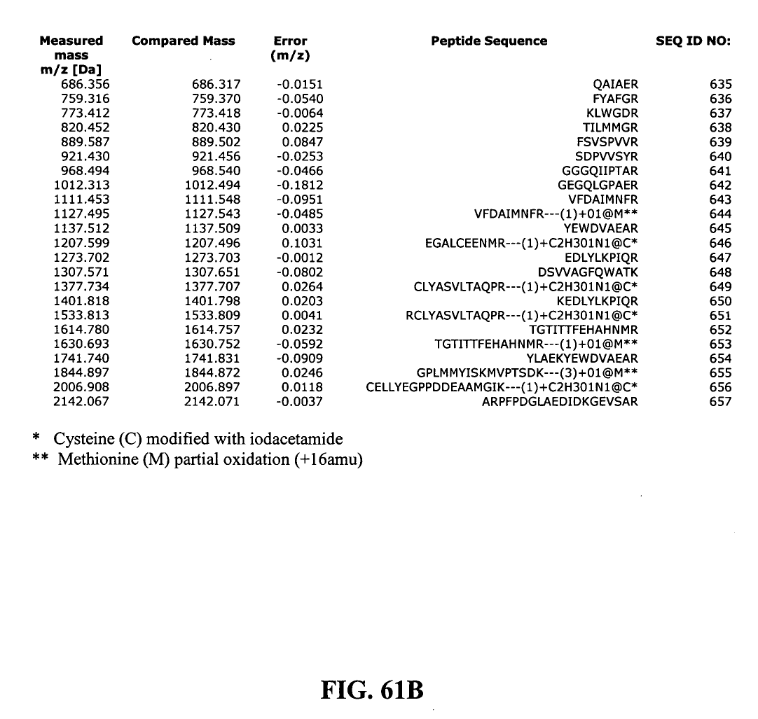

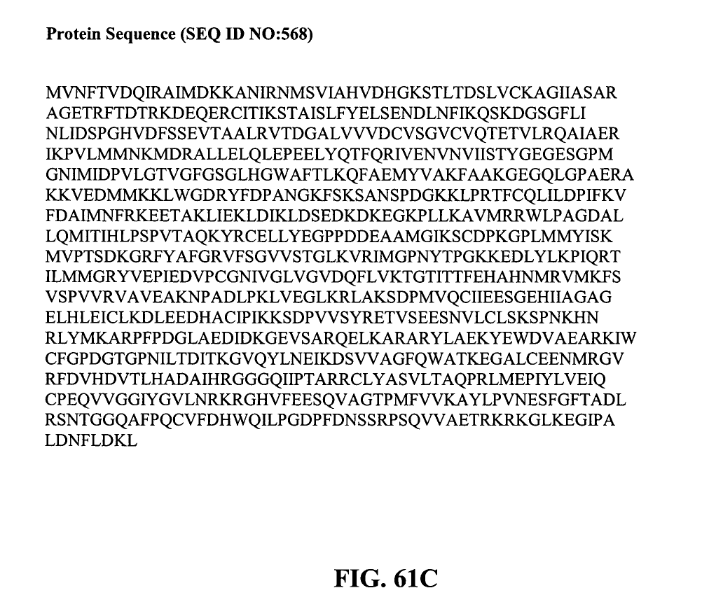

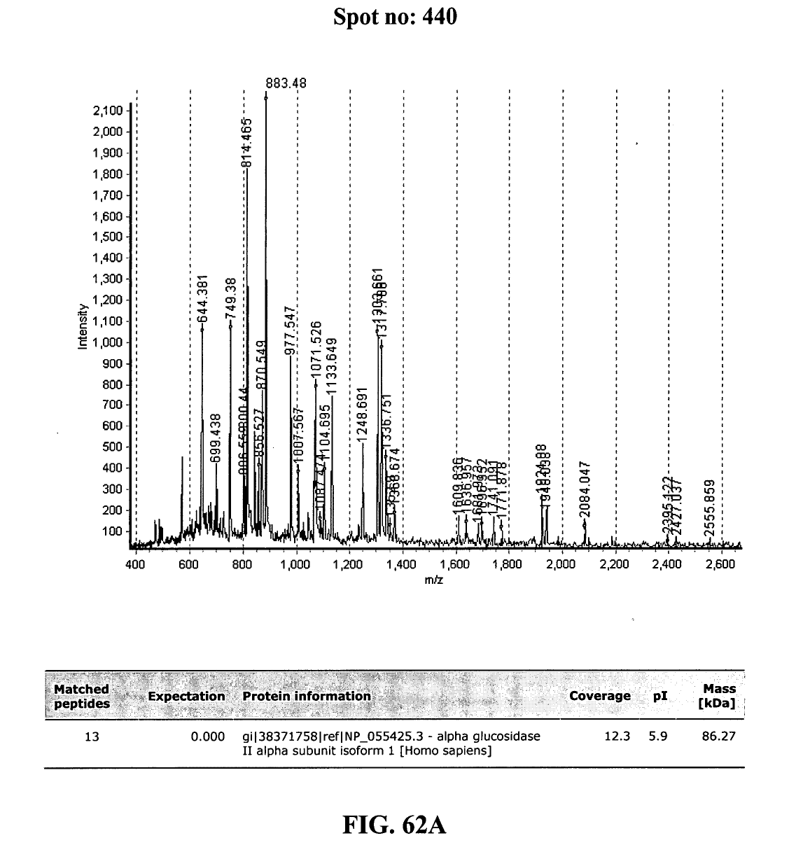

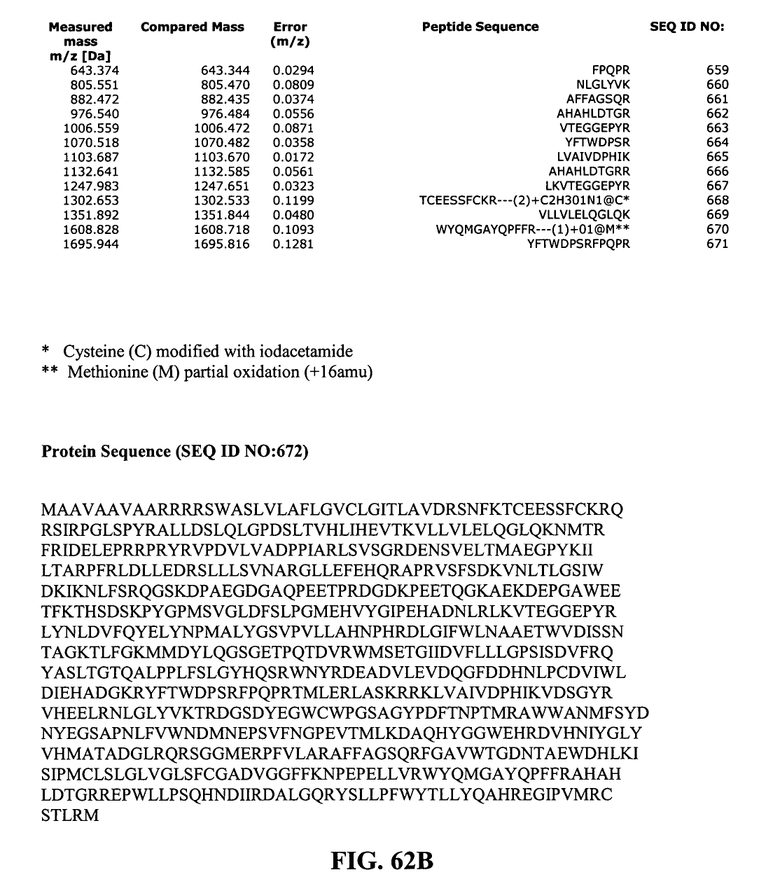

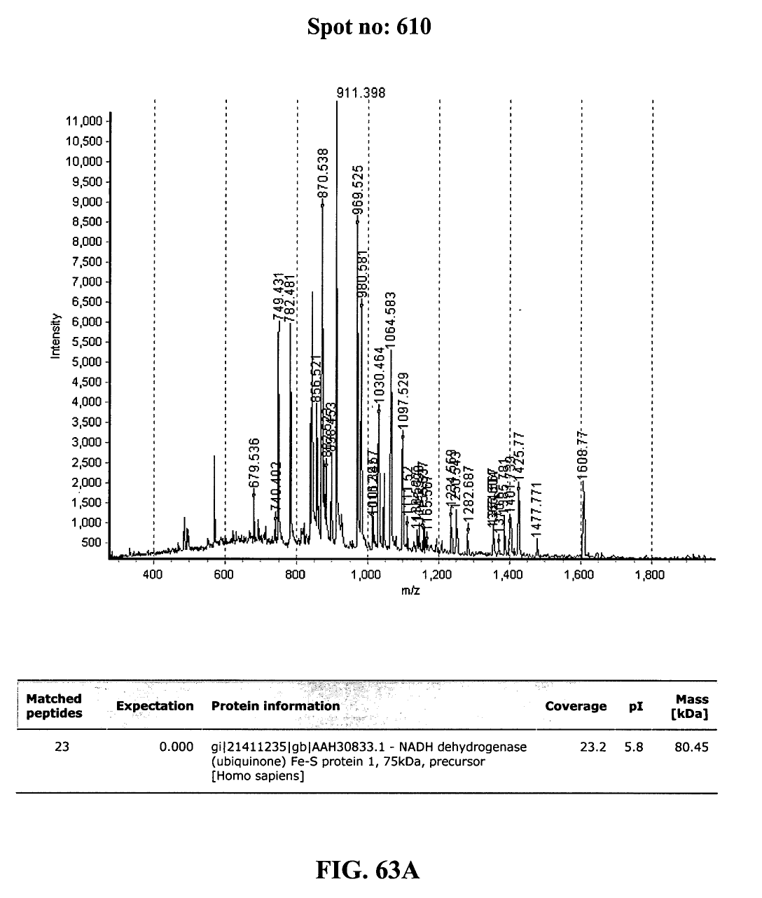

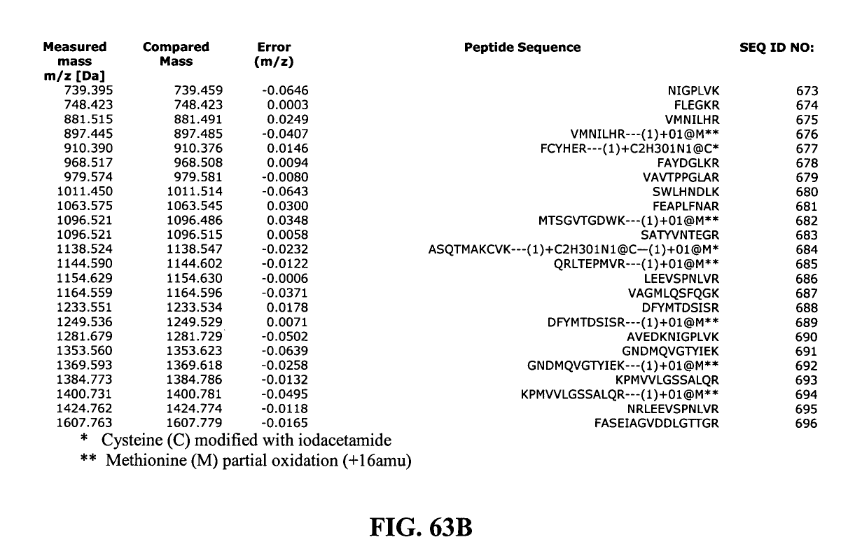

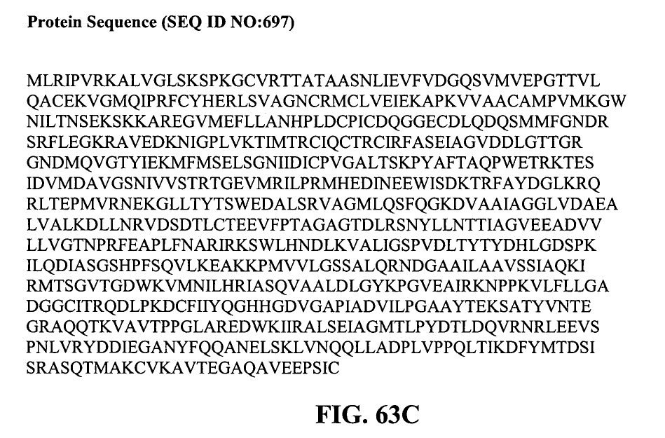

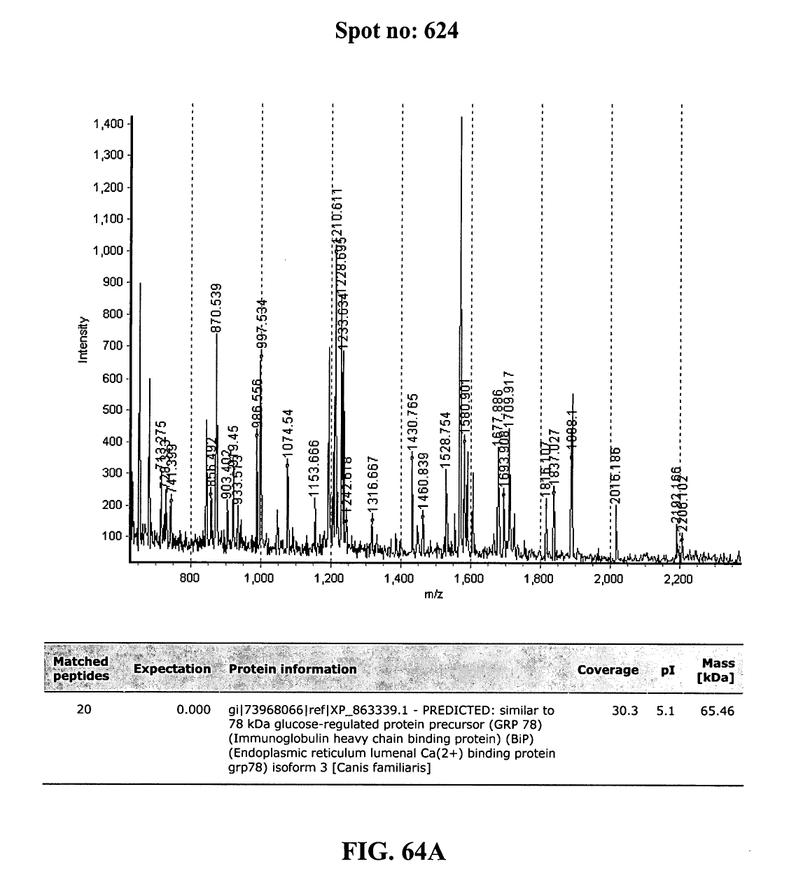

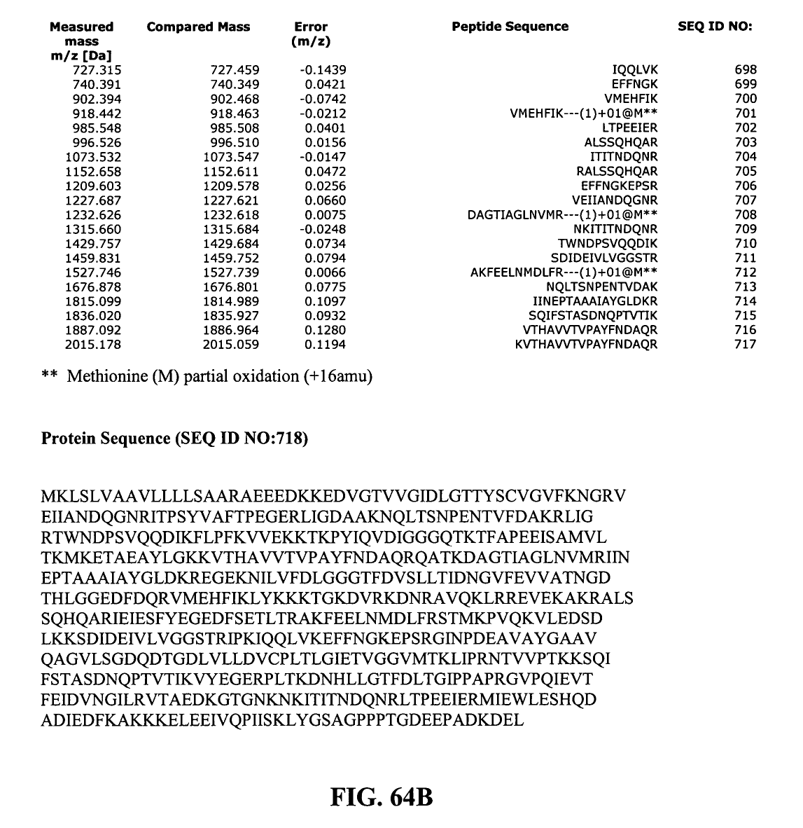

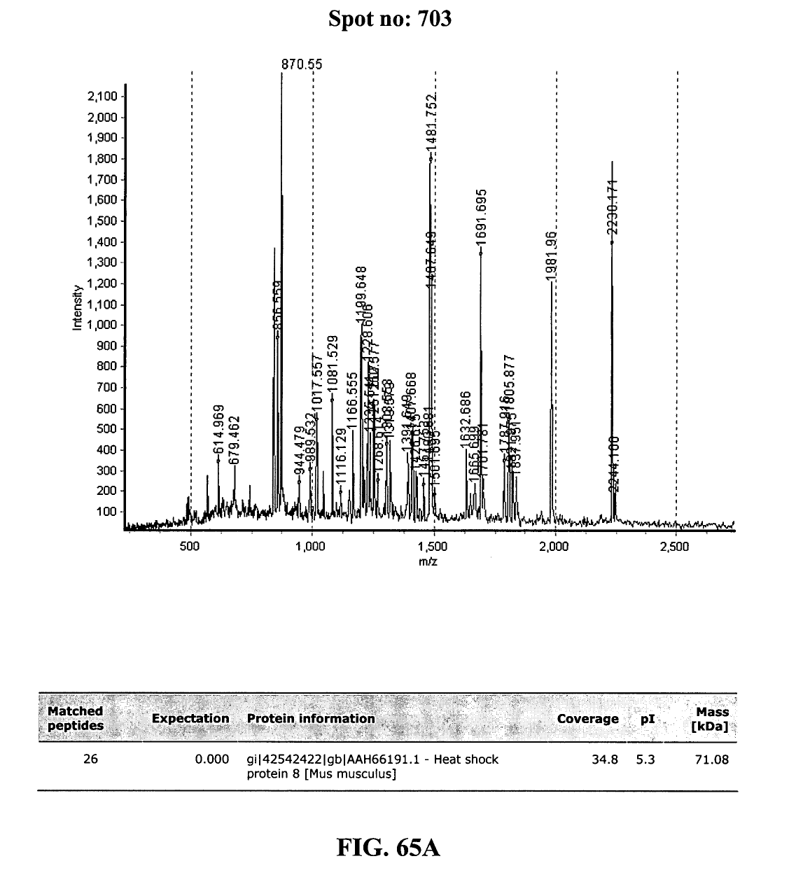

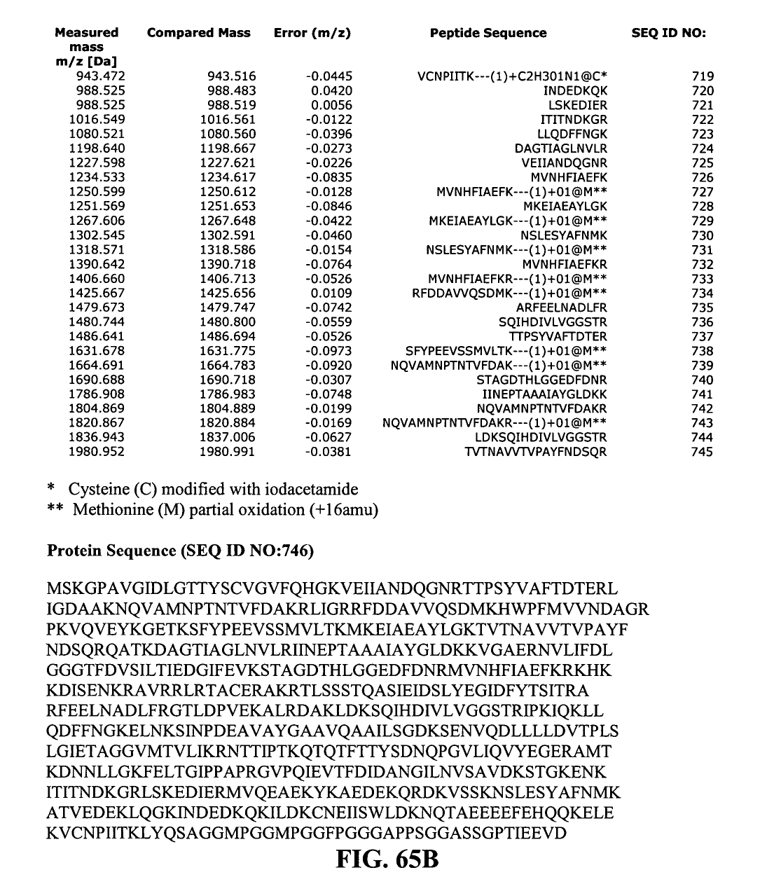

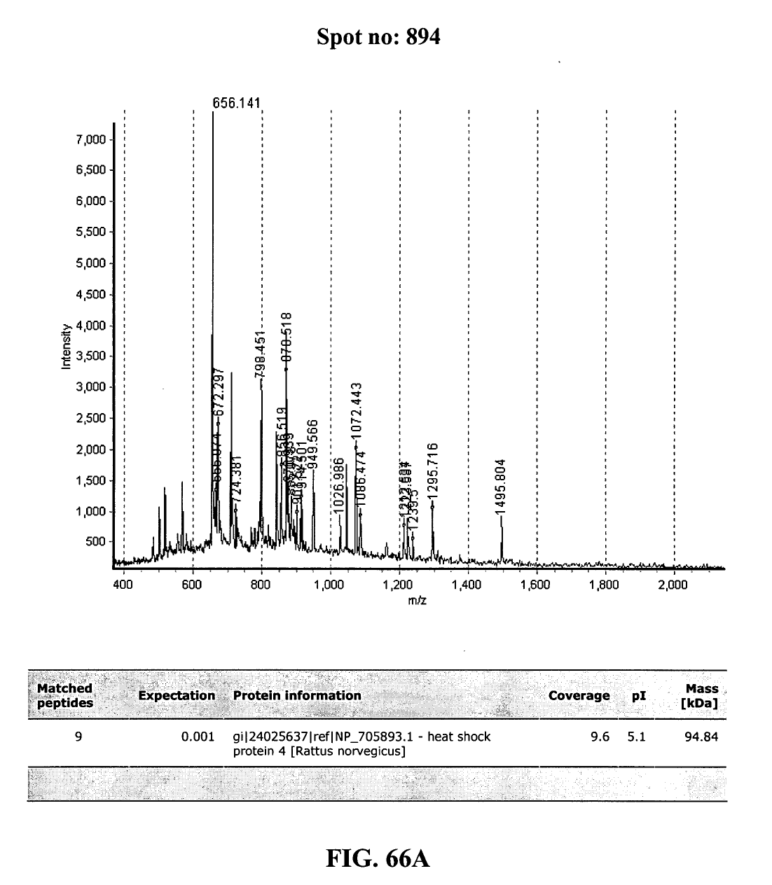

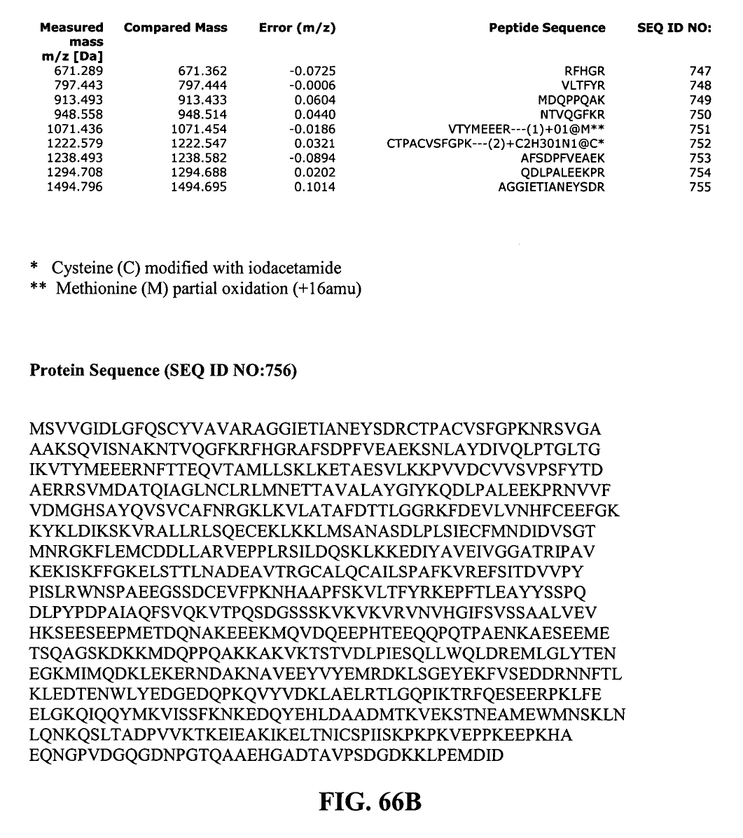

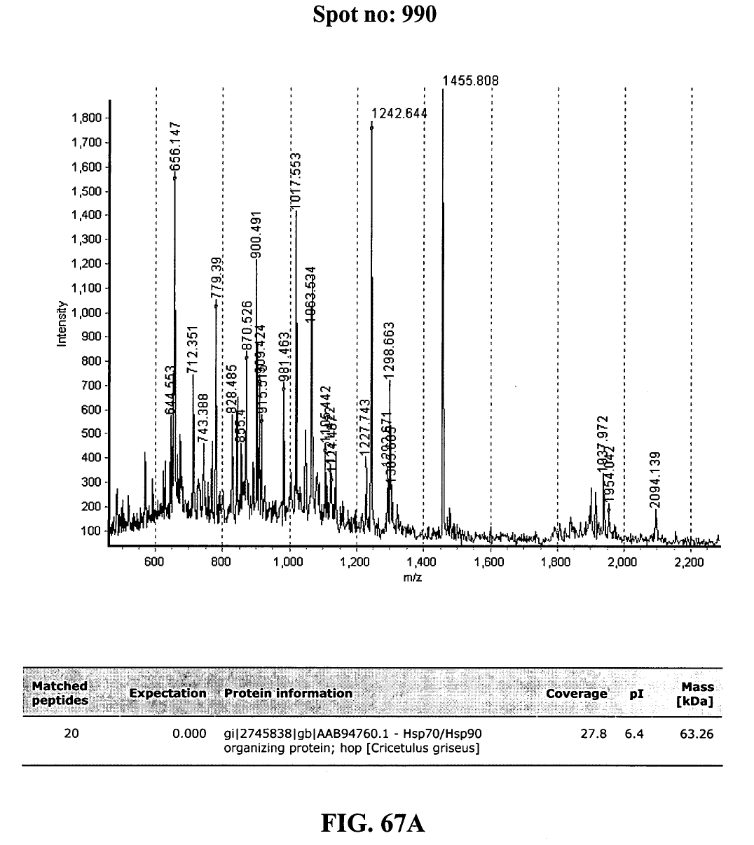

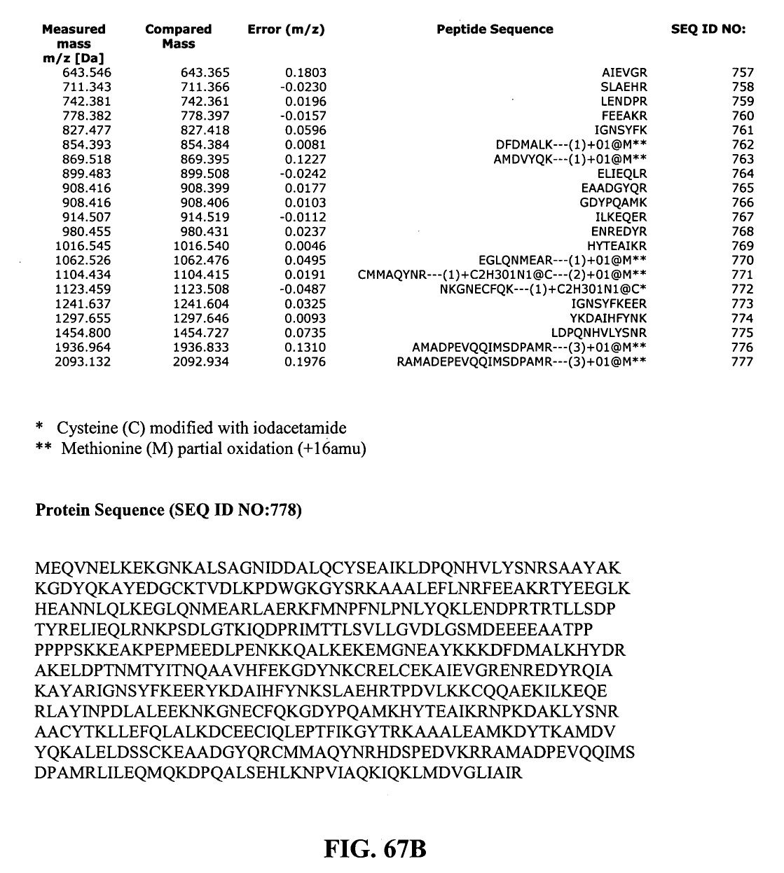

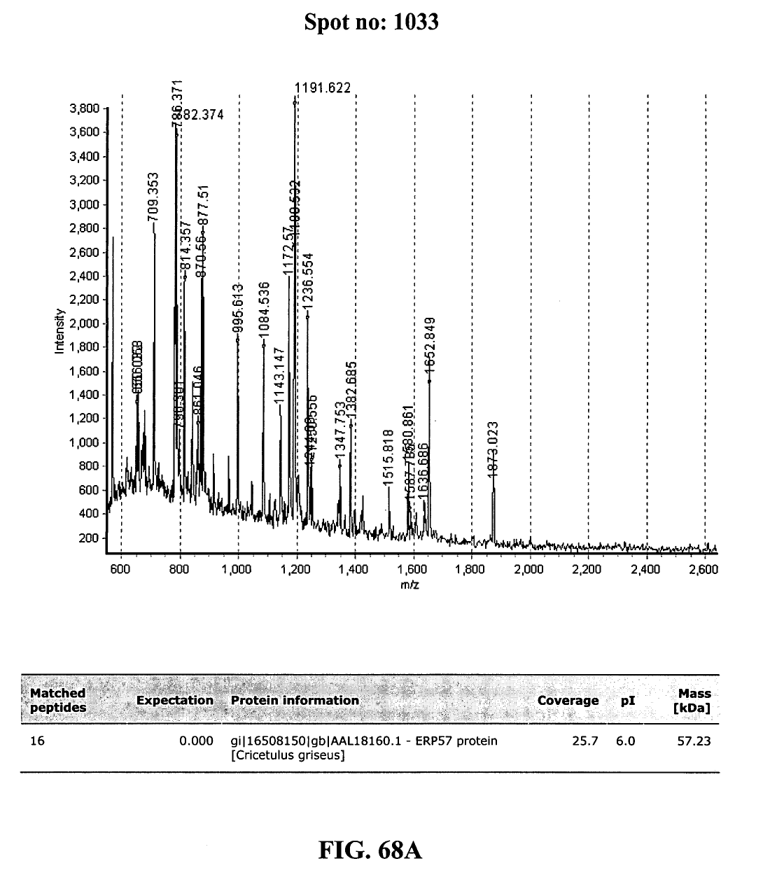

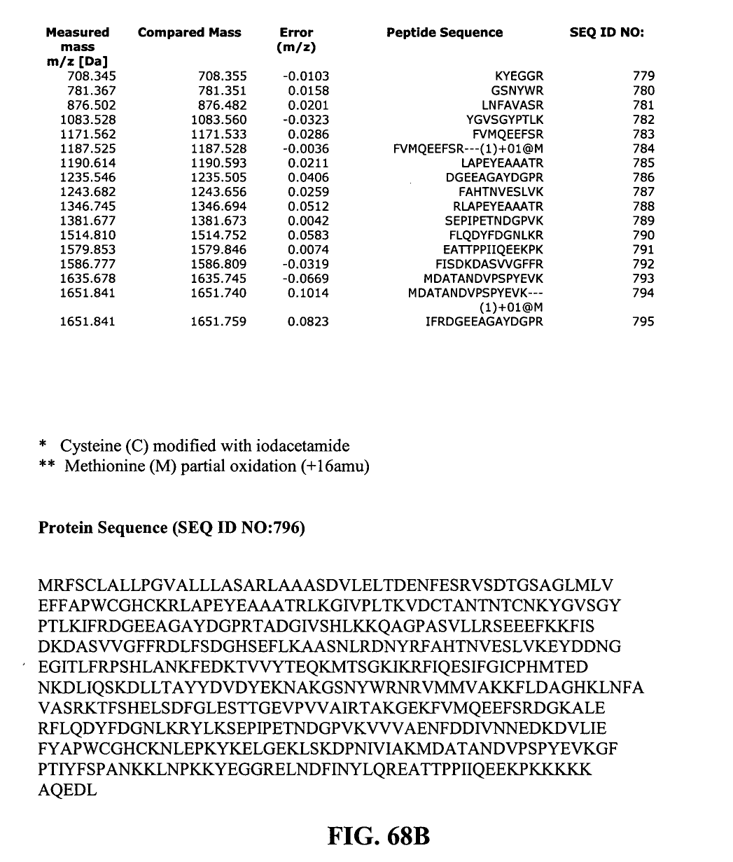

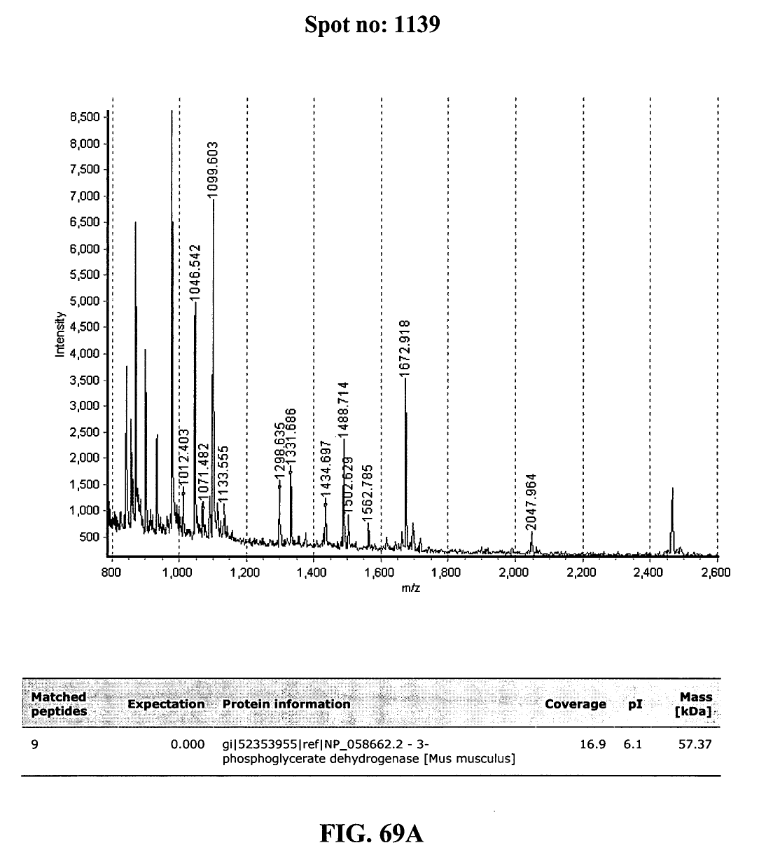

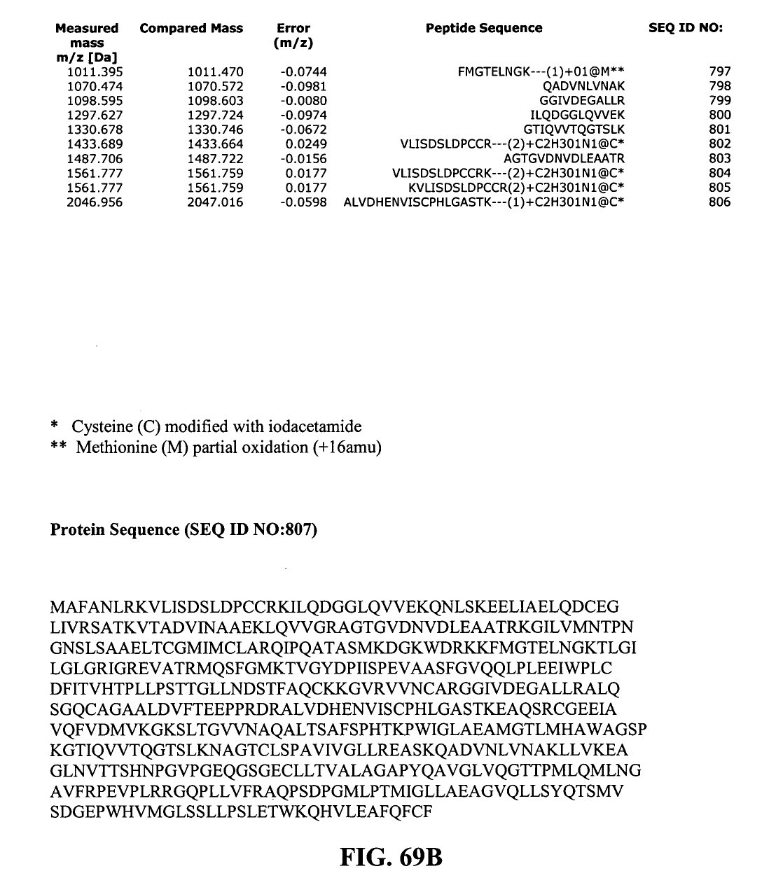

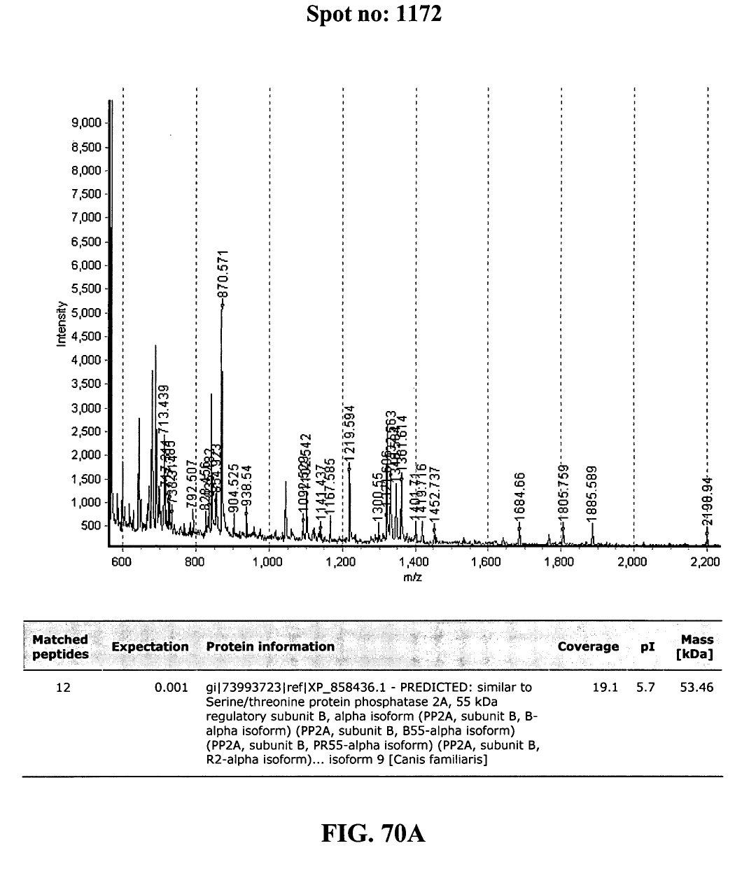

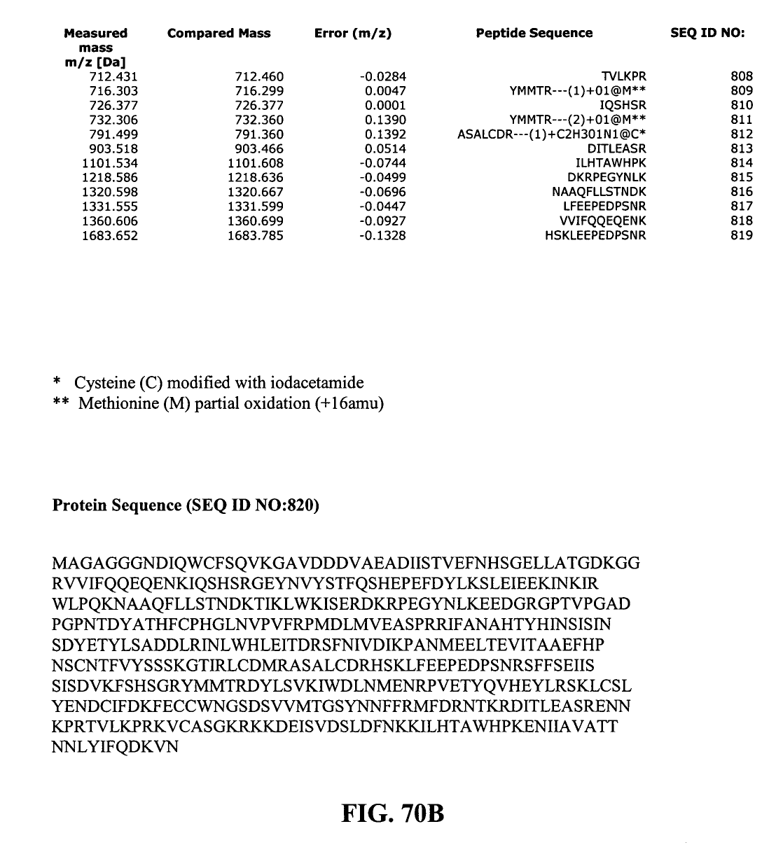

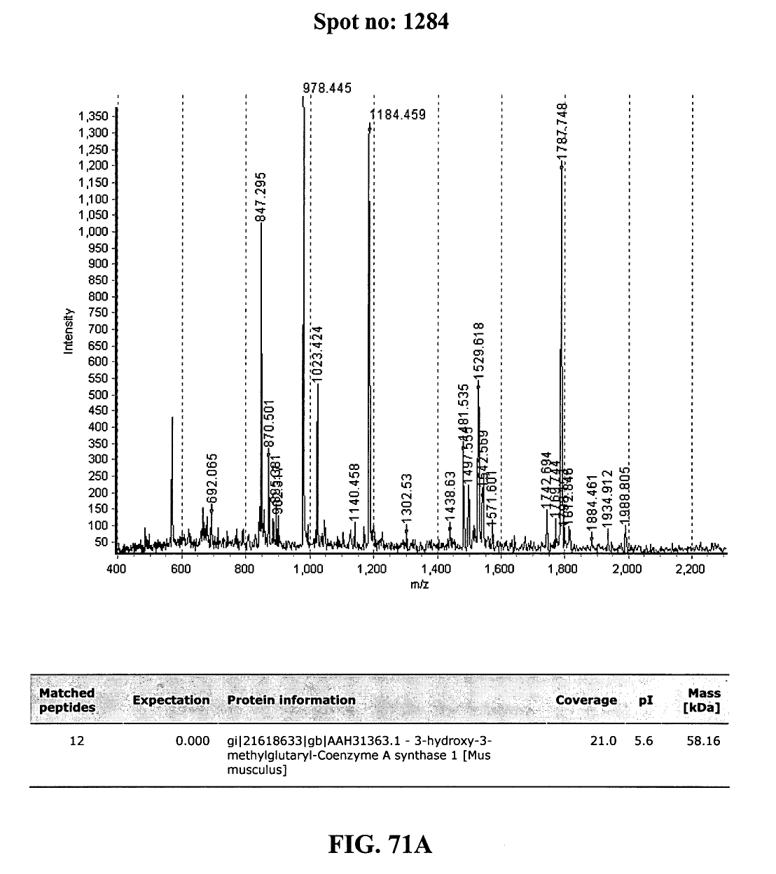

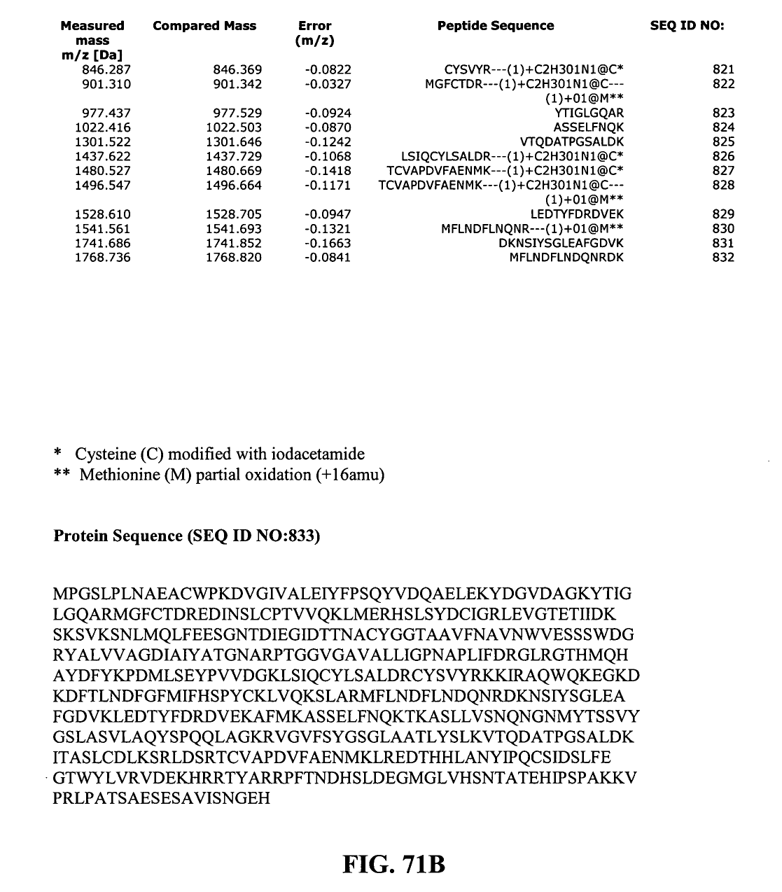

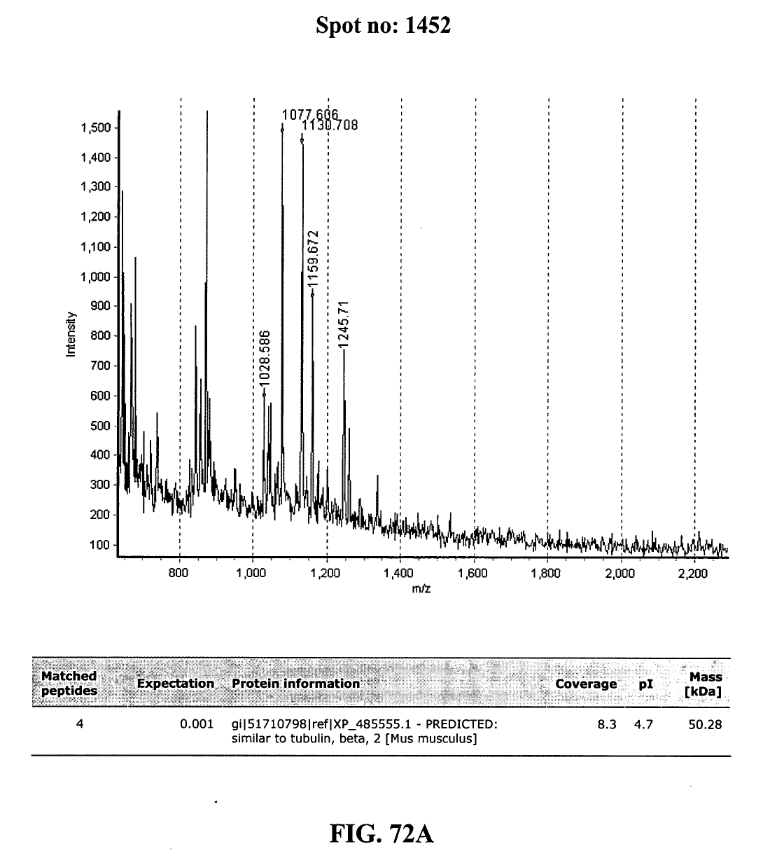

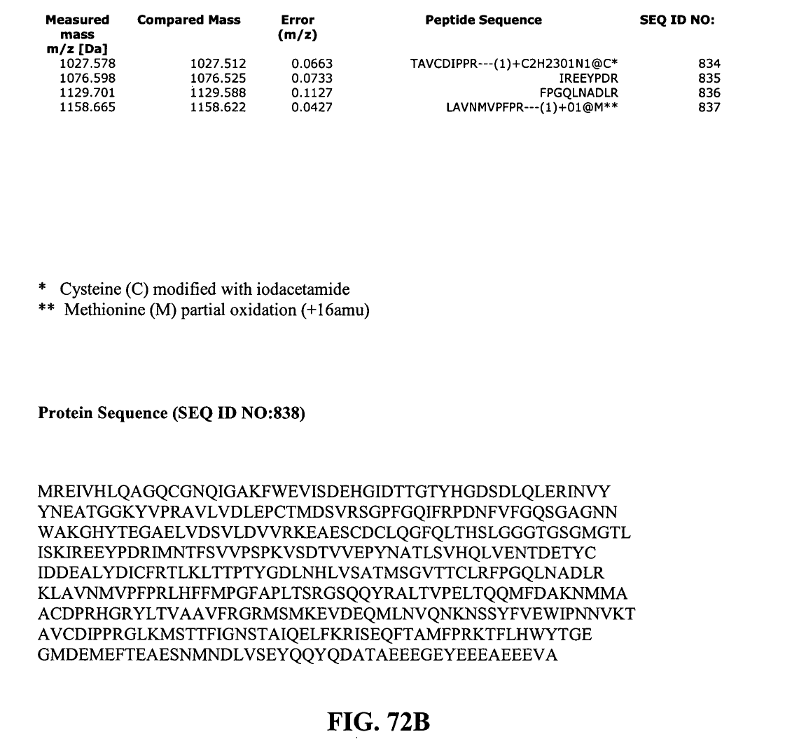

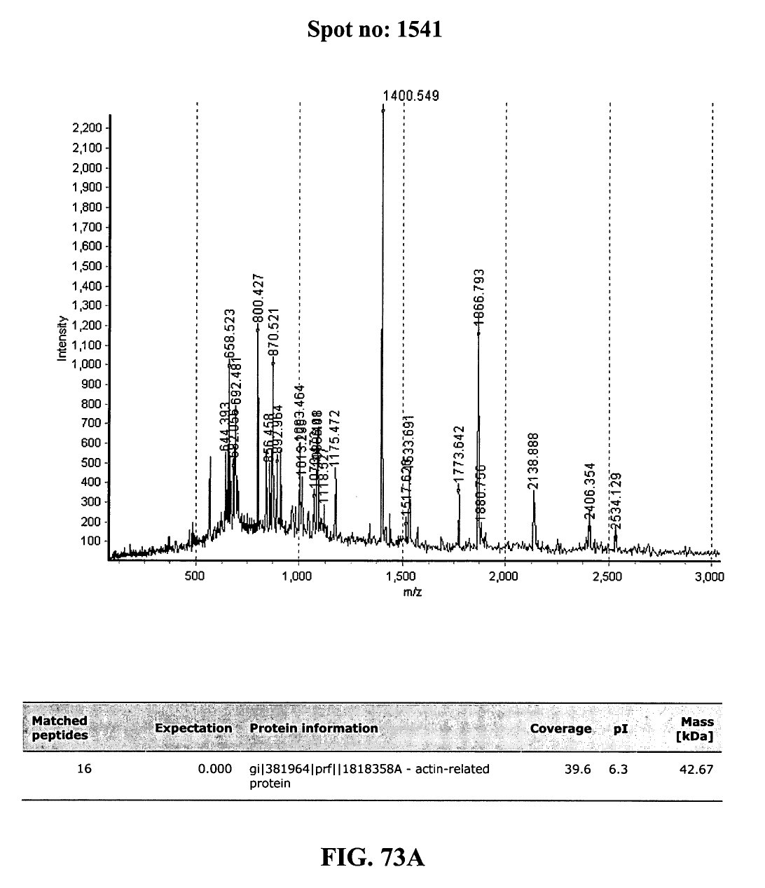

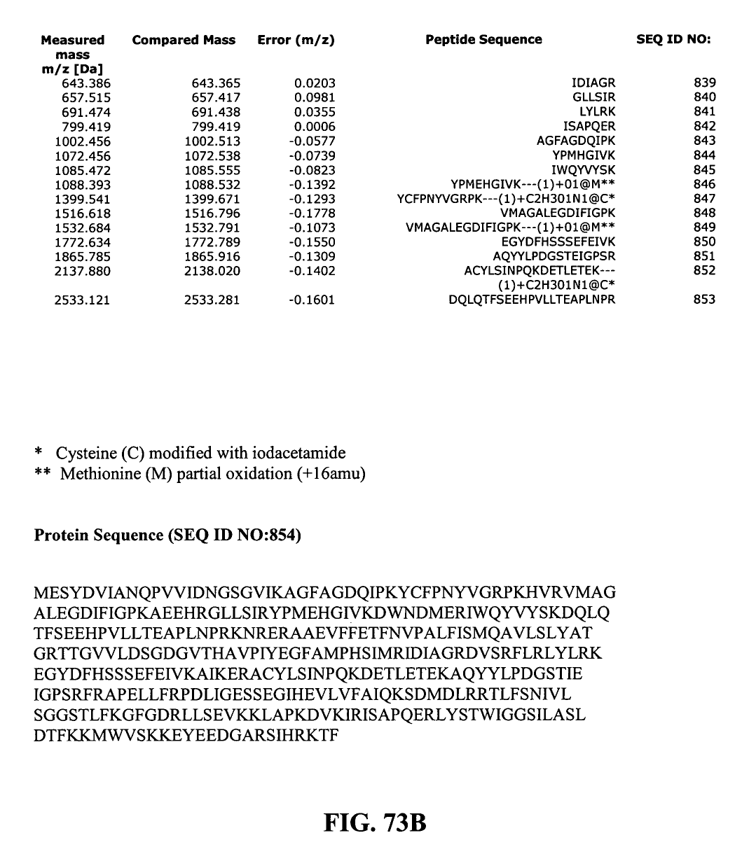

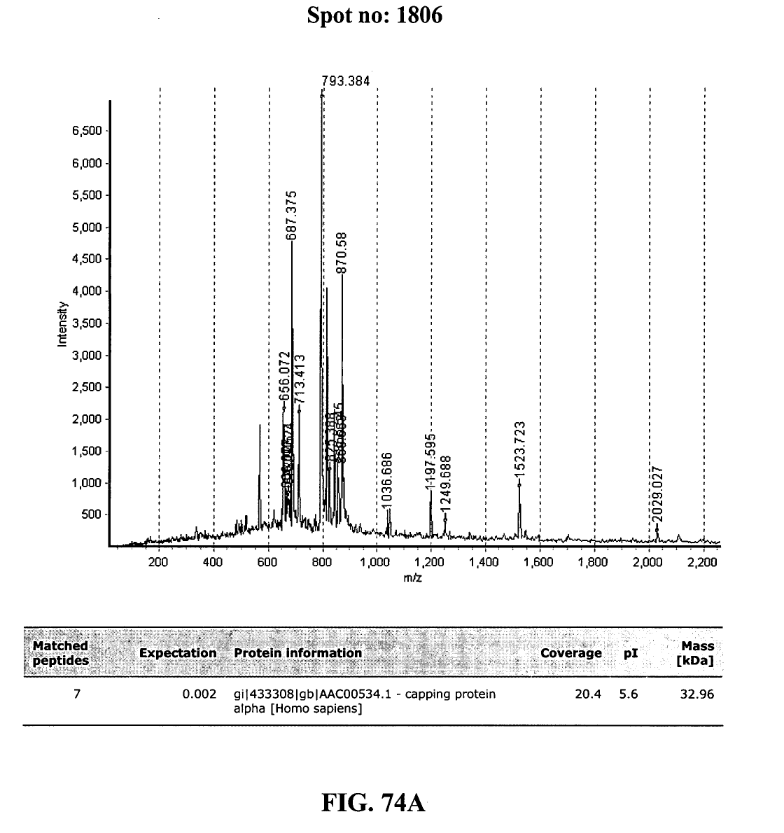

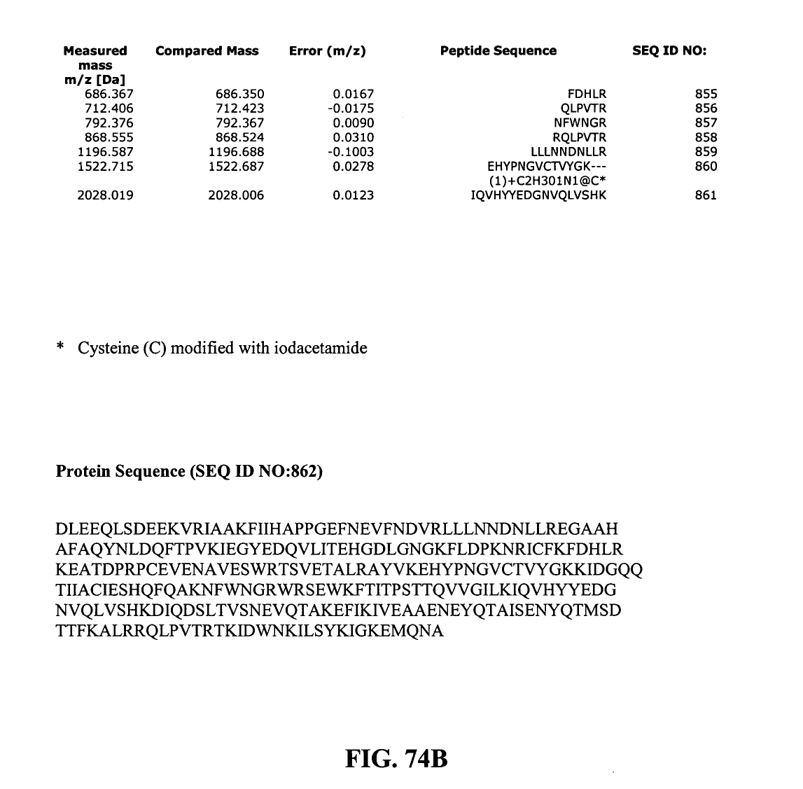

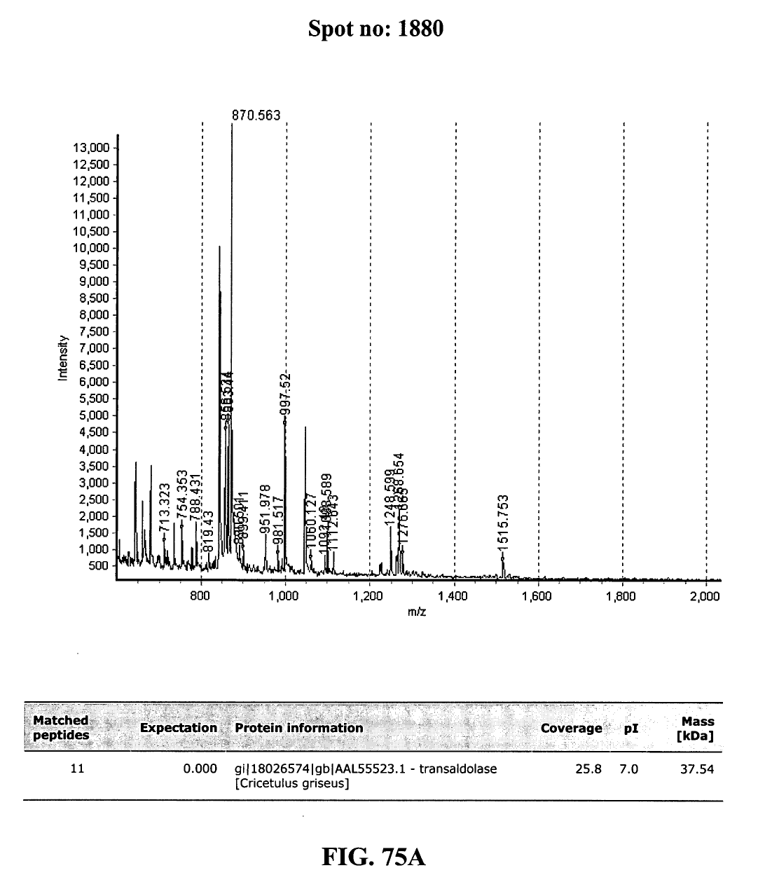

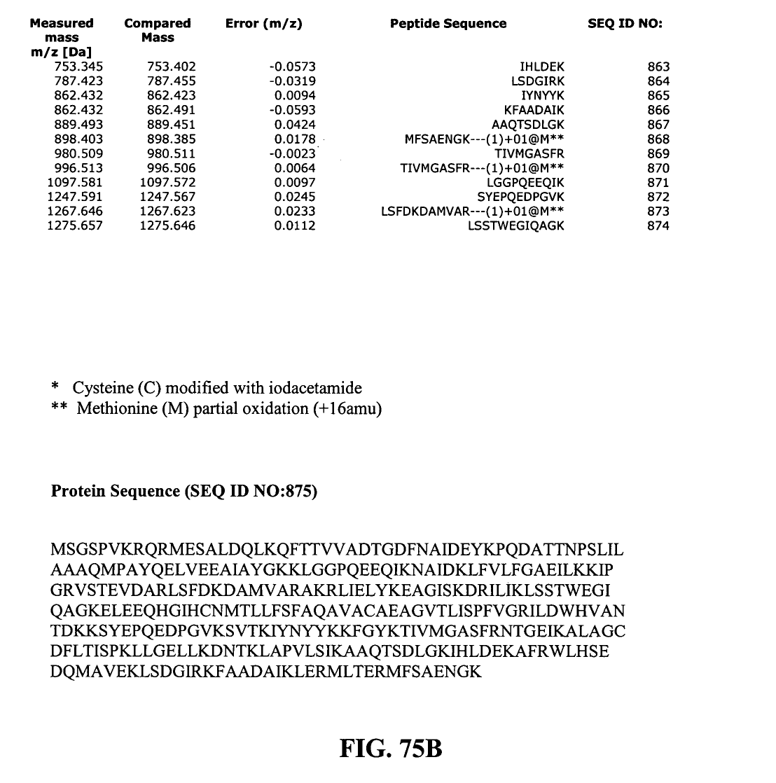

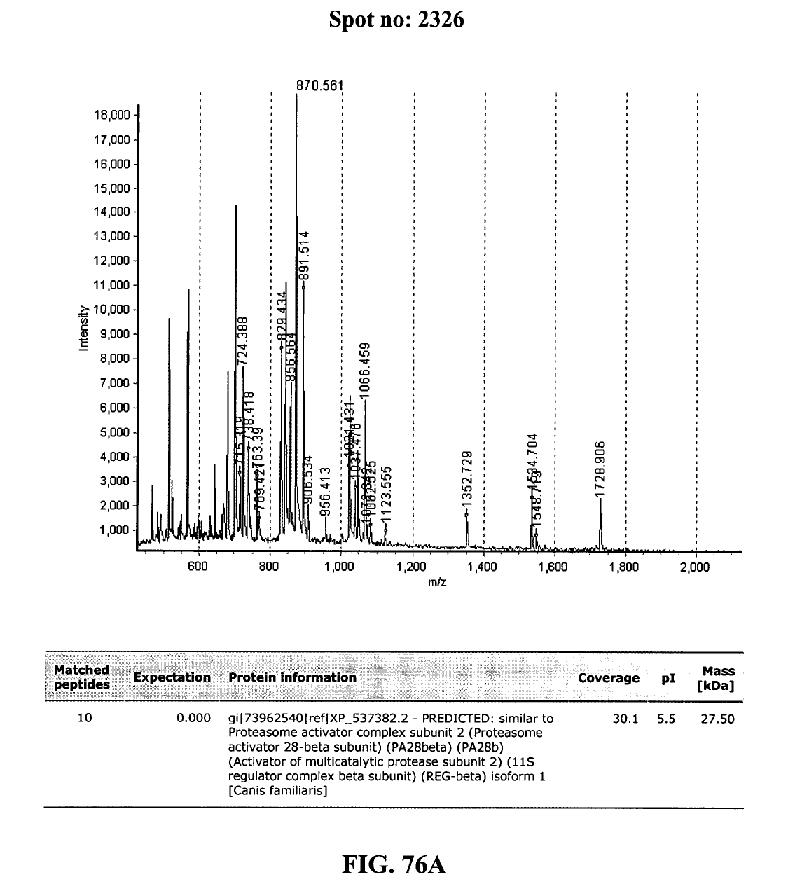

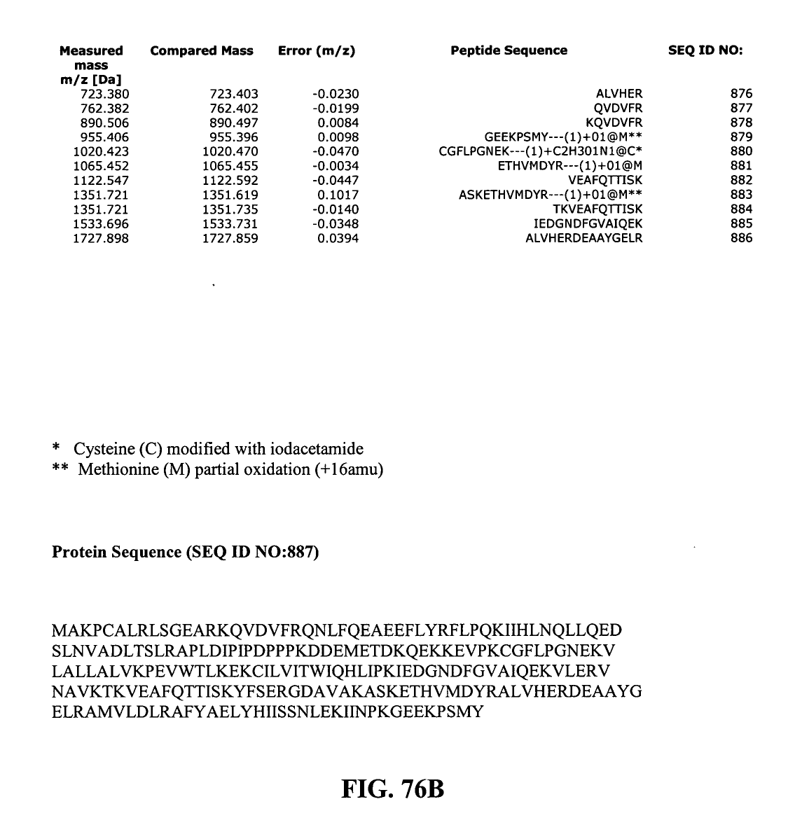

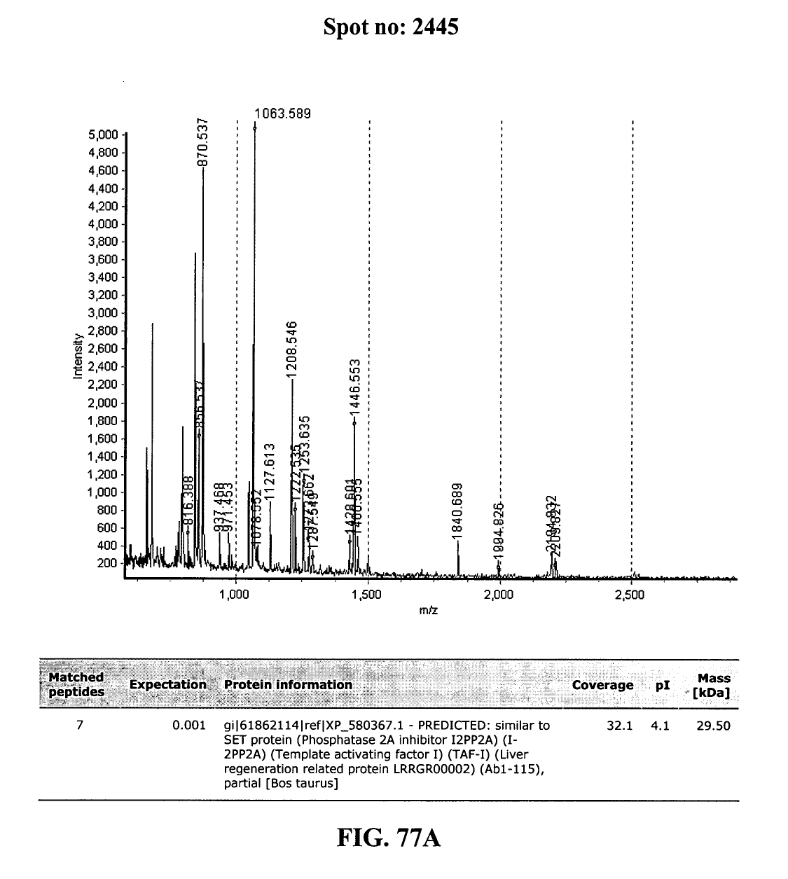

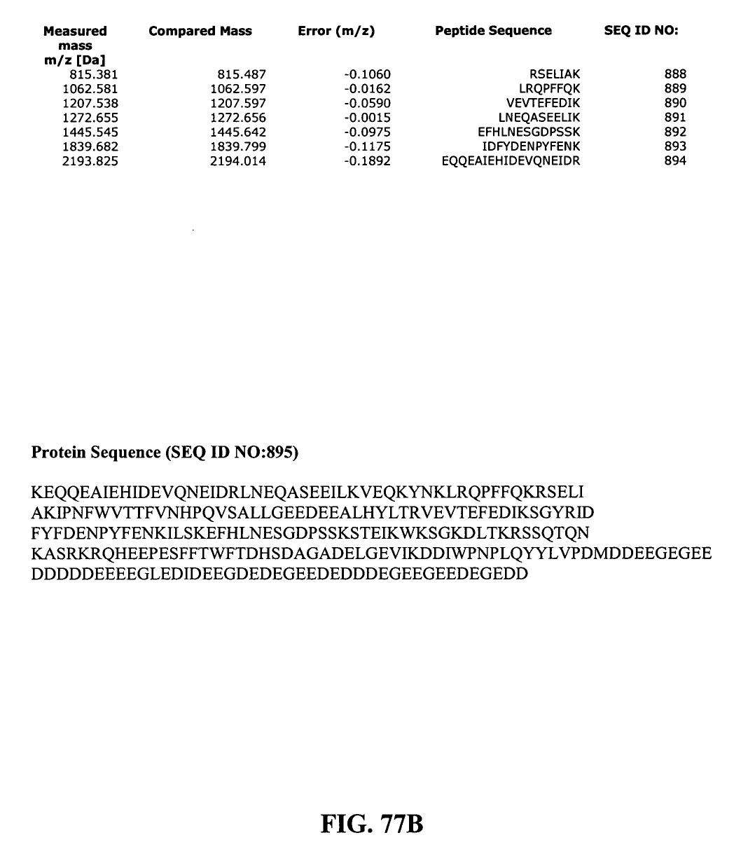

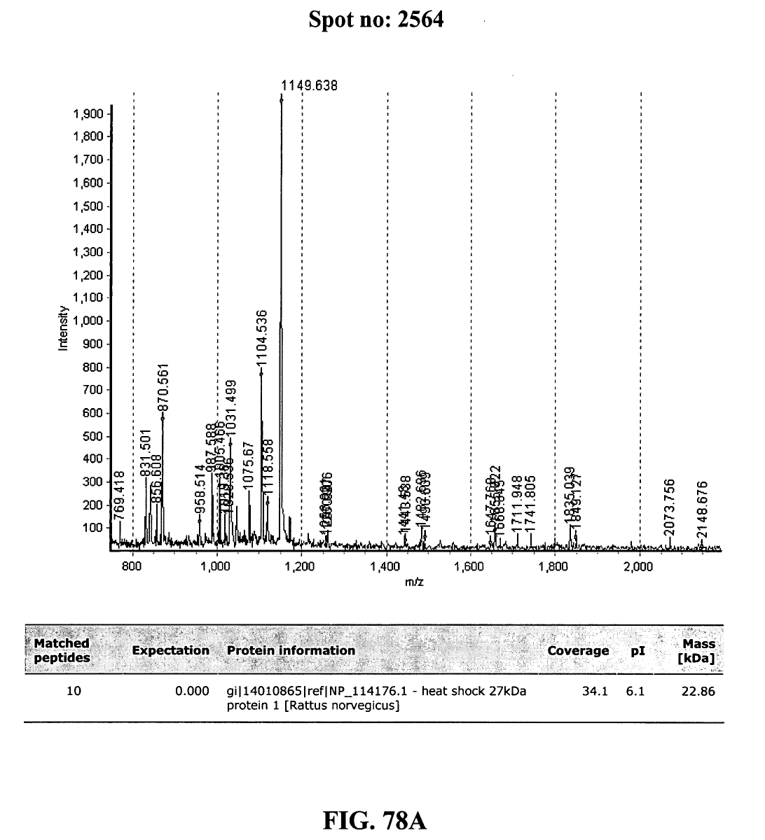

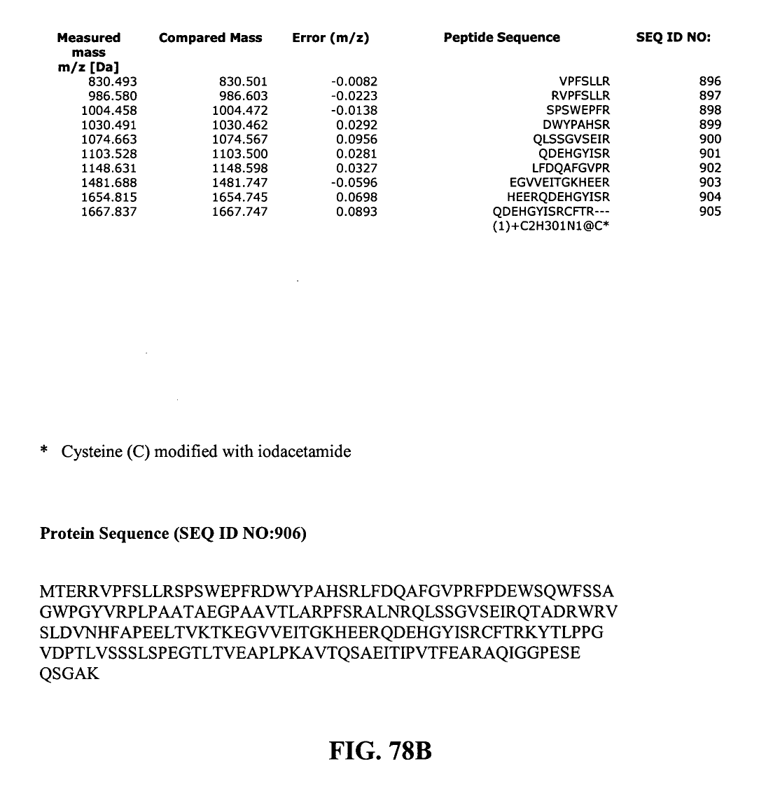

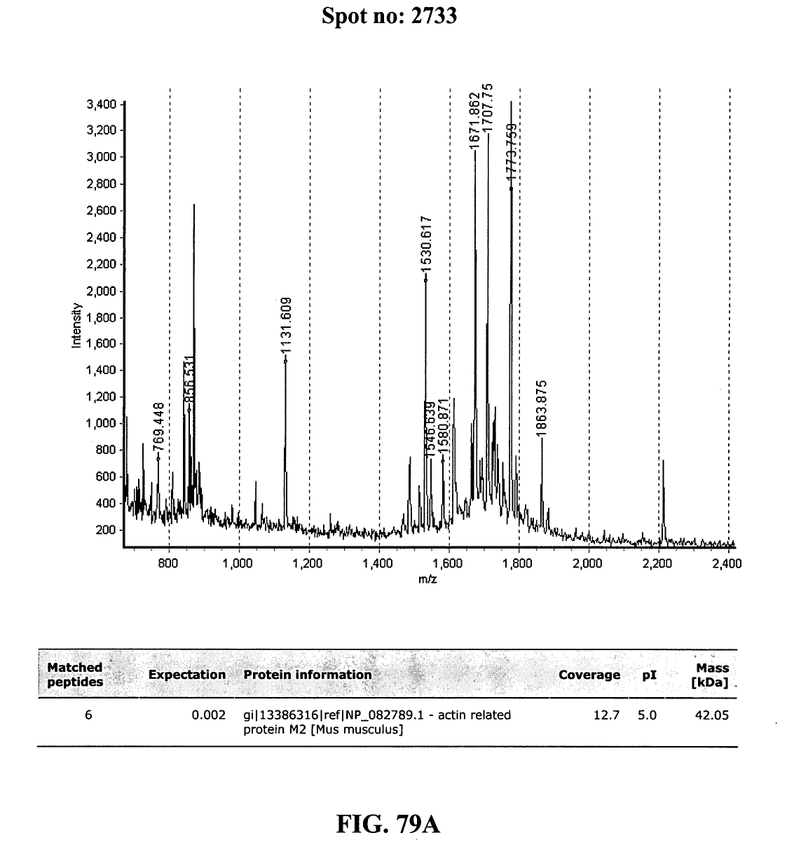

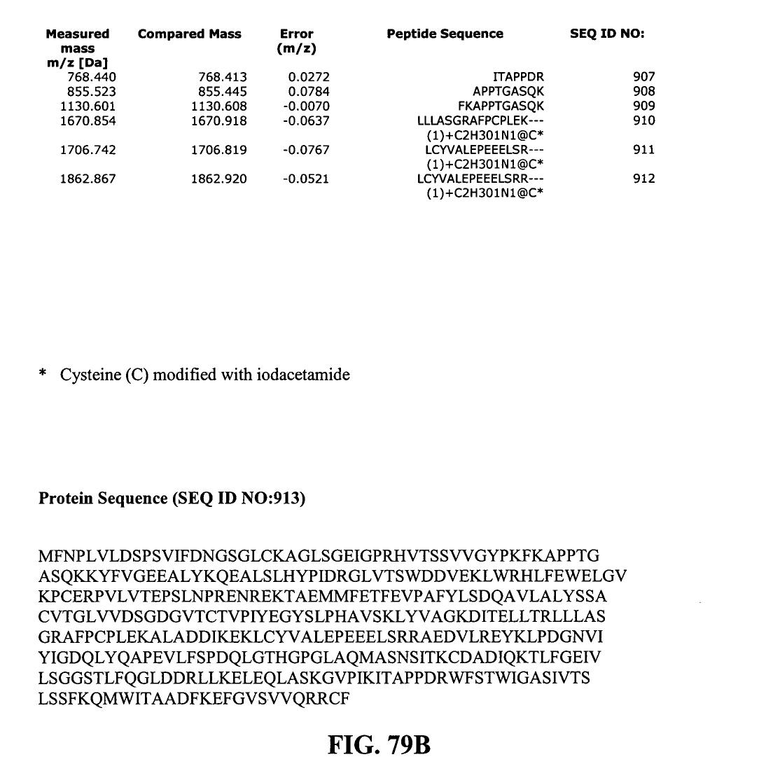

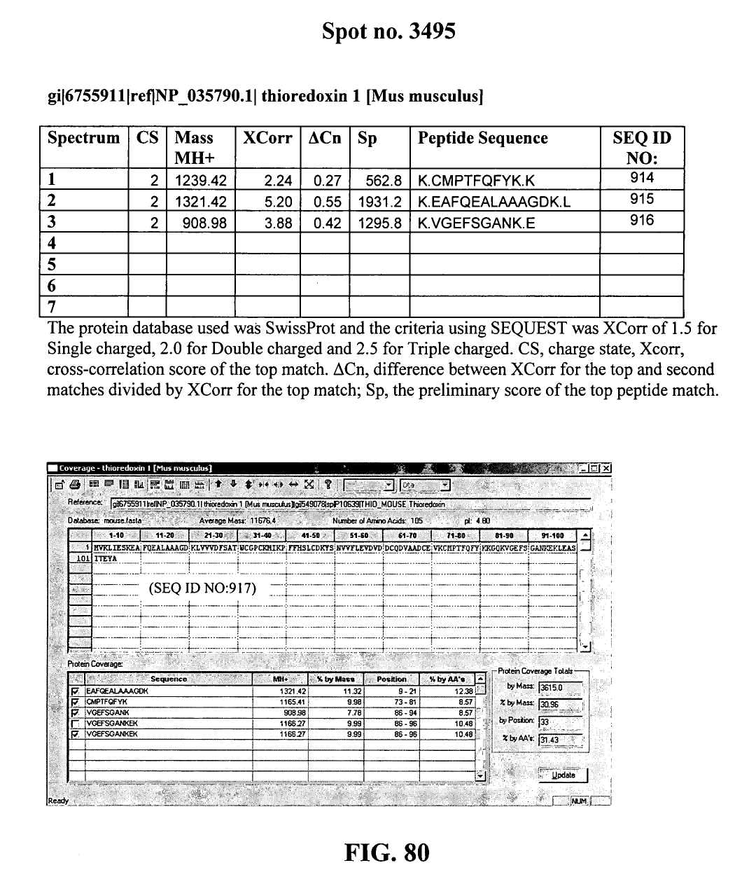

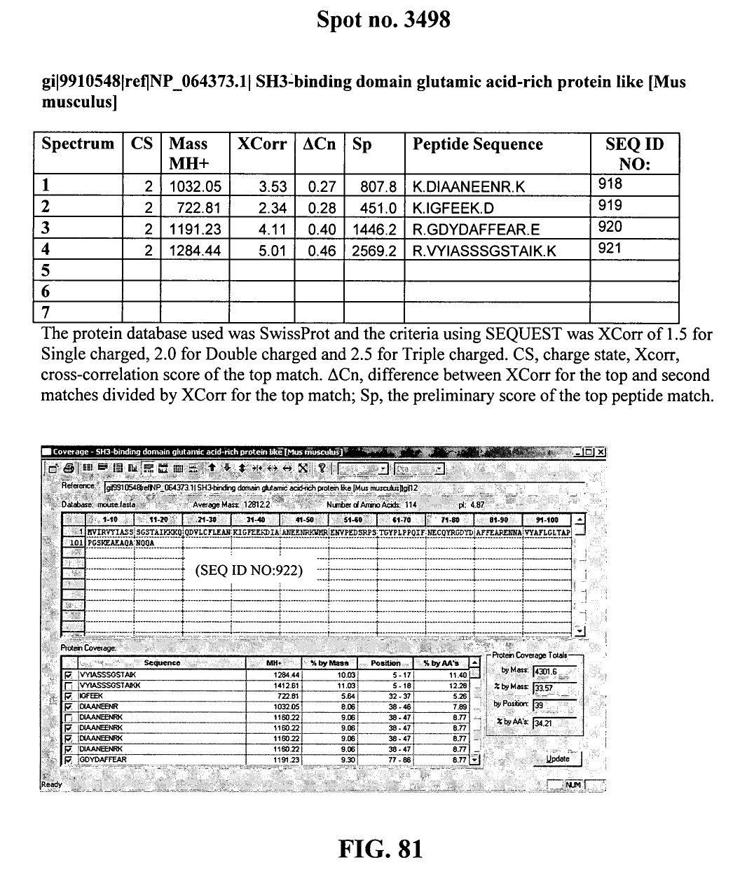

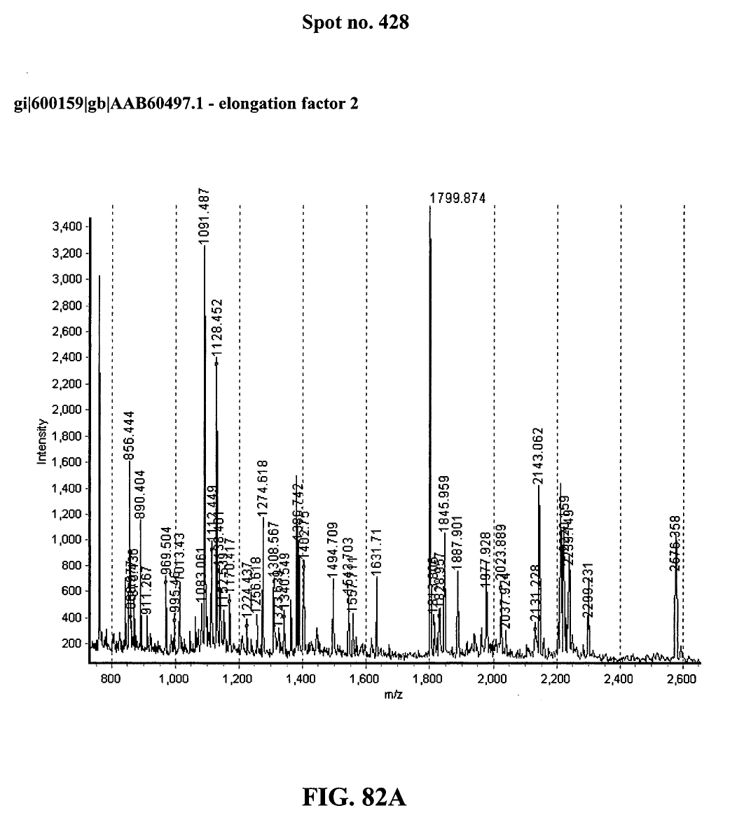

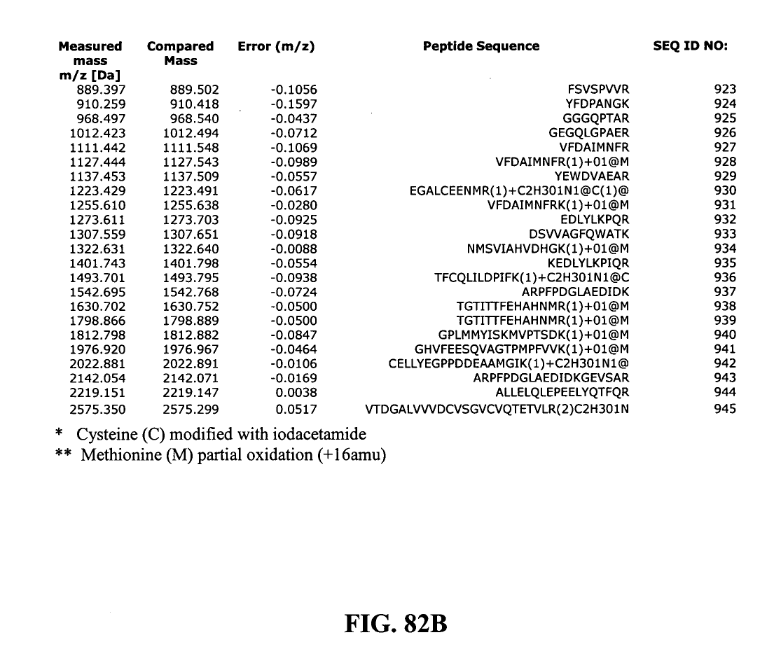

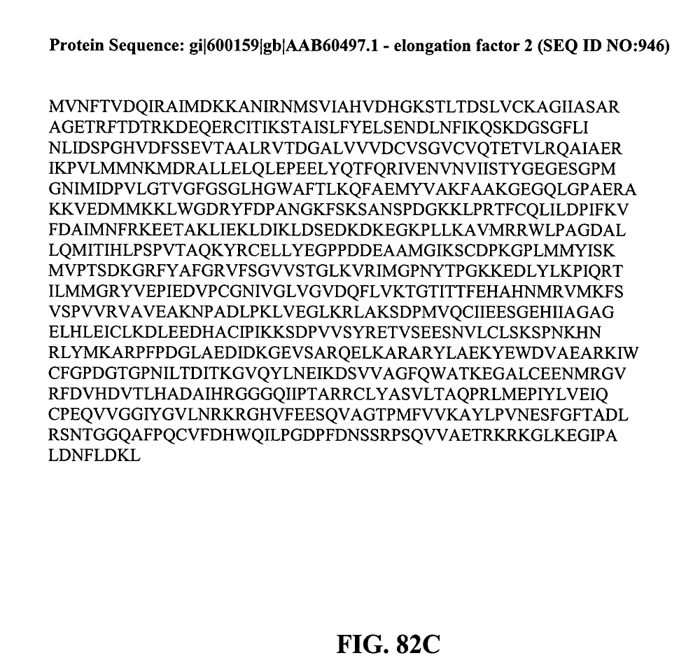

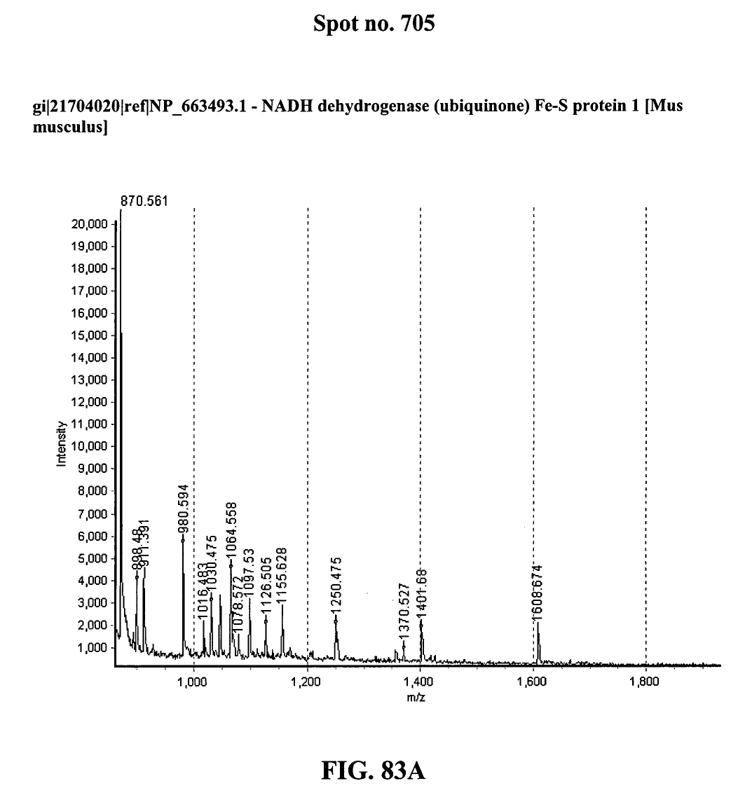

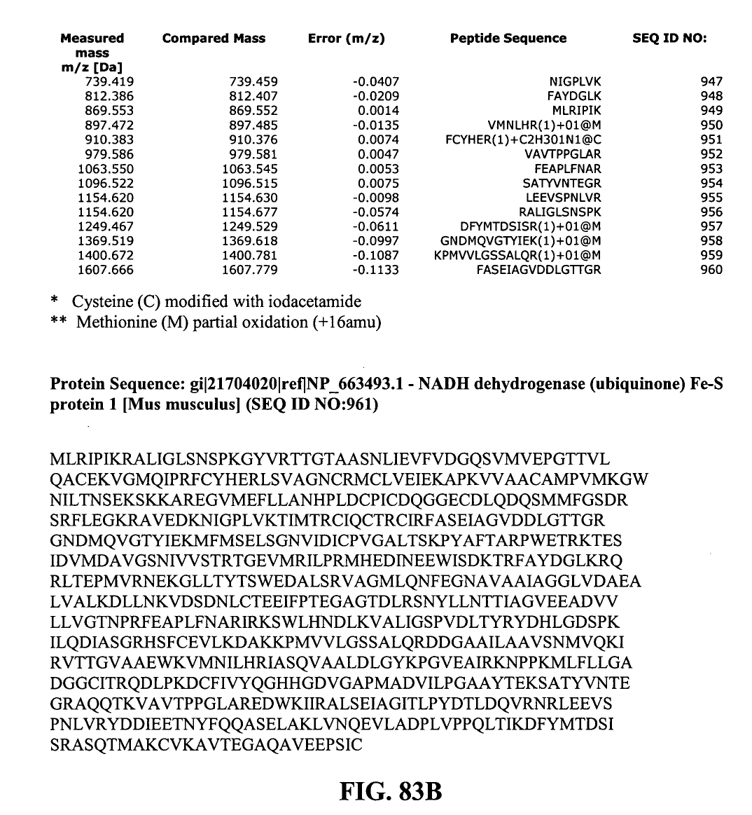

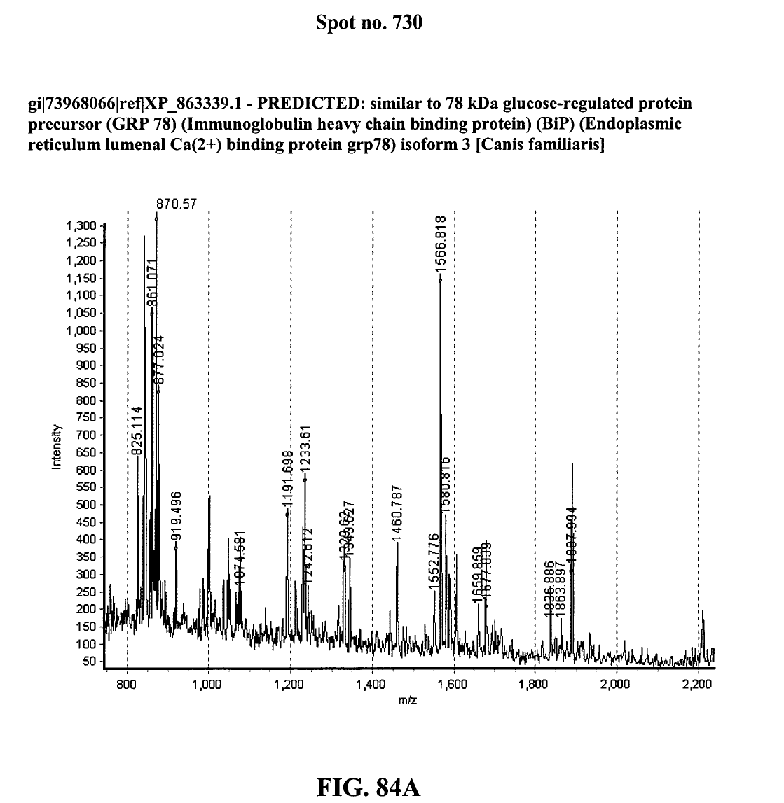

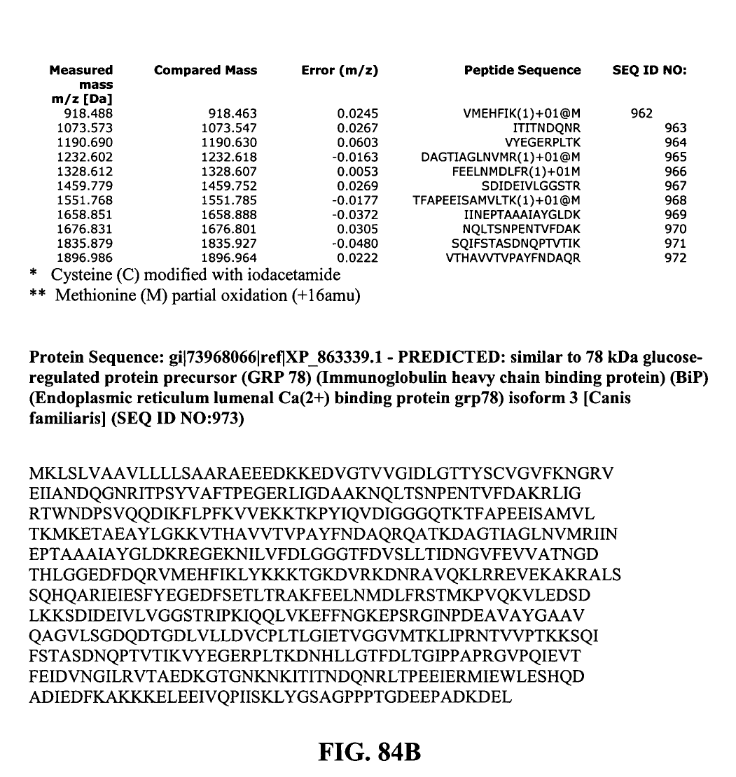

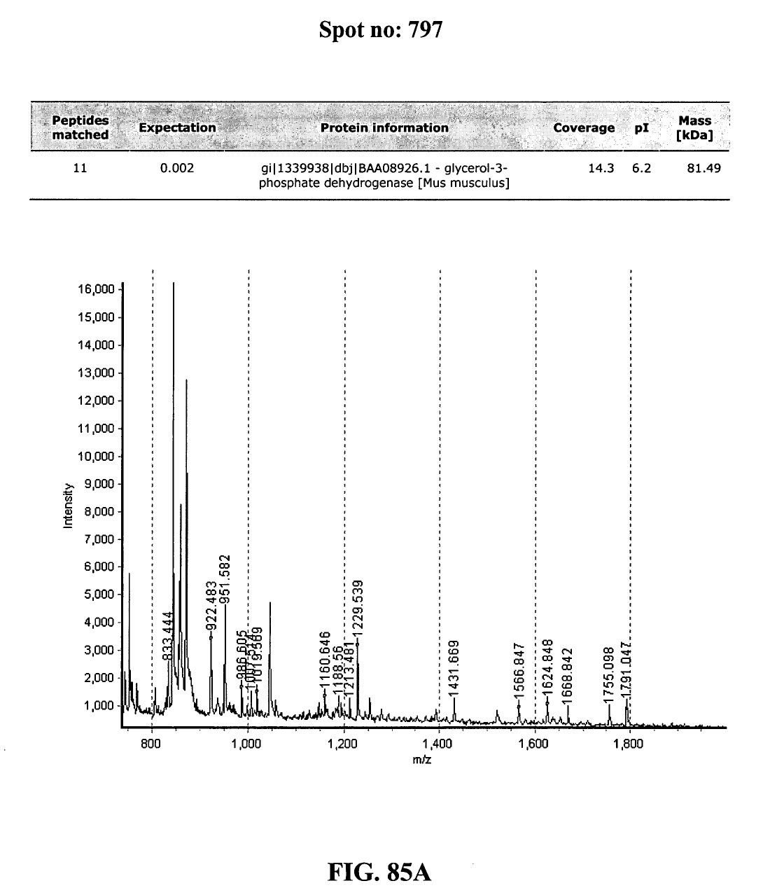

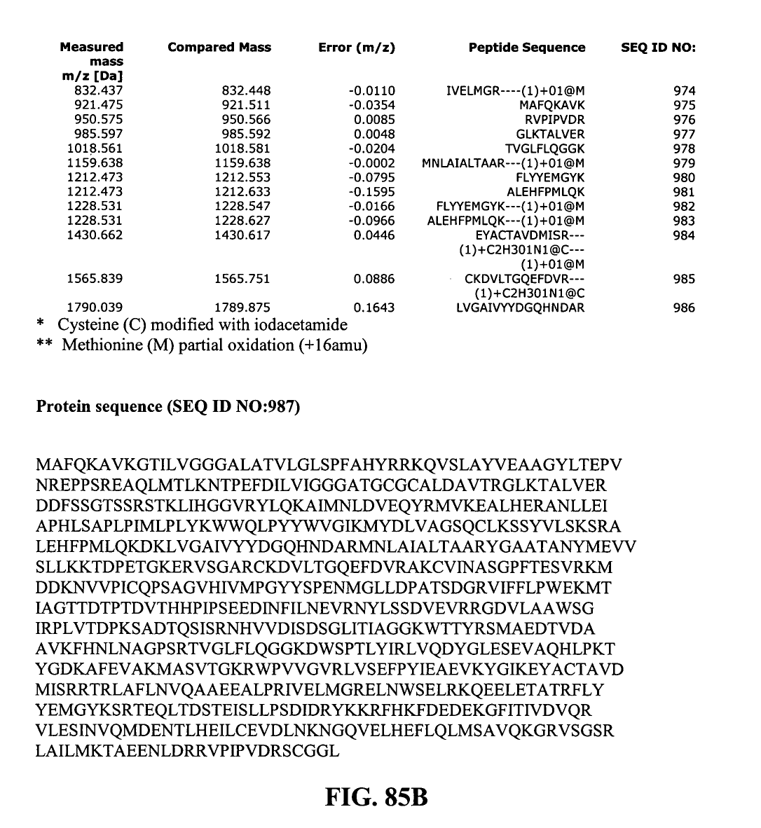

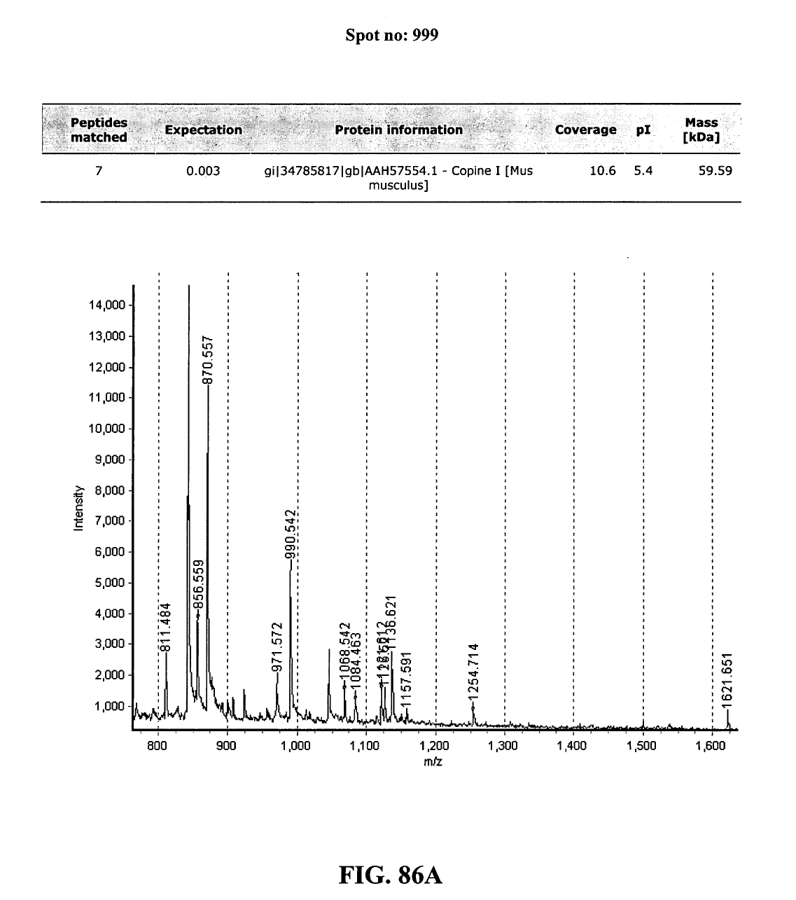

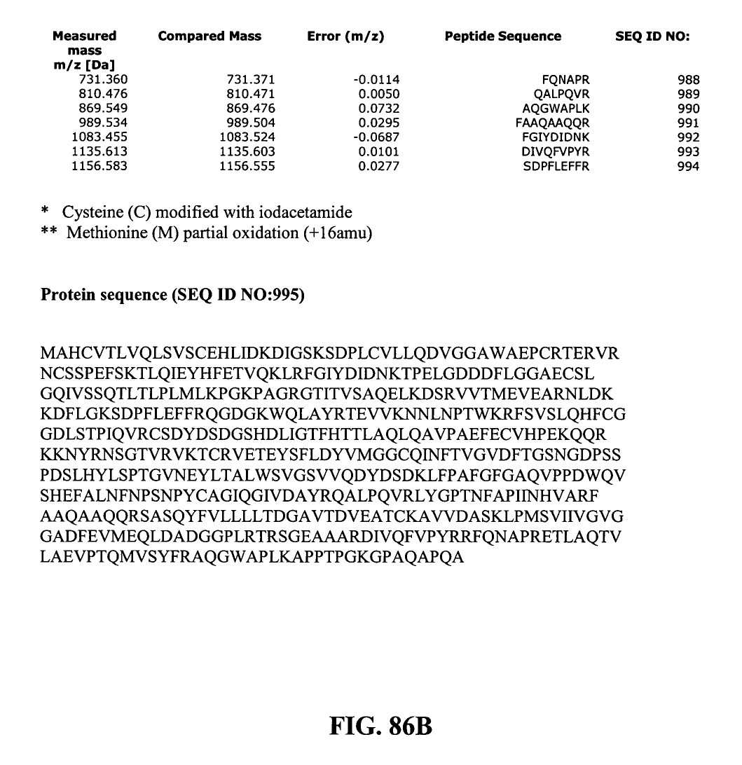

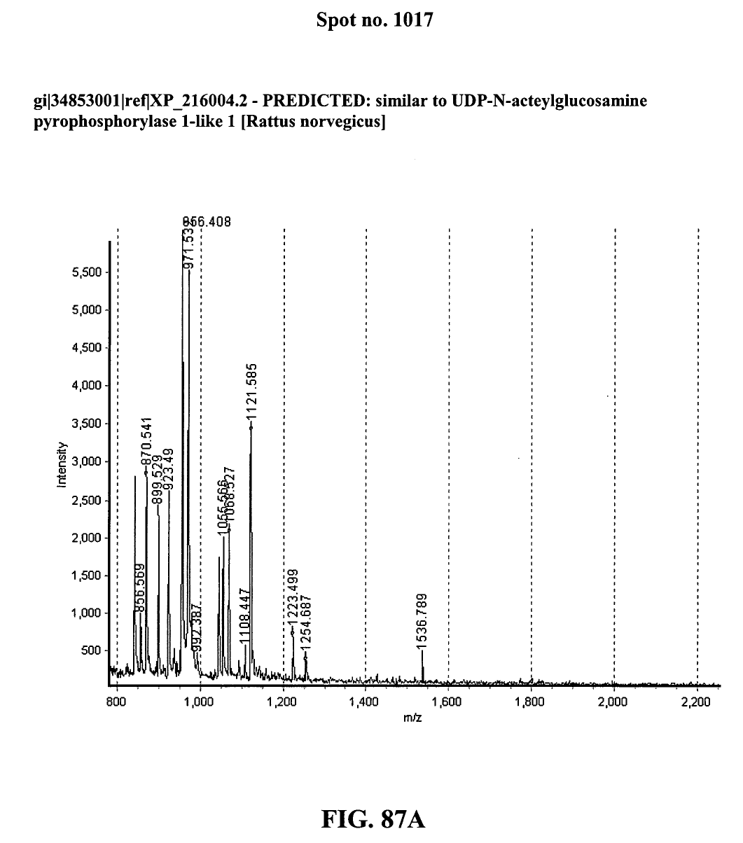

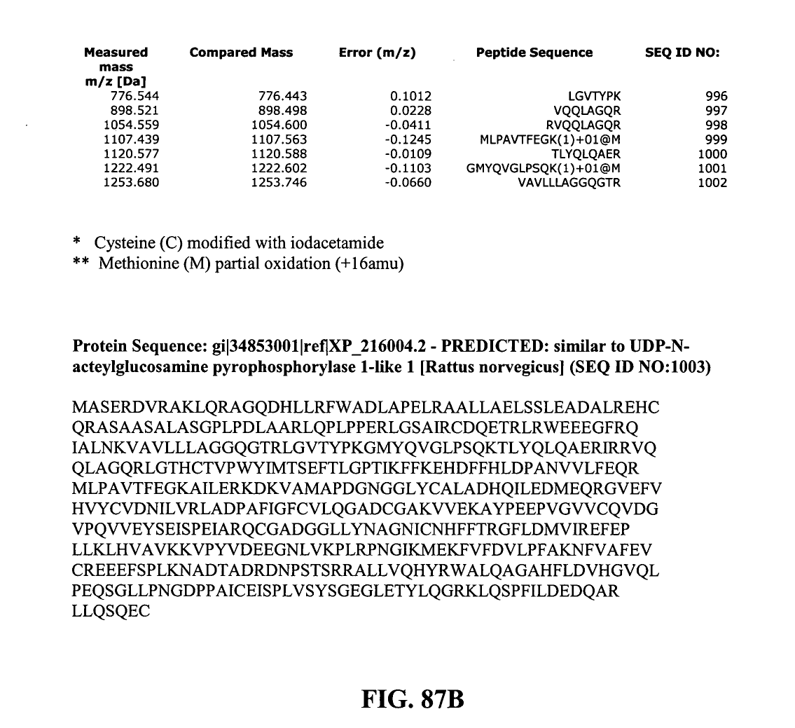

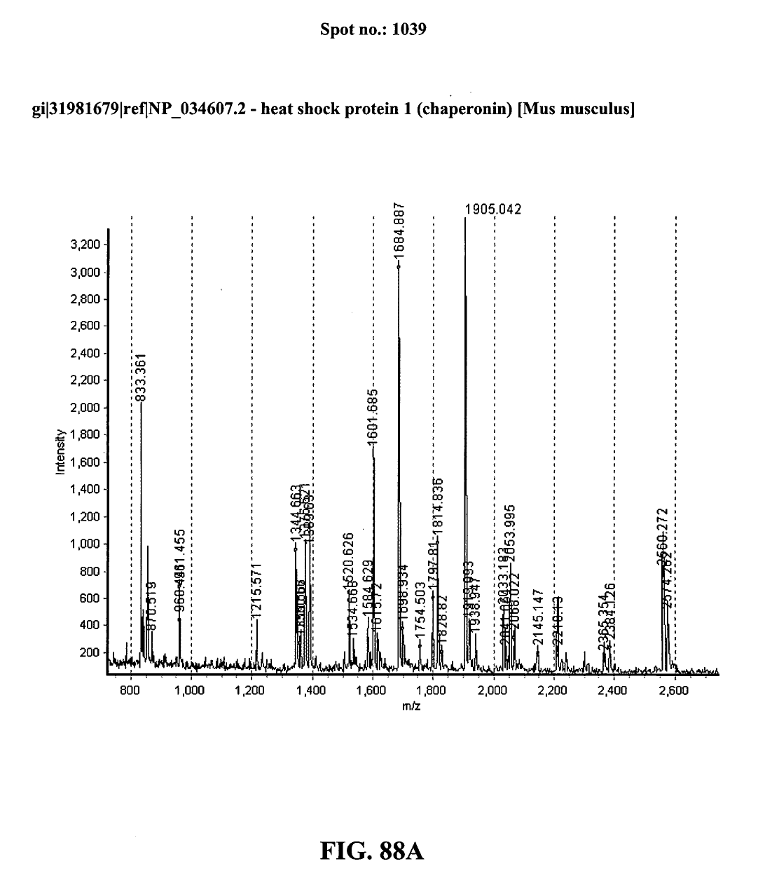

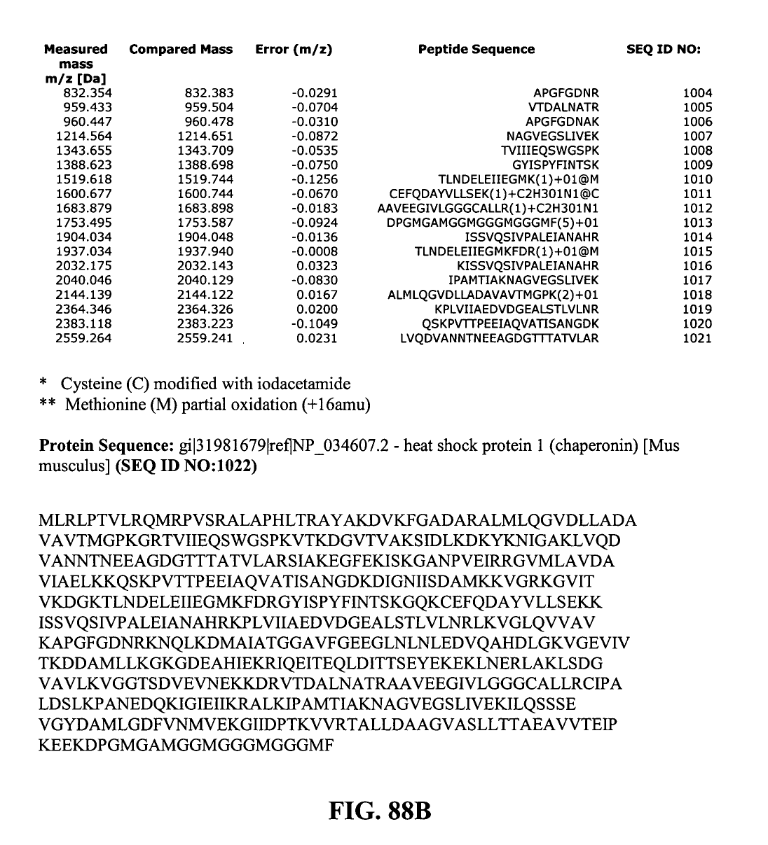

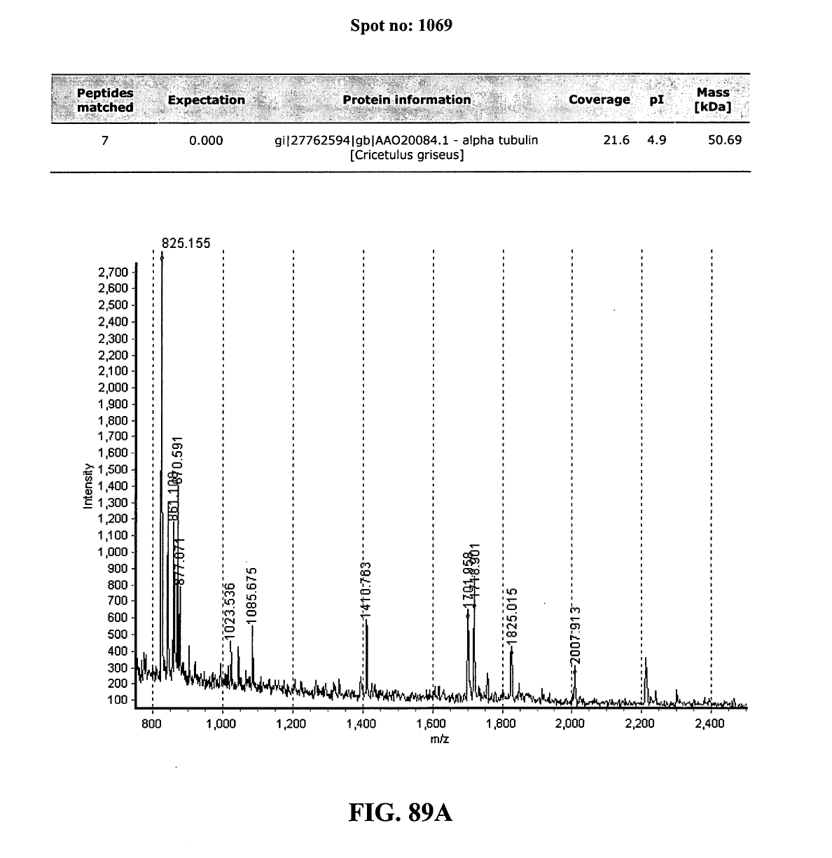

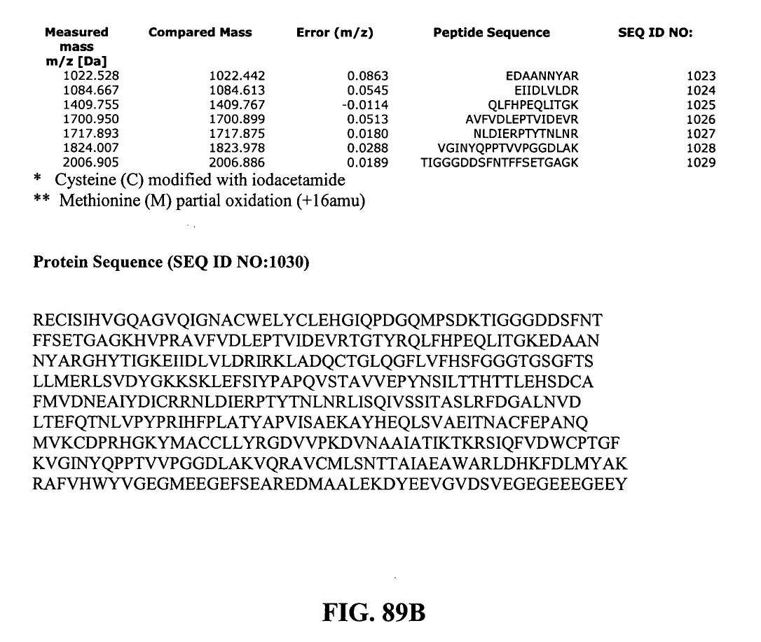

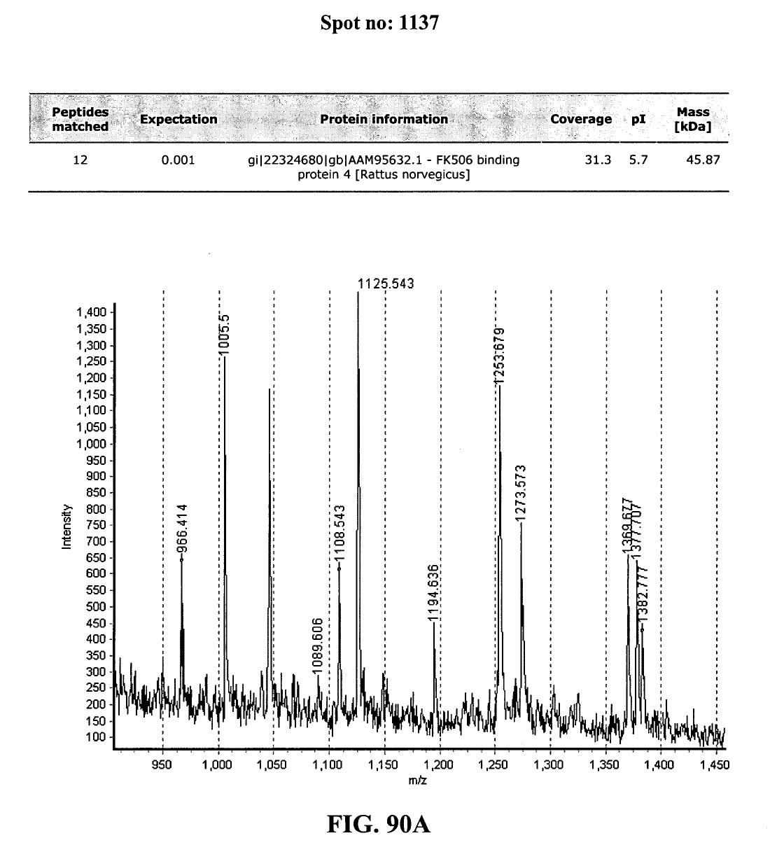

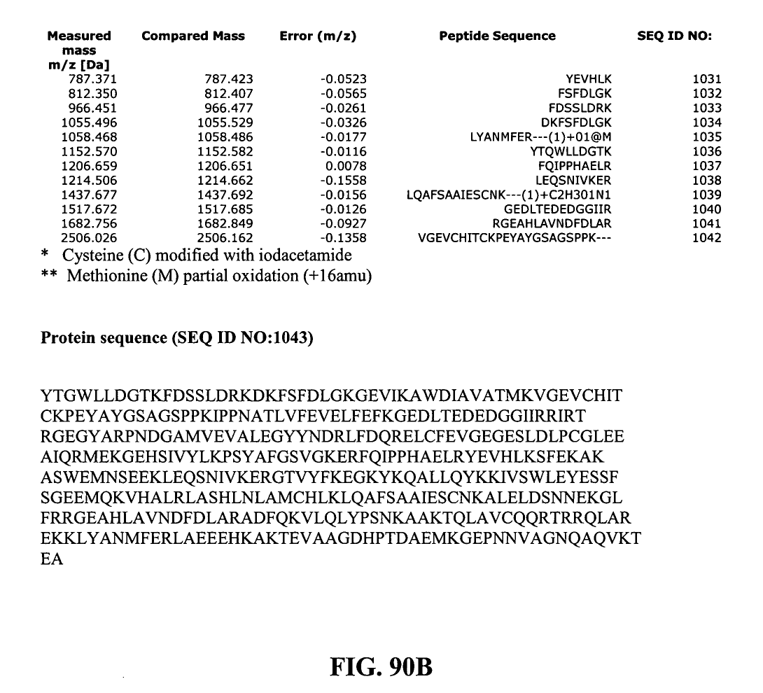

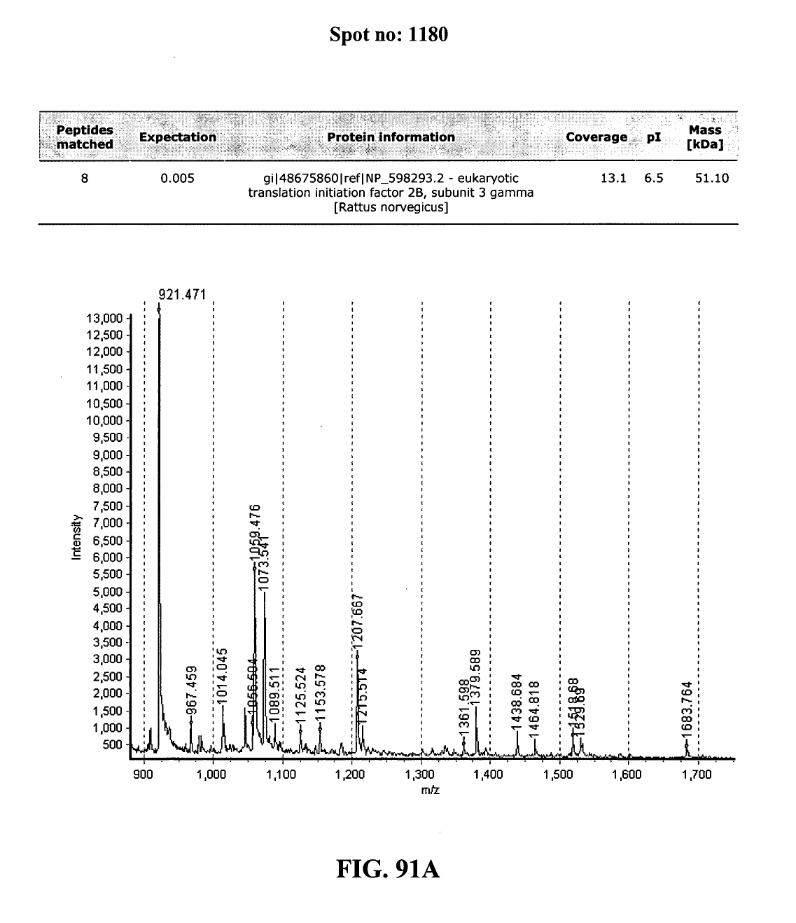

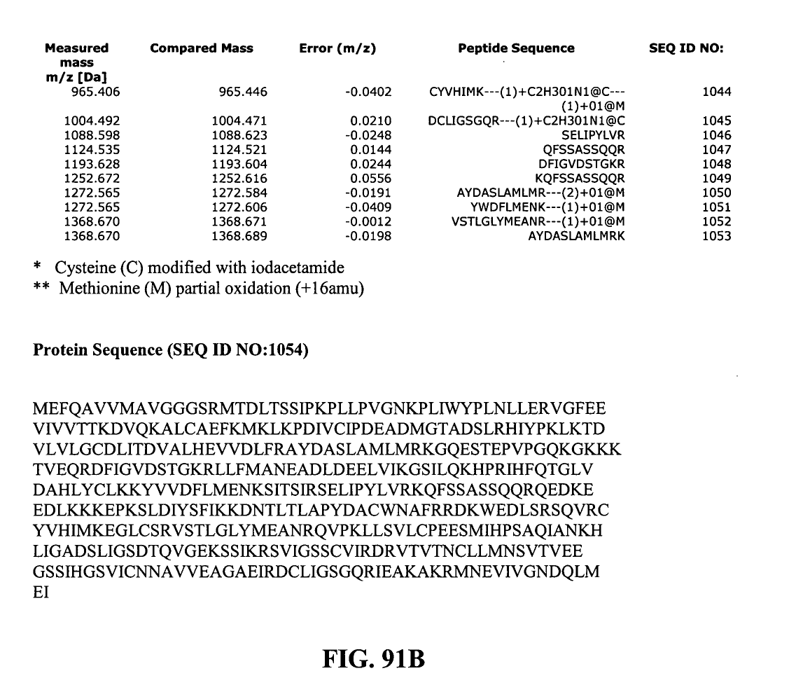

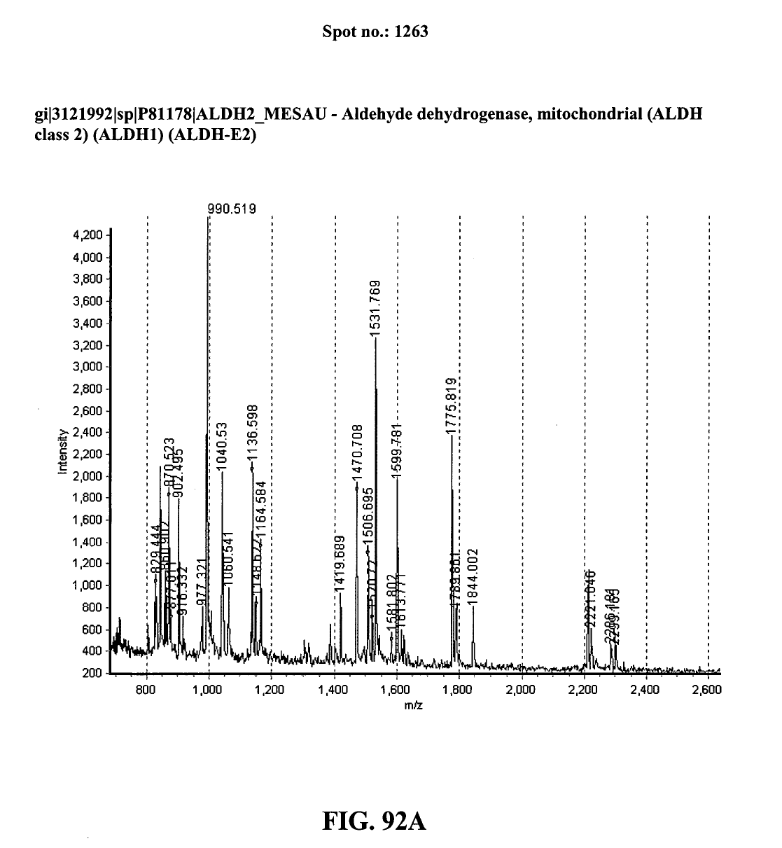

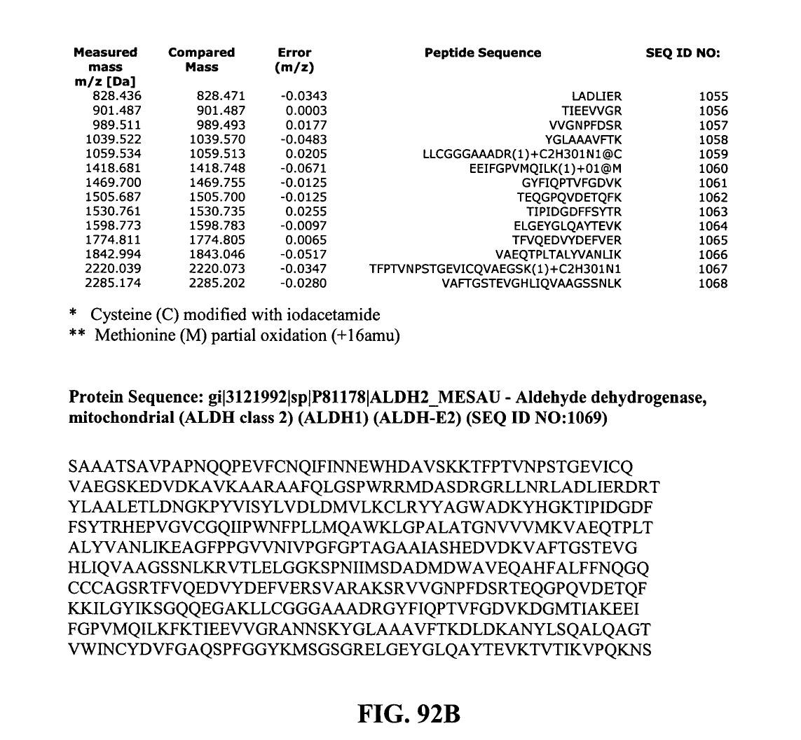

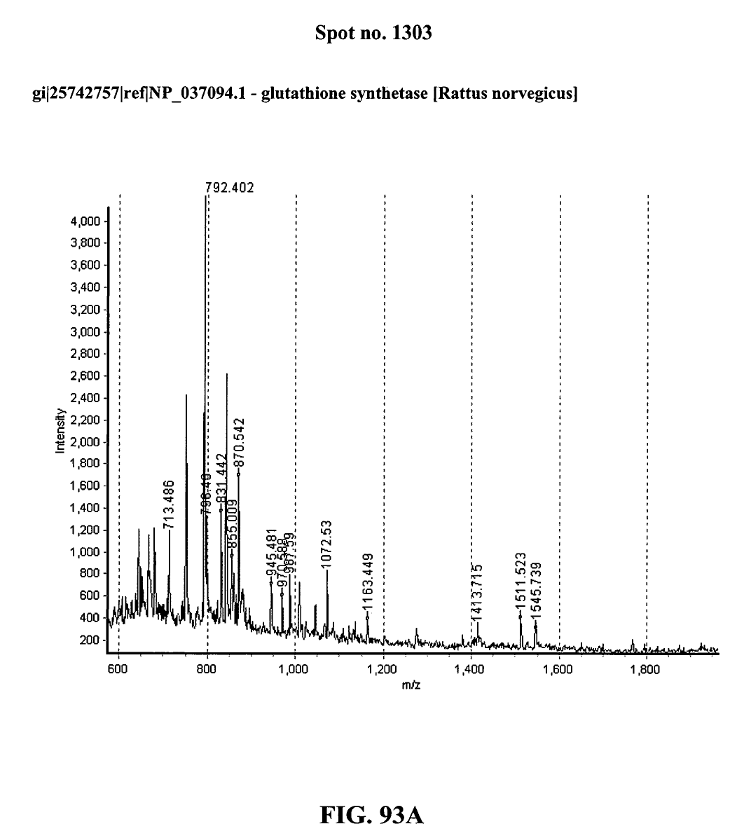

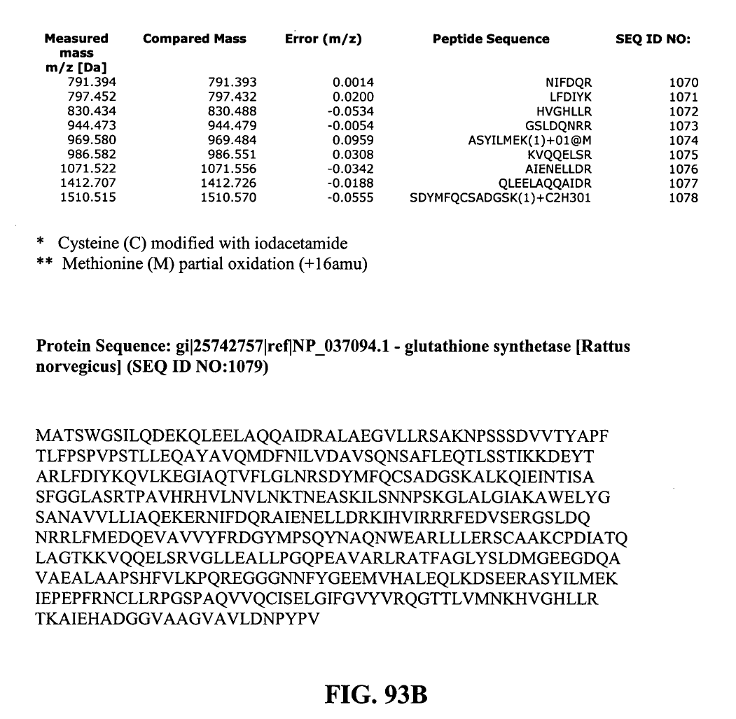

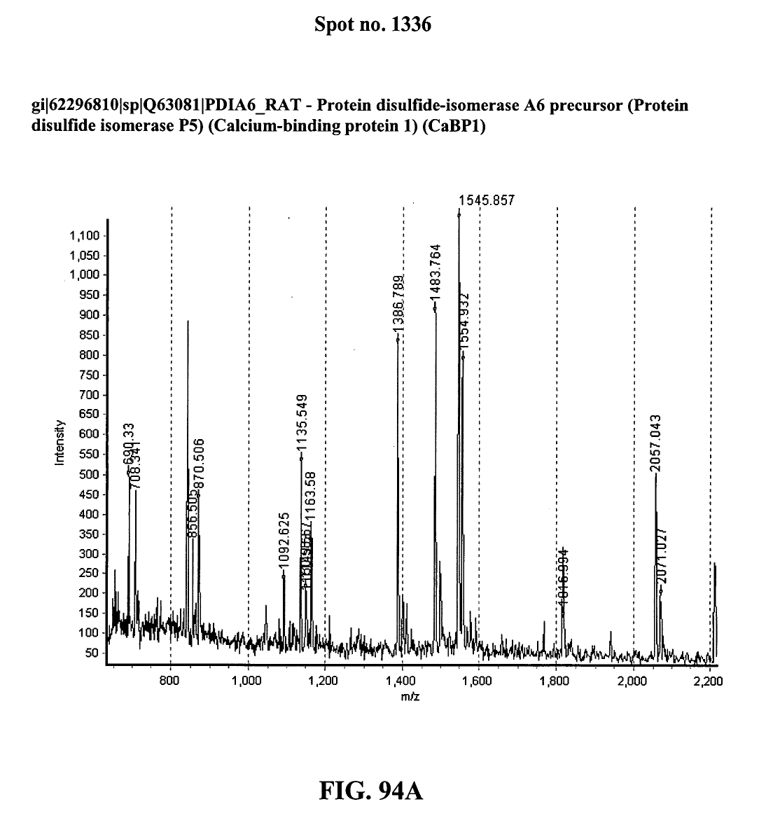

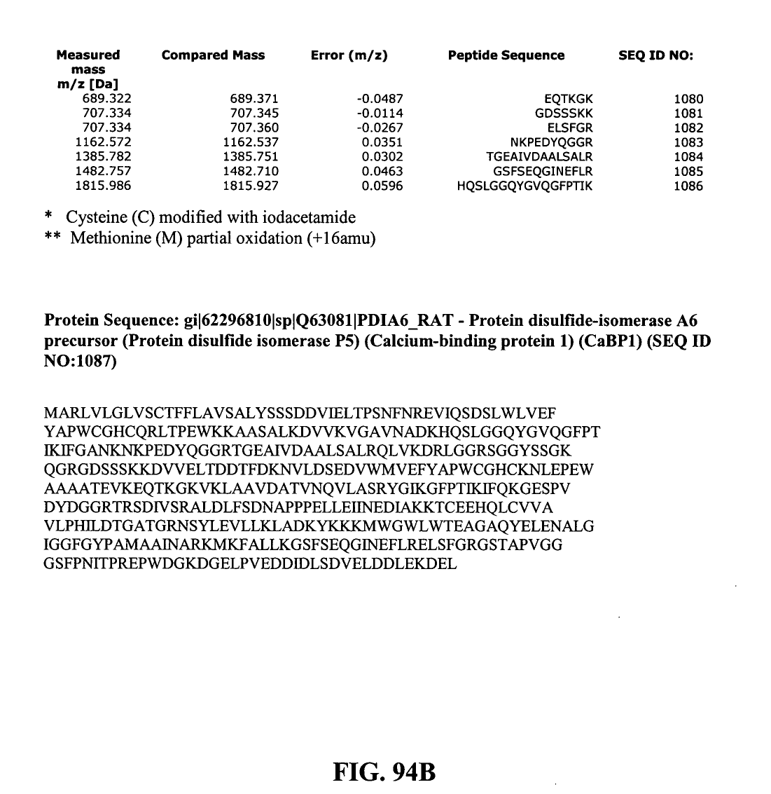

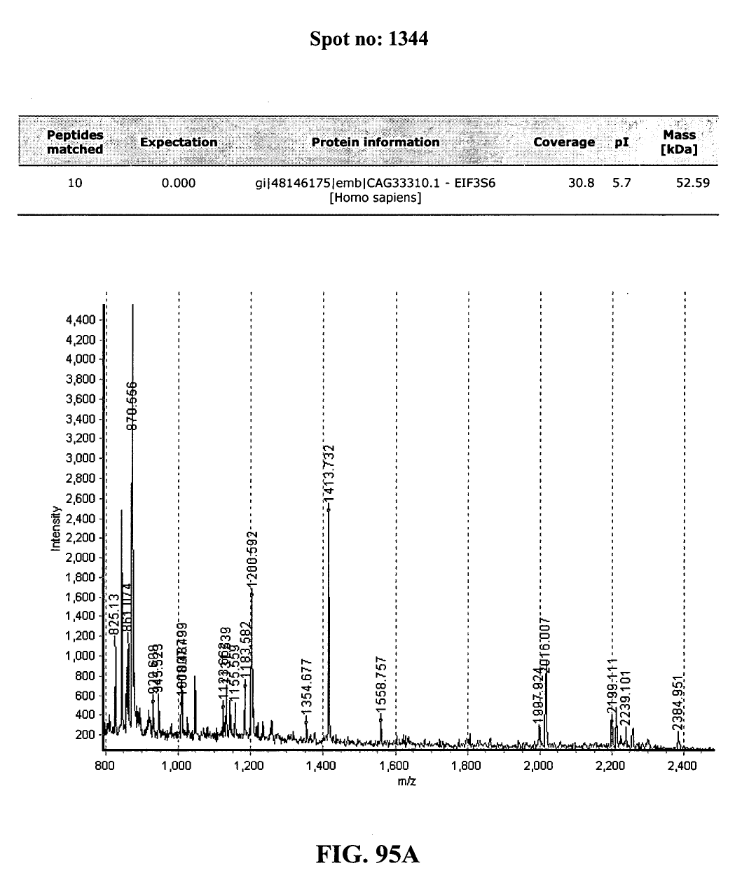

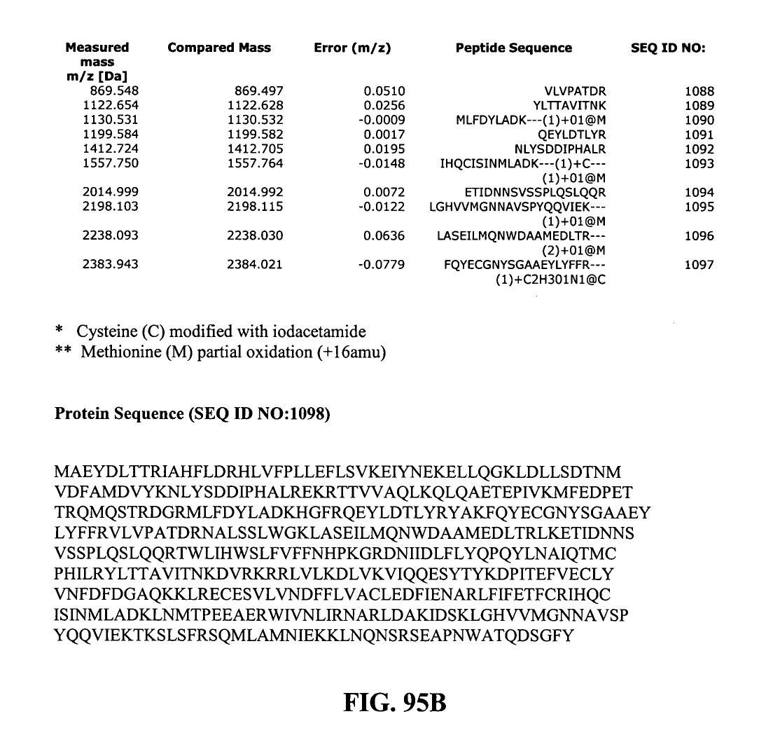

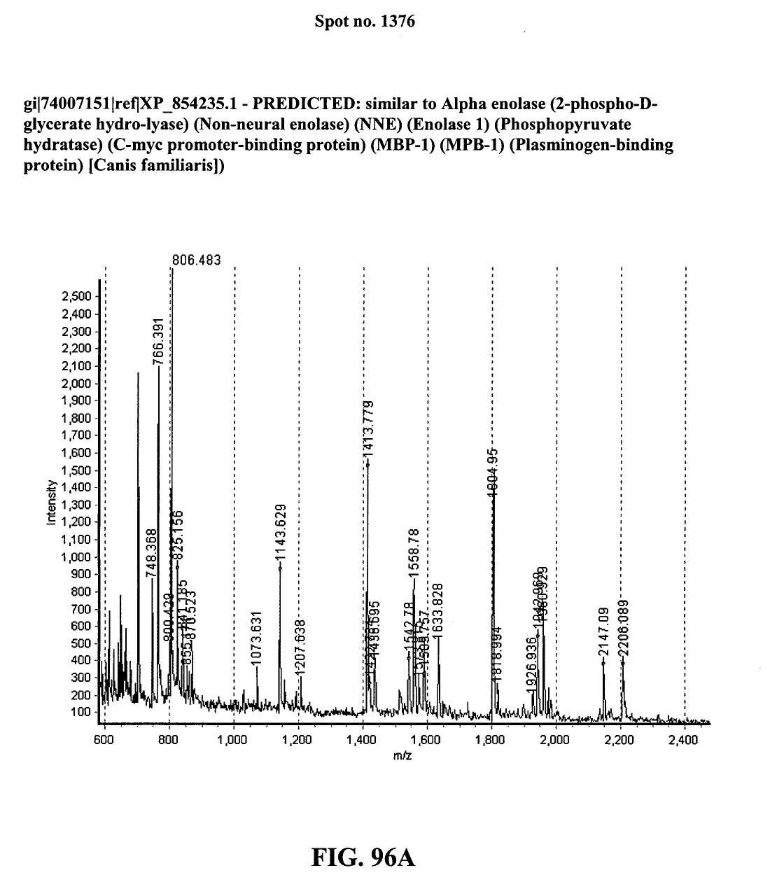

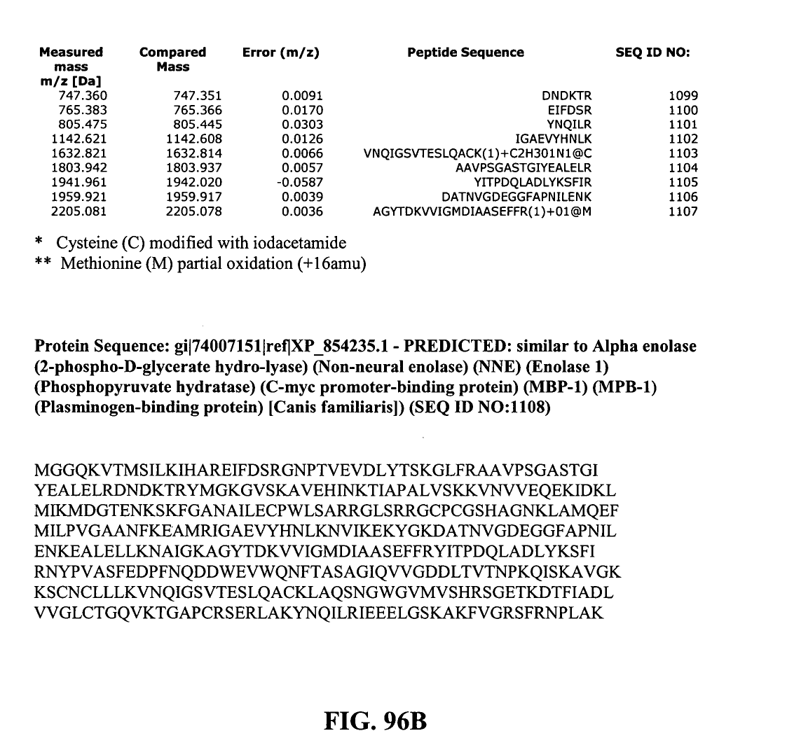

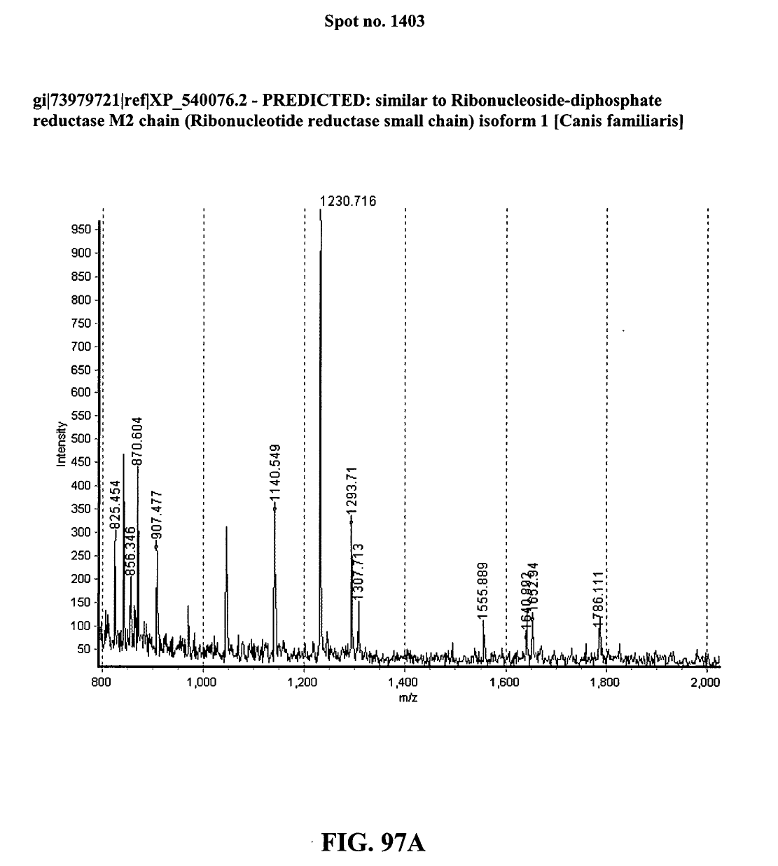

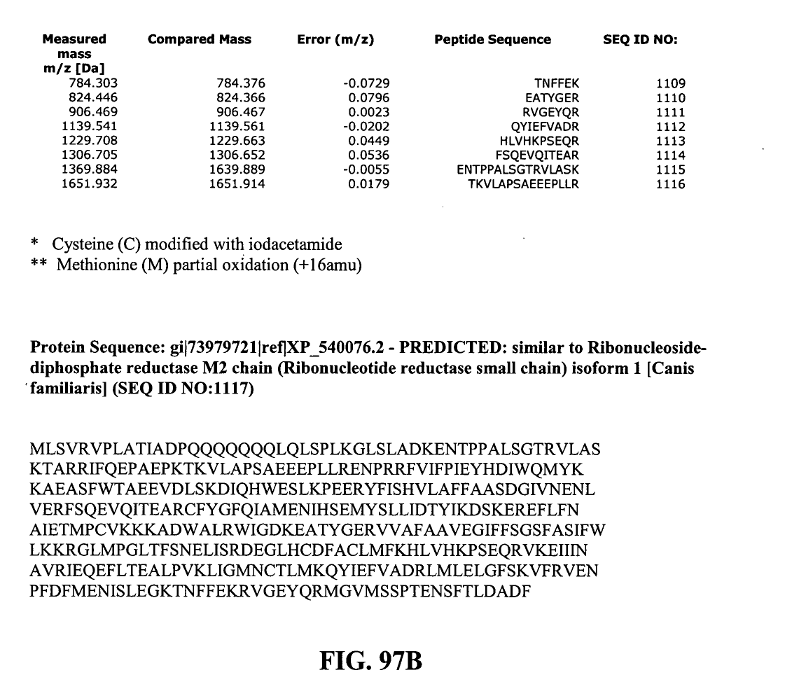

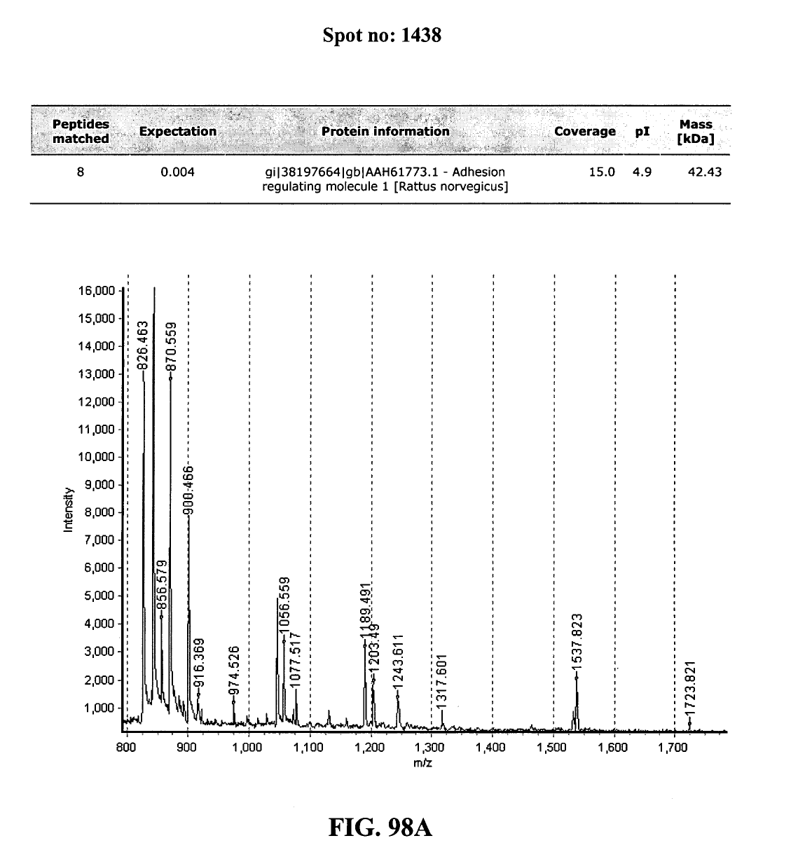

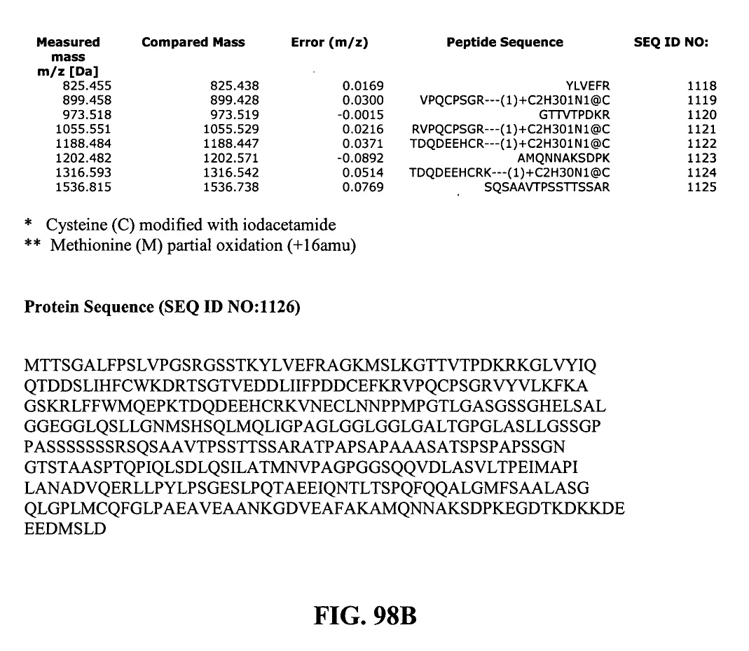

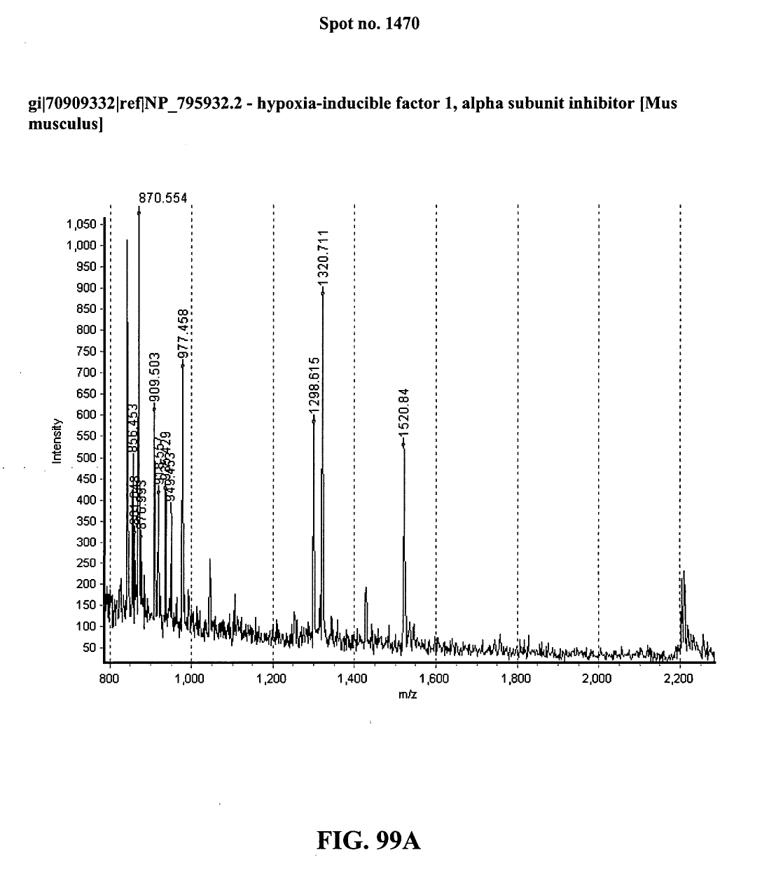

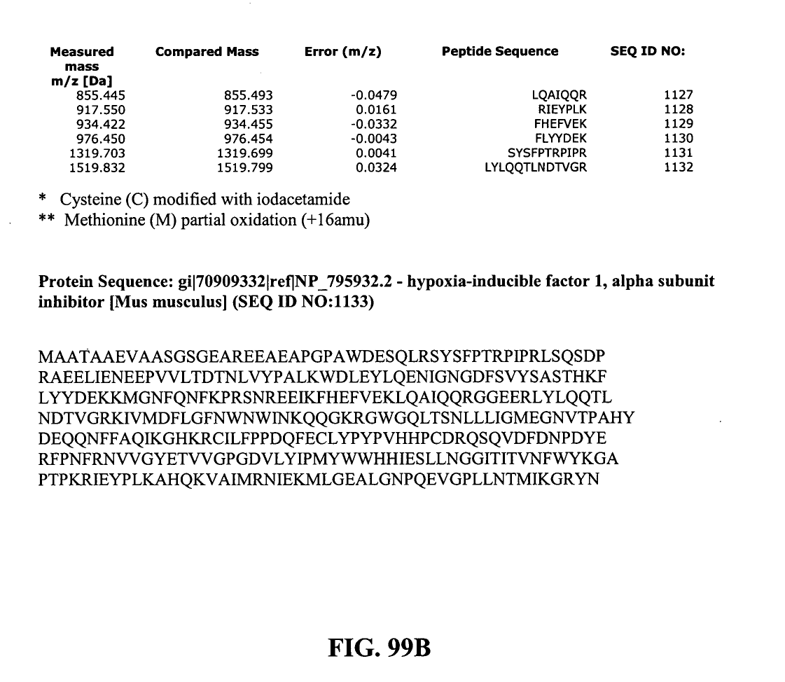

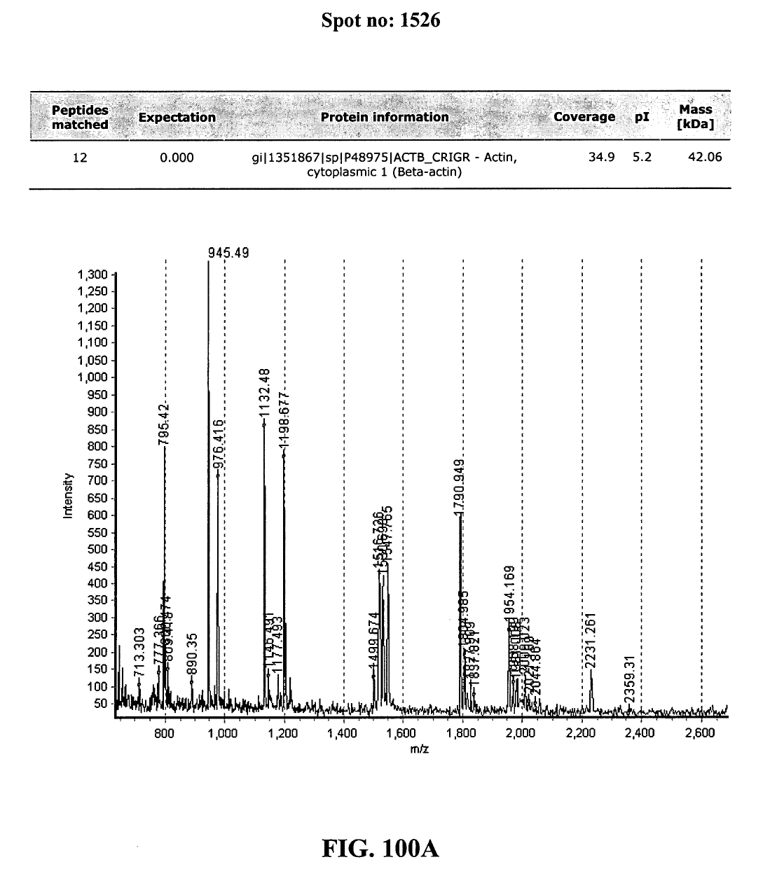

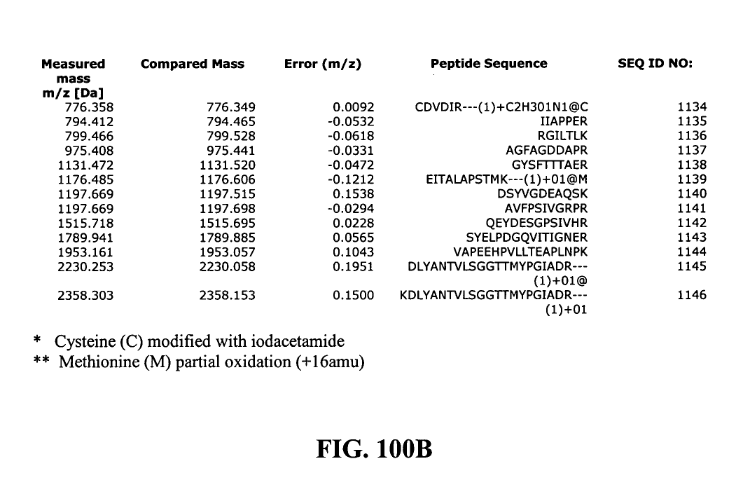

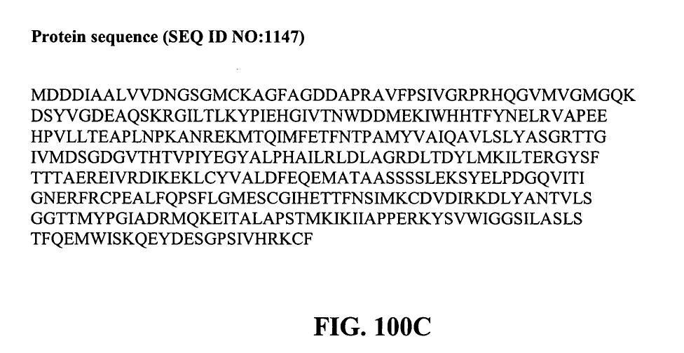

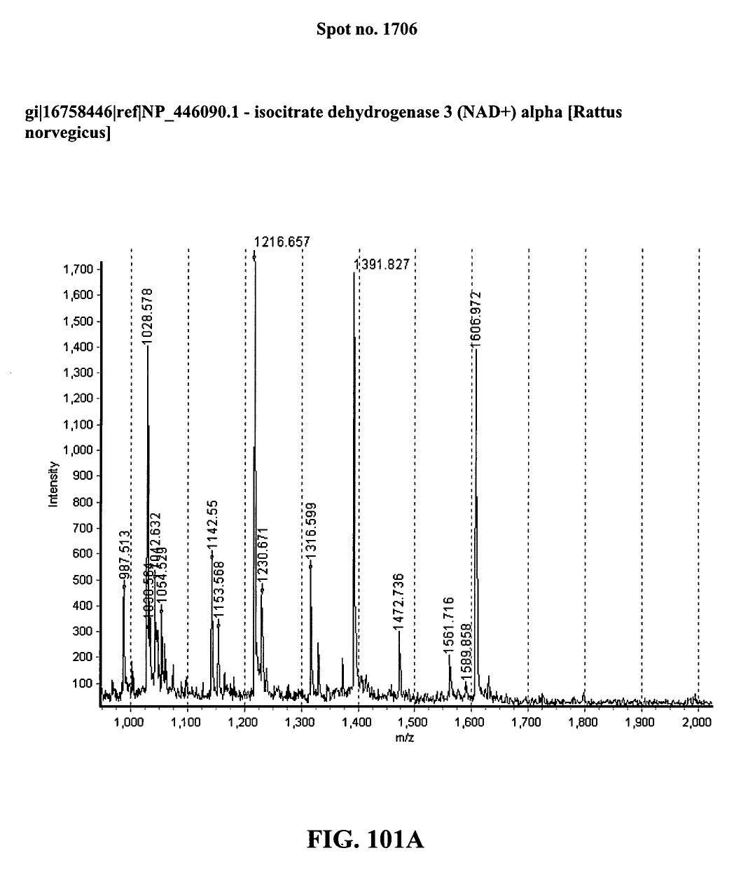

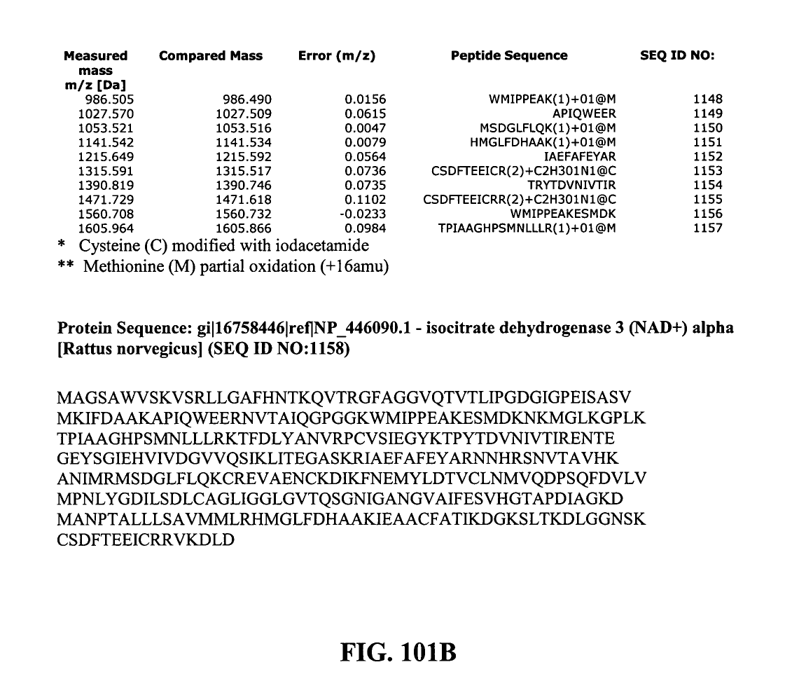

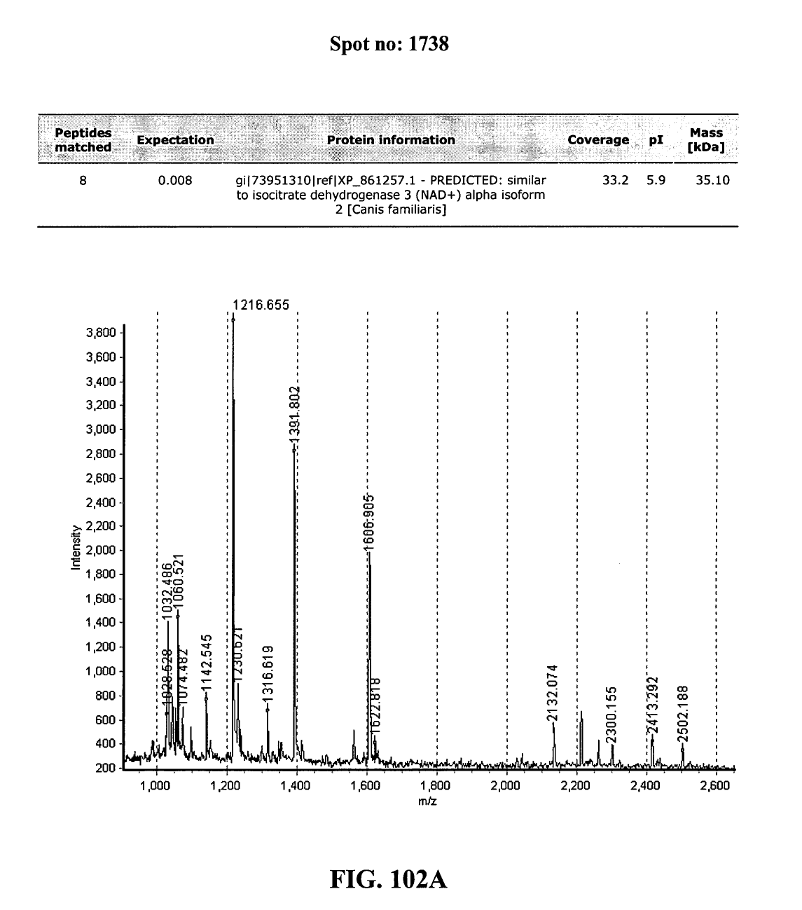

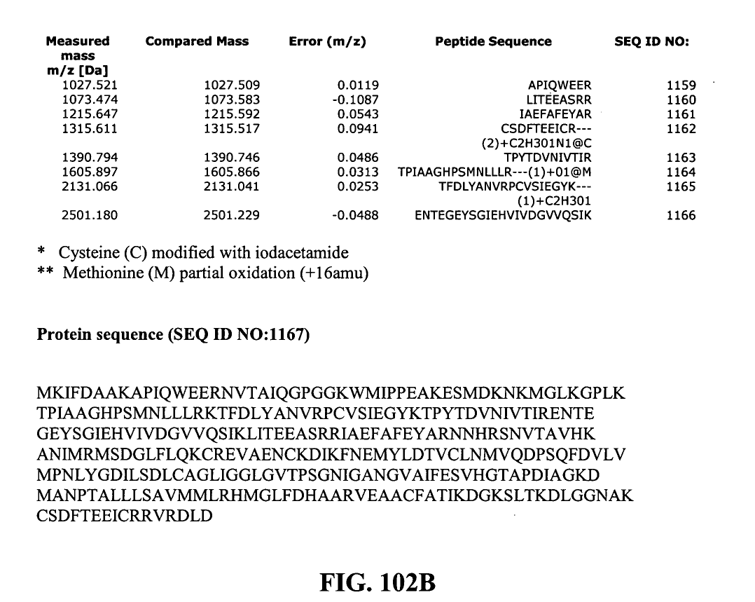

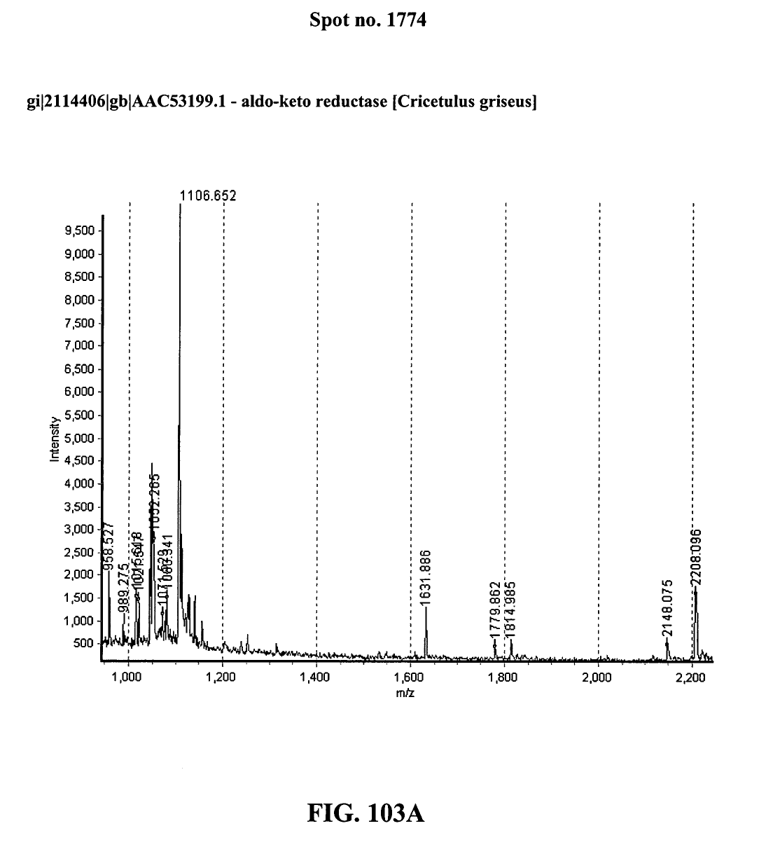

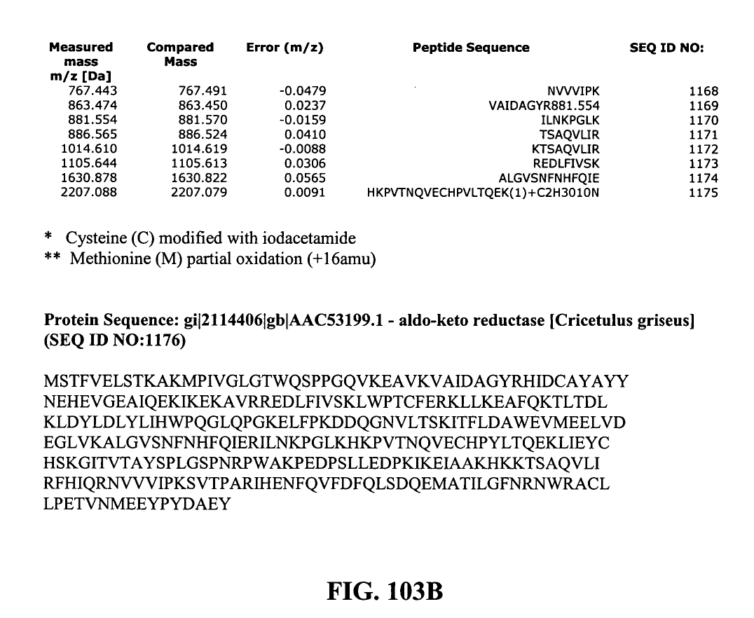

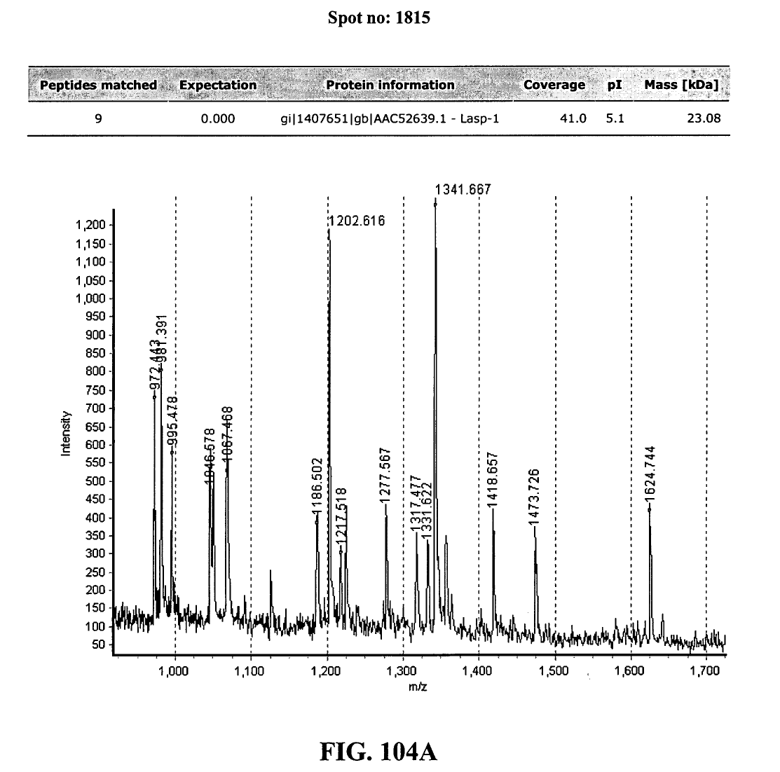

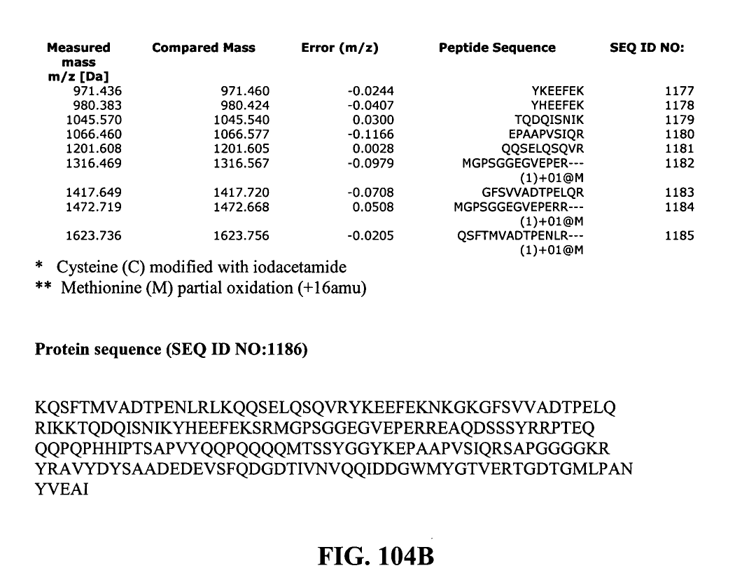

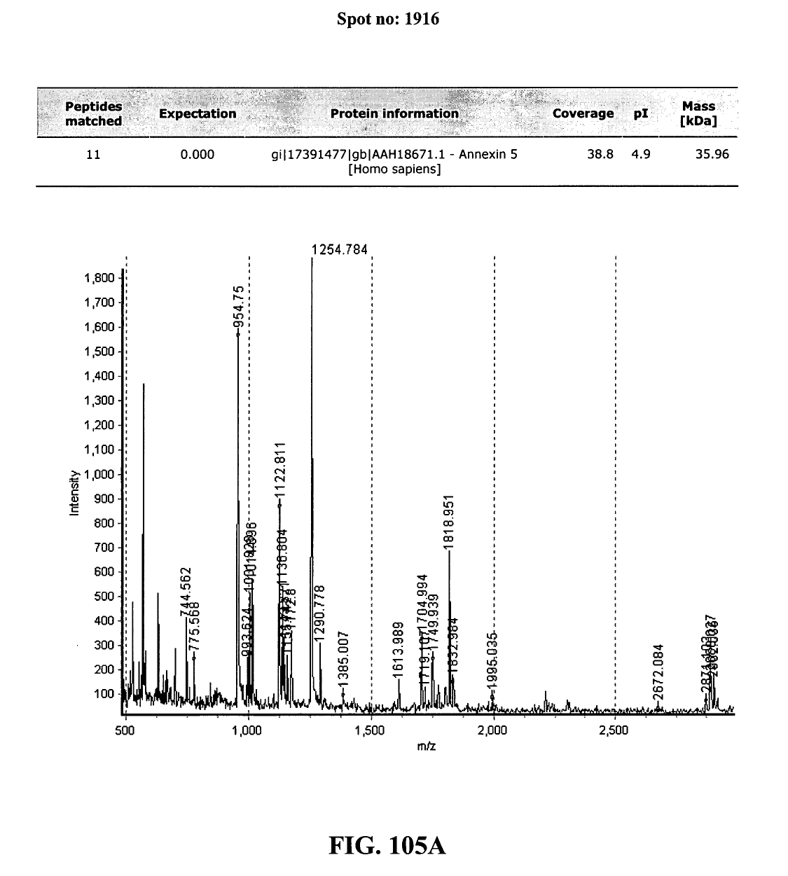

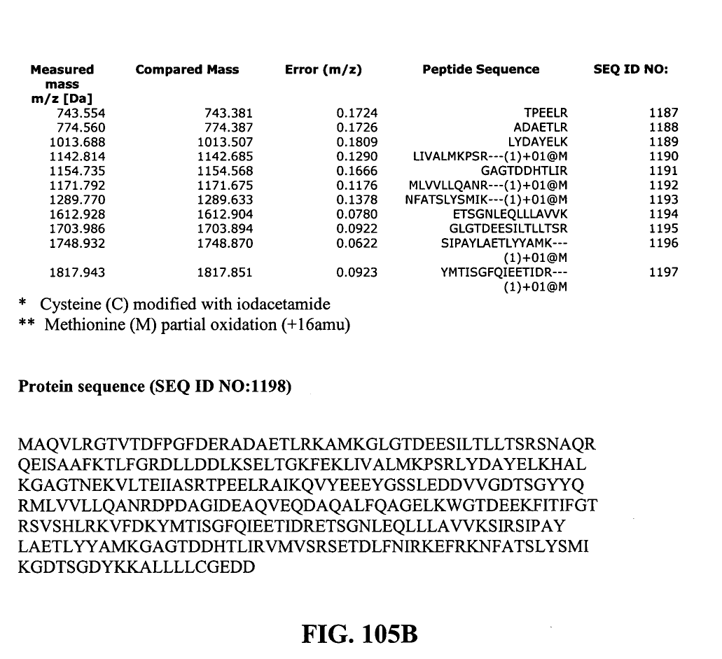

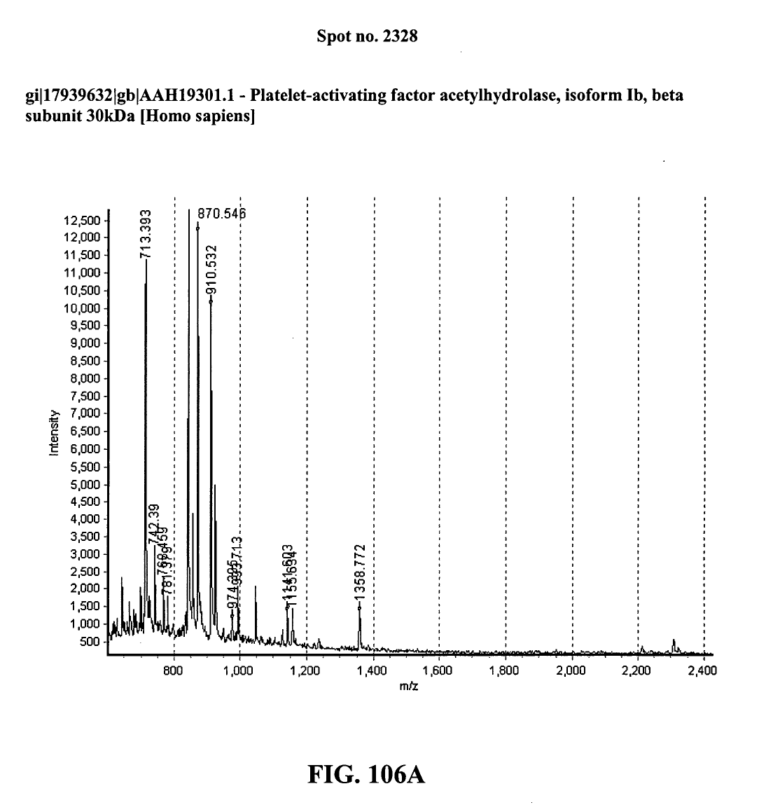

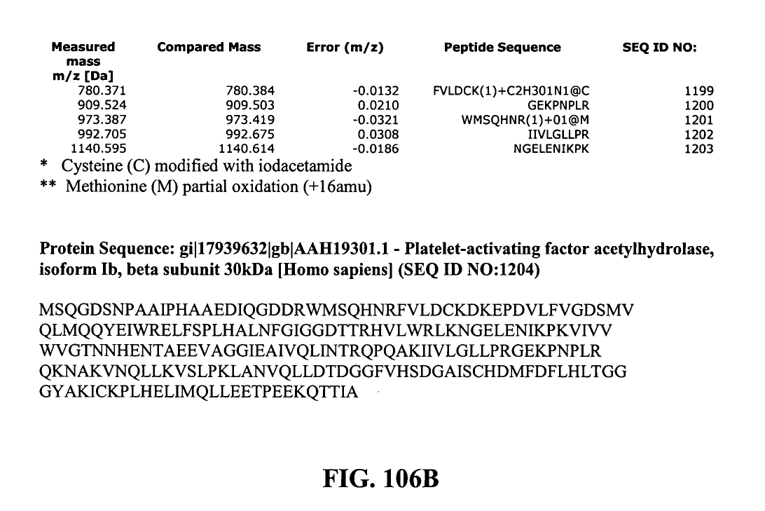

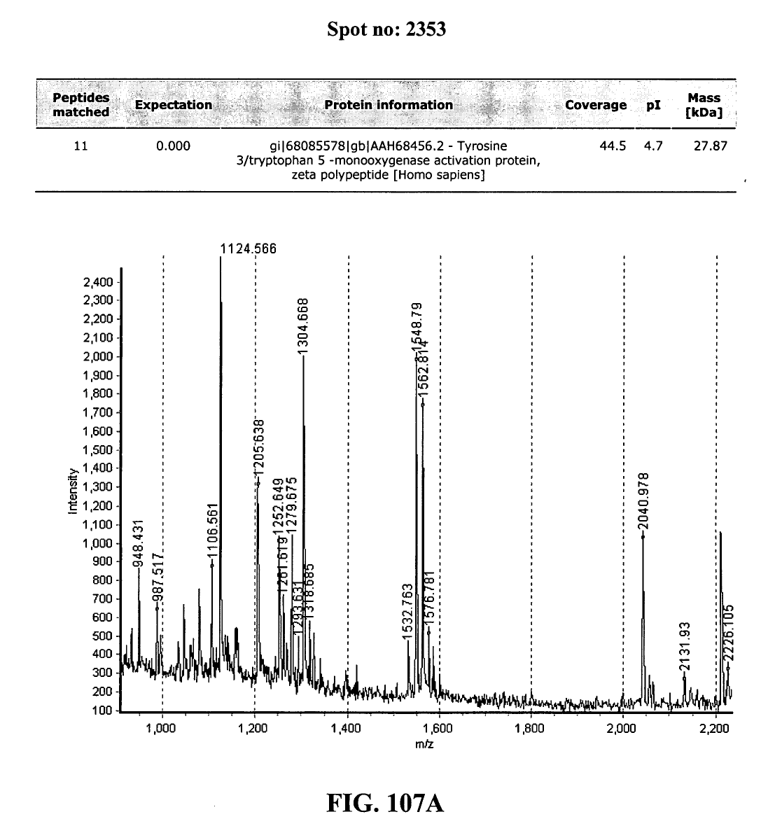

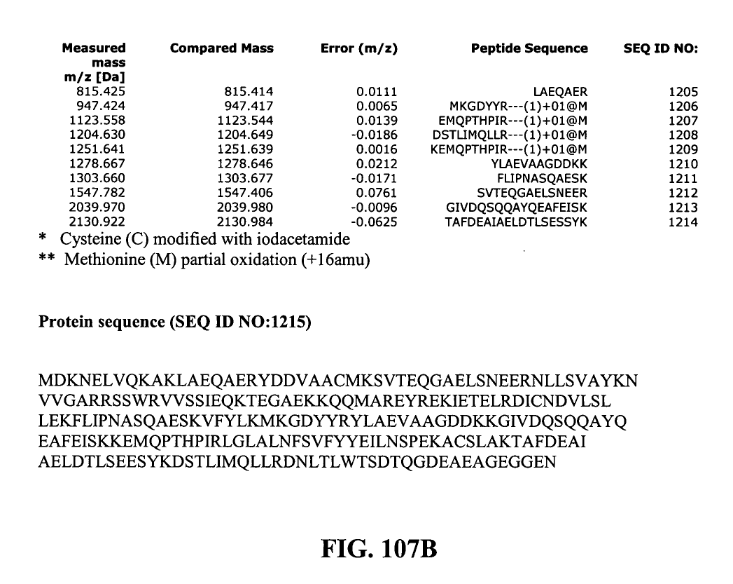

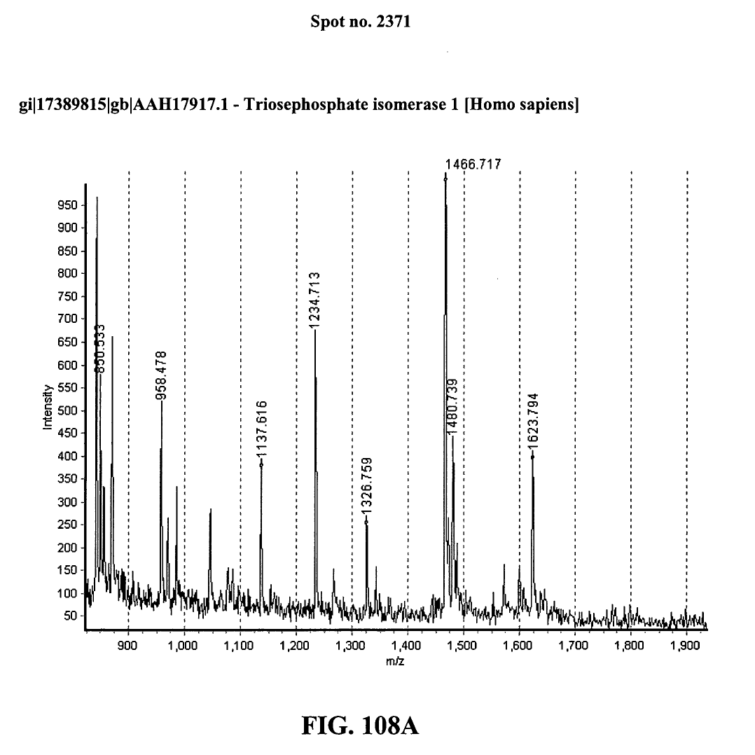

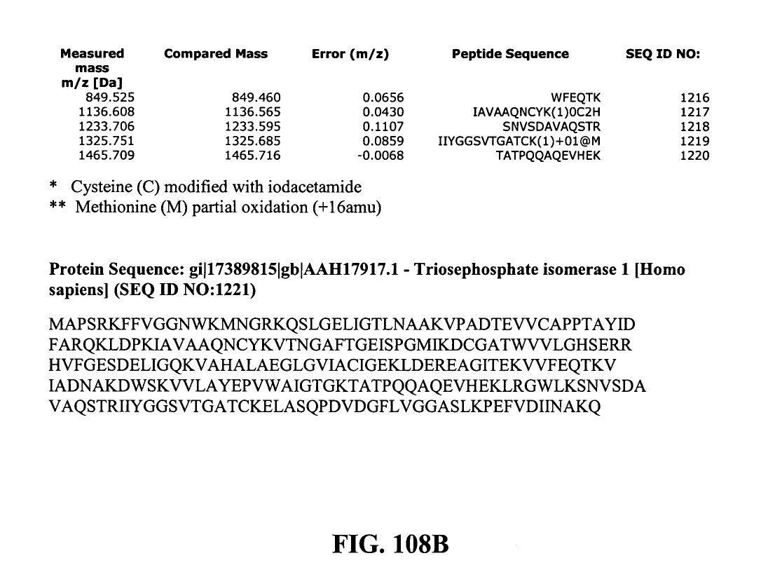

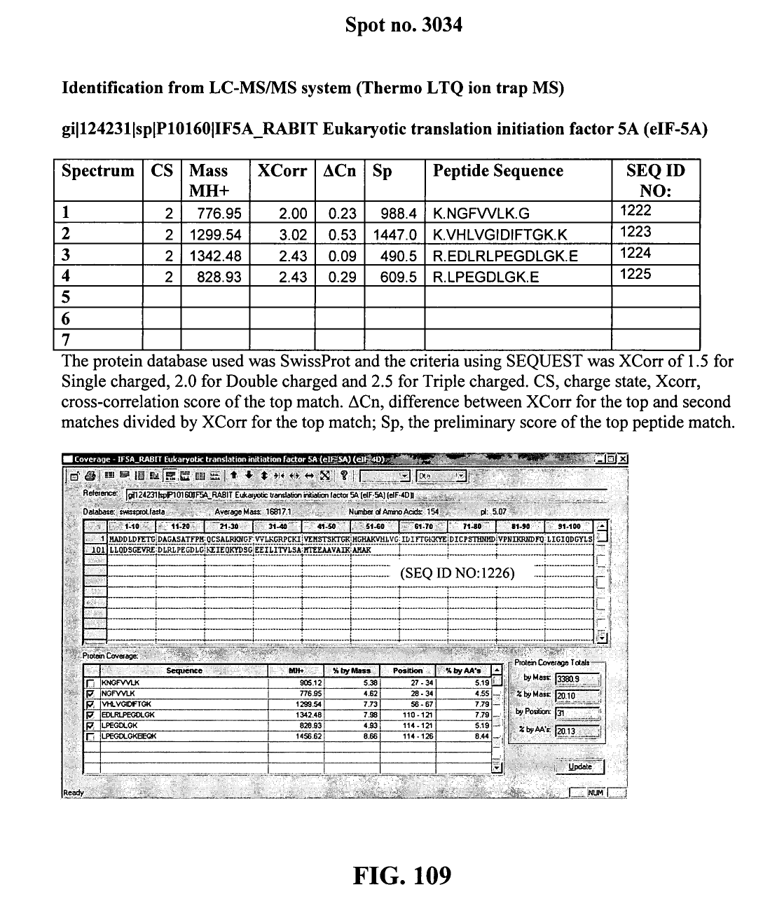

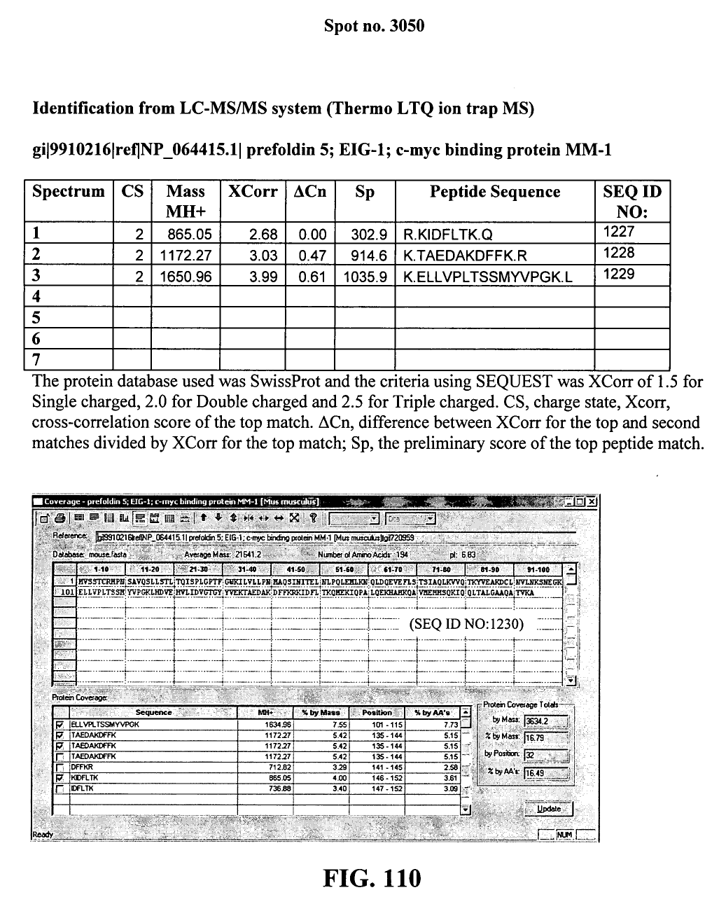

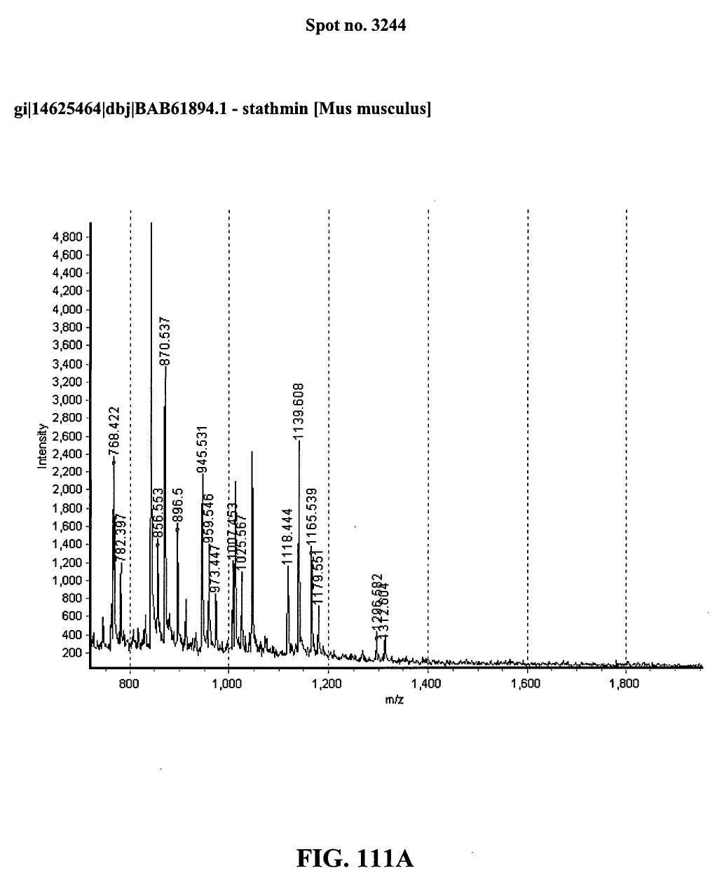

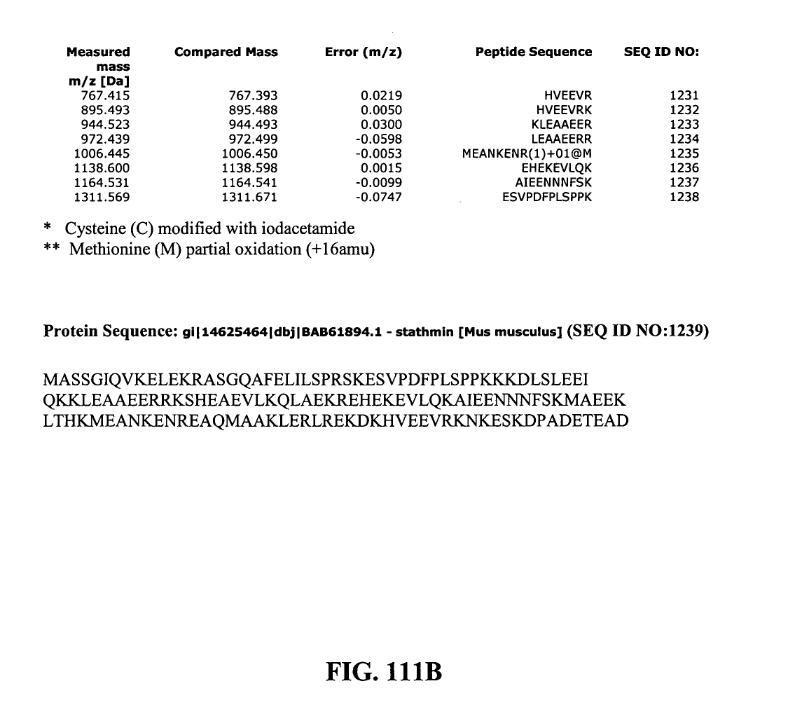

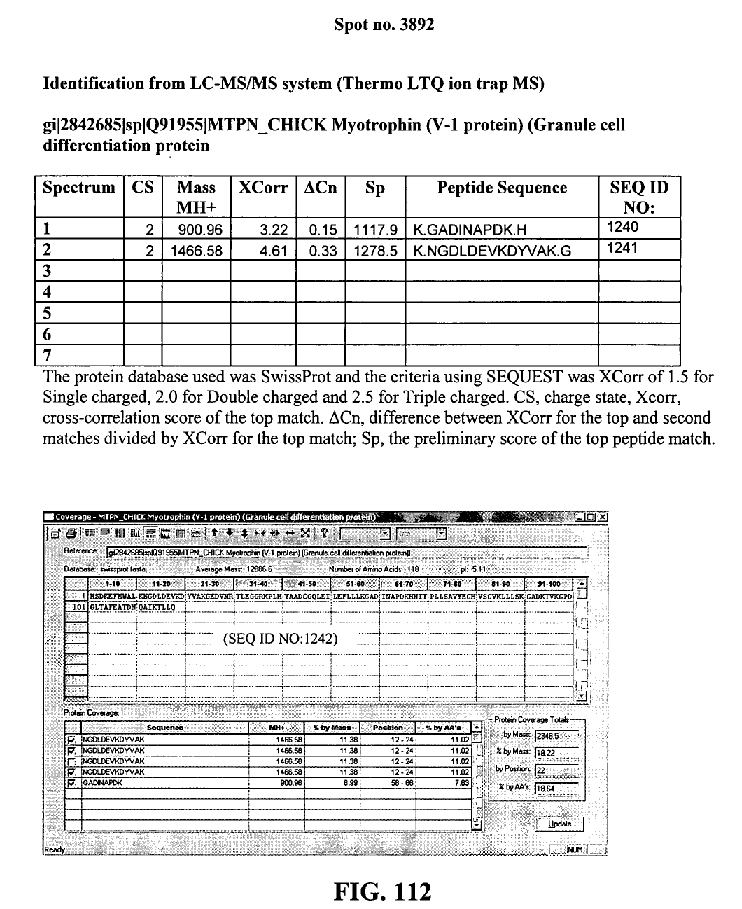

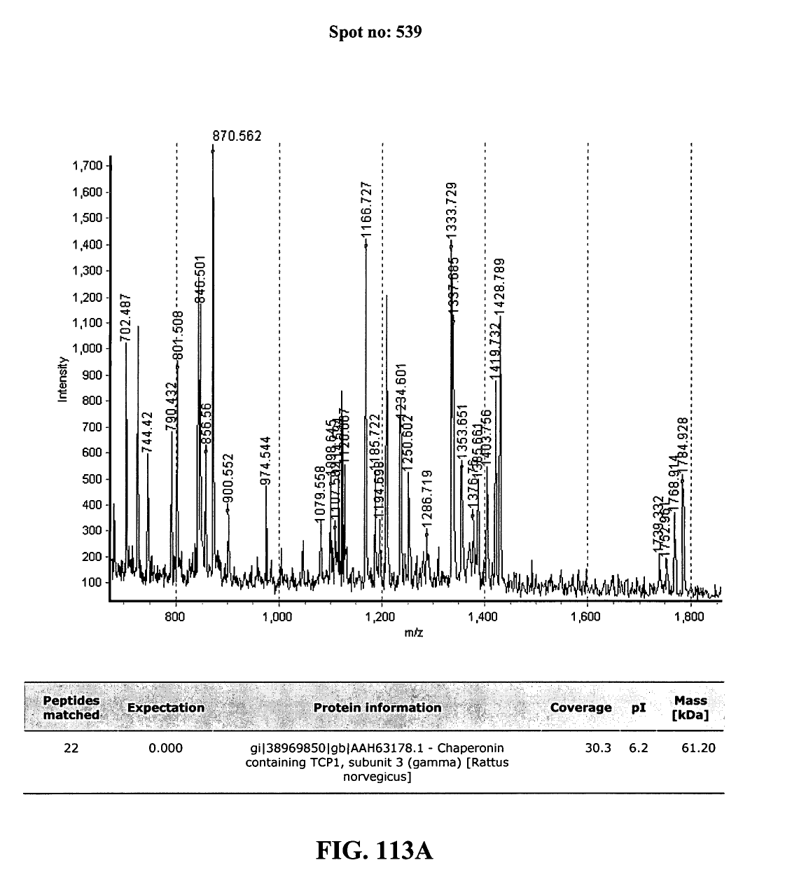

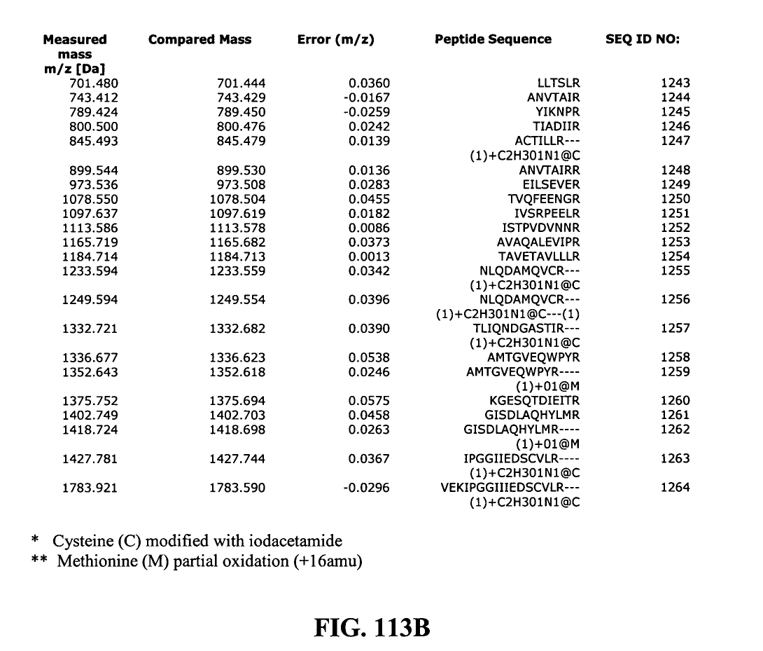

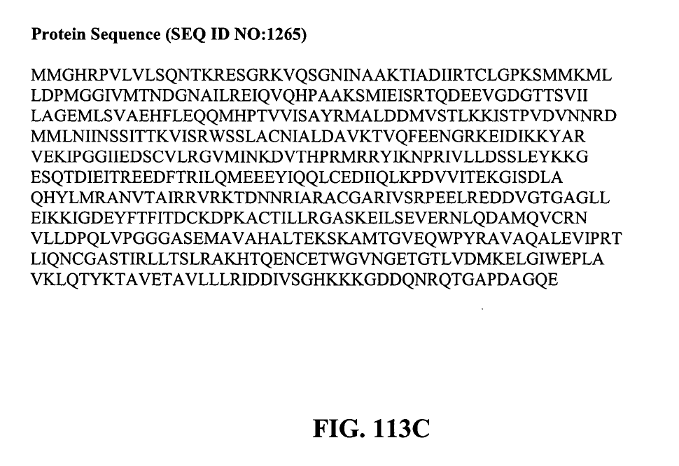

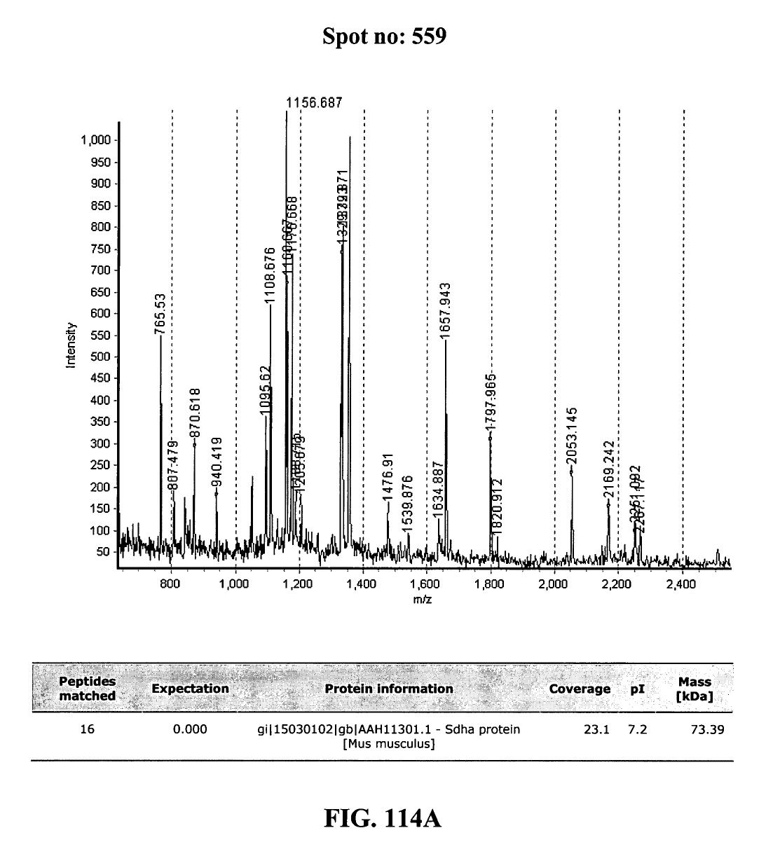

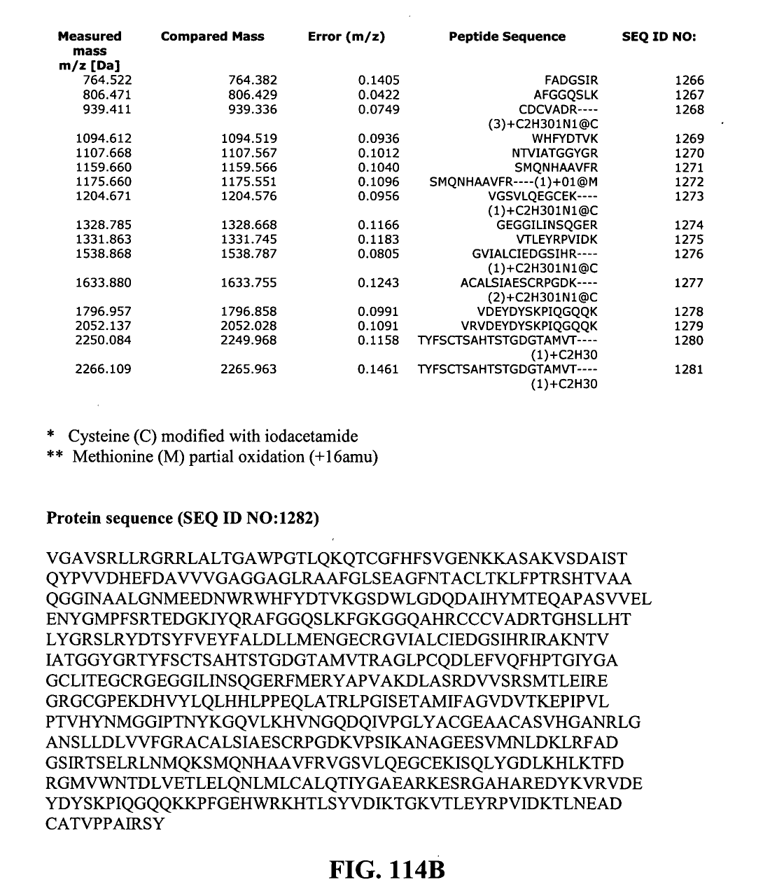

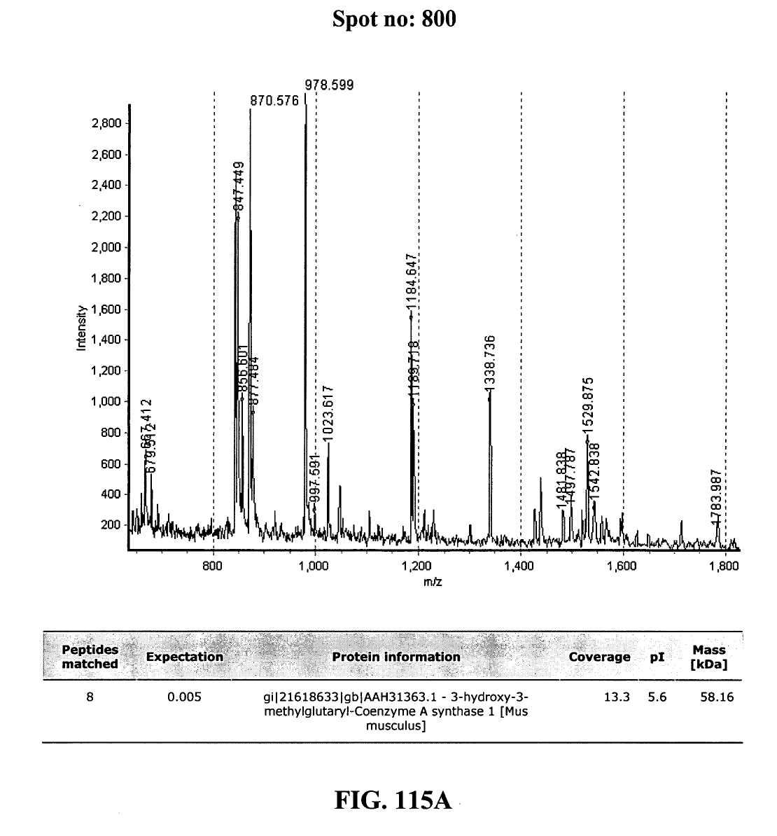

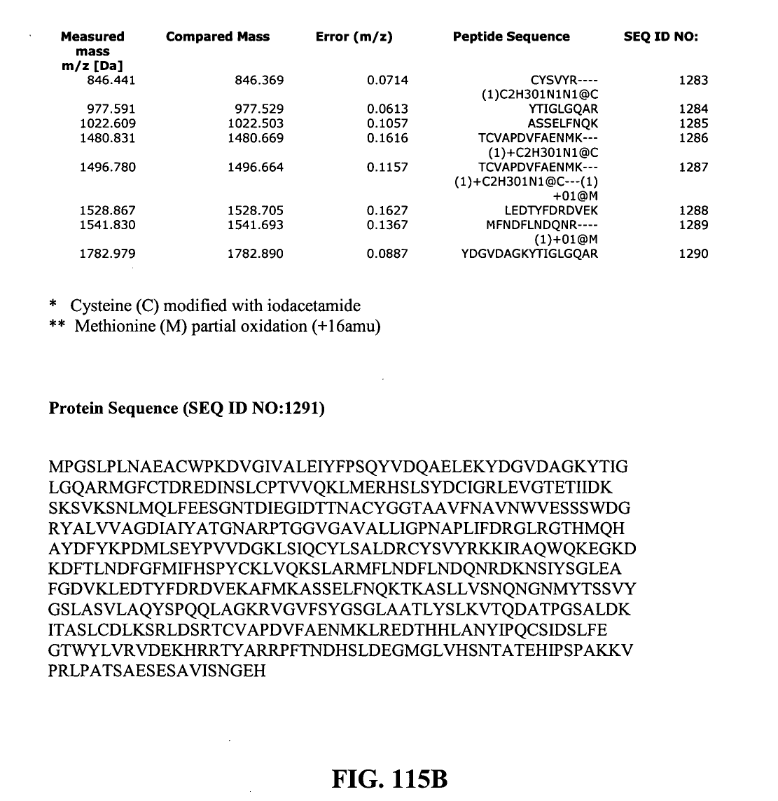

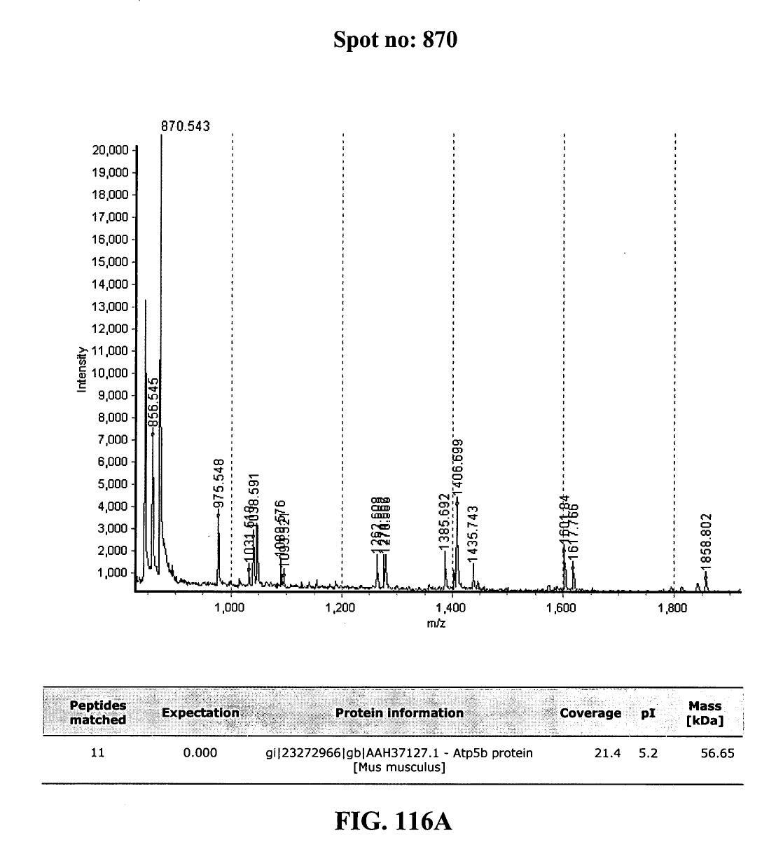

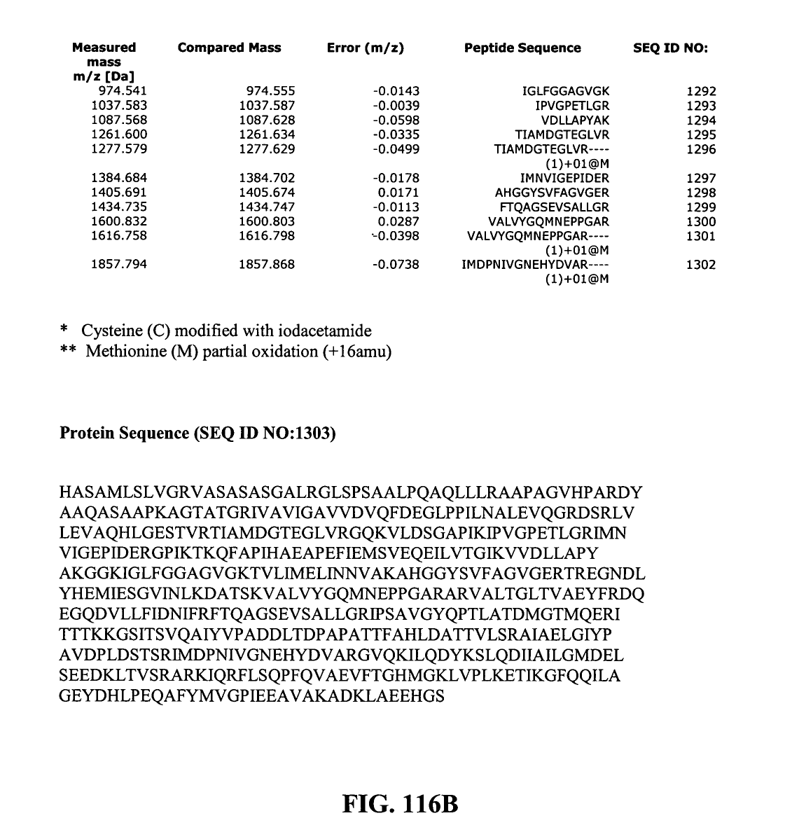

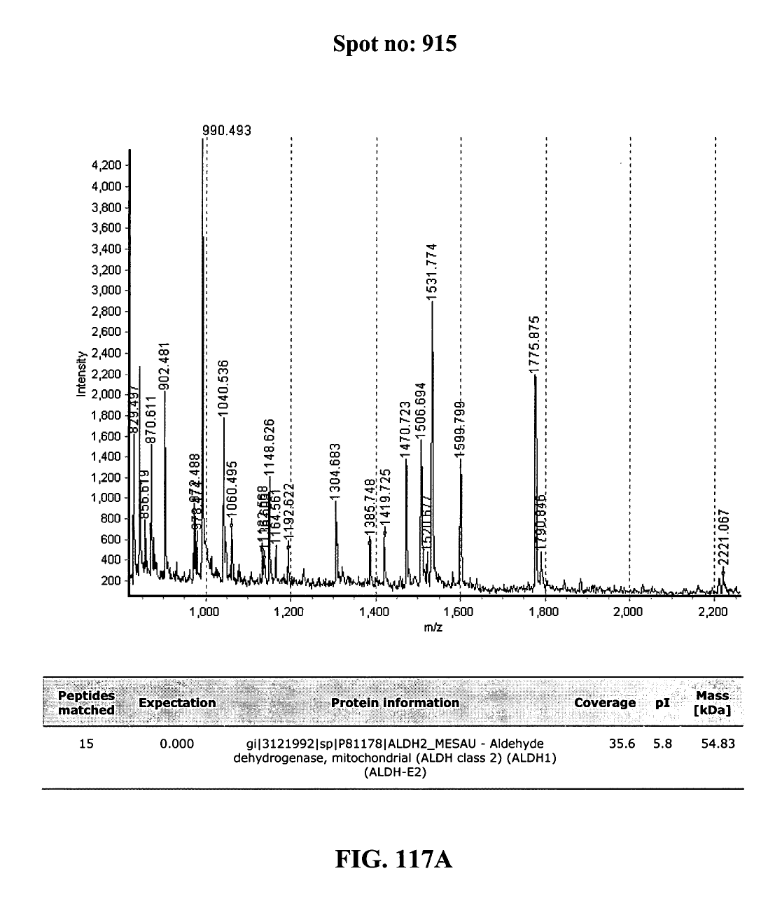

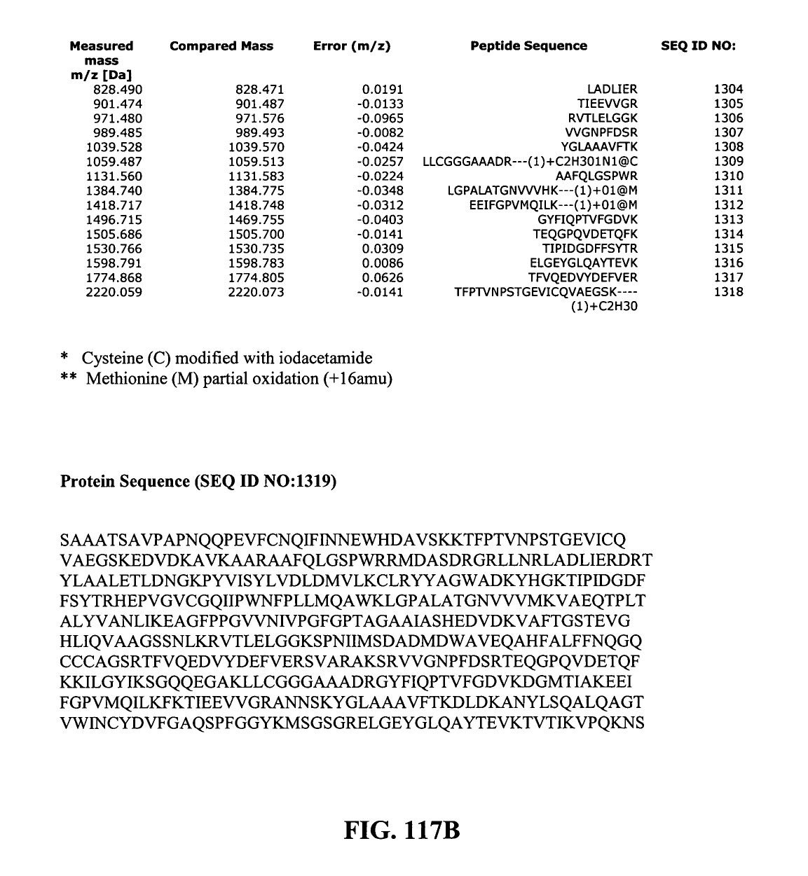

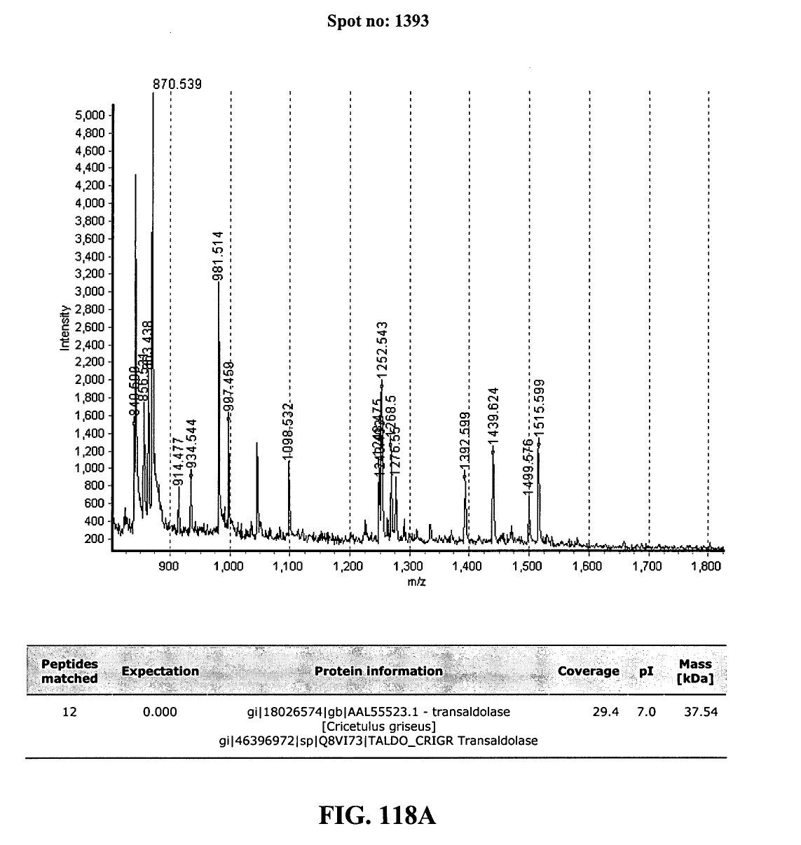

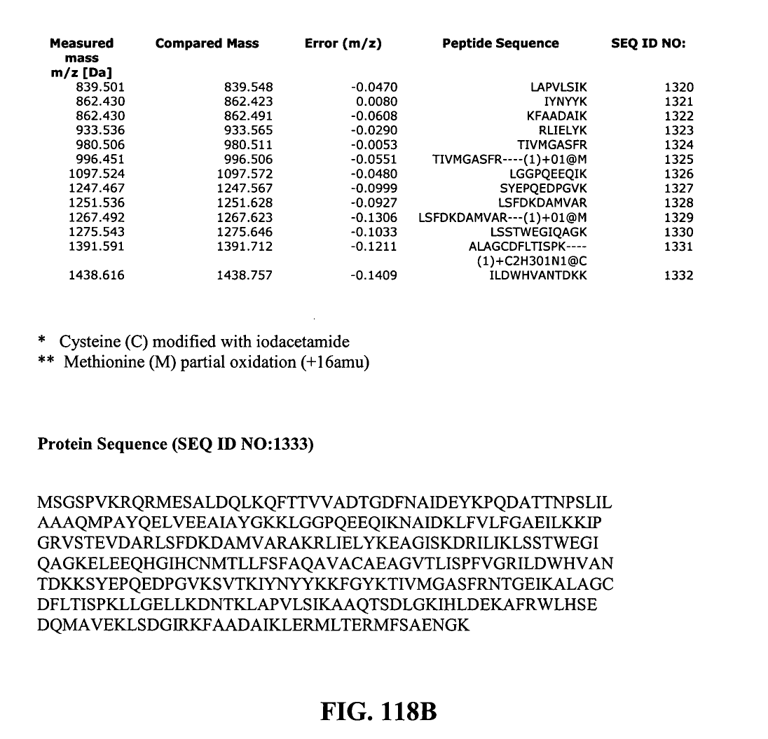

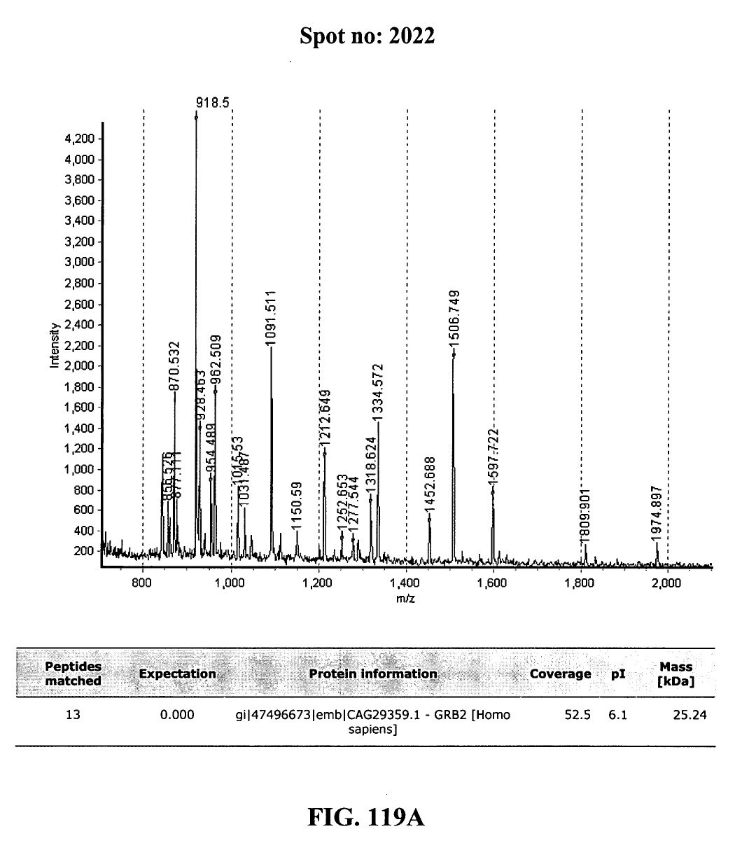

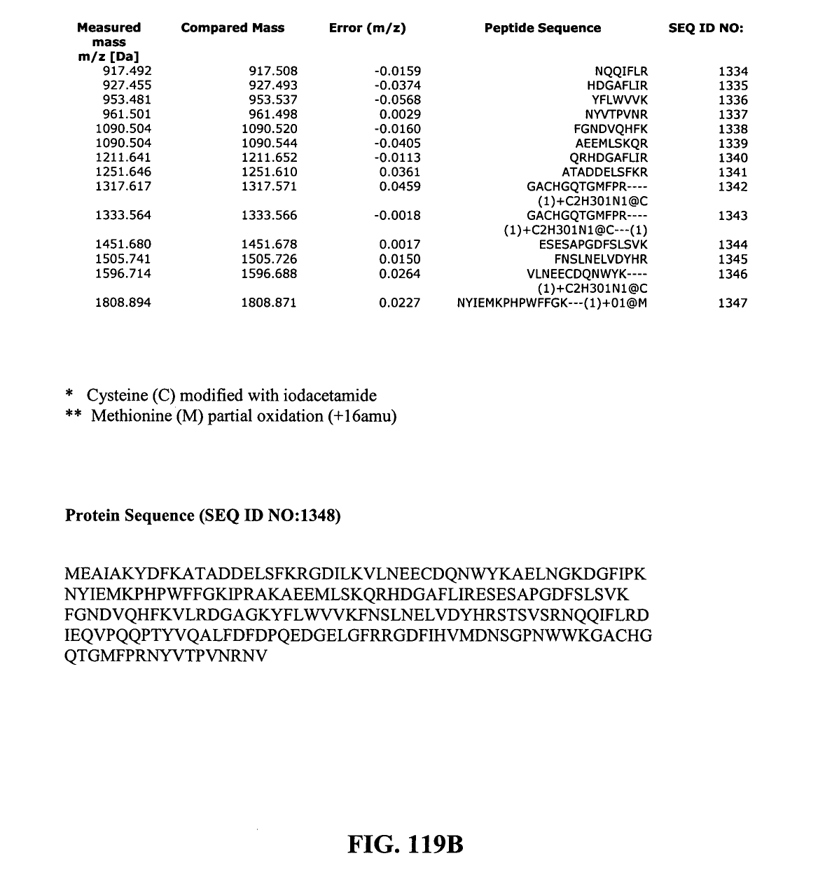

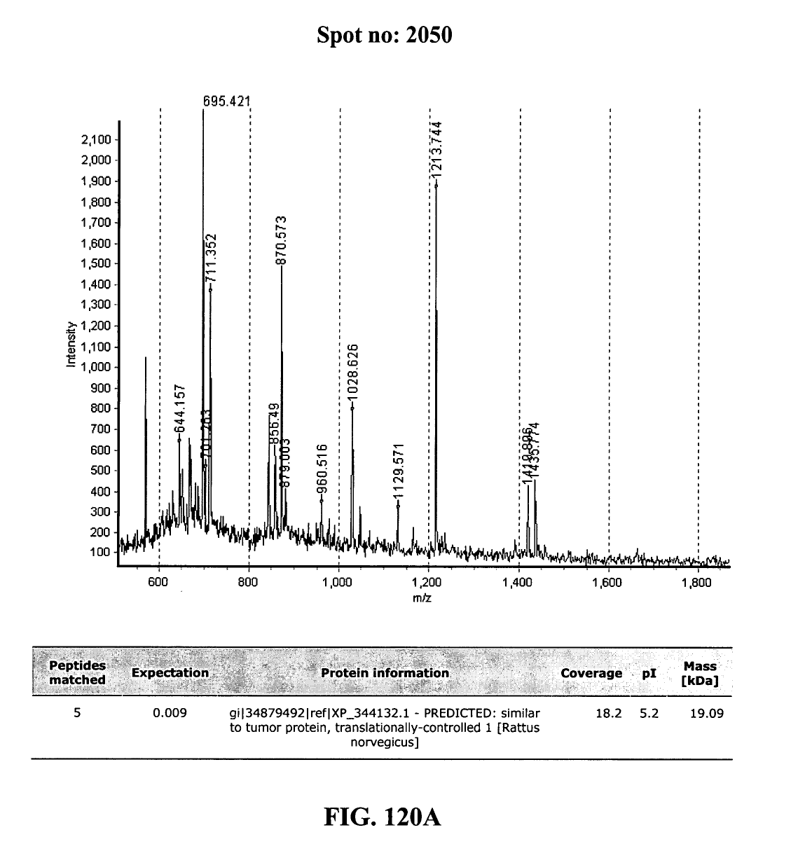

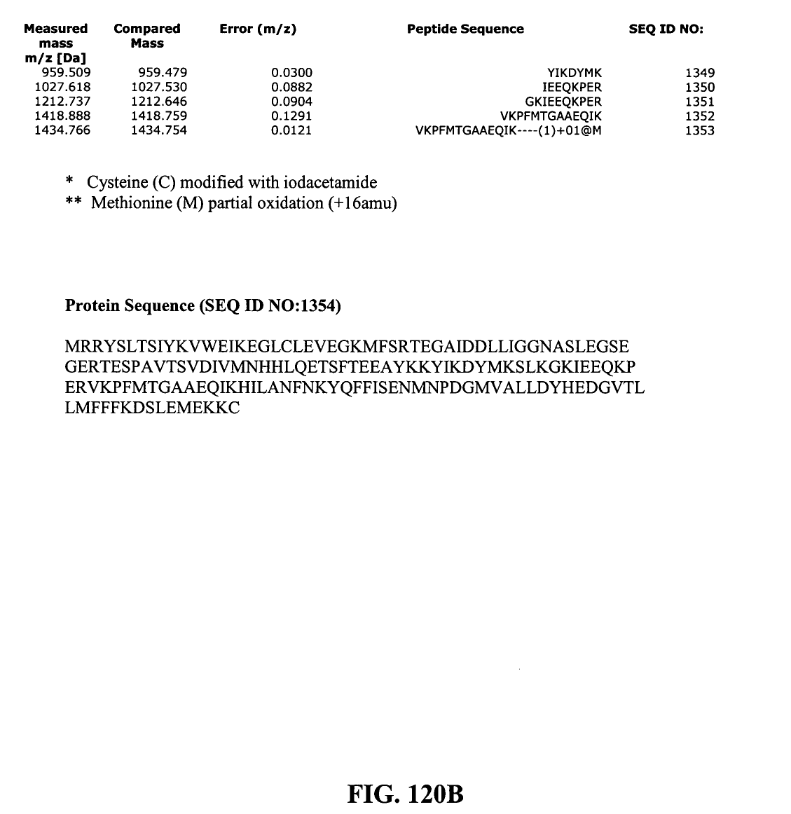

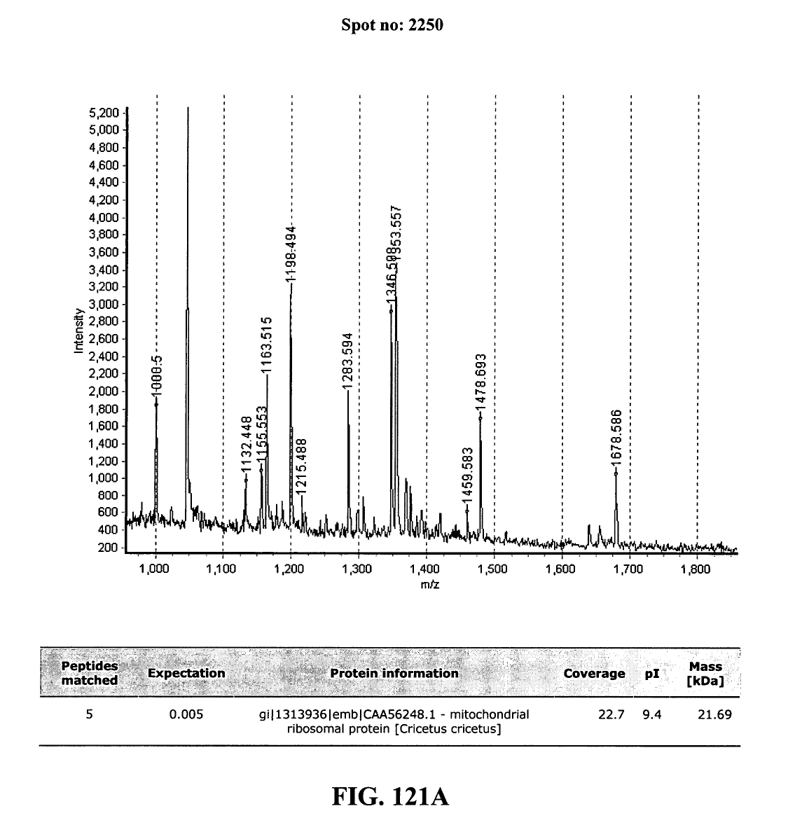

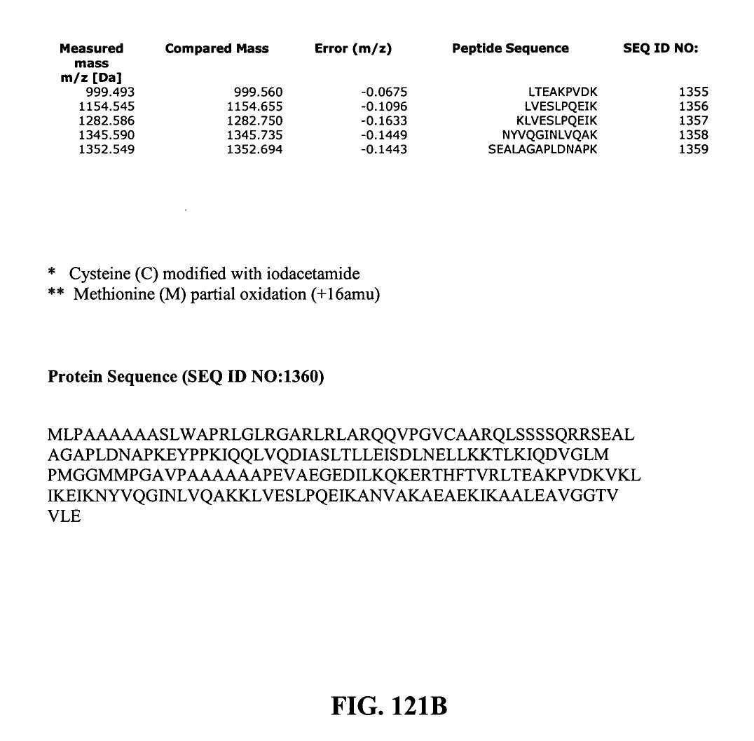

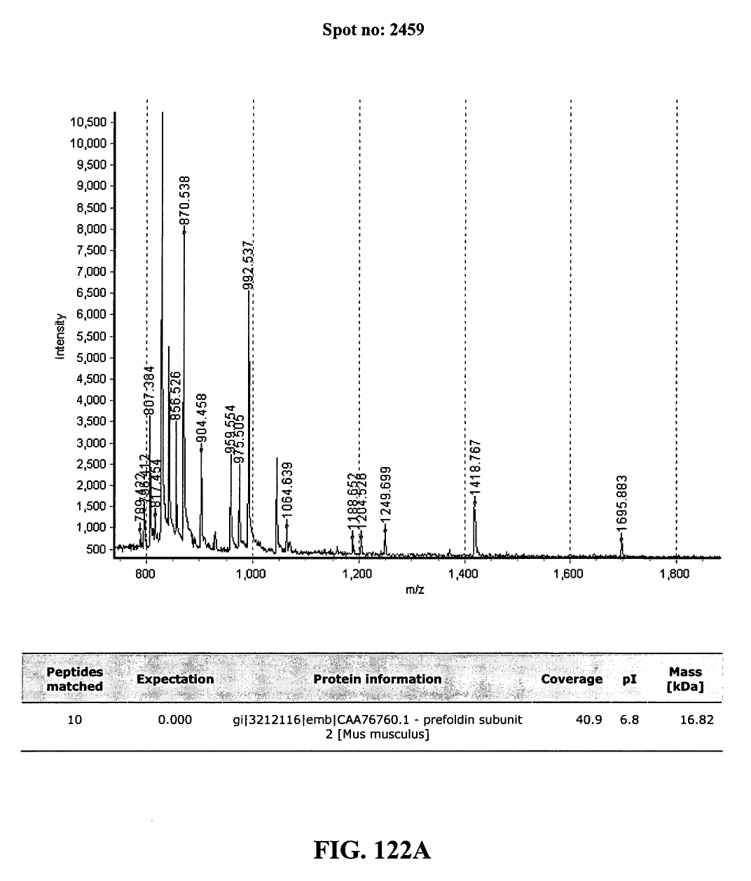

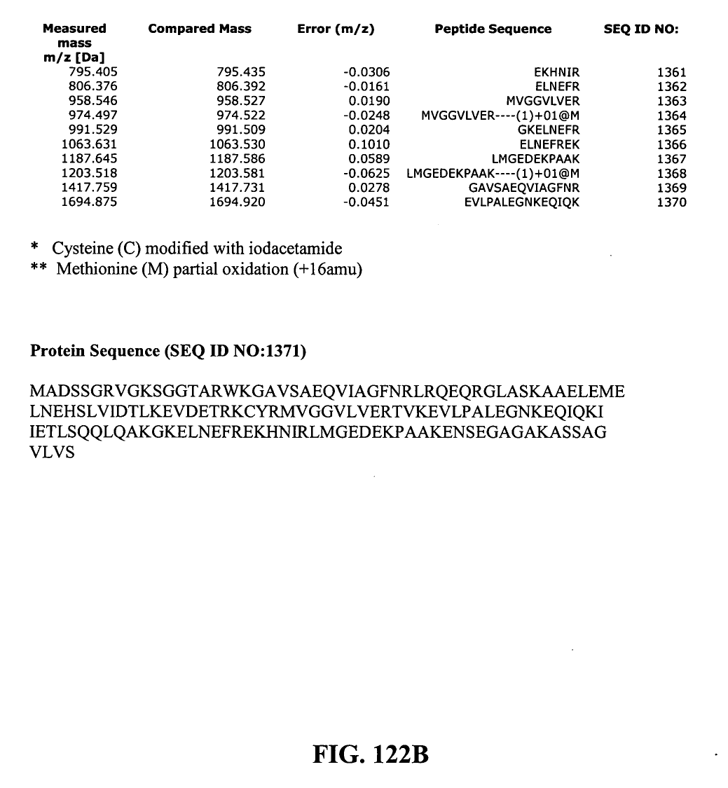

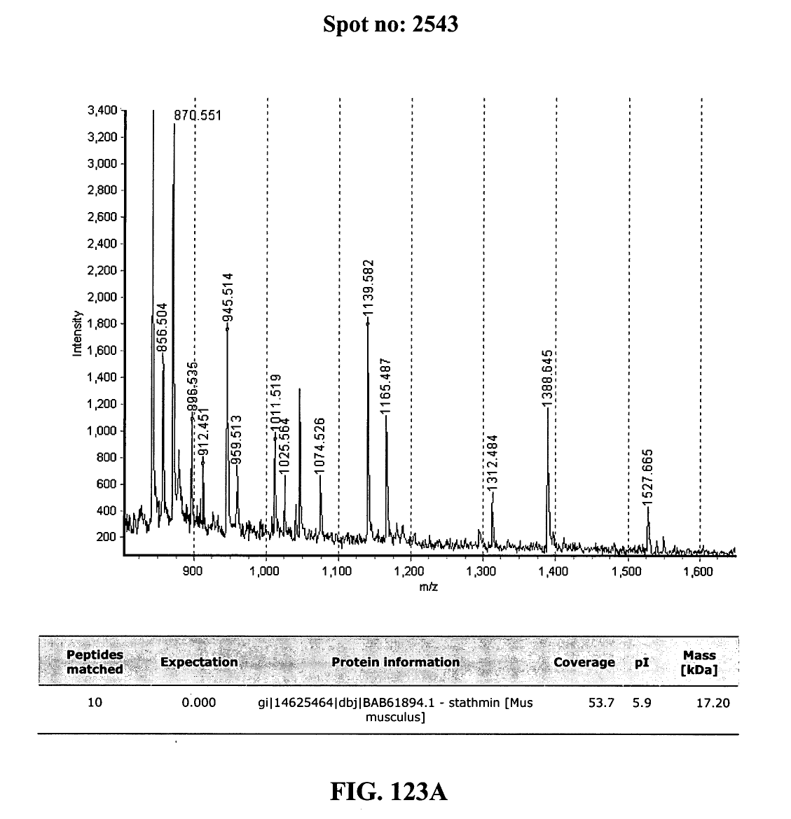

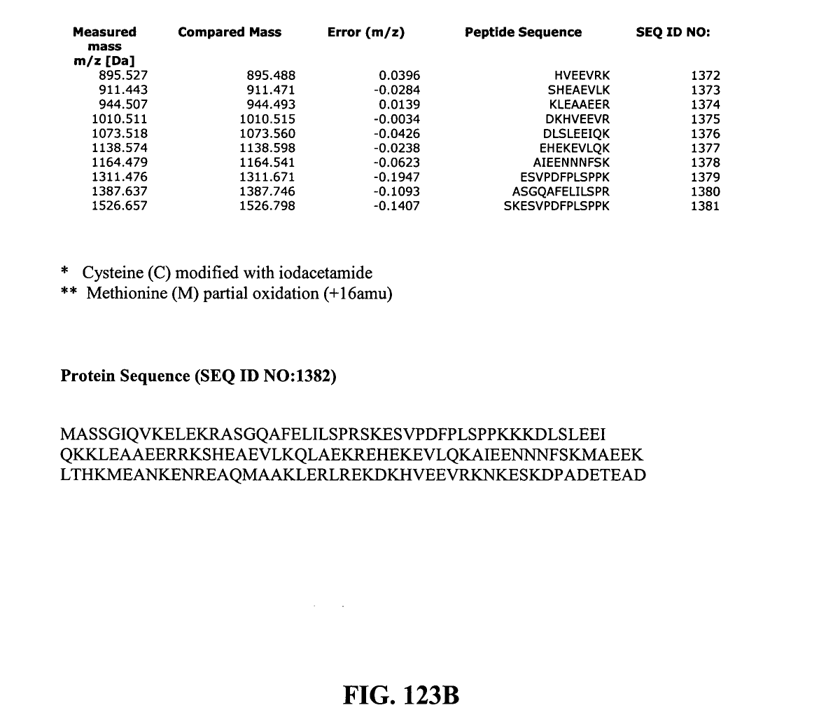

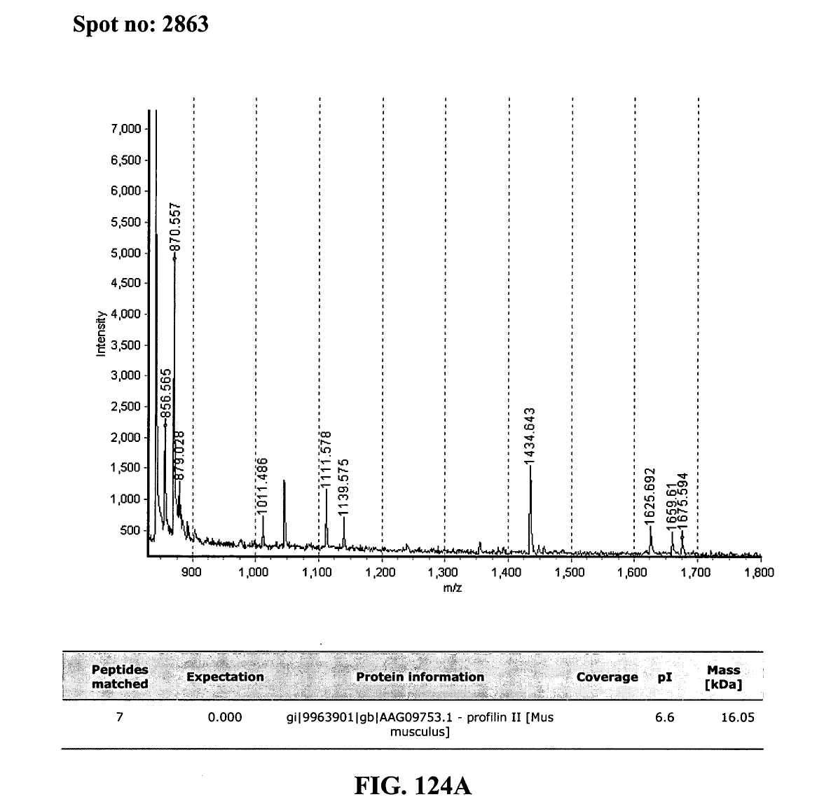

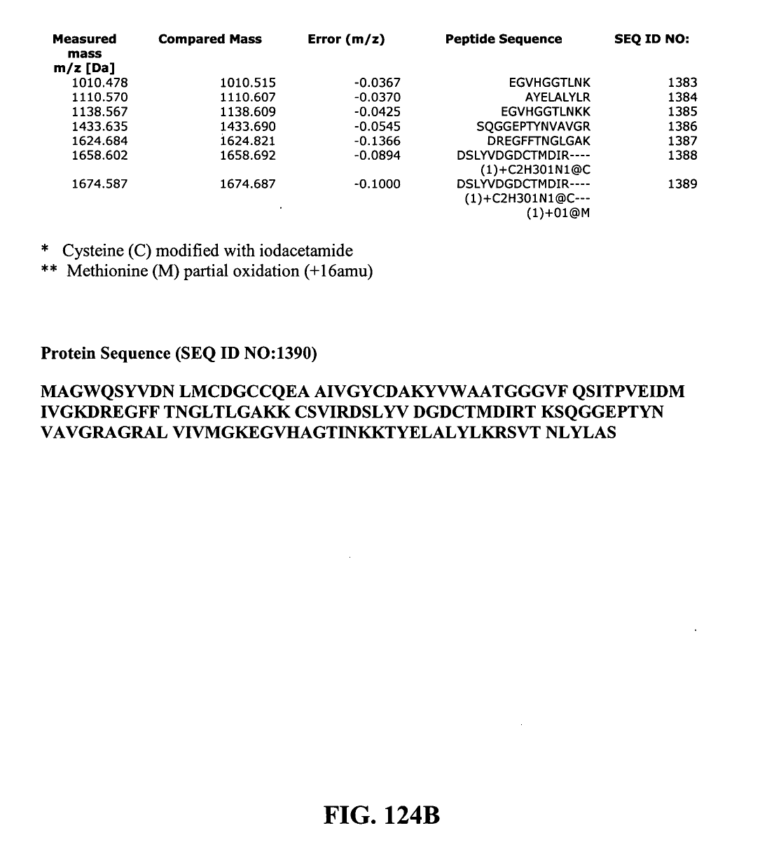

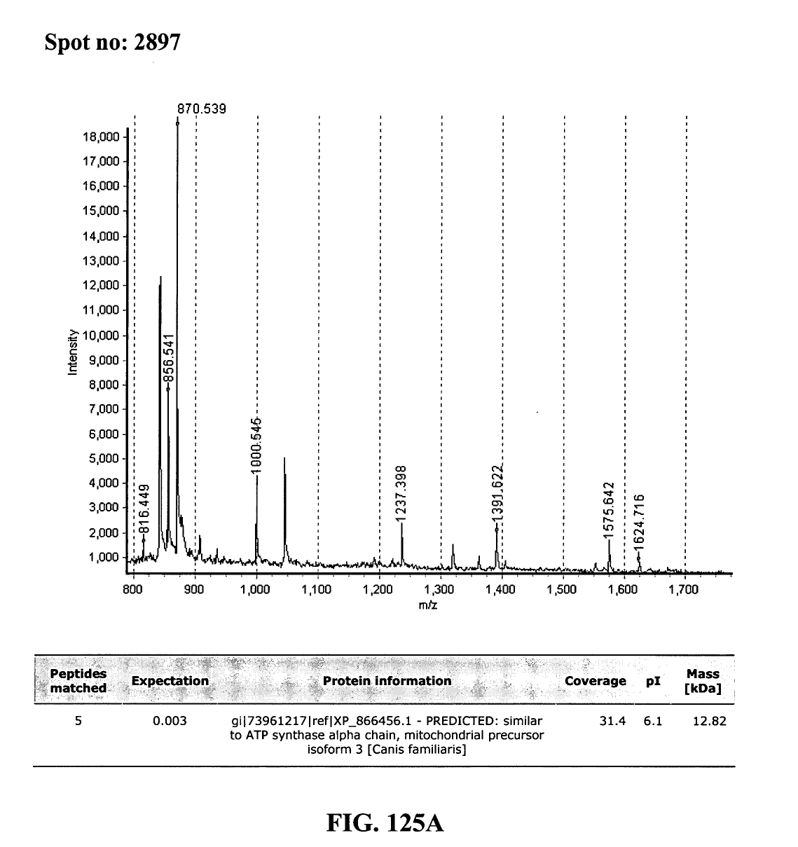

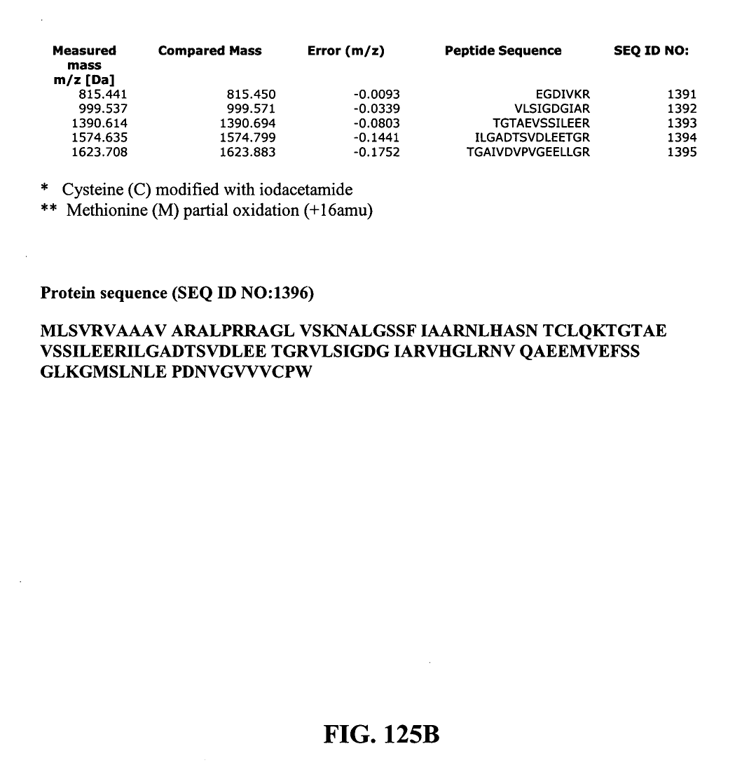

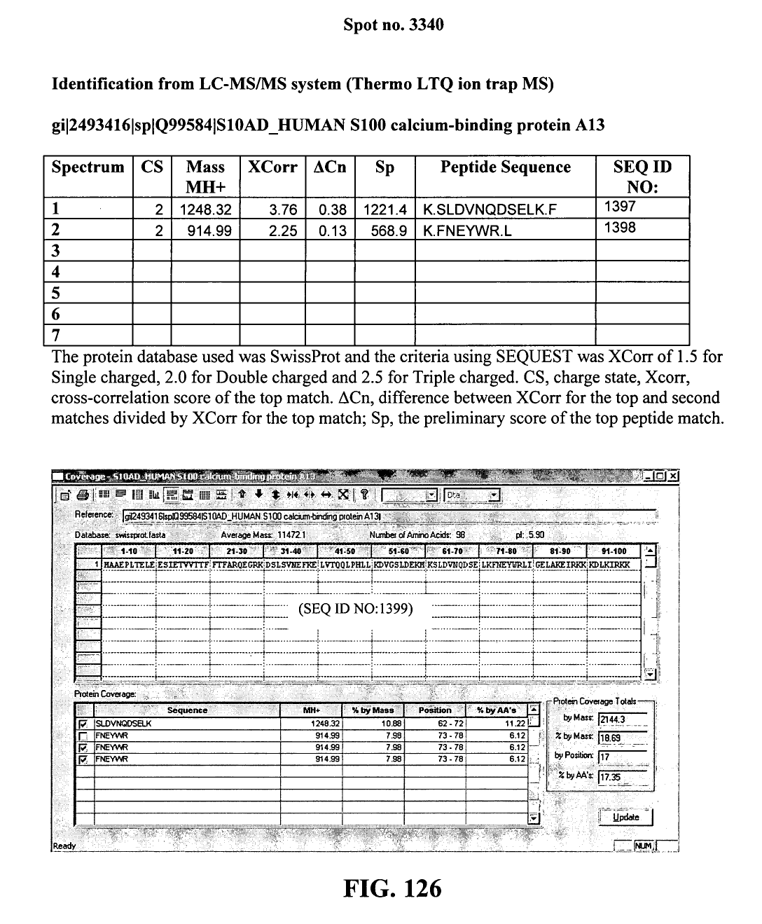

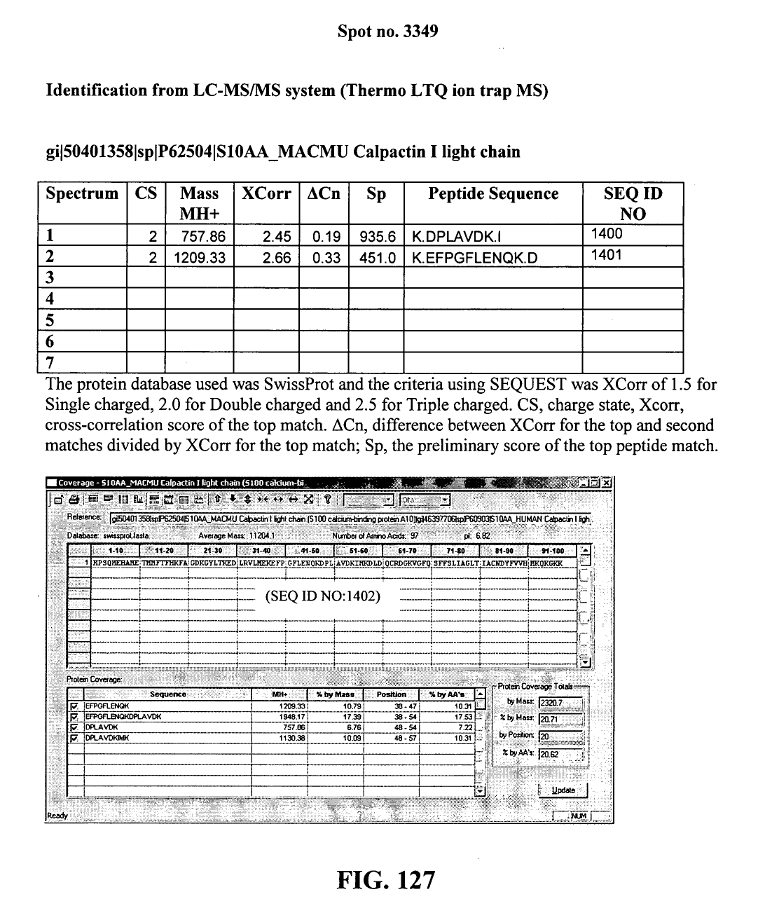

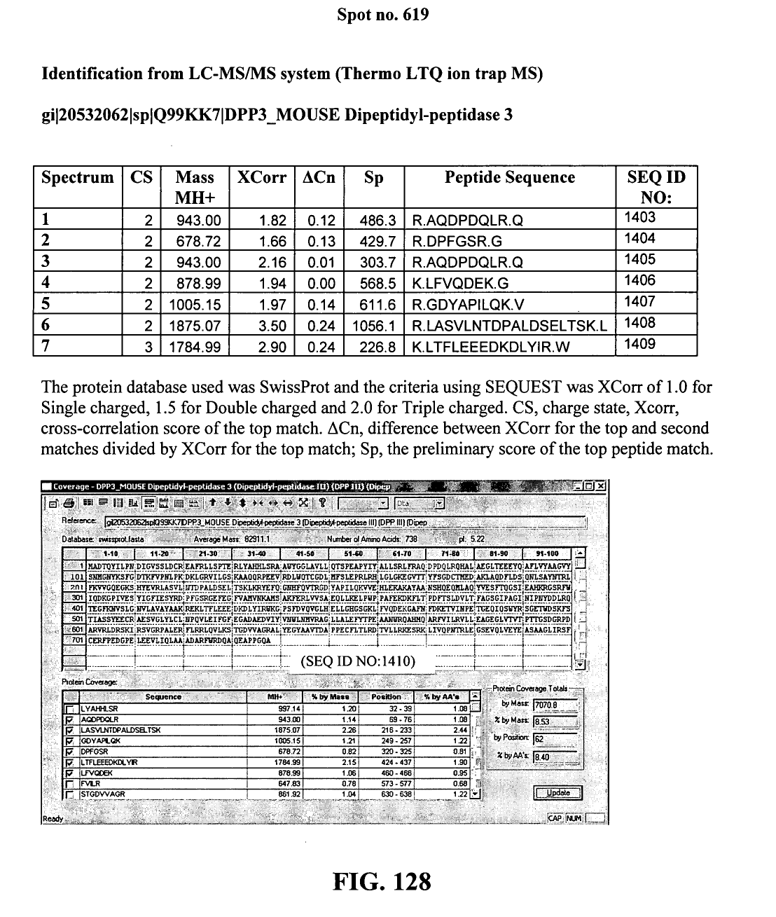

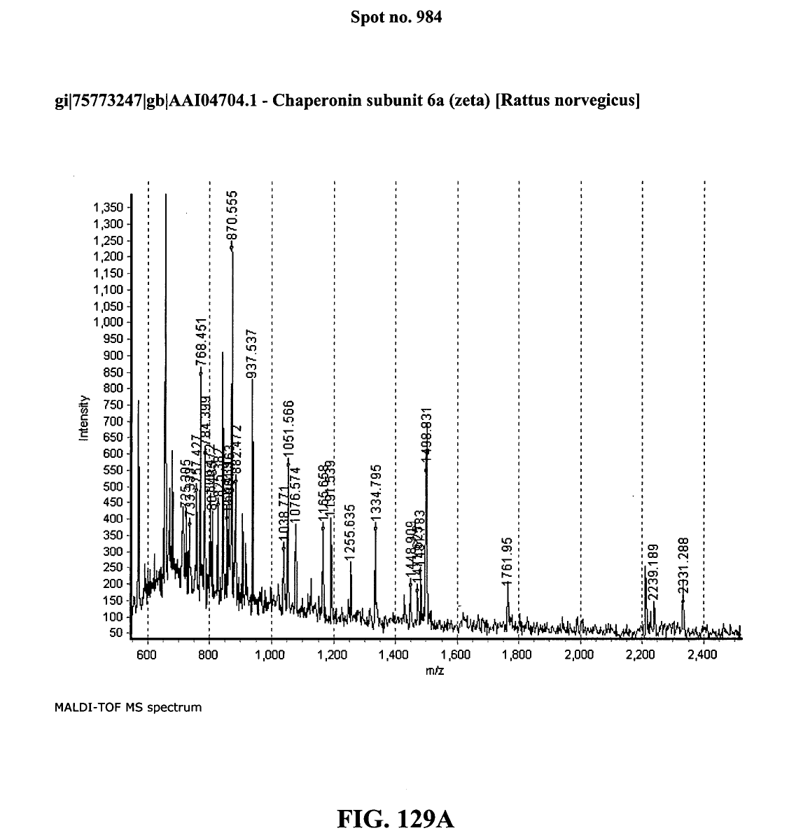

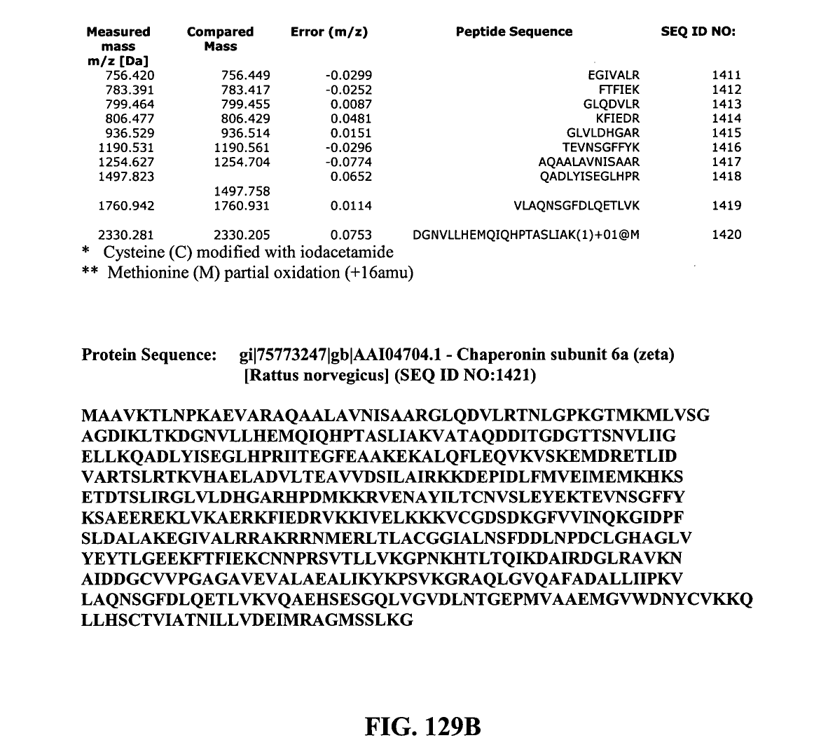

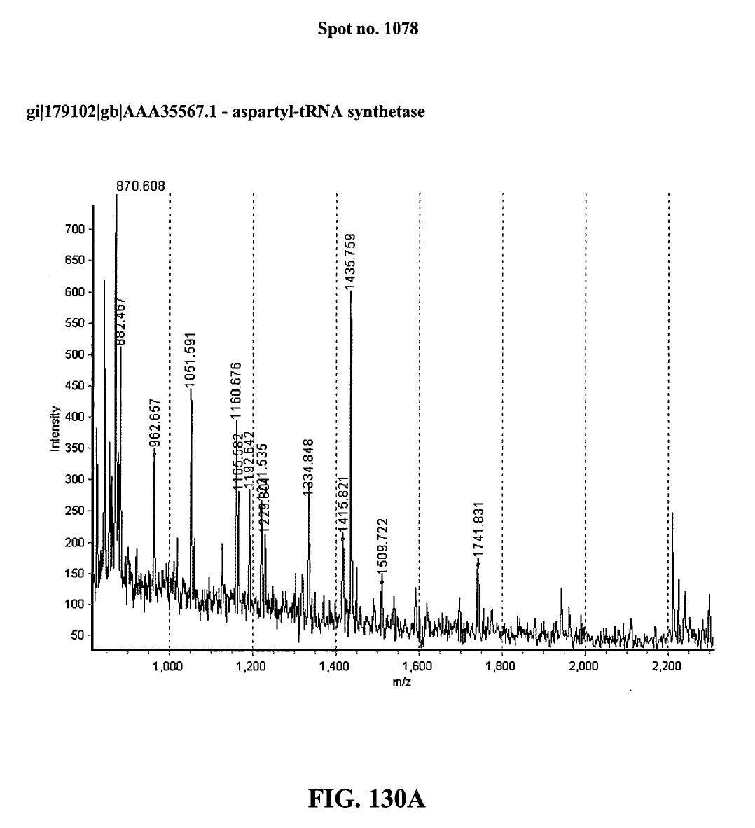

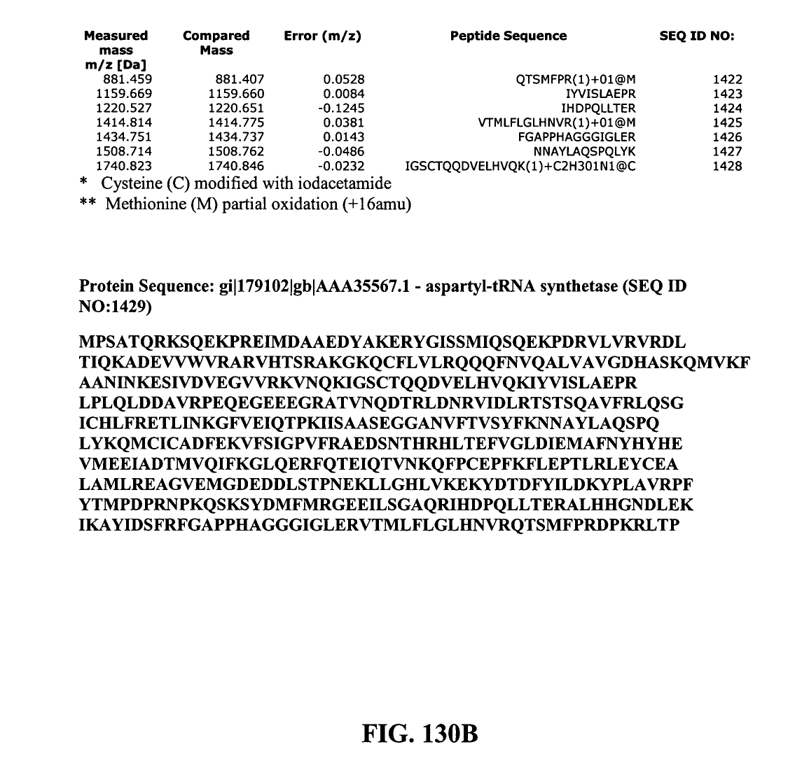

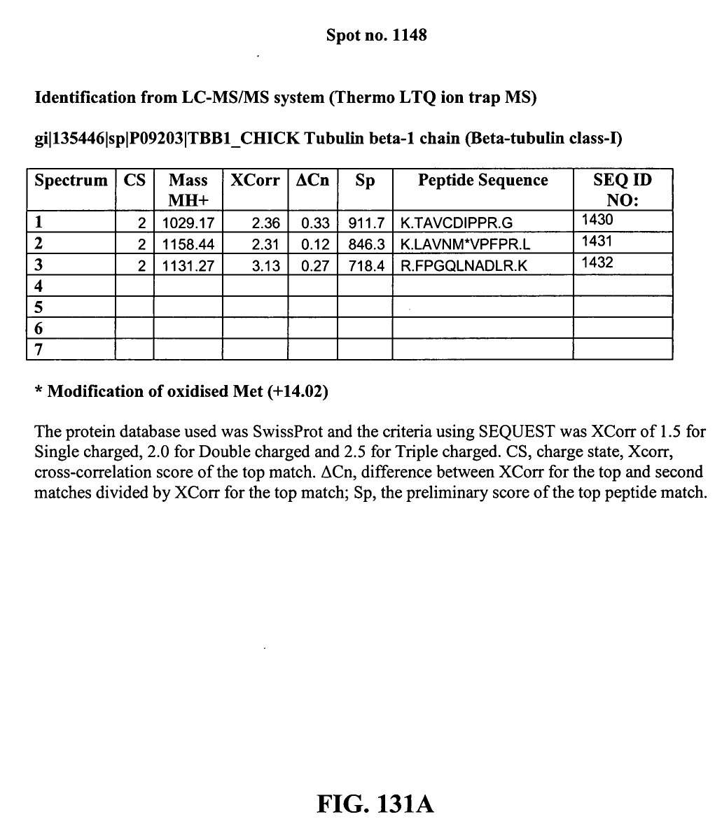

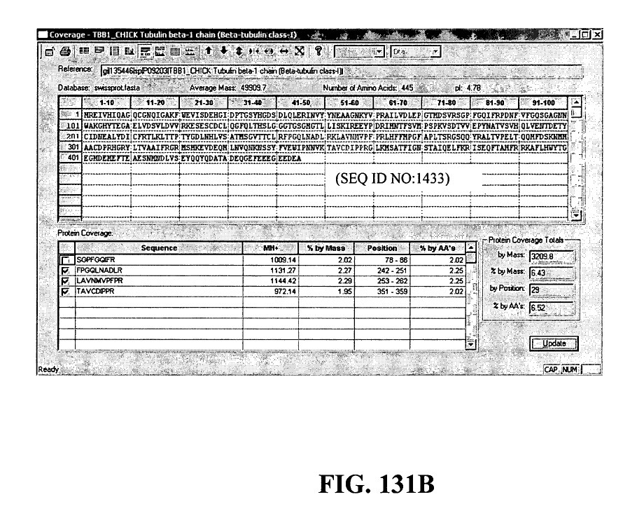

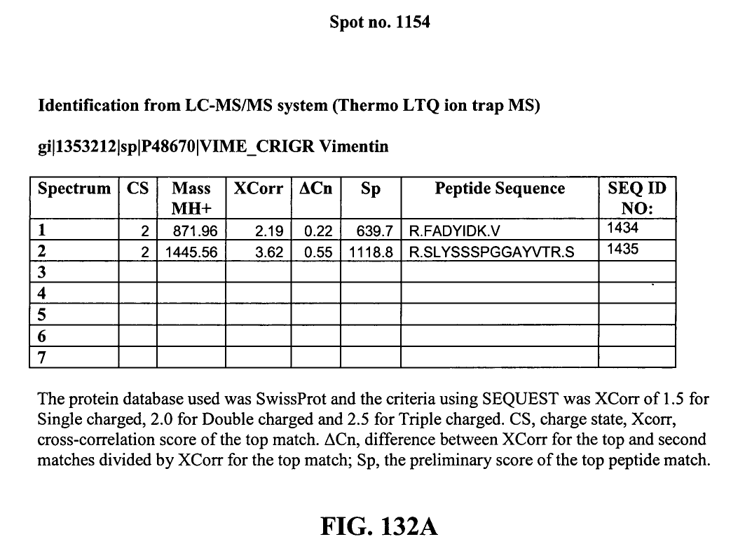

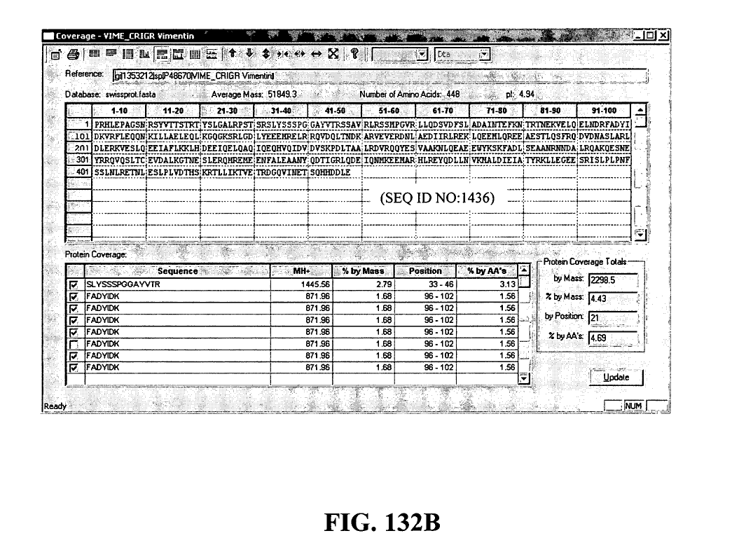

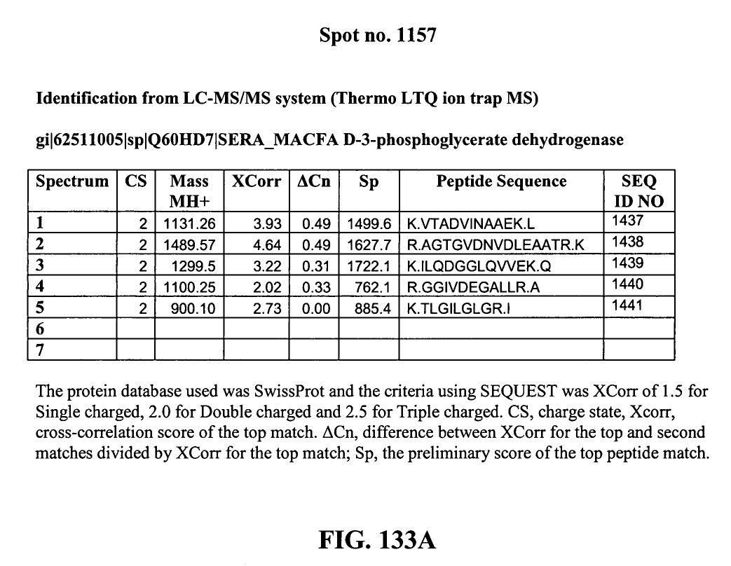

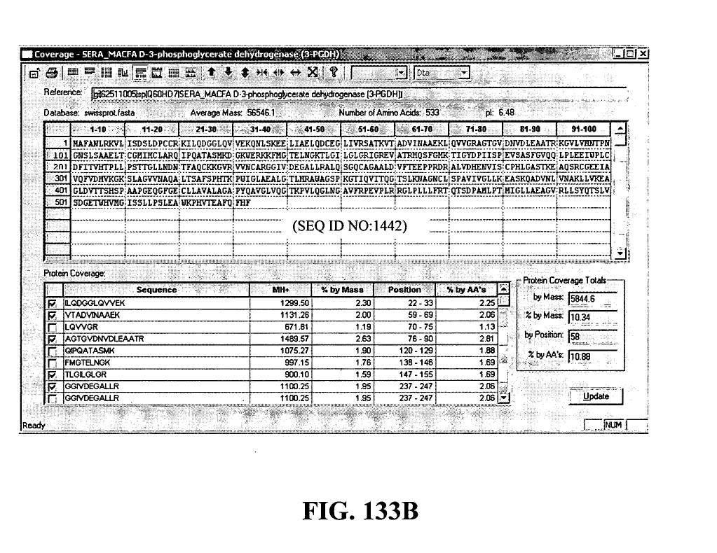

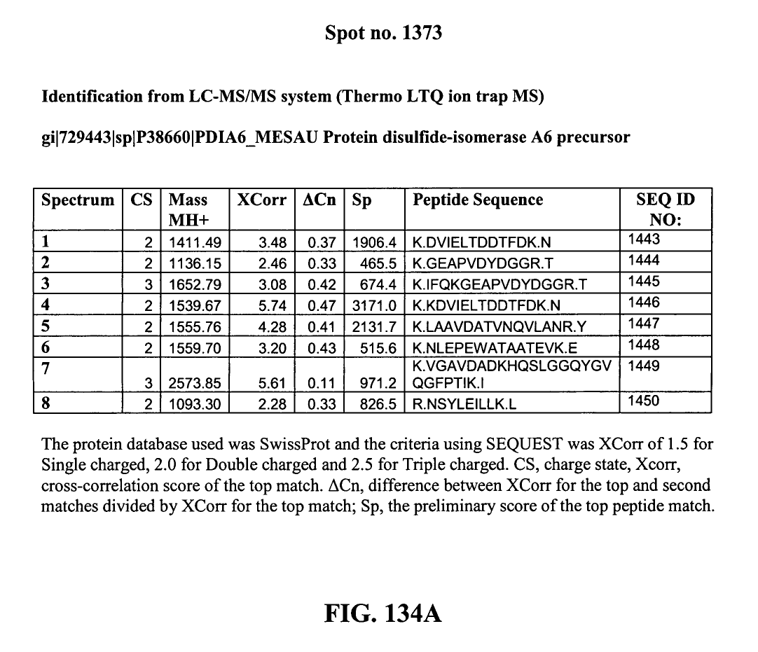

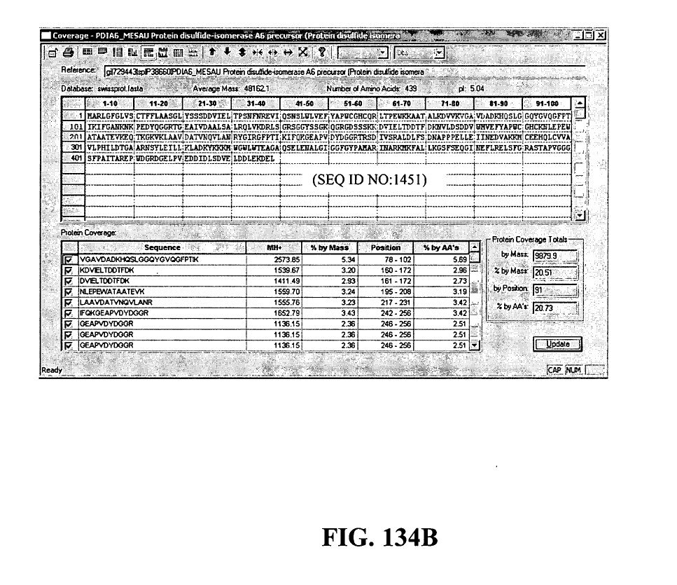

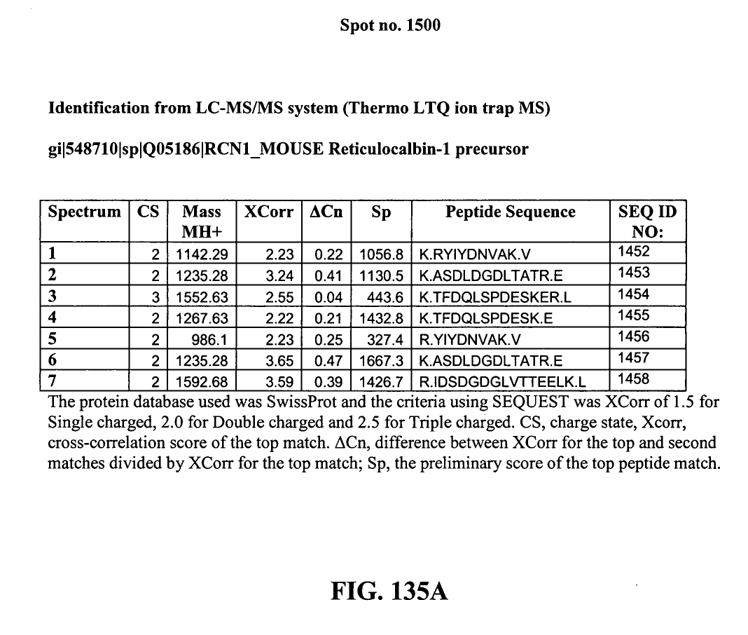

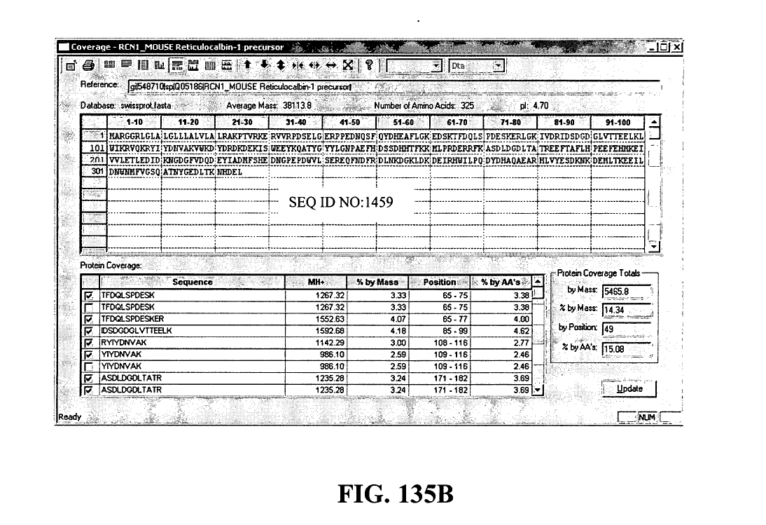

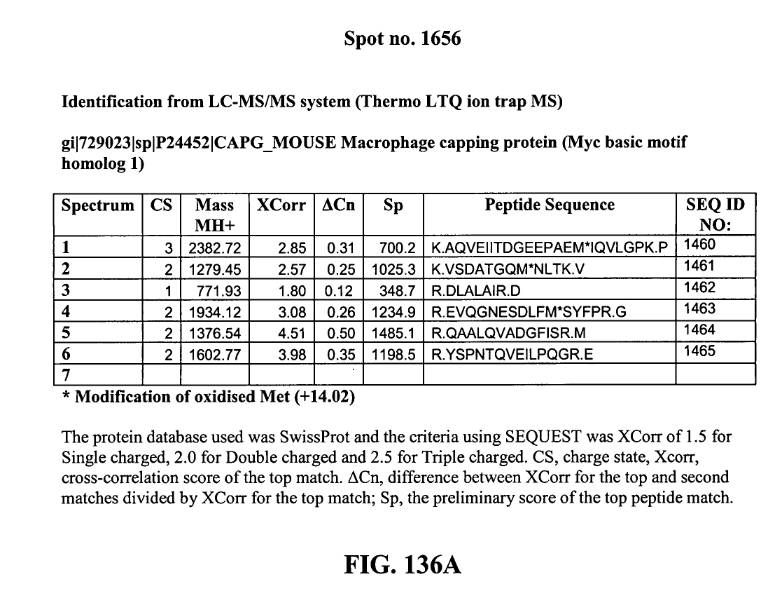

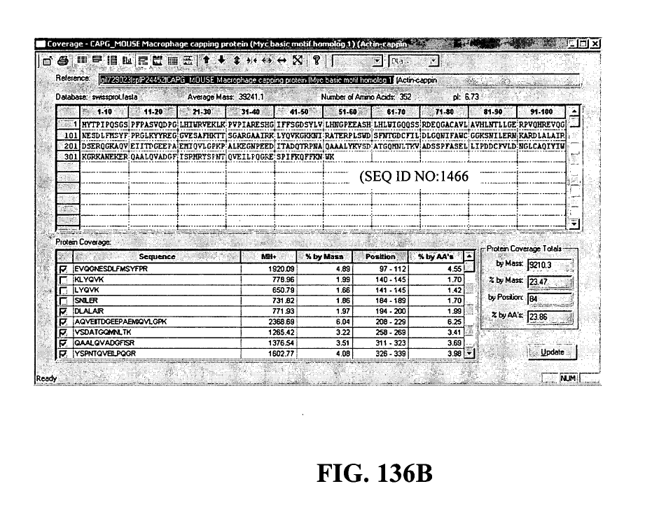

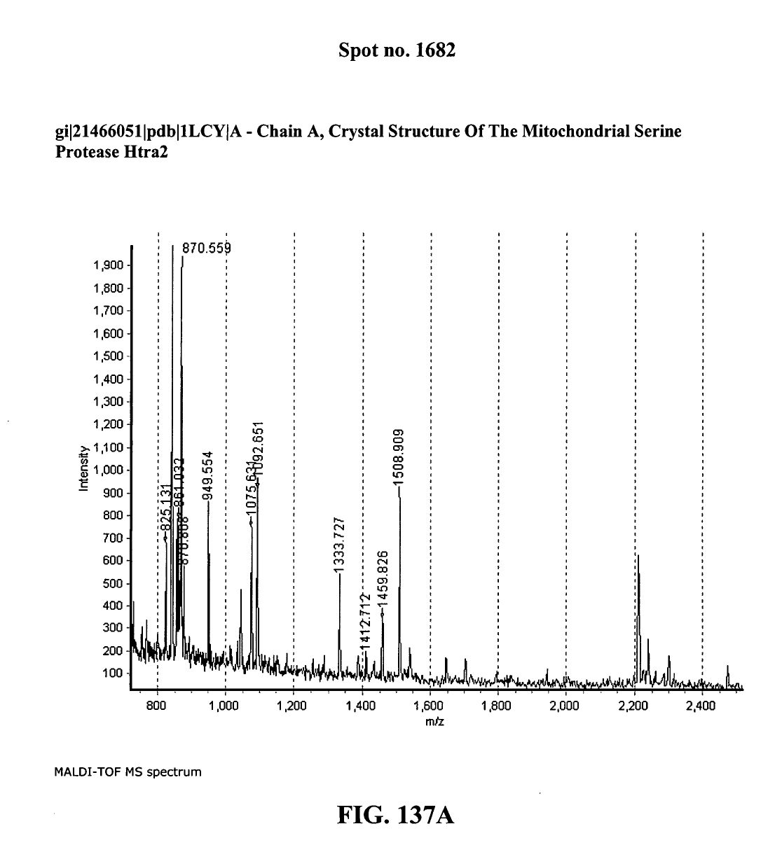

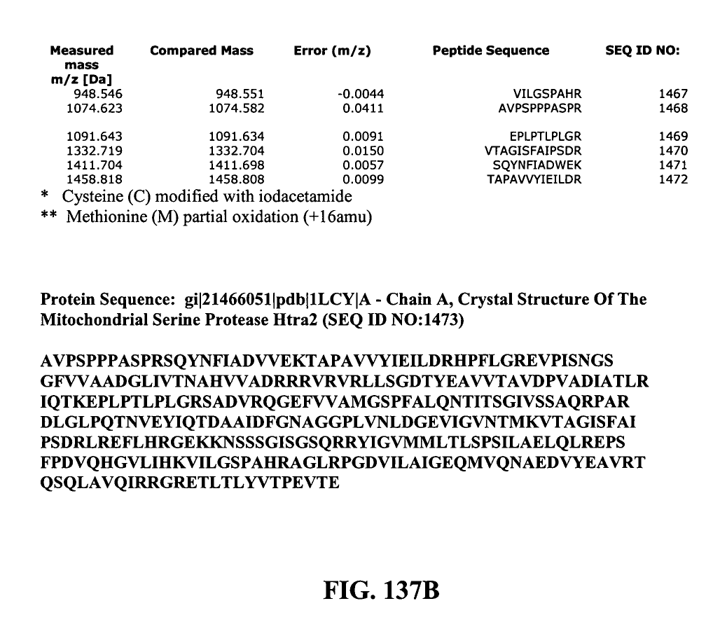

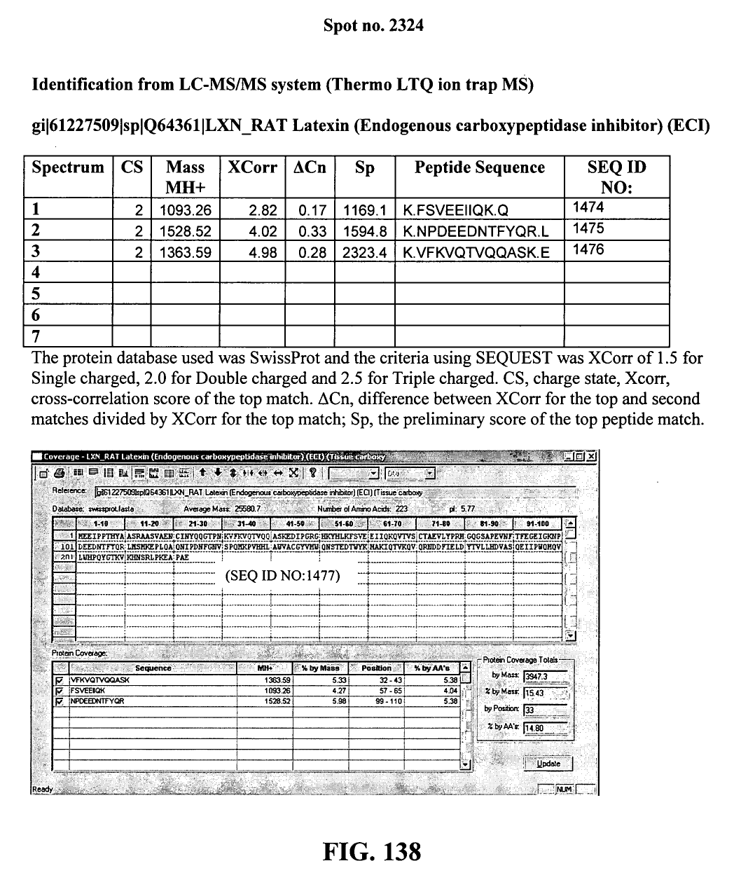

28. A method of assessing a cell culture phenotype of a cell line, the method comprising detecting, in a sample from the cell culture, one or more markers indicative of the cell culture phenotype, wherein the markers are selected from the group consisting of peptides selected from FIGS. 7 through 138, proteins selected from Tables 2 through 8, and gene expression products from genes selected from Tables 9 through 20 and 24 through 30.

29. (canceled)

30. An engineered cell line with an improved cellular productivity comprising a population of engineered cells, each of which comprising an engineered construct up-regulating or down-regulating one or more genes or proteins selected from Tables 2, 3, 9, 10, 11, and 12.

31. (canceled)

32. (canceled)

33. An engineered cell line with an improved cell growth rate comprising a population of engineered cells, each of which comprising an engineered construct up-regulating or down-regulating one or more genes or proteins selected from Tables 4, 5, 6, 13, 14, 27 and 28.

34. (canceled)

35. (canceled)

36. An engineered cell line with an improved peak cell density comprising a population of engineered cells, each of which comprising an engineered construct up-regulating or down-regulating one or more genes or proteins selected from Tables 8, 15, 16, and 17.

37. (canceled)

38. (canceled)

39. An engineered cell line with an improved sustained cell viability comprising a population of engineered cells, each of which comprising an engineered construct up-regulating or down-regulating one or more genes or proteins selected from Tables 7, 18 and 19.

40. (canceled)

41. (canceled)

42. An engineered cell line with modified lactate production or consumption, the engineered cell line comprising a population of engineered cells, each of which comprising an engineered construct up-regulating or down-regulating one or more genes selected from Tables 29 and 30.

43. (canceled)

44. (canceled)

45. An improved cell line comprising a population of engineered cells, each of which comprising an engineered construct up-regulating or down-regulating one or more genes or proteins selected from Tables 20, 24, 25 and 26.

46. (canceled)

47. (canceled)

48. (canceled)

49. An isolated or recombinant nucleic acid comprising a CHO sequence selected from Tables 9, 13, and 15.

50. An isolated or recombinant protein comprising a CHO sequence selected from Tables 2 and 4.
